SEQUENCE LISTING (201 sequences)

SEQ ID NO: 1 - RNA, 21 nt, Arabidopsis thaliana
  source 1..21: mol_type = other RNA
tcggaccaggcttcattcccc

SEQ ID NO: 2 - RNA, 21 nt, Arabidopsis thaliana
  source 1..21: mol_type = other RNA
tcggaccaggcttcattcccc

SEQ ID NO: 3 - RNA, 21 nt, Arabidopsis thaliana
  source 1..21: mol_type = other RNA
tcggaccaggcttcattcccc

SEQ ID NO: 4 - RNA, 21 nt, Arabidopsis thaliana
  source 1..21: mol_type = other RNA
ttccacagctttcttgaactg

SEQ ID NO: 5 - RNA, 21 nt, Arabidopsis thaliana
  source 1..21: mol_type = other RNA
ttccacagctttcttgaactt

SEQ ID NO: 6 - RNA, 20 nt, Arabidopsis thaliana
  source 1..20: mol_type = other RNA
tgacagaagagagtgagcac

SEQ ID NO: 7 - RNA, 21 nt, Arabidopsis thaliana
  source 1..21: mol_type = other RNA
tcgcttggtgcaggtcgggaa

SEQ ID NO: 8 - RNA, 20 nt, Arabidopsis thaliana
  source 1..20: mol_type = other RNA
tgacagaagagagtgagcac

SEQ ID NO: 9 - RNA, 21 nt, Arabidopsis thaliana
  source 1..21: mol_type = other RNA
tcgataaacctctgcatccag

SEQ ID NO: 10 - RNA, 21 nt, Arabidopsis thaliana
  source 1..21: mol_type = other RNA
tcgataaacctctgcatccag

SEQ ID NO: 11 - RNA, 21 nt, Arabidopsis thaliana
  source 1..21: mol_type = other RNA
gttcaataaagctgtgggaag

SEQ ID NO: 12 - RNA, 21 nt, Arabidopsis thaliana
  source 1..21: mol_type = other RNA
tcgcttggtgcaggtcgggaa

SEQ ID NO: 13 - RNA, 20 nt, Arabidopsis thaliana
  source 1..20: mol_type = other RNA
tgacagaagagagtgagcac

SEQ ID NO: 14 - RNA, 20 nt, Arabidopsis thaliana
  source 1..20: mol_type = other RNA
tgacagaagagagtgagcac

SEQ ID NO: 15 - RNA, 20 nt, Arabidopsis thaliana
  source 1..20: mol_type = other RNA
tgacagaagagagtgagcac

SEQ ID NO: 16 - RNA, 21 nt, Arabidopsis thaliana
  source 1..21: mol_type = other RNA
tggagaagcagggcacgtgcg

SEQ ID NO: 17 - RNA, 21 nt, Arabidopsis thaliana
  source 1..21: mol_type = other RNA
atgcactgcctcttccctggc

SEQ ID NO: 18 - RNA, 21 nt, Arabidopsis thaliana
  source 1..21: mol_type = other RNA
tcggaccaggcttcatccccc

SEQ ID NO: 19 - RNA, 21 nt, Arabidopsis thaliana
  source 1..21: mol_type = other RNA
tgcctggctccctgtatgcca

SEQ ID NO: 20 - RNA, 21 nt, Arabidopsis thaliana
  source 1..21: mol_type = other RNA
ttgacagaagatagagagcac

SEQ ID NO: 21 - RNA, 21 nt, Arabidopsis thaliana
  source 1..21: mol_type = other RNA
ttgacagaagatagagagcac

SEQ ID NO: 22 - RNA, 21 nt, Arabidopsis thaliana
  source 1..21: mol_type = other RNA
tggagaagcagggcacgtgca

SEQ ID NO: 23 - RNA, 21 nt, Arabidopsis thaliana
  source 1..21: mol_type = other RNA
ttcttgtggattccttggaaa

SEQ ID NO: 24 - RNA, 21 nt, Arabidopsis thaliana
  source 1..21: mol_type = other RNA
acagtttgtgttttgttttgt

SEQ ID NO: 25 - RNA, 21 nt, Arabidopsis thaliana
  source 1..21: mol_type = other RNA
tggaagaaggtgagacttgca

SEQ ID NO: 26 - RNA, 24 nt, Arabidopsis thaliana
  source 1..24: mol_type = other RNA
tcctgtgtttcctttgatgcgtgg

SEQ ID NO: 27 - RNA, 21 nt, Arabidopsis thaliana
  source 1..21: mol_type = other RNA
ctttgtctacaattttggaaa

SEQ ID NO: 28 - RNA, 21 nt, Arabidopsis thaliana
  source 1..21: mol_type = other RNA
ctgaagtgtttgggggaactc

SEQ ID NO: 29 - RNA, 22 nt, Arabidopsis thaliana
  source 1..22: mol_type = other RNA
ttcgaggcctattaaacctctg

SEQ ID NO: 30 - RNA, 20 nt, Arabidopsis thaliana
  source 1..20: mol_type = other RNA
agaggtgaccattggagatg

SEQ ID NO: 31 - RNA, 21 nt, Arabidopsis thaliana
  source 1..21: mol_type = other RNA
gctaagagcggttctgatgga

SEQ ID NO: 32 - RNA, 21 nt, Arabidopsis thaliana
  source 1..21: mol_type = other RNA
gctaagagcggttctgatgga

SEQ ID NO: 33 - RNA, 21 nt, Arabidopsis thaliana
  source 1..21: mol_type = other RNA
agaagcaaaatgacgactcgg

SEQ ID NO: 34 - RNA, 21 nt, Arabidopsis thaliana
  source 1..21: mol_type = other RNA
acagtttgtgttttgttttgt

SEQ ID NO: 35 - RNA, 21 nt, Arabidopsis thaliana
  source 1..21: mol_type = other RNA
ggaatcttgatgatgctgcat

SEQ ID NO: 36 - RNA, 21 nt, Arabidopsis thaliana
  source 1..21: mol_type = other RNA
ccgtatcttggccttgtcatt

SEQ ID NO: 37 - RNA, 22 nt, Arabidopsis thaliana
  source 1..22: mol_type = other RNA
ttggggacgagatgttttgttg

SEQ ID NO: 38 - RNA, 21 nt, Arabidopsis thaliana
  source 1..21: mol_type = other RNA
tcatcttcatcatcatcgtca

SEQ ID NO: 39 - RNA, 21 nt, Arabidopsis thaliana
  source 1..21: mol_type = other RNA
gatcatgttcgcagtttcacc

SEQ ID NO: 40 - RNA, 20 nt, Arabidopsis thaliana
  source 1..20: mol_type = other RNA
gcagcaccattaagattcac

SEQ ID NO: 41 - RNA, 21 nt, Arabidopsis thaliana
  source 1..21: mol_type = other RNA
cgtagttgcagagcttgacgg

SEQ ID NO: 42 - RNA, 21 nt, Arabidopsis thaliana
  source 1..21: mol_type = other RNA
ccttctcatcgatggtctaga

SEQ ID NO: 43 - RNA, 20 nt, Arabidopsis thaliana
  source 1..20: mol_type = other RNA
gcagcaccattaagattcac

SEQ ID NO: 44 - RNA, 24 nt, Arabidopsis thaliana
  source 1..24: mol_type = other RNA
attaacgctggcggttgcggcagc

SEQ ID NO: 45 - RNA, 22 nt, Arabidopsis thaliana
  source 1..22: mol_type = other RNA
ttggggacgagatgttttgttg

SEQ ID NO: 46 - RNA, 21 nt, Arabidopsis thaliana
  source 1..21: mol_type = other RNA
tggttttggacacgtgaaaat

SEQ ID NO: 47 - RNA, 21 nt, Arabidopsis thaliana
  source 1..21: mol_type = other RNA
tgagccaaggatgacttgccg

SEQ ID NO: 48 - RNA, 21 nt, Arabidopsis thaliana
  source 1..21: mol_type = other RNA
cttcttaagtgctgataatgc

SEQ ID NO: 49 - RNA, 21 nt, Arabidopsis thaliana
  source 1..21: mol_type = other RNA
taactattttgagaagaagtg

SEQ ID NO: 50 - RNA, 21 nt, Arabidopsis thaliana
  source 1..21: mol_type = other RNA
tgagccaaggatgacttgccg

SEQ ID NO: 51 - RNA, 22 nt, Arabidopsis thaliana
  source 1..22: mol_type = other RNA
tcttgcttaaatgagtattcca

SEQ ID NO: 52 - RNA, 22 nt, Arabidopsis thaliana
  source 1..22: mol_type = other RNA
ttgtgttgcgtttctgttgatt

SEQ ID NO: 53 - RNA, 21 nt, Arabidopsis thaliana
  source 1..21: mol_type = other RNA
gcgtatgaggagccatgcata

SEQ ID NO: 54 - RNA, 21 nt, Arabidopsis thaliana
  source 1..21: mol_type = other RNA
tgttaaggagtgttaacggtg

SEQ ID NO: 55 - RNA, 21 nt, Arabidopsis thaliana
  source 1..21: mol_type = other RNA
tgccaaaggagagttgccctg

SEQ ID NO: 56 - RNA, 21 nt, Arabidopsis thaliana
  source 1..21: mol_type = other RNA
tgttaaggagtgttaacggtg

SEQ ID NO: 57 - RNA, 21 nt, Arabidopsis thaliana
  source 1..21: mol_type = other RNA
tgtgttctcaggtcacccctt

SEQ ID NO: 58 - RNA, 21 nt, Arabidopsis thaliana
  source 1..21: mol_type = other RNA
ttcgcaggagagatagcgcca

SEQ ID NO: 59 - RNA, 21 nt, Arabidopsis thaliana
  source 1..21: mol_type = other RNA
ttagagttttctggatactta

SEQ ID NO: 60 - RNA, 21 nt, Arabidopsis thaliana
  source 1..21: mol_type = other RNA
gctctctatacttctgtcacc

SEQ ID NO: 61 - RNA, 21 nt, Arabidopsis thaliana
  source 1..21: mol_type = other RNA
tgccaaaggagagttgccctg

SEQ ID NO: 62 - RNA, 21 nt, Arabidopsis thaliana
  source 1..21: mol_type = other RNA
atttgtacacctagatctgta

SEQ ID NO: 63 - RNA, 21 nt, Arabidopsis thaliana
  source 1..21: mol_type = other RNA
tgttaaggagtgttaacggtg

SEQ ID NO: 64 - RNA, 21 nt, Arabidopsis thaliana
  source 1..21: mol_type = other RNA
tgattggaaatttcgttgact

SEQ ID NO: 65 - RNA, 21 nt, Arabidopsis thaliana
  source 1..21: mol_type = other RNA
aagctcaggagggatagcgcc

SEQ ID NO: 66 - RNA, 22 nt, Arabidopsis thaliana
  source 1..22: mol_type = other RNA
tagaccgatgtcaacaaacaag

SEQ ID NO: 67 - RNA, 21 nt, Arabidopsis thaliana
  source 1..21: mol_type = other RNA
taactaaacattggtgtagta

SEQ ID NO: 68 - RNA, 21 nt, Arabidopsis thaliana
  source 1..21: mol_type = other RNA
tgttaaggagtgttaacggtg

SEQ ID NO: 69 - RNA, 21 nt, Arabidopsis thaliana
  source 1..21: mol_type = other RNA
caatttctagtgggtcgtatt

SEQ ID NO: 70 - RNA, 21 nt, Arabidopsis thaliana
  source 1..21: mol_type = other RNA
aagctcaggagggatagcgcc

SEQ ID NO: 71 - RNA, 22 nt, Arabidopsis thaliana
  source 1..22: mol_type = other RNA
ttggggacgacatcttttgttg

SEQ ID NO: 72 - RNA, 21 nt, Arabidopsis thaliana
  source 1..21: mol_type = other RNA
tggagaagatacgcaagaaag

SEQ ID NO: 73 - RNA, 21 nt, Arabidopsis thaliana
  source 1..21: mol_type = other RNA
acagtggtcatctggtgggct

SEQ ID NO: 74 - RNA, 21 nt, Arabidopsis thaliana
  source 1..21: mol_type = other RNA
ctttatatccgcatttgcgca

SEQ ID NO: 75 - RNA, 24 nt, Arabidopsis thaliana
  source 1..24: mol_type = other RNA
tgggttgagttgagttgagttggc

SEQ ID NO: 76 - RNA, 21 nt, Arabidopsis thaliana
  source 1..21: mol_type = other RNA
ggactgttgtctggctcgagg

SEQ ID NO: 77 - RNA, 21 nt, Arabidopsis thaliana
  source 1..21: mol_type = other RNA
gagctccttgaagttcaatgg

SEQ ID NO: 78 - RNA, 21 nt, Arabidopsis thaliana
  source 1..21: mol_type = other RNA
ggactgttgtctggctcgagg

SEQ ID NO: 79 - RNA, 21 nt, Arabidopsis thaliana
  source 1..21: mol_type = other RNA
tttaggtcgagcttcattgga

SEQ ID NO: 80 - RNA, 21 nt, Arabidopsis thaliana
  source 1..21: mol_type = other RNA
ttggtgttatgtgtagtcttc

SEQ ID NO: 81 - RNA, 21 nt, Arabidopsis thaliana
  source 1..21: mol_type = other RNA
ttagagttttctggatactta

SEQ ID NO: 82 - RNA, 21 nt, Arabidopsis thaliana
  source 1..21: mol_type = other RNA
acaaagttttatactgacaat

SEQ ID NO: 83 - RNA, 21 nt, Arabidopsis thaliana
  source 1..21: mol_type = other RNA
cagccaaggatgacttgccgg

SEQ ID NO: 84 - RNA, 21 nt, Arabidopsis thaliana
  source 1..21: mol_type = other RNA
tttgtgacatctaggtgcttt

SEQ ID NO: 85 - RNA, 21 nt, Arabidopsis thaliana
  source 1..21: mol_type = other RNA
tcaggtatgattgacttcaaa

SEQ ID NO: 86 - RNA, 21 nt, Arabidopsis thaliana
  source 1..21: mol_type = other RNA
tcaaggaacggattttgttaa

SEQ ID NO: 87 - RNA, 21 nt, Arabidopsis thaliana
  source 1..21: mol_type = other RNA
acatatgatctgcatctttgc

SEQ ID NO: 88 - RNA, 21 nt, Arabidopsis thaliana
  source 1..21: mol_type = other RNA
ttagattcacgcacaaactcg

SEQ ID NO: 89 - RNA, 21 nt, Arabidopsis thaliana
  source 1..21: mol_type = other RNA
cacgtgttctactactccaac

SEQ ID NO: 90 - RNA, 21 nt, Arabidopsis thaliana
  source 1..21: mol_type = other RNA
ttcttgcatatgttctttatc

SEQ ID NO: 91 - RNA, 21 nt, Arabidopsis thaliana
  source 1..21: mol_type = other RNA
tcggaccaggcttcatccccc

SEQ ID NO: 92 - RNA, 20 nt, Arabidopsis thaliana
  source 1..20: mol_type = other RNA
acttggctgattctattatt

SEQ ID NO: 93 - RNA, 21 nt, Arabidopsis thaliana
  source 1..21: mol_type = other RNA
ggccggtggtcgcgagaggga

SEQ ID NO: 94 - RNA, 21 nt, Arabidopsis thaliana
  source 1..21: mol_type = other RNA
tggcaggaaagacataatttt

SEQ ID NO: 95 - RNA, 21 nt, Arabidopsis thaliana
  source 1..21: mol_type = other RNA
tgccaaaggagatttgccctg

SEQ ID NO: 96 - RNA, 21 nt, Arabidopsis thaliana
  source 1..21: mol_type = other RNA
tcactggtaccaatcattcca

SEQ ID NO: 97 - RNA, 21 nt, Arabidopsis thaliana
  source 1..21: mol_type = other RNA
atgggacatcgagcatttaat

SEQ ID NO: 98 - RNA, 21 nt, Arabidopsis thaliana
  source 1..21: mol_type = other RNA
tggcttggtttatgtacaccg

SEQ ID NO: 99 - RNA, 21 nt, Arabidopsis thaliana
  source 1..21: mol_type = other RNA
tgggtggcaaacaaagacgac

SEQ ID NO: 100 - RNA, 21 nt, Arabidopsis thaliana
  source 1..21: mol_type = other RNA
tgagaatgcaaatccttagct

SEQ ID NO: 101 - RNA, 21 nt, Arabidopsis thaliana
  source 1..21: mol_type = other RNA
ttgattcccaatccaagcaag

SEQ ID NO: 102 - RNA, 20 nt, Arabidopsis thaliana
  source 1..20: mol_type = other RNA
gttcgaggcacgttgggagg

SEQ ID NO: 103 - RNA, 22 nt, Arabidopsis thaliana
  source 1..22: mol_type = other RNA
taatcctaccaataacttcagc

SEQ ID NO: 104 - RNA, 21 nt, Arabidopsis thaliana
  source 1..21: mol_type = other RNA
atcagtttcttgttcgtttca

SEQ ID NO: 105 - RNA, 21 nt, Arabidopsis thaliana
  source 1..21: mol_type = other RNA
ttgaattgaagtgcttgaatt

SEQ ID NO: 106 - RNA, 21 nt, Arabidopsis thaliana
  source 1..21: mol_type = other RNA
ttagatgaccatcaacaaact

SEQ ID NO: 107 - RNA, 21 nt, Arabidopsis thaliana
  source 1..21: mol_type = other RNA
tatgatcatcagaaaacagtg

SEQ ID NO: 108 - RNA, 21 nt, Arabidopsis thaliana
  source 1..21: mol_type = other RNA
atagtttctcttgttctgcac

SEQ ID NO: 109 - RNA, 21 nt, Arabidopsis thaliana
  source 1..21: mol_type = other RNA
ttttcttctacttcttgcaca

SEQ ID NO: 110 - RNA, 20 nt, Arabidopsis thaliana
  source 1..20: mol_type = other RNA
ataaatcccaacatcttcca

SEQ ID NO: 111 - RNA, 21 nt, Arabidopsis thaliana
  source 1..21: mol_type = other RNA
gcaacatcttcaagattcaga

SEQ ID NO: 112 - RNA, 20 nt, Arabidopsis thaliana
  source 1..20: mol_type = other RNA
tctcgcgcttgtacggcttt

SEQ ID NO: 113 - RNA, 21 nt, Arabidopsis thaliana
  source 1..21: mol_type = other RNA
taaactaatcacggaaatgca

SEQ ID NO: 114 - RNA, 22 nt, Arabidopsis thaliana
  source 1..22: mol_type = other RNA
tgatctcttcgtactcttcttg

SEQ ID NO: 115 - RNA, 23 nt, Arabidopsis thaliana
  source 1..23: mol_type = other RNA
gctcactctctttttgtcataac

SEQ ID NO: 116 - RNA, 24 nt, Arabidopsis thaliana
  source 1..24: mol_type = other RNA
ttaaagctccaccatgagtccaat

SEQ ID NO: 117 - RNA, 21 nt, Arabidopsis thaliana
  source 1..21: mol_type = other RNA
aggtgctgagtgtgctagtgc

SEQ ID NO: 118 - RNA, 20 nt, Arabidopsis thaliana
  source 1..20: mol_type = other RNA
acaaaatccgtctttgaaga

SEQ ID NO: 119 - RNA, 21 nt, Arabidopsis thaliana
  source 1..21: mol_type = other RNA
ttgcttaaagattttctatgt

SEQ ID NO: 120 - RNA, 21 nt, Arabidopsis thaliana
  source 1..21: mol_type = other RNA
ctgaagtgtttggggggactc

SEQ ID NO: 121 - RNA, 21 nt, Arabidopsis thaliana
  source 1..21: mol_type = other RNA
ctgaagtgtttggggggactc

SEQ ID NO: 122 - RNA, 21 nt, Arabidopsis thaliana
  source 1..21: mol_type = other RNA
ttcttcgtgaatatctggcat

SEQ ID NO: 123 - RNA, 21 nt, Arabidopsis thaliana
  source 1..21: mol_type = other RNA
taagctgccagcatgatcttg

SEQ ID NO: 124 - RNA, 21 nt, Arabidopsis thaliana
  source 1..21: mol_type = other RNA
atcatgctatctctttggatt

SEQ ID NO: 125 - RNA, 21 nt, Arabidopsis thaliana
  source 1..21: mol_type = other RNA
ctgaagtgtttggggggactc

SEQ ID NO: 126 - RNA, 22 nt, Homo sapiens
  source 1..22: mol_type = other RNA
aacccgtagatccgaacttgtg

SEQ ID NO: 127 - RNA, 23 nt, Homo sapiens
  source 1..23: mol_type = other RNA
taccctgtagaaccgaatttgtg

SEQ ID NO: 128 - RNA, 21 nt, Homo sapiens
  source 1..21: mol_type = other RNA
tgagatgaagcactgtagctc

SEQ ID NO: 129 - RNA, 23 nt, Homo sapiens
  source 1..23: mol_type = other RNA
taccctgtagatccgaatttgtg

SEQ ID NO: 130 - RNA, 22 nt, Homo sapiens
  source 1..22: mol_type = other RNA
aagctgccagttgaagaactgt

SEQ ID NO: 131 - RNA, 22 nt, Homo sapiens
  source 1..22: mol_type = other RNA
tccctgagaccctaacttgtga

SEQ ID NO: 132 - RNA, 22 nt, Homo sapiens
  source 1..22: mol_type = other RNA
tagcttatcagactgatgttga

SEQ ID NO: 133 - RNA, 22 nt, Homo sapiens
  source 1..22: mol_type = other RNA
cacccgtagaaccgaccttgcg

SEQ ID NO: 134 - RNA, 24 nt, Homo sapiens
  source 1..24: mol_type = other RNA
tccctgagaccctttaacctgtga

SEQ ID NO: 135 - RNA, 22 nt, Homo sapiens
  source 1..22: mol_type = other RNA
tgaggtagtagtttgtgctgtt

SEQ ID NO: 136 - RNA, 23 nt, Homo sapiens
  source 1..23: mol_type = other RNA
tgagaactgaattccataggctg

SEQ ID NO: 137 - RNA, 22 nt, Homo sapiens
  source 1..22: mol_type = other RNA
aacccgtagatccgatcttgtg

SEQ ID NO: 138 - RNA, 22 nt, Homo sapiens
  source 1..22: mol_type = other RNA
tgaggtagtagattgtatagtt

SEQ ID NO: 139 - RNA, 22 nt, Homo sapiens
  source 1..22: mol_type = other RNA
tggctcagttcagcaggaacag

SEQ ID NO: 140 - RNA, 22 nt, Homo sapiens
  source 1..22: mol_type = other RNA
tgaggtagtaggttgtatagtt

SEQ ID NO: 141 - RNA, 22 nt, Homo sapiens
  source 1..22: mol_type = other RNA
ttcaagtaatccaggataggct

SEQ ID NO: 142 - RNA, 22 nt, Homo sapiens
  source 1..22: mol_type = other RNA
tcagtgcactacagaactttgt

SEQ ID NO: 143 - RNA, 19 nt, Homo sapiens
  source 1..19: mol_type = other RNA
aatggatttttggagcagg

SEQ ID NO: 144 - RNA, 22 nt, Homo sapiens
  source 1..22: mol_type = other RNA
acagtagtctgcacattggtta

SEQ ID NO: 145 - RNA, 22 nt, Homo sapiens
  source 1..22: mol_type = other RNA
acagtagtctgcacattggtta

SEQ ID NO: 146 - RNA, 23 nt, Homo sapiens
  source 1..23: mol_type = other RNA
cccagtgttcagactacctgttc

SEQ ID NO: 147 - RNA, 21 nt, Homo sapiens
  source 1..21: mol_type = other RNA
atcacattgccagggatttcc

SEQ ID NO: 148 - RNA, 22 nt, Homo sapiens
  source 1..22: mol_type = other RNA
tattgcacttgtcccggcctgt

SEQ ID NO: 149 - RNA, 22 nt, Homo sapiens
  source 1..22: mol_type = other RNA
tcggatccgtctgagcttggct

SEQ ID NO: 150 - RNA, 21 nt, Homo sapiens
  source 1..21: mol_type = other RNA
tcagtgcatgacagaacttgg

SEQ ID NO: 151 - RNA, 22 nt, Homo sapiens
  source 1..22: mol_type = other RNA
tgagaactgaattccatgggtt

SEQ ID NO: 152 - RNA, 22 nt, Homo sapiens
  source 1..22: mol_type = other RNA
tgaggtagtagtttgtacagtt

SEQ ID NO: 153 - RNA, 23 nt, Homo sapiens
  source 1..23: mol_type = other RNA
caacggaatcccaaaagcagctg

SEQ ID NO: 154 - RNA, 22 nt, Homo sapiens
  source 1..22: mol_type = other RNA
tagcaccatctgaaatcggtta

SEQ ID NO: 155 - RNA, 21 nt, Homo sapiens
  source 1..21: mol_type = other RNA
ctagactgaagctccttgagg

SEQ ID NO: 156 - RNA, 22 nt, Homo sapiens
  source 1..22: mol_type = other RNA
tgaggtagtaggttgtgtggtt

SEQ ID NO: 157 - RNA, 23 nt, Homo sapiens
  source 1..23: mol_type = other RNA
cccagtgtttagactatctgttc

SEQ ID NO: 158 - RNA, 22 nt, Homo sapiens
  source 1..22: mol_type = other RNA
ggattcctggaaatactgttct

SEQ ID NO: 159 - RNA, 23 nt, Homo sapiens
  source 1..23: mol_type = other RNA
agctacattgtctgctgggtttc

SEQ ID NO: 160 - RNA, 21 nt, Homo sapiens
  source 1..21: mol_type = other RNA
agctacatctggctactgggt

SEQ ID NO: 161 - RNA, 21 nt, Homo sapiens
  source 1..21: mol_type = other RNA
ttcacagtggctaagttctgc

SEQ ID NO: 162 - RNA, 22 nt, Homo sapiens
  source 1..22: mol_type = other RNA
tgtaaacatccccgactggaag

SEQ ID NO: 163 - RNA, 22 nt, Homo sapiens
  source 1..22: mol_type = other RNA
cattgcacttgtctcggtctga

SEQ ID NO: 164 - RNA, 21 nt, Homo sapiens
  source 1..21: mol_type = other RNA
tacagtactgtgataactgaa

SEQ ID NO: 165 - RNA, 22 nt, Homo sapiens
  source 1..22: mol_type = other RNA
acctggcatacaatgtagattt

SEQ ID NO: 166 - RNA, 23 nt, Homo sapiens
  source 1..23: mol_type = other RNA
agctcggtctgaggcccctcagt

SEQ ID NO: 167 - RNA, 22 nt, Homo sapiens
  source 1..22: mol_type = other RNA
caaagaattctccttttgggct

SEQ ID NO: 168 - RNA, 22 nt, Homo sapiens
  source 1..22: mol_type = other RNA
tgtaaacatcctcgactggaag

SEQ ID NO: 169 - RNA, 21 nt, Homo sapiens
  source 1..21: mol_type = other RNA
tcgaggagctcacagtctagt

SEQ ID NO: 170 - RNA, 18 nt, Homo sapiens
  source 1..18: mol_type = other RNA
tcgaggagctcacagtct

SEQ ID NO: 171 - RNA, 21 nt, Homo sapiens
  source 1..21: mol_type = other RNA
ttcacagtggctaagttccgc

SEQ ID NO: 172 - RNA, 25 nt, Homo sapiens
  source 1..25: mol_type = other RNA
tcaagtcactagtggttccgtttag

SEQ ID NO: 173 - RNA, 23 nt, Homo sapiens
  source 1..23: mol_type = other RNA
aacattcaacgctgtcggtgagt

SEQ ID NO: 174 - RNA, 22 nt, Homo sapiens
  source 1..22: mol_type = other RNA
cactagattgtgagctcctgga

SEQ ID NO: 175 - RNA, 22 nt, Homo sapiens
  source 1..22: mol_type = other RNA
gccttctcttcccggttcttcc

SEQ ID NO: 176 - RNA, 22 nt, Homo sapiens
  source 1..22: mol_type = other RNA
actggacttggagtcagaaggc

SEQ ID NO: 177 - RNA, 24 nt, Homo sapiens
  source 1..24: mol_type = other RNA
ttaatgctaatcgtgataggggtt

SEQ ID NO: 178 - RNA, 22 nt, Homo sapiens
  source 1..22: mol_type = other RNA
tgggtctttgcgggcgagatga

SEQ ID NO: 179 - RNA, 22 nt, Homo sapiens
  source 1..22: mol_type = other RNA
tgaggtagtaggttgtatggtt

SEQ ID NO: 180 - RNA, 21 nt, Homo sapiens
  source 1..21: mol_type = other RNA
caacaccagtcgatgggctgt

SEQ ID NO: 181 - RNA, 22 nt, Homo sapiens
  source 1..22: mol_type = other RNA
taggtagtttcatgttgttggg

SEQ ID NO: 182 - RNA, 22 nt, Homo sapiens
  source 1..22: mol_type = other RNA
tcagtgcatcacagaactttgt

SEQ ID NO: 183 - RNA, 23 nt, Homo sapiens
  source 1..23: mol_type = other RNA
aggttacccgagcaactttgcat

SEQ ID NO: 184 - RNA, 22 nt, Homo sapiens
  source 1..22: mol_type = other RNA
tattgcactcgtcccggcctcc

SEQ ID NO: 185 - RNA, 21 nt, Homo sapiens
  source 1..21: mol_type = other RNA
taccacagggtagaaccacgg

SEQ ID NO: 186 - RNA, 22 nt, Homo sapiens
  source 1..22: mol_type = other RNA
tcctgtactgagctgccccgag

SEQ ID NO: 187 - RNA, 22 nt, Homo sapiens
  source 1..22: mol_type = other RNA
tgtaaacatccttgactggaag

SEQ ID NO: 188 - RNA, 22 nt, Homo sapiens
  source 1..22: mol_type = other RNA
tccgagcctgggtctccctctt

SEQ ID NO: 189 - RNA, 22 nt, Homo sapiens
  source 1..22: mol_type = other RNA
gaatgttgctcggtgaacccct

SEQ ID NO: 190 - RNA, 22 nt, Homo sapiens
  source 1..22: mol_type = other RNA
aatgcacccgggcaaggattct

SEQ ID NO: 191 - RNA, 21 nt, Homo sapiens
  source 1..21: mol_type = other RNA
ctgacctatgaattgacagcc

SEQ ID NO: 192 - RNA, 23 nt, Homo sapiens
  source 1..23: mol_type = other RNA
agcagcattgtacagggctatga

SEQ ID NO: 193 - RNA, 21 nt, Homo sapiens
  source 1..21: mol_type = other RNA
ttcaagtaattcaggataggt

SEQ ID NO: 194 - RNA, 22 nt, Homo sapiens
  source 1..22: mol_type = other RNA
cttggcacctagcaagcactca

SEQ ID NO: 195 - RNA, 22 nt, Homo sapiens
  source 1..22: mol_type = other RNA
atgcacctgggcaaggattctg

SEQ ID NO: 196 - RNA, 23 nt, Homo sapiens
  source 1..23: mol_type = other RNA
gtccagttttcccaggaatccct

SEQ ID NO: 197 - RNA, 22 nt, Homo sapiens
  source 1..22: mol_type = other RNA
tcaggctcagtcccctcccgat

SEQ ID NO: 198 - RNA, 22 nt, Homo sapiens
  source 1..22: mol_type = other RNA
tcacaagtcaggctcttgggac

SEQ ID NO: 199 - RNA, 22 nt, Homo sapiens
  source 1..22: mol_type = other RNA
ttatcagaatctccaggggtac

SEQ ID NO: 200 - RNA, 23 nt, Homo sapiens
  source 1..23: mol_type = other RNA
tagcagcgggaacagttctgcag

SEQ ID NO: 201 - RNA, 21 nt, Homo sapiens
  source 1..21: mol_type = other RNA
tggtagactatggaacgtagg

(19) United States
(12) Patent Application Publication
(10) Pub. No.: US 2026/0345505 A1
(43) Pub. Date: Nov. 5, 2026

(54) COMPOSITIONS CONTAINING ISOLATED PLANT-DERIVED AND ADIPOSE STEM CELL-DERIVED EXOSOMES

(71) Applicant: Exotropin LLC, New York, NY (US)

(72) Inventors: Jodi Gurney, Park Ridge, IL (US); Robin Smith, New York, NY (US)

(73) Assignee: Exotropin LLC, New York, NY (US)

(21) Appl. No.: 19/653,782

(22) Filed: Apr. 21, 2026

Related U.S. Application Data
(60) Provisional application No. 63/792,609, filed on Apr. 22, 2025.

(51) Int. Cl. A61K 9/50; A61K 8/14; A61K 8/60; A61K 31/7105; A61P 15/02; A61P 17/00; A61P 17/02; A61Q 5/00; A61Q 19/00

(52) U.S. Cl. CPC A61K 9/5068; A61K 8/14; A61K 8/606; A61K 31/7105; A61P 15/02; A61P 17/00; A61P 17/02; A61Q 5/00; A61Q 19/00

(57) ABSTRACT

The present disclosure provides a composition containing a purified population of extracellular vesicles (EVs) isolated from one or more biological samples, wherein the purified population of EVs comprise a tuned cargo comprising a protein signature and an miRNA signature, wherein the tuned cargo of the purified population of EVs is a result of exposure of the one or more biological samples to one or more stress conditions that cause the one or more biological samples to modulate the EV cargo composition. The tuned cargo of the purified population of EVs can modulate bioactivities of recipient cells. The present disclosure also provides a method for improving appearance of human skin, treating eczema, promoting wound healing, promoting hair health, and promoting vaginal health, comprising applying a composition comprising the purified population of EVs comprising a tuned cargo and a carrier; and applying the composition topically.

### CROSS-REFERENCE TO RELATED APPLICATIONS

This application claims the benefit of priority to U.S. Provisional Application No. 63/792,609 (filed Apr. 22, 2025), which is incorporated by reference herein in its entirety.

### FIELD OF THE INVENTION

The present disclosure relates to compositions comprising extracellular vesicles derived from Aloe vera plants and adipose-derived stem cells, methods of producing such extracellular vesicles, and methods of using such compositions for therapeutic and cosmetic applications.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 20, 2026, is named 2751-00102US-SEQLIST-ST26.xml and is 177,938 bytes in size. The sequence listing contained in this .XML file is part of the specification and is hereby incorporated by reference herein in its entirety.

### BACKGROUND OF THE INVENTION

### Extracellular Vesicles

Extracellular vesicles (EVs) are nano-sized (typically 50-200 nm in diameter) vesicles of different sizes, cargo, and surface markers that are secreted into the extracellular environment through a variety of mechanisms. They carry various components of the cytoplasm and cell membrane that are selectively loaded into these vesicles. They are secreted by all forms of living cells and play essential roles in different physiological functions and pathological processes. They also have been utilized as diagnostic markers and therapeutic tools in several conditions.

Three types of EVs are biologically distinguishable from one another via the distinct processes through which they are released by a cell. However, the experimental classification of these vesicles is less clear, as there is no consensus on what criteria to use for their differentiation. Microvesicles (MVs), or ectosomes (200-2000 nm) arise by direct budding through the cell membrane to the outside of the cell [Shao, H. et al. Chem Rev. (2018) 118 (4): 1917-1950]. Microvesicles are enriched in integrins, selectins, and CD40. Exosomes are produced through the inward invagination of the endosomal membrane pathway [Id., citing Thery, C. et al. Nat. Rev. Immunol. (2002) 2: 569-79; Kowal, J. et al. Curr. Opin. Cell Biol. (2014) 29: 116-125]. The first invagination of the plasma membrane forms a cup-shaped structure that includes cell-surface proteins and soluble proteins associated with the extracellular milieu. This leads to the de novo formation of an early-sorting endosome (ESE) and in some cases may directly merge with a preexisting ESE.

The trans-Golgi network and endoplasmic reticulum can also contribute to the formation and the content of the ESE (Kalluri, R., LeBleu, VS. Science (2020) 367 (6478): eaau6977, citing Kalluri, R. J. Clin. Invest. (2016) 126: 1208-1215; van Neil, G. et al. Nat. Rev. Mol. Cell Biol. (2018) 19: 213-228; McAndrews, KM, Kalluri, R. Mol. Cancer (2019) 18: 52; Mathieu, M. et al. Nat. Cell Biol. (2019) 21: 9-17; Willms, E. et al. Front. Immunol. (2018) 9: 738; Hessvik, NP, Llorente, A. Cell Mol. Life Sci. (2018) 75: 193-208). Small vesicles can be formed by further inward budding of the limiting membrane inside an endosome, leading to the formation of a multivesicular body (MVB), characterized by the presence of intraluminal vesicles [Shao, H. et al. Chem Rev. (2018) 118 (4): 1917-1950, citing Piper, RC, Katzmann, DJ. Annu. Rev. Cell Dev. Biol. (2007) 23: 519-47].

During this process, cytosolic contents, transmembrane and peripheral proteins are incorporated into the invaginating membrane [Id., citing Hurley, JH, Hanson, P I. Nat. Rev. Mol. Cell Biol. (2010) 11: 556-566]. ESEs can mature into late-sorting endosomes (LSEs) and eventually generate multivesicular bodies (MVBs), which are also called multivesicular endosomes. MVBs form by inward invagination of the endosomal limiting membrane (that is, double invagination of the plasma membrane). MVBs may then fuse with the lysosome, leading to the degradation of vesicular contents [Id., citing Vlassov, A V et al. Biochim. Biophys. Acta (2012) 1820: 940-948]. Alternatively, MVBs may fuse with the plasma membrane of the cell, releasing vesicles in an exocytotic fashion to the extracellular space [Id., citing Colombo, M. et al. Annu. Rev. Cell Dev. Biol. (2014) 30: 255-289, Thery, C. F1000 Biol. Rep. (2011) 3: 15]. The released exosomes are small membrane-bound lipid vesicles that have a diameter ranging from 30-200 nm. Because of the double invagination processes, protein topology in exosomes is in the same orientation as in the plasma membrane of cells. Apoptotic bodies (APBs) (800-5000 nm diameter) arise from the cell during the process of apoptosis and emerge either by separation of the membrane blebs or from apoptopodia that arise during the process of apoptosis [Xu X., et al. Biosci. Rep. (2019) 39 (1): BSR20180992].

Collectively, EVs contain an abundance of cellular cargos [Id., citing Kalra, H. et al. PLoS Biol. (2012) 10: e100450; Keerthikumar, S. et al. J. Mol. Biol. (2016) 428: 688-692; Choi, D S et al. Mass Spectrom. Rev. (2015) 34: 474-90]. Consistent with their biogenesis, the membrane composition of microvesicles reflects most closely the plasma membrane of the parent cells [Id., citing Yanez-Mo, M. et al. J. Extracell. Vesicles (2015) 4: 27066]. Consistent with their endosomal origin, the lipid membrane of exosomes is rich in cholesterol, sphingomyelin, and ceramide that are typical of lipid rafts [Tan, S S H et al. Tissue Engineering: Part B (2020) doi: 10.1089/ten.teb.2019.0326].

In contrast, a specific subset of endosomal proteins has been identified in exosomes, suggesting a sorting mechanism during exosomal development. The endosomal sorting complex required for transport (ESCRT) has been extensively characterized for regulating and channeling specific molecules into the intraluminal vesicles of the MVBs [Shao, H. et al. Chem Rev. (2018) 118 (4): 1917-1950, citing Hurley, JH, Hanson, P I. Nat. Rev. Mol. Cell Biol. (2010) 11: 556-66; Henne, W M et al. Dev. Cell (2011) 21: 77-91]. The ESCRT, with its four main complexes (ESCRT 0, I, II, and III) is responsible for delivering ubiquitinated proteins for lysosomal degradation and protein recycling [Id., citing Wollert, T., Hurley, JH. Nature (2010) 464: 864-869]. Studies have shown that the depletion of specific ESCRT-family proteins can alter the protein content of exosomes and the rate of exosome release from cells [Id., citing Colombo, M. et al., J. Cell Sci. (2013) 126: 5553-5565]. Components of the ESCRT system, such as TSG101 and Alix [Id., citing Kowal, J. et al. Curr. Opin. Cell Biol. (2014) 29: 116-125] are found enriched in exosomes and thus are used as markers for exosome identification [Id., citing Lotvall, J. et al. J. Extracell. Vesicles (2014) 3: 26913].

Other ESCRT-independent processes also seem to participate, possibly in an intertwined manner, in exosome formation and release. As such, exosomes are also enriched with molecules involved in ESCRT-independent mechanisms. For example, the tetraspanin proteins such as CD9, CD63 and CD81 have been shown to participate in endosomal vesicle trafficking [Id., citing van Neil, G. et al. Dev. Cell. (2011) 21: 708-721; Verweij, F J et al., EMBO J. (201: 2115 2115-2129]. The involvement of the Rab family of small GTPases in vesicle trafficking and fusion with the plasma membrane also suggests a role of these proteins in exosome release [Id., citing Vanlandingham, PA, Ceresa, BP. J. Biol. Chem. (2009) 284: 12110-12124; Ostrowski, M. et al. Nat. Cell Biol. (2010) 12: 19-30; Zeigerer, A. et al. Nature (2012) 485: 465-470]. In addition, sphingomyelinase has been demonstrated to be involved in vesicle release, as supported by elevated levels of ceramide in exosomes and a reduction in exosome release upon inhibition of sphigomyelinase [Id., citing Trajkovic, K. et al. Science (2008) 319: 1244-1247].

### Mammalian Exosomes: General Principles

Exosomes are released by most if not all mammalian cell types, including platelets, blood cells, dendritic cells, mast cells, T cells, B cells, epithelial cells, endothelial cells, mesenchymal stem cells, smooth muscle cells, neuronal cells and many tumor cells [Zhang, J. et al. Genomics Proteomics Bioinformatics (2015) 13: 17-24, citing Liao J., et al. Int J Mol Sci. 2014; 15:15530-15551; Kopers-Lalic, D. et al. Adv. Drug delivery rev. (2012) doi: 10.1016/j.addr.2012.07.006, citing Thery V., et al. Nat. Rev. Immunol. (2002) 2: 569-579].

Mammalian exosomes, which consist of lipid membranes, are spherical nanovesicles with a diameter of 40-150 nm (approximately 100 nm on average) [Kim, J. et al. Asian J. of Pharmaceutical Sci. (2022) 17: 53-69, citing Sinha, D. et al. Cancers (2021) 13 (2): 326; Villa, F. et al. Pharmaceutics (2019) 11 (11): 557; Zhang, Y. et al. Cell Biosi. (2019) 9 (1): 19; Doyle, L M and Wang, M Z. Cells (2019) 8 (7): 727]. They are constitutively generated by the inward budding of the plasma membrane to form early endosomes. The partial early endosomes integrate the surrounding lamina to generate intraluminal vesicles (ILVs), which encapsulate exosomes within large intracellular multivesicular bodies (MVBs).

The subsequent fusion of MVBs with the plasma membrane leads to the secretion of exosomes from most ILVs into the extracellular space [Id., citing Zhang, Y. et al. Cell Biosci. (2019) 9 (1): 19; Hessvik, N P and Llorente, A. Cell Mol. Life Sci. (2018) 75 (2): 193-208; Farooqi, A A et al. Biotechnol. Adv. (2018) 36 (1): 328-334; Joshi, B S et al. ACS Nano (2020) 14 (4): 4444-4455]. Mammalian exosomes are typically defined by their size, composition and specific exosome marker proteins, such as CD9, CD81, CD63, flotillin and TSG101 [Id., citing Zhang, Y. et al. Cell Biosci. (2019) (1): 19; Yue, B. et al. Cell Prolif (2020) 53 (7): e12857; Kalluri, R. and LeBleu, VS. Science (2020) 367 (6378): eaau6977]. Mammalian exosome architecture, components, and molecular processing reflect the processes taking place in their cells of origin [Id., citing Hu, Q. et al. Precis. Clin. Med. (2020) 3 (1): 54-66; Gluszko, A. et al. Biomed. Res. Int. (2019) 2019: 1628029].

Most mammalian EVs comprising exosomes share a core set of proteins and lipids. Mammalian-derived exosomes are characterized by the presence of specific lipids, such as phosphatidylserine, cholesterol, sphingomyelins, and ceramides. There seems to be a conserved protein repertoire in exosomes across cell-types and species [Kopers-Lalic, D. et al. Adv. Drug delivery rev. (2012) doi: 10.1016/j.addr.2012.07.006, citing Simpson, R J, et al. Proteomics (2008) 8: 4083-4099].

For example, the endosomal proteins such as Alix and TSG101, which are components of the mammalian ESCRT system, have been identified in the majority of the exosomes studied for their protein content thus far. In addition, heat shock proteins, which are involved in protein trafficking, are frequently found in exosomes [Id., citing van Dommelen, S M et al. J. Control. Release (2011) 161: 635-644]. Mammalian exosomes are further enriched in tetraspanins, like CD9, CD63, CD81 and CD82, which are important molecules for protein-protein interactions in cellular membranes. Tetraspanins bind many proteins, including integrins and MHC molecules [Id., citing Thery, C., et al. Nat. Rev. Immunol. (2009) 9: 581-593; Escola, J M. J. Biol. Chem. (1998) 273: 20121-20127; Keller, S. et al. Immunol. Lett (2006) 107: 102-108; Stoorvogel, W. et al. Traffic (2002) 3: 321-330].

Specific Rab proteins, a highly conserved family of small GTPases that function as molecular switches and coordinate membrane traffic [Id., citing Stenmark, H. Nat. Rev. Mol. Cell Biol. (2009) 10: 513-525], are often observed in exosomes by mass-spectrometry. Exosomes are also rich in annexins, membrane trafficking proteins that are involved in fusion events. Furthermore, cytoskeletal proteins like myosin, actin, and tubulin are present in exosomes. Finally, metabolic enzymes, antigen presentation molecules, ribosomal proteins and signal transduction molecules have been shown to be present in exosomes [Id., citing Mathivanan, S., et al. Proteomics (2008) 8: 4083-4099].

Mammalian exosomes may also carry (functional) genetic material, most notably small RNA molecules [Kopers-Lalic, D. et al. Adv. Drug delivery rev. (2012) doi: 10.1016/j.addr.2012.07.006., citing Zomer, A. et al. Commun. Integr. Biol. (2010) 3: 447-450; Gibbings, D, Voinnet, O. Trends Cell Biol. (2010) 20: 491-501]. Of all RNA molecules detected in exosomes, the class of 22 nucleotides (nt) long, non-coding microRNAs (miRNAs) has received attention since the discovery that miRNAs can be functionally transferred to recipient cells [Id., citing Pegtel, D M et al. Proc. Nat. Acad. Sci. USA (2010) 107: 6328-6333, Valadi, H. et al. Nat. Cell Biol. (2007) 9: 654-659].

miRNAs regulate gene expression by binding imperfectly to the 3′ untranslated region of a target mRNA that results in translational repression of the mRNA into protein [Id., citing Bartel, DP. Cell (2004) 116: 281-297; Brennecke, J. et al. PLoS Biol. (20): e85 e85]. The term “non-coding RNAs (ncRNAs”) as used herein refers to functional RNA molecules that are transcribed from DNA but not translated into proteins. High-throughput sequencing technology confirmed that over 98% of the human genome is transcribed into ncRNAs, which are divided into two main groups: the small non-coding RNAs (<200 nucleotides) and the long non-coding RNAs (lncRNAs) (>200 nucleotides). In general, ncRNAs play a role in heterochromatin formation, histone modification and DNA methylation, leading them to regulate gene expression at the transcriptional and post-transcriptional level. Epigenetic related ncRNAs include miRNA, siRNA, piRNA and lncRNA. Non-coding RNA types are summarized in Table 1.

### Extracellular Vesicle Binding

EV binding is mediated by receptors that interact with either universal EV molecules, such as lipids and carbohydrates, or specific peptides present on subsets of EVs. Following initial binding, cells internalize EVs by processes that include receptor-mediated phagocytosis or endocytosis via receptors that include T cell immunoglobulin- and mucin-domain-containing molecule-4 (TIM4), which binds to phosphatidylserine (PS) on EVs; scavenger receptors; integrins; and complement receptors (Gyorgy B, et al. Annu Rev Pharmacol Toxicol. (2015) 55: 439-464, citing Record M, et al. Biochem. Pharmacol. 2011; 81: 1171-1182).

How EV cargo is released into the cytoplasm after entry into recipient cells is unclear. Furthermore, uptake of cargo into a cell is not equivalent to cargo functionality. For instance, EVs may potentially pass through cells within the multivesicular body compartment, which could explain how EVs cross the blood-brain barrier (BBB) (i.e., via a transendothelial route). Endocytotic mechanisms must circumvent the lysosomal degradative pathway, and direct fusion between the EV and target cell plasma membrane or endocytotic membrane does not always ensure functionality of the contents. In many cases, EV cargo can be degraded by recipient cells, thereby inhibiting therapeutic delivery but limiting the impact of off-target delivery. In general, the fate of EVs within the body and cells remains poorly understood and requires additional investigation to elucidate how these processes impact functional EV-mediated delivery (Id.).

EVs comprising exosomes are released by most if not all cell types, including platelets, blood cells, dendritic cells, mast cells, T cells, B cells, epithelial cells, endothelial cells, mesenchymal stem cells, smooth muscle cells, neuronal cells and many tumor cells [Zhang, J. et al. Genomics Proteomics Bioinformatics (2015) 13: 17-24, citing Liao J., et al. Int J Mol Sci. 2014; 15:15530-15551; Kopers-Lalic, D. et al. Adv. Drug delivery rev. (2012) doi: 10.1016/j.addr.2012.07.006, citing Thery V., et al. Nat. Rev. Immunol. (2002) 2: 569-79].

Most EVs comprising exosomes share a core set of proteins and lipids; there seems to be a clear conserved protein repertoire in exosomes across cell-types and species [Kopers-Lalic, D. et al. Adv. Drug delivery rev. (2012) doi: 10.1016/j.addr.2012.07.006, citing Simpson, R J, et al. Proteomics (2008) 8: 4083-99]. For example, the endosomal proteins such as Alix and TSG101 have been identified in the majority of the exosomes studied for their protein content thus far. In addition, heat shock proteins, which are involved in protein trafficking, are frequently found in exosomes [Id., citing van Dommelen, S M et al. J. Control. Release (2011) 161: 635-44]. Exosomes are further enriched in tetraspanins, like CD9, CD63, CD81 and CD82, which are important molecules for protein-protein interactions in cellular membranes.

Tetraspanins bind many proteins, including integrins and MHC molecules [Id., citing Thery, C., et al. Nat. Rev. Immunol. (2009) 9: 581-93; Escola, J M. J. Biol. Chem. (1998) 273: 20121-20127; Keller, S. et al. Immunol. Lett (2006) 107: 102-108; Stoorvogel, W. et al. Traffic (2002) 3: 321-330]. Specific Rab proteins, a highly conserved family of small GTPases that functional as molecular switches and coordinate membrane traffic [Id., citing Stenmark, H. Nat. Rev. Mol. Cell Biol. (2009) 10: 513-525], are often observed in exosomes by mass-spectrometry. Exosomes are also rich in annexins, membrane trafficking proteins that are involved in fusion events. Furthermore, cytoskeletal proteins like myosin, actin and tubulin are present in exosomes. Finally, metabolic enzymes, antigen presentation molecules, ribosomal proteins and signal transduction molecules have been shown to be present in exosomes [Id., citing Mathivanan, S., et al. Proteomics (2008) 8: 4083-4099].

Besides selected sets of proteins, EVs comprising exosomes also incorporate (functional) nucleic acids, most notably small RNA molecules [Id., citing Zomer, A. et al. Commun. Integr. Biol. (2010) 3: 447-50; Gibbings, D, Voinnet, O. Trends Cell Biol. (2010) 20: 491-501]. Of all RNA molecules detected in exosomes, the class of 22 nt long, non-coding miRNAs has received attention since the discovery that miRNAs can be functionally transferred to recipient cells [Id., citing Pegtel, D M et al. Proc. Nat. Acad. Sci. USA (2010) 107: 6328-6333, Valadi, H. et al. Nat. Cell Biol. (2007) 9: 654-659]. MiRNAs regulate gene expression by binding imperfectly to the 3′ untranslated region of the target mRNA that results in translational repression of the mRNA into protein [Id., citing Bartel, DP. Cell (2004) 116: 281-297; Brennecke, J. et al. PLoS Biol. (2005) 3: e85].

EVs comprising exosomes also contain specific proteins depending on the cell of origin. As examples, exosomes from tumor cells contain tumor antigens, platelet-derived exosomes contain coagulation factors, and exosomes from dendritic cells express toll-like receptor ligands [Zhu, L. et al., Artificial Cells, nanomedicine and biotechnology (2018) 46 (53): S166-S179, citing Jong, A Y et al. J. Extracell. Vesicles (2017) 6: 1294368; Sobo-Vujanovic, A. et al., Cell Immunol. (2014) 289: 119-127]. Exosome-mediated functions vary depending on the condition or the origin of the cells [Id., citing Kim, O Y, et al. Semin. Cell Dev. Biol. (2017) 67: 74-82].

### Trafficking of Exosomes

There are three mechanisms of interaction between EVs comprising exosomes and their recipient cells. First, the transmembrane proteins of exosomes directly interact with the signaling receptors of target cells [Zhang, J. et al. Genomics Proteomics Bioinformatics (2015) 13: 17-24, citing Munich S., Sobo-Vujanovic A., Buchser W. J., Beer-Stolz D., Vujanovic N. L. Dendritic cell exosomes directly kill tumor cells and activate natural killer cells via TNF superfamily ligands. Oncoimmunology. (2012) 1:1074-1083]. Second, the exosomes fuse with the plasma membrane of recipient cells and deliver their content into the cytosol [Id., citing Mulcahy L. A., et al. J Extracell Vesicles. (2014) 3: 10.3402/jev/v3/24641].

Third, the exosomes are internalized into the recipient cells and have two fates. In one case, some engulfed EVs comprising exosomes may merge into endosomes and undergo transcytosis, which will move EVs comprising exosomes across the recipient cells and release them into neighboring cells. In the other case, endosomes fused from engulfed exosomes will mature into lysosomes and undergo degradation [Id., citing Mulcahy L. A., et al. J Extracell Vesicles. (2014) 3: 10.3402/jev/v3/24641, Tian T., et al. J Cell Physiol. 2013; 228:1487-1495]. Some factors influencing internalization of exosomes in recipient cells have been reported.

For example, Koumangoye et al. observed that disruption of exosomal lipid rafts resulted in the inhibition of internalization of exosomes and that annexins, which are related to cell adhesion and growth, were essential for the uptake of exosomes in the breast carcinoma cell line BT-549 [Id., citing Koumangoye R. B., et al. PLoS One. (2011) 6:e24234]. Escrevente et al. described a decrease in exosome uptake after the ovarian carcinoma cell line SKOV3 and its derived exosomes were treated with protease K, which indicated that the proteins mediating exosome internalization are presented on the surface of both the cells and the exosomes [Escrevente C., et al. BMC Cancer. (2011) 11:108].

### Exosome Function

EVs comprising exosomes exert their functions through a number of different mechanisms. They can transfer their cargo or membrane constituents from one cell to another, thus transferring functions between cells [Mathieu, M. et al. Nat. Cell Biol. (2019) 21 (10): 9-17]. They carry molecules on their surface, which can act as ligands that stimulate surface receptors in other cells activating intracellular signaling [Maia, J. et al. Front. Cell Dev. Biol. (2018) 6: 18]. These surface molecules can directly perform functions in the extracellular milieu; for example, exosomes may carry remodeling enzymes on their surface, such as matrix metalloproteinases, heparanases, and hyaluronidases [Nawaz, M. et al. Cells (2018) 7 (10) PMC6210724]. Surface proteins on the EV membrane also may capture external molecules or pathogens to neutralize their effects.

It is believed that exosomes can regulate the bioactivities of recipient cells by the transportation of lipids, proteins, and nucleic acids while circulating in the extracellular space [Zhang, J. et al. Genomics Proteomics Bioinformatics (2015) 13: 17-24, citing Liao J., et al. Int J Mol Sci. (2014)15:15530-15551].

### Extracellular Vesicles Play a Role in Several Normal and Disease Physiological Processes.

### Blood Hemostasis

Molecules on the surface of some EVs, such as tissue factor or phosphatidylserine, work as activators of the coagulation cascade. These EVs, which are available in the blood during injury or endothelial damage, are absent from healthy circulating blood. Normal circulating EVs carry plasminogen activators, which induce fibrinolytic activity, preventing thrombus formation. Additionally, exosomes released from platelets under normal conditions inhibit platelet aggregation (Zarra, M. et al. Intl J. Mol. Sci. (2019) 20 (11 PMX6600675).

### Wound Healing

With regard to the role of miRNAs in the proliferation and differentiation of mesenchymal stem cells (MSCs) in the setting of wound healing (Guo, L. et al. Exptl Hematol. (2011) 39: 608-616, citing Silo, S., et al. DNA Cell Biol. (2007) 26: 227-237), using a skin excision model, altered expression in a panel of miRNAs, including upregulated expression of miR-31, -21, -223, -142, -205, -203, -18b, -19a, -130b, -16, -26b, -125b, and let-7f, and down-regulated expression of miR-133a, -181, -30a-3p, -193b, -30a-5p, -204, -200b, -96, -127, -181c, -182 and -130a was demonstrated at the stage of active granulation formation (Id., citing Zou, Z. et al. Expert Opin. Biol. Thera. (2010) 10: 215-230). Further, TGFβ, a key growth factor elevated in the wound site, was found to stimulate upregulation of miR-21 in mesenchymal stem cells (MSCs) as well as in multipotential C3H10T1/2 cells, and to promote proliferation and differentiation of these cells in vitro [Zou, Z. et al., Expert Opin. Biol. Thera. (2010) 10: 215-230]. Consistently, knockdown of miR-21 in the wound bed delayed the healing process. These results suggest that miR-21 regulates gene expression and, subsequently, the behavior of MSCs in wound healing.

Exosomes derived from bone mesenchymal stem cells preconditioned by stimulation with Fe₃O₄ nanoparticles and a static magnetic field (mag-BMSC-Exos) were reported to enhance wound healing through upregulated miR-21-5p [Wu, D. et al. Intl J. Nanomedicine (2020) 15: 7979-7993]. mag-BMSC-Exos were compared to exosomes derived from bone marrow mesenchymal stem cells (BMSC-exo) without preconditioning. Both were isolated by ultracentrifugation. Wound healing in in vitro experiments, including scratch wound assays, transwell assays, and tube formation assays, and an established in vivo wound healing model were compared as were the miRNA expression profiles between BMSC-Exos and mag-BMSC-Exos.

In vitro studies have shown that exosomes derived from human adipose mesenchymal stem cells (ASCs-Exos) could be taken up and internalized by fibroblasts to stimulate cell migration, proliferation and collagen synthesis in a dose-dependent manner, with increased genes expression of N-cadherin, cyclin-1, proliferating cell nuclear antigen (PCNA) and collagen I and III. In vivo, tracing experiments demonstrated that ASC-Exos could be recruited to soft tissue wound area in a mouse skin incision model and that they significantly accelerated cutaneous wound healing [Hu, L. et al. Sci. Rrep. (2016) 6: 32993].

The efficacy of exosomes derived from human umbilical cord blood (UCB-Exos) on wound healing was evaluated by measuring wound closure rates, histological analysis and immunofluorescence examinations. In vitro, quantitative real-time PCR (qRT-PCR) analysis was performed to detect the expression levels of a class of miRNAs that have positive roles in regulating wound healing. The scratch wound assay, transwell assay and cell counting kit-8 analysis were conducted to assess the effects of UCB-Exos on migration and proliferation of human skin fibroblasts and endothelial cells. Tube formation assay was carried out to test the impact of UCB-Exos on angiogenic tube formation ability of endothelial cells. Meanwhile, by using specific RNA inhibitors or siRNAs, the roles of the candidate miRNA and its target genes in UCB-Exos-induced regulation of function of fibroblasts and endothelial cells were assessed.

In vitro, exosomes derived from human umbilical cord blood (UCB-Exos) could promote the proliferation and migration of fibroblasts and enhance the angiogenic activities of endothelial cells. In vivo, the local transplantation of exosomes derived from human umbilical cord blood (UCB-Exos) into full thickness mouse skin wounds resulted in accelerated re-epithelialization, reduced scar widths, and enhanced angiogenesis. It was concluded that accelerated cutaneous wound healing following local transplantation of UCB-Exos occurred through miR-3p-mediated promotion of angiogenesis and fibroblast function [Hu, Y. et al. Theranostics (2018) 8 (10): 169-184].

### Metabolic Functions

EVs play essential metabolic functions by transferring enzymes and metabolites between cells or by performing extracellular metabolic activities. Studies have shown that EVs secreted by liver hepatocytes contain hundreds of enzymes that belong to the different metabolic pathways. When incubated with rat serum, these EVs changed the metabolic profile of the serum [Royo, F. et al. Sci. Rep. (2017) 7: 42798].

### Extracellular Vesicle Composition

The heterogeneity of exosomes and/or extracellular vesicles is thought to be reflective of their size, content, functional impact on recipient cells, and cellular origin. During their secretion, they acquire surface proteins from their cell of origin. They naturally transport mRNA, miRNA, and proteins between cells.

Biofluids can contain large quantities of EVs that shuttle various molecules from parental cells to other cells, including proteins [Id., citing Graner, M W et al. FASEB J. (2009) 23: 1541-1557; Simpson, R J et al. Proteomics (2009) 6: 267-283; Mathivanan, S. et al. Nucleic Acids, Res. (2012) 40: D1241-D1244], mRNA/miRNA [Id., citing Valadi, H. et al. Nat. Cell Biol. (2007) 9: 654-659; Skog, J. et al. Nat. Cell Biol. (2008) 10: 1470-1476] and DNA [Id., citing Balaj, L. et al. Nat. Commun. (2011) 2: 180]. Extracellular vesicles therefore are mediators of near and long-distance intercellular communication in health and disease and affect various aspects of cell biology.

EV composition is determined not only by the cell type but also by the physiological state of the producer cells. The diversity of mechanisms by which EVs are generated and confer effects provides both opportunities and challenges for developing EV-based therapeutics (Gyorgy B, et al. Annu Rev Pharmacol Toxicol. (2015) 55: 439-464). Many methods are used to isolate EVs, and EV contents and properties overlap with those of the cells of origin and other EV types. Formalizing EV nomenclature and defining attributes is a work in progress. The mechanisms of EV uptake and content delivery (or degradation) vary among EV types and recipient cell types. Elucidating and understanding these processes is critical for harnessing EVs as therapeutic delivery vehicles.

Multiple lines of evidence indicate that EVs can transfer biomolecules to modulate recipient cell state in vivo, for example, following bolus injection of purified or concentrated EVs. However, the extent to which such processes naturally shape cellular function and intercellular communication, particularly under homeostatic conditions, remains poorly understood. Moreover, the relative importance of EV-mediated transfer between proximal cells, for example, when diffusional barriers lead to local accumulation of secreted EVs rather than transfer of EVs via the circulation where EV concentrations may be lower is not understood. EV-mediated signaling is dose-dependent (Id., citing Yu S, et al. J. Immunol. 2007; 178: 6867-6875), so the tuning of EV dose may enable the balancing of potential deleterious and therapeutic effects of EV administration. Understanding the role of EV dose is also important for achieving therapeutic efficacy

### Cargos Enriched in EVs

EV proteins derive mainly from cellular plasma membrane and cytosol but not from other intracellular organelles (e.g., Golgi apparatus, endoplasmic reticulum, and nucleus) [Id., citing Simpson, R J et al. Expert Rev. Proteomics (2009) 6: 267-283; Raimondo, F. et al. Proteomics (2011) 11: 709-720; Choi, D S et al. Mass Spectrum Rev. (2015) 34: 474-490]. This protein constitution of EV is indicative of vesicle biogenesis and cargo sorting [Id., citing Kowal, J. et al. Proc. Natl Acad. Sci. USA (2016) 113: E968-E977]

### Membrane Proteins

In mammalian vesicles, both transmembrane and lipid-bound extracellular proteins (e.g., lactadherin) are found associated with microvesicles and exosomes [Id., citing Lotvall, J. et al. J. Extracell Vesicles. (2014) 3: 26913]. Within the group of transmembrane proteins, exosomes are enriched with tetraspanins (e.g., CD9, CD63, CD81), a superfamily of proteins with four transmembrane domains [Id., citing van Niel, G. et al. Dev. Cell (2011) 21: 708-721; Velrweij, F J et al. EMBOJ. (2011) 30: 2115-2129]. Tetraspanins are involved in membrane trafficking and biosynthetic maturation [Id., citing Perez-Hernandez, D. et al. J. Biol. Chem. (2013) 288: 11649-11661; Andreu, Z., Yanez-Mo, M. Front. Immunol (2014) 5: 442] and are highly expressed in exosomes. Tetraspanins, however, are not uniquely expressed in exosomes alone [Id., citing Lotvall, J. et al. J. Extracell. Vesicles (2014) 3: 26913]. Reflecting their derivation from the plasma membrane of cells, EVs are enriched with specific transmembrane protein receptors (e.g., epidermal growth factor receptors/EGFRs) [Id., citing Al-Nedawi, K. et al. Proc. Natl Acad. Sci. USA (2009) 106: 3794-3799] and adhesion proteins (e.g., epithelial cell adhesion molecule/EpCAM [Id., citing Im, H. et al. Nat. Biotechnol. (2014) 32: 490-495; Tauro, B J et al. Mol. Cell Proteomics (2013) 12: 587-598].

### Intravesicular Proteins

EV-associated intravesicular proteins have diverse functions. They include cytosolic proteins that have membrane- or receptor-binding capacity, such as TSG101, ALIX, annexins and Rabs, which are involved in vesicle trafficking. EVs are also enriched with cytoskeletal proteins (e.g., actins, myosins, tubulins), molecular chaperones (e.g., heat-shock proteins/HSPs), metabolic enzymes (e.g., enolases, glyceraldehyde 3-phosphate dehydrogenase/GAPDH) and ribosomal proteins [Id, citing Lotvall, J. et al. J. Extracell. Vesicles (2014) 3: 26913; Choi, D S et al. Mass Spectrom. Rev. (2015) 34: 474-490]. It has been reported that EV protein cargoes can be effectively transported to and received by recipient cells to elicit potent cellular responses in vitro and in vivo [Lai, C P et al. Nat. Communic. (2015) 6: 7029; Mittelbrunn, M., Sanchez-Madrid, F. Nat. Rev. Mol. Cell Biol. (2012) 13: 328-335].

### Nucleic Acids

Both exosomes and microvesicles also contain nucleic acids including miRNAs, mRNAs [Id., citing Valadi, H. et al. Nat. Cell Biol. (2007) 9: 654-659; Skog, J. et al. Nat. Cell Biol. (2008) 10: 1470-1476], DNA [Id., citing Balaj, L. et al. Nat. Commun. (2011) 2: 180; Thakur, B K et al. Cell Res. (2014) 24: 766-769] and other non-coding RNAs [Id., citing Wei, Z. et al. Nat. Commun. (2017) 8: 1145]. RNA types are summarized in Table 1.

TABLE 1
RNA Types

| RNA | Functions | Coding | Typical Size | |
|---|---|---|---|---|
| mRNA | Protein translation | Yes | 400-12,000 nt, | |
| | | | average ≈2100 nt | |
| microRNA | Post-transcriptional | No | 17-24 | nt |
| (miRNA) | gene silencing | | | |
| Y RNA | Component of Ro60 | No | ≈100 | nt |
| | ribonucleoprotein | | | |
| | particle; initiation | | | |
| | factor for DNA | | | |
| | replication | | | |
| Signal Recognition | Component of SRP | No | ≈280 | nt |
| particle RNA (SRP | ribonucleoprotein | | | |
| RNA) | complex that directs | | | |
| | protein trafficking | | | |
| Transfer RNA | Adapter for matching | No | 76-90 | nt |
| (tRNA) | amino acid to mRNA | | | |
| Ribosomal RNA | RNA component of | No | 185 (1.9 kb) | |
| (rRNA) | ribosomes | | 28S (5.0 kb) | |
| Small nuclear RNA | RNA processing such | No | ≈150 | nt |
| (snRNA) | as mRNA splicing | | | |
| Small nucleolar | Guiding chemical | No | 20-24 | nt |
| RNA (snoRNA) | modifications of other | | | |
| | RNAs | | | |
| Long noncoding | Many, including in- | No | >100 | nt |
| RNA (lncRNA) | transcription and post- | | | |
| | transcription regulation | | | |

mRNA

mRNAs are a large family of coding RNA molecules that specify protein sequence information. Studies have reported that EVs contain a substantial proportion of their parent cells' mRNA pool, many of which are cell type-specific mRNA [Shao, H. et al. Chem Rev. (2018) 118 (4): 1917-1950, citing Wei, Z. et al. Nat. Commun. (2017) 8: 1145; Batagov, AO, Kurochkin, IF. Biol. Direct (2013) 8: 12]. These mRNA molecules, often in fragmented form, reside within EVs and are protected from RNase degradation. Furthermore, the fraction of polyadenylated mRNA molecules in EVs suggest that some of them (<2 kb) are capable of encoding polypeptides in support of protein synthesis (i.e., they have functionality in protein translation). This has been confirmed in multiple studies through different translation assays in recipient cells [Id., citing Valadi, H. et al. Nat. Cell Biol. (2007) 9: 654-659; Skog, J. et al. Nat. Cell Biol. (2008) 10: 1470-1476; Lai, C P et al. Nat. Commun. (2015) 6: 7029].

miRNA

miRNAs are a class of small, noncoding RNAs (typically 17-24 nucleotides), which mediate post-transcriptional gene silencing usually by targeting the 3′ untranslated region of mRNAs. By suppressing protein translation, EV miRNAs are powerful regulators for a wide range of biological processes [Id., citing Mittelbrunn, M. et al. Nat. Commun. (2011) 2: 282; Redzic, J S et al. Semin. Cancer Biol. (2014) 28: 14-23]. miRNAs can also exist in multiple stable forms when circulating in bodily fluids. For example, in addition to being packaged into EVs, circulating miRNAs can also be loaded onto high-density lipoprotein [Id., citing Vickers, K C et al. Nat. Cell Biol. (2011) 13: 423-433; Wagner, J. et al. Arterioscler. Thromb. Vasc. Biol. (2013) 33: 1392-1400] or bound to AGO2 protein outside the vesicles [Id., citing Arroyo, et al. Proc. Natl Acad. Sci. USA (2011) 108: 5003-5008; Turchinovich, A. et al. Methods Mol. Biol. (2013) 1024: 97-107].

The distribution of miRNAs within EVs remains unclear [Id., citing Min, PK & Chan, SY. Eur. J. Clin. Invest. (2015) 45: 860-874; Turchinovich, A. et al. Methods Mol. Biol. (2013) 1024: 97-107; Chevillet, J R et al. Proc. Natl. Acad. Sci. USA (2014) 111: 14888-14893]. As in the case of mRNA, miRNA profiles in EVs reflect their cell of origin but differ somewhat from their parental cells. Some miRNAs have been found preferentially sorted into EVs and remaining functional in recipient cells to regulate protein translation [Id., citing Villarroya-Beltri, C. et al. Nat. Commun. (2013) 4: 2980; Koppers-Lalic, D. et al. Cell Rep. (2014) 8: 1649-1658; Santangelo, L. et al. Cell Rep. (2016) 17: 799-808; Teng, Y. et al. Nat. Commu. (2017) 8: 14448].

### Other RNA Types

In addition to mRNA and miRNA, many noncoding RNA types have been identified in EVs through next generation sequencing [Id., citing Huang, X. et al. BMC Genomics (2013) 14: 319; Conley, A. et al. RNA Biol. (2017) 14: 305-316]. These RNAs include transfer RNA (tRNA), ribosomal RNA (rRNA), small nuclear RNA (snRNA), small nucleolar RNA (snoRNA), as well as long noncoding RNA (lncRNA) [Id., citing Wei, Z. et al. Nat. Commun. (2017) 8: 1145; Huang, X. et al. BMC Genomics (2013) 14: 319; Crescitelli, R. et al. J. Extracell. Vesicles (2013) 2: 20677].

Small (about 20-30 nucleotide (nt)) noncoding RNAs regulate eukaryotic genes and genomes [Carthew, R W and Sontheimer, E J. Cell (2009) 136: 642-655]. This regulation can occur at multiple levels of genome function, including chromatin structure, chromosome segregation, transcription, RNA processing, RNA stability, and translation (Id.). The effects of small RNAs on gene expression and control are generally inhibitory, and the corresponding regulatory mechanisms are therefore collectively subsumed under the heading of RNA silencing (Id.). The central theme that runs throughout is that the small RNAs serve as specificity factors that direct bound effector proteins to target nucleic acid molecules via base-pairing interactions (Id.). Invariably, the core component of the effector machinery is a member of the Argonaute protein superfamily (Id.).

There are three main categories of small RNAs: short interfering RNAs (siRNAs), microRNAs (miRNAs), and piwi-interacting RNAs (piRNAs) (Id.). siRNAs and miRNAs are the most broadly distributed in both phylogenetic and physiological terms and are characterized by the double-stranded nature of their precursors (Id.). In contrast, piRNAs are primarily found in animals, exert their functions most clearly in the germline, and derive from precursors that are poorly understood, but appear to be single stranded (Id.). Where siRNAs and miRNAs bind to members of the Ago clade of Argonaute proteins, piRNAs bind to members of the Piwi clade (Id.).

The signature components of RNA silencing are Dicers, Agos, and ~21-23 nt duplex-derived RNAs (Id.). Both siRNA and miRNA small RNAs depend on Dicer enzymes to excise them from their precursors and Argonaute (Ago) proteins to support their silencing effector functions (Id.).

RNase III enzymes, which are dsRNA-specific nucleases, are the source of miRNA/siRNA biogenesis (Id.). One class of large RNase III enzymes has several domains in a specific order from the amino to carboxy terminus: a DEXD/H ATPase domain, a DUF283 domain, a PAZ domain, two tandem RNase III domains, and a dsRNA-binding domain (Id.). Some members of this family differ slightly from this arrangement (Id.).

The PAZ and RNase III domains play central roles in excising siRNAs preferentially from ends of dsRNA molecules. PAZ domains are shared with Argonaute proteins and are specialized to bind RNA ends, especially duplex ends with short (~2 nt) 3′ overhangs. An end engages the Dicer PAZ domain, and the substrate dsRNA then extends approximately two helical turns along the surface of the protein before it reaches a single processing center that resides in a cleft of an intramolecular dimer involving the RNase III domains.

Each of the two RNase IIII active sites cleaves one of the two strands, leading to staggered duplex scission to generate new ends with ~2-3′ nt overhangs. The reaction leaves a 5′ monophosphate on the product ends, consistent with a requirement for this group during later stages of silencing. This general model pertains equally to pre-miRNA stem-loop substrates and to long, perfectly base-paired dsRNAs. In some species, different functional categories of small RNAs exhibit slightly different lengths; this appears to be dictated by the distance between the PAZ domain and the processing center in the relevant Dicer enzyme (Id.).

The roles of the ATPase domain probably vary among different forms of Dicer (Id.). ATP promotes dsRNA processing by Drosophila Dicer 2 and C. elegans Dcr-1, and mutations predicted to cripple ATPase activity in Drosophila Dicer-2 specifically abolish dsRNA processing. In contrast, ATP is dispensable for dsRNA processing by human Dcr (hDcr), and an ATPase defective mutant exhibits no processing defect (Id.).

Dicers isolated from their natural sources generally are found in a heterodimeric complex with a protein that contains two or three double stranded Ras binding domains (dsRBDs); the Ras-binding domain (RBD) is an independent domain of about 75 residues, which is sufficient for GTP-dependent binding of Ras and other G alpha GTPases. Both hDcr and Drosophila Dcr-2 process dsRNAs effectively in the absence of the heterodimeric partner (TAR RNA binding protein (TRBP) and RNAi defective-2 (R2D2), respectively). In at least some cases, the role of Dicer in silencing extends beyond dsRNA processing and into the pathway of RISC assembly; this activity is much more dependent on the dsRBD partner protein (Id.).

### MicroRNA Biogenesis

MicroRNAs are found in plant and animal branches of Eukaryotes and are encoded by a bewildering array of genes. Transcription of miRNAs is typically performed by RNA polymerase II, and transcripts are capped and polyadenylated. Although some animal miRNAs are individually produced from separate transcription units, many more are produced from transcription units that make more than one product. A transcript may encode clusters of distinct miRNAs, or it may encode miRNA and protein. The latter type of transcript is organized such that the miRNA sequence is located within an intron. Many new animal miRNAs are thought to arise from accumulation of nucleotide sequence changes and not from gene duplication [Carthew, R W and Sontheimer, E J. Cell (2009) 136: 642-655].

The resulting primary or pri-miRNA transcript extends both 5′ and 3′ from the miRNA sequence, and two sequential processing reactions trim the transcript into the mature miRNA. Processing depends on the miRNA sequence folding into a step-loop structure. A typical animal pri-miRNA consists of an imperfectly paired stem of ~33 bp, with a terminal loop and flanking segments. The first processing step, which occurs in the nucleus, excises the stem-loop from the remainder of the transcript to create a pre-miRNA product. For most pri-miRNAs, a nuclear member of the RNase III family (Drosha in animals) carries out this cleavage reaction. Although Drosha catalyzes pri-miRNA processing, it depends on a protein cofactor, which contains two double stranded receptor binding (dsRBD) domains and stably associates with the ribonuclease to form the microprocessor complex (Id.).

An alternative pathway uses splicing of pri-miRNA transcripts to liberate introns that precisely mimic the structural features of pre-miRNAs. These introns then enter the miRNA processing pathway without the aid of the Microprocessor (Id.).

The second processing step excises the terminal loop from the pre-miRNA stem to create a mature miRNA duplex of approximately 22 bp length. In animals, the pre-miRNA is exported from the nucleus, and the canonical Dicer enzyme carries out the cleavage reaction in the cytoplasm (Id.).

MicroRNAs behave like traditional polymeric products of gene activity, such that most species of a miRNA have highly exact ends, although there is a little variation. This feature of miRNAs may allow them to interact with greater specificity on substrate mRNAs without a need for stringent complementarity or large overlap (Id.).

Consequently, the processing machinery is constructed to produce miRNA duplexes with highly exact ends. The first cut, carried out by Drosha with the aid of its dsRBD binding partner protein (called DGCR8), is most critical. DGCR8 directly interacts with the pri-miRNA stem and flanking single-stranded segments. The cleavage site is determined by the distance from the stem-flank junction, which is precisely one turn of a dsRNA helix (11 bp) and is the minimal processing length for an RNase III enzyme. Although Drosha carries out the cleavage reaction, it relies upon DGCR8 to serve as a molecular anchor that properly positions Drosha's catalytic site the correct distance from the stem-flank junction. Thus, the endpoint of the stem is a critical determinant for one end of the mature miRNA (Id.).

The second cut performed by Dicer defines the other end of the mature miRNA. Dicer will cleave anywhere along a dsRNA molecule but has a strong preference for the terminus. The PAZ domain of Dicer interacts with the 3′ overhang at the terminus and determines the cleavage site in a ruler-like fashion. The RNase III catalytic sites are positioned two helical turns or 22 bp away from the terminus/PAZ portion of the Dicer-RNA complex (Id.).

While regulation of miRNA biogenesis has not been extensively studied, a surprising number of miRNA genes are formed under the control of the very targets that they regulate. A rationale behind these double-negative regulatory relationships is that tight regulation of miRNA biogenesis is crucial. Mis-expression of miRNAs frequently mimics loss of function phenotypes for their targets. This would be prevented if biogenesis of a miRNA is strictly controlled by its targets. The restriction would also explain how off-targeting effects by wayward miRNAs are carefully limited (Id.).

### MicroRNA Associations

The mature miRNA duplex is a short-lived entity; it is rapidly unwound when it associates with an Ago protein. Unwinding occurs so rapidly after duplex formation, because the two processes are physically coupled due to Ago2's presence in a complex with Dicer and TRBP, the double-stranded RNA binding protein that loads siRNA into the RISC (Id.).

miRNA unwinding is accompanied by differential strand retention, i.e., one strand is retained while the other strand is lost. Strand retention is based on the relative thermodynamic stability of the duplex's ends. Although the rule is that the 5′ terminus of the retained strand is at the less stably base-paired end of the duplex, this rule is not absolute. The other strand is appreciably detected in Ago complexes, lending ambiguity to the notion of strand asymmetry. Although either strand can become stably associated with Ago proteins, the more commonly associated strand is termed the miRNA strand; the other strand is called the miRNA* strand. miRNA unwinding is not accompanied by cleavage of the ejected strand by the associated Ago (Id.).

The mammalian Dicer/Ag/miRNA complex is associated with other proteins, e.g., Gemin3, Gemin4, Mov10, and Imp8, as well as the mammalian protein GW182, associate with Ago2. GW182 is both necessary and sufficient for miRNA-bound Ago to silence gene expression. Thus miRNA-bound Ago in association with GW182 can be thought of as the miRISC complex (Id.).

Post-Transcriptional Repression by miRNAs

An miRNA acts as an adaptor for miRISC to specifically recognize and regulate particular mRNAs. If miRISC is tethered to a heterologous RNA recognition factor, the factor enables miRISC to recognize and repress mRNAs that lack miRNA-binding sites. With few exceptions, miRNA-binding sites in animal mRNAs lie in the 3′ untranslated region (UTR) and are usually present in multiple copies. Most animal miRNAs bind with mismatches and bulges, although a key feature of recognition involves Watson-Crick base pairing of miRNA nucleotides 2-8, representing the seed region (Id.).

While it was thought that perfect complementarity allows Ago-catalyzed cleavage of the mRNA strand, whereas central mismatches exclude cleavage and promote repression of mRNA translation, it appears that translational repression is the default mechanism by which miRNAs repress gene expression, both in animals and plants. Perfectly complementary miRNAs may additionally engage in mRNA cleavage such that their effects are the result of both mechanisms (Id.).

The mechanisms by which the microRNA-induced silencing complex (miRISC) regulates translation have been subject to ongoing debate. The fundamental issue of whether repression occurs at translation initiation or post-initiation has not yet been resolved. There are three competing models for how miRISC represses initiation. One proposes that there is competition between miRISC and eukaryotic translation initiation factor 4E (elF4E) for binding to the mRNA 5′ cap structure. A second model has proposed that miRISC stimulates de-adenylation of the mRNA tail; translation is repressed because the cap and poly(A)-binding protein 1 (PABP1)-free tail of the deadenylated mRNA are unable to circularize. A third model has proposed that miRISC blocks association of the 60S ribosomal subunit with the 40S preinitiation complex, i.e., the recruitment of eIF6 by miRISC may repress translation by preventing the assembly of translationally competent ribosomes at the start codon (Id.).

It is unclear why some targets are degraded and others are not (Id.).

Without being limited by any particular theory, it appears that the mode of regulation of any miRNA (repression vs. activation) in the context of the whole cell and the myriad activities that affect posttranscriptional gene regulation may be context dependent (Id.).

The cell's position in the cell cycle is one such context. For example, miRNA let-7 and an artificial miRNA (CXCR-4) repress translation in proliferating human cells but change into translational activators when the cell cycle is arrested at the G1 checkpoint by serum starvation. Aphidicollin-(a reversible inhibitor of eukaryotic nuclear DNA replication targeting B-family DNA polymerases) induced arrest at G1 also generates translational activation, whereas nocodazole (a microtubule de-polymerizing agent)-induced arrest at G2/M generates translational repression. Lymphocyte growth arrest induces TNFα expression that is required for macrophage maturation; miR-369-3p switches from a repressor to an activator of TNFα translation when cells in culture are growth arrested (Id., citing Vasudevan, S. et al. Science (2007) 318: 1931-34).

Binding site position is another context. Interaction of miR-10a with the 5′UTR of certain ribosomal subunit mRNAs leads to their activated translation, whereas interaction with the 3′UTR leads to repression (Id., citing Orom, U A et al. (2008) Mol. Cell 30: 460-471).

Another context is how small RNA regulation is organized and modulated within the cell. Ago proteins are frequently associated with membrane trafficking compartments, such as the Golgi and ER (Id., citing Cikaluk, D. E. et al. Mol. Biol. Cell (1999) 10: 3357-3372). It has been hypothesized that miRISC factors might become anchored in certain subcellular compartments, e.g., processing (P) bodies or GW bodies, two separate pools of sequestered non-translating RNAs (Patel, P H, et al. PLos One (2016) 11(3): e015029). Subunits of miRISC (miRNAs, Ago and GW1821) and their repressed targets also are enriched in GW bodies. While GW bodies are not essential for miRNA repression, GW body formation requires an intact miRNA pathway (Carthew, R W and Sontheimer, E J. Cell (2009) 136: 642-655).

miRNA Expression

MicroRNAs regulate gene expression at the post-transcriptional level. The exact functional outcome of an miRNA may be determined by multiple features, including the cell type affected, the inducing signal, and the transcriptomic profile of the cell, which ultimately affect the availability and ability to engage different target mRNAs and bring about the cell's unique responses. Indeed, data suggest that miRNAs may play different roles in diverse biological contexts [Lee, H-M et al. BMB Rep. (2016) 49 (6): 311-318].

### Argonautes

The Argonaute superfamily can be divided into three separate subgroups: the Piwi clade that binds piRNAs, the Ago clade that associates with miRNAs and siRNAs, and a third clade described in nematodes. All gene regulatory phenomena involving ~20-30 nt RNAs are thought to require one or more Argonaute proteins, which are the central, defining components of an RNA-induced silencing complex (RISC). The double-stranded products of Dicer enter into an RISC assembly pathway that involves duplex unwinding, culminating in the stable association of only one of the two strands with the Ago effector protein. This guide strand directs target recognition by Watson-Crick base pairing; the other strand of the original small RNA duplex (the passenger strand) is discarded (Id.).

Argonaute proteins are defined by the presence of four domains: the PAZ domain (shared with Dicer enzymes), the PIWI domain that is unique to the Argonaute superfamily, and the N and Mid domains. The overall protein structure is bi-lobed, with one lobe consisting of the PAZ domain and the other lobe consisting of the PIWI domain flanked by N-terminal (N) and middle (Mid) domains. The Argonaute PAZ domain has RNA 3′ terminus binding activity, and the co-crystal structures reveal that this function is used in guide strand binding. The other end of the guide strand engages a 5′ phosphate binding pocket in the Mid domain, and the remainder of the guide tracks along a positively charged surface to which each of the domains contributes. The protein-DNA contacts are dominated by sugar-phosphate backbone interactions. Guide strand nucleotides 2-6, which are especially important for target recognition, are stacked with their Watson-Crick faces exposed and available for base pairing (Id.).

The PIWI domain adopts an RNase H-like fold that in some cases can catalyze guide strand-dependent endonucleolytic cleavage of a base pair target. This initial cut represents the critical first step in a subset of small RNA silencing events that proceed through RNA destabilization. Not all Argonaute proteins have endonucleolytic activity, and those that lack it usually also lack critical active-site residues that coordinate a presumptive catalytic metal ion (Id.).

In humans, four of the eight Argonaute proteins are from the Ago clade and associate with both siRNAs and miRNAs (Id.).

miRNA Sorting into Exosomes

miRNAs are not randomly incorporated into exosomes. Guduric-Fuchs et al. analyzed miRNA expression levels in a variety of cell lines and their respective derived exosomes and found that a subset of miRNAs (e.g., miR-150, miR-142-3p, and miR-451) preferentially enter exosomes [Zhang, J. et al. Genomics Proteomics Bioinformatics (2015) 13: 17-24, citing Guduric-Fuchs J., et al. BMC Genomics. (2012) 13:357]. Similarly, Ohshima et al. compared the expression levels of let-7 miRNA family members in exosomes derived from the gastric cancer cell line AZ-P7a with those from other cancer cell lines, including the lung cancer cell line SBC-3/DMS-35/NCI-H69, the colorectal cancer cell line SW480/SW620, and the stomach cancer cell line AZ-521. As a result, they found that members of the let-7 miRNA family are abundant in exosomes derived from AZ-P7a but are less abundant in exosomes derived from other cancer cells [Id., citing Ohshima K., et al. PLoS One. 2010; 5: e13247].

Moreover, some reports have shown that exosomal miRNA expression levels are altered under different physiological conditions. The level of miR-21 was lower in exosomes from the serum of healthy donors than those from glioblastoma patients [Id., citing Skog J., et al. Nat Cell Biol. 2008; 10:1470-1476]. Levels of let-7f, miR-20b, and miR-30e-3p were lower in vesicles from the plasma of non-small-cell lung carcinoma patients than normal controls [Id., citing Silva J., et al. Eur Respir J. 2011; 37:617-623]. Different levels of eight exosomal miRNAs, including miR-21 and miR141, were also found between benign tumors and ovarian cancers [Id., citing Taylor D. D., et al. Gynecol Oncol. 2008; 110:13-21].

There are four potential modes for sorting of miRNAs into exosomes, although the underlying mechanisms remain largely unclear. These include: 1) The neural sphingomyelinase 2 (nSMase2)-dependent pathway. nSMase2 is the first molecule reported to be related to miRNA secretion into exosomes. Kosaka et al. found that overexpression of nSMase2 increased the number of exosomal miRNAs, and conversely inhibition of nSMase2 expression reduced the number of exosomal miRNAs [Id., citing Kosaka N., et al. J Biol Chem. (2013) 288:10849-10859].
2) The miRNA motif and sumoylated heterogeneous nuclear ribonucleoproteins (hnRNPs)-dependent pathway. Villarroya-Beltri et al. discovered that sumoylated hnRNPA2B1 could recognize the GGAG motif in the 3′ portion of miRNA sequences and cause specific miRNAs to be packed into exosomes [Id., citing Villarroya-Beltri C., et al. Nat Commun. 2013; 4:2980]. Similarly, another two hnRNP family proteins, hnRNPA1 and hnRNPC, can also bind to exosomal miRNAs, suggesting that they might be candidates for miRNA sorting as well. However, no binding motifs have been identified yet [Id., citing Villarroya-Beltri C., et al. Nat Commun. 2013; 4:2980].
3) The 3′-end of the miRNA sequence-dependent pathway. Koppers-Lalic et al. discovered that the 3′ ends of uridylated endogenous miRNAs were mainly presented in exosomes derived from B cells or urine, whereas the 3′ ends of adenylated endogenous miRNAs were mainly presented in B cells [Id., citing Koppers-Lalic D., et al. Cell Rep. 2014; 8:1649-1658]. The above two selection modes commonly indicate that the 3′ portion or the 3′ end of the miRNA sequence contains a critical sorting signal.
4) The miRNA induced silencing complex (miRISC)-related pathway. It is well known that mature miRNAs can interact with assembly proteins to form a complex called miRISC. The main components of miRISC include miRNA, miRNA-repressible mRNA, GW182, and AGO2. The AGO2 protein in humans, which prefers to bind to U or A at the 5′ end of miRNAs, plays an important role in mediating mRNA:miRNA formation and the consequent translational repression or degradation of the mRNA molecule [Id., citing Frank F., et al. Nature. 2010; 465:818-822]. Recent studies recognized a possible correlation between AGO2 and exosomal miRNA sorting.

The miRNAs in cell-released exosomes can circulate with the associated vehicles to reach neighboring cells and distant cells. After being delivered into acceptor cells, exosomal miRNAs play functional roles. Although it is difficult to completely exclude the effects of other exosomal cargos on recipient cells, miRNAs are considered the key functional elements. The functions of exosomal miRNAs can be generally classified into two types. One is the conventional function, i.e., miRNAs perform negative regulation and confer characteristic changes in the expression levels of target genes. For example, exosomal miR-105 released from the breast cancer cell lines MCF-10A and MDA-MB-231 reduced ZO-1 gene expression in endothelial cells and promoted metastases to the lung and brain [Id., citing Zhou W., et al. Cancer Cell. (2014) 25:501-515].

Exosomal miR-214, derived from the human microvascular endothelial cell line HMEC-1, stimulated migration and angiogenesis in neighboring HMEC-1 cells [Id., citing van Balkom B. W., et al. Blood. (2013) 121: S1-S15]. Exosomal miR-92a, derived from K562 cells, significantly reduced the expression of integrin α5 in the human umbilical vein endothelial (HUVEC) cells and enhanced endothelial cell migration and tube formation [Id., citing Umezu T., et al. Oncogene. (2013) 32:2747-2755]. The other type is a novel function that has been identified in some miRNAs when they are studied as exosomal miRNAs rather than intracellular miRNAs. Exosomal miR-21 and miR-29a, in addition to the classic role of targeting mRNA, were first discovered to have the capacity to act as ligands that bind to toll-like receptors (TLRs) and activate immune cells [Id., citing Fabbri M., et al. Proc Natl Acad Sci USA. (2012) 109: E2110-E2116].

Exosomal miRNAs can stably exist in the blood, urine, and other body fluids of patients, and exosomes can reflect their tissue or cell of origin by the presence of specific surface proteins [Id., citing Simons M., et al. Curr Opin Cell Biol. (2009) 21:575-581, Mathivanan S., et al. J Proteomics. (2010) 73:1907-1920, Gross J. C., et al. Nat Cell Biol. (2012) 14:1036-1045]. Furthermore, the amount and composition of exosomal miRNAs differ between patients with disease and healthy individuals. Thus, exosomal miRNAs show potential for use as noninvasive biomarkers to indicate disease states.

Several previous studies have profiled exosomal miRNAs in different samples. Some exosomal miRNAs can be used to aid in clinical diagnosis [Id., citing Skog J., et al. Nat Cell Biol. (2008)10:1470-1476; Silva J., et al. Eur Respir J. (2011) 37:617-623; Taylor D. D., et al. Gynecol Oncol. (2008) 110:13-21; Rabinowits G., et al. Clin Lung Cancer. (2009) 10:42-46]. For example, a set of exosomal miRNAs, including let-7a, miR-1229, miR-1246, miR-150, miR-21, miR-223, and miR-23a, can be used as the diagnostic biomarker of colorectal cancer [Id., citing Ogata-Kawata H., et al. PLoS One. (2014) 9: e92921]. Another set, miR-1290 and miR-375, can be used as the prognostic marker in castration-resistant prostate cancer [Id., citing Huang X., et al. BMC Genomics. 2013; 14:319].

Besides endogenous miRNAs, exogenous miRNAs can also be sorted into exosome; this has been experimentally confirmed by Pegtel et al. [Id., citing Pegtel D. M., et al. Proc Natl Acad Sci USA. (2010) 107:6328-6333] and Meckes et al. [Id., citing Meckes, DG, Jr. et al. Proc. Natl. Acad. Sci. USA (2010) 107: 20370-75], who observed that human tumor viruses can exploit exosomes as delivery vectors to transfer their exogenous miRNAs to other non-infected cells [Id., citing Pegtel D. M., et al. Proc Natl Acad Sci USA. (2010) 107:6328-6333, Meckes D. G., Jr., et al. Proc Natl Acad Sci USA. (2010) 107:20370-20375]. Hence, exogenous small RNAs have also been transferred by exosomes by mimicking the molecular mechanism of endogenous miRNAs transportation.

### ESCRT in Mammalian Cells

The endosomal sorting complex required for transport (ESCRT) machinery responsible for sorting the cargo proteins of ILVs is divided into four complexes, ESCRT-0, -I, -II, —III, which work cooperatively to generate MVBs with associated proteins VPS4, VTA1, and ALG-2 interacting protein (ALIX).

The ESCRT-0 complex is composed of two subunits, hepatocyte growth factor-regulated tyrosine kinase (HRS) and STAM, both of which can engage ubiquitinated substrates destined for lysosomal degradation. EXCRIT-0 recruits TSG101 of the ESCRT-I complex and isolates the ubiquitinated proteins in the endosomal membrane. The ESCRT-I complex is essential and responsible for cargo sorting in the MVBs, as it induces budding by deforming the plasma membrane.

Subsequently, ESCRT-I activates ESCRT-III, which is responsible for the concentration of MVBs' cargo molecules using the ESCRT-II complex or ALIX, and the ESCRT-III/VPS4 complex triggers the constriction and induces the cleavage of the vesicle buds during abscission (meaning the act of cutting off). Afterwards, MVBs interact with a specific combination of soluble N-ethylmaleimide-sensitive factor attachment protein receptors (SNAREs) within the plasma membrane, and mammalian-derived exosomes are secreted to the extracellular milieu of the cell [Id., citing Kalluri, R. and LeBleu, VS, Science (2020) 367 (6478): eaau6977; Vietri, M. et al. Nat. Rev. Mol. Cell Biol. (2019) 21 (1): 25-42].

Depending on the particular composition of their exosomal proteins, the fate and function of mammalian-derived exosomes in their biogenesis, cargo selection, targeting ability, and endocytosis under both physiological and pathological conditions can vary. Among the protein cargoes of mammalian-derived exosomes, those proteins participating in cell adhesion (e.g., integrin, lactadherin, ICAM), intracellular trafficking (e.g., RAB GTPases, annexin), signal transduction (e.g., protein kinases, 0-catenin, 14-3-3, G proteins), biogenesis factors (e.g., ALIX, TSG101, syntenin, ubiquitin, clathrin, VPS32, VPS4), and as well as chaperones (e.g., HSP70, HSP90), have been evaluated [Id., citing van Niel, G. et al. Nat. Rev. Mol. Cell Biol. (2018) 19: 213-228].

Generally, mammalian-derived exosomes are characterized by the presence of specific lipids such as phosphatidylserine, cholesterol, sphingomyelins, and ceramides, which are responsible for intercellular signaling as well as being essential in structural stability. In addition, they may also carry genetic material such as messenger RNA (mRNA), microRNAs (miRNAs), and non-coding RNAs. Overall, mammalian-derived exosomes are not only considered to be effective intercellular transporters of proteins, lipids, and nucleic acids, but also novel regulators that can alter the physiological and pathological functions of both recipient and parent cells via their various components.

Although, the application of mammalian derived exosomes is promising, several major issues limit the clinical use of these exosomes, including (1) their low production yield, (2) the time-consuming and laborious production processes, and (3) the difficulties involved with achieving high-quality and uniform exosomes [Id, citing [Li, P. et al. Theranostics (2017) 7(3): 789-804]; van den Boom, J G et al. Nat. Biotechnol. (2011) 29 (4): 325-326; Lobb, R J et al. J. Extracell. Vesicles (2015) 4 (1): 27031; Vader, P. et al. Adv. Drug Deliv. Rev. (2016) 106 (Part A): 148-156; van Deun, J. et al. J. Extracell. Vesicles (2014) 3 (1): 24858].

### Plant-Derived Exosome-Like Nanovesicles (PELNVs)

Evidence of the existence of plant extracellular vesicles dates back to 1967 [Halperin, W. and Jensen, WA. J. Ultrastruct. Res. (1967) 18: 428-443]. Notwithstanding that many aspects of plant-derived exosome-like nanovesicles are not fully understood, they may provide therapeutic advantages compared with mammalian-derived exosomes or artificial nanoparticles, including facile large-scale production [Subha, D. et al. Discover Nano 92023) 18: 146, citing Li, Z. et al. Sci. Rep. (2018) 8(1): 14644], low toxicity, reduced immunogenicity [Id, citing Deng, Z. et al. Mol. Ther. (2017) 25 (7): 1641-1654], efficient cellular uptake [Id, citing Wang, Q. et al. Nat. Commun. (2013) 4: 1867] and high biocompatibility and stability [Id, citing Zhang, M. et al. Biomaterials (2016) 101: 321-340].

WO2020/180311 entitled “Plant-based exosome compositions and use thereof for rejuvenating skin” describes isolation of exosomes from leaf flesh of a plant from the Asphodelaceae family, more particularly of the Aloe genus, including Aloe vera, Aloe barbadensis Miller, Aloe aborenscens, or Aloe vera L. conditioned by growing the plant under conditions that included a heat shock of the plant at a temperature of from about 33 °C to about 45 °C for about 1 hour to about 3 hours. The level of heat shock stress response molecule HSP70 in the heat-shocked plant exosome was from about 10 times to about 20 times higher than the level of heat shock stress response molecule in the exosomes from non-heat-shocked plants.

Plant-derived exosome-like nanovesicle (PELNV) preparation has been attempted using many common edible plants, such as grapefruit, grape, lemon, broccoli, carrot, apple, coconut, and ginger [Kim, J. et al. Asian J. of Pharmaceutical Sci. (2022) 17: 53-69, citing Ju, S. et al. Mol. Ther. (2013) 21 (7): 1345-1357; Wang, B. et al. Mol. Ther. (2014) 22 (3): 522-534; Wang, Q. et al. Cancer Res. (2015) 75(12): 2520-2529; Brahmbhatt, M. et al. Nutr. Cancer (2013) 65 (2): 263-272; Raimondo, S. et al. Oncotarget (2015) 6 (23): 19514-19527; Deng, Z. et al. Mol. Ther. (2017) 25 (7): 1641-1654; Mu, J. et al. Mol. Nutr. Food Res. (2014) 58 (7): 1561-1573; Zhao, Z. et al. J. Agric. Food Chem. (2018) 66 (11): 2749-2757; Yu, S. et al. Food Chem. (2019) 272: 372-378; Fujita, D. et al. Mol. Pharm. (2018) 15 (12): 5772-5780; Zhang, M. et al. Mol. Ther. (2016) 24 (10) 1783-1796]. PENLV are known to be similar to mammalian-derived exosomes in terms of such properties as size distribution, surface electric charge, morphology, density, and certain components [Id., citing Ju, S. et al. Mol. Ther. (2013) 21 (7): 1345-1357; Raimondo, S. et al. Oncotarget (2015): 19514 19514-27; Deng, Z. et al. Mol. Ther. (2017) 25 (7): 1641-1654]. Like mammalian-derived exosomes, PELNVs also comprise biomolecules, such as RNAs, proteins, and lipids, that regulate physiological processes [Id., citing Teng, Y. et al. Cell Host Microbe (2018) 24 (5): 637-652].

Despite many similarities between PELNVs and mammalian-derived exosomes, the two groups of vesicles show some differences. First, the lipid bilayer of mammalian-derived exosomes are mainly composed of cholesterol, glycosphingolipids, ceramides and phosphatidylserine, which provide stability and a unique rigidity [Id., citing Stemersch, S. et al. J. Control Release (2016) 244: 167-83; Nishio, M. et al. Bioscens. Bioelectron (2020) 150: 111918; Mashouri, L. et al. Mol Cancer (2019) 18: 75; Waldenstrom, A. and Ronquist, G. Circ. Res. (2014) 114 (2): 315-324]. In contrast, the exosomal membranes of PELNVs are enriched with phosphatidic acid (PA), phosphatidylcholines (PC), digalactosyldiacylglycerol (DGDG), and monogalactosyldiacylglycerol (MGDG) [Id., citing Teng, Y. et al. Cell Host Microbe (2018) 24 (5): 637-652], which provide inherent mammalian-cell-regulating activities in the intestinal microenvironment.

Second, there is no canonical ESCRT-0 complex in higher plants; instead, TOM1-like (TOL) proteins with conserved VHS (VPS27, HRS, STAM) domains, which are considered to have a general membrane targeting/cargo recognition role in vesicular trafficking [see Lohi, O. et al. FEBS Lett. (2002) 513 (1): 19-23] act as substitutions for ESCRT-0 as ubiquitin binding proteins and play a role in the vacuolar sorting of the auxin efflux facilitator PIN-FORMED 2 (PIN2) in the early endosome [Kim, J. et al. Asian J. of Pharmaceutical Sci. (2022) 17: 53-69, citing Gao, C. et al. Trends Plant Sci. (2017) 22 (11): 986-998]. The cargo is subsequently transported to the ESCRT-1 and ESCRT-II complexes via the ubiquitin-binding proteins, the ESCRT-II complex then stimulates and recruits ESCRT-III through an interaction between VPS25 and VSP20. The ESCRT-III complex constricts the plasma membrane and cleaves the necks of the buds that form on the cytosolic face to release ILVs containing cargo into the endosome [Id., citing Yanez-Mo, M. et al. J. Extracell. Vesicles (2015) 4: 27066; Cui, Y. et al. Mol. Plant (2016) 9 (6): 774-786].

Third, surface markers to differentiate between types of extracellular vesicles in plants are yet to be identified, and the mechanism underlying trafficking of the EVs is unknown.[Subha, D. et al. Discover Nano (2023) 18: 146].

Fourth, each PELNV has different characteristics and components depending on the cell of origin. For example, Zhuang et al. reported that a 6-shogaol (one of the main bioactive principles in dried ginger rhizomes) rich in ginger-derived exosome-like nanoparticles (GDENs) activates Nrf2 by regulating TLR4/TRIF pathway, protecting against alcohol-induced liver damage through the anti-inflammatory actions of this pathway [Id., citing Zhang, X. et al. J. Extracell. Vesicles (2015) 4 (1): 28713]. Ju et al. reported that grape exosome-like nanoparticles induced the recovery of intestinal stem cells through the Wnt/0-catenin signaling pathway, which regulates genes including AXIN-2 (a tumor suppressor protein that negatively regulates the WNT signaling pathway), Cyclin D1 (a protein that controls the transition from G1 to S phase in the cell cycle), c-MYC (a widely acting transcription regulatory factor), and EGF (a growth factor that stimulates the proliferation, differentiation, and survival of epithelial and epidermal cells) [Id., citing Ju, S. et al. Mol Ther. (2013) 21 (7): 1345-1357]. In terms of the cellular uptake of grape-derived exosome-like nanoparticles (GELNs), GELNs showed a high selectivity to intestinal stem cells and were significantly inhibited by an inhibitor of cytochalasin-D (a mycotoxin that has the ability to bind to actin filaments and block polymerization and the elongation of actin) known as micropinocytosis inhibitor. In contrast, a clathrin-mediated endocytosis inhibitor did not affect their uptake [Id.].

Fifth, beneficially, unlike mammals, plants do not harbor zoonotic or human pathogens.

Plants produce EVs in response to numerous biotic and abiotic environmental stresses, including pathogen infection and attack [Kim, J. et al. Asian J. of Pharmaceutical Sci. (2022) 17: 53-69, citing An, Q. et al. New Phytol. (2006) 172 (3): 563-576; An, Q. et al. Cell Microbiol. (2006) 8 (6): 1009-1019]. Qianli et al., who observed the proliferation of intravacuolar MVBs in the cytoplasm and the structural perturbation of the organelle and its membrane by trafficking in barley leaf cells, provided evidence of the generation and secretion of plant-derived exosome-like nanovesicles [Id., citing Qianli, A J V B, and Huckelhoven, R. Plant Signal Behav. (2007) 2 (1): 4-7]. Additionally, it has been reported that enhancement of fungal infections induces the proliferation of MVBs at the site of infection by stimulating plant innate immune responses in plant defense [Id., An, Q. et al. New Phytol. (2006) 172 (3): 563-576; Wang, F. et al. PLoS Pathog. (2014) 10 (7): e1004243].

Evidence indicates that plant extracellular vesicles play a multifaceted role in plant defense [Subha, D. et al. Discover Nano 92023 18: 146]. They function as mobile pockets highly enriched with antimicrobials and plant-defense proteins which initiate the plant immune response at the site of pathogen entry. For example, defense-related proteins involved in the myrosinase-glycosinolate system such as penetration resistance protein 3 (PEN3), which provides cell wall defense against pathogens, the nitrate sensor NRT1, the myrosinase epithiospecifier modifier 1 (ESM), and reactive oxygen species (ROS) signaling proteins were identified in the EV proteome [Id. citing Rutter, B D and Innes, R W. Plant Physiol. (2017) 173: 728-41; Rutter, B D, and Innes, R W. Curr. Opin. Plant Biol. (2018) 44: 16-22].

Plant EVs also can hold cell wall remodeling enzymes. On sensing an infection, the plant tries to strengthen its defense by improving cell wall integrity, while the pathogen takes advantage of the cell wall metabolism in the host to establish the infection [Id., citing Vincent, D. et al. Front. Plant Sci. (2020) 10: 1626]. Cell wall remodeling proteins are transported through EVs in several cases. Proteomic studies on EVs from sunflower seedlings revealed that 47% of the identified proteins are cell wall-associated including the enzymes in-charge of polysaccharide reorganization [Id., citing Regente, M. et al. Exp. Bot. (2017) 68: 5485-5495].

When the proteome of the plant EVs from Arabidopsis was analyzed, a total of 93 RNA binding proteins (RBPs) were recognized. Those with ssRNA binding capacity are AGO1, DEAD-box RNA helicase RH11, RH37, RH52, Annexin 1 (ANN1) and annexin 2 (ANN2). These proteins were also easily detected by western blotting in isolated EVs thereby confirming the proteome results. These RBPs co-localize with the tetrospanin8 (TET8) marker, and the EV associated small RNAs previously identified (such as TAS1c-SiR483, TAS2-SiR 453 and miR166) were also found associated with this subgroup of exosome-like EVs. The trans-acting siRNA (TAS1) are secondary small interfering RNAs synthesized from noncoding transcripts that are targeted by miRNAs, playing a role in regulating gene expression, such as the patterning of floral structures [Gan, E-S et al. Intl Review of Cell & Molecular Biol. (2013) 305: 115-161]. The TAS1 family is composed of three genes (TAS1a, TAS1b, TAS1c) and the ta-siRNAs they encode (e.g., siR255/siR480) target four mRNAs encoding proteins of unknown function. The three miRNAs that guide TAS mRNA cleavage are miR173, miR390 and miR828 [Choudhuri, S. Biochem. Biophys. Res. Communic. (2009) 388 (2): 177-180]. EV localized RBPs specifically bind with the small RNAs and aid in their selective loading into the EVs. The Annexins (ANN1 and ANN2), which were also found in the EVs do not bind with ssRNAs specifically; it has been suggested that they might contribute to the stabilization of the RNA inside the vesicles [Subha, D. et al. Discover Nano 92023 18: 146].

### Plant Heat Shock Proteins Counter Plant Biotic and Abiotic Stresses

Plants continuously confront harsh environments like high/low temperatures, salinity, drought, light stresses, flooding, physical wounding and chemical pollutants (e.g., heavy metals), which produce secondary stresses, such as osmotic and oxidative stresses, [al Haq, S. et al. Intl J. Molec. Sci. (2019) 20: 5321, citing Swindell, W R et al. BMC Genom. (2007) 8: 125; Al-Whaibi, M H. J. King Saud Univ. Sci. (2011) 23: 139-150; Guo, M. et al. Front. Plant Sci. (2016) 7: 114; Xu, Y. et al. Int. J. Proteom. (2011) 2011: 529648].

Biotic stresses like pathogens (e.g., viruses, bacteria, and fungi) and pests (e.g., nematodes, insects, and rodents) also restrict plant productivity [Id., citing Dodds, P. and Rathjen, J. Nat. Rev. Genet. (2010) 11: 539; Li, R. et al. PLoS One (2015) 10: e0143261; Jones, J T et al. Mol. Plant Pathol. (2013) 14: 946-561; Rybicki, EP. Arch. Virol. (2015) 160: 17-20; Gorovits, R. et al. Cell Stress Chaperones (2017) 22: 345-355]. Negative effects of these stresses on plant germination [Id., citing Cheng, L. et al. J. Integr. Plant Biol. (2009) 51: 489-499], include stunted growth [Id, citing Srivastava, S. et al. J. Environ. Biol. (2012) 33: 657; Wahid, A. et al. Environ. Exp. Bot. (2007) 61: 199-223], sunburn and scorching of leaves [Id, citing Rodruguez, M. et al. Plants Biotecnologia (2005) 22: 1-10], loss of photosynthetic pigment, decreased production of photo-assimilates, and depletion of carbohydrate reserves which results in starvation [Id, citing Tan, W. et al. J. Plant Physiol. (2011) 168: 2063-2071; Jiang, C. et al. Plant Cell Environ. (2009) 32: 1046-1059; Demirevska-Kepova, K. et al. Biol. Plant (2005) 49: 521-525; Djanaguiraman, M. et al. J. Agron. Crop Sci. (2009) 195: 213-224].

Abiotic stresses also negatively affect the reproductive characteristics of plants by enhancing male sterility [Id, citing Young, L W et al. J. Exp. Biol. (2004) 55: 485-495] and increasing premature flower and fruit drop [Id, citing Tubiello, F N et al. Proc. Natl Acad. Sci. USA (2007) 104: 19686-19690] which results in significant low yield and quality. It has been reported that an increase in temperature by 1 °C results in a 4-10% yield decrease [Id, citing Wang, X. et al. J. Cereal Sci. (2012) 55: 331-6]. As a consequence of these stresses, reactive oxygen species (ROS) are produced which lead to oxidative stress and, ultimately, results in cell death. ROS could be singlet oxygen (¹O₂), superoxide radical (O₂·—), hydrogen peroxide (H₂O₂) and hydroxyl radical (OH—), which are produced in cell organelles such as mitochondria, peroxisomes and chloroplasts in oxidative stress situations and react with all types of macromolecules like pigments, proteins, lipids and DNA [Id., citing Karuppanapandian, T. et al. Plant Physiol. Biochem. (2011) 49: 168-177; Moller, I M et al. Anu. Rev. Plant Biol. (2007) 58: 459-481].

Plants respond morphologically to elevated temperature and light stress by changing their leaf orientation [Id, citing Wahid, A. et al. Environ. Exp. Biol. (2007) 61: 199-223], anatomically by altering stomatal conductance and increased leaf pubescence [Id, citing Zhang, J. et al. Field Crop Res. (2006) 97: 111-119; Banon, S. et al. Sci. Hortic. (Amst.) 2004 101: 333-342], and phenologically (a branch of science dealing with the relations between climate and periodic biological phenomena) by shifting and improvising the developmental stages to escape the abiotic stress condition [Id, citing Sato, S. et al. Ann. Bot. (2006) 97: 731-8]. Plants also change their metabolic processes and physiology to retain root hydraulic conductance [Id., citing Morales, D. et al. Biol. Plant (2003) 47: 203], accumulation of compatible osmolytes, such as sugars, sugar alcohols, proline and phenolic compounds under saline and water-logged conditions, as well as high temperature and water deficit conditions [Id, citing Wahid, A. and Close, TJ. Biol. Plant (2007) 51: 104-109].

Moreover, plants manage to maintain photosynthetic machinery [Id, citing Salvucci, M E et al. Physiol. Plant (2004) 120: 179-186] by changing their assimilate partitioning (e.g., a shift occurs from symplastic, where the symplast pathway involves protoplasts that are found within the living cytoplasm cells, to apoplastic (a non-living route since the water solution moves in the spaces between cells and along the cell wall) [Id, citing Wahid, A. and Ghazanfar, A. J. Plant Physiol. (2006) 163: 723-730]. During the onset of the stress situations, plants also improvise the hormonal balance of abscisic acid (ABA), ethylene, and salicylic acid (SA) as a signaling molecule in the systemic acquired resistance. Similarly, jasmonic acid (JA) and other steroids enhance stress tolerance and resistance [Id, citing Wang, LJ and Li, SH. Plant Sci. (2006) 170: 685-694].

Furthermore, secondary metabolites, such as isopropanoid, carotenoid, flavonoid, anthocyanin, lignin, and isoprenoids [Id., citing Wahid, A. and Ghazanfar, A. J. Plant Physiol. (2006) 163: 723-730; Sharkey, TD. Plant Cell Environ. (2005) 28: 269-277], also are produced and accumulated. Most studies indicate that plant responses to two or more factors are unique and differ from the response to one factor only [Al-Whabi, MH, J. King Saud University-Science, J. King Saud Univ. —Science (2011) 23: 139-150].

Besides these adaptations, plants also have sophisticated adaptive systems at the cellular and molecular levels. During the onset of stress, plants reduce the synthesis of normal protein production and transcribe and translate heat shock proteins (HSPs). Added to transcriptional regulations, plants also have some sophisticated post-transcriptional modifications which help the plant to cope with these stresses, such as alternative splicing and micro RNA (miRNA). Alternative splicing, which generates multiple copies from a single gene, helps the plants to mitigate abiotic stresses [al Haq, S. et al. Intl J. Molec. Sci. (2019) 20: 5321, citing Laloum, T. et al. Trends Plant Sci. (2018) 23: 140-150]. An important plant post-transcriptional modification strategy is miRNA, which binds to the mRNA at any point to repress translation or direct cleavage of the mRNA. Some plant miRNAs also are involved with abiotic stress tolerance [Id., citing Zhang, B. et al. Dev. Biol. (2006) 289: 3-16].

HSPs are proteins characterized by the presence of a carboxylic terminal called heat-shock domain [Al-Whaibi, M H., J. King Saud Univ. —Science (2011) 23: 139-150, citing Helm, K W et al. Mol. Cell Biol. (1993) 13: 238-247]. The concentration of HSPs dramatically increases when cells are grown at higher temperatures where HSPs help newly synthesized proteins to fold or protect proteins that might misfold and thereby lose their potential functional conformation during a stress event, such as biotic and/or abiotic stress [al Haq, S. et al. Intl J. Molec. Sci. (2019) 20: 5321, citing Weinmann, H. and Ottow, E. In Comprehensive Medicinal Chemistry II, Taylor, J B and Triggle, D J Eds. Elsevier: Oxford UK (2007); pp. 221-251]. These stress-responsive biomolecules act as molecular chaperones which perform under stress situations [Id., citing Ahuja, I. et al. Trends Plant Sci. (2010) 15: 664-674]. The main functions of HSPs are proper protein folding, unfolding and transport, in conjunction with their localization in the cell, and, subsequently, disposal and degradation of the non-native proteins, [Id., citing Balchin, D. et al. Science (2016) 353: acc4353; Hartl, F U et al. Nature (2011) 475: 324-332; Benesova, M. et al. PLoS ONE (2012) 7: e38017].

HSPs are important in the plant life cycle as their role extends beyond the protection from biotic and abiotic stresses. Although HSPs (with the exception of ubiquitin) were first characterized due to their response to high temperatures, now many HSPs are found in normal, non-stressed cells, and are produced at particular stages of the cell cycle, or during development in the absence of stress [Id., citing Vierling, E. Annu Rev. Plant Biol. (1991) 42: 579-620].

For example, beside stress-responsive biomolecules, HSPs also are involved in plant growth and development under normal conditions, like the flowers, seeds and fruits set, development [Id., citing Eck, E R H B et al. J. Biosci. (2007) 32: 501-510], tuberization [Id., citing Lehesranta, S J et al. Proteomics (2006) 6: 6042-6052; Agrawal, L. et al. J. Proteome Res. (2013) 12: 4904-4930; Agrawal, L. et al. J. Proteome Rs. (2008) 7: 3803-17; Ahn, Y J and Zimmerman, J L. Plant Cell Environ. (2006) 29: 95-104,] and nutrient uptake [Id., citing Shekhar, S. et al. J. Proteom. (2016) 143: 306-17]. HSPs are found in different compartments of the cells, such as cytoplasm, nucleus, and cell organelles, e.g., mitochondria, chloroplasts and endoplasmic reticulum [Id., citing Water, E R et al. J. Exp. Biol. (1996) 325-338; Boston, R S et al. In Post-Transcriptional Control of Gene Expression in Plants, Springer: Berlin, Germany (1996), pp. 191-222].

HSP expression is controlled by transcription factors known as heat shock factors (HSF). Among the HSF classes, HSFA positively regulates plant tolerance to anoxia, heat, osmotic and oxidative stresses [Id., citing Zhuang, L. et al. Intl J. Mol. Sci. (2018) 19: 2702]. HSFA1, found in tomato plants, is considered a master regulator of signal perception, transduction and controlling the expression of stress-responsive genes, including HSPs [Id., citing Guo, M. et al. Front Plant Sci. (2016) 7: 114; Mishra, S K et al. Genes Dev. (2002) 16: 1555-1567] and other stress responsive genes. HSFs play an important and significant role in modifying physiological and biochemical processes, which leads to the development of tolerance to stresses [Id., citing Kotak, S. et al. Curr. Opin. Plant Biol. (2007) 10: 310-316; Scharf, K D et al. Biophys. Acta Gene Regul. Mech. (2012) 1819: 104-119]. HSPs show a response to biotic and abiotic stress situations by up- or down-regulation, but, sensing signals and transduction, particularly in biotic stress, remains to be explored [Id., citing Singh, R K et al. Sci. Rep. (2016) 6: 32641].

### Heat Shock Proteins

Heat shock proteins (HSPs) are conserved in almost all organisms from bacteria, to fungi, plants and animals, including human beings. HSPs are classified and named based on the molecular weight in kilo Dalton (kDa), which ranges from 8-200 kDa [Id]. Based on molecular weight, HSPs generally are classified into the following sub-families: HSP100, HSP90, HSP70, HSP60, and small HSPs (sHsps), which are characterized by a conserved sequence of 80-100 amino acid residues, with a molecular weight ranging from 13 to 43 kDa [Reddy, V S et al. Cell Stress and Chaperones (2018) 23: 441-454; al Haq, S. et al. Intl J. Molec. Sci. (2019) 20: 5321, citing Schoffi, F. et al. Sci. Research (1999) 81-98; Schlesinger, MJ. J. Biol. Chem. (1990) 265: 12111-4; Kotak, S. et al. Plant Cell (2007) 19: 182-195; Shamovsky, I. and Nudler, E. Cell Mol. Life Sci. (2008) 65: 855-861; Guo, M. et al. Frong. Plant Sci. (2015) 6: 806]. These chaperone families are involved in maintaining cell homeostasis, transportation of newly synthesized proteins across cell organelles, and folding-preventing misfolded, denatured and aggregated proteins caused by stress conditions [Id., citing Balchin, D. et al. Science (2016) 353: acc4353; Ratajczak, E. et al. J. Mol. Biol. (2009) 386: 178-89; Tyedmers, J. et al. Nat. Rev. Mol. Cell Biol. (2010) 11: 777-788].

The genes which encode different HSPs are found in different cell compartments, such as the nucleus, mitochondria, chloroplast, endoplasmic reticulum and cytosol [Id, citing Liu, D. et al. Plant Physiol. Biochem. (2006) 44: 380-386]. Similarly, the accumulation of these HSPs in different parts of the cell also depends on the intensity of the stress. Nuclear HSPs, for instance, are accumulating in the cytosol at the lower and higher temperatures of 27 °C and 43 °C, respectively, while the same aggregate in chloroplast is at 37 °C [Id, citing Waters, E R et al., J. Exp. Bot. (1996) 47: 325-338].

Different HSPs are found and differentially expressed in different species and in different genotypes but in the same species, as investigated by Korotaeva et al., (2001) and Nieto-Sotelo et al., (2002) [Korotaeva, N E et al. Russ. J. Plant Physiol. (2001) 48: 798-803; Nieto-Sotelo, J. Plant Cell (2002) 14: 1621-33] in small HSPs where five sHSPs showed a response to a higher temperature (42 °C) in maize but only one is expressed in wheat and rice. Likewise, HSP68 is expressed in mitochondria under stress situations in potatoes, tomatoes, and soybeans [Id, citing Neumann, D. et al. Planta (1993) 190: 32-43].

The most studied species of plant is Arabidopsis thaliana, where the response to heat-shock treatment occurs through the participation of a number of Hsps: 13 Hsp20; 8 Hsp70; 7 Hsp90, 8 Hsp100, and 21 transcription factors (Hsfs) [Al-Whaibi, MH, J. King Saud University—Science (2011) 23: 139-150, citing Swindell, W R et al, BMC Genomics (2007) 8: 125], but in tomato there are at least 15 Hsfs [Id, citing von Koskull-Doring, P. et al Trends Plant Sci. (2007) 12: 452-457].

Higher plants are characterized by the presence of at least 20 types of sHsps. sHsps are usually undetectable in plant cells under physiological conditions, but are induced upon stress and plant tolerance to stress, including drought, salinity, oxidized species, and low temperatures [Id, citing Low, D. et al Planta (2000) 211: 575-582; Hamilton, E V and Heckathorn, SA Plant Physiol. (2001) 126: 1266-1274; Scharf, K D et al Cell Stress Chaperones (2001)6: 225-237; Zhang, J-H et al. Scientia Horticulturae (2008) 117: 231-240].

Furthermore, the sHsps of A. thaliana and Lycopersicon esculenium are divided into three subclasses [Id, citing Scharf, K D et al Cell Stress Chaperones (2001) 5: 225-237; Siddique, M et al, Cell Stress Chaperones (2003) 8: 381-394]. These included: subclass Cl, represented by 6 proteins in A. thaliana and five proteins in L. esculenium; subclass C11 represented by two genes in both plants; and subclass CIII represented by one gene in both plants. A study reported the presence of other groups in the cytoplasm of A. thaliana cells, and could be categorized into subclasses: CIV, CV, CVI, and CII. Each subclass has its own distinct characteristics and role.

There are six groups of genes that encode for the sHsps. The grouping is based on the sequence similarity and the location of these proteins in the cell. There are two classes of proteins (Class I and Class II) in the cytoplasm encoded by two groups of genes. Other locations are chloroplasts, endoplasmic reticulum, mitochondria and membranes [Id., citing Vierling, E. Annu. Rev. Plant Phys. (1991) 42: 579-620; Waters, E R et al. H. Exp. Bot. (1996) 47: 325-338]. The expression of genes for these sHsps is limited in the absence of environmental stress and occurs in some stages of growth and development of plants, such as embryogenesis, germination, development of pollen grains, and fruit ripening [Id., citing Sun, W. et al. Biochim Biophys. Acta (2002) 1577: 1-9].

Some HSPs showed a tissue-specific response to stress situations; HSP101 was expressed more in reproductive parts like tassels, ear, and endosperm, than in vegetative parts like leaves and roots in maize [al Haq, S. et al. Intl J. Molec. Sci. (2019) 20: 5321, citing Young, T E et al. Plant Physiol. (2001) 127: 777-791]. Some HSPs responded differently to the varying length of stresses. As reported by Heckathorn et al. [Id., citing Heckathorn, S A et al. Int. J. Plant Sci. (1998) 159: 39-45], HSP45, a nuclear protein, accumulated in the chloroplast after a 3 h exposure to heat stress, which returned to its native state after removal of stress. Similarly, HSPs are triggered differently at different development stages. HSP45, for example, showed a response in the whole plant to a stress situation, while HSP64 and HSP72 only showed expression in the reproductive parts i.e., pollens [Id., citing Frova, C. et al. Dev. Genet. (1989) 10: 324-332; Ristic, Z. et al. J. Plant Physiol. (1996) 149: 424-432].

### Regulation of HSPs

When plants are exposed to stress, the synthesis of normal proteins is decreased while the expression of special genes is up-regulated, and, as a result, synthesis of HSPs is triggered. The transcriptional regulation of HSPs to respond to stresses is called the heat shock response (HSR) [Id., citing Shamovsky, I., Nudler, E. Cell Mol. Life Sci. (2008) 65: 855-861].

The transcription of HSPs is controlled by regulatory proteins called heat stress transcription factors (HSFs), which are located in the cytoplasm in an inactive state. HSFs are considered as transcriptional activators for heat shock [Al-Whaibi, M. J. King Saud Univ. -Science (2011) 23: 139-150, citing Clos, J. et al Cell (1990) 63: 1085-1097; Baniwal, S K et al. J. Biosci. (2004) 29: 471-487; Hu, W. et al Plant Sci. (2009) 176: 583-590]. Each factor has one carboxylic terminal (C-terminal) and three amino terminals (N-terminal) and has the amino acid leucine [Id, citing Schuetz, T J et al. Proc. Natl Acad Sci. USA (1991) 88: 6911-15].

Plants are characterized by a large number of transcriptional factors, at least 21 [Id, citing Nover L. and Baniwal, SK. In Intl Symposium on Environmental Factors, Cellular Stress and Evolution, Varanasi, India, October 13-15 (2006), p. 15]. The factors have been classified into three classes (HsfA, HsfB, and HsfC) according to the structural differences in their aggregation in triples (oligomerization domains). While each factor has its role in the regulatory network, all cooperate in regulating many functions and different stages of response to periodic heat stress (triggering, maintenance, and recovery).

The HSR is regulated by the heat stress transcription factors (HSFs) in the promoter region, which bind to cis-acting elements known as HSE (Heat Shock Elements) [al Haq, S. et al. Intl J. Molec. Sci. (2019) 20: 5321, citing Akerfelt, M. et al. Nat. Rev. Mol. Cell Biol. (2010) 11: 545-552; Pirkkala, L. et al. FAEB J. (2001) 15: 1118-1131]. HSFs are classified as three types: HSFA, HSFB, and HSFC; the functions of these classes vary from each other. Among the HSFs, HSFA, which is found in the cytosol in a monomeric state, regulates the HSPs cycle. The activity of the HSFA, under normal conditions, is regulated negatively by HSP90s, which are checked in the form of phosphoproteins [Id, citing Ali, A. et al. Mol. Cell Biol. (1998) 18: 4949-4960].

During the onset of stress, this repression is reversed and HSP90 dissociates and changes into a functional trimer state. The HSFA homo-trimer then binds to the Heat Shock Element (HSE) in the promoter region [Sou, J. et al. Cell (1998) 94: 471-480], transcription occurs and HSPs are synthesized [Id, citing Calderwood, S K et al. Sign. Transduct. Insights (2010) 2: 13-24]. Among the HSFAs, HSFA1 acts as the master regulator in tomatoes [Id., citing Mishra, S K et al. Genes Dev. (2002) 16: 1555-67]. HSFA2 is structurally and functionally the same as HSFA1 but is expressed only in stressed plants. Under stress situations, HSFA2 forms a super activator hetero-oligomer structure with HSFA1, which is more efficient than the individual HSFs, which not only regulate the down-stream stress related HSP genes, but also the protective enzyme genes such as GST, GR, POX and APX [Id, citing Zhuang, L. et al. Int. J. Mol. Sci. (2018) 19: 2702; Scharf, K D et al. Biochim. Biophys. Acta Gene Regul. Mech. (2012) 1819: 104-119].

Some studies also report that HSP gene expression positively regulates protective enzyme activities. In Arabidopsis, for example, overexpression of HSP17.8 enhanced superoxide dismutase (SOD) activity and, in tobacco, HSP16.9 increased the activities of ROS scavenging by ROS scavenger gene expression, e.g., peroxidase (POD), catalase (CAT) and SOD [Id., citing Driedonks, N. et al. Front. Plant Sci. (2015) 6: 999].

Post-transcriptional modification, such as alternative splicing, also regulate the HSFs. HSFA2 under heat stress, for instance, binds to its own promoter region and activates its own transcription in a positive auto-regulatory loop. Similarly, HSFA is regulated by dehydration-responsive element-binding protein 2 (DREB2) under stress, which in turn regulates the stress related genes in many plants [Id., citing Laloum, T. et al. Trends Plant Sci. (2018) 23: 140-150] Similarly, miRNAs also play a vital role in the stress response by down-regulation of stress-related genes. For example, some miRNAs are reported to exert positive regulation in drought, cold, salinity, hormones and nutrient starvation stresses, such as miR159, miR319, miR395, and miR402 [Id., citing Zhang, B. et al. Dev. Biol. (2006) 289: 3-16]. Conversely, in Arabidopsis short term heat stress, miR398 negatively regulates the expression of cold shock domain (CSD)1, CSD2 and capsanthin-capsorubin synthase (CCS), which yield SOD [Id., citing Driedonks, N. et al. Front. Plant Sci. (2015) 6: 999].

### Biotic Stress Tolerance

Plant growth, development, yield, and quality are affected adversely by several biotic factors such as pathogenic bacteria, fungi, viruses, and nematodes. Biotic factors directly deprive their host plants of their nutrients, which result in reduced plant vigor, growth, productivity and sometimes leads to death of the host plants. Biotic stresses are a major cause of pre- and post-harvest losses.

Animals have an immune system, which helps them to adapt to biotic stresses such as new diseases and possess immunological memory so that they can respond more rapidly and effectively to the past pathogens. Although plants lack this adaptive immune system, they have evolved several sophisticated strategies to counteract these biotic stresses. These defense mechanisms are stored in the plant's genome at the genetic level, which encode thousands of stress resistance genes. HSP response to biotic stresses depend on the nature of the causal organisms and plant genotypes, either susceptible or resistant, and the developmental stage [Id., citing Dodds, P. and Rathjen, J. Nat. Rev. Genet. (2010) 11: 539].

### Abiotic Stress Tolerance

Abiotic stresses are extreme environmental conditions like extreme temperatures, water deficit, and ion imbalance due to heavy metals and salinity, which pose a serious threat to plants survival, yield and quality.

### High Temperature Stress

Several studies have indicated that many high molecular weight HSPs showed a response under high-temperature stress, such as HSP118, HSP114, HSP110, HSP108, HSP104, HSP103, HSP101, HSP100 and HSP97, respectively, [Id., citing Young, T E et al. Plant Physiol. (2001) 127: 777-91, Singla, S L et al. J. Biosci. (1998) 23: 337-45]. Among the HSP100 class, a significant high temperature response (HTR) was shown by HSP101 [Id., citing Lee, U. et al. Plant J. (2007) 49: 115-127] and also was involved in thermo-tolerance in Arabidopsis [Id., citing Queitsch, C. et al. (2000) 12: 479-492]. It was confirmed further in maize that HSP101 was involved in thermo-tolerance [Id., citing Nieto-Sotelo, J. et al. Plant Cell (2002) 14: 1621-33]. Merret et al., (2017) and Mcloughlin et al., (2016) [Id., citing Merret, R. et al. Plant Physiol. (2017) 174: 1216-1225; McLoughlin, F. et al. Plant Physiol. (2016) 172: 1221-36] confirmed the role of HSP101 in thermotolerance in Arabidopsis but also established that this played a role in recovery after heat shock.

Besides the role of HSP101 as a chaperone, HSP100 also was involved in development [Id., citing Pyatrikas, D V et al. Russ. J. Plant Physiol. (2014) 61: 80-80]. Low molecular weight HSPs i.e., HSP18.1 and HSP17.9, accumulated in the pea while it was treated for four hours at 42 °C. The response and expression of HSPs also were development stage and different tissue specific. In maize subjected to 40 °C, HSPs (~101, -70 and -17.6) were induced. Above 36 °C, fertilization was reduced, although HSPs were induced in female reproductive parts but, when studied, mature pollens were more sensitive to heat stress [Id., citing Dupuis, I., and Dumas, C. Plant Physiol. (1990) 94: 665-670]. In contrast, HSP70s were expressed more in tomato pollens [Id., citing Duck, N B and Folk, W R. Plant Mol. Biol. (1994) 26]: 1031-1039]. In Arabidopsis HSP70 was expressed more in mitochondria under high temperature stress [Id., citing Sung, D Y et al. Plant Physiol. (2001) 126: 789-800]. Chloroplast HSP70.1, 70.2 and mitochondrial HSP22 also were involved in seed development besides its role as a chaperone [Id, citing Su, PH and Li, H. Plant Physiol. (2008) 146: 1231-1241; Avelange-Macherel, MH. Plant Cell Environ. (2015) 38: 1299-1311].

HSP90 also showed increased expression under heat stress situations. HSP90 has been reported in rice and Arabidopsis [Id, citing Hu, W. et al. Plant Sci. (2009) 176: 583-590; Prasad, B D et al. PLoS ONE (2010) 5: e12761] and all classes of HSP90 (A, B, and C) in soybeans [Id, citing Xu, J. et al. PLoS ONE (2013) 8: e69810]. Under normal conditions, HSP90 negatively regulated HSFs and kept the regulation of all HSPs checked [Id, citing Yamada, K. et al. J. Biol. Chem. (2007) 282: 37794-37804].

HSP70s and HSP60s chaperonin families are the most studied of the chaperones under heat stress, which maintained protein proper folding using ATPs [Id., citing Hartl, F U et al. Nature (2011) 475: 324-332]. Cytosolic HSP70 was involved in heat stress tolerance in Arabidopsis [Id., citing Jungkunz, I. et al. Plant J. (2011) 66: 983-995]. HSP70s have been studied under high temperature stress in a variety of plant crops, such as witch-grass and alfalfa [Id., citing Song, G. et al. Plant Cell Resp. (2018) 37: 1485-1497; Li, Z. et al. J. Plant Res. (107) 130: 387-96]; vegetables like pepper, tomato, cabbage, potato; ornamental plants like chrysanthemum [Id., citing Guo, M. et al. Plant Si. (2016) 252: 246-256; Huang, L. et al. Protoplasma (2019) 256: 39-51; Usman, M G, et al. Cell Stress Chaperones (2018) 23: 223-234; Zhang, S. et al. Plant Cell Physiol. (2017) 59: 58-71; Lee, SS. Et al. Curr. Genom. (2010) 19: 12-20; Liu, J. et al. Sci. Rep. (2018) 8: 16628; Zhang, Y. et al. Plant Omics (2014) 7: 229]; grain such as wheat [Id., citing Id, H W et al. J. Mol Sci. (2018) 19: 1594]; and tea [Id., citing Chen, J. et al. Int. J. Mol. Sci. (2018) 19: 2633]. Xu et al., (2010) [Id., citing Xu, C. and Huang, B. Crop Sci. (2010) 50: 2543-2552] observed the expression pattern of chloroplast HSP60, not only in normal conditions but also under high-temperature and drought situations. It was responsible for ribulose biphosphate carboxylase/oxygenase (Rubisco, a key enzyme in photosynthesis) assembly, protection and also chloroplast development.

Low molecular weight HSPs such as the HSP10, HSP20 and HSP40 families were up-regulated under high-temperature stress situations in various plant crops [Id., citing Xu, Y. et al. Intl J. Proteomics (2011) 2011: 529648; Huang, L. et al. Protoplasma (2019) 256: 39-51; Kumar, N. et al. 3 Biotech (2017) 7: 205; Liao, J L et al. J. Exp. Bot. (2013) 65: 655-671; Lin, C J et al. J. Agric. Food Chem. (2010) 58: 10545-10552; Majoul, T. et al. Proteomics (2004) 4: 505-13; Wang, X. et al. Genes Genom. (2018) 11: 1-8]. Some small HSPs were also genotype-specific and were up-regulated in resistant cultured varieties bred by humans (“cultivars”), such as foxtail millet, while some small HSPs were down-regulated in sensitive genotypes [Id., citing Singh, R K et al. Sci. Rep. (1016) 6: 32641]. Some co-chaperones were involved in thermo-tolerance as HSP40. Correlation of small HSPs with HSP100, HSP70 and HSP60 suggested their role as holders in disaggregation and protein folding [Zhang, Y. et al. Plant Omics (2014) 7: 229].

### Low-Temperature Stress

Cold stress affects plant enzymes, membrane plasticity, changes physiology and metabolism, and sometimes causes water starvation and desiccation, which creates a stress condition for the plant that adversely affects plant growth, development and yield. Low temperature also is associated with protein disfunction and denaturing, which induce the accumulation of HSPs [Id. citing Hashimoto, M. and Komatsu, S. Proteomics (2007) 7: 1293-1202; Hlavackova, I. et al. Int. J. Mol. Sci. (2013) 14: 8000-8024; Kosova, K. et al. J. Proteom. (2011) 74: 1301-1322]. Many HSPs responded to cold stress and were up-regulated in Arabidopsis, tobacco, maize, rapeseed, chicory, poplar, wheat and barley [Id., citing Hlavackova, I. wt al. Intl J. Mol Sci. (2013) 14: 8000-8024; Bae, M S et al. Plant J. (2003) 36: 652-63; Jin, Y. et al. Afr. J. Biotechnol. (2011) 10: 18991-19004; Kollipara, K P et a 1. Plant Physiol. (2002) 129: 974-992; Reddy, R K et al. lant Sci. (1998) 131: 131-37; Degand, H. et al. Proteomics (2009) 9: 2903-2907; Ghosh, D. and Xu, J. Front. Plant Sci. (2014) 5: 6; Vitamvas, P. et al. Proteomics (2012) 12: 68-85; Ono, K. et al. Plant Sci. (2001) 160: 455-61]. Under low-temperature stress situations, HSPs were induced and translocated into various cell organelles to protect them from cold stress [Id., citing Bae, M S et al. Plant J. (2003) 36: 652-663].

Bae et al., (2003) investigated this in Arabidopsis treated with cold stress at 40 °C for 6 h. HSP70s were up-regulated and their traffic from the cytoplasm to the nucleus was observed. A similar event was observed in the pea mitochondria when treated at 4 °C for 36 h [Id., citing Taylor, N L et al. Mol. Cell Proteom. (2005) 4: 1122-1133]. Some HSPs accumulated in tissue specifically upon low-temperature exposure, as in poplar, where HSPs were accumulated in leaves [Id., citing Renaut, J. et al. Plant Biol. (2004) 6: 81-90]. Regarding rice, low-temperature stress and a gradual decrease in the temperature from 15 °C to 0 °C, with an interval of 5 °C, up-regulated HSP95 and HSP75, and HSP70 accumulated in the chloroplast, as this was the part of the plant vulnerable to low temperature [Id, citing Hahn, M. and Walbot, V. Plant Physiol. (1989) 91: 930-938; cui, S. et al. Proteomics (2005) 5: 3162-3172].

Some of the HSPs, like HSP90 in wheat and HSP60 and HSP21 in sunflowers, are down-regulated in response to cold stress [Id, citing Vitamvas, P. et al. Proteomix (2012) 12: 68-65; Balbuena, T S et al. J. Proteome Res. (2011) 10: 2330-2346]. A similar trend also was reported by Hlavackova et al., (2013) and Rinalducci et al., (2011) [Id, citing Hlavackova, I. et al. Intl J. Mol. Sci. (2013) 14: 8000-8024; Rinalducci, S. et al. J. Proteom. (2011) 74: 643-659] where Rubisco stability was associated with down-regulation of HSP60 and HSP21 in winter wheat.

### Drought Stress

Drought stress, in combination with other abiotic stresses such as high light and temperature stress, negatively affects plant morphological, physiological and molecular characteristics, which leads to lowered photosynthesis, hormonal imbalance, mineral nutrient starvation and an ultimate oxidative stress [Id, citing Komatsu, S. et al. J. Proteome Res. (2011) 10: 3993-4004]. Removal of water disrupts the normal structure of the lipid bilayer plasma membrane, which results in the displacement of membrane proteins, denaturation of membrane-based enzymes and, as a result, membrane permeability, physiology and metabolism are adversely affected [Id, citing Salehi-Lisar, SY and Bakhshayeshan-Agdam, H. Drought Stress in Plants: Causes, Consequences, and Tolerance, Physiology and Biochemistry; Springer International Publishing: Cham, Switzerland; Berlin, Germany, 2016; pp. 1-16; Chaves, M M et al. Funct. Plant Biol. (2003) 30: 239-264]. Dehydration stress also affects the quantity and quality of normal plant proteins and, as a result, stress related proteins including HSPs are induced. For example, HSP70 was up-regulated in drought stress in the seedling of upland rice [Id, citing Reddy, P S et al. PLoS ONE (2014) 9: e89125].

Similarly, transgenic Arabidopsis and sugarcane also showed HSP up-regulation and demonstrated drought tolerance [Id, citing Yer, E N et al. Gene (2018) 678: 324-36; Subba, P. et al. J. Proteome Res. (2013) 12: 5025-5047]. The expression pattern of HSPs is also genotype-specific; Burke J J et al., [Id, citing Burke, J J et al. Plant Physiol. (1985) 78: 394-398] studied combined drought and heat stress in irrigated and non-irrigated cotton, where more HSPs accumulated in non-irrigated cotton. Maize heat tolerant and sensitive cultivars were studied under high temperature and dehydration situations, where HSP accumulation was more in drought stress conditions [Id., citing Ramanjulu, S. and Bartels, D. Plant Cell Environ. (2002) 25: 141-151]. The same was demonstrated by Benesova et al., [Id., citing Bensevova, M. et al. PLoS One (2012) 7: e38017], where HSP70 and HSP26 were induced in drought-stressed maize. A study on chickpea HSP70 reported that HSPs were first down-regulated in the early stage of growth in drought-tolerant cultivars (meaning a plant that has been produced in cultivation by selective breeding).

In contrast, HSPs were abundant in drought-sensitive cultivars, which indicated that HSPs responded to drought not only in the specific genotypes but, also, during the developmental stage. Similarly, small HSPs expressed highly in drought-tolerant cultivars as compared to those that were sensitive in chickpea [Id., citing Subba, P. et al. J. Proteome Res. (2013) 12: 5025-5047]. The same trend also was observed in poplar and Kentucky bluegrass [Id., citing Xu, C. and Huang, B. Crop Sci. (2010) 50: 2543-2552; Burke, J J et al. Plant Physiol. (195) 78: 78: 394-398]. HSP17.7 showed drought tolerance in transgenic rice, and other HSPs also were involved in the acclimation of bryophytes (nonvascular plants, e.g., mosses) to drought stress [Id., citing Agrawal, L. et al. Front. Plant Sci. (2016) 7: 1466; Ristic, Z. et al. Plant Physiol. (1991) 97: 1430-1434]. Proteomics studies revealed that nuclear and HSPs in the extracellular matrix were both up-regulated to drought stress [Id., citing Cruz de Carvalho, R. et al. Plant Cell Environ. (2014) 37: 1499-1515; Pandey, A. et al. Mol. Cell Proteom. (2008) 7: 88-107; Pandey, A. et al. J. Proteom Res. (2010) 3443-64; Bhushan, D. et al. J. Proteome Res. (2011) 10: 2027-2046].

### Salinity Stress

Studies show that many HSPs are induced and up-regulated in saline stress situations like HSP70 in rice seedlings [Id., citing Ngara, R. et al. Proteomics (2014) 14: 611-621], wheat [Sobhanian, H. et al. J. Proteo. (2011) 74: 1323-37], and poplar HSP70-9, -12 and -33 [Id., citing Manaa, A. et al. J. Exp. Bot. (2011) 62: 2797-2813]. Furthermore, HSP40 in rice [Id., citing Wang, X. et al. Genes Genom. (2018) 11: 1-8] and poplar, HSP100-21 and -75), HSP90-9 and -12), HSP60-31, -33, -38 and -49), HSP40-113 and -117, and HSP21 were also up-regulated under salt stress [Id., citing Manaa, A. et al. J. Exp. Bot. (2011) 62: 2797-2813]. In wheat hybrid Jinan 177 and its salt-resistant hybrid, protein profiling showed HSPs and chaperones were induced highly under salt stress [Id., citing Wang, M. et al. Proteomics (2008) 8: 1470-89].

HSPs were studied in relation to programmed cell death (PCD) in a rice root at higher salt, where mitochondrial HSP70 were the up-regulated proteins that possibly were involved in PCD regulation [Id., citing Han, F. et al. Biochim. Biophys. Acta Proteins Proteom. (2009) 1794: 1625-1634]. Soybean proteomic studies showed a differential HSPs expression of HSP90, chloroplast HSP70, HSP60 and HSP20 under salt stress [Id., citing Song, H. et al. Plant Mol. Biol. Report (2009) 27: 342-349]. Different HSPs in Arabidopsis like HSP90 [Id., citing Xu, J. et al. PLoS ONE (2013) 8: e69810; Choudhary, M K, et al. Mol Cell Proteom. (2009) 8: 1579-98], HSP100, Clp (B1, B2), Clp (D1, D2) and small HSPs in rice [Id., citing Muthusamy, S K et al. Front. Plant Sci. (2016) 7: 929; Song, H. et al. Planta (2009) 229: 955-64] showed tolerance to high salinity stress. The role of HSPs in response to salinity stress is also genotype-specific, as recorded in soybean, where HSPs were induced more in salt resistant cultivars [Id., citing Pi, E. et al. Mol. Cell Proteom. (2016) 15: 266-288].

### Light Stress

As autotrophs (meaning an organism that is able to form nutritional organic substances from simple inorganic substances such as carbon dioxide), plants require light for photosynthesis. Excess light damages the photosynthetic apparatus, and plants undergo a phenomenon known as photorespiration. During this process, toxic chemicals, rather than sugars, along with ROS, are produced. These toxic chemicals in the chloroplast can damage the photosystem II permanently by excessive absorption of light [Id., citing Timperio, A M et al. J. Proteom. (2008) 71: 391-411; Kumar, M. et al. Front. Plant Sci. (2017) 7: 2023]. Rossel et al., [Id. citing Rossel, J B et al. Plant Physiol. (2002) 130: 1109-1120] reported that many HSPs were up-regulated upon high light stress (HLS) in Arabidopsis.

A similar over-accumulation of nuclear HSP70 was observed in Chlamydomonas. The thylakoid proteome analysis of Arabidopsis was studied with respect to high light saturation involving isoforms of chloroplast HSP70 along with the accumulation of other osmolytes like anthocyanins and ascorbates. In the marine ecosystem where low light created a stress, HSP70, ClpB1, Sti, and HSP60 were up-regulated [Id., citing Kumar, M. et al. Front. Plant Sci. (2017) 7: 2023]. Under high light saturation, small HSP23 was seen to be involved in the post-transcriptional regulation in a Chenopodium rubrum cell suspension [Id., citing Debel, K. et al. Planta 91997](201(3): 326-33).

### Chemical Pollutant Stress

Plant productivity is restricted by chemical pollutants in the soil media, such as heavy metals. These pollutants affect plant growth either by displacement of essential cations from specific binding sites or by generation of oxidative stress by the generation of ROS [Al HaQ. S. et al. Intl J. Molec. Sci. (201 20: 5321, citing Sharma, SS. And Detz, K J. Trends Plant Sci. (2009) 14: 43-50).

HSPs can be induced by heavy metal stress. For example, HSP70s were differentially expressed and accumulated in the roots of tomatoes [Id., citing Rodriguez-Celma, J. et al. J. Proteom. (2010) 73: 1694-1706]. Similarly, the HSP70 sub-family, DnaK (Bip), was up-regulated in rice seedlings [Id., citing Ahsan, N. et al. C. R. Biol. (2007) 330: 735-468]. Arabidopsis exposure to cadmium stress induced many HSPs [Id., citing Sarry, J. et al. Proteomics (2006) 6: 2180-2198]. Similarly, increased expression was reported for HSP80 and HSP17.9 in rice [Id., citing Ahsan, N. et al. C R Biol. (2007) 330: 735-746], HSP 90s in Lotus corniculatus [Id., citing Nacascues, J. et al. New Phytol. (2012) 193: 625-636], HSP17.7 in carrots and HSP26 in soybeans [Id., citing Czmecka, E et al. Mol. Cell Biol. (1988) 8: 1113-1133] under cadmium, lead and arsenic stresses.

Using a comparative proteomic analysis of poplar under cadmium stress, a differential expression pattern of HSP was noted. Similarly, in soybeans, two-folds higher accumulation of HSP was recorded in Cd-accumulating genotypes, while there was less HSP70 expression in lower Cd-accumulating varieties, which showed that HSP expression was also genotype-specific [Id., citing Hossain, Z. et al. Amino Acids (2012) 43: 2393-2416]. When flax was cultured on heavy metal treated media, many heavy metal binding proteins, including HSP70 accumulation, were enhanced, while HSP83 showed down-regulation. HSP90.3 enhanced cadmium stress tolerance by lowering germination potential in Arabidopsis, mediating the antioxidant enzymes [Id., citing Song, H M et al. Biol. Plant (2012) 56: 197-199].

### Flooding Stress

Waterlogging/flooding is also an environmentally limiting factor that hinders plant growth and development. A gradual decrease of redox potential and oxygen in the soil are the ill effects of flooding [Id., citing Hossain, Z. et al. J. Plant Physiol. (2009) 166: 1391-1404]. Studies show that HSPs are involved in plant resistance against flooding stress by up-regulation and higher gene expression, which is organ-specific. As noticed by Chen et al., [Id., citing Chen, Y. et al. Proteome Sci. (2014) 12: 33] in the soybean plasma membrane where HSP70 accumulated more than 10 fold, this occurrence occurred more in cotyledon than the roots of the soybean.

Discussed in another proteomic study by the same group of researchers, HSP60 was differentially regulated in soybeans [Id., citing Komatsu, S. et al. PLoS ONE (2013) 8: e65301]. In contrast, HSPs were induced in flooding stress, but were not mandatory for resistance in flooding stress and were genotype specific. As for resistant and susceptible cultivars of rice to anoxia and hypoxia conditions, HSPs were more up-regulated in the sensitive cultivars than resistant genotypes [Id., citing Komatsu, S. et al. J. Proteome Res. (2011) 10: 3993-4004]. Proteomics study of flooding stress in relation to PCD in maize revealed that HSP70s were up-regulated [Salehi-Lisar, S. Y.; Bakhshayeshan-Agdam, H. Drought Stress in Plants: Causes, Consequences, and Tolerance, Physiology and Biochemistry; Springer International Publishing: Cham, Switzerland; Berlin, Germany, 2016; pp. 1-16]. The same pattern of flooding tolerance was studied in rice protoplast where ectopic mtHSP70 expression protected H₂O₂ induced PCD [Id., citing Komatsu, S. et al. J. Proteome Res. (2011) 10: 3993-4004]. Similarly, in Arabidopsis anoxia tolerance was enhanced via HSFA2-mediated production of HSP70 and HSP101.

### Oxidative Combined Stress

Since plants are exposed to many stresses simultaneously, such as light, this creates high temperature stress that leads to dehydration. Such situations lead to oxidative or secondary stress and plants have to adjust their signaling pathways and metabolism to ensure their growth and development [Id., citing Mittler, R. et al. Trends Biochem. Sci. (2012) 37: 118-125; Ngara, R. et al. Proteomics (2014) 14: 611-621; Kilian, J. et al. Plant J. (2007) 50: 347-363; Scarpeci, T E et al. Plant Signal Behav. (2008) 3: 856-857]. Oxidative stress generates reactive oxygen species (ROS) which, in high concentrations, are harmful to cellular structures. HSPs respond to multiple stress situations and enable the plants to cope with the challenging environment.

For example, HSP70 expression was higher in tobacco exposed to heat stress but was even higher to the combined stress of heat and drought [Id., citing Rizhsky, L. et al. Plant Physiol. (2002) 130: 1143-1151]. Ectopic expression of genes from soybeans in Arabidopsis GmHSP90 showed tolerance to heat, salinity and osmotic stresses, although response in salinity was not as high as that to combined stresses [Id., citing Xu, J. et al. PLoS ONE (2013) 8: e69810]. A similar pattern was observed with small HSPs in rice to multiple stresses [Id., citing Zou, J. et al. J. Plant Physiol. (2012) 169: 628-635; Want, A. et al. Plant Breed (2015) 134: 384-393]. Overexpression of HSP17.6 in Arabidopsis enhanced tolerance to salinity combined with dehydration, but no response was noted to high temperature stress only [Id., citing Sun, W. et al. Plant J. 2001]27: 407-15].

Single or combined stresses led to the production of ROS and oxidative stress which, if not checked timely, are very detrimental to plants [Id., citing Hossain, Z. et al. J. Plant Physiol. (2009) 166: 1391-1404]. Under oxidative stress, overexpression of organelle and cytosolic HSP90 enhanced tolerance in Arabidopsis. Similar results were reported by Nishizawa-Yokoi et al. [Nishizawa-Yokoi, A. et al. Plant Cell Physiol. (2010) 51: 486-496], where HSP90 regulated HSFA2, which enhanced tolerance to oxidative stress. Queitsch et al. [Id., citing Queitsch, C. et al. Plant Cell (2000) 12: 479-492] reported oxidative stress accumulated HSP100/Clp B, ClpC2 and ClpD1 in rice. HSPs protected vital cellular parts under oxidative stress, as demonstrated by Downs, C A et al. [Downs, C A et al. J. Plant Physiol. (1999) 155: 488-496], where small HSPs protected the photosystem II from oxidative stress and photo-inhibition. Different organelle HSPs also responded to oxidative stress. Microtubule-associated mtHSP22 accumulation was enhanced in tomatoes under oxidative stress [Id., citing Banzet, N. et al. Plant J. (1998) 13: 519-577]. Small HSPs responded to oxidative stress, as HSP16.4 and HSP17 accumulated in multiple stress situations in Arabidopsis and carrots, respectively [Id., citing Jiang, C. et al. Plant Cell Environ. (2009) 32: 1046-1059; Sarry, J. et al. Proteomics (2006) 6: 2180-98].

### Genetic Engineering Induction Studies

Al-Whaibi et al [J. King Saud Univ—Science (2011) 23: 39-50] describe a series of field tests to modulate induction of HSPs in plants. In general, the expression of HSPs and their factors Hsfs was induced largely by heat, cold, salinity and osmotic stresses; they emphasized the importance of studying stress combinations to end up with tolerant plants. The response to other stress factors depends on protein class and tissues. For example, under all types of stresses, high expression response for class HSP20 was recorded. Wounding of the roots of the plant stimulated (after 12 h) the expression of several genes from classes HSP20; HSP70 and HSP100. High response of expression of genes for HSPs and Hsfs occurred under UV-B stress in aerial tissues (shoot), but there was no expression in non-aerial tissues [Id., citing Sindell, W R et al. BMC Genomics (2007) 8: 125].

The response of plants to heat shock results in changes in the level of enzymes, cellular membrane structure, photosynthesis activity and protein metabolism [Id., citing Singla, S L et al. In: Prasad, M N V (Ed), Plant Ecophysiology, John Wiley, New York, (1997) pp. 101-127]. It has been reported that high temperature changed the properties of membranes of the nucleus, ER, mitochondria and chloroplasts of the rice plant O. saiiva [Id., citing Pareek, A. et al. J. Biosci. (1998) 23: 361-367].

Scientists have modified plant cells to show an increase in cold stress tolerance by increasing gene expression of glycerol 3-phosphate acyltransferase from Cucurbita maxima and A. thaliana in tobacco plant cells, resulting in an increase in the degree of unsaturation of the lipids in the thylakoid membranes of the chloroplast. Therefore, increasing the degree of unsaturation of fatty acids leads to an increase in cold tolerance.

In an attempt to increase salinity tolerance of the wheat plant, one report disclosed that transgenic plants that contained a gene (CtHSR1) from the yeast Candida tropicalis were subjected to water stress, high salinity and heat stresses under operating greenhouse conditions and in the field. Stress conditions were withholding watering the plant for two weeks (water stress), watering in the presence of 400 mM NaCl (salinity stress), and subjecting the plants to 46 °C for 2 h followed by a 3 day period recovery at 28 °C (heat stress). The results showed improvement of growth under both drought and heat stresses and lesser but still significant to salinity stress [Id., citing Blumwald, E. and Arif, A.].

miRNAs in Plants: Their Role in Abiotic Stresses

A growing body of research has demonstrated that miRNAs act on target genes and are involved in various biological functions of plants. Small RNA high-throughput sequencing has been widely used to identify and functionally analyze miRNAs in plants [Zhang, F. et al. Front. Plant Sci. (2022) 13: 919243, citing Sunkar, R. et al. Plant Cell (2005) 17: 1397-1411; Fahlgren, N. et al. PLoS One (2007) 2: e219 et al.; Creighton, C J et al., Brief Bioinform. (2009) 10: 490-497]. According to the records registered in miRBase (mirbase.org), 38,589 hairpin precursors and 48,860 mature microRNAs have been identified through experimental or computational approaches from 271 organisms, including more than 70 plants, such as Arabidopsis thaliana (326 precursors, 428 mature), Oryza sativa (604 precursors, 738 mature), Zea mays (174 precursors, 325 mature), Triticum aestivum (122 precursors, 125 mature), Glycine max (684 precursors, 756 mature), Solanum tuberosum (224 precursors, 343 mature), Nicotiana tabacum (162 precursors, 164 mature), Solanum lycopersicum (112 precursors, 147 mature), Gossypium raimondii (296 precursors, 296 mature), Medicago truncatula (672 precursors, 756 mature), Populus trichocarpa (352 precursors, 401 mature), Sorghum bicolor (205 precursors, 241 mature), Brassica napus (90 precursors, 92 mature), Vitis vinifera (163 precursors, 186 mature), and so on [Id.].

Squamosa Promoter-Binding Protein-Like (SPL) genes encode plant-specific transcription factors that play important roles in plant phase transition, flower and fruit development, plant architecture, gibberellins signaling, sporogenesis, and response to copper and fungal toxins [Chen, X. et al. J. Integr. Plant Biol. (2010) 52 (11): 946-951]. The Squamosa promoter binding protein (SBP)-box proteins are plant-specific transcriptional factors in plants [Abdulllah, M. et al. Front. Genet. (2018) 9: 64]. MYB proteins are key factors in regulatory networks controlling development, metabolism and responses to biotic and abiotic stresses [Dubos, C. et al. Trends in PlantSci. (2010) 15 (10): P573-P581].

A preliminary statistical analysis showed that several miRNAs (miR156, miR159, miR160, miR164, and miR172) are the most studied miRNA families that showed diverse roles in diverse stresses and diverse plant species. MiR156-targeted Squamosa promoter binding protein like (SPL) transcription factors (TFs) modulate plant architecture, including grain size, panicle branching, and higher grain productivity in rice [Id., citing Jiao, Y. et al. Nat. Genet. (2010) 42: 541-544; Miura, K. et al. Nat. Genet. (2010) 42: 545-549; Wang S. et al., Nat. Genet. (2012) 44: 950-954] and modulate plant architecture and tuberization in potato and temporal regulation of shoot development in Arabidopsis thaliana [Id., citing Gruber, A J and Zavolan, M. Epigenomics (2013) 5: 671-83; Yao, Q. et al. Curr. Opin. Chem. Biol. (2019) 51: 11-17]. The miRNA156-targeted SPL/SBP (Squamosa promoter binding protein)-box protein transcription factors (TFs) regulate tomato ovary and fruit development [Id., citing Ferreira e Silva, G F et al., Plant J. (2014) 78: 604-618]. The miRNA159 post-transcriptionallly suppresses MYB TFs in the regulation of programmed cell death in Arabidopsis [Id., citing Alonso-Peral, M M et al., Plant Physiol. (2010) 154: 757-771], floral development and stem elongation in rice [Tsuji, H. et al. Plant J. (2006) 47: 427-444], anther development and heat response in wheat [Id., citing Wang Y. et al., PLoS ONE (2012) 7: e48445], leaf and floral development in tomato [Id., citing Buxdorf, K. et al. Planta (2010) 232: 1009-1022], and targeting of isotrichodermin C-15 hydroxylase and its involvement in immune response of cotton [Id, citing Zhang, T. et al. Nat. Plants (2016) 2: 16153].

The miR160 regulates a group of repressor auxin response factors (ARFs), which are mainly involved in auxin hypersensitivity and regulation of floral organ development, seed germination, and post-germination stages in Arabidopsis thaliana [Id, citing Liu, PP> et al. Plant J. (2007) 52: 133-146; Liu, X. et al. Plant J. (2010) 62: 416-428], ovary patterning, floral organ abscission and lamina outgrowth in tomato [Id, citing Turner, M. et al. Plant Physiol. (2013) 162: 2042-2055], growth and developmental defects of rice [Id, citing Huang, J. et al. Sci. Repts (2016) 6: 29938], and inhibition of symbiotic nodule development in soybean [Id, citing Turner, M. et al. Plant Physiol. (2013) 162: 2042-55].

The miR164-directed cleavage of NACI mRNA affects lateral root development in Arabidopsis [Id, citing Guo, H S et al., Plant Cell (2005) 17: 1376-1386], maize [Id., citing Li, J. et al., BMC Plant Biol. (2012) 12: 220], drought resistance in rice [Id., citing Fang, Y. et al. J. Exp. Bot. (2014) 65: 2119-2135], and boundary specification in tomato [Id., citing Berger, Y. et al. Development (2009) 136: 823-832] and negatively regulates the resistance of wheat to stripe rust [Id., citing Turner, M. et al. Plant Physiol. (2013) 162: 2042-2055]. miR172 suppressing AP2 genes induce flowering, spikelet determinacy (which determines whether a spikelet, the basic unit of a cereal influorescence, produces a fixed number of florets or continues producing new florets over time), and floral organ abnormalities in rice (Id., citing Zhu, H. et al., BMC Genomics (2009) 20: 33; Lee, Y S et al. Rice (2014) 7: 31), promote vegetative phase change in maize [Id., citing Lauter, N. et al. Proc. Natl Acad. Sci. USA (2005) 102: 9412-9417], regulate soybean nodulation [Id., citing Yan, Z. et al. Mol. Plant Microbe Interact. (2013) 26: 1371-1377], and affect cleistogamous flowering in barley [Id., citing Nair, S K et al. Proc. Natl Acad. Sci. USA (2010) 107: 490-495] and graft-transmissible induction of potato tuberization [Id., citing Martin, A. et al. Development (2009) 136: 2873-2881].

The expression patterns of miR159, miR160, miR166, miR396, miR393, etc. have significant alterations in drought and salt stress, which suggests that these miRNAs play a vital role in abiotic stress alleviation in chickpea [Id., citing Jatan, R. et al., Genomics (2019) 111: 509-519; Jatan, R. et al. Environ. Exp. Bot. (2019) 157: 217-227]. Moreover, miR164a-CUC1, miR167-NRAMP1, miR393a-5p-TIR1, and miR396a-5p-GRF1 modules might be involved in the regulation of root, leaf, and flower development of Arabidopsis during Pseudomonas putida-inoculation [Id., citing Jatan, R. et al., Int. J. Mol. Sci. (2020) 21: 5468].

Plant miRNAs also are implicated in abiotic stress response mechanisms with regard to oxidative stress and effects on DNA in different plant species [Id., citing Pagano, L. et al., Environ. Exp. Bot. (2021) 184: 104369]. miRNAs play a key role in responding to unfavorable conditions, such as low temperature stress [Id., citing Aslam, M. et al. Int. J. Mol. Sci. (2020) 21: 8441], high temperature stress [Id., citing Zhang, M. et al. Intl J. Mol Sci. (2019) 20: 1754]; drought stress [Id., citing Ni, Z. et al. Biochem. Biophys. Res. Commun. (2012) 427: 330-35], salt stress [Id., citing Nguyen, D Q et al. Intl J. Mol. Sci. (2020) 21: 7879]; and heavy metal stress [Id., citing Ding, Y. et al. J. Agric. Food Chem. (2020) 68: 1958-1965] through regulating expression of related target genes in plants.

### Anatomy and Physiology of the Skin

The skin is the largest organ in the body, consists of several layers, plays an important role in biologic homeostasis, and is comprised of the epidermis and the dermis. The epidermis, which is composed of several layers beginning with the stratum corneum, is the outermost layer of the skin, and the innermost skin layer is the deep dermis. The skin has multiple functions, including thermal regulation, metabolic function (vitamin D metabolism), and immune functions. FIG. 1 presents a schematic diagram of the anatomy of the skin.

In humans, the usual thickness of the skin is from 1-2 mm, although there is considerable variation in different parts of the body. The relative proportions of the epidermis and dermis also vary, and a thick skin is found in regions where there is a thickening of either or both layers. For example, on the interscapular (between the shoulder blades) region of the back, where the dermis is particularly thick, the skin may be more than 5 mm thick, whereas on the eyelids it may be less than 0.5 mm. Generally, the skin is thicker on the dorsal or extensor surfaces of the body than on the ventral or flexor surfaces; however, this is not the case for the hands and feet. The skin of the palms and soles is thicker than on any dorsal surface except the intrascapular region. The palms and soles have a characteristically thickened epidermis, in addition to a thick dermis.

The entire skin surface is traversed by numerous fine furrows, which run in definite directions and cross each other to bound small rhomboid or rectangular fields. These furrows correspond to similar ones on the surface of the dermis so that, in section, the boundary line between epidermis and dermis appears wavy. On the thick skin of the palms and soles, the fields form long, narrow ridges separated by parallel coursing furrows, and in the fingertips, these ridges are arranged in the complicated loops, whorls (verticil) and spirals that give the fingerprints characteristic for each individual. These ridges are more prominent in those regions where the epidermis is thickest.

Where there is an epidermal ridge externally there is a corresponding narrower projection, called a “rete peg,” on the dermal surface. Dermal papillae on either side of each rete peg project irregularly into the epidermis. In the palms and soles, and other sensitive parts of the skin, the dermal papillae are numerous, tall and often branched, and vary in height (from 0.05 mm to 0.2 mm). Where mechanical demands are slight and the epidermis is thinner, such as on the abdomen and face, the papillae are low and fewer in number.

### Epidermis

The epidermis provides the body's buffer zone against the environment. It provides protection from trauma, excludes toxins and microbial organisms, and provides a semi-permeable membrane, keeping vital body fluids within the protective envelope. Traditionally, the epidermis has been divided into several layers, of which two represent the most significant ones physiologically (FIG. 2). The basal-cell layer, or germinative layer, is of importance because it is the primary source of regenerative cells. In the process of wound healing, this is the area that undergoes mitosis in most instances. The upper epidermis, including stratum and granular layer, is the other area of formation of the normal epidermal-barrier function.

### Stratum Corneum and the Acid Mantle

Stratum corneum is an avascular, multilayer structure that functions as a barrier to the environment and prevents transepidermal water loss. Studies have shown that enzymatic activity is involved in the formation of an acid mantle in the stratum corneum. Together, the acid mantle and stratum corneum make the skin less permeable to water and other polar compounds and indirectly protect the skin from invasion by microorganisms. Normal surface skin pH is between 4 and 6.5 in healthy people; it varies according to area of skin on the body. This low pH forms an acid mantle that enhances the skin barrier function.

### Other Layers of the Epidermis

Other layers of the epidermis below the stratum corneum include the stratum lucidum, stratum granulosum, stratum germinativum, and stratum basale. Each contains living cells with specialized functions (FIG. 2). For example, melanin, which is produced by melanocytes in the epidermis, is responsible for the color of the skin. Langerhans cells are involved in immune processing.

### Dermal Appendages

Dermal appendages, which include hair follicles, sebaceous and sweat glands, fingernails, and toenails, originate in the epidermis and protrude into the dermis hair follicles and sebaceous and sweat glands, contribute epithelial cells for rapid re-epithelialization of wounds that do not penetrate through the dermis (termed partial-thickness wounds). The sebaceous glands are responsible for secretions that lubricate the skin, keeping it soft and flexible. They are most numerous in the face and sparse in the palm of the hands and soles of the feet.

Sweat gland secretions control skin pH to prevent dermal infections. The sweat glands, dermal blood vessels, and small muscles in the skin (responsible for goose pimples) control temperature on the surface of the body. Nerve endings in the skin include receptors for pain, touch, heat, and cold. Loss of these nerve endings increases the risk for skin breakdown by decreasing the tolerance of the tissue to external forces.

The basement membrane both separates and connects the epidermis and dermis. When epidermal cells in the basement membrane divide, one cell remains, and the other migrates through the granular layer to the surface stratum corneum. At the surface, the cell dies and forms keratin. Dry keratin on the surface is called scale. Hyperkeratosis (thickened layers of keratin) is found often on the heels and indicates loss of sebaceous gland and sweat gland functions if the patient is diabetic. The basement membrane atrophies with aging; separation between the basement membrane and dermis is one cause for skin tears in the elderly.

### Dermis

The dermis, or the true skin, is a vascular structure that supports and nourishes the epidermis. In addition, there are sensory nerve endings in the dermis that transmit signals regarding pain, pressure, heat, and cold. The dermis is divided into two layers: the superficial dermis and the deep dermis.

The superficial dermis consists of extracellular matrix (collagen, elastin, and ground substances) and contains blood vessels, lymphatics, epithelial cells, connective tissue, muscle, fat, and nerve tissue. The vascular supply of the dermis is responsible for nourishing the epidermis and regulating body temperature. Fibroblasts are responsible for producing the collagen and elastin components of the skin that give it turgor. Fibronectin and hyaluronic acid are secreted by the fibroblasts. The structural integrity of the dermis plays a role in the normal function and youthful appearance of the skin.

The deep dermis is located over the subcutaneous fat; it contains larger networks of blood vessels and collagen fibers to provide tensile strength. It also consists of fibroelastic connective tissue, which is yellow and composed mainly of collagen. Fibroblasts are also present in this tissue layer. The well-vascularized dermis withstands pressure for longer periods of time than subcutaneous tissue or muscle. The collagen in the skin gives the skin its toughness. Dermal wounds, e.g., cracks or pustules, involve the epidermis, basal membrane, and dermis. Typically, dermal injuries heal rapidly.

### Hair

Hair is a filamentous outgrowth of protein found only on mammals. The hair of non-human mammal species is commonly referred to as fur. In some species, hair is absent at certain stages of life.

Hair grows from hair follicles deep in the dermis and projects from the epidermis of the skin.

Human skin has two types of hair: vellus hair and terminal hair. Much of human hair is short, under-pigmented vellus hair rather than terminal hair. The most noticeable part of human hair is the hair on the head, which can grow longer than on most mammals and is denser than most hair found elsewhere on the body. The term “scalp” refers to the integument of the upper part of the head, usually including the associated subcutaneous structures. The scalp is the anatomical area bordered by the face anteriorly and the neck to the sides and posteriorly. A healthy scalp is characterized by clean, hydrated skin and good blood circulation, balanced oil production, and the absence of inflammation, itching and flaking.

Vellus hair is short, fine, “peach fuzz” body hair. It is a very soft, generally pale, and short hair that grows in most places on the human body in both sexes. It is usually less than two cm long and the follicles are not connected to sebaceous glands. It is most easily observed in women and children, as they have less terminal hair to obscure it. It is also found in pre-adolescents and in male pattern baldness.

Terminal hair is developed hair, which is generally longer, coarser, thicker and darker than the shorter and finer vellus hair. Phases of growth in terminal hair are more apparent than in vellus hair; it generally has a longer anagen phase. It has associated sebaceous glands, whereas a vellus hair may not. Under certain conditions, such as puberty, some vellus hair may become androgenic hair. Under other conditions, such as male pattern baldness, it may revert to a vellus-like state.

Each hair comprises two structures: the follicle in the skin and the shaft we see. The follicle contains several layers. At the base of the follicle is a projection called a papilla, which contains capillaries, or tiny blood vessels, that feed the cells. The dermal papilla consists of an egg-shaped accumulation of mesenchymal stem cells (MSCs) surrounded by ground substance that is rich in acid mucopolysaccharides (AMPs).

The living part of the hair, the area surrounding the papilla called the bulb, is the only part fed by the capillaries. The bulb encompasses the dermal papilla and the hair matrix. The hair matrix surrounds the top and sides of the dermal papilla. It is the actively growing portion of the follicle, which consists of a collection of epidermal cells that rapidly divide, move upward, and give rise to the hair shaft and the internal root sheath. The cells in the bulb divide every 23 to 72 hours, faster than any other cells in the body.

The hair follicle contains stem cells from different developmental origins, such as epithelial stem cells, melanocyte stem cells, and mesenchymal stem cells (MSCs) [Wang, B. et al. World J. Stem Cells (2020) 12(6): 462-470, citing Kiani, M T et al. ACS Biomater. Sci. Eng. (2018) 4: 1193-1207]. These stem cells continuously self-renew, differentiate, regulate hair follicle development, and contribute to hair follicle cycles which consist of the growth phase (anagen), regression phase (catagen), and rest phase (telogen) throughout adult life [Id., citing Agabalyan, N A et al. Exp. Dermatol. (2017) 26: 505-509]. During catagen (a short transitional phase that marks the end of active hair growth) and telogen (a resting period where hair growth ceases), follicles prepare their stem cells for the next anagen (the active growth phase of the hair follicle). During anagen, bulge stem cells are activated by induction signals from the dermal papilla and migrate downward to the bulb region, where they proliferate and differentiate to regenerate the inner and outer root sheath, matrix, and hair shaft. Human hair follicle-derived MSCs are dermal papilla or sheath cells from human hair follicles that express the MSC immunophenotype and possess multi-lineage differentiation potential [Id., citing Liu, J Y et al. Tissue Eng. Part A (2010) 16: 2553-2564].

The follicle is surrounded by two sheaths—an inner and outer sheath. These sheaths protect and mold the growing hair shaft. The inner sheath follows the hair shaft and ends below the opening of a sebaceous (oil) gland, which produces sebum, a natural conditioner and sometimes an apocrine (scent) gland. The outer sheath continues all the way up to the gland. An erector pili muscle attaches below the gland to a fibrous layer around the outer sheath. When this muscle contracts, it causes the hair to stand up.

The primary component of the hair fiber is keratin. Keratins are proteins, long chains (polymers) of amino acids. The hair shaft contains three layers of keratin. The inner layer, which is called the medulla, may not be present. The next layer is the cortex, which makes up the majority of the hair shaft. The outer layer is the cuticle, which is formed by tightly packed scales in an overlapping structure similar to roof shingles. Most hair conditioning products attempt to affect the cuticle. Pigment cells are distributed throughout the cortex and medulla giving the hair its characteristic color.

### Eyelashes and Eyebrows

The main function of the eyebrow is to prevent moisture, mostly salty sweat and rain, from flowing into the eye, an organ critical to sight. The typical curved shape of the eyebrow (with a slant on the side) and the direction in which eyebrow hairs are pointed, make sure that moisture has a tendency to flow sideways around the eyes, along the side of the head and along the nose.

Eyebrows also prevent debris such as dandruff and other small objects from falling into the eyes, as well as providing a more sensitive sense for detecting objects being near the eye, like small insects. Eyebrows also have an important facilitative function in communication, strengthening facial expressions such as surprise, confusion, or anger.

The eyelashes consist of curved sensory hairs originating from the eyelid margins. Compared to scalp skin, the skin of the eyelids contains a thinner epidermis and no hypodermis [Nguyen, B. et al. Am. J. Clin. Dermatol. (2023) 24: 55-67, citing Thibaut, S. et al. Br. J. Dermatol. (2010) 162 (2): 304-310]. Eyelashes are rooted approximately 2 mm deep into the dermis and lack the arrector pili muscles associated with most other hair follicles [Id., citing Thibaut, S. et al. Br. J. Dermatol. (2010) 162 (2): 304-310; Aumond, S. and Bitton, E. J. Optom. (2018) 11(4): 211-22]. Humans typically have about 90-160 eyelashes on the lower lids, spread across 5-6 rows, and 75-80 eyelashes on the lower lids, dispersed between 3-4 rows [Id., citing Aumond, S. and Bitton, E. J. Optom. (2018) 11(4): 211-222]. Eyelashes protect the eye from debris and provide a warning that an object (such as an insect or dust mite) is near the eye (which then is closed reflexively).

The inside of the nose contains small hairs called cilia. These cilia and nasal mucus clean the air drawn into the nose of the microscopic particles we inhale, including dust, pollen, and pollutants, for ultimate passage to the lungs.

### Hair Growth

There are three stages of hair growth: anagen, catagen, and telogen.

Anagen is the active growth phase of the hair during which the cells in the root of the hair are dividing rapidly. Anagen hairs are anchored deeply into the subcutaneous fat and cannot be pulled out easily. When a new hair is formed, it pushes the club hair up the follicle and eventually out. During this phase, the hair grows about 1 cm every 28 days. Scalp hair stays in this active phase of growth for 2-6 years. Human subjects that have difficulty growing their hair beyond a certain length have a short active phase of growth. Human subjects that have very long hair have a long active phase of growth. The hair on the arms, legs, eyelashes, and eyebrows have a very short active growth phase of about 30-45 days, which is why they are so much shorter than scalp hair.

The anagen phase is followed by a catagen phase. The catagen phase is a transitional stage that lasts for about 2-3 weeks. About 3% of all hairs are in this phase at any time. During this time growth stops and the outer root sheath shrinks and attaches to the root of the hair. This is the formation of what is known as a club hair.

After catagen, the hair goes into a telogen phase. Telogen is the resting phase, which accounts for 10-15% of all hairs. It lasts for about 100 days for hairs on the scalp and much longer for hairs on the eyebrow, eyelash, arm and leg. During this phase, the hair follicle is completely at rest and the club hair is completely formed. As compared with anagen hair, telogen hair is located higher in the skin and can be pulled out relatively easily. Pulling out a hair in this phase will reveal a solid, hard, dry, white material at the root. Normally, about 25-100 telogen hairs are shed each day.

In the normal scalp, approximately 80 to 90 percent of follicles are growing (anagen), about 5 to 10 percent are resting (telogen), and 1 to 3 percent are undergoing involution (catagen) Each day up to 75 hairs in telogen are shed from the scalp and about the same number of follicles enter anagen.

### Hair Loss

Hair loss typically is categorized as scarring or nonscarring. Those suffering from hair loss often experience embarrassment and the fear being ridiculed by others because they look different. Some may take to wearing oversized eyeglasses in an attempt to hide the absence of eyelashes and/or eyebrows. Loss of nasal cilia may render some more susceptible to respiratory illnesses.

Therapies for hair loss are designed primarily for scalp applications. These include topical minoxidil (Rogain®), antiandrogen agents, including the androgen-receptor blockers sprionolactone, cyproterone acetate, and flutamide, and the 5α-reductase inhibitor finasteride (Propecia®, Merck & Co.). Latanoprost, bimatoprost, and travoprost ophthalmic solution are prostaglandin F2 alpha analogues, marketed for the treatment of ocular hypertension that have the common side effect of eyelash hypertrichosis and trichomegaly. Bimatoprost (0.03%) is the only one approved for hypotrichosis of the eyelashes. However, topical prostaglandins may cause unwanted side effects, such as darkening of the iris color, periocular pigmentation; uveitis, deepening of the superior sulcus and fat atrophy; enhancement of the eyelid crease; and a decrease in proptosis [Starace, M. et al. Dermatol. Ther. (2023) 13: 1243-1253].

### Heat Shock Proteins in Cutaneous Biology

Heat shock proteins are of fundamental importance in cutaneous biology, from protection against UV-induced damage to wound healing and repair. They play important regulatory roles in the control of apoptosis, regulation of steroid aporeceptors, kinases, and other protein remodeling events. They are also implicated in the control of cell growth, and as such, are potential targets for cancer diagnosis and treatment. Currently, emphasis is being placed on the potential use of these proteins in the prevention and treatment of disease.

Cells are repeatedly exposed to environmental or endogenous stresses that can alter normal cell behavior and increase cell vulnerability. In order to ensure tissue integrity and function, cells cope with cellular injuries by adapting their metabolism, protecting essential intracellular constituents, inhibiting cell death signaling pathways and activating those devoted to damage repair.

The molecular chaperones of the heat-shock protein (HSP) family are critical effectors of this adaptive response. They protect intracellular proteins from misfolding or aggregation, inhibit cell death signaling cascades and preserve the intracellular signaling pathways that are essential for cell survival. Most HSPs are rapidly overexpressed in response to cellular injuries including genotoxic stress.

DNA damage can dramatically alter cell behavior and contribute to a number of diseases including developmental defects, neurodegenerative disorders, and cancer. Thus, the ability of cells to repair DNA damage is essential for preserving cell integrity. DNA damage activates a coordinated response that includes detecting DNA lesions before their transmission to daughter cells, blocking cell cycle progression and DNA replication and repairing the damage. Although the role of HSPs in proteins homeostasis and cell death, especially apoptosis, has been widely reported, much less is known about their function in DNA repair.

Hsp72 and hsp27 are among the best investigated stress proteins in skin biology. The hsp70 family consists of two major cytoplasmatic members, namely hsp72 (hsp70), which shows strong stress inducibility and hsp73 (hsc70), which is constitutively expressed in all investigated cells and tissues. Proteins of the hsp70 family have an 18-kD peptide-binding domain at the carboxy-terminal end and a 45-kD ATP-binding domain at the amino-terminal end [Jonak, C. et al. Intl J. Cosmetic Sci. (2006) 28: 233-242, citing Osipiuk, J. et al. Acta Crystallogr. D. Biol. Crystallogr. (1999) 55: 1105-1107; Wu, B. et al. Mol. Cell Biol. (1985) 5: 330-341].

Hsp27 is a member of the ‘small heat shock family’. It can be found in various cells and tissues without prior stress stimulation: breast, uterus, cervix, placenta, platelets, epidermis and adnexal structures [Id., citing Cocca, D R et al. J. Natl Cancer Instit. (1993) 85: 1558-70]. Hsp27 is expressed in different tumor tissues and cell lines. As an intracellular protein, it can be found perinuclearly and is translocated to the nucleus after stimulation. Hsp27 and hsp25 (the murine analogue of hsp27) provide their chaperone function as large oligomer complexes [Id., citing Jakob, U. et al. J. Biol. Chem. (1993) 268: 1517-1520; Rogalla, T. et al. J. Biol. Chem. (1999) 274: 18947-18956].

Phosphorylation of hsp27 leads to the formation of tetramers resulting in a decrease of their protective functions. Since tumor necrosis factor alpha (TNFα) and interleukin 1 (IL-1) can mediate hsp27 activities, it is speculated that this stress protein is involved in the pathogenesis of inflammatory skin diseases [Id, citing Kaur, P et al. FEBS Letters (1989) 227: 175-178; Arrigo, AP. Mol. Cell Biol. (1990) 10: 1276-1280; Guesdon, F. et al. J. Biol. Chem. (1993) 268: 4236-4243]. It has been proven experimentally that hsp27 expression in situ and in vitro correlates with human keratinocyte differentiation [Id, citing Kindas-Muggee, I. and Trautinger, F. Cell Growth Differ. (1994) 5: 777-781; Trautinger, F. et al. Brit. J. Dermatol. (1995) 133: 194-202].

### Epidermal HSPs

Like in all other investigated tissues, the expression of HSPs in human skin is stimulated under stress conditions. In contrast to other cells, keratinocytes show significant basal Hsp72 expression without prior stress [Id, citing Trautinger, F. et al. J. Invest. Dermatol. (1993) 101: 334-338]. Hsp72 is expressed throughout all layers of the epidermis including adnexal structures (hair follicle and sweat gland). Melanocytes, fibroblasts and other (epi)dermal cells are negative for Hsp72 in immunohistochemical staining. Heat shock ex vivo and in vitro results in ‘superinduction’ of Hsp72 in keratinocytes and de novo expression in dermal cells.

The expression of Hsp27 correlates with keratinocyte differentiation and increases continuously from the basal layer to the stratum granulosum [Id., citing Kindas-Mugge, I. and Trautinger, F. Cell Growth Differ. (1994) 5: 777-81; Trautinger, F. et al. Brit. J. Dermatol. (1995) 133: 194-202]. Hsp27 is not detectable immunohistochemically in the stratum corneum. It has been suggested that this finding could be explained either by degradation of hsp27 in corneocytes or by its incorporation into the cornified cell envelope matrix making the antigen inaccessible for antibody staining.

In basal cell carcinomas and squamous cell carcinomas, Hsp27 is absent or sparsely expressed, despite its expression in well-differentiated areas of the tumors [Id., citing Trautinger, F. et al. Brit. J. Dermatol. (1995) 133: 194-202]. Hsp27 overexpression in a transfected squamous carcinoma cell line (A431) results in a delay of tumor development and reduction of tumorigenicity after injection into nude mice [Id., citing Kindas-Mugge, L. et al. Cell Growth Differ. (1996) 7: 1167-1174]. Although Hsp27 has also been described to have a role in thermotolerance, this findings support the hypothesis that in the epidermis Hsp27 is involved mainly in the regulation of cell growth, differentiation and tumorigenicity [Id., citing Welsh, M J and Gaestel, M. Ann. N.Y. Acad. Sci. (1998) 851: 28-35].

### Heat Shock Proteins and UV-Induced Cell Death

Heat-induced inhibition of UVB-induced cell death has been described for in vitro and in situ settings in murine and human skin [Id., citing Mattin, EV., J. Biol. Chem. (1992) 267: 23189-23196; Maytin E V et al. (1993) Cancer Res. (1993) 53: 4952-4959; Maytin, E V et al. J. Invest. Dermatol. (1994) 103: 547-553; Kane, KS, and Maytin, EV. J. Invest. Dermatol. (1995) 104: 62-67; Trautinger, F. et al. J. Invest. Dermatol. (1995) 105: 160-162]. In cell culture using several different assay systems to assess cell viability and function, it has been demonstrated that hyperthermia can reduce damage from UVB. Mild heat conditions have been used and maximal effects were observed when the recovery period between heat and UVB was 6 h. The protective effect disappeared 12 h after heat treatment [Id., citing Trautinger, F. et al. J. Invst. Dermatol. (1995) 105: 160-162].

Investigators have addressed the question of whether hsps are responsible for this effect. Inhibition of protein and mRNA synthesis as well as specific inhibition of Hsp72 block the development of heat-induced UVB tolerance [Id., citing Trautinger, F. et al. J. Invest. Dermatol. (1995) 105: 1600-1662; Simon, M M et al. J. Clin. Invest. (1995) 95: 926-933]. Formation of sunburn cells (SBC), resembling UV-induced apoptotic epidermal keratinocytes, was used as an endpoint in a mouse study [Id., citing Kane, K S and Maytin, E V. J. Invst. Dermatol. (1995) 104: 62-67]. The number of SBC in heat-treated skin (41 °C, 3 h) was significantly reduced compared with a sham control. These results were later also confirmed in human skin [Id., citing Trautinger, F. et al. J. Invest. Dermatol. (1996) 107: 442-443]. Hsp72 is among the gene products responsible for this effect. Indeed, constitutive expression of Hsp72 is an inherent protective mechanism in epidermal cells, and overexpression of Hsp72 is at least in part involved in heat-induced UVB resistance.

Since UV radiation is one of the most abundant and potentially harmful environmental factors for human skin, Roh et al. [Ann. Dermatol. (Seoul) 20(4): 184-189] asked whether sun light exposure induces HSP. The expression of HSP was examined in human epidermal cell lines (normal human keratinocyte (NHK), epidermoid carcinoma A431 cells, normal human melanocytes, and SK30 malignant melanoma cells), in human dermal fibroblasts (HDF), which are the main cellular components of the dermis at baseline, and after heat treatment, UVA irradiation, and UVA+UVB irradiation. HSP70 was detected by immunoblotting using a monoclonal mouse anti-human HSP70 antibody (Santa Cruz Biotechnology, Santa Cruz, CA) detected by peroxidase-conjugated goat anti-mouse immunoglobulins (Tagoimmunologicals®, Camarillo CA). Each of the examined cell types constitutively expressed high amounts of HSP70 without stress conditions. In summary, epidermal cells (NHK, A431 cells, NHMs and SK30 cells) showed high baseline HSP70 expression and no additional upregulation after UV irradiation, whereas HDF showed very low baseline HSP70 expression and high up-regulation of expression after UV irradiation.

### Role of Transient Receptor Potential (TRP) Channels in Skin

TRP channels in skin are crucial for achieving temperature sensitivity to maintain internal temperature balance and thermal homeostasis, as well as to protect skin cells from environmental stresses such as infrared (IR) or near-infrared (NIR) radiation via Hsp production [Hsu, W-L and Yoshioka, T. Biophysics (2015) 11: 25-32].

Many kinds of thermosensitive TRP channels are activated by heat, potentially inducing hsps to respond to a wide variety of physiological and environmental insults. Mammalian TRP channels consist of six-transmembrane cation-permeable channels that are classified into six subfamilies based on amino acid sequence homology: TRPC, TRPV, TRPM, TRPP, TRPA, and TRPML [Id., citing Venkatachalam, K. and Montell, C. Ann. Rev. Biochem. (2007) 76: 387-417]. The functional properties of these six subfamilies of TRP channels are listed and exhibited in Zheng, J. Compr. Physiol. (2013) 3(1): 221-242.

Four heat-activated channels, TRPV1-4, and two cold-activated channels, TRPM8 and TRPA1, belong to temperature-sensitive transient receptor potential channels, and are expressed in dorsal root ganglion sensory neurons, skin, and other cells [Id, citing Clapham, DE. Nature (2003) 426: 517-524]. Temperature sensitivity is crucial to protect against skin damage as well as to maintain internal temperature balance or thermal homeostasis [Id, citing Toth, B I, et al. Br. J. Pharmacol. (2014) 171: 2568-2581]. Thermo TRP channels, TRPV1,3,4, TRPM8, and TRPA1, are known to be expressed in human keratinocytes (KCs); two of them, TRPV1 and TRPA1, are expressed in human skin fibroblasts (HDFs) [Id, citing Venkatachalam, K. and Montell, C. Ann. Rev. Biochem. (2007) 76: 387-417; Toth, B I et al. Br. J. Pharmacol. (2014) 171: 2568-2581], which are major regulators of skin cell proliferation and differentiation, in the skin barrier, and in immune functions responding to skin injury [Id, citing Toth, B I et al. Br. J. Pharmacol. (2015) 171: 2568-2581].

Although the TRPC family does not belong to thermosensitive TRP channels, it controls calcium entry under epidermal receptor stimulation, which is an important part of a functional system for maintaining skin homeostasis [I., citing Beck, B. et al. J. Invest. Dermatol. (2006) 126: 1982-1993].

### TRPV Subfamily

Among the six subfamilies of TRPV channels, TRPV1, TRPV3, and TRPV4 are known to be found in skin cells, especially in keratinocytes (KCs). TRPV1-type channels are activated by heat, low pH, and pro-inflammation [Id., citing Venkatachalam, K. and Montell C. Ann. Rev. Biochem. (2007) 76: 387-417]. It was also reported that TRPV1-type channels are sensitive to some physical stresses such as high (>42 °C) temperature, membrane stretching, and several chemicals (ethanol, lidocaine, mono-acylglycerols, and 2-Aminoethoxydiphenyl borate, 2APB) [Id., citing Vay, L. et al. Br. J. Pharmacol. (2012) 165: 787-801]. Ion selectivity, which is expressed as PCa/PNa, is approximately 10 for chemical stimulation and 4 for physical stimulation [Id, citing Venkatachalam, K. and Montell C. Ann. Rev. Biochem. (2007) 76: 387-417].

TRPV1 is also expressed in dermal fibroblasts [Id, citing Lan, C C et al. J. Dermatol. Sci. (2013) 72: 290-295]. The TRPV1 channel in skin was found to be essential for the control of skin growth as well as for barrier functions, cutaneous immunological functions, skin pathology and several cutaneous diseases [Id, citing Toth, B I et al. Br. J. Pharmacol. (2014) 171: 2568-2581], and skin aging [Id, citing Lan, C C. et al. J. Dermatol. Sci. (2013) 72: 290-295]. TRPV1 can be activated directly by OAG, a membrane-permeable diacylglycerol (DAG) analog, although the 1-oleoyl-2-acetyl-sn-glycerol (OAG)-induced calcium response is one-fifth of the capsaicin-induced signal [Id, citing Woo, D H et al. Mol. Pain (2008) 4: 42]. The activation of TRPV1 by membrane-permeable OAG does not mean it is activated by G protein-coupled receptor (GPCR), because DAG produced by the hydrolysis of phosphatidylinositol 4,5-bisphosphate (PIP2) by phospholipase c (PLC) R is phosphorylated to phosphatidic acid (PA) by DAG kinase immediately [Id, citing Sandal, M. et al. Curr. Protein Pept. Sci. (2013) 14: 650-657]. PIP2 appears to bind to the TRPV1 channel directly, causing channel inhibition that is relieved by PLC β-catalyzed PIP2 hydrolysis. The C-terminal of TRPV1 transcription disrupts the PIP2 binding site and impairs thermal responsiveness.

Moreover, TRPV3 is sensitive to warm temperature (33 °C-39 °C), and PCa/PNa is around 3. Heat-activated TRPV3 current displays strong outward rectification, striking thermal hysteresis, and sensitization following repeated activation [Id, citing Ramsey, I S et al. Ann. Rev. Physiol. (2006) 68: 619-647]. The TRPV3 channel's roles in the skin include nociception, skin integrity, wound healing, hair growth, and sebocyte functions. Similar to the TRPV3 channel, the TRPV4 channel covers a temperature range (27 °C-34 °C), and the ratio of PCa/PNa is close to 6. Unlike TRPV1-TRPV3, TRPV4 is apparently insensitive to activation by 2APB [Id, citing Chung, M K et al. J. Neurosci. (2004) 24: 5177-5182; Mergler, S. et al. J. Cell Physiol. (2011) 226: 1828-2842]. The TRPV4 channel is involved in mechano-sensation, osmo-sensation, nociception, modulation of cell migration, the shear stress sensor, and control adherence junction in the skin cochlea [Id, citing Nilius, B. and Owsianik, G. Genome Biol. (2011) 12: 218].

### TRPA1

The physiological functions of TRPA1 are summarized as follows: thermo-sensation, the most versatile chemo-sensor, mechanical sensation, nociception and, olfactory transduction [Id, citing Nilius, B. and Owsianik, G. Genome Biol. (2011) 12: 218].

TRPA1, well known as a noxious cold pain sensor, is also expressed highly in KCs [Id, citing Donnerer, J. et al. Pharmacology (2012) 89: 7-12] and in HDFs [Id., citing Nilius, B. and Owsianik, G. Genome Biol. (2011) 12: 218]. The role of this channel may be essentially different from those of other TRPV channels because it is directly activated by intracellular Ca2+ through binding to the N-terminal EF-hand domain of TRPA1 [Id, citing Zayats, V. et al. J. Mol. Model. (2013) 19: 4689-4700]. The mechanism underlying this action was suggested to be activation and/or sensitization of TRPA1 channels by GPCR coupled to PLC β signaling, such as bradykinin receptors [Id, citing Wilson, S R et al. Nat. Neurosci. (2011) 14: 595-602].

In addition, since TRPA1 is involved in inflammatory pain (increased sensitivity to painful stimuli), TRPA1 may underlie some components of inflammatory hyperalgesia (increased sensory neurons for cold hyperalgesia). Thus, TRPA1 is most likely a chemical sensor for injury and inflammation [Id, citing Obata, K. et al. J. Clin. Invest. (2005) 115: 2393-2401]. Many TRPA1 ligands, such as mustard oil, acrolein, formalin, and iodoacetamide, can activate TRPA1 extracellularly, while acetaldehyde, H₂O₂, 15d-prostaglandin J2, and prostaglandin A2 (PGA2), all of which are called reactive electrophilic species (RES), work intracellularly [Id, citing Bang, S. and Hwang, SW. J. Gen. Physiol. (2009) 133: 257-62]. These RESs are generated under oxidative stress with the chemical reactivity being transferred from reactive oxygen species (ROS) to RES [Id, citing Bang, S. and Hwang, S W. J. Gen. Physiol. (2009) 133: 257-262]. It is likely that these ROS promote disulfide formation between vicinal thiol residues, resulting in TRPA1 dysfunction [Id., citing Bang, S. and Hwang, SW. J. Gen. Physiol. (2009) 133: 257-262]. However, TRPA1 activation is recovered by the application of dithiothreitol (DTT), which reduces the disulfide bond to the —SH group of cysteine in TRPA1 [Bang, D. et al. Mol. Biosyst. (2009) 5: 750-756].

The sensitivity of TRPA1 to intracellular Ca²⁺ and ROS accelerates cell depolarization, which will activate voltage-dependent Ca²⁺ channels, leading to skin aging, carcinogenesis, and apoptosis [Id, citing Nagata, K. et al. J. Neurosi. (2005) 25: 4052-4061]. Thus, TRPA1 may help regulate the proliferation and differentiation of KCs and skin inflammation [Id, citing Andrade, E L et al. Pharmacol. Ther. (2012) 133: 189-204]. Some functional properties of TRPA1 depend on its ability to interact with TRPV1. The coexistence of TRPA1 with TRPV1 seems to be important for the action of an endogenous mediator, such as bradykinin or calcium [Id, citing Bessac, B F and Jordt, SE. Physiology (Bethesday) 2008 23: 360-370]. Akopian et al. also found that TRPV1 can regulate the mediator of TRPA1 by Ca²⁺ through the PIP2 signaling pathway [Id, citing Akopian, A N et al. J. Physiol. (2007) 583: 175-193].

### TRPM8

The TRPM channel controls certain skin functions, especially those related to melanocyte biology. TRPM8 displays a cold receptor of the body, and its activation can be modulated by many cooling compounds and many other odorant agents isolated from plants, e.g., linalool, geraniol, and hydroxycitronellal [Id, citing Behrendt, H J et al. Br. J. Pharmcol. (2004) 141: 737-745; Harteneck, C. Naunyn Schmiedebergs Arch. Pharmacol. (2005) 371: 307-314]; and the selectivity of Ca²⁺ is about three times higher than that of Na⁺ [Id, citing Venkatachalam, K. and Montell C. Ann. Rev. Biochem. (2007) 76: 387-417].

These results were confirmed electrophysiologically using whole-cell patch clamp experiments [Id, citing Voets, T. et al. Nature (2004) 430: 748-753]. It was also confirmed that the TRPM8 channel was activated by depolarization, which means that the TRPM8 channel is very sensitive to changes in the cell's ionic balance. Thus, it is natural to consider that the TRPM8 channel is involved in body temperature regulation [Id, citing Gavva, N R et al. Mol. Pain (2012) 8: 36].

### TRPC6 and TRPC7

The TRPC subfamily was established as the first recognized mammalian TRP channel [Id, citing Hardie, RC. J. Physiol. (2007) 578: 9-24]. According to the results obtained by Sakura et al., the TRPC subfamily is divided into three groups on the basis of sequence alignments: TRPC1/4/5, TRPC3/6/7, and TRPC2 [Id, citing Sakura, H. and Ashcroft, F M. Diaetologia (1997) 40: 528-532]. TRPC1/4/5/7 channels are expressed in HaCaT, an immortal human keratinocyte line [Id, citing Beck, B. et al. Cell Calcium (2008) 43: 492-505], while Cai et al. detected TRPC1/C5/C6/C7 in gingival KCs [Id., citing Cai, S. et al. J. Dermatol. Sci. (2005) 40: 21-28]. TRPC7 is also a receptor-operated, DAG-activated channel in KCs, mediating a PLC β4-activated transducer current in intrinsic photosensitivity [Id, citing Beck, B. et al. J. Invest. Dermatol. (2006) 126: 1982-1993; Hardie, RC. Handb. Exp. Pharmacol. (2014) 223: 795-826]. As was previously described, most of the TRPC subfamily shows similar characteristics for activation by DAG and inhibition by SKF96365. Thus, it is reasonable to discuss the characteristics of TRPC6 and TRPC7 as representative of the TRPC subfamily in KCs, since these channels are activated by membrane-permeable DAG [Id, citing Beck, B. et al. Cell Calcium (2008) 43: 492-505].

Muller et al. suggested that TRPC6 is a specific channel for inducing KCs differentiation [Id, citing Muller, M. et al. J. Biol. Chem. (2008) 283: 33942-33954]. Eventually, several members of the canonical TRPC subfamily were identified in the skin, where they mostly control the growth and differentiation of KCs under physiological and pathological conditions [Id, citing Toth, B I et al. Br. J. Pharmacol. (2014) 171: 2568-2581. These TRPC channels in the skin are considered to be significantly involved in skin cell growth, barrier function, and cutaneous diseases. Based on the above discussion, as TRP channels open through oxidative stress, intracellular calcium elevation induces ROS and ATP production in mitochondria resulting in disulfide bonds formation to protein dysfunction.

### The Skin as an Immune Organ

The skin harbors a highly specialized immunological niche crucial for the maintenance of tissue homeostasis, defense, and repair. It protects the host from invasion by employing physical barriers, biomolecules, and an intricate network of resident immune and non-immune cells and skin structures. In the absence of a challenge, resident immune cells promote skin physiological functions.

### Physical Barriers

The barrier function of the epidermis is mainly mediated by corneocytes in the stratum corneum. These cells are organized in a “bricks and mortar” manner, interspersed by lipids such as ceramides, cholesterol, and free fatty acids [Nguyen, A V and Soulika, A M, Intl J. Molec. Sci. (2019) 20 (8): 1811, citing Mnon, G K et al. Int. J. pharm. (2012) 435: 3-9]. Each corneocyte contains a lipid envelope linked to keratin filament bundles that fill the intracellular compartments of the corneocyte, thus strengthening its rigidity [Id., citing Madison, KC. J. Investig. Dermatol. (2003) 121: 231-241]. The stratum corneum is composed of three layers and it is both an outside—in barrier to prevent the entry of foreign substances and microorganisms, and an inside-out barrier to prevent water loss [Id, citing Kubo, A. et al. Sci. Rep. (2013) 3: 1731].

The formation of the physical barrier of the skin function depends on junction adhesion molecules and tight junction proteins. Junctional adhesion molecules (JAMs), claudins, zonula occludens-1 (ZO-1), and occludins are found in epidermal layers.

### Biomolecules of the Skin

The main classes of biomolecules that participate in skin defense by disrupting bacterial membranes are antimicrobial peptides (AMPs) and lipids [Id, citing Niyonsaba, F. et al. Exp. Dermatol. (2017) 26: 989-998; Niyonsaba, F. et al. Crit. Rev. Immunol. (2006) 26: 545-576; Van Smeden, J. and Bouwstra, J A. Curr. Probl. Dermatol. (206) 49: 8-26]

AMPs are amphipathic peptides that are expressed constitutively or induced after cell activation in response to inflammatory or homeostatic stimulation. The most thoroughly studied AMP families in human skin are the defensins and the cathelicidins, which are produced by a variety of cells in the skin such as keratinocytes, fibroblasts, dendritic cells, monocytes, and macrophages, and sweat and sebaceous glands [Id, citing Niyonsaba, F. et al. Exp. Dermatol. (2017) 26: 989-998; Niyonsaba, F. et al. Crit. Rev. Immunol. (2006) 26: 545-576]. AMPs are produced as propeptides and become active after proteolytic cleavage. In contrast to defensin family members, which are produced by distinct genes, there is only one gene associated with cathelicidins (cathelicidin antimicrobial peptide—CAMP) [Id, citing Pfalzgraff, A. et al. Front. Pharmacol. (2018) 9: 281]. A number of active peptides can be generated via proteolytic cleavage of the inactive CAMP product, but the most studied cathelicidin is LL-37 [Id, citing Ann. Dermatol. (2012) 24: 126-135]. AMPs may act synergistically and have broad activity against microbial species [Id., citing Clausen, M L and Agner, T. Curr. Probl. Dermatol. (2016) 49: 38-46; Hanson, M A et al. eLife (2018) 493817].

AMPs have roles in modulating host immune responses. Human LL-37 was shown to induce differentiation of monocyte-derived dendritic cells, subsequent cytokine production, and expression of the co-stimulatory molecule CD86 [Id, citing Davidson, D J et al. J. Immunol. (2004) 172: 1146-1156]. LL-37 and β-defensins can also serve as alarmins for keratinocytes by inducing their proliferation and migration [Id, citing Tokumaru, S. et al. J. Immunol. (2005) 175: 4662-4668]. Furthermore, human LL-37 exerts its alarmin effects on immune cells in a synergistic manner with other inflammatory mediators, such as IL-1β [Id, citing Yu, J. et al. J. Immunol. (2007) 179: 7684-7691]. Human α- and β-defensins serve as chemoattractants for activated neutrophils, memory and naïve T cells and immature dendritic cells [Id, citing Zhang, L-J and Gallo, R L. Curr. Biol. (2016) 26: R14-R19], while β-defensins 3 and 4 can recruit monocytes and macrophages [Id, citing Oppenheim, J J and Yang, D J C. Curr. Opin. Immunol. (2005) 17: 359-365].

In addition, AMP functions have been associated with the processes of aging and memory [Id., citing Reinholz, M. et al. An. Dermatol. (2012) 24: 126-135; Zhang, L-J. and Gallo, R L. Curr. Biol. (2016) 26: R14-R19; Lezi, E. et al. Neuron (2018) 97: 125-138]. LL-37 has been shown to exert proangiogenic effects and may also play a role in tissue repair [Id, citing Koczulla, R. et al. J. Clin. Invest. (2003) 111: 1665-1672].

Lipids such as sphingomyelin, glucosylceramides, and phospholipids are intermediate molecules, readily converted into sphingosine and dihydrosphingosine, which exert antimicrobial activity against certain bacterial strains such as Staphylococcus aureus, Streptococcus pyogenes, Micrococcus leutus, and Proprionibacterium acnes [Id, citing Bibel, D J et al. J. Investig. Dermatol. (1992) 98: 269-273]. These lipids are stored in lamellar bodies found in corneocytes in the stratum corneum [Id, citing Van Smeden, J. Biochim. Biophys. Acta (2014) 1841: 295-313].

Sebocytes residing in the sebaceous glands produce sebum, which is rich in lipids such as triacylglycerol, wax esters, non-esterized fatty acids, and squalene. There is a consensus that sebum serves as a “seal” for the hair follicles, thus preventing entry of microbes into the deeper layers of the skin. The AMP dermcidin is expressed by sebocytes, suggesting that sebum exerts defensive functions [Id, citing Dahlhoff, M. et al. J. Dermatol. Sci. (2016) 81: 124-126]. Furthermore, sebum can be further processed into free fatty acids by skin commensal bacteria [Id, citing Kendall, A C et al. Prog. Lipid Res. (2013) 52: 141-164; Bibel, D J et al. J. Investig. Dermatol. (1989) 92: 632-638], and in humans, sebum-derived free fatty acids induce β-defensin 2 expression by sebocytes, further suggesting that sebaceous glands serve an innate defensive role [Id, citing Nakatsuji, T. et al. J. Investig. Dermatol. (2010) 130: 985-994].

The pH of human skin is 5.4-5.9, which makes the skin an inhospitable environment for potential pathogens [Id, citing Schmid-Wendtner, M H and Korting, HC. Skin Pharmacol. Physiol. (2006) 19: 296-302; Fluhr, J W et al. J. Investig. Dermatol. (2001) 117: 44-51]. Furthermore, the dramatic difference in pH levels between the skin (pH 5.4-5.9) and the blood (pH=7.4) serves as a secondary defensive mechanism in the event that microbes breach the skin tissue and enter the circulation.

There are various ways that the skin maintains a low pH. Filaggrin, a filament-associated protein that binds keratin fibers, is broken down into histidine, which is further processed by histidase, expressed by corneocytes into the acidic metabolite trans-urocanic acid [Id, citing Scott, IR. Biochem. J. (1981) 194: 829-838]; this has been implicated in the acidification of the stratum corneum [Id, citing Krien, P M and Kermici, M. J. Investig. Dermatol. (2000) 115: 414-420]. Fatty acids produced in the stratum corneum also alter the acidity of the skin [Id, citing Bibel, D J et al. J. Investig. Dermatol. (1989) 92: 632-638; Fluhr, J W et al. J. Investig. Dermatol. (2001) 117: 44-51]. In addition, sweat glands produce acidic electrolytes and lactic acid, which lowers the pH of the skin [Id, citing Wilke, K. et al. Int. J. Cosmet. Sci. (2007) 29: 169-179] and promotes epidermal turnover [Id, citing Thueson, D O et al. Dermatol. Surg. (1998) 24: 641-645].

The physiological pH of the skin is hospitable for commensal bacteria such as Staphylococcus epidermidis, preventing pathogenic strains such as Staphylococcus aureus from establishing infections in the host [Id, citing Korting, H C et al. Acta Derm. Venereol. (1990) 70: 429-431; Elias, P M. Semin. Immunopathol. (2007) 29: 3-14].

### Immune Cells of the Skin

Skin-resident immune cells promote tissue function in homeostasis and act as sentinels by actively sampling environmental antigens. Both myeloid and lymphoid cell subsets are found in the skin in steady state. Some of these resident immune cells migrate to lymph nodes to either induce peripheral tolerance to tissue self-antigens or initiate robust immune responses. In the event of a challenge, such as infections or tissue injury, immune cells resident in the skin and those infiltrating from the periphery interact to create an intricate defense network to resolve the insult and restore the tissue to its original state.

### Myeloid Cells

Skin-resident myeloid cells include Langerhans cells, dermal dendritic cells, macrophages, mast cells, and eosinophils. Neutrophils are rarely found in healthy skin and thus are not “skin-resident cells.” However, neutrophils populate the skin in inflammatory conditions and after a wound.

Skin-resident myeloid cells contribute to skin homeostasis by secreting growth factors needed for the survival of keratinocytes, fibroblasts, and endothelial cells. In addition, they maintain optimal tissue function by phagocytosing debris and apoptotic cells, supporting vasculature integrity, and promoting tolerance.

In inflammatory conditions, myeloid cells respond immediately and produce pro-inflammatory mediators that drive the activation of cells in the local vicinity and infiltration of the affected site by peripheral immune cells. Skin myeloid cells also serve as a liaison between the innate and adaptive immune system.

### Langerhans Cells

Langerhans cells (LCs) are the sole myeloid cell type in the epidermis. Phenotypically, LCs are characterized by high expression of MHC class II and the presence of langerin+ Birbeck granules [Id, citing Deckers, J. et al. Front. Immunol. (2018) 9: 93; Kaplan, DH. Nat. Immunol. (2017) 18: 1068-1075]. LC maintenance depends on keratinocyte-derived IL-34 [Id, citing Kaplan, DH. Nat. Immunol. (2017) 18: 1068-1075; Ginhoux, F. et al. Science (2010) 330: 841-5; Greter, M. et al. Immunity (2012) 37: 1050-1060; Wang, Y. et al. Nat. Immunol. (2012) 13: 753-760], one of the ligands for the colony stimulating factor-1 receptor (CSF-1R). CSF-1R is constitutively expressed on LCs and global deletion of this receptor results in a total lack of LCs [Id, citing Wang, Y. et al. Nat. Immunol. (2012) 13: 753-760; Wang, Y. and Colonna, M. Eur. J. Immunol. (2014) 44: 1575-1581; Ginhoux, F. et al. Nat. Immunol. (2006) 7: 265-273].

LCs are derived from two sources, the extra-embryonic yolk sac and fetal liver monocytes. LCs renew from a local progenitor cell [Id, citing Merad, M. et al. Nat. Immunol. (2002) 3: 1135-1141]. In homeostasis, LCs anchor themselves within the epidermis through interactions between epithelial cell adhesion molecules (EpCAM) or E-cadherin expressed on LCs and E-cadherin expressed by keratinocytes [Id., citing Deckers, J. et al. Front. Immunol. (2018) 9: 93; Choi, H W et al. Clin. Exp. Dermatol. (2018) 43: 291-295; Mayumi, N. et al. Eur. J. Immunol. (2013) 43: 270-280].

Furthermore, autocrine and paracrine TGFβ signaling restricts LCs in the epidermis by regulating the expression of E-cadherin on LCs [Id., citing Bobr, A. et al. Proc. Natl Acad. Sci. USA (2012) 109: 10492-10497; Kel, J M et al. J. Immunol. (2010) 185: 3248-3255] and increases phagocytic behavior in LCs during steady state [Id., citing Bauer, T. et al. J. Exp. Med. (2012) 209: 2033-2047]. While anchored, LCs sample antigens and, upon activation, they can extend their processes from the cell body outward to the stratum corneum or below toward the stratum basale [Id., citing Deckers, J. et al. Front. Immunol. (2018) 9: 93]. LCs participate in tight junction formation and thus can sample the microenvironment without damaging the barrier [Id., citing Decker, J. et al. Front. Immunol. (2018) 9: 93].

LCs are migratory cells and continually travel to the skin draining lymph nodes to promote tolerance in homeostasis [Id., citing Ghigo, C. et al. J. Exp. Med. (2013) 210: 1657-1664] or to initiate adaptive immune responses [Id., citing Romani, N. et al. J. Exp. Med. (1989) 169: 1169-1178; Romani, N. et al. J. Exp. Med. (1989) 93: 600-609; Schuler, G. and Steinman, R M. J. Exp. Med. (1985) 161: 526-546].

The migration of LCs (and also of dermal dendritic cells) through the dermis is mediated via CXCR4 signaling after binding to its cognate chemokine CXCL12, which is produced by dermal fibroblasts [Id., citing Kabashima, K. et al. Am. J. Pathol. (2007) 171: 1249-1257].

In the presence of inflammatory mediators and other activators such as pathogen-associated molecular patterns (PAMPs) and danger-associated molecular patterns (DAMPs), LCs upregulate co-stimulatory molecules and migrate from the epidermis en masse to the draining lymph nodes, where they prime adaptive immune responses in a manner equivalent to that of conventional dendritic cells [Id., citing Deckers, J. et al. Front. Immunol. (2018) 9: 93; Wang, B. et al. Immunology (1996) 88: 284-288; Griffiths, C E et al. Cytokine (2005) 32: 67-70; Kashem, S W et al. Immunity (2015) 42: 356-366; Nakajima, S. et al. J. Allergy Clin. Immunol. (2012) 129: 1048-1055]. TGFβ signaling is disrupted during inflammation, thus also promoting LC migration from the epidermis [Id, citing Bobr, A. et al. Proc. Natl Acad. Sci. USA (2012) 109: 10492-10497].

LCs are professional antigen-presenting cells and activate both CD8+ cytotoxic T lymphocytes and CD4+ helper T lymphocytes. In humans, efficient antigen presentation by LCs is dependent upon caveolin-1, a scaffold protein that serves a multitude of functions including transport of lipids, signal transduction, and membrane trafficking [Id, citing Polak, M E et al. J. Investig. Dermatol. (2014) 134: 695-703; Liu, P. et al. J. Biol. Chem. (2002) 277: 41295-41298]. LCs can also exert immunoregulatory and tolerogenic functions [Id, citing Deckers, J. et al. Front. Immunol. (2018) 9: 93; Flacher, V. et al. EMBO Mol. Med. (2014) 6: 1191-1204; Shklovskaya, E. et al. Proc. Natl Acad. Sci. USA (2011) 108: 18049-18054].

### Dermal Dendritic Cells

Dendritic cells that reside in the dermis are known as dermal dendritic cells (dDCs). In a similar manner to LCs, dDCs migrate to the lymph nodes, and are professional antigen-presenting cells efficient at priming adaptive immune responses [Id, citing Hain, T. et al. J. Investig. Dermatol. (2018) doi: 10.1016/j.jid/2018.08.022; Tomura, M. et al. Sci. Rep. (2014) 4: 6030]. dDCs can induce tolerance to topically applied antigens encountered in the hair follicles [Id, citing Tordesillas, L. et al. Nat. Commun. (2018) 9: 5238].

Unlike LCs, all dDCs are derived from progenitors originating from the bone marrow, with replenishment occurring roughly every seven days [Id, citing Kim, T G et al. Intl J. Mol. Sci. (2017) 19: 42; Ober-Blobaum, J L, et al. Methods Mol Biol. (2017) 1559: 37-52]. The two main subsets of dDCs are the conventional dendritic cell type 1 (cDC1) and the conventional dendritic cell type 2 (cDC2) [Id., citing Ober-Blobaum, J L, et al. Methods Mol Biol. (2017) 1559: 37-52; Kashem, S W et al. Annu. Rev. Immunol. (2017) 35: 466-499].

dDCs are implicated in maintaining homeostatic interactions between the host and skin-resident commensal bacteria. Plasmacytoid DCs (pDCs) are a DC subset, found in the skin exclusively under inflammatory conditions. pDCs are mass producers of IFNα, which is essential for viral defense [Id., citing Kashem, S W et al. Annu. Rev. Immunol. (2017) 35: 466-993].

Human dDCs are subdivided into cDC1 (CD141+), cDC2 (CD1c+), and CD14+ dDCs [123]. cDC1s co-express CD304 (neuropilin-1), XCR1, and CD370 (CLEC9A). Unlike murine dDCs, human dDCs do not express langerin [Id., citing Kashem, S W et al. Annu. Rev. Immunol. (2017) 35: 466-499; Haniffa, M. et al. Immunity (2012) 37: 60-73]. Human cDC1s can cross-present in a similar manner to murine cDC1s [Jongbloed, S L et al. J. Exp. Med. (2010) 207: 1247-1260] and are potent in inducing Th1 responses [Id., citing Segura, E. et al. J. Exp. Med. (2012) 209: 653-660]. cDC2s and CD14+ dDCs co-express CD11b and CX3CR1. Both human cDC1s and cDC2s are capable of inducing Th2 responses [Id, citing Segura, E. et al. J. Exp. Med. (2012) 209: 653-660]. Human pDCs express CD304, CD303 and CD123 and like their murine counterparts, they are only found in inflamed skin. Human peripheral blood pDCs upregulate the expression of B cell maturation antigens upon activation via TLR9 signaling, suggesting that pDCs play a role in the maintenance of plasma cells, in addition to their inflammatory functions [Id, citing Schuh, E. et al. J. Immunol. (2017) 198: 3081-3088].

### Macrophages

Macrophages are found in the dermal layer of the skin and require IL-34 for survival [Id, citing Wang, Y. et al. Eur. J. Immunol. (2014) 44: 1575-1581; Ginhoux, F. and Jung, S. Nat. Rev. Immunol. (2014) 14: 392-404]. Two sources of dermal macrophages have been identified so far. The first source is embryo-derived progenitors that seed the skin prenatally and are self-renewing in a similar fashion to LCs [Id, citing Guilliams, M. et al. Nat. Rev. Immunol. (2014) 14: 571-578]. The second and major source of dermal macrophages is circulating monocytes (monocyte-derived macrophages) that mature once they reach the skin. This population replenishes roughly every 10 days [Id, citing Sheng, J. et al. Immunity (2015) 43: 382-393; Baranska, A. et al. J. Exp. Med. (2018) 215: 1115-33].

Monocytes that give rise to dermal macrophages express lymphocyte antigen 6C (Ly6C), and home to the skin in a CCR2-dependent manner [Id., citing Malissen, B. et al. Nat. Rev. Immunol. (2014) 14: 417-428]. As monocytes mature into skin-resident macrophages, the expression of CCR2 (a member of the beta chemokine receptor family, specifically one of the CC chemokine receptors) is downregulated [Id, citing Tamoutounour, S. et al. Immunity (2013) 39: 925-938]. CD64 expression is prominent on dermal macrophages and is used as a marker to differentiate them from the dDCs [Id, citing Baranska, A. et al. J. Exp. Med. (2018) 215: 1115-1133; Tamoutounour, S. et al. Immunity (2013): 39: 925-938]. CD36, DC-SIGN, and IL-10 are highly expressed by macrophages isolated from healthy skin, suggesting that they adapt an immunoregulatory phenotype [Id, citing Tamoutounour, S. et al. Immunity (2013): 39: 925-938; Lonati, A. et al. J. Investig. Dermatol. (1996) 106: 96-101]. In steady state, macrophages remove cellular debris [Id, citing Malissen, B. et al. Nat. Rev. Immunol. (2014) 14: 417-428, Tamoutounour, S. et al. Immunity (2013): 39: 925-938] and have also been implicated in homeostatic hair regeneration [Id., citing Yanez, D A et al. Pflugers Arch. (2017) 469: 455-463; Eichmullelr, S. et al. J. Histochem. Cytochem. (1998) 46: 361-370].

Macrophages are plastic, and one way to categorize their effector functions is as pro-inflammatory “M1” or anti-inflammatory/pro-repair “M2.” M1 macrophages express inducible nitric oxide synthase (iNOS), and secrete inflammatory cytokines such as TNFα, IL-13, and IL-6 [Id., citing Mills, CD. Front. Immunol. (2015) 6: 212; Mills, C D and Ley, K. J. Innate Immun. (2014) 6: 716-726]. M2, or “alternatively activated” macrophages, adopt an anti-inflammatory and/or pro-repair phenotype. M2 macrophages can be further subdivided into M2a, M2b, M2c, and M2d. M2a macrophages are known to be pro-fibrotic due to TGFβ production [Id., citing Ohji, M. et al. Curr. Eye Res. (1993) 12: 703-709]. M2b macrophages express the co-stimulatory molecule CD86 and as such they are the most pro-inflammatory of the M2 subsets.

M2c macrophages are induced by IL-10 or TGFβ, express the Mer tyrosine kinase (MerTK), promote neovascularization [Id., citing Martinez, F O et al. Front. Biosci. (2008) 13: 453-461] and have high scavenging and debris clearing activity. M2d macrophages are responsive to IL-6 and exhibit a few of the properties of tumor-associated macrophages such as secretion of IL-12 and IL-10 [Id., citing Martinez, F O et al. Front. Biosci. (2008) 13: 453-461, Ferrante, C J and Leibovich, S J. Ad. Wound Care (New Rochelle) (2012) 1: 10-16; Arora, S. et al. Immunobiology (2018) 223: 383-396; Wang, Q. et al. Cell Res. (2010) 20: 701-712]. M2d cells are also known to express high levels of the adenosine receptor A2AR in the presence of lipopolysaccharide (LPS) [Id., citing Ferrante, C J et al. Inflammation (2013) 36: 921-931]. A2AR signaling has been shown to attenuate pro-inflammatory cytokine production [Id., citing Koroskenyi, K. et al. Biochim. Biophys. Acta (2016) 1863: 1461-1471], suggesting that A2AR-expressing M2d cells may be involved in the resolution of the inflammatory response.

### Mast Cells

Mast cells are commonly found in the dermal layer. In humans, mast cells are found in all areas of the skin but are most numerous in the arms and the legs [Id., citing Janssens, A S et al. J. Clin. Pathol. (2005) 58: 285-289]. The density of mast cells in the papillary dermis increases with age; they are most often localized in the proximity of PGP9.5+ nerve fibers expressing vasoactive intestinal peptide (VIP), which was shown to suppress mast cell degranulation [Id., citing Pilkington, S M et al. Br. J. Dermatol. (2018) doi: 10.111.bjd.17268]. This has been associated with the reduction of the amount of extracellular matrix remodeling in the skin observed during the later stages of life.

Mast cells contain granules containing preformed mediators such as histamine, sulfated proteoglycans, serotonin, and tryptase and/or chymase. In both humans and mice, mast cells resident in the skin express both tryptase and chymase, whereas other tissue-resident mast cells express only tryptase [Id., citing Olivera, A. et al. J. Allergy Cli. Immunol. (2018) 142: 381-383].

Mast cells are classically known for their involvement in allergic reactions as they produce and release copious amounts of histamine when their Fcε receptors are crosslinked via IgE-antigen complexes [Id, citing Shi, L B et al. Pestic. Biochem. Physiol. (2018) 148: 159-165; Hirano, T. et al. Sci. Rep. (2018) 8: 14237]. They also make large amounts of prostaglandin D2 (PGD2), a lipid-derived inflammatory mediator.

Mast cells are mass producers of leukotrienes (LTs), which are short-lived lipid inflammatory mediators synthesized via the 5-lipoxygenase (5-LO) pathway.

A variety of cytokines and growth factors are produced by mast cells either constitutively or in response to a stimulus [Id, citing Mukai, K. et al. Immunol. Rev. (2018) 282: 121-150]. Many of these cytokines and growth factors such as TNFα and vascular endothelial growth factor (VEGF) may be pre-formed and packaged in mature mast cell granules [Id, citing Wernersson, S. and Pejler, G. Nat. Rev. Immunol. (2014) 14: 478-494; Gordon, JR, Galli, S J J N. Nature (1990) 346: 274; Grutzkau, A. et al. Mol. Biol. Cell (1998) 9: 875-884]. Proper formation of mast cell granules is mediated mostly by proteoglycan serglycin [Id, citing Braga, T. et al. Biochem. J. (2007) 403: 49-57].

Dermal mast cells can prime adaptive responses during cutaneous infections. Mast cell-derived IL-1β induces production of histamine and IL-8 in human mast cells, suggesting that IL-1β is part of a positive feedback loop for mast cell activation [Id, citing Subramanian, N. and Bray, MA. J. Immunol. (1987) 138: 271-275; Kim, G Y et al. J. Immunol. (2010) 184: 3946-3954]. TNFα produced by mast cells is known to drive migration of dermal dendritic cells to draining lymph nodes in a murine model of hapten-induced contact hypersensitivity [Id, citing Suto, H. et al. J. Immunol. (2006) 176: 4102-4112]. Mast cell-derived TNFα is also crucial for maintenance of tolerance toward allogeneic skin grafts in mice [Id, citing De Vries, V C et al. Immunity (2011) 35: 550-561]. Studies revealed that mast cells are able to form immunological synapses with γδ T lymphocytes when challenged with dengue virus [Id, citing Mantri, CK and St. John, A L. J. Clin. Investig. (2018) doi: 10.1172/JCI122520197] and can acquire MHC class II expression via vesicle transfer from dendritic cells after administration with dinitrofluorobenzene [Id, citing Dudeck, J. et al. J. Exp. Med. (2017) 214: 3791-3811].

### Eosinophils

Eosinophils are skin-resident cells [Id, citing Yu, Y R et al. PLoS ONE (2016) 11: e0150606; Ramirez, G A et al. Biomed. Res. Int. (2018) 99095275], but not much is known about their role in tissue homeostasis. Eosinophilic granules are loaded with potent and toxic proteins: major basic protein (MBP), eosinophil peroxidase (EPO), eosinophil protein X/eosinophil-derived neurotoxin (EPX/EDN), and eosinophil cationic protein (ECP) [Id, citing Long, H. et al. Clin. Rev. Allergy Immunol. (2016) 50: 189-213].

Like mast cells, eosinophils produce inflammatory lipid mediator leukotrienes (LTs) and prostaglandin D2 (PGD2) [Id, citing Luna-Gomes, T. et al. J. Immunol. (2011) 187: 6518-6526], with the latter being crucial for eosinophilic infiltration of the skin in hypersensitivity reactions such as atopic dermatitis [Id, citing He, R. et al. J. Allergy Clin. Immunol. (2010) 126: 784-790]. In addition, eosinophils generate extracellular DNA traps (ETs) containing eosinophil granules [Id, citing Ueki, S. et al. Blood (2013) 121: 2074-2083; Yousefi, S. et al. Nat. Med. (2008) 14: 949-953]. These traps are believed to play a role in antibacterial defense.

Eosinophils are classically known to promote host defense against parasitic infections [Id, citing Jacobesen, E A et al. Blood (2012) 120: 3882-3890]. The roles of eosinophils in dermatoses, or skin diseases associated with eosinophilia such as allergic contact dermatitis and urticaria, is emerging [Id, citing Long, H. et al. Clin. Rev. Allergy Immunol. (2016) 50: 189-213; Foster, E L et al. PLoS ONE (2011) 6: e22029; Oyoshi, M K et al. Proc. Natl Acad. Sci. USA (2012) 109: 4992-7; Wang, S F et al. Med. Inflamm. (2016) 2016: 5032051]. In eosinophilic dermatoses, extensive eosinophilic degranulation in the skin results in local tissue damage.

### Lymphoid Immune Cells

The skin harbors different types of lymphoid cells, all of which are important in both steady state and inflammatory responses. Both human and murine skin contain γδ T lymphocytes and αβ T lymphocytes, along with natural killer T cells. γδ T cells are the dominant T cell population in murine skin, while αβ T cells are the dominant T cell population in human skin [Id., citing Mestas, J. and Hughes, CC. J. Immunol. (2004) 172: 2731-8; Elbe, A. et al. Semin. Immunol. (1996) 8: 341-349].

### αβ T Lymphocytes

In both mice and humans, αβ T lymphocytes are found in the epidermis and dermis [Id., citing 224], and traffic to the skin from the periphery via cutaneous lymphocyte antigen (CLA) interactions with E-selectin (expressed on endothelial cells), which is upregulated under inflammatory conditions [Id., citing Kantele, A. et al. J. Immunol. (1999) 162: 5173-5177; Groves, R W et al. Br. J. Dermatol. (1991) 124: 117-123]. αβ T lymphocytes in the skin are resident memory T cells (T_{RM}), which are long-lived and distinct from their circulating counterparts [Id., citing Seidel, J A et al. Clin. Exp. Immunol. (2018) 194: 79-92].

Most T_{RM} in the skin are derived from antigen-specific effector T cells, which previously infiltrated the tissue as a result of an infection. After resolution, these T_{RM} cells seed all areas of the skin but are denser in areas of antecedent infection [Id., citing Clark, RA. Sci. Trans. Med. (2015) 7: 269rv261]. Following a skin infection, T_{RM} cells are also found in distal organs such as the lung and gastrointestinal tract [Id., citing Clark, RA. Sci. Transl. Med. (2015) 7:269rv261].

T_{RM} express lower levels of CD28 than effector memory T cells, but can mount robust local recall responses [Id., citing Seidel, J A et al. Clin. Exp. Immunol. (2018) 194: 79-92] without emigrating from the tissue to do so [Id., citing Gebhardt, T. et al. Nat. Immunol. (2009) 10: 524-530]. T_{RM} also exert sentinel-like functions by promoting recruitment of other memory T cells from the periphery to sites of infection [Id., citing Ariotti, S. et al. Science (2014) 346: 101-105; Schenkel, J M et al. Nat. Immunol. (2013) 14: 508-513].

The most studied skin T_{RM} are CD8+ T cells. CD8+T_{RM} cells are usually found in the epidermis and may dislocate dendritic epidermal T cells [Id., citing Mackay, L K et al. Nat. Immunol. (2013) 14: 1294-1301]. All CD8+T_{RM} express CD69, an early marker of lymphocyte activation, and a high proportion also express CD103 [Id., citing Mackay, L K et al. Nat. Immunol. (2013) 14: 1294-1301]. CD103 is required for the development of CD8+T_{RM} cells in the skin [Id., citing Mackay, L K et al. Nat. Immunol. (2013) 14: 1294-1301] and mediates adhesion interactions with keratinocytes via an E-cadherin-independent manner [Id., citing Jenkinson, S E et al. Immunology (2011) 132: 188-96].

CD4+ T_{RM} cells also make up a significant portion of the skin-resident lymphocyte population and are found in both the epidermis and dermis [Id, citing Mackay, L K et al. Nat. Immunol. (2013) 14: 1294-1301]. Although the function of CD4+ T cells in the skin is not studied to the same extent as CD8+T_{RM}, they are often the dominant αβ T cell subset. During steady state, clusters of antigen-presenting cells with CD4+ memory T cells are found around the hair follicles in both murine and human skin, and cells in these clusters circulate between the skin and periphery [Id, citing Collins, N. et al. Nat. Commun. (2016) 7: 11514]. These clusters are formed because keratinocytes in the hair follicles produce IL-7 and IL-15, which are required for homeostatic maintenance of the T cell populations [Id, citing Adachi, T. et al. Nat. Med. (2015) 21: 1272-1279].

### γδ T Lymphocytes

Unlike the αβ T lymphocytes, γδ T cells do not undergo the same stringent negative selection process during development and are released from the thymus in waves, with the first wave of γδ T cells seeding the dermis of the skin [Id, citing Sutoh, Y. et al. Front. Immunol. (2018) 9: 1059; Xiong, N. et al. Immunity (2004) 21: 121-131; Xiong, N. and Raulet, DH. Immunol. Rev. (2007) 215: 15-31].

The functions of dermal γδ T cells in inflammatory skin conditions are well documented. This population of dermal T cells is inclined to produce IL-17 [Id., citing Gray, EE. Et al. J. Immunol. (2011) 186: 6091-6095], indicating their significance in cutaneous diseases such as psoriasis [Id, citing Van der Fits, L. et al J. Immunol. (2009) 182: 5836-5845; Mabuchi, T. et al. J. Investig. Dermatol. (2009) 182: 5836-5845].

γδ T cells in the skin can also play a protective role in cutaneous defense; a reported study showed that γδ T cell-deficient mice exhibited larger lesions and reduced IL-17 production in response to Staphylococcus aureus compared to wild type (WT) control mice [Id, citing Cho, J S et al. J. Clin. Investig. (2010) 120: 1762-73].

γδ T cells are not MHC-restricted [Id, citing Champagne, E. Arch. Immunol. Ther. Exp. (2011) 59: 117-37] and can recognize soluble antigens [Id, citing Champagne, E. Arch. Immunol. Ther. Exp. (2011) 59: 117-137; Born, W K and O'Brien, R L. Arch. Immunol. Ther. Exp. (2009) 57: 129-135], antigens derived from damaged or stressed cells [Id, citing O'Brien, R L and Born, W. Semin. Immunol. (1991) 3: 81-87; O'Brien, R I et al. Proc. Nat. Acad. Sci. USA (1992) 89: 4348-4352; Heng, MK. Madame Curie Bioscience Database, Landes Bioscience: Austin TX, SA (2013); pp. 2000-2013], or antigens complexed with non-classical MHC molecules such as CD1b, CD1c, and CD1d [Id, citing Cui, Y. et al. Biol. Direct. (2009) 4: 47; Luoma, A M et al. Trends Immunol (2014) 35: 613-621] or MHCI-related chain A/B (MICA/MICB) [Id, citing Champsaur, M. and Lanier, LL. Immunol. Rev. (2010) 235: 267-285]. One study suggested that γδ T cells also recognize butyrophilin-like Btnl/BTNL proteins, which are part of the B7 superfamily that regulates immune responses via costimulatory or coinhibitory signals [Id, citing Melandri, D. et al. Nat. Immunol. (2018) 19: 1352-1365].

### B Lymphocytes

B cells are rather sparse in the skin in steady state and it is unclear whether they are indeed resident to the skin [Id., citing Nihal, M. et al. J. Mol. Diagn. (2000) 2: 5-20; Geherin, S A et al. J. Immunol. (2012) 188: 6027-6035; Egbuniwe, I U, et al. Trends Immunol. (2015) 36: 102-111]. However, the roles of B lymphocytes in skin inflammatory conditions are well documented. In humans, B cells are found in elevated levels in cutaneous diseases such as atopic eczema, cutaneous leishmaniasis, and cutaneous sclerosis [Id, citing Simon, D. et al. J. Allergy Clin. Immunol. (2008) 121: 122-128; Geiger, B. et al. Br. J. Dermatol. (2010) 162: 870-874; Lafyatis, R. et al. Arthritis Rheum. (2009) 60: 578-583]. B cells are found in the reticular dermis over the course of these diseases and are associated with increased levels of IgM, IgE, and IgG. In a similar manner to T lymphocytes, B lymphocytes traffic to the skin tissue via cutaneous lymphoid antigen (CLA) interactions [Id, citing Postigo, A A et al. J. Clin. Investig. (1994) 94: 1585-1596]. B cells also play a role in delayed-type hypersensitivity reactions in the skin.

### Non-Immune Cells

Pattern recognition receptors (PRR) are expressed by most cells of the skin and have been characterized on keratinocytes, fibroblasts, adipocytes, melanocytes, and endothelial cells [Id, citing Chen, L. and DiPietro, LA. Adv. Wound Care (2017) 6: 344-355]. Activation of these receptors results in the production of cytokines and chemokines by non-immune skin cells, thus participating in the local immune response [Id, citing Ahn, J H et al. Exp. Dermatol. (2008) 17: 412-417; Faure, E. et al. J. Biol. Chem. (2000) 275: 11058-11063; Taylor, K R et al. J. Biol. Chem. (2004) 279: 17079-17084; Song, M J et al. Biochem. Biophys. Res. Commun. (2006) 346: 739-745].

Keratinocyte-derived inflammatory responses have been extensively studied. These cells express almost all intracellular and extracellular PRRs and produce a variety of cytokines, chemokine and AMPs to protect the host against infection [Id., citing Bitschar, K. et al. J. Dermatol. Sci. (2017) 87: 215-220; Pasparakis, M. etal. Nat. Rev. Immunol. (2014) 14: 289; Schittek, B. Curr. Probl. Dermatol. (2011) 41: 54-67]. Keratinocytes both in the epidermis and skin elements are in constant interaction with local immune cells and produce factors crucial in homeostasis and in tissue repair [Id., citing Wang, Y. et al. Nat. Immunol. (2012) 13: 753-760; Adachi, T. et al. Nat. Med. (2015) 21: 1272-1279; Chodaczek, G. et al. Nat. Immunol. (2012) 13: 272-282; Takashima, A. et al. J. Investig. Dermatol. (1995) 105: 505-535]. Other studies demonstrated that keratinocytes produce IL-33 in response to hypo-osmotic stress [Id., citing Pietka, W. et al. J. Invest. Dermatol. (2018 doi 10.1016/j.jld.2018.07.023], and that human and murine keratinocytes produce IL-6 and IL-1β mediated by NFκB signaling in response to UVB irradiation [Id., citing Tang, S C et al. J. Dermatol. Sci. (2017) 86: 238-248].

Fibroblasts immunomodulatory functions have also been well delineated. They express PRRs, synthesize many cytokines, and were shown to produce anti-microbial peptides (AMPs) [Id., citing Bautista-Hernandez, L A et al. Eur. J. Microbiol. Immunol. (2017) 7: 151-157]. Dermal fibroblasts and keratinocytes produce serum amyloid A in response to PRR signaling [Id., citing Morizane, S. et al. Clin. Exp. Dermatol. (2018) doi: 10.111/ced.13604], which is believed to induce the production of pro-inflammatory cytokines from various immune cells [Id., citing Eklund, K K et al. Crit. Rev. Immunol. (2012) 32: 335-348]. A reported investigation showed that human fibroblasts cultured in vitro produced massive amounts of TNFα, IL-1β, IL-6, IL-8, and IL-25 when subjected to thermal stress [Id., citing Jiang, L. et al. Biomed. Pharmacother. (2018) 107: 24-33].

### Influence of Circadian Oscillations in Skin on Immune Response, Skin Homeostasis, Stress Mediation, and Aging

The circadian clock, an intrinsic autonomous clock that controls the body's divergent functions during day and night, affects the expression of multiple genes that mediate skin stem cell metabolism and proliferation, DNA repair, stimulus response and immunity [Duan, J. et al. FEBS Lett (2021) 595 (19): 2413-2436]. Circadian rhythm is closely linked to immunological processes and skin homeostasis, and its dysynchrony can be linked to the perturbation of the skin [Salazar, A. and von Hagen, J. Intl Jm Molec. Sci. (2023) 24: 5635].

The mammalian circadian clock at a cellular level consists of at least 3 overlapping feedback loops (FIG. 3) [Id., citing Sherratt, M J et al. Matrix Bio. (2019) 84: 97-110; Lyons, A B et al. J. Clin. Aesthet. Dermatol. (2019) 12: 42-45; Curtis, A M et al. Immunity (2014) 40: 178-186].

In the first loop, the core circadian clock proteins BMAL1 (basic helix-loop-helix ARNT like 1; also called ARTNL, aryl hydrocarbon receptor nuclear translocator-like) dimerizes either with CLOCK (circadian locomotor output cycles kaput) or with NPAS2 (neuronal PAS domain protein 2) and then triggers the expression of PER (period circadian regulator; PER1-3), CRY (cryptochrome circadian regulator; CRY1-2), ROR (or RAR, related orphan receptor), NR1D1 (nuclear receptor subfamily 1 group D member 1; also called REV-ERE), DBP (D-box binding PAR Bzip transcription factor), and other clock controlled genes by binding to their E-box elements (5′-CACGTG-3′) in the promoter region.

On reaching a critical concentration, the proteins PER and CRY dimerize to inhibit their own expression by preventing the binding of BMAL1: CLOCK to DNA, which results in an oscillation of these proteins [Id., citing Sherratt, M J et al. Matrix Biol. (2019) 84: 97-110; Lyons, A B et al. J. Clin. Aesthet. Dermatol. (2019) 12: 42-45; Curtis, A M et al. Immunity (2014) 40: 178-186; Richards, J. and Gumz, ML. Am. J. Physiol. Regul. Integr. Comp. Physiol. (2013) 304: R1053-R1064].

In the second loop, the protein ROR binds to the RORE element 5′-(A/G)GGTCA-3′ in the promoter of BMAL1, CLOCK, and NFIL3 (nuclear factor, interleukin 3 regulated), resulting in their transcription. The binding of ROR to the RORE element is inhibited by NR1D1, and potentially also by related proteins from this family. This completes the second loop.

The protein DBP, whose expression is under the control of BMAL1:CLOCK from the first loop, binds to the D box (5′-TTATG(T/C)AA-3′) in the promoter region of PER. This binding is negatively regulated by NFIL3 from the second loop. Taken together, this is considered the third loop.

Similar to PER1/2, CRY1 is regulated by a combinatorial mechanism involving both E-box and RORE, giving rise to a phase distinct from DBP and REV-ERB. The DEC loop is ancillary to the core circadian loops and is characterized by the expression of DEC and other circadian controlled genes, which are under the control of BMAL1: CLOCK. DEC, in turn, inhibits the binding of BMAL1: CLOCK to the E-box element, thereby regulating its own expression [Id., citing Ono, D. et al. Sci. Rep. (2021) 11: 19240; Honma, S. et al. Nature (2002) 419: 841-844].

Posttranslational modifications (PTMs) and proteasomal degradation of these core components of the molecular clock machinery are essential to maintain the oscillatory nature of these proteins and the proteins they regulate. These PTMs include phosphorylation, glycosylation, ubiquitination, acetylation, and SUMOylation, as reviewed by Hirano et al. [Id., citing Hirano, A. et al. Nat. Struct. Mol. Biol. (2016) 23: 1053-1060]. In some cases, a further level of complexity is introduced in the form of crosstalk between these mechanisms; for instance, O-linked β-N-acetylglucosamine (O-GlcNAc) competing for the same serine and threonine residues as kinases for phosphorylation [Id., citing Hirano, A. et al. Nat. Struct. Mol. Biol. (2016) 23: 1053-1060; Hardin, P E and Panda, S. Curr. Opin. Neurobiol. (2013) 23: 724-731]

### Zeitgebers and the Circadian Clock in Mammalian Skin

The intracellular molecular clock oscillates in response to environmental signals known as ‘Zeitgebers’, derived from German and directly translating to ‘time giver’. As a result, in the study of circadian rhythm, time in days is often divided into ‘zeitgeber time’. Light is the primary zeitgeber. It is detected via the optical nerve, which then transmits signals of perceived light to the hypothalamic suprachiasmatic nucleus (SCN [Id., citing Curtis, A M et al. Immunity (2014) 40: 178-186]. This information is used to entrain the functional molecular clocks in peripheral tissues via the autonomic nervous system and the hypothalamus pituitary adrenal axis via hormones including glucocorticoids and catecholamines (epinephrine and norepinephrine) [Id., citing Kalsbeek, A. et al. Mol. Cell Endocrinol. (2012) 349: 20-29].

The hormones prolactin, growth hormone, and melatonin have been implicated in circadian signaling. Other zeitgebers that have been exploited in in vivo studies include sleep wake cycles, feeding and fasting regimes, and temperature. These zeitgebers are linked to the presence of light, as well as circulating levels of melatonin [Id., citing Sato, K. et al. J. Pineal. Res. (2020) 688: e12639; Slominski, A T et al. J. Investig. Dermatol. (2018) 138: 490-499; Kleszcynski, K. et al. Dermato-endocrinology (2011) 3: 27-31].

It has been shown that light can entrain the circadian clock in these peripheral tissues even after SCN ablation or scarring, although oscillations were found to be lower in these cases [Id., citing Buhr, E D et al. Curr. Biol. (2019) 29: 3478-3487; Welz, P S et al. Cell (2019) 177: 1436-1447; Koronowski, K B et al. Cell (2019) 177: 1448-1462]. The peripheral clocks also can communicate with each other, achieving entrainment independent of the SCN. The potential of UV, visible, and infra-red light to cause DNA damage, oxidative stress, lipid peroxidation, etc., in skin cells has been well characterized. Furthermore, UV light is also able to stimulate the production of melanin and melanocyte stimulating hormone (MSH) in the skin. The knowledge that skin cells have been known to interact with light makes it even more plausible that they can be directly entrained by light exposure.

### Influence of the Circadian Clock on the Immune Response of the Skin

The components of the circadian clock machinery are crucial to the development and functioning of a robust immune system. Indeed, most immune cell lineages have intrinsic clocks that govern their maturation, migration, differentiation, and function. An example is NFIL3, which is responsible for the development and maintenance of a population of interferon-gamma (IFN-7) producing group 1 innate lymphoid cells (ILC-1s) and NK cells [Id., citing Greenberg, E N et al. Proc. Natl Acad. Sci. USA (2020) 117: 5761-5771].

This, in turn, can be linked to the rhythmic activation of IFN-sensitive gene pathways in the skin, including a key transcription factor IFN regulatory factor 7 (Irf7) via Toll-like receptor 7 (TLR7). Similarly, ROR-α expression is thought to be increased in activated phenotype Treg cells in mouse skin [Id., citing Malhotra, N. et al. Sci. Immunol. (2018) 3: eaao6923]. Activated Treg cells expressing ROR-α have been shown to attenuate the function of group 2 innate lymphoid cells (ILC-2s) that reside in the skin. This has been shown to limit allergic skin inflammation in models of atopic dermatitis mediated by type II cytokines including IL-4, IL-5, and IL-13.

The circadian clock determines the rhythm with which immune cells circulate or migrate into tissues. The adhesion molecules ICAM-1 and VCAM-1 on endothelial cells vary based on the degree of inflammation, as well as rhythmicity, and act as homing signals for leukocytes in homeostasis, as well as inflammation [Id., citing He, W. et al. Immunity (2018) 49: 1175-1190]. Moreover, in skin, CD44 appears to be the adhesion molecule that varies in a circadian manner and acts as a honing signal for leucocytes in endothelial cells that constitute the capillaries of the dermis [Id., citing He, W. et al. Immunity (2018) 49: 1175-1190; Zoller, M. et al. J. Leukoc. Biol. (2007) 82: 57-71]. This circadian rhythmic variation prevents overactivation of the immune system when an external challenge is unlikely, and preparation of the immune system in more active phases of the day when a host is more likely to be faced with a challenge of a pathogen [Id, citing He, W. et al. Immunity (2018) 49: 1175-1190].

Although the immune system remains constantly vigilant and primed to mount a response to antigens, research into the influence of circadian rhythm on the immune system suggests an existence of a partition of the day into two phases. The first phase is one of heightened vigilance during waking hours where most activity occurs and an immune onslaught is most likely. This is followed by a recovery phase where resolution of inflammation and tissue repair occurs in the entire organism including the skin [Id, citing Curtis, A M et al. Immunity (2014) 40: 178-186].

The modulation of the two immunological pathways in skin has been shown to be influenced by glucocorticoids and nutrient intake, which act as zeitgebers. Of these, glucocorticoids have been studied in greater depth and have been linked to the central clock in the SCN [Id, citing San Phan, T. et al. J. Sci. Adv. (2021) 7: eabe0337; Palomino-Segura, M. and Hidalgo, A. J. Exp. Med. (2021) 218: e20200798; Waggoner, SN, Curr. Allergy Asthma Rep. (2020) 20: 2; Oster, H. et al. Endocr. Rev. (2016) 38: 3-45].

The secretion of adrenocorticotropin (ACTH) from the anterior pituitary gland is under the control of the SCN [Id, citing Curtis, A M et al. Immunity (2014) 40: 178-186]. However, ablation of the adrenal glands does not lead to loss of circadian oscillation in the skin and other peripheral tissue. In addition to the fact that skin can be directly entrained, this retention of circadian rhythm could be explained by the fact that keratinocytes of the epidermal layer in the skin are capable of regulating immune function by de novo synthesis of glucocorticoids via 118-hydroxylase (Cyp11b1), in addition to reactivation of inactive glucocorticoids via the enzyme 118-hydroxysteroid dehydrogenase type 1 (HSD11B1) [Id, citing San Phan, T. et al. J. Sci. Adv. (2021) 7: eabe0337; Buhr, E D et al. Curr. Biol. (2019) 29: 3478-3487].

Inflammation can disrupt the local, peripheral circadian clocks, as well as the central clock in the SCN. Recent reports implicate the NF-kB pathway in playing a central role in causing these perturbations [Id, citing Shen, Y. et al. PLoS Genet. (2021) 17: e1009933; Hong, H K et al. Genes Dev. (2018) 32: 1367-1379; Haspel, J A, et al. Nat. Commun. (2014) 5: 4753]. In addition to this pathway, researchers have shown that TNF-α, IFN-7, IL-1, and LPS are capable of disrupting the oscillations of a core clock gene and the genes that they control [Id., citing Curtis, A M et al. Immunity (2014) 40: 178-186; Waggoner, SN. Curr. Allergy Asthma Rep. (2020) 20: 2; Abreu, M. et al. Sci. Rep. (2018) 8: 11474; Yoshida, K. et al. Scand. J. Rheumatol. (2013) 42: 276-280; Bashir, M M et al. Arch. Dermatol. Res. (2009) 301: 87-91; Cavadini, G. et al. Proc. Natl Acad. Sci. USA (2007) 104: 12843-12848; Cermakian, N., et al. Chronobiol. Int. (2013) 30: 870-888; Castanon-Cervantes, O. et al. J. Immunol. (2010) 185: 5796-5805; Okada, K. et al. J. Surg. Res. (2008) 145: 5-12].

In humans, epidemiological studies have also associated shift work, where the circadian clock is assumed to be disrupted, with higher risk of psoriasis [Id., citing Paganelli, R. et al. Clin. Mol. Allergy (2018) 16: 1; Wu, G. et al. Proc. Natl Acad. Sci. USA (2018) 115: 12313-12318]. These inflammatory reactions brought on by circadian disruptions are also likely to compromise the integrity of the skin, since, in human keratinocytes, TIMP3, which is a broad spectrum inhibitor of extracellular matrix (ECM)-degrading enzymes (MMPs, ADAM, ADAMTS), is likely to be under CLOCK control [Id., citing Sherratt, M J et al. Matrix Biol. (2019) 84: 97-110; Palomino-Segura, M. and Hidalgo, A. J. Exp. Med. (2021) 218: e20200798; Waggoner, SN. Curr. Allergy Asthma Rep. (2020) 20: 2; Scheiermann, C. et al. Nat. Rev. Immunol. (2018) 18: 423-437; Matsui, M S et al. Int. J. Mol. Sci. (2016) 17: 801; Fan, D. and Kassiri, Z. Front. Physiol. (2020) 11: 661; Park, S. et al. FASEB J. (2018) 32: 1510-23; Yeom, M. et al. Molecules (2018) 23: 745]. Thus, the magnitude of immune responses in the skin are profoundly impacted by the circadian system. In turn, disruption of the circadian rhythm of the skin leads to immune hyperactivity or an aberrant immune response that can manifest as pathologies such as dermatitis or psoriasis.

### Influence of the Circadian Clock on Skin Homeostasis and Stress Mediation

Circadian oscillations are observed in keratinocytes and melanocytes of the epidermis and the fibroblasts of the dermis [Id., citing Sandu, C. et al. Cell. Mol. Life Sci. (2015) 72: 2237-2248; Sandu, C. et al. Cell Mol. Life Sci. (2012) 69: 3329-3339]. The circadian clock machinery responsible for oscillations impact the metabolic processes of these cells and has an impact of tissue homeostasis. The epidermis is generated from epidermal stem cells in the basal layer that undergo asymmetric cell division, giving rise to either daughter stem cells or keratinocytes that will undergo a process of differentiation and desquamation to form the horny layer of the stratum corneum. It takes approximately 14 days for epidermal stem cells to end up as part of the stratum corneum.

During this 2-week period, the process of differentiation does not occur continuously but instead appears to occur in five sequential 24-h cyclic phases coordinated by the circadian clock. When studied via gene expression, each phase lasts for 4-5 h [Id., citing Sherratt, M J et a. Matrix Biol. (2019) 84: 97-110; Janich, P. et al. Cell Stem Cell (2013) 13: 745-753; Eckhart, L. et al. Biochim. Biophys. Acta (2013) 1833: 3471-3480]. In differentiated keratinocytes, the genes upregulated in the first three phases under circadian control remained similar to their undifferentiated counterparts but included genes associated with DNA damage protection and repair, indicating constant vigilance against assault to the genetic code.

In the next two phases, differentiated keratinocytes seem to shift their focus to building a defensive barrier with genes for differentiation and keratin organization being upregulated. This is likely to include the surface lipids of the skin that are under clock control and contribute to the skin barrier [Id., citing Janich, P. et al. Cell Stem Cell (2013) 13: 745-753; Jia, Y et al. Exp. Dermatol. (20019) 28: 858-862]. The differentiation process is not only dependent on the expression of the clock genes, but also their amplitude.

Melanocytes and dermal fibroblasts have also been shown to possess functioning circadian clock machinery, but the amplitude of oscillations appears smaller than that of keratinocytes. Despite this, the circadian clock plays a functional role in melanocytes by controlling the abundance of melanosomes, as well as the expression of melanin synthesis enzyme, Tyrosinase and the phosphorylation of melanocyte inducing transcription factor (MITF), which increases when BMAL1 or PER1 are silenced [Id., citing Hardman, J A et al. J. Investig. Dermatol. (2015) 135: 1053-1064; Slominski, A T et al. J. Investig. Dermatol. (2015) 135: 943-945].

The protein OPN4 (Melanopsin) has been shown to affect the molecular clock components and their responsiveness to classical clock activators in melanocytes. Knocking out OPN4 in melanocytes resulted in rapid cell cycle progression and increased cellular proliferation, which correlated with the altered gene expression of MITF and the core circadian clock components [Id., citing De Assis, L V M et al. Curr. Issues Mol. Biol. (2021) 43: 1436-1450]. The impact of the function of the circadian clock on dermal fibroblasts is yet to be characterized. However, the influence of circadian rhythm on the synthesis and secretion of Type I collagen, a major component of the ECM of the dermis, is already known [Id., citing Chang, J. et al. Nat. Cell Biol. (2020) 22: 74-86]. Furthermore, the efficiency of migration and adhesion of fibroblasts modulated via actin dynamics was found to be circadian regulated [Id., citing Hoyle, N P et al. Sci. Transl. Med. (2017) 9: eaa12774].

Not only is normal function of skin impacted by circadian oscillations, but also the ability of skin to deal with stress is influenced by the cellular clock. As described above, the genes involved in DNA damage protection and repair in the epidermis are under clock control, as well as genes that mediate oxidative stress responses, in particular NRF2, the peroxiredoxins, glutathione peroxidase, and sestrins [Id., citing Ishii, T. et al. Free Radic. Biol. Med. (2018) 119: 34-44; Ndiaye, M A et al. Antioxid. Redox. Signal. (2014) 20: 2982-2996; Edgar, R S et al. Nature 92012]485: 459-64; Avitabile, D. et al. Intl J. Biochem. Cell Biol. (2014) 53: 24-34; Kolinjivadi, A M et al. J. Endocr. Re. at. Cancer (2021) 28: R55-R66; Kondratov, R V et al. Aging (2009) 1: 979-987].

In human skin, the activity of the DNA repair enzyme 8-oxoguanine DNA glycosylase (OGG1) was higher at night [Id., citing Manzella, N. et al. Sci. Rep. (2015) 5: 13752]. This is particularly important in the case of melanocytes, since they can accumulate DNA damage long after UV exposure, via melanin excitation [Id., citing Lyons, A B et al. J. Clin. Aesthet. Dermatol. (2019) 12: 42-45; Premi, S. et al. Science (2015) 347: 842-847].

The evolutionary conserved hormone melatonin is also responsible for combating DNA damage and oxidative stress, as well as maintaining skin homeostasis [Id., citing Slominski, A T et al. J. Investig. Dermatol. (2018) 138: 490-499]. This correlates with the finding that the circadian rhythm of melanin secretion is disrupted in psoriatic patients [Id., citing Mozzanica, N. et al. Acta Derm. Venereol. (1988) 68: 312-16]. Since this molecule and its related metabolites are free radicle scavengers, it is capable of stress mediation [Id., citing Slominski, A T et al. J. Investig. Dermatol. (2018) 138: 490-499; Ndiaye, M A et al. Antiox. Redox Signal. (2014) 20: 2982-2996].

Skin pigmentation and hair growth are also controlled by melatonin; its activity may also be influenced by the skin's circadian clock [Id., citing Hardman, J A et al. J. Investig. Dermatol. (2015) 135: 1053-1064; Slominski, A T et al. J. Investig. Dermatol. (2015) 135: 943-45; Al-Nuaimi, Y. et al. J. Investig. Dermatol. (2014) 134: 610-619].

Melatonin has also been implicated in the control of skin and body temperature in a circadian manner. In rat skin, the circadian clock machinery of dermal fibroblasts is capable of using melatonin as an internal signal to fine-tune its oscillations, with temperature being used as an external queue [Id., citing Cuesta, M. et al. J. Biol. Rhythms (2017) 32: 257-273].

### The Circadian Clock and Seasonality

Physiological changes in human skin are a consequence of seasonality triggered by several parameters such as light irradiation of various wavelengths with changing intensities over the year, temperature, temperature shifts between indoors and outdoors, humidity, and sweat resulting in, for example, a decrease in pH due to acidification and wind accelerated evaporation, thus modulating trans-epidermal water loss (TEWL). The seasonal temperature changes modulate blood microcirculation and thus the accessibility to nutrients, which contributes to physiological skin changes.

### Intersection of Circadian Rhythms and Aging

The role of circadian rhythm and its relationship with skin aging is only now beginning to be understood. The rhythmicity of multiple features are altered with age. This includes sleep, body temperature cycles, and locomotor activity [Id., citing Duan, J. et al. FEBS Lett. (2021) 595: 2413-2436; Hood, S. and Amir, S. J. Clin. Investig. (2017) 127: 437-446]. For circadian rhythm, the amplitude of oscillation of clock controlled genes decreases with age in peripheral tissue, but not in the SCN, particularly when Per2 is being tracked [Id., citing Buijink, M R et al. J. Biol. Rhythms (2020) 35: 167-179]. This indicates that age-related circadian alteration in peripheral clocks are independent of the SCN clock. In aged skin, this problem is further compounded by the presence of senescent cells. Senescent cells show dampened circadian rhythmicity and are less efficient in the transmission of circadian signals to their clocks [Id., citing Kunieda, T. et al. Circ. Res. (2006) 98: 532-539].

Therefore, senescence is implicated as the mechanism by which aging impairs entrainment of peripheral circadian clocks [Id., citing Kunieda, T. et al. Cir. Res. (2006) 98: 532-539; Ahmed, R. et al. Front. Neurosci. (2021) 15: 638122]. Furthermore, aged dermal fibroblasts secrete a unique aging-associated set of proteins, distinct from the canonical senescence-associated secretory phenotype. These include multiple candidates involved in inflammatory signaling and maintenance or alteration of the tissue microenvironment [Id., citing Waldera Lupa, D M et al. J. Investig. Dermatol. (2015) 135: 195419-68]. Thus, there is a high likelihood that aging via senescence is directly capable of disrupting the above mentioned immunological and stress mediatory pathways in skin. Since the rigidity of the tissue microenvironment is also impacted, aging also likely dampens the circadian clock of keratinocytes (which prefer a softer matrix) and fibroblasts (which prefer a firmer matrix) [Id, citing Williams, J. et al. J. Cell Sci. (2018) 131: jcs208223; Yang, N. et al. Nat. Commun. (2017) 8: 14287].

The circadian oscillations of epidermal cells remain robust even under aged conditions, but they are rewired to adapt to the stressors associated with an aged environment and remain committed to development and maintenance of the skin barrier through daily rhythmic cell division, in spite of DNA damage they many have incurred [Id, citing Solanas, G. et al. Cell (2017) 170: 678-692].

### Plant-Derived Exosomes Affect a Human Keratinocyte Cell Line In Vitro

Plant-derived secondary metabolites are natural substances including alkaloids, flavonoids, polyphenols, terpenoids, and quinones; these are widely used as cosmeceuticals (cosmetic products that contain bioactive ingredients with potential therapeutic benefits for the skin) because they exert beneficial effects on the human skin, such as antiaging, moisturizing, whitening, regeneration, and nutritional supply [Cho, J H et al. Applied Biological Chemistry (2022) 65: 8].

Typical examples are ginseng (Panax ginseng) and green tea (Camellia sinensis). Ginseng has several ginsenosides as representative active ingredients and exerts antiaging, anti-inflammatory, and antioxidative effects [Id, citing Kang, T H et al. J. Ethnopharmacol. (2009) 123: 446-451; Hong, C E and Lyu, SY. Immune Netw. (2011) 11: 42-49; Park, H J et al. J. Ginseng Res. (2012) 36: 225-414-415]. Green tea has reportedly been effective in antioxidation, photoprotection, and improvement in skin-related conditions owing to the active component of flavonoids including catechin and polyphenols [Id, citing Yarnell, E. and Abascal, K. Altern. Compl. Ther. (2012) 18: 141-144; Meetham, P. et al. Rev. Bras. Farmacogn. (2018) 28: 214-217; Koch, W. et al. Molecules (2019) 24: 4277].

To assess whether plant exosomes exert effects on human cells, extracts and exosomes from ginseng and green tea were used to treat keratinocytes in vitro and the change in transcriptosomes was compared to analyze the differential effect on cells. To prepare plant extracts, the leaf part of Green tea (1 kg) and the root part of Ginseng plants were hot air dried at 45 °C for 24 h, finely ground using a blender, and 25 g then extracted in 25 L of distilled water at 80 °C for 3 hr and filtered through a 0.45 um mesh filter. Exosomes were prepared by ultracentrifugation or an aqueous two-phase extraction system (ATPS).

A Human Epidermal Keratinocyte (HaCaT) cell line was cultured in Dulbecco Modified Eagles Medium (DMEM) supplemented with 10% fetal bovine serum (FBS), 1% penicillin and 1% streptomycin at 37 C, 5% CO₂. Keratinocytes were seeded at a density of 1×105 in a 35 mm cell culture plate and incubated for 24 h. After incubation, cells were treated with fresh media containing 2% of a water extract or 1×10E8/mL plant exosomes and mock-treated with distilled water as a control for 6 h. Extracts are normally treated for >24 h, so this 6 hr treatment window was shorter than normally required. Experiments were conducted when cell density reached 70-80% confluency. Exosomes showed a Gaussian distribution of nanoparticles within the range of 87-226 nm, inclusive, for ginseng and 108-234 nm, inclusive, for green tea.

The exosome treatment group had significantly higher numbers of expressed genes than the extract treatment or control groups. In the extract treatment group, 386 genes and 420 genes were upregulated for ginseng and green tea, respectively and 390 and 460 genes were downregulated. In the exosome treatment group, 877 and 843 genes were upregulated and 658 and 620 genes were downregulated for ginseng and green tea, respectively. This result implies that exosomes were effectively absorbed to keratinocytes. A previous study had reported that cabbage-derived exosomes were effectively absorbed to human cells by penetrating into them [Id., citing You, J Y et al. Bioact. Mat. (2021) 6: 4321-4332].

Expression patterns of genes related to skin aging, regenerating, barriers, and moisturizing were analyzed. Gene expression level of MMP12, MMP13, and NOTCH3, which are associated with skin aging, reportedly increase when exposed to light stress such as UVB and cause decomposition of collagen and skin aging [Id., citing Tewari, A. et al. J. Invest. Dermatol. (2014) 134: 2598-2609; Pittayapruek, P. et al. Int. J. Mol. Sci. (2016) 17: 868; Rossi, M. and Abdelmohsen, K. Cells (2021) 10: 1740].

Expression of these genes significantly decreased in the exosome treatment groups of ginseng and green tea, with no notable changes in the extract treatment groups. However, a previous study reported that the gene expression was decreased by more than twofold change when keratinocytes were treated with compound K, obtained from ginseng extract, for 24 h [Id., citing Kim, S. et al. Biochem. Biophys. Res. Commun. (2004) 316: 348-55]. Analyzing the expression of genes related to skin regeneration revealed that fibroblast growth factor 12 (FGF12), which suppresses radiation damage to skin tissue, increased specifically in the ginseng exosome treatment group [Id., citing Nakayama, F. et al. J. Biol. Chem. (2011) 286: 25823-34]. A gene encoding a heparin sulfate sulfotransferase enzyme (HS3ST3A1), which facilitates skin regeneration by affecting skin regeneration signal delivery, and a lysyl oxidase (LOX) gene, which considerably influences healing of skin wounds by facilitating ECM stabilization through ECM formation, development, maturation and remodeling, showed an increase in the exosome treatment group [Id., citing Patel, V N et al. Dev. Cell (2014) 29: 662-673; Cai, L. et al. Tissue Eng. Regen. Med. (2017) 14: 15-30].

A vimentin (VIM) gene, which affects the differentiation and migration of keratinocytes; ELOVL3, whose protein product belonged to a highly conserved family of microsomal enzymes involved in the formation of very long chain fatty acids (VLCFA), which affects lipid acid biosynthesis; and a keratin 1 (KRT1) gene whose protein product is a fibrillary protein that form skin, hair, and nails, are all recognized as genes that construct skin barrier and prevent water loss [Id., citing Velez-delValle, C. et al. Sci. Rep. (2016) 6: 24389; Cheng, F. et al. Proc. Nat. Acad. Sci. USA (2016) E4320-E4327; Westerberg, R. et al. J. Biol. Chem. (2004) 279: 5621-5629; Roth, W. et al. J. Cell Sci. (2012) 125: 5269-5279]. These genes responded particularly to the exosome treatment group and showed the highest increase in response to ginseng exosomes.

### The Medicinal Plant Aloe Vera: Pharmacological Properties

Aloe vera (Aloe barbadensis Miller, family Xanthorrhoeaceae) is a perennial green herb with bright yellow tubular flowers that is extensively distributed in hot and dry areas of North Africa, the Middle East of Asia, the Southern Mediterranean, and the Canary Islands. The colorless mucilaginous gel from Aloe vera leaves has been extensively used for pharmacological and cosmetic applications. Traditionally, this medicinal plant has been employed to treat skin problems (burns, wounds, and anti-inflammatory processes).

Moreover, Aloe vera has shown other therapeutic properties including anticancer, antioxidant, antidiabetic, and antihyperlipidemic effects. Aloe vera contains more than 75 different compounds, including vitamins (vitamin A, C, E, and B12), enzymes (i.e., amylase, catalase, and peroxidase), minerals (i.e., zinc, copper, selenium, and calcium), sugars (monosaccharides such as mannose-6-phosphate and polysaccharides such as glucomannans), anthraquinones (aloin and emodin), fatty acids (i.e., lupeol and campesterol), hormones (auxins and gibberellins), and others (i.e., salicylic acid, lignin, and saponins) [Sanchez, M. et al. Molecules (2020) 25: 1324, citing Surjushe, A. et al. Indian J. Dermatol. (2008) 53: 163-166; Malik, I and Zarnigar, H N. Int. Res. J. Phar. (2003) 4: 75-79; Maan, A A et al. J. Herb. Med. (2018) 12: 1-10].

### Skin Protection

Most in vitro studies on skin protection study the ability of Aloe vera and active compounds in wound healing. The immortalized human keratinocyte HaCaT cell line, the primary normal human epidermal keratinocytes HEKa cell line, and fibroblast cell lines are the most used. These studies have revealed that Aloe vera and its major compounds (aloesin, aloin, and emodin) exert their protective action mainly through antioxidant and anti-inflammatory mechanisms.

The most common models for in vivo studies are genetically modified animals (BALB/c mice, HR-1 hairless mice and SKH-1 hairless mice) and UV and X-ray skin damage in animals. Most of these in vivo studies have been done with Aloe vera extracts and gel. Application of topical Aloe vera favored wound healing in animal models with dermal incisions by reducing inflammatory cell infiltration, increasing CD4+/CD8+ ratio lymphocytes, and improving epidermal thickness and collagen deposition [Id., citing Brandao, M L et al. Acta Cir. Bras. (2016) 31: 570-577; Oryan, A. et al. Annals Plast. Sug. (2016) 77: 37-46; Takzaree, N. t al. J. Physiol. Pharm. (2016) 94: 1285-90; Yos Adi Prakoso, K. J. Trop. Med. (2018) 2018: 6218303].

Clinical trials have demonstrated that Aloe vera facilitated rapid tissue epithelialization and granulation in burns [Id, citing Irani, P S and Varaie, S. Iran. J. Med. Sci. (2016) 41: S3], promoted healing of cesarean wounds [Id, citing Molazem, Z. et al. Global J. Health Sci. (2015) 7: 203], and accelerated wound healing of split-thickness skin graft donor sites [Id, citing Burusapat, C. et al. Plast. Reconstr. Surg. (2018) 142: 217-236]. Furthermore, Aloe vera has been investigated in randomized, double-blind, placebo-controlled studies for its benefits to maintain healthy skin.

The daily oral intake of 40 μg of Aloe sterol (cycloartenol and lophenol) for at least 12 weeks improved skin elasticity in men under 46 years exposed to the sunlight but who do not use sunscreen to protect themselves [Id, citing Tanaka, M. et al. Clin. Cosmet. Invest. Dermat. (2016) 9: 435-442], reduced facial wrinkles in Japanese women over 40 years old by stimulating hyaluronic acid and collagen production [Id., citing Tanaka, M. et al. Clinical Cosmet. Invest. Dermat. (2015) 8: 95-104], and increased gross elasticity, net elasticity, and biological elasticity in women aged 30-59 [Id., citing Tanaka, M. et al. Skin Pharmacol. Physiol. (2016) 29: 309-317].

### Anti-Inflammatory and Anti-Oxidant Activity

Most recent studies on anti-inflammatory activity of Aloe vera are focused on the action mechanism of isolated compounds in murine macrophage RAW264.7 cells and mice stimulated with LPS. Hence, the potential anti-inflammatory effect of aloin was reported to be related to its ability to inhibit cytokines, ROS production, and the JAK1-STAT1/3 signaling pathway [Id., citing Ma, Y. et al. Int. J. Mol. Med. (2018) 42: 1925-1934; Jiang, K. et al. Int. Immunopharm. (2018) 64: 140-50]. Moreover, aloe-emodin sulfates/glucuronides (0.5 μM), rhein sulfates/glucuronides (1.0 μM), aloe-emodin (0.1 M), and rhein (0.3 M) inhibited pro-inflammatory cytokines and nitric oxide production, iNOS expression, and MAPKs phosphorylation [Id., citing Li, C Y et al. Am. J. Chin. Med. (2017) 45: 847-861].

The antioxidant activity of Aloe vera has been attributed, at least in part, to anthraquinones and related compounds (10 μM) which possess peroxyl radical scavenging activity and reducing capacity [Id., citing Sun, Y N et al. Nat. Prod. Res. (2017) 31: 2810-2813].

Characterization of Heat-Stressed Aloe vera Plant-Derived Exosome-Like Nanoparticles

As previously described in U.S. Non-Provisional application Ser. No. 19/270,704, plant-derived extracellular vesicles (EVs), including exosome-like nanoparticles derived from Aloe vera, have been investigated for their potential biological activity and utility in therapeutic and cosmetic applications. In that work, Aloe vera plants were cultivated under controlled environmental conditions and subjected to heat shock to modulate stress response pathways and associated vesicular cargo.

Specifically, plants maintained at approximately 25 °C were exposed to elevated temperatures ranging from about 35 °C to about 45 °C for defined periods of time. Inner leaf pulp comprising parenchymal mesophyll cells was isolated, homogenized, and processed to obtain vesicle-containing fractions. EVs were isolated from the homogenate using dilution in phosphate-buffered saline (PBS) followed by differential centrifugation.

Analysis of vesicles obtained from heat stressed plants indicated that environmental stress conditions can influence vesicular cargo, including nucleic acid content. For example, total RNA isolated from EVs derived from heat-shocked plants (35 °C heat shock for 2 hours, 45 °C heat shock for 3 hours, and 24 hour room temperature recovery after 45 °C heat shock for 3 hours) demonstrated increased levels of stress-associated transcripts, including HSP70, relative to EVs derived from non-stressed plants.

The data showed a 10-fold increase in transcript level of the stress response molecule HSP70 after a heat shock of 35 °C in comparison to the same for non-heat shocked exosomes (control). The transcript level of HSP70 after a heat shock of 45 °C was about 17-fold higher than that for non-heat shocked exosomes (control), and transcript level of HSP70 after heat shock of 45 °C with a 24 hour waiting period was about 22-fold higher than that for non-heat shocked exosomes (control) (FIG. 4). These observations suggested that stress conditions may be used to modulate the molecular composition of plant-derived EVs.

Further characterization of vesicular cargo using RNA sequencing identified a signature miRNA cargo in Aloe-derived EVs (Table 2). Comparative analyses indicated differential expression of miRNAs, and downstream pathway analyses suggested that such vesicular cargo may be associated with biological pathways relevant to cellular signaling, proliferation, and immune response (Table 3).

TABLE 2
Aloe-Derived Exosome Signature miRNAs

| | | SEQ | |
|---|---|---|---|
| | | ID | miRBase |
| Signature miRNA | Mature miRNA Sequence | NO: | Accession |
| ath-miR166a-3p | UCGGACCAGGCUUCAUUCCCC | 1 | MIMAT0000189 |
| ath-miR166b-3p | UCGGACCAGGCUUCAUUCCCC | 2 | MIMAT0000190 |
| ath-miR166e-3p | UCGGACCAGGCUUCAUUCCCC | 3 | MIMAT0000193 |
| ath-miR396a-5p | UUCCACAGCUUUCUUGAACUG | 4 | MIMAT0000944 |
| ath-miR396b-5p | UUCCACAGCUUUCUUGAACUU | 5 | MIMAT0000945 |
| ath-miR156f-5p | UGACAGAAGAGAGUGAGCAC | 6 | MIMAT0000171 |
| ath-miR168b-5p | UCGCUUGGUGCAGGUCGGGAA | 7 | MIMAT0000199 |
| ath-miR156c-5p | UGACAGAAGAGAGUGAGCAC | 8 | MIMAT0000168 |
| ath-miR162a-3p | UCGAUAAACCUCUGCAUCCAG | 9 | MIMAT0000182 |
| ath-miR162b-3p | UCGAUAAACCUCUGCAUCCAG | 10 | MIMAT0000183 |
| ath-miR396a-3p | GUUCAAUAAAGCUGUGGGAAG | 11 | MIMAT0031908 |
| ath-miR168a-5p | UCGCUUGGUGCAGGUCGGGAA | 12 | MIMAT0000198 |
| ath-miR156b-5p | UGACAGAAGAGAGUGAGCAC | 13 | MIMAT0000167 |
| ath-miR156a-5p | UGACAGAAGAGAGUGAGCAC | 14 | MIMAT0000166 |
| ath-miR156d-5p | UGACAGAAGAGAGUGAGCAC | 15 | MIMAT0000169 |
| ath-miR164c-5p | UGGAGAAGCAGGGCACGUGCG | 16 | MIMAT0001017 |
| ath-miR408-3p | AUGCACUGCCUCUUCCCUGGC | 17 | MIMAT0001011 |
| ath-miR165a-3p | UCGGACCAGGCUUCAUCCCCC | 18 | MIMAT0000187 |
| ath-miR160a-5p | UGCCUGGCUCCCUGUAUGCCA | 19 | MIMAT0000178 |
| ath-miR157b-5p | UUGACAGAAGAUAGAGAGCAC | 20 | MIMAT0000173 |
| ath-miR157a-5p | UUGACAGAAGAUAGAGAGCAC | 21 | MIMAT0000172 |
| ath-miR164b-5p | UGGAGAAGCAGGGCACGUGCA | 22 | MIMAT0000186 |
| ath-miR5016 | UUCUUGUGGAUUCCUUGGAAA | 23 | MIMAT0020520 |
| ath-miR5998b | ACAGUUUGUGUUUUGUUUUGU | 24 | MIMAT0023522 |
| ath-miR5020a | UGGAAGAAGGUGAGACUUGCA | 25 | MIMAT0020524 |
| ath-miR836 | UCCUGUGUUUCCUUUGAUGCGUGG | 26 | MIMAT0004257 |
| ath-miR158a-5p | CUUUGUCUACAAUUUUGGAAA | 27 | MIMAT0031873 |
| ath-miR395e | CUGAAGUGUUUGGGGGAACUC | 28 | MIMAT0000942 |
| ath-miR402 | UUCGAGGCCUAUUAAACCUCUG | 29 | MIMAT0001003 |
| ath-miR5662 | AGAGGUGACCAUUGGAGAUG | 30 | MIMAT0022439 |
| ath-miR5630a | GCUAAGAGCGGUUCUGAUGGA | 31 | MIMAT0022389 |
| ath-miR5630b | GCUAAGAGCGGUUCUGAUGGA | 32 | MIMAT0022399 |
| ath-miR3933 | AGAAGCAAAAUGACGACUCGG | 33 | MIMAT0018348 |
| ath-miR5998a | ACAGUUUGUGUUUUGUUUUGU | 34 | MIMAT0023521 |
| ath-miR172e-3p | GGAAUCUUGAUGAUGCUGCAU | 35 | MIMAT0001019 |
| ath-miR5024-3p | CCGUAUCUUGGCCUUGUCAUU | 36 | MIMAT0021048 |
| ath-miR447a-3p | UUGGGGACGAGAUGUUUUGUUG | 37 | MIMAT0002113 |
| ath-miR414 | UCAUCUUCAUCAUCAUCGUCA | 38 | MIMAT0001322 |
| ath-miR167a-3p | GAUCAUGUUCGCAGUUUCACC | 39 | MIMAT0031883 |
| ath-miR172b-5p | GCAGCACCAUUAAGAUUCAC | 40 | MIMAT0000204 |
| ath-miR5636 | CGUAGUUGCAGAGCUUGACGG | 41 | MIMAT0022396 |
| ath-miR824-3p | CCUUCUCAUCGAUGGUCUAGA | 42 | MIMAT0032024 |
| ath-miR172e-5p | GCAGCACCAUUAAGAUUCAC | 43 | MIMAT0031918 |
| ath-miR404 | AUUAACGCUGGCGGUUGCGGCAGC | 44 | MIMAT0001005 |
| ath-miR447b | UUGGGGACGAGAUGUUUUGUUG | 45 | MIMAT0002114 |
| ath-miR826b | UGGUUUUGGACACGUGAAAAU | 46 | MIMAT0035543 |
| ath-miR169g-5p | UGAGCCAAGGAUGACUUGCCG | 47 | MIMAT0000911 |
| ath-miR868-3p | CUUCUUAAGUGCUGAUAAUGC | 48 | MIMAT0004319 |
| ath-miR830-3p | UAACUAUUUUGAGAAGAAGUG | 49 | MIMAT0004248 |
| ath-miR169f-5p | UGAGCCAAGGAUGACUUGCCG | 50 | MIMAT0000910 |
| ath-miR828 | UCUUGCUUAAAUGAGUAUUCCA | 51 | MIMAT0004244 |
| ath-miR8182 | UUGUGUUGCGUUUCUGUUGAUU | 52 | MIMAT0032781 |
| ath-miR160a-3p | GCGUAUGAGGAGCCAUGCAUA | 53 | MIMAT0031874 |
| ath-miR5635d | UGUUAAGGAGUGUUAACGGUG | 54 | MIMAT0022405 |
| ath-miR399c-3p | UGCCAAAGGAGAGUUGCCCUG | 55 | MIMAT0000953 |
| ath-miR5635c | UGUUAAGGAGUGUUAACGGUG | 56 | MIMAT0022428 |
| ath-miR398a-3p | UGUGUUCUCAGGUCACCCCUU | 57 | MIMAT0000948 |
| ath-miR391-5p | UUCGCAGGAGAGAUAGCGCCA | 58 | MIMAT0000933 |
| ath-miR781a | UUAGAGUUUUCUGGAUACUUA | 59 | MIMAT0003940 |
| ath-miR157c-3p | GCUCUCUAUACUUCUGUCACC | 60 | MIMAT0031872 |
| ath-miR399b | UGCCAAAGGAGAGUUGCCCUG | 61 | MIMAT0000952 |
| ath-miR5014b | AUUUGUACACCUAGAUCUGUA | 62 | MIMAT0023524 |
| ath-miR5635b | UGUUAAGGAGUGUUAACGGUG | 63 | MIMAT0022418 |
| ath-miR779.2 | UGAUUGGAAAUUUCGUUGACU | 64 | MIMAT0004329 |
| ath-miR390b-5p | AAGCUCAGGAGGGAUAGCGCC | 65 | MIMAT0000932 |
| ath-miR833a-3p | UAGACCGAUGUCAACAAACAAG | 66 | MIMAT0004253 |
| ath-miR849 | UAACUAAACAUUGGUGUAGUA | 67 | MIMAT0004271 |
| ath-miR5635a | UGUUAAGGAGUGUUAACGGUG | 68 | MIMAT0022395 |
| ath-miR841b-3p | CAAUUUCUAGUGGGUCGUAUU | 69 | MIMAT0017742 |
| ath-miR390a-5p | AAGCUCAGGAGGGAUAGCGCC | 70 | MIMAT0000931 |
| ath-miR447c-3p | UUGGGGACGACAUCUUUUGUUG | 71 | MIMAT0002115 |
| ath-miR835-3p | UGGAGAAGAUACGCAAGAAAG | 72 | MIMAT0004256 |
| ath-miR5638b | ACAGUGGUCAUCUGGUGGGCU | 73 | MIMAT0022407 |
| ath-miR2112-3p | CUUUAUAUCCGCAUUUGCGCA | 74 | MIMAT0011155 |
| ath-miR5653 | UGGGUUGAGUUGAGUUGAGUUGGC | 75 | MIMAT0022425 |
| ath-miR166a-5p | GGACUGUUGUCUGGCUCGAGG | 76 | MIMAT0031880 |
| ath-miR159b-5p | GAGCUCCUUGAAGUUCAAUGG | 77 | MIMAT0031889 |
| ath-miR166b-5p | GGACUGUUGUCUGGCUCGAGG | 78 | MIMAT0031881 |
| ath-miR843 | UUUAGGUCGAGCUUCAUUGGA | 79 | MIMAT0004265 |
| ath-miR5015 | UUGGUGUUAUGUGUAGUCUUC | 80 | MIMAT0020519 |
| ath-miR781b | UUAGAGUUUUCUGGAUACUUA | 81 | MIMAT0022420 |
| ath-miR4245 | ACAAAGUUUUAUACUGACAAU | 82 | MIMAT0023523 |
| ath-miR169b-5p | CAGCCAAGGAUGACUUGCCGG | 83 | MIMAT0000906 |
| ath-miR5013 | UUUGUGACAUCUAGGUGCUUU | 84 | MIMAT0020517 |
| ath-miR864-5p | UCAGGUAUGAUUGACUUCAAA | 85 | MIMAT0004311 |
| ath-miR866-5p | UCAAGGAACGGAUUUUGUUAA | 86 | MIMAT0004315 |
| ath-miR5595a | ACAUAUGAUCUGCAUCUUUGC | 87 | MIMAT0023517 |
| ath-miR403-3p | UUAGAUUCACGCACAAACUCG | 88 | MIMAT0001004 |
| ath-miR164c-3p | CACGUGUUCUACUACUCCAAC | 89 | MIMAT0031916 |
| ath-miR835-5p | UUCUUGCAUAUGUUCUUUAUC | 90 | MIMAT0004255 |
| ath-miR165b | UCGGACCAGGCUUCAUCCCCC | 91 | MIMAT0000188 |
| ath-miR3434-5p | ACUUGGCUGAUUCUAUUAUU | 92 | MIMAT0017737 |
| ath-miR8176 | GGCCGGUGGUCGCGAGAGGGA | 93 | MIMAT0032775 |
| ath-miR5631 | UGGCAGGAAAGACAUAAUUUU | 94 | MIMAT0022390 |
| ath-miR399a | UGCCAAAGGAGAUUUGCCCUG | 95 | MIMAT0000951 |
| ath-miR4227 | UCACUGGUACCAAUCAUUCCA | 96 | MIMAT0017943 |
| ath-miR5666 | AUGGGACAUCGAGCAUUUAAU | 97 | MIMAT0022444 |
| ath-miR778 | UGGCUUGGUUUAUGUACACCG | 98 | MIMAT0003937 |
| ath-miR851-3p | UGGGUGGCAAACAAAGACGAC | 99 | MIMAT0004274 |
| ath-miR5663-3p | UGAGAAUGCAAAUCCUUAGCU | 100 | MIMAT0032129 |
| ath-miR832-3p | UUGAUUCCCAAUCCAAGCAAG | 101 | MIMAT0004251 |
| ath-miR5646 | GUUCGAGGCACGUUGGGAGG | 102 | MIMAT0022410 |
| ath-miR856 | UAAUCCUACCAAUAACUUCAGC | 103 | MIMAT0004300 |
| ath-miR837-5p | AUCAGUUUCUUGUUCGUUUCA | 104 | MIMAT0004258 |
| ath-miR846-3p | UUGAAUUGAAGUGCUUGAAUU | 105 | MIMAT0004269 |
| ath-miR827 | UUAGAUGACCAUCAACAAACU | 106 | MIMAT0004243 |
| ath-miR5633 | UAUGAUCAUCAGAAAACAGUG | 107 | MIMAT0022393 |
| ath-miR413 | AUAGUUUCUCUUGUUCUGCAC | 108 | MIMAT0001321 |
| ath-miR838 | UUUUCUUCUACUUCUUGCACA | 109 | MIMAT0004260 |
| ath-miR5654-5p | AUAAAUCCCAACAUCUUCCA | 110 | MIMAT0022426 |
| ath-miR172d-5p | GCAACAUCUUCAAGAUUCAGA | 111 | MIMAT0031901 |
| ath-miR5642a | UCUCGCGCUUGUACGGCUUU | 112 | MIMAT0022403 |
| ath-miR420 | UAAACUAAUCACGGAAAUGCA | 113 | MIMAT0001328 |
| ath-miR831-3p | UGAUCUCUUCGUACUCUUCUUG | 114 | MIMAT0004249 |
| ath-miR156d-3p | GCUCACUCUCUUUUUGUCAUAAC | 115 | MIMAT0031868 |
| ath-miR5018 | UUAAAGCUCCACCAUGAGUCCAAU | 116 | MIMAT0020522 |
| ath-miR8168 | AGGUGCUGAGUGUGCUAGUGC | 117 | MIMAT0032766 |
| ath-miR866-3p | ACAAAAUCCGUCUUUGAAGA | 118 | MIMAT0004316 |
| ath-miR8170-3p | UUGCUUAAAGAUUUUCUAUGU | 119 | MIMAT0032769 |
| ath-miR395b | CUGAAGUGUUUGGGGGGACUC | 120 | MIMAT0000939 |
| ath-miR395c | CUGAAGUGUUUGGGGGGACUC | 121 | MIMAT0000940 |
| ath-miR780.2 | UUCUUCGUGAAUAUCUGGCAU | 122 | MIMAT0003939 |
| ath-miR167c-5p | UAAGCUGCCAGCAUGAUCUUG | 123 | MIMAT0001018 |
| ath-miR393a-3p | AUCAUGCUAUCUCUUUGGAUU | 124 | MIMAT0031905 |
| ath-miR395f | CUGAAGUGUUUGGGGGGACUC | 125 | MIMAT0000943 |

TABLE 3
Differential Expression of miRNA in Exosomes Derived from Heat-
Stressed Aloe vera Plants vs. Non-Heat Stressed (Native) Aloe vera Plants

| | | | | Exosomes | Exosomes |
|---|---|---|---|---|---|
| | | | | Derived | Derived |
| | MiRBase | | SEQ | from Heat- | from |
| | Accession | | ID | Stressed | Native |
| miRNA | Number | Mature miRNA Sequence | NO: | Aloe vera | Aloe vera |
| ath- | MIMAT0020520 | UUCUUGUGGAUUCCUUGGA | 23 | + | − |
| miR5016 | | AA | | | |
| ath- | MIMAT0023522 | ACAGUUUGUGUUUUGUUUU | 24 | + | − |
| miR5998b | | GU | | | |
| ath- | MIMAT0020524 | UGGAAGAAGGUGAGACUUG | 25 | + | − |
| miR5020a | | CA | | | |
| ath- | MIMAT0004257 | UCCUGUGUUUCCUUUGAUG | 26 | + | − |
| miR836 | | CGUGG | | | |
| ath- | MIMAT0031873 | CUUUGUCUACAAUUUUGGA | 27 | + | − |
| miR158a- | | AA | | | |
| 5p | | | | | |
| ath- | MIMAT0000942 | CUGAAGUGUUUGGGGGAAC | 28 | + | − |
| miR395e | | UC | | | |
| ath- | MIMAT0001003 | UUCGAGGCCUAUUAAACCU | 29 | + | − |
| miR402 | | CUG | | | |
| ath- | MIMAT0022439 | AGAGGUGACCAUUGGAGAU | 30 | + | − |
| miR5662 | | G | | | |
| ath- | MIMAT0022389 | GCUAAGAGCGGUUCUGAUG | 31 | + | − |
| miR5630a | | GA | | | |
| ath- | MIMAT0022399 | GCUAAGAGCGGUUCUGAUG | 32 | + | − |
| miR5630b | | GA | | | |
| ath- | MIMAT0018348 | AGAAGCAAAAUGACGACUC | 33 | + | − |
| miR3933 | | GG | | | |
| ath- | MIMAT0023521 | ACAGUUUGUGUUUUGUUUU | 34 | + | − |
| miR5998 | | GU | | | |
| ath- | MIMAT0001019 | GGAAUCUUGAUGAUGCUGC | 35 | + | − |
| miR172e- | | AU | | | |
| 3 | | | | | |
| ath- | MIMAT0021048 | CCGUAUCUUGGCCUUGUCA | 36 | + | − |
| miR5024- | | UU | | | |
| 3p | | | | | |
| ath- | MIMAT0002113 | UUGGGGACGAGAUGUUUUG | 37 | + | − |
| miR447a- | | UUG | | | |
| 3p | | | | | |
| ath- | MIMAT0001322 | UCAUCUUCAUCAUCAUCGU | 38 | + | − |
| miR414 | | CA | | | |
| ath- | MIMAT0031883 | GAUCAUGUUCGCAGUUUCA | 39 | + | − |
| miR167a- | | CC | | | |
| 3p | | | | | |
| ath- | MIMAT0000204 | GCAGCACCAUUAAGAUUCA | 40 | + | − |
| miR172b- | | C | | | |
| 5p | | | | | |
| ath- | MIMAT0022396 | CGUAGUUGCAGAGCUUGAC | 41 | + | − |
| miR5636 | | GG | | | |
| ath | MIMAT0032024 | CCUUCUCAUCGAUGGUCUA | 42 | + | − |
| miR824- | | GA | | | |
| 3p | | | | | |
| ath- | MIMAT0031918 | GCAGCACCAUUAAGAUUCA | 43 | + | − |
| miR172e- | | C | | | |
| 5p | | | | | |
| ath- | MIMAT0001005 | AUUAACGCUGGCGGUUGCG | 44 | + | − |
| miR404 | | GCAGC | | | |
| ath- | MIMAT0002114 | UUGGGGACGAGAUGUUUUG | 45 | + | − |
| miR447b | | UUG | | | |
| ath- | MIMAT0035543 | UGGUUUUGGACACGUGAAA | 46 | + | − |
| miR826b | | AU | | | |
| ath- | MIMAT0000911 | UGAGCCAAGGAUGACUUGC | 47 | + | − |
| miR169g- | | CG | | | |
| 5p | | | | | |
| ath- | MIMAT0004319 | CUUCUUAAGUGCUGAUAAU | 48 | + | − |
| miR868-3 | | GC | | | |
| ath- | MIMAT0004248 | UAACUAUUUUGAGAAGAAG | 49 | + | − |
| miR830- | | UG | | | |
| 3p | | | | | |
| ath- | MIMAT0000910 | UGAGCCAAGGAUGACUUGC | 50 | + | − |
| miR169f- | | CG | | | |
| 5p | | | | | |
| ath- | MIMAT0004244 | UCUUGCUUAAAUGAGUAUU | 51 | + | − |
| miR828 | | CCA | | | |
| ath- | MIMAT0032781 | UUGUGUUGCGUUUCUGUUG | 52 | + | − |
| miR8182 | | AUU | | | |
| ath- | MIMAT0031874 | GCGUAUGAGGAGCCAUGCA | 53 | + | − |
| miR160a- | | UA | | | |
| 3p | | | | | |
| ath- | MIMAT0022405 | UGUUAAGGAGUGUUAACGG | 54 | + | − |
| miR5635d | | UG | | | |
| ath- | MIMAT0000953 | UGCCAAAGGAGAGUUGCCC | 55 | + | − |
| miR399c- | | UG | | | |
| 3p | | | | | |
| ath- | MIMAT0022428 | UGUUAAGGAGUGUUAACGG | 56 | + | − |
| miR5635c | | UG | | | |
| ath- | MIMAT0000948 | UGUGUUCUCAGGUCACCCC | 57 | + | − |
| miR398a- | | UU | | | |
| 3p | | | | | |
| ath- | MIMAT0000933 | UUCGCAGGAGAGAUAGCGC | 58 | + | − |
| miR391- | | CA | | | |
| 5p | | | | | |
| ath- | MIMAT0003940 | UUAGAGUUUUCUGGAUACU | 59 | + | − |
| miR781a | | UA | | | |
| ath- | MIMAT0031872 | GCUCUCUAUACUUCUGUCA | 60 | + | − |
| miR157c- | | CC | | | |
| 3p | | | | | |
| ath- | MIMAT0000952 | UGCCAAAGGAGAGUUGCCC | 61 | + | − |
| miR399b | | UG | | | |
| ath- | MIMAT0023524 | AUUUGUACACCUAGAUCUG | 62 | + | − |
| miR5014b | | UA | | | |
| ath- | MIMAT0022418 | UGUUAAGGAGUGUUAACGG | 63 | + | − |
| miR5635b | | UG | | | |
| ath- | MIMAT0004329 | UGAUUGGAAAUUUCGUUGA | 64 | + | − |
| miR779.2 | | CU | | | |
| ath- | MIMAT0000932 | AAGCUCAGGAGGGAUAGCG | 65 | + | − |
| miR390b- | | CC | | | |
| 5 | | | | | |
| ath | MIMAT0004253 | UAGACCGAUGUCAACAAAC | 66 | + | − |
| miR833a- | | AAG | | | |
| 3p | | | | | |
| ath- | MIMAT0004271 | UAACUAAACAUUGGUGUAG | 67 | + | − |
| miR849 | | UA | | | |
| ath- | MIMAT0022395 | UGUUAAGGAGUGUUAACGG | 68 | + | − |
| miR5635a | | UG | | | |
| ath- | MIMAT0017742 | CAAUUUCUAGUGGGUCGUA | 69 | + | − |
| miR841b- | | UU | | | |
| 3 | | | | | |
| ath- | MIMAT0000931 | AAGCUCAGGAGGGAUAGCG | 70 | + | − |
| miR390a- | | CC | | | |
| 5p | | | | | |
| ath- | MIMAT0002115 | UUGGGGACGACAUCUUUUG | 71 | + | − |
| miR447c- | | UUG | | | |
| 3p | | | | | |
| ath- | MIMAT0004256 | UGGAGAAGAUACGCAAGAA | 72 | + | − |
| miR835- | | AG | | | |
| 3p | | | | | |
| ath- | MIMAT0022407 | ACAGUGGUCAUCUGGUGGG | 73 | + | − |
| miR5638b | | CU | | | |
| ath- | MIMAT0011155 | CUUUAUAUCCGCAUUUGCG | 74 | + | − |
| miR2112- | | CA | | | |
| 3p | | | | | |
| ath- | MIMAT0022425 | UGGGUUGAGUUGAGUUGAG | 75 | + | − |
| miR5653 | | UUGGC | | | |
| ath- | MIMAT0031880 | GGACUGUUGUCUGGCUCGA | 76 | + | − |
| miR166a- | | GG | | | |
| 5p | | | | | |
| ath- | MIMAT0031889 | GAGCUCCUUGAAGUUCAAU | 77 | + | − |
| miR159b- | | GG | | | |
| 5p | | | | | |
| ath- | MIMAT0031881 | GGACUGUUGUCUGGCUCGA | 78 | + | − |
| miR166b- | | GG | | | |
| 5p | | | | | |
| ath- | MIMAT0004265 | UUUAGGUCGAGCUUCAUUG | 79 | + | − |
| miR843 | | GA | | | |
| ath- | MIMAT0020519 | UUGGUGUUAUGUGUAGUCU | 80 | + | − |
| miR5015 | | UC | | | |
| ath- | MIMAT0022420 | UUAGAGUUUUCUGGAUACU | 81 | + | − |
| miR781b | | UA | | | |
| ath- | MIMAT0023523 | ACAAAGUUUUAUACUGACA | 82 | + | − |
| miR4245 | | AU | | | |
| ath- | MIMAT0000906 | CAGCCAAGGAUGACUUGCC | 83 | + | − |
| miR169b- | | GG | | | |
| 5p | | | | | |
| ath- | MIMAT0020517 | UUUGUGACAUCUAGGUGCU | 84 | + | − |
| miR5013 | | UU | | | |
| ath- | MIMAT0004311 | UCAGGUAUGAUUGACUUCA | 85 | + | − |
| miR864- | | AA | | | |
| 5p | | | | | |
| ath- | MIMAT0004315 | UCAAGGAACGGAUUUUGUU | 86 | + | − |
| miR866- | | AA | | | |
| 5p | | | | | |
| ath- | MIMAT0023517 | ACAUAUGAUCUGCAUCUUU | 87 | + | − |
| miR5595a | | GC | | | |
| ath- | MIMAT0001004 | UUAGAUUCACGCACAAACU | 88 | + | − |
| miR403- | | CG | | | |
| 3p | | | | | |
| ath- | MIMAT0031916 | CACGUGUUCUACUACUCCA | 89 | + | − |
| miR164c- | | AC | | | |
| 3p | | | | | |
| ath- | MIMAT0004255 | UUCUUGCAUAUGUUCUUUA | 90 | + | − |
| miR835- | | UC | | | |
| 5p | | | | | |
| ath- | MIMAT0000188 | UCGGACCAGGCUUCAUCCC | 91 | + | − |
| miR165b | | CC | | | |
| ath- | MIMAT0017737 | ACUUGGCUGAUUCUAUUAU | 92 | + | − |
| miR3434- | | U | | | |
| 5p | | | | | |
| ath- | MIMAT0032775 | GGCCGGUGGUCGCGAGAGG | 93 | − | + |
| miR8176 | | GA | | | |
| ath- | MIMAT0022390 | UGGCAGGAAAGACAUAAUU | 94 | − | + |
| miR5631 | | UU | | | |
| ath- | MIMAT0000951 | UGCCAAAGGAGAUUUGCCC | 95 | − | + |
| miR399a | | UG | | | |
| ath- | MIMAT0017943 | UCACUGGUACCAAUCAUUC | 96 | − | + |
| miR4227 | | CA | | | |
| ath- | MIMAT0022444 | AUGGGACAUCGAGCAUUUA | 97 | − | + |
| miR5666 | | AU | | | |
| ath- | MIMAT0003937 | UGGCUUGGUUUAUGUACAC | 98 | − | + |
| miR778 | | CG | | | |
| ath- | MIMAT0004274 | UGGGUGGCAAACAAAGACG | 99 | − | + |
| miR851- | | AC | | | |
| 3p | | | | | |
| ath- | MIMAT0032129 | UGAGAAUGCAAAUCCUUAG | 100 | − | + |
| miR5663- | | CU | | | |
| 3p | | | | | |
| ath- | MIMAT0004251 | UUGAUUCCCAAUCCAAGCA | 101 | − | + |
| miR832- | | AG | | | |
| 3p | | | | | |
| ath- | MIMAT0022410 | GUUCGAGGCACGUUGGGAG | 102 | − | + |
| miR5646 | | G | | | |
| ath- | MIMAT0004300 | UAAUCCUACCAAUAACUUC | 103 | − | + |
| miR856 | | AGC | | | |
| ath- | MIMAT0004258 | AUCAGUUUCUUGUUCGUUU | 104 | − | + |
| miR837- | | CA | | | |
| 5p | | | | | |
| ath- | MIMAT0004269 | UUGAAUUGAAGUGCUUGAA | 105 | − | + |
| miR846- | | UU | | | |
| 3p | | | | | |
| ath- | MIMAT0004243 | UUAGAUGACCAUCAACAAA | 106 | − | + |
| miR827 | | CU | | | |
| ath- | MIMAT0022393 | UAUGAUCAUCAGAAAACAG | 107 | − | + |
| miR5633 | | UG | | | |
| ath- | MIMAT0001321 | AUAGUUUCUCUUGUUCUGC | 108 | − | + |
| miR413 | | AC | | | |
| ath- | MIMAT0004260 | UUUUCUUCUACUUCUUGCA | 109 | − | + |
| miR838 | | CA | | | |
| ath- | MIMAT0022426 | AUAAAUCCCAACAUCUUCC | 110 | − | + |
| miR5654- | | A | | | |
| 5p | | | | | |
| ath- | MIMAT0031901 | GCAACAUCUUCAAGAUUCA | 111 | − | + |
| miR172d- | | GA | | | |
| 5p | | | | | |
| ath- | MIMAT0022403 | UCUCGCGCUUGUACGGCUU | 112 | − | + |
| miR5642a | | U | | | |
| ath- | MIMAT0001328 | UAAACUAAUCACGGAAAUG | 113 | − | + |
| miR420 | | CA | | | |
| ath- | MIMAT0004249 | UGAUCUCUUCGUACUCUUC | 114 | − | + |
| miR831- | | UUG | | | |
| 3p | | | | | |
| ath- | MIMAT0031868 | GCUCACUCUCUUUUUGUCA | 115 | − | + |
| miR156d- | | UAAC | | | |
| 3p | | | | | |
| ath- | MIMAT0020522 | UUAAAGCUCCACCAUGAGU | 116 | − | + |
| miR5018 | | CCAAU | | | |
| ath- | MIMAT0032766 | AGGUGCUGAGUGUGCUAGU | 117 | − | + |
| miR8168 | | GC | | | |
| ath- | MIMAT0004316 | ACAAAAUCCGUCUUUGAAG | 118 | − | + |
| miR866- | | A | | | |
| 3p | | | | | |
| ath- | MIMAT0032769 | UUGCUUAAAGAUUUUCUAU | 119 | − | + |
| miR8170- | | GU | | | |
| 3p | | | | | |
| ath- | MIMAT0000939 | CUGAAGUGUUUGGGGGGAC | 120 | − | + |
| miR395b | | UC | | | |
| ath- | MIMAT0000940 | CUGAAGUGUUUGGGGGGAC | 121 | − | + |
| miR395c | | UC | | | |
| ath- | MIMAT0003939 | UUCUUCGUGAAUAUCUGGC | 122 | | + |
| miR780.2 | | AU | | | |
| ath- | MIMAT0001018 | UAAGCUGCCAGCAUGAUCU | 123 | − | + |
| miR167c- | | UG | | | |
| 5p | | | | | |
| ath- | MIMAT0031905 | AUCAUGCUAUCUCUUUGGA | 124 | − | + |
| miR393a- | | UU | | | |
| 3p | | | | | |
| ath- | MIMAT0000943 | CUGAAGUGUUUGGGGGGAC | 125 | − | + |
| miR395f | | UC | | | |

In vitro studies, as described in U.S. Non-Provisional application Ser. No. 19/270,704, further indicated that plant-derived EVs may influence the activity of mammalian recipient cells. For example, exposure of human dermal fibroblasts to EVs derived from Aloe vera was associated with changes in markers related to extracellular matrix production, including collagen (FIG. 5A and FIG. 5B), elastin, and hyaluronic acid (FIG. 6A, FIG. 6B, and FIG. 6C). Additional studies suggested that such vesicles may modulate signaling pathways associated with angiogenesis and inflammation, including effects on VEGF-related signaling (FIG. 7A, FIG. 7B, and FIG. 7C) and cytokine production in immune cells (FIG. 8).

Additionally experimental work, as described in U.S. Non-Provisional application Ser. No. 19/270,704, explored the interaction of EVs with hair-follicle-associated cells, including dermal papillae cells. Cell proliferation studies in dermal papillae cells showed that test media containing heat shocked human adipose stromal stem cell exosomes and test media containing heat-shocked Aloe-derived exosomes promoted cell proliferation greater than test media with no exosomes (FIG. 9).

Gene expression analyses indicated that treatment with plant-derived EVs may be associated with modulation of genes involved in signaling, growth, and inflammatory pathways. Human follicle dermal papilla cells (HFDPCs) are a group of mesenchymal-derived cells at the base of the hair follicle that regulate hair follicle growth through the expression and secretion of specific biomarkers. Gene expression was assessed using a qPCR-based gene expression panel containing 52 target genes and 4 endogenous control genes. It was found that the cargo of Aloe-derived EVs derived from heat shocked plants modulated gene expression of IL-6 in HFDPCs 24 hours after treatment. Moreover, it was found that the cargo of Aloe-derived EVs derived from heat shocked plants modulated gene expression of CORIN, LEP, IL1B, IL-6, SRD5A2, BMP4, TGFB1, IGF1, HEY1 in HFDPCs 72 hours after treatment.

Antioxidant properties of EV-containing preparations were also evaluated, suggesting that such vesicles may exhibit measurable antioxidant activity. 2,2′-azino-bis-(3-ethylbenzothiazoline-6-sulfonic) acid (ABTS) and oxygen radical absorbance capacity (ORAC) assays performed on different EV samples demonstrated that Aloe-derived EVs and human ASC-derived exosomes have antioxidant capacity (FIG. 10A and FIG. 10B).

As described in U.S. Non-Provisional application Ser. No. 19/270,704, compositions comprising plant-derived EVs were evaluated in clinical and cosmetic contexts, including topical administration for hair and scalp-related conditions. These studies suggested that EV-containing compositions may be well-tolerated and may be associated with improvements for certain conditions. Rapid hair regrowth was shown in subjects with hair loss from various causes including salon coloring treatments, wherein a female subject with hair loss from salon coloring treatments every two weeks was treated with a composition comprising Aloe-derived exosomes in combination with niacinamide, copper tripeptide, biotin, saw palmetto, panthenol, and other ingredients and showed significant hair regrowth after 5 weeks of treatment. Additionally, a female subject with hair loss due to androgenetic alopecia was treated with a similar composition and showed significant hair regrowth 8 weeks after treatment. Similarly, male subjects with post COVID-19 hair loss and alopecia areata were treated with a composition comprising Aloe-derived exosomes in combination with niacinamide, copper tripeptide, biotin, saw palmetto, panthenol, and other ingredients and showed significant hair regrowth 15 weeks and 12 weeks, respectively, after treatment.

Despite these advances, there remains a need in the art for improved methods of producing EVs having defined and tunable molecular cargos, as well as compositions incorporating such vesicles for therapeutic and cosmetic use. In particular, there is a need for systems that enable the generation and use of EVs derived from multiple biological sources, including plant-derived vesicles and mammalian cell-derived vesicles, such as those obtained from adipose-derived stem cells. Such vesicles may provide complementary or synergistic biological effects due to differences in their molecular cargo, including proteins, lipids, and nucleic acids.

The present disclosure describes compositions and methods that combine, or otherwise leverage, distinct EV populations, as well as expanded applications of such vesicles for skin treatment, hair regeneration, wound healing, inflammatory conditions, and mucosal or epithelial tissue health.

### SUMMARY OF THE INVENTION

According to one aspect, the present disclosure provides a composition comprising a purified population of extracellular vesicles (EVs) isolated from one or more biological samples, wherein the size of the EVs is about 50 nm to about 200 nm, inclusive, wherein the purified population of EVs comprise a tuned cargo comprising:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p; and
(ii) a signature of one or more proteins selected from the group consisting of BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, WNT5A, wherein the tuned cargo of the EVs is produced by exposure of the one or more biological samples to one or more stress conditions that cause the biological sample to modulate the EV cargo composition.

In some embodiments, the one or more biological samples comprise a biological sample derived from a tissue, a biological sample derived from cells maintained in culture, a biological sample derived from plants, or a combination thereof.

In some embodiments, the biological sample derived from the tissue comprises tissue explants maintained ex vivo.

In some embodiments, the one or more biological samples comprise a biological sample derived from adipose-derived stem cells, a biological sample derived from Aloe vera plants, or a combination thereof.

In some embodiments, the adipose-derived stem cells are cultured in a bioreactor.

In some embodiments, the adipose-derived stem cells retain mesenchymal stromal cell (MSC) characteristics comprising: (i) adherence to plastic in standard culture conditions; (ii) a surface marker phenotype comprising CD105+, CD73+, CD90+, CD45−, CD34−, CD14− or CD11b−, CD79α− or CD19−, and HLA-DR-; and (iii) a capacity to differentiate into bone, adipose, and cartilage cells.

In some embodiments, the one or more stress conditions comprise hypoxic stress, metabolic stress, mechanical stress, osmotic stress, chemical stress, heat stress, or a combination thereof.

In some embodiments, the tuned cargo of the purified population of extracellular vesicles is produced by exposure of the one or more biological samples to heat stress, wherein the heat stress comprises exposing the one or more biological samples to temperatures between about 30 °C and about 50 °C, inclusive for a period of time between about 10 minutes and about 6 hours, inclusive.

In some embodiments, the heat stress further comprises subsequently incubating the one or more biological samples at a temperature of about 36 °C-38 °C, inclusive, for about 24 hours-72 hours, inclusive.

In some embodiments, the heat stress further comprises subsequently incubating the one or more biological samples at a temperature of about 18 °C-25 °C, inclusive, for about 1 hours-36 hours, inclusive.

In some embodiments, the tuned cargo of the purified population of extracellular vesicles modulates one or more biological activities of a recipient cell, wherein the recipient cell is a human cell.

In some embodiments, the biological activities comprise: (i) modulation of gene expression; (ii) modulation of collagen production; (iii) modulation of cytokine production; (iv) modulation of cell proliferation; (v) modulation of extracellular matrix remodeling; or (vi) modulation of oxidative stress.

In some embodiments, the composition: (i) is a nutraceutical composition comprising a dietary amount of the purified population of extracellular vesicles comprising the tuned cargo; (ii) is a cosmetic composition comprising a cosmetic amount of the purified population of extracellular vesicles comprising the tuned cargo and a cosmetically acceptable carrier; or (iii) is a cosmeceutical composition comprising a cosmeceutical amount of the purified population of extracellular vesicles comprising the tuned cargo and a cosmeceutically acceptable carrier; or (iv) is a pharmaceutical composition comprising a therapeutic amount of the purified population of extracellular vesicles comprising the tuned cargo and a pharmaceutically acceptable carrier.

According to another aspect, the present disclosure provides a method for improving appearance of human skin comprising: (i) obtaining a biological sample comprising a population of extracellular vesicles (EVs) comprising a cargo composition; (ii) modulating the cargo composition by exposing the biological sample comprising the cargo composition to one or more stress conditions to form a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo, and (v) applying the composition topically to human skin, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof; and
(ii) a signature of one or more proteins selected from the group consisting BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, WNT5A.

In some embodiments, the one or more biological samples comprise a biological sample derived from a tissue, a biological sample derived from cells maintained in culture, a biological sample derived from plants, or a combination thereof.

In some embodiments, the biological sample derived from a tissue comprises tissue explants maintained ex vivo.

In some embodiments, the one or more biological samples comprise a biological sample derived from adipose-derived stem cells, a biological sample derived from Aloe vera plants, or a combination thereof.

In some embodiments, the adipose-derived stem cells are cultured in a bioreactor.

In some embodiments, the adipose-derived stem cells retain mesenchymal stromal cell (MSC) characteristics comprising: (i) adherence to plastic in standard culture conditions; (ii) a surface marker phenotype comprising CD105+, CD73+, CD90+, CD45−, CD34−, CD14− or CD11b−, CD79α− or CD19−, and HLA-DR-; and (iii) a capacity to differentiate into bone, adipose, and cartilage cells.

In some embodiments, the one or more stress conditions comprise hypoxic stress, metabolic stress, mechanical stress, osmotic stress, chemical stress, heat stress, or a combination thereof.

In some embodiments, the tuned cargo of the purified population of extracellular vesicles is produced by exposure of the one or more biological samples to heat stress, wherein the heat stress comprises exposing the one or more biological samples to temperatures between about 30 °C to about 50 °C, inclusive for a period of time between 10 minutes to about 6 hours, inclusive.

In some embodiments, the heat stress further comprises subsequently incubating the one or more biological samples at a temperature of about 36 °C-38 °C, inclusive, for about 24 hours-72 hours, inclusive.

In some embodiments, the heat stress further comprises subsequently incubating the one or more biological samples at a temperature of about 18 °C-25 °C, inclusive, for about 1 hours-36 hours, inclusive.

In some embodiments, administering the composition comprising purified population of extracellular vesicles comprising the tuned cargo improves one or more parameters of skin appearance.

In some embodiments, the one or more parameters of skin appearance comprise: (i) reducing the appearance of wrinkles; (ii) reducing skin roughness; (iii) reducing wrinkle depth volume; (iv) reducing wrinkle depth value; (v) increasing skin firmness; (vi) increasing skin luminosity; or (vii) improving one or more symptoms associated with acne.

According to another aspect, the present disclosure provides a method for treating a skin condition, wherein the skin condition is eczema comprising: (i) obtaining a biological sample comprising a population of extracellular vesicles (EVs) comprising a cargo composition; (ii) modulating the cargo composition by exposing the biological sample comprising the cargo composition to one or more stress conditions to form a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo, and (v) applying the composition topically to a region of the skin affected by eczema, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof, and
(ii) a signature of one or more proteins selected from the group consisting BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, or WNT5A.

In some embodiments, the one or more biological samples comprise a biological sample derived from a tissue, a biological sample derived from cells maintained in culture, a biological sample derived from plants, or a combination thereof.

In some embodiments, the biological sample derived from a tissue comprises tissue explants maintained ex vivo.

In some embodiments, the one or more biological samples comprise a biological sample derived from adipose-derived stem cells, a biological sample derived from Aloe vera plants, or a combination thereof.

In some embodiments, the adipose-derived stem cells are cultured in a bioreactor.

In some embodiments, the adipose-derived stem cells retain mesenchymal stromal cell (MSC) characteristics comprising: (i) adherence to plastic in standard culture conditions; (ii) a surface marker phenotype comprising CD105+, CD73+, CD90+, CD45−, CD34−, CD14− or CD11b−, CD79α− or CD19−, and HLA-DR−; and (iii) a capacity to differentiate into bone, adipose, and cartilage cells.

In some embodiments, the one or more stress conditions comprise hypoxic stress, metabolic stress, mechanical stress, osmotic stress, chemical stress, heat stress, or a combination thereof.

In some embodiments, the tuned cargo of the purified population of extracellular vesicles is produced by exposure of the one or more biological samples to heat stress, wherein the heat stress comprises exposing the one or more biological samples to temperatures between about 30 °C to about 50 °C, inclusive for a period of time between 10 minutes to about 6 hours, inclusive.

In some embodiments, the heat stress further comprises subsequently incubating the one or more biological samples at a temperature of about 36 °C-38 °C, inclusive, for about 24 hours-72 hours, inclusive.

In some embodiments, administering the composition comprising purified population of extracellular vesicles comprising the tuned cargo improves one or more symptoms associated with eczema.

In some embodiments, wherein the one or more symptoms associated with eczema comprise: (i) inflammation; (ii) redness; (iii) scaling; (iv) dryness; (v) irritation; or (vi) itchiness.

According to another aspect, the present disclosure provides a method for promoting wound healing or skin repair comprising: (i) obtaining a biological sample comprising a population of extracellular vesicles (EVs) comprising a cargo composition; (ii) modulating the cargo composition by exposing the biological sample comprising the cargo composition to one or more stress conditions to form a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo, and (v) applying the composition topically to a wound, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof, and
(ii) a signature of one or more proteins selected from the group consisting BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, WNT5A.

In some embodiments, the one or more biological samples comprise a biological sample derived from a tissue, a biological sample derived from cells maintained in culture, a biological sample derived from plants, or a combination thereof.

In some embodiments, the biological sample derived from a tissue comprises tissue explants maintained ex vivo.

In some embodiments, the one or more biological samples comprise a biological sample derived from adipose-derived stem cells, a biological sample derived from Aloe vera plants, or a combination thereof.

In some embodiments, the adipose-derived stem cells are cultured in a bioreactor.

In some embodiments, the adipose-derived stem cells retain mesenchymal stromal cell (MSC) characteristics comprising: (i) adherence to plastic in standard culture conditions; (ii) a surface marker phenotype comprising CD105+, CD73+, CD90+, CD45−, CD34−, CD14− or CD11b−, CD79α− or CD19−, and HLA-DR−; and (iii) a capacity to differentiate into bone, adipose, and cartilage cells.

In some embodiments, the one or more stress conditions comprise hypoxic stress, metabolic stress, mechanical stress, osmotic stress, chemical stress, heat stress, or a combination thereof.

In some embodiments, the tuned cargo of the purified population of extracellular vesicles is produced by exposure of the one or more biological samples to heat stress, wherein the heat stress comprises exposing the one or more biological samples to temperatures between about 30 °C to about 50 °C, inclusive for a period of time between 10 minutes to about 6 hours, inclusive.

In some embodiments, the heat stress further comprises subsequently incubating the one or more biological samples at a temperature of about 36 °C-38 °C, inclusive, for about 24 hours-72 hours, inclusive.

In some embodiments, the heat stress further comprises subsequently incubating the one or more biological samples at a temperature of about 18 °C-25 °C, inclusive, for about 1 hours-36 hours, inclusive.

In some embodiments, administering the composition comprising purified population of extracellular vesicles comprising the tuned cargo modulates one or more parameters associated with wound healing or skin repair.

In some embodiments, the one or more parameters associated with wound healing comprise: (i) increasing collagen production; (ii) increasing cell proliferation; (iii) promoting angiogenesis; (iv) promoting immune regulation and reducing inflammation; or (v) promoting tissue regeneration and wound closure.

In some embodiments, administering the composition comprising purified population of extracellular vesicles comprising the tuned cargo improves one or more symptoms associated with gum recession.

According to another aspect, the present disclosure provides a method for promoting hair health comprising: (i) obtaining a biological sample comprising a population of extracellular vesicles (EVs) comprising a cargo composition; (ii) modulating the cargo composition by exposing the biological sample comprising the cargo composition to one or more stress conditions to form a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo; and (v) applying the composition topically to hair, scalp, eyebrows, eyelashes, or a combination thereof, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof, and
(ii) a signature of one or more proteins selected from the group consisting BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, or WNT5A.

In some embodiments, the one or more biological samples comprise a biological sample derived from a tissue, a biological sample derived from cells maintained in culture, a biological sample derived from plants, or a combination thereof.

In some embodiments, the biological sample derived from a tissue comprises tissue explants maintained ex vivo.

In some embodiments, the one or more biological samples comprise a biological sample derived from adipose-derived stem cells, a biological sample derived from Aloe vera plants, or a combination thereof.

In some embodiments, the adipose-derived stem cells are cultured in a bioreactor.

In some embodiments, the adipose-derived stem cells retain mesenchymal stromal cell (MSC) characteristics comprising: (i) adherence to plastic in standard culture conditions; (ii) a surface marker phenotype comprising CD105+, CD73+, CD90+, CD45−, CD34−, CD14− or CD11b−, CD79α− or CD19−, and HLA-DR−; and (iii) capacity to differentiate into bone, adipose, and cartilage cells.

In some embodiments, the one or more stress conditions comprise hypoxic stress, metabolic stress, mechanical stress, osmotic stress, chemical stress, heat stress, or a combination thereof.

In some embodiments, the tuned cargo of the purified population of extracellular vesicles is produced by exposure of the one or more biological samples to heat stress, wherein the heat stress comprises exposing the one or more biological samples to temperatures between about 30 °C to about 50 °C, inclusive for a period of time between 10 minutes to about 6 hours, inclusive.

In some embodiments, the heat stress further comprises subsequently incubating the one or more biological samples at a temperature of about 36 °C-38 °C, inclusive, for about 24 hours-72 hours, inclusive.

In some embodiments, the heat stress further comprises subsequently incubating the one or more biological samples at a temperature of about 18 °C-25 °C, inclusive, for about 1 hours-36 hours, inclusive.

In some embodiments, administering the composition comprising purified population of extracellular vesicles comprising the tuned cargo modulates one or more parameters associated with hair health.

In some embodiments, the one or more parameters associated with hair health comprise: (i) decreasing hair loss; (ii) increasing hair density; (iii) increasing appearance of hair thickness; (iv) improving scalp health; (v) improving hair shine; or (vi) improving hair volume.

According to another aspect, the present disclosure provides a method for promoting vaginal health comprising: (i) obtaining a biological sample comprising a population of extracellular vesicles (EVs) comprising a cargo composition; (ii) modulating the cargo composition by exposing the biological sample comprising the cargo composition to one or more stress conditions to form a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo, and (v) applying the composition topically to vaginal skin, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof, and
(ii) a signature of one or more proteins selected from the group consisting BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, or WNT5A.

In some embodiments, the one or more biological samples comprise a biological sample derived from a tissue, a biological sample derived from cells maintained in culture, a biological sample derived from plants, or a combination thereof.

In some embodiments, the biological sample derived from a tissue comprises tissue explants maintained ex vivo.

In some embodiments, the one or more biological samples comprise a biological sample derived from adipose-derived stem cells, a biological sample derived from Aloe vera plants, or a combination thereof.

In some embodiments, the adipose-derived stem cells are cultured in a bioreactor.

In some embodiments, the adipose-derived stem cells retain mesenchymal stromal cell (MSC) characteristics comprising: (i) adherence to plastic in standard culture conditions; (ii) a surface marker phenotype comprising CD105+, CD73+, CD90+, CD45−, CD34−, CD14− or CD11b−, CD79α− or CD19−, and HLA-DR−; and (iii) a capacity to differentiate into bone, adipose, and cartilage cells.

In some embodiments, the one or more stress conditions comprise hypoxic stress, metabolic stress, mechanical stress, osmotic stress, chemical stress, heat stress, or a combination thereof.

In some embodiments, the tuned cargo of the purified population of extracellular vesicles is produced by exposure of the one or more biological samples to heat stress, wherein the heat stress comprises exposing the one or more biological samples to temperatures between about 30 °C to about 50 °C, inclusive for a period of time between 10 minutes to about 6 hours, inclusive.

In some embodiments, the heat stress further comprises subsequently incubating the one or more biological samples at a temperature of about 36 °C-38 °C, inclusive, for about 24 hours-72 hours, inclusive.

In some embodiments, the heat stress further comprises subsequently incubating the one or more biological samples at a temperature of about 18 °C-25 °C, inclusive, for about 1 hours-36 hours, inclusive.

In some embodiments, administering the composition comprising purified population of extracellular vesicles comprising the tuned cargo modulates one or more parameters associated with vaginal health.

In some embodiments, the one or more parameters associated with vaginal health comprise: (i) tissue regeneration and renewal; (ii) increasing collagen production; (iii) extracellular matrix remodeling; (iv) promoting angiogenesis; (v) reducing inflammation; (vi) reducing vaginal dryness; or (vii) increasing vaginal sensitization.

### BRIEF DESCRIPTION OF THE DRAWINGS

FIG. 1 is a schematic diagram of skin anatomy.

FIG. 2 is a schematic of cell types in the layers of the skin from the skin surface to the dermis, including the stratum corneum, the stratum lucidum, stratum granulosum, stratum germinativum, and stratum basale. Each contains living cells with specialized functions.

FIG. 3 is a schematic of the molecular structure of the mammalian circadian clock. The circadian clock consists of three core feedback loops. The newly described feedback loop has also been depicted. Loop 1: basic helix loop helix ARNT like 1 (BMAL1) gene dimerizes with Clock Circadian Regulator (CLOCK) gene or Neuronal PAS Domain Protein 2 (NPAS2) gene. These dimers bind to the promoter region E-box elements (5′-CACGTG-3′), triggering Period 1-3 (PER1-3), crystalline (CRY), retinoic acid-related orphan receptor (ROR), nuclear receptor subfamily 1, group D, member 1(NR1D1), and transcription factor albumin D site-binding protein (DBP) gene expression. In mammals, DBP rhythmically activates transcription of various genes through a DNA cis-element, D-box. PER and CRY dimerize on reaching a critical concentration, inhibiting their own expression, causing oscillation of the expression of these proteins. Loop 2: The transcription of BMAL1, CLOCK, and NFIL3 (a rhythmically expressed transcription factor) is triggered when ROR binds to the RORE element 5′-(A/G)GGTCA-3′ in their promoter region. This binding of ROR to RORE is inhibited by NR1D1. Loop 3: PER transcription is initiated when the D box element (5′-TTATG(T/C)AA-3′) in its promoter region is bound to by DBP (Loop1). This binding is negatively regulated by NFIL3 (Loop 2). DEC loop: The DEC protein that gives this loop its name, is responsible for its own oscillatory expression by inhibiting the binding of BMAL1: CLOCK to E-box elements (5′-CACGTG-3′) in its promoter. (taken from Salazar, A. and von Hagen, J., Intl J. Molec. Sci. (2023) 24: 5635).

FIG. 4 is a bar graph showing HSP70 gene expression in purified Aloe-derived exosomes before treatment and after heat stress treatment compared to the whole plant and a pre-treatment control.

FIG. 5A and FIG. 5B are bar graphs depicting collagen 1 production in human dermal fibroblasts. FIG. 5A is a bar graph of collagen 1 (ng/mL) in human dermal fibroblasts against a media control; treatment samples (Aloe 1, Aloe 2, and adipose-derived stem cells (ASC)); and a TGFβ positive control showing that Aloe-derived purified exosomes can induce collagen I synthesis in human dermal fibroblasts. FIG. 5B is a bar graph showing percent change of collagen in human dermal fibroblasts treated as in FIG. 5A.

FIG. 6A, FIG. 6B, and FIG. 6C show bar graphs of fold change in production of elastin (left), hyaluronic acid (middle) and collagen 1 (right) in human skin dermal fibroblasts treated with Aloe-derived exosomes FIG. 6A Prep 1 (1×10E8, 3.33×10E7, 1,11×10E7), and Prep 2 (1.00×10E8, 3.33E7, 1.11×10E7), FIG. 6B Prep 3 (1×10E8, 3.33×10E7, 1,11×10E7), and Prep 4 (1.00×10E8, 3.33×10E7, 1.11×10E7), and FIG. 6C Prep 5 (1×10E8, 3.33×10E7, 1,11×10E7) compared to a media control.

FIG. 7A, FIG. 7B, and FIG. 7C show bar graphs of VEGF-A production in human dermal fibroblasts. FIG. 7A is a bar graph of VEGF-A concentration (ng/mL) in human dermal fibroblasts treated with 5% FBS (positive control), Aloe-derived exosomes (1×10E9) Aloe Exosomes (1×10E8), and Aloe-derived exosomes (1×10E7). FIG. 7B shows VEGFA concentration fold change; vs. 5% FBS positive control, Aloe-derived exosomes (1×10E9) Aloe-derived exosomes (1×10E8), and Aloe-derived exosomes (1×10E7). FIG. 7C shows VEGFA concentration (ng/mL) versus IL-8 (1000 ng/mL). IL-8 (100 mg/mL), IL-8 (10 ng/mL), IL-8 (1 ng/mL), IL-8 (0.1 ng/mL), and 10% FBS positive control.

FIG. 8 shows average cytokine production (pg/mL interferon a2 (INFa2) in PBMCs in vitro in response to bacterial exposure. Treatment groups from left to right are: media control; adipose-derived stem cells (ASC)-derived exosomes; Aloe exosomes.

FIG. 9 shows a bar graph depicting the effect of heat-shocked (HS) human ASC-derived exosomes and HS Aloe-derived exosomes on cell proliferation of hair dermal papillae, compared to media controls (control media, test media).

FIG. 10A and FIG. 10B are bar graphs showing in vitro antioxidant activity of Aloe-derived exosomes and human ASC-derived exosomes. FIG. 10A shows antioxidant activity of Aloe-derived exosomes and human ASC-derived exosomes by 2,2′-azino-bis-(3-ethylbenzothiazoline-6-sulfonic) acid (ABTS). FIG. 10B shows antioxidant activity of Aloe-derived exosomes and human ASC-derived exosomes by oxygen radical absorbance capacity (ORAC).

FIG. 11 is a schematic of the signaling cascade induced by cytokines that signal via the Jak-STAT signaling pathway [Taken from Morris, R. et al. Protein Science (2018) 27: 1984-2009]. Cytokine binds to a specific receptor and allows transactivation of the associated Janus Kinases (JAKs). Activated JAKs then phosphorylate tyrosines on the intracellular domains of the receptor, which recruit the Signal Transducers and Activators of Transcription (STAT) transcription factors. STATs are translocatd into the nucleus and upregulate the transcription of cytokine-responsive genes. Suppressors of cytokine signaling (SOCS) proteins are direct targets of STAT and act as negative-feedback inhibitors to switch off the signaling cascade.

FIG. 12, taken from Su, J. et al. Front. Immunol. (2021) 11: 593564, is a schematic representation of leptin and leptin receptor signal transduction pathways and functions. Stimulation of the leptin receptor by leptin can activate Janus (JAK2) kinase, resulting in tyrosine phosphorylation of the receptor and downstream proteins, including Signal transducer and Activator of Transcription 3 (STAT3), a member of the signal transducers and activators of transcription (STAT) transcription factor family, which functions together with the janus kinases (JAK) in JAK-STAT signaling networks commonly activated by cytokines, protein tyrosine phosphatase 2 (SHP-2), an SH2 domain-containing protein tyrosine phosphatase, Insulin Receptor Substrate 2 (IRS2, an adaptor molecule that plays a fundamental role in insulin and IL-4, IL-7 and IL-9 signaling), and phosphoinositide 3-kinase (PI3K) that play a role in regulating transcription of genes essential for energy intake and lipid metabolism. ACC (acetyl-CoA carboxylase); AMPK (adenosine monophosphate kinase); CPTI (carnitine palmitoyltransferase 1); ERK (extracellular signaling-regulated kinase); FAS (fatty acid synthase); SCD1 (stearoyl-coenzyme A desaturace 1, enzyme in fatty acid metabolism); TG (triglycerides); PL (phospholipids); CE (cholesterol esters).

FIG. 13 is a schematic of the mitogen activated protein kinase (MAPK) signaling pathways [taken from Soares-Silva, M. et al. Front. Microbiol. (2016) 7: 183]. The major MAPK pathways involved in inflammatory diseases are extracellular regulating kinase (ERK), p38 MAPK, and c-Jun NH2-terminal kinase (JNK). All three MAPK pathways may be activated by TGF-β, and signaling through these cascades can further regulate the expression of Smad proteins and mediate Smad-independent TGF-βresponses. Smaroteins are key signal transducers for the TGF-βsuperfamily. These three MAPK pathways are all involved in TGF-β-induced fibrosis [He, W. and Dai, C. Curr. Pathobiol. Rep. (2015) 3: 183-92, citing Tsou, P S et al. Am. J. Physiol. Cell Physiol. (22014) 307: C2-13; Kamato, D. et al. Cell Signal (2013) 25: 2017-24; Pannu, J. et al. J. Biol. Chem. (2007) 282: 10405-13; Yu, L. et al. J. Biol. Chem. (2002) EMBO J. 21: 3749-59]. Upstream kinases include TGFβ-activated kinase-1 (TAK1) and apoptosis signal-regulating kinase-1 (ASK1). Downstream of p38 MAPK is MAPK activated protein kinase 2 (MAPKAPK2 or MK2). TGF-0 can signal in a noncanonical manner via the MAPK family.

FIG. 14 is a schematic of the PI3K/Akt/mTOR pathway (Taken from Porta, et al. Front. Oncol. (2014) 4: art. 64). Activation of growth factor receptor protein tyrosine kinases results in autophosphorylation on tyrosine residues. PI3K is then recruited to the membrane by directly binding to phosphotyrosine consensus residues of growth factor receptors or adaptors through one of the two SH2 domains in the adaptor subunit. This leads to allosteric activation of the catalytic (CAT) subunit of PI3K. PI3K activation leads to the production of the second messenger phosphatidylinositol-4,4-bisphosphate (PI3,4,5-P3) from the substrate phosphatidylinositol-4,4-bisphosphate (PI-4,5-P2). PI3,4,5-P3 then recruits a subset of signaling proteins with pleckstrin homology (PH) domains to the membrane, including protein serine/threonine kinase-3′-phosphoinositide-dependent kinase I (PDK1) and Akt/protein kinase B (PKB) [Id., citing Fruman, D A et al., Phosphoinositide kinases. Annu. Rev. Biochem. (1998) 67: 481-507, Fresno-Vara, J A, et al., PI3K/Akt signaling pathway and cancer. Cancer Treat. Rev. (2004) 30: 193-204]. Akt (also known as protein kinase B) is activated in response to stimulation of tyrosine kinase receptors, such as platelet-derived growth factor (PDGF), insulin-like growth factor, and nerve growth factor. Stimulation of Akt has been shown to be dependent on phosphatidylinositol 3-kinase (PI3-kinase) activity. Mammalian target of rapamycin (mTOR) is an atypical serine/threonine kinase that is present in two distinct complexes. The first, mTOR complex 1 (mTORC1), is composed of mTOR, regulatory protein associated with mTOR (Raptor), GOL (also known as mammalian lethal with Sec13 protein 8 or mLST8), and DEP domain containing m-TOR Interacting Protein (DEPTOR) and is inhibited by rapamycin. It is a master growth regulator that senses and integrates diverse nutritional and environmental cues, including growth factors, energy levels, cellular stress, and amino acids. It couples these signals to the promotion of cellular growth by phosphorylating substrates that potentiate anabolic processes such as mRNA translation and lipid synthesis or limit catabolic processes such as autophagy. The small GTPase Rheb, in its GTP-bound state, is a necessary and potent stimulator of mTORC1 kinase activity, which is negatively regulated by its GTPase-activating protein (GAP), the tuberous sclerosis heterodimer TSC1/2. TSC1 and TSC2 are the tumor-suppressor genes mutated in the tumor syndrome TSC (tuberous sclerosis complex). Their gene products form a complex (the TSC1-TSC2 (hamartin-tuberin) complex), which, through its GAP activity towards the small G-protein Rheb (Ras homologue enriched in brain), is a critical negative regulator of mTORC1. Most upstream inputs are funneled through Akt and TSC1/2 to regulate the nucleotide-loading state of Rheb. The second mTOR complex (mTORC2) is sensitive to growth factors, not nutrients, and is associated with rapamycin-insensitivity mTORC2 is thought to modulate growth factor signaling by phosphorylating the C-terminal hydrophobic motif of some cAMP-dependent, cGMP-dependent and protein kinase C (AGC) kinases such as Akt and SGK [Jhanwar-Uniyal, M. et al. Adv. Biol. Regul. (2015) 57: 64-74].

FIG. 15 is a schematic of the IGF-1 signaling pathway [taken from Iams, W T and Lovly, CM, Clin. Cancer Res. (2015) 21 (19): 42770-77]. Insulin-like growth factors (IGFs) bind specifically to the IGF1 receptor on the cell surface of targeted tissues. Ligand binding to the a subunit of the receptor leads to a conformational change in the β subunit, resulting in the activation of receptor tyrosine kinase activity. Activated receptor phosphorylates several substrates, including insulin receptor substrates (IRSs) and Src homology collagen (SHC). Phosphotyrosine residues in these substrates are recognized by certain Src homology 2 (SH2) domain-containing signaling molecules. These include an 85 kDa regulatory subunit (p85) of phosphatidylinositol 3-kinase (PI 3-kinase), growth factor receptor-bound 2 (GRB2) and SH2-containing protein tyrosine phosphatase 2 (SHP2/Syp), which lead to the activation of downstream signaling pathways, PI 3-kinase pathway and Ras-mitogen-activated protein kinase (MAP kinase) pathway.

FIG. 16 is a scanning electron microscopy (SEM) image of adipose-derived stem cell (ASC)-derived exosomes.

FIG. 17 is a bar graph of quantified western blot data that shows the amount of HSP70 protein relative to actin protein in two separate preparations of exosomes: secreted by adipose-derived stem cells (ASCs) cultured at 37 °C without a heat shock step (native); and secreted by ASCs subjected to a 2 hour heat shock step at 43 °C.

FIG. 18 shows a western blot quantification assessing HSP70 levels in adipose-derived stem cell (ASC)-derived exosome preparations, wherein the ASCs were sourced from two different sources (source 1 and source 2): (1) ASC source 1, native exosomes (no heat shocking); (2) ASC source 1, tuned (heat shocked) exosomes; (3) ASC source 2, native exosomes (no engineering); and (4) ASC source 2, tuned (heat shocked) exosomes, indicating that preparation (2) had the greatest effect on HSP70 expression. HSP70 protein expression was normalized to β-actin as a loading control.

FIG. 19 shows the relative changes in HSP70 mRNA transcript levels in exosomes isolated from Aloe vera plants for each of the following: non-heat shocked, native Aloe-derived exosomes (NATIVE), Aloe-derived exosomes isolated after heat shock at 35 °C for 2 hours (METHOD A), Aloe-derived exosomes isolated after heat shock at 45 °C for 3 hours (METHOD B), Aloe-derived exosomes isolated 24 hours after a 3-hour heat shock at 45 °C (METHOD C). Values shown are Relative Quantification values determined by the Ct method (Fold Change) relative to the NATIVE exosome values. Statistical significance compared to the NATIVE sample was determined using student's t-test and a p-value <0.05, significance is denoted by a“*”.

FIG. 20A and FIG. 20B show that treatment with the tuned adipose-derived stem cell (ASC)-derived exosomes isolated from the heat shocked cells increased collagen I production of both human periodontal ligament fibroblasts (PDLFs) and dermal fibroblasts (DFs), as compared to the isolated ASC-derived exosomes prepared from cells that were not subjected to a heat shock step. FIG. 20A is a graph showing the amount of collagen I production in dermal fibroblasts after a 48 hour incubation with a media control (“no treatment”), various growth factors (IGF, PDGF, TGF beta), or exosomes secreted from cells cultured with (“engineered exosome”) or without a heat shock (“exosome”) step. Values shown on the Y axis are ng/ml of collagen. FIG. 20B is a graph showing the amount of collagen I production in periodontal ligament fibroblasts after a 48 hour incubation with a media control (“no treatment”), various growth factors (IGF, PDGF, TGF beta), or exosomes secreted from cells cultured with (“engineered exosome”) or without a heat shock (“exosome”) step. Values shown on the Y axis are ng/ml of collagen.

FIG. 21 shows collagen I production, as measured by a homogenous time resolved fluorescence (HTRF) ELISA, in human dermal fibroblast cells (10,000 cells/well) treated for 48 hours with native Aloe-derived exosomes (Native), Aloe-derived exosomes isolated after heat shock at 35 °C for 2 hours (EXO A), Aloe-derived exosomes isolated after heat shock at 45 °C for 3 hours (EXO B), Aloe-derived exosomes isolated 24 hours after a 3-hour heat shock at 45 °C (EXO C), a media only control (Media), and a control treated with 20 ng/ml TGFβ-1 (Control). The highest collagen I production was measured for exosomes obtained using manufacturing methodology EXO A.

FIG. 22 shows collagen I production, as measured by an ELISA assay, in primary human fibroblasts (PHFs) that were treated for 48 hours with serum-free conditioned medium, TGF beta-1, increasing concentrations of native ASC-derived exosomes (1 pg, 3 pg, and 10 pg), and tuned ASC-derived exosomes (“engineered exosomes”) at 5 pg. The conditioned medium was removed, clarified by centrifugation, and diluted for ELISA analysis. The highest collagen production was measured for engineered exosomes.

FIG. 23 shows IL-6 secretion in human peripheral blood mononuclear cells (PBMCs) treated with Lipopolysaccharide (LPS) (1 pg/ml) and exosomes isolated according to the following manufacturing methods: native Aloe-derived exosomes (no heat shocking), or tuned Aloe-derived exosomes manufactured by Method 1 (exosomes isolated after exposure to 35 °C for 2 hours), Method 2 (exosomes isolated after exposure to 35 °C for 2 hours, followed by 45 °C for 3 hours), Method 3 (exosomes isolated after exposure to 35 °C for 2 hours, followed by 45 °C for 3 hours, followed by 18-25 °C for 6 hours), or Method 4 (exosomes isolated after exposure to 35 °C for 2 hours, followed by 45 °C for 3 hours, followed by 18-25 °C for 24 hours). Dexamethosone was included as a control. Method 1 promoted the greatest level of IL-6 secretion.

FIG. 24 shows TNF-α secretion in human peripheral blood mononuclear cells (PBMCs) treated with Lipopolysaccharide (LPS) (1 pg/ml) and exosomes isolated according to the following manufacturing methods: native Aloe-derived exosomes (no engineering), or engineered Aloe-derived exosomes manufactured by Method 1 (exosomes isolated after exposure to 35 °C for 2 hours), Method 2 (exosomes isolated after exposure to 35 °C for 2 hours, followed by 45 °C for 3 hours), Method 3 (exosomes isolated after exposure to 35 °C for 2 hours, followed by 45 °C for 3 hours, followed by 18-25 °C for 6 hours), or Method 4 (exosomes isolated after exposure to 35 °C for 2 hours, followed by 45 °C for 3 hours, followed by 18-25 °C for 24 hours). Dexamethosone was included as a control. Method 1 promoted the greatest level of TNF-α secretion.

FIG. 25A, FIG. 25B, and FIG. 25C show photographs of a subject with visible crow's feet wrinkles in the periorbital region before and after treatment with a moisturizer comprising tuned adipose-derived stem cell (ASC)-derived exosomes. FIG. 25A shows the periorbital skin before treatment. FIG. 25B shows the periorbital skin during the course of treatment. FIG. 25C shows periorbital skin after treatment, indicating improvement over the course of the treatment.

FIG. 26A, FIG. 26B, and FIG. 26C show photographs of a subject with visible acne on the face before and after treatment with a moisturizer comprising tuned adipose-derived stem cell (ASC)-derived exosomes. FIG. 26A shows the face before treatment. FIG. 26B shows the face during the course of treatment. FIG. 26C shows face after treatment, indicating improvement over the course of the treatment.

FIG. 27A and FIG. 27B show photographs of a subject with visible acne on the face before and after treatment with a moisturizer comprising tuned adipose-derived stem cell (ASC)-derived and tuned Aloe-derived exosomes. FIG. 27A shows the face before treatment. FIG. 27B shows the face following 7 days of treatment, indicating improvement over the course of the treatment.

FIG. 28A and FIG. 28B show photographs of a subject with improved skin hydration and overall skin firmness on the upper arm following treatment with the tuned adipose-derived stem cell (ASC)-derived exosome-containing topical formulation. FIG. 28A shows the upper arm before treatment. FIG. 28B shows the upper arm following 2 weeks of treatment, indicating improvement over the course of the treatment.

FIG. 29 is a graph depicting the size of tuned adipose-derived stem cell (ASC)-derived exosomes (eXo3), as measured by nanoparticle tracking analysis (NTA), indicating a median diameter of about 124.6 nm and a mode diameter of 90.5 nm.

FIG. 30A and FIG. 30B are transmission electron microscopy (TEM) images depicting tuned adipose-derived stem cell (ASC)-derived (eXo3) exosome morphology. FIG. 30A shows a zoomed-out TEM image, scale bar indicating 200 nm. FIG. 30B shows a magnified TEM image, indicating that the tuned ASC-derived exosomes are about 100 nm in diameter, scale bar indicating 100 nm.

FIG. 31 shows a bar graph demonstrating an immunofluorescence quantification showing expression of exosome surface protein markers detected by antibodies on the tuned adipose-derived stem cell (ASC)-derived exosomes (eXo3), including CD81, CD63, and CD9.

FIG. 32 shows bar graphs of fold induction (y-axis) vs treatment (x-axis) depicting levels of the inflammatory cytokines IL-6, IL-8, and monocyte chemoattractant protein-1 (MCP-1) following treatment of human fibroblasts with tuned ASC-derived exosomes (eXo3), P. gingivalis (HKPG), and a no treatment control for 12 hours.

FIG. 33A, FIG. 33B, FIG. 33C, and FIG. 33D show photographs of a left leg of a subject with visible eczema before and after twice-daily treatment with a topical lotion emulsion formulation comprising tuned adipose-derived stem cell (ASC)-derived exosomes indicating improvement over the course of the treatment. FIG. 33A shows the leg before treatment. FIG. 33B shows a magnified image of the leg before treatment indicating visible eczema. FIG. 33C shows the leg 21 days after treatment. FIG. 33D shows the leg 56 days after treatment.

FIG. 34A and FIG. 34B show photographs of a subject with visible eczema on the back of the knee before and after twice-daily treatment with a topical lotion emulsion formulation comprising tuned adipose-derived stem cell (ASC)-derived exosomes. FIG. 34A shows the back of the knee before treatment. FIG. 34B shows the back of the knee 18 days after treatment, indicating improvement over the course of the treatment.

FIG. 35A, FIG. 35B, FIG. 35C, FIG. 35D, and FIG. 35E shows results of a Radius® plate cell migration assay using human primary skin epithelial cells, seeded at 100,000 cells/well, in which a biocompatible Radius™ hydrogel creates a circular area across which seeded cells may migrate following gel removal. FIG. 35A shows that at T=48 hours, serum free media (negative control) showed minimal migration. FIG. 35B shows that 1% FBS media exhibits slightly more migration. FIG. 35C shows that 10% FBS media showed full closure of the gap. FIG. 35D shows the addition of 25×10E6 tuned ASC-derived exosomes (eXo3) to the 1% FBS media, which revealed slightly better gap closure compared to the 1% FBS media alone (FIG. 35B). FIG. 35E shows that 10×10E7 tuned ASC-derived exosomes (eXo3) elicited complete gap closure when added to the 1% FBS media.

FIG. 36A, FIG. 36B, FIG. 36C, FIG. 36D, and FIG. 36E show enhanced vascular networks with tuned ASC-derived exosome (eXo3) treatment of human umbilical vein endothelial cells (HUVECs) when compared to a serum free treatment control, a 10% FBS treatment control, and a platelet-derived growth factor treatment control. FIG. 36A shows HUVECs seeded in Matrigel and treated with a serum free non-heat shocked exosome treatment control. FIG. 36B shows HUVECs seeded in Matrigel and treated with a 10% FBS treatment control. FIG. 36C shows HUVECs seeded in Matrigel and treated with control (non-heat shocked) EVs. FIG. 36D shows HUVECs seeded in Matrigel and treated with tuned ASC-derived exosomes (eXo3). FIG. 36E shows HUVECs seeded in Matrigel and treated with a platelet-derived growth factor (PDGF) treatment control.

FIG. 37A, FIG. 37B, FIG. 37C, and FIG. 37D shows wound healing using a rat model. FIG. 36A shows four excision wounds made on the animal at day 0. FIG. 37B shows day 14 of treatment with either vehicle or tuned adipose-derived stem cell (ASC)-derived exosomes (eXo3). Bottom left circles represent vehicle control. FIG. 37C shows day 14 of treatment with either vehicle or lyophilized tuned ASC-derived exosomes (lyophilized eXo3). Bottom left circles represent vehicle control. FIG. 37D is a plot of % wound closure (y-axis) versus time in days over a 20 day period. ASC-derived exosome treatment shows enhanced wound closure. ANOVA p<0.05.

FIG. 38A, FIG. 38B, and FIG. 38C show tissue sections stained with Verhoeff-Van Gieson (EVG stain) to identify changes in collagen fibers in rat excisional wound sites 8 days after injury. The tissue is stained with a regressive hematoxylin, consisting of ferric chloride and iodine. The differentiating is accomplished by using excess mordant (ferric chloride) to break the tissue-mordant dye complex. FIG. 38A shows collagen production in a non-wounded rat. FIG. 38B shows collagen production in a vehicle-treated wound. FIG. 38C shows increased collagen production in a wound treated with tuned adipose-derived stem cell (ASC)-derived exosomes (eXo3) (arrows).

FIG. 39A, FIG. 39B, and FIG. 39C shows results of staining rat excisional wound sites 8 days after injury to detect Ki-67, a nuclear marker for proliferating cells. Ki67 is present during all active phases of the cell cycle (G1, S, G2, and M) but is absent in resting cells (G0). FIG. 39A shows Ki-67 staining in a non-wounded rat. FIG. 39B shows Ki-67 staining in a vehicle-treated wound. FIG. 39C shows increased Ki-67 staining in a wound treated with tuned ASC-derived exosomes (eXo3).

FIG. 40A, FIG. 40B, FIG. 40C, and FIG. 40D show photographs of human volunteer single case study observations of a foot of a patient with chronic diabetic ulcers after topical application of tuned ASC-derived exosomes indicating improvement over the course of the treatment. FIG. 40A shows the diabetic ulcers before treatment. FIG. 40B shows the diabetic ulcers 30 days after treatment. FIG. 40C shows the diabetic ulcers 60 days after treatment. FIG. 40D shows the diabetic ulcers 120 days after treatment.

FIG. 41A, FIG. 41B, and FIG. 41C show photographs of post-surgery scars of a subject with a history of keloid formation after topical application of tuned ASC-derived exosomes indicating improvement over the course of the treatment. FIG. 41A shows the scars before treatment. FIG. 41B shows the scars during the treatment with ASC-derived exosomes on day 14. FIG. 41C shows the scars after 21 days of treatment.

FIG. 42A and FIG. 42B show photographs of a lip ulceration of a subject after topical application of tuned adipose-derived stem cell (ASC)-derived exosomes indicating improvement over the course of the treatment. FIG. 42A shows the lip ulceration before treatment. FIG. 42B shows the lip ulceration 16 hours after treatment.

FIG. 43A and FIG. 43B show photographs of a subject's gum recession. FIG. 43A shows the gum recession before treatment with tuned adipose-derived stem cell (ASC)-derived exosomes. FIG. 43B shows the gum recession 7 days after the treatment with tuned ASC-derived exosomes twice daily, indicating improvement over the course of the treatment.

FIG. 44A and FIG. 44B shows an increase in hair density in a male subject after topical application of Hairgenyl® a peptide-rich ingredient from the yeast Pichia minuta, isolated from the flowers of azalea (Silab) indicating improvement over the course of the treatment. FIG. 44A shows the subject on day 0 before application; FIG. 44B shows the subject after 8 months of application.

FIG. 45A and FIG. 45B shows Ki-67 and versican staining of a control hair follicle. Versican is a large ECM proteoglycan that plays important roles in cell-matrix interactions during adhesion, migration and inflammatory responses. FIG. 45A shows versican and Ki-67 staining of a hair follicle treated with an untreated control. FIG. 45B shows enhanced versican and Ki-67 staining of a hair follicle treated with 0.04% Hairgenyl®.

FIG. 46A, FIG. 46B, and FIG. 46C show photographs of a male subject with male pattern hair loss after topical application of tuned adipose-derived stem cell (ASC)-derived exosomes indicating improvement over the course of the treatment with EXO Hair formulation HC-002 and HC-003. FIG. 46A shows the hair before treatment. FIG. 46B shows the hair on day 45 of treatment. FIG. 46C shows the hair 9 weeks after treatment.

FIG. 47A and FIG. 47B show photographs of a subject with male pattern hair loss after topical application of tuned adipose-derived stem cell (ASC)-derived exosomes indicating improvement over the course of the treatment with EXO Hair formulation HC-001 and HC-002. FIG. 47A shows the hair before treatment. FIG. 47B shows the hair 12 weeks after treatment.

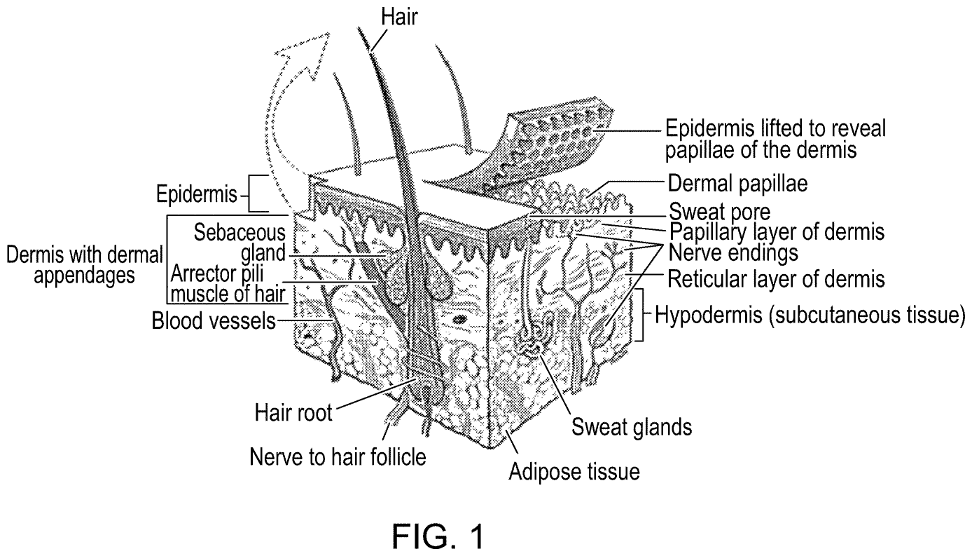

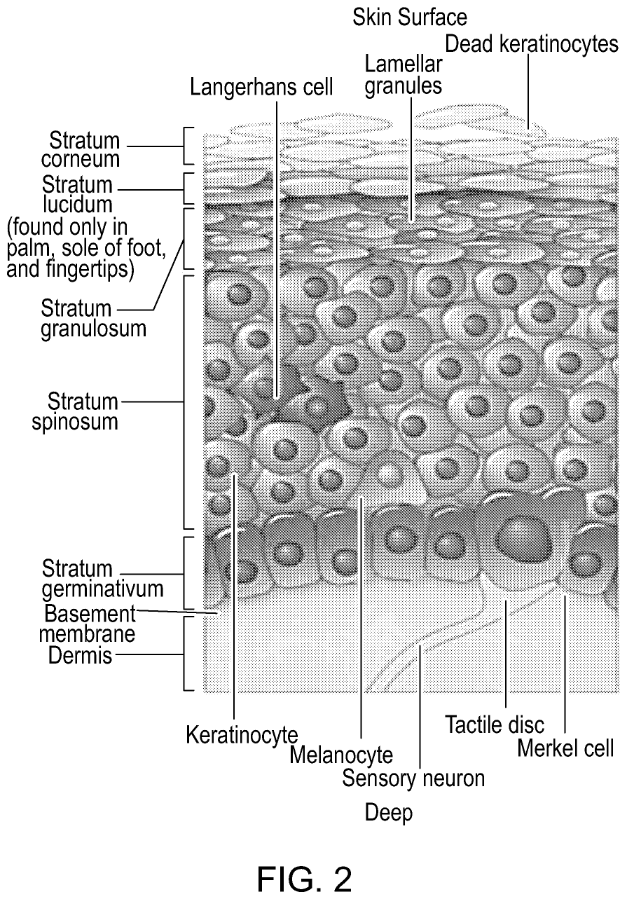

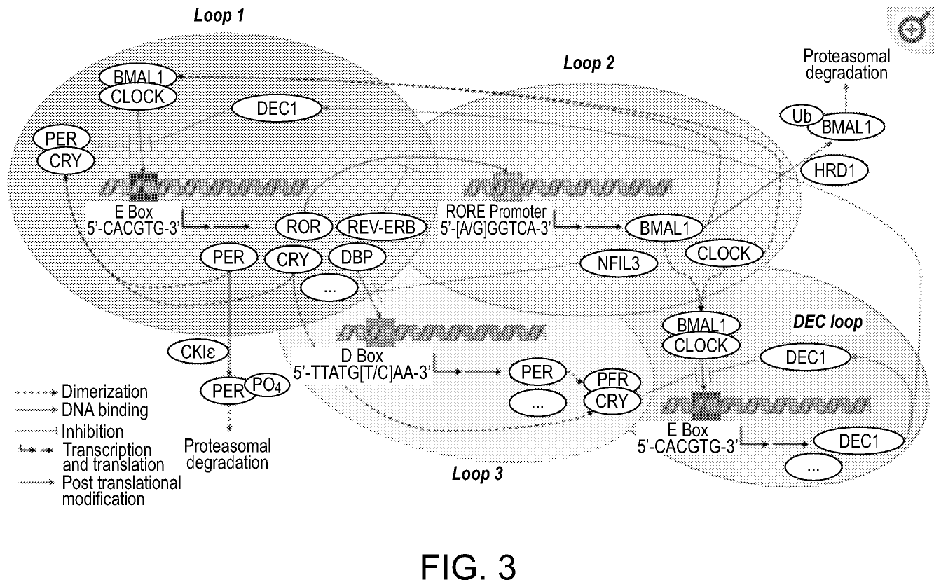

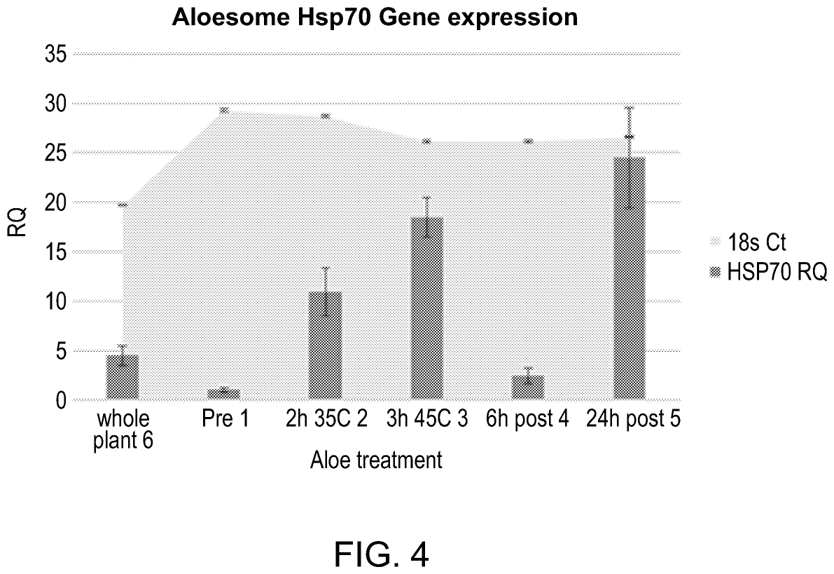

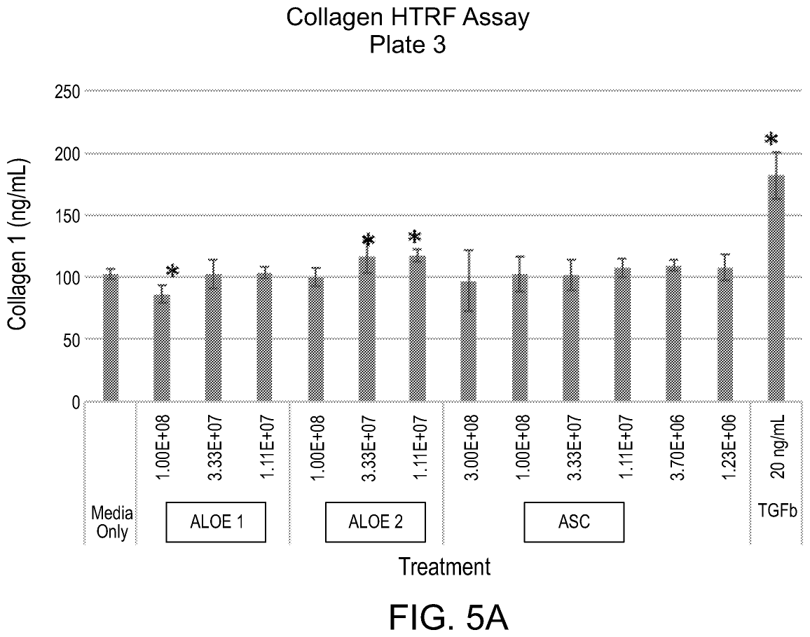

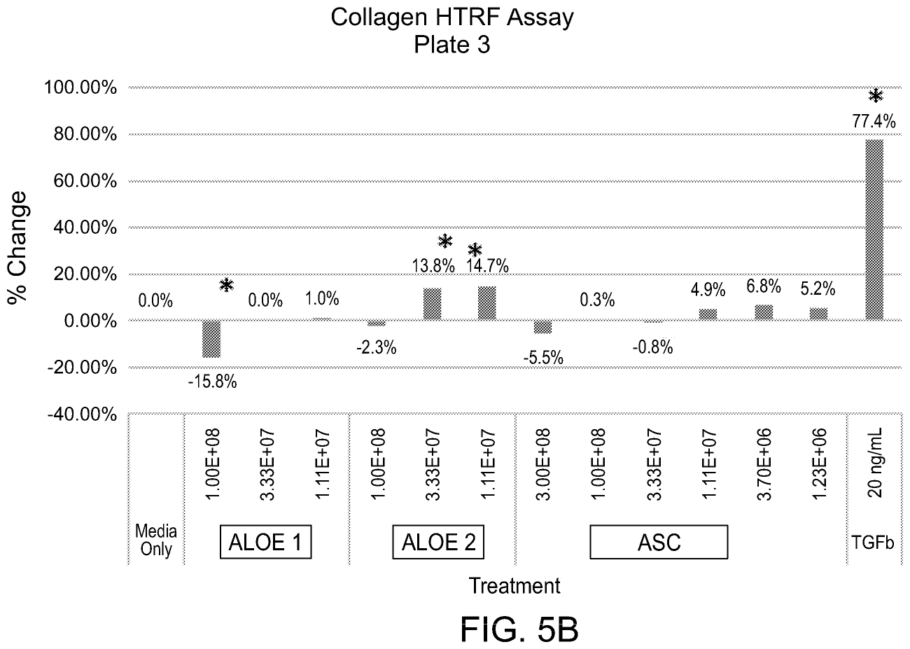

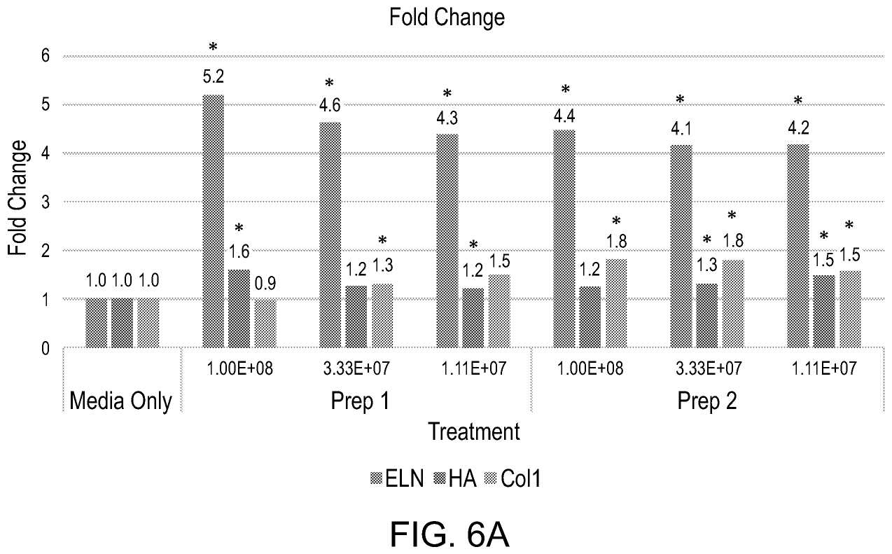

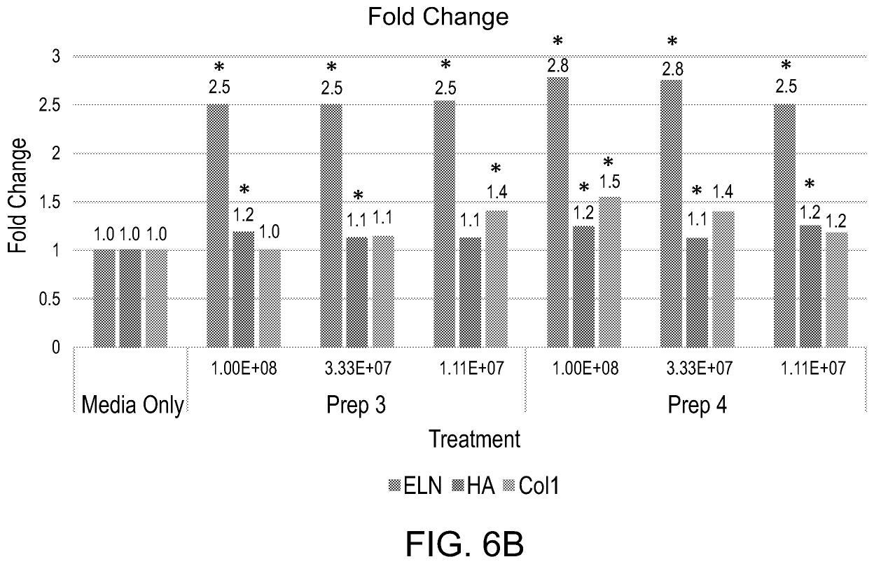

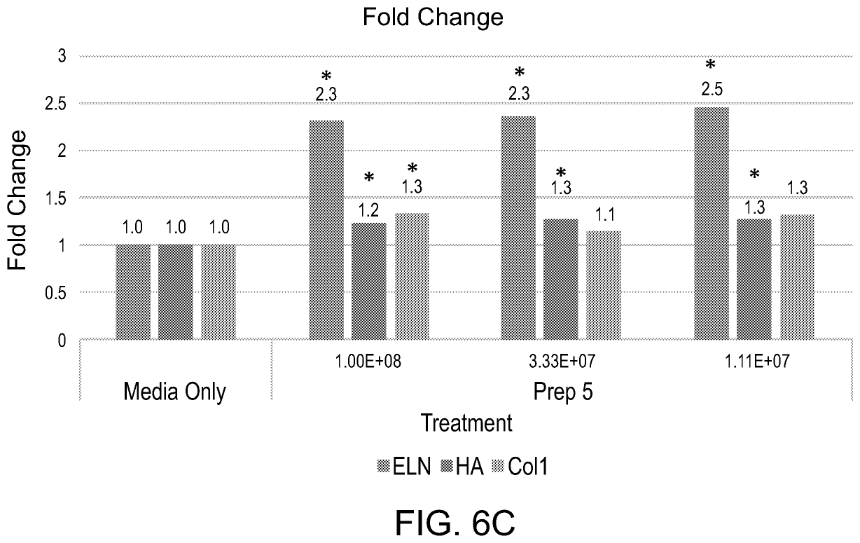

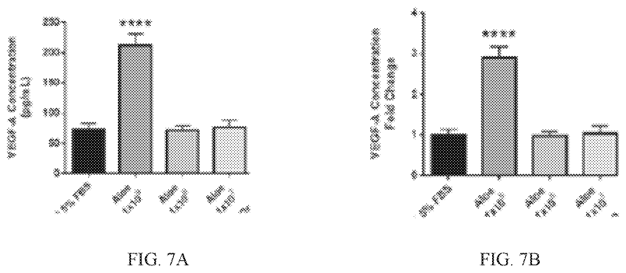

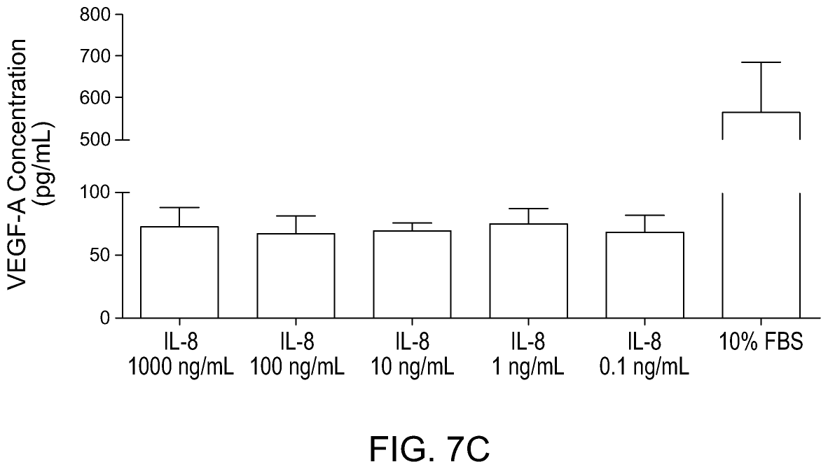

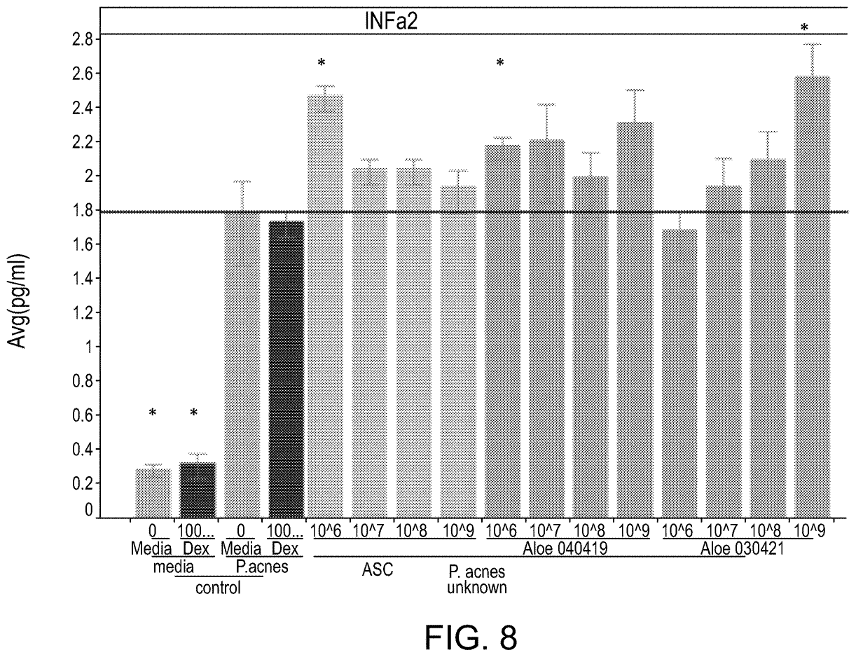

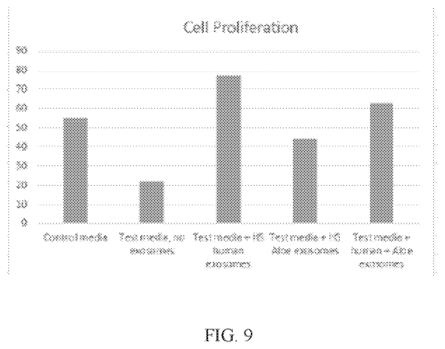

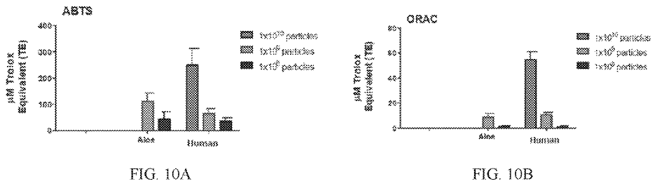

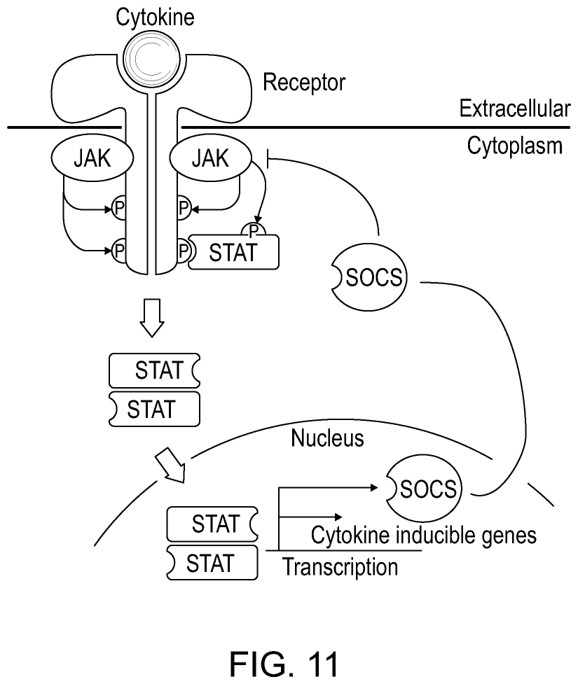

### DETAILED DESCRIPTION OF THE INVENTION

### Definitions

As used herein the term “10E×” is scientific notation meaning 10 raised to the X power. For example, 10E10 means ten raised to the tenth power, or 1×10¹⁰.

As used herein and in the appended claims, the singular forms “a” “an”, and “the” include plural reference unless the context clearly dictates otherwise. Thus, for example, reference to a “peptide” is a reference to one or more peptides and equivalents thereof known to those skilled in the art, and so forth.

The term “about” is used herein to mean within the typical ranges of tolerances in the art. For example, “about” can be understood as about 2 standard deviations from the mean. According to certain embodiments, about means+10%. According to certain embodiments, about means+5%. When about is present before a series of numbers or a range, it is understood that “about” can modify each of the numbers in the series or range.

The term “at least” prior to a number or series of numbers (e.g. “at least two”) is understood to include the number adjacent to the term “at least”, and all subsequent numbers or integers that could logically be included, as clear from context. When at least is present before a series of numbers or a range, it is understood that “at least” can modify each of the numbers in the series or range.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

As used herein, “up to” as in “up to 10” is understood as up to and including 10, i.e., 0, 1, 2, 3, 4, 5, 6, 7, 8, 9, or 10.

The term “acoustic separation”, as used herein refers to the use of acoustofluidic devices to create standing surface acoustic waves (SSAW) that apply contact-free acoustic radiation forces, pushing larger EVs and contaminants toward pressure nodes while smaller particles are removed by fluid flow. This microfluidic approach enables rapid, continuous-flow isolation, with high-frequency (e.g., 40 MHz) waves separating submicrometer particles.

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Full responsiveness of a T cell requires, in addition to receptor engagement, an accessory cell-delivered costimulatory activity, e.g., engagement of CD28 on the T cell by CD80 and/or CD86 on the antigen presenting cell (APC).

The term “active” refers to the ingredient, component or constituent of the compositions of the described invention responsible for the intended cosmetic, cosmeceutical or therapeutic effect.

The term “adapted cell” refers to cells that have undergone structural or functional changes to survive and maintain homeostasis in response to environmental stresses. Types of cellular adaptation include atrophy (i.e., decrease in cell size and number), hypertrophy (i.e., increase in cell size), hyperplasia (i.e., increase in number), metaplasia (i.e., replacement of one cell type with another), dysplasia (i.e., abnormal changes in cell size, shape, or organization, often considered pre-cancerous), and necrosis (i.e., dying cells).

The term “adaptive immunity” as used herein refers to a specific, delayed and longer-lasting response by various types of cells that create long-term immunological memory against a specific antigen. It can be further subdivided into cellular and humoral branches, the former largely mediated by T cells and the latter by B cells. Adaptive immunity further encompasses cell lineage members of the adaptive arm that have effector functions in the innate arm, thereby bridging the gap between the innate and adaptive immune response.

“Administering” when used in conjunction with a cosmetic, cosmeceutical or therapeutic means to give or apply a cosmetic, cosmeceutical or therapeutic directly into or onto a target organ, tissue or cell, or to administer the cosmetic, cosmeceutical or therapeutic to a subject, whereby the cosmetic, cosmeceutical or therapeutic positively impacts the organ, tissue, cell, or subject to which it is targeted. Thus, as used herein, the term “administering”, when used in conjunction with EVs or compositions thereof, can include, but is not limited to, providing EVs into or onto the target organ, tissue or cell; or providing EVs systemically to a subject, whereby the therapeutic reaches the target organ, tissue or cell. “Administering” may be accomplished by oral, parenteral or topical administration or by such methods in combination with other known techniques.

As used herein, the term “affinity capture” refers to a process that isolates and purifies EVs, cells, proteins, nucleic acids, etc. by targeting specific surface molecules (i.e., proteins, lipids) using immobilized ligands (i.e., antibodies, receptors) on a solid phase, such as magnetic beads or chip surfaces.

As used herein, the term “air-lift bioreactor” refers to a vessel configured for culturing cells in a liquid medium so that mixing and circulation of the medium are achieved primarily through the introduction of air or a gas That creates density differences and fluid flow within the vessel. The airlift bioreactor has one or more zones including a riser zone and a downcomer zone that facilitates continuous circulation of cells nutrients and dissolved gasses under controlled conditions.

The term “allele” as used herein refers to any of one or more alternative forms of a given gene. Generally, the alleles of a given gene are concerned with the same trait or characteristic, but the product or function coded for by a particular allele may differ, quantitatively and/or qualitatively, from that coded for by other alleles of that gene. A “wild-type allele” is one which codes for a particular phenotypic characteristic found in the wild type strain of a given organism.

The term “allogeneic” as used herein refers to being genetically different although belonging to or obtained from the same species.

The term “allograft” as used herein refers to a transplant of tissue from an allogeneic donor of the same species.

The term “allograft immunity” as used herein refers to any immune response by the host against a transplanted tissue.

The term “alopecia” is a medical term for the absence or loss of hair including eyelashes, eyebrows, and scalp hair, as a result of illness, functional disorder, or hereditary disposition. Alopecia frequently occurs in patients undergoing treatment for cancer or suffering from other diseases, such as AIDS, where cytotoxic drugs are used.

The term “alopecia adnata” refers to underdevelopment of the eyelashes.

The term “alopecia areata” refers to a common condition of undetermined etiology characterized by circumscribed, nonscarring, usually asymmetrical areas of baldness on the scalp, eyebrows, and beaded portion of the face. Hairy skin anywhere on the body may be affected. It is thought to be an autoimmune disease occurring on areas of the body (most commonly the scalp) where the person's immune system attacks hair follicles, thereby suppressing and arresting hair growth.

The term “amino acid” is used to refer to an organic molecule containing both an amino group and a carboxyl group; those that serve as the building blocks of naturally occurring proteins are alpha amino acids, in which both the amino and carboxyl groups are linked to the same carbon atom. The terms “amino acid residue” or “residue” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids.

The abbreviations used herein for amino acids are those abbreviations which are conventionally used: A=Ala=Alanine; R=Arg=Arginine; N=Asn=Asparagine; D=Asp=Aspartic acid; C=Cys=Cysteine; Q=Gln=Glutamine; E=Glu=Glutamic acid; G=Gly=Glycine; H=His=Histidine; I=Ile=lsoleucine; L=Leu=Leucine; K=Lys=Lysine; M=Met=Methionine; F=Phe=Phenyalanine; P=Pro=Proline; S=Ser=Serine; T=Thr=Threonine; W=Trp=Tryptophan; Y=Tyr=Tyrosine; V=Val=Valine. The amino acids may be L- or D-amino acids. An amino acid may be replaced by a synthetic amino acid which is altered so as to increase the half-life of the peptide or to increase the potency of the peptide, or to increase the bioavailability of the peptide.

The following represent groups of amino acids that are conservative substitutions for one another:
Alanine (A), Serine (S), Threonine (T);
Aspartic Acid (D), Glutamic Acid (E);
Asparagine (N), Glutamine (Q);
Arginine (R), Lysine (K);
Isoleucine (I), Leucine (L), Methionine (M), Valine (V); and
Phenylalanine (F), Tyrosine (Y), Tryptophan (W).

The term “amplicon” as used herein refers to DNA or RNA products of amplification reactions, e.g., a polymerase chain reaction (PCR) product.

The term “anagen effluvium” refers to the hair loss associated with chemotherapeutic agents that cause immediate destruction and release of anagen hair.

The term “androgenic alopecia” refers to a gradual decrease of scalp hair density in adults with transformation of terminal to vellus hairs, which become lost as a result of familial increased susceptibility of hair follicles to androgen secretion following puberty. The most common form of androgenic alopecia is male pattern baldness. The most common form of androgenic alopecia in women is female pattern alopecia, a diffuse partial hair loss in the centroparietal area of the scalp, with preservation of the frontal and temporal hairlines. When it occurs in females, it is associated with other evidence of excessive androgen activity, such as hirsuitism.

The term “anesthetic agents” as used herein refers to agents that resulting in a reduction or loss of sensation.

The term “anti-acne” as used herein refers to agents that alleviate the symptoms of acne. The term “acne” as used herein refers to an inflammatory disease of the sebaceous glands, characterized by comedones and pimples.

The term “angiogenesis” as used herein refers to the formation of new blood vessels from pre-existing vasculature. Angiogenesis is central to a number of physiological conditions, from embryogenesis to wound healing, and is a hallmark of pathological conditions, such as tumorigenesis [Abhinand, C S et al. J. Cell Commun. Signal. (2016) 10 (4): 347-54].

The terms “animal,” “patient,” and “subject” as used herein include, but are not limited to, humans and non-human vertebrates such as wild, domestic and farm animals. According to some embodiments, the terms “animal,” “patient,” and “subject” may refer to humans. According to some embodiments, the terms “animal,” “patient,” and “subject” may refer to non-human mammals.

The term “anionic” as used herein refers to a molecule, substance, solvent, etc. that carries a negative charge.

The term “annexins” as used herein refers to Ca+2-dependent proteins that bind to membrane phospholipids in plants [Laohavisit, A. and Davies, J M. New Phytologist (2011) 189: 40-53]. They have been found to be stimulated by abiotic stress, including light, salinity, heat, cold, drought; oxidative and mechanic stress [Saad, R B et al. Plant Signaling & Behavior (2020) 15 (1): e1699264; Baucher, M. et al. Plant Signaling & Behavior (2012) 7 (4): 524-8]. Annexin expression also has been shown to be upregulated during pathogen attack or symbiotic interaction [Baucher, M. et al. Plant Signaling & Behavior (2012) 7 (4): 524-8, citing de Carvadho, N F, et al. Mol. Plant Microbe Interact. (1998) 11: 504-13; Manthey, K. et al. Mol. Plant Microbe Interact (2004) 17: 1063-77; Vandeputte, O. et al. Mol. Plant Pathol. (2007) 8: 185-94]. The dynamic properties of plant annexins together with their expression patterns suggest that they are involved in cell signaling processes and therefore in plant development al adaptation to environmental changes.

The term “antibiotic agent” as used herein means any of a group of chemical substances having the capacity to inhibit the growth of, or to destroy bacteria, and other microorganisms, used chiefly in the treatment of infectious diseases.

The term “anti-dandruff agents” as used herein refers to agents that reduce, eliminate or prevent a scurf from forming on skin, especially of the scalp, that comes off in small white or grayish scales.

The term “antihistamine agent” as used herein refers to any of various compounds that counteract histamine in the body and that are used for treating allergic reactions (such as hay fever) and cold symptoms.

The term “anti-irritant” as used herein refers to an agent that prevents or reduces soreness, roughness, or inflammation of a bodily part.

The term “anti-oxidant agent” as used herein refers to a substance that inhibits oxidation or reactions promoted by oxygen or peroxides.

The term “anti-protozoal agent” as used herein means any of a group of chemical substances having the capacity to inhibit the growth of or to destroy protozoans used chiefly in the treatment of protozoal diseases.

The term “antipruritic agents” as used herein refers to those substances that reduce, eliminate or prevent itching.

The term “anti-skin atrophy actives” refers to substances effective in replenishing or rejuvenating the epidermal layer by promoting or maintaining the natural process of desquamation.

The term “anti-wrinkle agent” as used herein an agent intended to reduce the appearance of wrinkles in the skin.

The term “antigen” as used herein, is meant to refer to a molecule containing one or more antigenic determinants or epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 7, 8, 9, 10, 11, 12, 13, 14, or 15 amino acids. The term includes polypeptides which include modifications, such as deletions, additions and substitutions (generally conservative in nature) as compared to a native sequence, as long as the protein maintains the ability to elicit an immunological response. These modifications may be deliberate, as through site-directed mutagenesis, or may be accidental, such as through mutations of the host that produces the antigens.

The term “antigen presentation” as used herein, generally refers to the display of antigen on the surface of a cell, e.g., in the form of peptide fragments bound to MHC molecules.

As used herein, the term “antigen presenting cell (APC)” refers to a class of cells capable of displaying on its surface (“presenting”) one or more antigens in the form of peptide-MHC complex recognizable by specific effector cells of the immune system and thereby inducing an effective cellular immune response against the antigen or antigens being presented. Examples of professional APCs are dendritic cells and macrophages, though any cell expressing MHC Class I or II molecules can potentially present peptide antigen. An APC can be an irradiated population of PBMCs. An APC can be an “artificial APC,” meaning a cell that is engineered to present one or more antigens. Before a T cell can recognize a foreign protein, the protein has to be processed inside an antigen presenting cell or target cell so that it can be displayed as peptide-MHC complexes on the cell surface.

As used herein the term “antigen processing” refers to the intracellular degradation of foreign proteins into peptides that can bind to MHC molecules for presentation to T cells.

As used herein, the term “AR” refers to a gene that is involved in the regulation of androgen hormones. As people age, androgens tend to stimulate hair growth in some areas of the body (such as facial hair) and inhibit hair growth in other areas (such as the scalp). AR expression can also downregulate BMP2 and BMP4.

The term “Argonaute 2” or “AGO2” as used herein refers to an RNA binding protein that can shuttle between the cytoplasm and nucleus in a context-dependent fashion [Sharma, N R et al. J. Biol. Chem. (2016) 291: 2302-9] and is a key effector of RNA-silencing pathways. It is a major component of the RNA-induced silencing complex (RISC).

The term “astringents” are generally protein precipitants that have such low cell penetrability that the action essentially is limited to the cell surface and interstitial spaces. Astringents are locally applied. The astringent action is accompanied by contraction and wrinkling of the tissue and by blanching. Astringents are used therapeutically to arrest hemorrhage by coagulating the blood, to promote healing, to toughen the skin or to decrease sweating. The principal components of astringents are salts of aluminum, zinc, manganese, iron or bismuth.

As used herein, the term “AXIN2” refers to a well-established Wnt/β-catenin target gene that is expressed in hair follicle stem cells and in the outer bulge throughout telogen and anagen. Upregulated expression of axis inhibition protein 2 (Axin2) prevents the onset of catagen, with the result being significantly longer and thicker hair.

As used herein, the term “base pair” or “bp” refers to a unit consisting of two nucleobases bound to each other by hydrogen bonds. Generally, the size of an organism's genome is measured in base pairs because DNA is typically double stranded. However, some viruses have single-stranded DNA or RNA genomes.

The term “binding” and its other grammatical forms as used herein means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “biomarker” (or “biosignature”) as used herein refers to a peptide, a protein, a nucleic acid, an antibody, a gene, a metabolite, or any other substance used as an indicator of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

As used herein, the term “biopsy” refers to any tissue or cellular sample taken from a subject, which can reflect a biological state of the subject. A biopsy sample can include any tissue or material derived from a living or dead subject. A biopsy sample can comprise a nucleic acid (e.g., DNA or RNA) or a fragment thereof.

As used herein, the term “bioreactor” refers to a controlled system or device configured to support the growth, maintenance, and/or expansion of living cells or tissues under defined environmental conditions. Such conditions include controlled temperature, pH, oxygen concentration, nutrient supply, and mechanical or fluidic forces (e.g., agitation, perfusion, or shear stress).

The terms “BMP2” and “BMP4” are used herein to refer to bone morphogenetic proteins (BMPs), which are a group of peptide growth factors that belong to the TGF-beta (TGF-β) superfamily. They regulate gene expression via regulating the recruitment of transcription factors. Traditionally, the inhibition of BMPs has been shown to promote anagen in hair follicles, where the downregulation of BMPs is associated with increased activation of Wnt signaling. Accumulation of BMP-inhibitory signals can initiate fresh hair growth. Downregulation of BMP2 was seen in cultured human hairs with prolonged anagen phases and in the accelerated regrowth of hair in mice. BMP4 is expressed in the hair shaft precursors (precortical matrix) and in the dermal papilla. BMP4 regulates hair follicle cycling and is important for hair cycle morphogenesis. BMP2 and BMP4 are expressed in arrested hair follicles. Downregulation of BMP4 leads to anagen development in hair follicles. Upregulation of BMP6 in dermal papilla cells leads to decreased cell migration. BMP4 and BMP6 also arrest the hair follicle during the telogen phase (resting stage). BMP6 and Wnt10b competitively regulate the telogen-anagen transition. When BMP6 is upregulated and Wnt10b is downregulated, cells do not process to anagen and remain in telogen; when BMP6 is downregulated and Wnt10b is upregulated, cells transition to anagen normally and hair growth occurs.

As used herein, the term “BrdU assay” or “5-bromo-2′-deoxyuridine assay” refers to a thymidine analog that incorporates into newly synthesized DNA of actively proliferating cells and is quantified in a method using anti-BrdU antibodies.

As used herein, the term “bridge amplification” refers to an amplification technique involving hybridization of adapter-containing nucleic acid molecules to surface-bound primers, followed by amplification to generate clusters of clonally amplified molecules on a surface.

As used herein, the term “carboxyfluorescein assay” or “CFSE assay” refers to an assay that relies on the ability of CFSE to covalently label long-lived intracellular molecules with the highly fluorescent dye, carboxyfluorescein, following each cell division, the equal distribution of these fluorescent molecules to progeny cells results in a halving of the fluorescence of daughter cells.

The term “cargo” as used herein refers to a load or that which is conveyed. With respect to exosomes and/or extracellular vesicles, the term cargo refers to a substance encapsulated in the exosome and or extracellular vesicle. The compound or substance can be, e.g., a nucleic acid (e.g., nucleotides, DNA, RNA), a polypeptide, a lipid, a protein, or a metabolite, or any other substance that can be encapsulated in an exosome and/or an extracellular vesicle.

The term “cargo profile” as used herein refers to measurements of cargo components that characterize a population of extracellular vesicles.

The term “carrier” as used herein describes a material that does not cause significant irritation to a mammal and does not abrogate the biological activity and properties of the actives of the composition. Carriers must be of sufficiently high purity and of sufficiently low toxicity to render them suitable for administration to the mammal being treated. The carrier can be inert, or it can possess pharmaceutical benefits, cosmetic benefits or both.

The term “cationic” as used herein refers to a molecule, substance, solvent, etc. that carries a positive charge.

The term “caustic agents” as used herein refers to substances capable of destroying or eating away epithelial tissue by chemical action. Caustic agents can be used to remove dead skin cells. For example, beta-hydroxy acids, naturally derived acids with a strong kerolytic effect, are useful for problem skin, acne or peeling.

The term “cell of origin” refers to the specific parent cell that produces, packages, and secretes the microvesicle into the extracellular environment.

The term “cell surface marker” as used herein refers to an antigenic determinant or epitope found on the surface of a specific type of cell. Cell surface markers can facilitate the characterization of a cell type, its identification, and eventually its isolation. Cell sorting techniques are based on cellular biomarkers where a cell surface marker(s) may be used for either positive selection or negative selection, i.e., for inclusion or exclusion, from a cell or EV population.

The term “chaotropic agent” as used herein refers to a substance that destabilizes the structure of macromolecules (i.e., proteins, nucleic acids, lipids) by disrupting hydrogen bonding and weakening hydrophobic interactions, which increases molecular disorder.

The term “clonal” as used herein refers to of or relating to a clone.

The term “clone” as used herein refers to a population of cells or organisms derived asexually from a single progenitor. A clone is generally assumed to be genetically homogeneous.

“Cluster of Differentiation” or “cluster of designation” (CD) molecules are utilized in cell sorting using various methods, including flow cytometry. Cell populations usually are defined using a “+” or a “−” symbol to indicate whether a certain cell fraction expresses or lacks a particular CD molecule.

The term “CD9” as used herein refers to a member of the tetraspanin protein family whose crystal structure shows a reversed cone-like molecular shape, which generates membrane curvature in the crystalline lipid layers. (Umeda, R. et al. Nature Communic. (2020) 11: article 1606).

The term “CD63” as used herein refers to a member of the tetraspanin protein family, the C-terminal domain of which interacts with several subunits of adaptor protein (AP) complexes, linking the traffic of this tetraspanin to clathrin-dependent pathways (Andreu, Z. & Yanez-Mo, M., citing Rous, B A et al. Mol. Biol. Cell (2002) 13 (3): 1071-1082). Among intracellular interacting proteins, CD63 was shown to directly bind to syntenin-1, a double PDZ domain-containing protein (Id., citing Latysheva, N. et al. Mol. Cell Biol. (2006) 26 (20): 7707-7718). A major role in exosome biogenesis has been reported for Syntenin-1 (Id., citing Baietti, M F et al. Nat. Cell Biol. (2012) 14 (7): 677-685).

The term “CD81” as used herein refers to a member of the tetraspanin protein family whose crystal structure shows a reversed teepee-like arrangement of the four transmembrane I helices, which create a central pocket in the intramembranous region that appears to bind cholesterol in the central cavity. (Zimmerman, B. et al. Cell (2016) 167: 1041-1051). During development, CD81 regulates the trafficking of CD19, an essential co-stimulatory molecule of lymphoid B cells and a well-characterized CD81 partner, along the secretory pathway. (Shoham, T. et al. J. Imunol. (2003) 171: 4062-4072). CD9 and CD81 have been shown to regulate several cell-cell fusion processes. (Charrin, S. et al. J. Cell Science (2014) 127: 3641-3648).

The term “CD82” as used herein refers to a member of the tetraspanin protein family that has been implicated in the regulation of protein sorting into EVs and in antigen presentation by antigen presenting cells. (Andreu, Z. and Yanez-Mo, M. Front. Immunol. (2014) doi.org/10.3389/fimmu.2014.00442).

The term “compatible” as used herein means that the components of a composition are capable of being combined with each other in a manner such that there is no interaction that would substantially reduce the efficacy of the composition under ordinary use conditions.

The term “complementary DNA” or “cDNA” as used herein refers to synthetic DNA that has been transcribed from a specific mRNA through a reaction using the enzyme reverse transcriptase. While DNA is composed of both coding and non-coding sequences, cDNA contains only coding sequences.

As used herein, the term “cDNA library” refers to a collection of DNA sequences generated from RNA sequences. The cDNA library can represent the RNA present in the original sample from which the RNA was extracted. Accordingly, in some aspects, a cDNA library can represent all or a part of a transcriptome of a given cell or population of cells including messenger RNA (mRNA), ribosomal RNA (rRNA), transfer RNA (tRNA) and other noncoding RNA (ncRNA) produced in one cell or a population of cells.

The term “condition” as used herein includes a variety of conditions related to skin or mucosal membranes. This term is meant to include disorders or diseases, the promotion of healthy epithelium; dry skin; and inflammation caused by any underlying mechanism or disorder.

The term “conditioned medium” (or plural, media), as used herein refers to spent culture medium harvested from cultured cells containing metabolites, growth factors, RNA and proteins released into the medium by the cultured cells.

The term “contact” and its various grammatical forms as used herein refers to a state or condition of touching or of immediate or local proximity.

The term “cosmeceutical” as used herein refers to a topical preparation sold as a cosmetic that has performance characteristics that affect the skin positively beyond the time of its application.

The term “cosmetic composition” as used herein refers to a composition that is intended to be rubbed, poured, sprinkled, or sprayed on, introduced into, or otherwise applied to a subject or any part thereof for cleansing, beautifying, promoting attractiveness, or altering the appearance, or an article intended for use as a component of any such article, except that such term does not include soap.

The terms “cosmetic signature”, cosmeceutical signature” and “therapeutic signature” as used herein respectively refer to a specific and complex combination of biomarkers that reflect a biological state that leads to a specific cosmetic, cosmeceutical or therapeutic effect.

The term “cosmetically acceptable carrier” as used herein refers to a substantially non-toxic carrier, conventionally useable for the topical administration of cosmetics, with which compounds will remain stable and bioavailable.

The terms “cosmetic amount”, cosmeceutical amount” or “pharmaceutical amount” as used herein refer to the amount of any of the compositions of the invention that result in a beneficial effect following administration to a subject. The cosmetic, cosmeceutical, or pharmaceutical effect can improve physical appearance and aesthetics, treating, a condition, disease or disorder, or any other beneficial effect. The concentration of the active(s) is selected so as to exert its cosmetic, cosmeceutical or pharmaceutical effect, but low enough to avoid significant side effects within the scope and sound judgment of the skilled artisan. The effective amount of the composition may vary with the particular epithelial tissue being treated, the age and physical condition of the biological subject being treated, the severity of the condition, the duration of the treatment, the nature of concurrent therapy, the specific compound(s), composition or other active ingredient(s) employed, the particular carrier utilized, and like factors. A skilled artisan can determine an effective amount of the inventive compositions by determining the unit dose.

The term “cross-dressing” as used herein refers to a third pathway for cross-presentation. In cross-dressing, dendritic cells acquire preformed MHC class I molecules in complex with antigens from other cells by the process of trogocytotis (meaning the transfer of cell membrane patches or individual proteins between cells [Yewdell, J W and Dolan, BP, Nature (2011) 471 (7340): 581-582, citing Joly, E. and Hudrisier, D. Nature Immunol. (2003) 4: 815; Herrera O B et al. J. Imunol. (2004) 173: 4828-4837] or through gap junctions. This allows antigen presentation by acceptor dendritic cells to occur immediately, without any processing. Cross-dressing is used to activate memory T cells, but not naïve T cells, in response to viral infection [Id., citing Wakins, L M and Bevan, MJ. Nature (2011) 471: 629-632].

The term “cross-presentation” as used herein refers to a process by which proteins taken up by dendritic cells from the extracellular milieu can give rise to peptides presented by MHC class I molecules. It enables antigens from extracellular sources to be presented by MHC class I molecules and to activate CD8 T cells.

The term “cross-priming” as used herein refers to activation of CD8 T cells by dendritic cells in which the antigenic peptide presented by MHC class I molecules is derived from an exogenous protein (i.e., by cross-presentation), rather than produced within the dendritic cells directly (compare direct presentation).

The term “culture medium” (or plural, media), as used herein refers to a substance containing nutrients in which cells or tissues are cultivated for controlled growth.

The term “cytokine” as used herein refers to small soluble protein substances secreted by cells, which have a variety of effects on other cells. Cytokines mediate many important physiological functions, including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (TNF)-related molecules, including TNFα and lymphotoxin; immunoglobulin super-family members, including interleukin 1 (IL-1); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term damage-associated molecular patterns” or “DAMPS” as used herein refers to molecules released by stressed or dying cells that bind to pattern recognition molecules (PRMs) and induce inflammation.

As used herein, the term “DCN” refers to a small, leucine-rich proteoglycan found in the extracellular matrix of the dermal cell layer. Proteoglycans have been suggested to play an important role in hair biology. Studies have shown that DCN is highly expressed in hair follicle epithelial cells and dermal papilla cells during the anagen phase. Other studies were able to confirm that the addition of exogenous DCN accelerated anagen and delayed catagen. In decorin knockdown mice, the telogen-to-anagen transition was delayed in hair follicles while the anagen-to-catagen transition was accelerated.

The term “decrease” or “reduce” and their various grammatical forms is used herein to refer to a diminution, a reduction, an attenuation or abatement of the degree, intensity, extent, size, amount, density or number of occurrences, events or characteristics. As used herein, the term “increase” and its various grammatical forms refers to becoming or making greater in size, amount, intensity, or degree, such as a n increase in the number of occurrences, events or characteristics.

The term “dendritic cells (DC)” as used herein refers to professional antigen presenting cells, which induce naïve T cell activation and effector differentiation [Patente, T A, et al., Frontiers Immunol. (2019) doi.org/10.3389/fimmu.2018.03176]. Human DC are identified by their high expression of major histocompatibility complex (MHC) class II molecules (MHC-II) and of CD11c, both of which are found on other cells, like lymphocytes, monocytes and macrophages [Id., citing Carlens J, et al. J Immunol. (2009) 183:5600-5607; Drutman S B, et al. J Immunol. (2012) 188:3603-3610; Hochweller K, S et al. Eur J Immunol. (2008) 38:2776-2783; Huleatt J W, Lefrangois L. J Immunol. (1995) 154:5684-5693; Rubtsov A V, et al. Blood (2011) 118:1305-15; Probst H C, et al. Clin Exp Immunol. (2005) 141:398-404; Vermaelen K, Pauwels R. Cytometry (2004) 61A:170-177].

The term “density gradient centrifugation” as used herein refers to a laboratory technique that separates components of a sample including cells, organelles, viruses, and biomolecules based on their density. It relies on the principle that, under centrifugal force, particles will migrate through a medium until they reach a point where the surrounding density equals their own. For example, EVs can be separated from contaminants based on their unique buoyancy, rather than size. The technique involves layering a sample onto a gradient medium (e.g., sucrose or iodixanol) of varying densities and centrifuging at high speeds (usually over 100,000 g) for extended periods of time (i.e., 6-18 hours).

The term “DNA” or “deoxyribonucleic acid” as used herein refers to a polynucleotide formed from covalently linked deoxyribonucleotide units.

As used herein, the term “derived from” is meant to encompass any method for receiving, obtaining, or modifying something from a source of origin.

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like.

As used herein, the terms “detecting”, “determining”, and their other grammatical forms, are used to refer to methods performed for the identification or quantification of a biomarker, such as, for example, the presence or level of miRNA, or for the presence or absence of a condition in a biological sample. The amount of biomarker expression or activity detected in the sample can be none or below the level of detection of the assay or method. The term “detectable response” as used herein, is meant to refer to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

As used herein, the term “differential centrifugation” as used herein refers to a laboratory technique in which a stepwise increase in centrifugation speed (typically ranging from 300 g to over 100,000 g) to remove cells, debris, and large vesicles, is followed by high-speed ultracentrifugation to pellet small EVs. This process separates components based on density and size.

The term “differential expression” refers to a statistically significant difference in expression levels of a gene between two samples or populations of samples. The statistically significant difference in expression can refer to increased gene expression or reduced gene expression. The expression levels can differ, for example, by at least a factor of 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or greater. Differential expression also includes expression of genes in a given sample and not in other samples, i.e., the other samples are substantially free of expression in those genes that are expressed in the given sample.

The term “differentiation” as used herein refers to a process of development with an increase in the level of organization or complexity of a cell or tissue, accompanied by a more specialized function.

The term “direct presentation” as used herein refers to a process by which proteins produced within a given cell give rise to peptides presented by MHC class I molecules. This may refer to APCs (such as dendritic cells), or to nonimmune cells that will become the targets of CTLs.

When used to describe the expression of a gene or polynucleotide sequence, the terms “down-regulation”, “disruption”, “inhibition”, “inactivation”, and “silencing” are used interchangeably herein to refer to instances when the transcription of the polynucleotide sequence is reduced or eliminated. This results in the reduction or elimination of RNA transcripts from the polynucleotide sequence, which results in a reduction or elimination of protein expression derived from the polynucleotide sequence (if the gene comprised an ORF). Alternatively, down-regulation can refer to instances where protein translation from transcripts produced by the polynucleotide sequence is reduced or eliminated. Alternatively, still, down-regulation can refer to instances where a protein expressed by the polynucleotide sequence has reduced activity. The reduction in any of the above processes (transcription, translation, protein activity) in a cell can be by about 20%, 30%, 40%, 50%, 60%, 70%, 80%, 90%, 95%, or 100% relative to the transcription, translation, or protein activity of a suitable control cell. Down-regulation can be the result of a targeting event as disclosed herein (e.g., indel, knock-out), for example.

The term “Drosha” as used herein refers to a nuclear RNase III that cleaves primary miRNAs to release hairpin-shaped pre-miRNAs that are subsequently cut by the cytoplasmic RNase III Dicer to generate mature miRNAs.

The terms “drying agent” or “desiccant” as used herein refers to a substance that has an affinity for water such that it will extract the water from other materials.

The term “emollient” as used herein refers to fats or oils in a two-phase system (meaning one liquid is dispersed in the form of small droplets throughout another liquid). Emollients soften the skin by forming an occlusive oil film on the stratum corneum, preventing drying from evaporation in the deeper layers of skin. Thus, emollients are employed as protectives and as agents for softening the skin, rendering it more pliable. Emollients also serve as vehicles for delivery of hydrophobic compounds.

As used herein “emulsion” refers to a colloid system in which both the dispersed phase and the dispersion medium are immiscible liquids where the dispersed liquid is distributed in small globules throughout the body of the dispersion medium liquid. A stable basic emulsion contains at least the two liquids and an emulsifying agent. Common types of emulsions are oil-in-water, where oil is the dispersed liquid and an aqueous solution, such as water, is the dispersion medium, and water-in-oil, where, conversely, an aqueous solution is the dispersed phase. It also is possible to prepare emulsions that are nonaqueous. Creams of the oil-in-water type include hand creams and foundation creams. Water-in-oil creams include cold creams and emollient creams.

Creams may be diluted only with suitable diluents specified in the appropriate entries, and diluted creams must be freshly prepared without the application of heat. Creams should be stored in a cool place and supplied in well-closed containers that prevent evaporation and contamination of the contents. When making a natural cream, however, butters first are melted. The vessel is removed from the heat and the oils are added. When the solution is 100 degrees F, the balance of the liquid portion of the formula then is slowly added while continuously stirred.

The term “encapsulated” as used herein refers to being enclosed in a capsule (meaning a membranous envelope enclosing a part).

The term “endogenous” as used herein refers to that which is naturally occurring, incorporated within, housed within, adherent to, attached to, or resident in.

As used herein, the term “enrich” is meant to refer to increasing the proportion of a desired substance, for example, to increase the relative frequency of a subtype of cell or cell component compared to its natural frequency in a cell population. Positive selection, negative selection, or both are generally considered necessary to any enrichment scheme. Selection methods include, without limitation, magnetic separation and fluorescence-activated cell sorting (FACS).

The term “entrainment” as used herein refers to a process of coordinating the internal circadian clock to external rhythmic time-cues (Zeitgeber), mainly light. The organization of circadian phases is dependent on the geographical and seasonal context and individual clock properties (personal chronotypes). [Schmal, C. et al. Front. Physiol. (2020) 11: 272].

The term “ELISA” or “enzyme-linked immunosorbent assay” as used herein, refers to a rapid, plate-based laboratory technique designed to detect and quantify soluble substances such as peptides, proteins, antibodies, and hormones. In one embodiment of an ELISA, the antigen (target macromolecule) is immobilized on a solid surface (microplate) and then complexed with an antibody that is linked to a reporter enzyme. Detection is accomplished by measuring the activity of the reporter enzyme via incubation with the appropriate substrate to produce a measurable product.

The term “ELISpot”, “enzyme-linked immune absorbent spot”, or “enzyme linked immunospot” as used herein refers to a technique that was developed for the detection of secreted proteins, such as cytokines and growth factors. It is performed using a PVDF or nitrocellulose membrane 96-well plate pre-coated with an antibody specific to the secreted protein. Cells are added to the plate and attach to the coated membrane. Cells are then stimulated, and the secreted protein binds to the antibody. Next, a detection antibody is added that binds specifically to the bound protein. The resulting antibody complex can be detected either through enzymatic action to produce a colored substrate or with fluorescent tags. The membrane can be analyzed by manually counting the spots or with an automated reader designed for this purpose. Each secreting cell appears as a spot of color or fluorescence.

The term “epigenetic marks” as used herein refer to DNA methylation, histone modifications, chromatin remodeling and microRNA. A wide range of post-translational modifications can mark histones, such as acetylation, methylation, ubiquitination or phosphorylation. Histone modifications bring about chromatin remodeling by two main mechanisms. First, marks such as acetylation or phosphorylation neutralize the positive charge of lysines, weakening interactions among nucleosomes and between DNA and histones to increase chromatin accessibility [Bennett, R L and Licht, JD. Annu. Rev. Pharmacol. Toxicol. (2018) 58: 187-207, citing Luger, K. and Richmond, TJ. Curr. Opin. Genetics & Devel. (1998) 8: 140-6; Steger, D J and Workman, J L. BioEssays: news and reviews in molecular, cellular and devel. biol. (1996) 18: 875-84]. Second, histone modifications may serve as docking sites for additional histone modifying enzymes or specific chromatin factors that regulate chromatin architecture and/or gene expression.

Unlike regulation of genetic events, epigenetic regulation is a dynamic and reversible process. Proteins that carry out these epigenetic modifications can be thought of as writers, readers and erasers. Epigenetic writers catalyze the addition of epigenetic marks onto either DNA or histones, most commonly on “tails” of histones that extend from the octamer structure. Readers recognize or are recruited to a specific epigenetic mark. Erasers remove epigenetic marks.

The term “epigenetics” as used herein refers to a stably heritable phenotype resulting from changes in a chromosome without alterations in the DNA sequence. Epigenetic gene expression patterns that cannot be attributed to DNA sequence variations are tightly controlled by the 3-dimensional architecture of chromatin and the action of multi-protein complexes, especially RNA polymerase, which transcribes DNA into RNA. In the nucleus, genomic DNA is wrapped around histones into nucleosome subunits that are condensed into chromatin. Highly condensed chromatin, termed heterochromatin, contains mostly inactive genes. In contrast, euchromatin has a more open structure and contains active genes. Chromatin structure is dynamically regulated by DNA methylation, nucleosome positioning, and histone modifications [Bennett, R L and Licht, JD. Targeting Epigenetics in Cancer. Annu. Rev. Pharmacol. Toicol. (2018) 58: 187-207].

The term “Endosomal Sorting Complexes required for transport” or “ESCRTs” refers to components involved in multivesicular body (MVB) and intraluminal vesicle (ILV) biogenesis. ESCRTs consist of approximately twenty proteins that assemble into four complexes (ESCRT-0, -I, -II and -III) with associated proteins (VPS4, VTA1, ALIX), which are conserved from yeast to mammals (Colombo, M. et al. J. Cell Science (2013) 126: 5553-5565, citing Henne, W. M., et al. (2011). Dev. Cell 21, 77-91; Henne et al., 2011; Roxrud, I. et al. (2010). ESCRT & Co. Biol. Cell 102, 293-318). The ESCRT-0 complex recognizes and sequesters ubiquitylated proteins in the endosomal membrane, whereas the ESCRT-I and -II complexes appear to be responsible for membrane deformation into buds with sequestered cargo, and ESCRT-III components subsequently drive vesicle scission (Id., citing Hurley, J. H. and Hanson, P. I. (2010). Nat. Rev. Mol. Cell Biol. 11, 556-566; Wollert, T. et al. Nature (2009) 458: 172-177). ESCRT-0 comprises hepatocyte growth factor-regulated tyrosine kinase dubstrate (HRS) protein that recognizes the mono-ubiquitylated cargo proteins and associates in a complex with signal-transducing adaptor molecule (STAM), Eps15 and clathrin. HRS recruits TSG101 of the ESCRT-I complex, and ESCRT-I is then involved in the recruitment of ESCRT-III, through ESCRT-II or ALIX, an ESCRT-accessory protein. Finally, the dissociation and recycling of the ESCRT machinery requires interaction with the ATPase associated with various cellular activities (AAA-ATPase) Vps4; Vps4 releases ESCRT-III from the MVB membrane for additional sorting events. It is unclear whether ESCRT-II has a direct role in ILV biogenesis or whether its function is limited to particular cargo (Id., citing Bowers, K. et al. (2006) J. Biol. Chem. 281, 5094-5105; Malerod, L. et al. Traffic 8, 1617-1629).

Concomitant depletion of ESCRT subunits belonging to the four ESCRT complexes does not totally impair the formation of MVBs, indicating that other mechanisms may operate in the formation of ILVs and thereby of exosomes and/or extracellular vesicles (Id., citing Stuffers, S. et al., (2009) Traffic 10, 925-937). One of these pathways requires a type II sphingomyelinase that hydrolyses sphingomyelin to ceramide (Id., citing Trajkovic, K. et al. (2008) Science 319, 1244-1247). Although the depletion of different ESCRT components does not lead to a clear reduction in the formation of MVBs and in the secretion of proteolipid protein (PLP) associated to exosomes and/or extracellular vesicles, silencing of neutral sphingomyelinase expression with siRNA or its activity with the drug GW4869 decreases exosome and/or extracellular vesicle formation and release. However, whether such dependence on ceramides is generalizable to other cell types producing exosomes and/or extracellular vesicles and additional cargos has yet to be determined. The depletion of type II sphingomyelinase in melanoma cells does not impair MVB biogenesis (Id., citing van Niel, G. et al., (2011) Dev. Cell 21, 708-721) or exosome and/or extracellular vesicle secretion, but in these cells the tetraspanin CD63 is required for an ESCRT-independent sorting of the luminal domain of the melanosomal protein PMEL (van Niel et al., (2011) Dev. Cell 21, 708-721). Moreover, tetraspanin-enriched domains have been proposed to function as sorting machineries allowing exosome and/or extracellular vesicle formation (Perez-Hernandez, D. et al. J. Biol. Chem. (2013) 288, 11649-11661).

Despite evidence for ESCRT-independent mechanisms of exosome and/or extracellular vesicle formation, proteomic analyses of purified exosomes and/or extracellular vesicles from various cell types have identified ESCRT components (TSG101, ALIX) and ubiquitylated proteins (Id., citing Buschow, S. et al., (2005) Blood Cells Mol. Dis. 35, 398-403; Thery, C. et al., (2006). Curr. Protoc. Cell Biol. Chapter 3, Unit 3.22). It has also been reported that the ESCRT-0 component HRS could be required for exosome and/or extracellular vesicle formation and/or secretion by dendritic cells (DCs), and thereby impact on their antigen-presenting capacity (Id., citing Tamai, K. et al., (2010) Biochem. Biophys. Res. Commun. 399, 384-390). The transferrin receptor (TfR) in reticulocytes that is generally fated for exosome and/or extracellular vesicle secretion, although not ubiquitylated, interacts with ALIX for MVB sorting (Id., citing Geminard, C. et al., (2004). Traffic 5, 181-193). It was also shown that ALIX is involved in exosome and/or extracellular vesicle biogenesis and exosomal sorting of syndecans (a major family of cell surface heparan sulfate proteoglycans (HSPGs) composed of sulfated glycosaminoglycans (GAGs), heparan sulfate (HS) or both HS and chondroitin sulfate (CS), attached covalently to core proteins—see Park, P W. Methods Cell Biol. (2018) 143: 317-333) through its interaction with syntenin (Colombo, M. et al. J. Cell Science (2013) 126: 5553-5565, citing Baietti, M F et al., (2012). Nat. Cell Biol. 14, 677-685). Silencing of genes for two components of ESCRT-0 (HRS, STAM1) and one of ESCRT-I (TSG101), as well as a late acting component (VPS4B) induced consistent alterations in exosome and/or extracellular vesicle secretion [Colombo, M. et al. J. Cell Sci. (2013) 126: 5553-5565].

The term “exogenous” as used herein refers to that which is non-naturally occurring, or that is originating or produced outside of a specific EV, cell, organism, or species.

The term “expand” and its various grammatical forms as used herein refers to a process by which dispersed living cells propagate in vitro in a culture medium that results in an increase in the number or the amount of viable cells.

As used herein, the term “expression” and its various grammatical forms refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. Expression may also refer to the post-translational modification of a polypeptide or protein.

As used herein, the terms “expression level,” “abundance level,” or simply “abundance” refers to an amount of a gene product, (an RNA species, e.g., mRNA or miRNA, or protein molecule) transcribed or translated by a cell, or an average amount of a gene product transcribed or translated across multiple cells. When referring to mRNA or protein expression, the term generally refers to the amount of any RNA or protein species corresponding to a particular genomic locus, e.g., a particular gene. However, in some embodiments, an expression level can refer to the amount of a particular isoform of an mRNA or protein corresponding to a particular gene that gives rise to multiple mRNA or protein isoforms.

The term “extracellular matrix” as used herein refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fibronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached one or more glycosaminoglycans.

As used herein, the term “extracellular matrix remodeling” refers to the dynamic and tightly regulated process by which the structural network of proteins and polysaccharides surrounding cells is continuously degraded, reorganized, and replaced. The extracellular matrix (ECM) comprises components such as collagen, elastin, proteoglycans, and glycoproteins that collectively provide support and regulate cell signaling. Remodeling involves a balance between enzymatic degradation of existing matrix components, primarily mediated by proteolytic enzymes including metalloproteinases (MMPs), and the synthesis and deposition of new matrix molecules by resident cells. This process is essential for normal physiological functions, including tissue development, wound healing, and adaptation to mechanical stress. Dysregulation of ECM remodeling can contribute to pathological conditions such as fibrosis, tumor progression, and degenerative diseases, wherein excessive matrix deposition or degradation disrupts normal tissue architecture and function (Sonbol, H. S. Journal of microscopy and ultrastructure 6.3 (2018): 123-128, Larsen, M., et al. (2006). Current opinion in cell biology, 18(5), 463-471) Extracellular vesicles (EVs) contribute to ECM remodeling by acting as carriers of bioactive molecules that regulate both matrix degradation and synthesis. EVs released from a variety of cell types can contain proteolytic enzymes, such as MMPs, as well as regulators of these enzymes, thereby directly influencing ECM breakdown. In addition, EVs transport signaling molecules that modulate the behavior of recipient cells. Through these signals, EVs can induce changes in gene expression that promote the production, deposition, or reorganization of ECM components, such as collagen, elastin, and fibronectin. (Nawaz, M., et al. (2018). Cells, 7(10), 167).

The term “extracellular vesicles (EVs)” as used herein refers to nanosized, membrane-bound vesicles released from mammalian cells that can transport cargo-including DNA, RNA, and proteins—between cells as a form of intercellular communication. Different EV types, including microvesicles (MVs), exosomes, oncosomes, and apoptotic bodies, have been characterized on the basis of their biogenesis or release pathways. Microvesicles bud directly from the plasma membrane, are 100 nanometers (nm) to 1 micrometer (m) in size, and contain cytoplasmic cargo (Zaborowski, M P., et al. BioScience (2015) 65 (8): 783-797, citing Heijnen, H F., et al. Blood (1999) 94: 3791-3799). Another EV subtype, exosomes, is formed by the fusion between multivesicular bodies and the plasma membrane, by which multivesicular bodies release smaller vesicles (exosomes) whose diameters range from 40 to 120 nm (Id., citing El Andaloussi, S., et al. Nature Reviews Drug Discovery (2013) 12: 347-357; Cocucci, E. and Meldolesi J. Trends in Cell Biology (2015) 25: 364-372). Dying cells, release vesicular apoptotic bodies (50 nm-2 m) that can be more abundant than exosomes and or extracellular vesicles or MVs under specific conditions and can vary in content between biofluids (Id., citing Thery, C., et al. J. Immunology (2001) 1666: 7309-7318; El Andaloussi, S., et al. Nature Reviews Drug Discovery (2013) 12: 347-357). Membrane protrusions can also give rise to large EVs, termed oncosomes (1-10 m), which are produced primarily by malignant cells in contrast to their nontransformed counterparts (Id., citing Di Vizio, D., et al. Am. J. Pathol. (2012) 181: 1573-1584; Morello, M., et al. Cell Cycle (2013) 12: 3526-3536).

Biogenesis. Exosomes and/or extracellular vesicles are generated in a process that involves double invagination of the plasma membrane and the formation of intracellular multivesicular bodies (MVBs) containing intraluminal vesicles (ILVs). ILVs are ultimately secreted as exosomes and/or extracellular vesicles with a size range of ~40 to 160 nm in diameter through MVB fusion to the plasma membrane and exocytosis. The first invagination of the plasma membrane forms a cup-shaped structure that includes cell-surface proteins and soluble proteins associated with the extracellular milieu. This leads to the de novo formation of an early-sorting endosome (ESE) and in some cases may directly merge with a preexisting ESE. The trans-Golgi network and endoplasmic reticulum can also contribute to the formation and the content of the ESE (Kalluri, R., LeBleu, VS. Science (2020) 367 (6478): eaau6977, citing Kalluri, R. J. Clin. Invest. (2016) 126: 1208-1215; van Neil, G. et al. Nat. Rev. Mol. Cell Biol. (2018) 19: 213-228; McAndrews, KM, Kalluri, R. Mol. Cancer (2019) 18: 52; Mathieu, M. et al. Nat. Cell Biol. (2019) 21: 9-17; Willms, E. et al. Front. Immunol. (2018) 9: 738; Hessvik, NP, Llorente, A. Cell Mol. Life Sci. (2018) 75: 193-208). ESEs can mature into late-sorting endosomes (LSEs) and eventually generate MVBs, which are also called multivesicular endosomes. MVBs form by inward invagination of the endosomal limiting membrane (that is, double invagination of the plasma membrane). This process results in MVBs containing several ILVs (future exosomes and/or extracellular vesicles). The MVB can either fuse with lysosomes or autophagosomes to be degraded or fuse with the plasma membrane to release the contained ILVs as exosomes and/or extracellular vesicles [Id., citing Kahler, C., Kalluri, R. J. Mol. Med. (2013) 91: 431037].

Heterogeneity: The heterogeneity of extracellular vesicles is thought to be reflective of their size, content, functional impact on recipient cells, and cellular origin. During their secretion they acquire surface proteins from their cell of origin. They naturally transport mRNA, miRNA, and proteins between cells.

Biomarkers. There is general agreement that their membranes are specifically enriched in tetraspanins CD9, CD63, CD81, and CD82.

Role. Extracellular vesicles are mediators of near and long-distance intercellular communication in health and disease and affect various aspects of cell biology.

As used herein the term “extraction of nucleic acid” or “nucleic acid extraction”, from a biological sample refers to disruption of the cells in the biological sample, isolation of the nucleic acid from the rest of the cell components, and purification of the nucleic acid, wherein the nucleic acid is RNA or DNA.

The term “eyebrow” refers to an area of coarse skin hairs above the eye that follows the shape of the brow ridges.

The terms “eyelash” and “lash” are used interchangeably to refer to one of the hairs that grow at the edge of the eyelid.

The term “fixed-bed bioreactor”, as used herein, refers to a system configured for culturing cells on a stationary support matrix within a vessel wherein the cells are immobilized or the matrix and a liquid culture medium is passed over or through the matrix to provide nutrients and remove waste. In a fixed-bed bioreactor, the support matrix comprises porous materials fibers, beads, or scaffolds, that enable cell attachment and growth under controlled conditions

The term “flavonoids” as used herein refers to a large family of polyphenols which include several classes based on their basic structure: flavanones, flavones, isoflavones, flavonols, flavanols, and anthocyanins. They have a multitude of biological properties, such as anti-inflammatory, antioxidant, antiviral, antimicrobial, anticancer, cardioprotective, and neuroprotective effects [Liga, S. et al. Plants (2023) 12 (14): 2732].

The term “flow cytometry” as used herein, refers to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Flow analysis and differentiation of the cells is based on size, granularity, and whether the cell is carrying fluorescent molecules in the form of either antibodies or dyes. As the cell passes through the laser beam, light is scattered in all directions, and the light scattered in the forward direction at low angles (0.5-10°) from the axis is proportional to the square of the radius of a sphere and so to the size of the cell or particle. Light also may enter the cell; thus, the 900 light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population (Marion G. Macey, Flow cytometry: principles and applications, Humana Press, 2007). Fluorescence-activated cell sorting (FACS), which allows isolation of distinct cell populations too similar in physical characteristics to be separated by size or density, uses fluorescent tags to detect surface proteins that are differentially expressed, allowing fine distinctions to be made among physically homogeneous populations of cells.

The term “fluidized-bed bioreactor” as used herein, refers to a system configured for culturing cells associated with particulate carriers (e.g., activated carbon, synthetic polymers, porous materials) within a vessel, wherein the carriers are suspended and maintained in a fluidized state by an upward flow of liquid medium and or gas. The fluidization of the carriers promotes mixing and mass transfer while allowing cells attached to the carriers to grow under controlled conditions.

The term “fluorescence” as used herein refers to the result of a three-state process that occurs in certain molecules, generally referred to as “fluorophores” or “fluorescent dyes,” when a molecule or nanostructure relaxes to its ground state after being electrically excited. Stage 1 involves the excitation of a fluorophore through the absorption of light energy; Stage 2 involves a transient excited lifetime with some loss of energy; and Stage 3 involves the return of the fluorophore to its ground state accompanied by the emission of light.

The terms “formulation” and “composition” are used interchangeably herein to refer to a product of the described invention that comprises all active and inert ingredients.

As used herein the term “fragment” and its other grammatical forms are meant to refer to portions of a nucleic acid, polynucleotide or oligonucleotide shorter than the full sequence of a reference molecule. The sequence of bases in a fragment is unaltered from the sequence of the corresponding portion in the molecule from which it arose; there are no insertions or deletions in a fragment in comparison with the corresponding portion of the molecule from which it arose. As contemplated herein, a fragment of a nucleic acid or polynucleotide, such as an oligonucleotide, is 15 or more bases in length, or 16 or more, 17 or more, 18 or more, or 19 or more, or 20 or more, or 21 or more, or 22 or more, or 23 or more, or 24 or more, or 25 or more, or 26 or more, or 27 or more, or 28 or more, or 29 or more, 30 or more, 50 or more, 75 or more, 100 or more bases in length, up to a length that is one base shorter than the full length sequence.

The term “free radical” as used herein refers to a highly reactive and usually short-lived molecular fragment with one or more unpaired electrons. Free radicals are highly chemically reactive molecules. Because a free radical needs to extract a second electron from a neighboring molecule to pair its single electron, it often reacts with other molecules, which initiates the formation of many more free radical species in a self-propagating chain reaction. This ability to be self-propagating makes free radicals highly toxic to living organisms. Oxidative injury may lead to widespread biochemical damage within the cell. The molecular mechanisms responsible for this damage are complex. For example, free radicals may damage intracellular macromolecules, such as nucleic acids (e.g., DNA and RNA), proteins, and lipids. Free radical damage to cellular proteins may lead to loss of enzymatic function and cell death. Free radical damage to DNA may cause problems in replication or transcription, leading to cell death or uncontrolled cell growth. Free radical damage to cell membrane lipids may cause the damaged membranes to lose their ability to transport oxygen, nutrients or water to cells.

The term “gene” as used herein refers to a DNA polynucleotide sequence that expresses an RNA (RNA is transcribed from the DNA polynucleotide sequence) from a coding region, which RNA can be a messenger RNA (encoding a protein) or a non-protein-coding RNA. A “gene” can refer to the coding region alone or may include regulatory sequences upstream and/or downstream to the coding region (e.g., promoters, 5′-untranslated regions, 3′-transcription terminator regions). A coding region encoding a protein can alternatively be referred to herein as an “open reading frame” (ORF). A gene that is “native” or “endogenous” refers to a gene as found in nature with its own regulatory sequences; such a gene is located in its natural location in the genome of a host cell. A “chimeric” gene refers to any gene that is not a native gene, comprising regulatory and coding sequences that are not found together in nature (i.e., the regulatory and coding regions are heterologous with each other). Accordingly, a chimeric gene may comprise regulatory sequences and coding sequences that are derived from different sources, or regulatory sequences and coding sequences derived from the same source but arranged in a manner different than that found in nature. A “foreign” or “heterologous” gene refers to a gene that is introduced into the host organism by gene transfer. Foreign/heterologous genes can comprise native genes inserted into a non-native organism, native genes introduced into a new location within the native host, or chimeric genes. A homologous gene is a type of gene that is inherited by two different species that evolved from the same ancestor. A paralogue gene is one of a set of homologous genes that have diverged from each other as a consequence of genetic duplication. A “transgene” is a gene that has been introduced into the genome by a gene delivery procedure (e.g., transformation). A “codon-optimized” open reading frame has its frequency of codon usage designed to mimic the frequency of preferred codon usage of the host cell.

The term “growth factor” as used herein refers to extracellular polypeptide molecules that bind to a cell-surface receptor triggering an intracellular signaling pathway, leading to proliferation, differentiation, or other cellular response. These pathways stimulate the accumulation of proteins and other macromolecules, e.g., by increasing their rate of synthesis, decreasing their rate of degradation, or both.

The term “hair health” as used herein refers to shiny hair with a smooth texture and clean-cut ends or tapered tips. Hair consists of an outer hydrophobic lipid epicuticle, a layer of flattened overlapping cuticle cells surrounding the elongated polyhedral cortical cells. The normal cuticle, which has a smooth appearance, allowing light reflection and limiting friction between the hair shafts, is responsible for the luster and texture of the hair [Sinclair, RD. J. Investig. Dermatology Symposium Proceedings (2007) 12: 2-5, citing Draelos, Z D. Dermatol. Clin. (1991) 9: 199-227]. The cortical layer determines many of the mechanical properties of the hair. It consists of closely packed spindle-shaped cortical cells filled with keratin filaments that are orientated parallel to the longitudinal axis of the hair shaft, and an amorphous matrix of high sulfur proteins [Id., citing Dawber, R. Clin. Dermatol. (1996) 4: 105-112]. The outer, intensely hydrophobic layer and the cortex confer the physical properties of luster (shine) and volume (body) that contribute to the appearance of hair health.

The term “heat stress” or “heat shock” as used herein refers to cellular damage caused by elevated temperatures, often leading to destabilized protein structures, damage to cellular membranes, and disruption of energy metabolism, leading to an accumulation of damaged proteins. Increased exposure to heat renders noncovalent interactions that stabilize biomolecules less favorable, reducing protein stability and promoting protein unfolding or the formation of unstable structures. As molecular motion within cells increases following heat exposure, cell membranes become more fluid, which reduces membrane barrier function and the organization of signaling complexes. Intracellular signaling processes are also affected, as proteins involved in these reactions can misfold or dissociate from binding partners, potentially interfering with pathways governing growth, stress, and survival.

The term “heat shock proteins” or “HSPs” as used herein refers to highly conserved multimolecular complexes expressed constitutively under normal growth conditions in cells that act as molecular chaperones, which play a regulatory role in the folding of proteins, intracellular transport of proteins in cytosol, endoplasmic reticulum and mitochondria, repair or degradation of proteins and refolding of misfolded proteins. In addition to being constitutively expressed, these proteins can be induced by a range of environmental, pathological, or physiological stimuli.

The term “hollow fiber bioreactor” as used herein refers to a high-density, 3D culture system that mimics capillary networks to grow cells. It uses semi-permeable hollow fiber capillary tubes made of polymers to create two spaces: (1) the extracapillary (“cell”) side where the cells are grown; and (2) the intracapillary (“media”) side where media flows at a high rate. The semi-permeable walls of the hollow fibers allow essential nutrients to feed the growing cells from the media side to the cell side. The separation of the cell product from the bulk media flow prevents the cells from experiencing shear stress that could limit growth and production and keeps the product highly concentrated to cut downstream processing requirements. Waste flows from the cell side to the media side to be washed away, but the hollow fiber pore size restrictions retain cells and cell products. Desired cell products (e.g., proteins, extracellular vesicles, etc.) are continually harvested into a refrigerated harvest container as a concentrated, cell-free supernatant that is ready for downstream purification.

The term “homolog” as used herein refers to being similar in form or structure, but not necessarily in function; homology suggests evolutionary relatedness.

The term “hormone” as used herein refers to natural substances produced by organs of the body that travel by blood to trigger activity in other locations or their synthetic analogs. Suitable hormones for use in the context of the described invention include, but are not limited to, calciferol (Vitamin D3) and its products.

The term “hybridization” refers to the process of combining complementary, single-stranded nucleic acids into a single molecule. Nucleotides will bind to their complement under normal conditions, so two perfectly complementary strands will bind (or ‘anneal’) to each other readily. However, due to the different molecular geometries of the nucleotides, a single inconsistency between the two strands will make binding between them more energetically unfavorable. Measuring the effects of base incompatibility by quantifying the rate at which two strands anneal can provide information as to the similarity in base sequence between the two strands being annealed. The term “specifically hybridizes” as used herein refers to the process whereby a nucleic acid distinctively or definitively forms base pairs with complementary regions of at least one strand of DNA that was not originally paired to the nucleic acid. A nucleic acid that selectively hybridizes undergoes hybridization, under stringent hybridization conditions, of the nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, at least 90% sequence identity, or at least 100% sequence identity (i.e., complementary) with each other.

The term “hypopigmenting agents” as used herein refers to substances capable of depigmenting the skin.

The terms “immune response” and “immune-mediated” are used interchangeably herein to refer to any functional expression of a subject's immune system, against either foreign or self-antigens, whether the consequences of these reactions are beneficial or harmful to the subject.

The term “immune system” as used herein refers to a complex arrangement of cells and molecules that maintain immune homeostasis to preserve the integrity of the organism by elimination of all elements judged to be dangerous. Responses in the immune system may generally be divided into two arms, referred to as “innate immunity” and “adaptive immunity.” The two arms of immunity do not operate independently of each other but rather work together to elicit effective immune responses.

The term “immunofluorescence” as used herein, refers to an imaging technique that uses fluorescently labeled antibodies to detect, localize, and quantify specific antigens within cells or tissues.

The term “immunogen” and its various grammatical forms as used herein refers to a substance that elicits an immune response.

The term “immunohistochemistry” as used herein refers to a laboratory technique that uses antibodies to detect specific antigens in tissue sections.

The term “immunological synapse” (“IS”) as used herein refers to a highly structured body that functions to concentrate T cell signaling in a defined area. It is associated with the selective recruitment of signaling molecules and exclusion of negative regulators. The synapse is stabilized by a ring of adhesion molecules, including, for example, LFA1, which binds to ICAM1 on the APC. The immune synapse modulates T cell receptor (TCR) signaling by several mechanisms. In the earliest stages of immune synapse formation, TCR-containing microclusters are recruited to the central molecular cluster. The TCR responds to two distinct pMHC ligands (agonist and self peptide-MHC) in coagonism rather than nonspecific TCR-MHC interactions, which adds to the overall binding strength of the TCR-p-MHC complex. The strength of the TCR-p-MHC interactions has a role in determining the influence of coreceptors that are recruited to the immune synapse (e.g., CD8). The immune synapse also modulates TCR signaling by regulating interactions between kinases in the TCR pathway and their substrates [Morris, G. and Allen, P M. Nature Immunol. (2012) doi:10.1038/nm.2190].

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least about 5%, at least about 10%, at least about 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

The term “inhibitor” as used herein refers to a molecule that reduces the amount or rate of a process, stops the process entirely, or that decreases, limits, or blocks the action or function thereof. Enzyme inhibitors are molecules that bind to enzymes thereby decreasing enzyme activity. Inhibitors may be evaluated by their specificity and potency.

The term “inflammasome” as used herein refers to a pro-inflammatory protein complex that is formed after stimulation of the intracellular nucleotide-binding oligomerization domain (NOD)-like receptors. Production of an active caspase in the complex processes cytokine proteins into active cytokines.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “inflammatory cytokines” or “inflammatory mediators” as used herein refers to the molecular mediators of the inflammatory process, which may modulate being either pro- or anti-inflammatory in their effect. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, pro-inflammatory cytokines, including, but not limited to, interleukin-1-beta (IL-1), interleukin-4 (IL-4), interleukin-6 (IL-6), interleukin-8 (IL-8), tumor necrosis factor-alpha (TNF-α), interferon-gamma (IF-7), and interleukin-12 (IL-12).

The term “innate immunity” as used herein refers to a nonspecific fast response to pathogens that is predominantly responsible for an initial inflammatory response before adaptive immunity is induced. These include such mechanisms as anatomical barriers, antimicrobial peptides, the complement system and the chemokine/cytokine system; macrophages and neutrophils carrying nonspecific pathogen-recognition receptors, and a number of specialized cell types, including innate lymphoid cells (ILCs, including natural killer (NK) cells) mast cells and dendritic cells (DCs). Innate immunity is present in all individuals at all times, does not increase with repeated exposure to a given pathogen, and discriminates between groups of similar pathogens, rather than responding to a specific pathogen.

The term “insulin-like growth factor 1” or IGF-1” as used herein refers to a hormone similar in molecular structure to insulin that has growth-promoting effects on almost every cell in the body, especially skeletal muscle, cartilage, bone, liver, kidney, nerves, skin, hematopoietic cell, and lungs. It plays an important role in childhood growth and continues to have anabolic effects in adults. IGF-1 is produced primarily by the liver as an endocrine hormone as well as in target tissues in a paracrine/autocrine fashion. Production is stimulated by growth hormone (GH) and can be retarded by undernutrition, growth hormone insensitivity, lack of growth hormone receptors, or failures of the downstream signaling molecules, including tyrosine-protein phosphatase non-receptor type 11 (also known as SHP2, which is encoded by the PTPN11 gene in humans) and signal transducer and activator of transcription 5B (STAT5B), a member of the STAT family of transcription factors. Its primary action is mediated by binding to its specific receptor, the Insulin-like growth factor 1 receptor (IGF1R), present on many cell types in many tissues. Binding to the IGF1R, a receptor tyrosine kinase, initiates intracellular signaling. IGF-1 is one of the most potent natural activators of the AKT signaling pathway, a stimulator of cell growth and proliferation, and a potent inhibitor of programmed cell death. IGF-1 is a primary mediator of the effects of growth hormone (GH). Growth hormone is made in the pituitary gland, released into the blood stream, and then stimulates the liver to produce IGF-1. IGF-1 then stimulates systemic body growth. In addition to its insulin-like effects, IGF-1 can regulate cell growth and development, especially in nerve cells, as well as cellular DNA synthesis. IGF-1 was shown to increase the expression levels of the chemokine receptor CXCR4 (receptor for stromal cell-derived factor-1, SDF-1) and to markedly increase the migratory response of mesenchymal stem cells (MSCs) to SDF-1 (Li, Y, et al. 2007 Biochem. Biophys. Res. Communic. 356(3): 780-784). The IGF-1-induced increase in MSC migration in response to SDF-1 was attenuated by PI3 kinase inhibitor (LY294002 and wortmannin) but not by mitogen-activated protein/ERK kinase inhibitor PD98059.

The term “integrin” as used herein refers to heterodimeric cell surface proteins involved in cell-cell and cell-matrix interactions. They are important in adhesive interactions between lymphocytes and antigen-presenting cells and in lymphocyte and leukocyte adherence to blood vessel walls and migration into tissues.

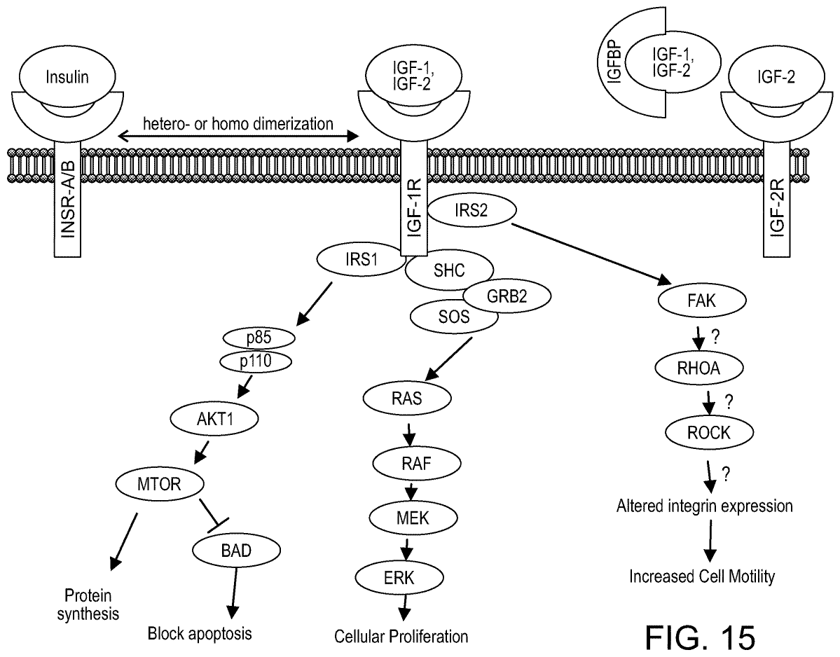

The term “Insulin-Like Growth Factor (IGF) signaling pathway” refers to a complex and tightly regulated network which is critical for cell proliferation and survival [Iams, W T and Lovly, C M. Clin. Cancer Res. (2015) 21 (19): 4270-77, citing (1). This pathway (FIG. 15) is composed of three receptor tyrosine kinases—insulin-like growth factor-1 receptor (IGF-1R), insulin-like growth factor-2 receptor (IGF-2R), and insulin receptor (INSR); three ligands-insulin, IGF-1, and IGF-2 (Id, citing 2, 3); and six serum Insulin-like Growth Factor Binding Proteins (IGFBP's), which serve as regulators of the pathway by determining ligand bioavailability (Id., citing 4). The most prevalent of the IGFBP's is IGFBP3 (5). Both IGF-1 and IGF-2 exert their effects through autocrine, paracrine, and endocrine mechanisms, and both can activate IGF-1R signaling. For simplification, IGF-1 ligand only is shown binding to IGF-1R. IGF-1 binding to IGF-1R promotes receptor homodimerization or heterodimerization with INSR. Ligand-activated IGF-1R first binds to intracellular adaptor proteins, such as insulin receptor substrate1 (IRS1) and SHC. These adaptor proteins transmit signals through the phosphatidyl-inositol-3 kinase (PI3K)-AKT1-mammalian target of rapamycin (MTOR) pathway and through the mitogen activated protein kinase (MAPK) pathway. Activated IGF-1R promotes cellular motility through activation of IRS2, which alters integrin expression through poorly understood mechanisms involving the small G protein RHOA, focal adhesion kinase (FAK), Rho-kinase (ROCK), PI3K, and other signaling molecules. Targets for potential monotherapy and combinatorial therapeutic strategies are noted in the figure. TKI: tyrosine kinase inhibitor. mAb: monoclonal antibody.

The term “interferons” (“IFNs”) as used herein refers to several related families of cytokines originally named for their interference of viral replication. IFN-α and IFN-β are antiviral in their effects; IFN-γ has other roles in the immune system.

The term “IFN-α, IFN-β” and “type I interferons” as used herein refers to antiviral cytokines produced by a wide variety of cells in response to infection by a virus and which also help healthy cells resist viral infection. They act through the same receptor, which signals through a Janus-family tyrosine kinase. Type I interferons are inducible and are synthesized by many cell types after infection by diverse viruses. Almost all types of cells can produce IFN-α and IFN-β in response to activation of several innate sensors. The major producers of IFN-α and -β are macrophages and DCs [Janeway's Immunology, 9th Ed. (2017) Garland Science, New York, at 122-125].

The term “IFNγ” as used herein refers to a cytokine of the interferon structural family produced by effector CD4TH1 cells, CD8 T cells, and NK cells. Its primary function is the activation of macrophages. It acts through a different receptor from that of the type I interferons. IFN-γ is produced by activated Th1 cells and ILC1s, including NK cells, which are activated mainly through TLRs.

The term “IFNλ” or “type III interferons” as used herein refers to a family that includes IL-28A, IL-28B, and IL-29, which bind a common receptor expressed by a limited set of epithelial tissues.

The term “interferon regulatory factor (IRF) as used herein refers to a family of nine transcription factors that regulate a variety of immune responses. For example, IRF3 and IRF7 are activated as a result of signaling from some TLRs. Several IRFs promote expression of the genes for type 1 interferons.

The term “interferon stimulating genes” or “ISGs” as used herein refers to a category of gene induced by interferons, which include many that promote innate defense against pathogens, such as oligoadenylate synthetase, PDR and the Mx, IFITs and IFI™ proteins.

The term “interferon α receptor” or “IFNAR” as used herein refers to a receptor that recognizes IFN-α and IFN-β to activate STAT1 and STAT2 and induce expression of many ISGs.

The term “Interleukin (IL)” as used herein refers to a cytokine secreted by, and acting on, leukocytes. Interleukins regulate cell growth, differentiation, and motility, and stimulates immune responses, such as inflammation. Examples of interleukins include interleukin-1 (IL-1), interleukin 2 (IL-2), interleukin-1β (IL-1β), interleukin-6 (IL-6), interleukin-8 (IL-8), and interleukin-12 (IL-12).

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “Janus kinase (JAK) family” as used herein refers to enzymes of the JAK-STAT intracellular signaling pathways that link many cytokine receptors with gene transcription in the nucleus. The signaling cascade is induced by cytokines that signal via the Jak-STAT signaling pathway. Cytokine binding to a specific receptor allows transactivation of the associated Janus Kinases (JAKs). Activated JAKs then phosphorylate tyrosines on the intracellular domains of the receptor, which recruit the Signal Transducers and Activators of Transcription (STAT) transcription factors. STATs are translocated into the nucleus and upregulate the transcription of cytokine-responsive genes. Suppressors of cytokine signaling (SOCS) proteins are direct targets of STAT and act as negative-feedback inhibitors to switch off the signaling cascade [Morris, R. et al. Protein Science (2018) 27: 1984-2009].

The term “keratolytics” (desquamating agents) as used herein refers to an agent that acts to remove outer layers of the stratum corneum. Keratolytics are particularly useful in hyperkeratotic areas. The keratolytics include, but are not limited to, benzoyl peroxide, fluorouracil, resorcinol, salicylic acid, tretinoin, and the like.

As used herein, the term “Ki-67” refers to a nuclear protein that is expressed exclusively during the active cell cycle and is absent in resting cells and is quantified using anti-Ki-67 antibodies.

As used herein, the term “KITLG” refers to the Kit ligand (KITLG), also known as stem cell factor (SCF), binds to the c-Kit receptor. KITLG regulates hair pigmentation and the differentiation, survival, and migration of mast cells. KITLG is expressed in keratinocytes, fibroblasts and immune cells of the skin. It is induced by UV; it acts as a paracrine factor that regulates the expression of MITF to promote melanin synthesis. KITLG plays an essential role in inducing follicular melanogenesis (hair pigmentation). SCF downregulation causes a dramatic reduction in melanoblast numbers as well as severe defects in hair pigmentation.

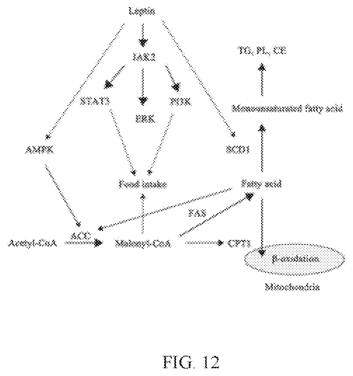

The term “leptin” or “LEP” as used herein refers to a protein hormone secreted by adipose tissue. The function of leptin is multifaceted, mainly in the regulation of fat and body weight; appetite suppression; increased energy consumption; direct inhibition of fat synthesis and promote its decomposition. Insulin can promote the secretion of leptin; leptin decreases insulin synthesis and secretion by pancreatic beta cells and increases insulin hepatic extraction. As a result, insulin delivery is reduced by leptin. The secretion of leptin has a circadian rhythm, which is characterized by high evening, low morning, and impulsive secretion. Leptin exerts its actions by binding to leptin receptors in various tissues, especially in the CNS. Activation of leptin receptor (LepR) with leptin activates signaling modules such as the Janus kinase/signal transducer and activator of transcription (JAK/STAT) pathway, phosphoinositide 3-kinase (PI3K pathway), the mitogen-activated protein kinase (MAPK) pathway; the extracellular signaling-regulated kinase 1/2 (ERK1/2) pathway; the adenosine monophosphate kinase (AMPK) pathway; and the PPAR gamma coactivator/peroxisome proliferator-activated receptor (PGC/PPAR) pathway. Tyrosine phosphorylation of long leptin receptor isoform LEPRb induces binding of STATs to LEPRb. Binding of STATs to the phosphorylated residues of LEPR leads to the JAK2 mediated tyrosine phosphorylation and activation of STATs. Activated STATs translocate to the nucleus and induces expression of genes such as suppressor of cytokine signaling 3 (SOCS3) and TIMP metallopeptidase inhibitor 1 (TIMP1). SOCS3 mediates feedback inhibition of leptin pathway by binding to Tyr-986 residue of LEPR (see, FIG. 12) [Liu, Z., et al. Front. Neurosci. (2023) 17: 1238528].

The term “library” as used herein refers to a collection of recombinant DNA molecules which, together, commonly represents the entire genome of an organism (a “genomic library”). Another type of library consists of a collection of cDNA molecules derived from a mixture of polyadenylated mRNAs isolated from a population of cells of a given strain; this kind of library does not represent the entire genome of the strain.

As used herein, the term “liquid biopsy” sample refers to any sample taken from a subject, which can reflect a biological state associated with the subject. Examples of sources of liquid biopsy samples include, but are not limited to, blood, whole blood, plasma, serum, urine, cerebrospinal fluid, fecal, saliva, sweat, tears, pleural fluid, pericardial fluid, or peritoneal fluid of the subject. A liquid biopsy sample can include any tissue or material derived from a living or dead subject. A liquid biopsy sample can be a cell-free sample. A liquid biopsy sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof.

As used herein, the term “locus” refers to a position (e.g., a site) within a genome, e.g., on a particular chromosome. In some embodiments, a locus refers to a single nucleotide position, on a particular chromosome, within a genome. In some embodiments, a locus refers to a group of nucleotide positions within a genome. In some instances, a locus is defined by a mutation (e.g., substitution, insertion, deletion, inversion, or translocation) of consecutive nucleotides within a cancer genome. In some instances, a locus is defined by a gene, a sub-genic structure (e.g., a regulatory element, exon, intron, or combination thereof), or a predefined span of a chromosome. Because normal mammalian cells have diploid genomes, a normal mammalian genome (e.g., a human genome) will generally have two copies of every locus in the genome, or at least two copies of every locus located on the autosomal chromosomes, e.g., one copy on the maternal autosomal chromosome and one copy on the paternal autosomal chromosome. As used herein, the term “allele” refers to a particular sequence of one or more nucleotides at a chromosomal locus. In a haploid organism, the subject has one allele at every chromosomal locus. In a diploid organism, the subject has two alleles at every chromosomal locus.

The term “long noncoding RNA” (“lncRNAs”) as used herein refers to a class of transcribed RNA molecules that are longer than 200 nucleotides and yet do not encode proteins. LncRNAs can fold into complex structures and interact with proteins, DNA and other RNAs, modulating the activity, DNA targets or partners of multiprotein complexes. Crosstalk of lncRNAs with miRNAs creates an intricate network that exerts post-transcriptional regulation of gene expression. For example, lncRNAs can harbor miRNA binding sites and act as molecular decoys or sponges that sequester miRNAs away from other transcripts. Competition between lncRNAs and miRNAs for binding to target mRNAs has been reported and leads to de-repression of gene expression (Zampetaki, A., et al. Front. Physiol. (2018) doi.org/10.3389/fphys.2018.01201, citing Yoon, J H., et al. Semin. Cell Dev. Bio. (2014) 34: 9-14; Ballantyne, MD., et al. Clin. Pharmacol. Ther. (2016) 99: 494-501). Finally, lncRNAs may contain embedded miRNA sequences and serve as a source of miRNAs (Id., citing Piccoli, MT., et al. Cir. Res. (2017) 121: 575-583).

The term “low density lipoprotein receptor related protein 1” or “LRP1” as used herein refers to a transmembrane receptor protein belonging to the LDL receptor family, which plays multifunctional roles in maintaining endocytosis, homeostasis and signal transduction. Accumulating evidence suggests that LRP1 modulates vascular homeostasis mainly by regulating vasoactive substances and specific intracellular signaling pathways, including the plasminogen activator inhibitor 1 (PAI-1) signaling pathway, platelet-derived growth factor (PDGF) signaling pathway, transforming growth factor-β (TGF-β) signaling pathway and vascular endothelial growth factor (VEGF) signaling pathway [He, Z. et al. Biomedicine & Pharmacotherapy (2021) 139: 111667].

The term “macrophage” as used herein refers to a mononuclear, actively phagocytic cell arising from monocyte stem cells in the bone marrow. These cells are widely distributed in the body and vary in morphology and motility. Phagocytic activity is typically mediated by serum recognition factors, including certain immunoglobulins and components of the complement system, but also may be nonspecific. Macrophages also are involved in both the production of antibodies and in cell-mediated immune responses, particularly in presenting antigens to lymphocytes. They secrete a variety of immunoregulatory molecules. Macrophages have been classified based on their mode of activation: classically activated/M1 macrophages respond to interferon-gamma (IFN-γ) by releasing pro-inflammatory cytokines and are involved in TH1 cell mediated resolution of acute infection. Alternatively activated/M2 macrophages respond to cytokines from TH2 cells and are involved in wounding and fibrosis [Ghajar, C M., et al., “The role of the microenvironment in tumor initiation, progression, and metastasis,” In Mendelsohn, J., et al., the Molecular Basis of Cancer, Elsevier Saunders, Philadelphia, citing Pollard, J W, Nat. Rev. Immunol. (2009) 9: 259-270]. The diverse functions of macrophages are executed in a tissue- and context-specific fashion by a number of discrete macrophage subtypes, which aid these developmental processes by remodeling collagen and secreting a host of pro-angiogenic, pro-inflammatory and matrix-degrading factors (Id., citing Qian, B Z, Pollard, J W. Cell (2010) 141: 39-51).

The term “MagPix™ multiplex assay” as used herein refers to a bead-based and imaging based immunoassay platform that is used to simultaneously measure up to 50 different proteins, DNA, or RNA analytes in a single sample (see, Li Z N, et al. 2017 May; 11(3):289-297. doi: 10.1111/irv.12449. Epub 2017 Apr. 5).

The terms “Major Histocompatibility Complex (MHC), MHC-like molecule” and “HLA” are used interchangeably herein to refer to cell-surface molecules that display a molecular fraction known as an epitope or an antigen and mediate interactions of leukocytes with other leukocyte or body cells. MHCs are encoded by a large gene group and can be organized into three subgroups-class I, class II, and class III. In humans, the MHC gene complex is called HLA (“Human leukocyte antigen”); in mice, it is called H-2 (for “histocompatibility”). Both species have three main MHC class I genes, which are called HLA-A, HLA-B, and HLA-C in humans, and H2-K, H2-D and H2-L in the mouse. These encode the α chain of the respective MHC class I proteins. The other subunit of an MHC class I molecule is β2-microglobulin. The class II region includes the genes for the α and β chains (designated A and B) of the MHC class II molecules HLA-DR, HLA-DP, and HLA-DQ in humans. Also in the MHC class II region are the genes for the TAPI:TAP2 peptide transporter, the PSMB (or LMP) genes that encode proteasome subunits, the genes encoding the DMa and BMP chains (DMA and DMB), the genes encoding the α and β chains of the DO molecule (DOA and DOB, respectively), and the gene encoding tapasin (TAPBP). The class II genes encode various other proteins with functions in immunity. The DMA and DMB genes encoding the subunits of the HLA-DM molecule that catalyze peptide binding to MHC class II molecules are related to the MHC class II genes, as are the DOA and DOB genes that encode the subunits of the regulatory HLA-DO molecule [Janeways Immunobiology. 9th ed., GS, Garland Science, Taylor & Francis Group, 2017. pps. 232-233]. MHC-like molecules, while not encoded by the same gene group as true MHCs, have the same folding and overall structure of MHCs, and specifically MHC class I molecules, and thus possesses similar biological functions such as antigen presentation. MHC Class I-like molecules are nonclassical MHC type molecules, while including Cd1d also include CD1a, CD1b, CD1c, CD1e, and MR1 are also expressed on APCs and can activate various subsets of T cells [Kumar and Delovitch (2014) “Different subsets of natural killer T cells may vary in their roles in health and disease.” Immunology 142: 321-336]. Other non-classical histocompatibility molecules include MR1, which activate MAIT cells.

The term “MHC restriction” as used herein refers to the requirement that APCs or target cells express MHC molecules that a T cell recognizes as self in order for T cell to respond to the antigen presented by that APC or target cell (T cells will only recognize antigens presented by their own MHC molecules). For example, CD8 T cells bind class I MHC which are expressed on most cells in the body, and CD4 T cells bind class II MHC which are only expressed on specialized APCs.

The term “mammalian exosomes” as used herein refers to extracellular bilayered membrane-bound vesicles of endosomal origin in a size range of ~40 to 160 nm in diameter (~100 nm on average) generated by all mammalian cells that are actively secreted.

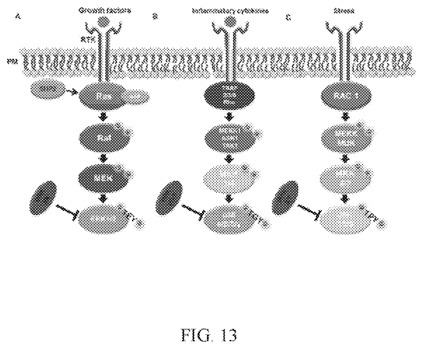

The abbreviation “MAPK” as used herein refers to Mitogen-Activated Protein Kinase (MAPK) signaling, which activates a three-tiered cascade with MAPK kinase kinases (MAP3K) activating MAPAK kinases (MAP2K) and finally MAPK. MAPKs are protein Ser/Thr kinases that convert extracellular stimuli into a wide range of cellular responses [Cargnello, M. and Roux, PP, Microbiol. Mol. Biol. Rev. (2011) 75(1): 50-83]. FIG. 13 is a schematic of the MAPK signaling pathways [taken from Soares-Silva, M., et al. Front. Microbiol. (2016) 7: 183]. The major MAPK pathways involved in inflammatory diseases are extracellular regulating kinase (ERK), p38 MAPK, and c-Jun NH2-terminal kinase (JNK). All three MAPK pathways may be activated by TGF-β, and signaling through these cascades can further regulate the expression of Smad proteins and mediate Smad-independent TGF-β responses. These three MAPK pathways are all involved in TGF-β-induced fibrosis [He, W. and Dai, C. Curr. Pathobiol. Rep. (2015) 3: 183-92, citing Tsou, P S., et al. Am. J. Physiol. Cell Physiol. (22014) 307: C2-C13; Kamato, D., et al. Cell Signal (2013) 25: 2017-2024; Pannu, J., et al. J. Biol. Chem. (2007) 282: 10405-10413; Yu, L., et al. J. Biol. Chem. (2002) EMBO J. 21: 3749-3759]. Upstream kinases include TGFβ-activated kinase-1 (TAKI) and apoptosis signal-regulating kinase-1 (ASKI). Downstream of p38 MAPK is MAPK activated protein kinase 2 (MAPKAPK2 or MK2). TGF-β can signal in a noncanonical manner via the MAPK family.

The term “matrix metalloproteinases” as used herein refers to a collection of zinc-dependent proteases involved in the breakdown and the remodeling of extracellular matrix components (Guiot, J., et al. Lung (2017) 195(3): 273-280, citing Oikonomidi, et al. Curr Med Chem. 2009; 16(10): 1214-1228). MMP-1 and MMP-7 seem to be primarily overexpressed in plasma of IPF patients compared to hypersensitivity pneumonitis, sarcoidosis and COPD with a possible usefulness in differential diagnosis (Id., citing Rosas I O., et al. PLoS Med. 2008; 5(4): e93). They are also involved in inflammation and seem to take part to the pathophysiological process of pulmonary fibrosis (Id., citing Vij R, Noth I. Transl Res. 2012; 159(4): 218-227; Dancer R C A., et al. Eur Respir J. 2011; 38(6): 1461-1467). The most studied is MMP-7, which is known as being significantly increased in epithelial cells both at the gene and protein levels and is considered to be active in hyperplastic epithelial cells and alveolar macrophages in IPF (Id., citing Fujishima S., et al. Arch Pathol Lab Med. (2010) 134(8): 1136-1142). There is also a significant correlation between higher MMP-7 concentrations and disease severity assessed by forced vital capacity (FVC) and diffusing capacity of the lungs for carbon monoxide (DLCO) (Id., citing Rosas I O., et al. PLoS Med. 2008; 5(4): e93). Higher levels associated to disease progression and worse survival (>4.3 ng/ml for MMP-7) (Id.). The MMP2 gene provides instructions for making matrix metallopeptidase 2. This enzyme is produced in cells throughout the body and becomes part of the extracellular matrix, which is an intricate lattice of proteins and other molecules that forms in the spaces between cells. One of the major known functions of MMP-2 is to cleave type IV collagen, which is a major structural component of basement membranes, the thin, sheet-like structures that separate and support cells as part of the extracellular matrix.

MMPs play a critical role in neuroinflammation through the cleavage of ECM proteins, cytokines and chemokines. (Ji. R-R., et al, US Neurology, Touch Briefings (2008) 71-74). MMP-2 is constitutively expressed and normally present in brain and spinal cord tissues. In contrast, MMP-9 is normally expressed at low levels, but upregulated in many injury and disease states such as spinal cord injury and brain trauma (Id., citing Rosenberg, GA. Glia (2002) 39: 279-291); it is also induced in the crushed sciatic nerve and causes demyelination, a condition associated with neuropathic pain, by the cleavage of myelin basic protein. (Id., citing Chattopadhyay, S., et al. Brain Behav. Immun. (20007) 21: 561-568). Besides targeting matrix, because MMPs can process a variety of growth factors and other extracellular cytokines and signals, they may contribute to the neurovascular remodeling that accompanies chronic CNS injury. (Id., citing Zhao, B Q., et al. Nat. Med. (2006) 12: 441-445).

The term “messenger RNA” (“mRNA”) as used herein refers to a coding RNA that functions in protein translation.

The term “microfluidic device” as used herein refers to small systems that manipulate minute amounts of fluid through microscopic channels, typically designed on chips. Microfluidic devices enable precise and sensitive measurements of EV analysis with small sample volumes, using an array of quantification techniques (e.g., fluorescence, biomarker detection, electrochemistry, magnetic sensing, etc.). Microfluidic systems provide faster, more accessible alternatives for EV quantification (Calero V, et al. Extracell Vesicles Circ Nucl Acids. 2025 Sep. 12; 6(3):560-579).

The term “microRNA,” “miRNA”, or “miR” as used herein refers to a class of small, non-coding RNA molecules, usually from about 18 to about 28 nucleotides in length. MicroRNAs are partially complementary to one or more messenger RNA (mRNA) molecules, and function in posttranscriptional regulation of gene expression and RNA silencing. A precursor microRNA (miRNA) has two arms: miR-5p and miR-3p (miR-5p/-3p). In the precursor miRNA stem loop structure, the 5p strand is present in the forward (5′-3′) position and the 3p strand (which will be almost complimentary to the 5p strand) is located in the reverse position. In terms of which strand is functional following Dicer cleavage of the stem loop to produce the two mature strands, either the 5p or 3p strand or both can become functional depending on the tissue or cell types. The stability of the mature strand may influence its function and ability to enter the RISC complex to then bind to its target gene—in general, the more stable strand will be functional, and less stable strand will be degraded.

As used herein, the term “MKI67” refers to a known marker of hair follicle proliferation and morphogenesis. MKI67 is only expressed in hDPCs during active growth phases of the cell cycle (G1, S, G2, and mitosis). Ki-67 (protein encoded by MKI67) is downregulated via transcriptional control by tumor suppressor gene p53; the cell cycle is blocked through retinoblastoma protein (RB) and the p53-p21-DREAM pathway.

The term “modulate” as used herein means to regulate, alter, adapt, or adjust to a certain measure or proportion.

The term “moisturizing agent” as used herein refers to a substance that adds or restores moisture to the skin.

As used herein, the “mTOR signaling pathway” refers to a signaling pathway involving mechanistic Target Of Rapamycin (mTOR), which is an atypical serine/threonine kinase that is present in two distinct complexes. The first complex, mTOR complex 1 (mTORC1), is composed of mTOR, Raptor, GOL, and DEPTOR and is inhibited by rapamycin. It is a master growth regulator that senses and integrates diverse nutritional and environmental cues, including growth factors, energy levels, cellular stress, and amino acids. It couples these signals to the promotion of cellular growth by phosphorylating substrates that potentiate anabolic processes such as mRNA translation and lipid synthesis or limit catabolic processes such as autophagy. The small GTPase Rheb, in its GTP-bound state, is a necessary and potent stimulator of mTORC1 kinase activity, which is negatively regulated by its GTPase-activating protein (GAP), the tuberous sclerosis heterodimer TSC1/2. TSC1 and TSC2 are the tumor-suppressor genes mutated in the tumor syndrome TSC (tuberous sclerosis complex). Their gene products form a complex (the TSC1-TSC2 (hamartin-tuberin) complex), which, through its GAP activity towards the small G-protein Rheb (Ras homologue enriched in brain), is a critical negative regulator of mTORC1 (mammalian target of rapamycin complex 1) [Huang, J. Manning B D, Biochem J. (2008) 412(2): 179-90]. Most upstream inputs are funneled through Akt and TSC1/2 to regulate the nucleotide-loading state of Rheb. In contrast, amino acids signal to mTORC1 independently of the PI3K/Akt axis to promote the translocation of mTORC1 to the lysosomal surface where it can become activated upon contact with Rheb. This process is mediated by the coordinated actions of multiple complexes, including the v-ATPase, Ragulator, the Rag GTPases, and GATOR1/2. The second complex, mTOR complex 2 (mTORC2), is composed of mTOR, Rictor, GOL, Sin1, PRR5/Protor-1, and DEPTOR. mTORC2 promotes cellular survival by activating Akt, regulates cytoskeletal dynamics by activating PKCα, and controls ion transport and growth via SGK1 phosphorylation. Aberrant mTOR signaling is involved in many disease states.

As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “myeloid” as used herein means of or pertaining to bone marrow. Granulocytes and monocytes, collectively called myeloid cells, are differentiated descendants from common progenitors derived from hematopoietic stem cells in the bone marrow. Commitment to either lineage of myeloid cells is controlled by distinct transcription factors followed by terminal differentiation in response to specific colony-stimulating factors and release into the circulation. Upon pathogen invasion, myeloid cells are rapidly recruited into local tissues via various chemokine receptors, where they are activated for phagocytosis as well as secretion of inflammatory cytokines, thereby playing major roles in innate immunity [Kawamoto, H. and Minato, N. Intl J. Biochem. Cell Biol. (2004) 36 (8): 1374-1379].

As used herein, the term “nanoparticle tracking analysis” or “NTA” refers to an analytical technique for simultaneous measuring the size distribution and concentration of nanoparticles sized between 10 nm and 1500 nm in liquid suspension. It utilizes lasers to illuminate the particles, allowing a microscope-mounted camera to track their Brownian motion in real time.

The term “Next Generation Sequencing” or “NGS” as used herein refers to a method of parallel sequencing. For instance, a nucleic acid (e.g., DNA) sample is obtained and prepared into a library (meaning a collection of nucleic acid fragments from the sample). The library is prepared by fragmenting the DNA or RNA sample. Fragmentation can be performed by physical (e.g., sheared by acoustics, nebulization, centrifugal force, needles, or hydrodynamics) or enzymatic (e.g., site-specific or non-specific nucleases) methods. In some embodiments, the fragments are about 200 bp, about 20 bp, about 300 bp, or about 350 bp in length. The DNA or RNA samples are repaired at the ends (e.g., blunt-ended) and then A-tailed (e.g., an adenosine is added to the 3′ end resulting in an overhang). Adapters are ligated to each end. Adapters include sequences, such as barcodes, restriction sites, and primer sequences.

The abbreviation “NFκB” as used herein refers to a proinflammatory transcription factor that switches on multiple inflammatory genes, including cytokines, chemokines, proteases, and inhibitors of apoptosis, resulting in amplification of the inflammatory response [Barnes, PJ, (2016) Pharmacol. Rev. 68: 788-815]. The molecular pathways involved in NF-κB activation include several kinases. The classic (canonical) pathway for inflammatory stimuli and infections to activate NF-κB signaling involve the IKK (inhibitor of KB kinase) complex, which is composed of two catalytic subunits, IKK-α and IKK-β, and a regulatory subunit IKK-γ(or NFκB essential modulator [Id., citing Hayden, MS and Ghosh, S (2012) Genes Dev. 26: 203-234]. The IKK complex phosphorylates Nf-κB-bound IκBs, targeting them for degradation by the proteasome and thereby releasing NF-κB dimers that are composed of p65 and p50 subunits, which translocate to the nucleus where they bind to KB recognition sites in the promoter regions of inflammatory and immune genes, resulting in their transcriptional activation. This response depends mainly on the catalytic subunit IKK-β(also known as IKK2), which carries out IκB phosphorylation. The noncanonical (alternative) pathway involves the upstream kinase NF-κB-inducing kinase (NIK) that phosphorylates IKK-α homodimers and releases RelB and processes p100 to p52 in response to certain members of the TNF family, such as lymphotoxin-β [Id., citing Sun, SC. (2012) Immunol. Rev. 246: 125-140]. This pathway switches on different gene sets and may mediate different immune functions from the canonical pathway. Dominant-negative IKK-β inhibits most of the proinflammatory functions of NF-κB, whereas inhibiting IKK-α has a role only in response to limited stimuli and in certain cells, such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (2010) Int. Arch. Allergy Immunol. 151: 179-89].

As used herein the term “NOG” refers to noggin, which is an antagonistic inhibitor of BMP proteins, and its expression is upregulated BMP proteins in the DP cells. Negative feedback loop between BMPs and the regulator NOG in DPCs, indicating BMP signaling is tightly regulated by its negative regulator. Acts as a short-range BMP inhibitory signal that promotes placode boundary formation when overexpressed placode formation lasts longer and is disproportional in size. Serves as a marker for hair follicle-inductive features in DP cells. The Noggin-Shh signaling loop facilitates hair follicle regeneration. In noggin-knockout mice, hair follicle morphogenesis is arrested.

The term “non-coding RNA” (“ncRNA”) as used herein refers to a functional RNA molecule that is transcribed from DNA but not translated into proteins. They are classified into housekeeping and regulatory noncoding RNAs. Housekeeping ncRNAs include ribosomal RNA (rRNA, the RNA component of ribosomes), transfer RNA (tRNA, which functions as an adapter for matching amino acids to mRNA), small nuclear RNA (snRNA, which functions in RNA processing such as mRNA splicing), and small nucleolar RNAs (snoRNAs, which functions in guiding chemical modification of other RNAs). Regulatory noncoding RNAs are divided into short ncRNAs (<200 nt) and long ncRNAs (>200 nts). Short noncoding RNAs<200 nt include microRNA (miRNA), small interfering RNAs (siRNAs) and piwi-associated RNAs (piRNAs), and long noncoding RNAs (>200 nt) [Losko, M. et al. Mediators of Inflammation (2016) 1-12. 10.1155/2016/5365209].

The term “nonionic” as used herein refers to a molecule, substance, solvent, etc. that does not dissociate into ions in solution.

The term “nonscarring alopecia” refers to hair loss without permanent destruction of the hair follicle. Examples include anagen effluvium, androgenetic alopecia, chemical alopecia, folliculitis (mild), inherited disorders of the hair shaft, telogen effluvium, alopecia areata, and traumatic alopecia.

The term “non-steroidal anti-inflammatory agents” as used herein refers to a large group of agents that are aspirin-like in their action.

As used herein, the term “non-vascular plant” refers to simple, low-growing, nonflowering plants (such as a moss or liverwort) that lacks specialized conducting channels for transporting water and nutrients (e.g., xylem and phloem) and in which the photosynthetic gametophyte is the dominant stage of the life cycle. Gametophytes are plants that produce gametes through mitosis to produce a zygote. Nonvascular plants lack vascular tissues and lack true leaves, roots and stems.

The term “normal healthy subject” as used herein refers to a subject having no symptoms or other evidence of a dermatological condition.

The term “nucleic acid” as used herein refers to a polymer of nucleotides in which the 3′ position of one nucleotide sugar is linked to the 5′ position of the next by a phosphodiester bridge. In a linear nucleic acid strand, one end typically has a free 5′ phosphate group, the other a free 3′ hydroxyl group.

The term “nucleoside” as used herein refers to a compound consisting of a purine or pyridine base covalently linked to a pentose—usually ribose (in ribonucleosides) or 2-deoxyribose (in deoxyribonucleosides. Ribonucleotides containing the bases adenine, guanine, cytosine, uracil, thymine and hyoxanthine are called, respectively, adenosine, guanosine, cytidine, uridine, thymidine, and inosine. The corresponding deoxyribonucleodies are called doxyadenosine, deoxyguanosine, etc.

The term “nucleotide” as used herein refers to a nucleoside in which the sugar carries one or more phosphate groups; nucleotides are the subunits of nucleic acids.

The term “Nucleotide-binding Oligomerization Domain (NOD)-like receptors (NLRs)” as used herein refers to innate sensors that detect microbial products or cellular damage in the cytoplasm or activate signaling pathways and are expressed in cells that are routinely exposed to bacteria, such as epithelial cells, macrophages and dendritic cells. Some NLRs activate NFκB to initiate the same inflammatory responses as the TLRs, while others trigger a distinct pathway that induces cell death and the production of pro-inflammatory cytokines [Janeway's Immunobiology. 9th ed., GS, Garland Science, Taylor & Francis Group, 2017, at 96].

Subfamilies of NLRs can be distinguished based on the other protein domains they contain. For example, the NOD subfamily has an amino-terminal caspase recruitment domain (CARD), which is structurally related to the T1R death domain in MyD88, and can dimerize with CARD domains on other proteins to induce signaling. NOD proteins recognize fragments of bacterial cell wall peptidoglycans, although it is not known if they do so through direct binding or through accessory proteins [Janeway's Immunobiology. 9th ed., GS, Garland Science, Taylor & Francis Group, 2017, at 96]. NOD1 senses γ-glutamyl diaminopimelic acid (iE-DAP), a breakdown product of peptidoglycans of Gram negative and some Gram positive bacteria, whereas NOD2 recognizes muramyl dipeptide (MDP), which is present in the peptidoglycans of most bacteria. Id. Other members of the NOD family, including NLRX1 and NLRC5, have been identified, but their function is less well understood [Janeway's Immunobiology. 9th ed., GS, Garland Science, Taylor & Francis Group, 2017, at 96-98]

When NOD1 or NOD2 recognizes its ligand, it recruits the CARD-containing serine-threonine kinase RIP2 (also known as RICK and RIPK2), which associates with the E3 ligases cIAPI, CIAP2, and XIAP, whose activity generates a polyubiquitin scaffold, which recruits TAK1 and IKK and results in activation of NFκB. NFκB then induces the expression of genes for inflammatory cytokines and for enzymes involved in the production of NO [Janeway's Immunobiology. 9th ed., GS, Garland Science, Taylor & Francis Group, 2017, at 97].

Macrophages and dendritic cells express both TLRs and NOD1 and NOD2 and are activated by both pathways. In epithelial cells, NOD1 may also function as a systemic activator of innate immunity. NOD2 is strongly expressed in the Paneth cells of the gut where it regulates the expression of potent anti-microbial peptides such as the α- and β-defensins [Janeway's Immunobiology. 9th ed., GS, Garland Science, Taylor & Francis Group, 2017, at 97].

Other members of the NOD family, including NLRX1 and NLRC5, have been identified, but their function is less well understood [Janeway's Immunobiology. 9th ed., GS, Garland Science, Taylor & Francis Group, 2017, at 96-98].

The NLRP family, another subfamily of NLR proteins, has a pyrin domain in place of the CARD domain at their amino termini. Humans have 14 NLR proteins containing pyrin domains, of which NLRP3 (also known as NAPL3 or cryopyrin) is the best characterized. NLRP3 resides in an inactive form in the cytoplasm, where its leucine rich repeat (LRR) domains are thought to bind the head-shock chaperone protein HSP90 and the co-chaperone SGT1. NRLP3 signaling is induced by reduced intracellular potassium, the generation of reactive oxygen species, or the disruption of lysosomes by particulate or crystalline matter. For example, death of nearby cells can release ATP into the extracellular space, which would activate the purinergic receptor P2X7, which is a potassium channel, and allow potassium ion efflux. A model proposed for ROS-induced NLRP3 activation involves intermediate oxidation of sensor proteins collectively called thioredoxin (TRX). Normally TRX proteins are bound to thioredoxin-interacting protein (TXNIP). Oxidation of TRX by ROS causes dissociation of TXNIP from TRX. The free TXNIP may then displace HSP90 and SGT1 from NLRP3, again causing its activation. In both cases, NLRP3 activation involves aggregation of multiple monomers via their leucine-rich repeat (LRR) and NOD domains to induce signaling. Phagocytosis of particulate matter (e.g. the adjuvant alum), may lead to the rupture of lysosomes and release of the active protease cathepsin B, which can activate NLRP3 [Janeway's Immunobiology. 9th ed., GS, Garland Science, Taylor & Francis Group, 2017, at 98-99].

NLR signaling, as exemplified by NLRP3, leads to the generation of pro-inflammatory cytokines and to cell death through formation of an inflammasome, a multiprotein complex. Activation of the inflammasome proceeds in several stages. Aggregation of NLRP molecules triggers autocleavage of procaspase I, which releases active caspase 1-Aggregation of LRR domains of several NLRP3 molecules, or other NLRP molecules by a specific trigger or recognition event, which induces the pyrin domains of NLRP3 to interact with pyrin domains of ASC (also called PYCARD), an adaptor protein composed of an amino terminal pyrin domain and a carboxy terminal CARD domain, which further drives the formation of a polymeric ASC filament, with the pyrin domains in the center and the CARD domains facing outward; the CARD domains then interact with CARD domains of the inactive protease pro-caspase 1, initiating its CARD-dependent polymerization into discrete caspase 1 filaments. Active caspase 1 then carries out ATP-dependent proteolytic processing of proinflammatory cytokines, particularly 1L-1β and IL-18, into their active forms, and induces a form of cell death (pyroptosis) associated with inflammation because of the release of these pro-inflammatory cytokines upon cell rupture [Janeway's Immunobiology. 9th ed., GS, Garland Science, Taylor & Francis Group, 2017, at 99-100].

A priming step, which can result from TLR signaling, must first occur in which cells inducer and translate the mRNAs that encode the pro-forms of IL-1, IL-18 or other cytokines for inflammasome activation to produce inflammatory cytokines. For example, the TLR-3 agonist poly I:C can be used experimentally to prime cells for triggering of the inflammasome [Janeways Immunobiology. 9th ed., GS, Garland Science, Taylor & Francis Group, 2017, at 100].

Inflammasome activation also can involve proteins of the PYHIN family, which have an H inversion (HIN) domain in place of an LRR domain. There are four PYIN proteins in humans. Id. at 100. A noncanonical inflammasome (caspase I-independent) pathway uses the protease caspase 11, which therefore is both a sensor and an effector molecule, to detect intracellular LPS [Janeway's Immunobiology. 9th ed., GS, Garland Science, Taylor & Francis Group, 2017, at 101].

Besides activating effector functions and cytokine production, another outcome of the activation of innate sensing pathways is the induction of co-stimulatory molecules on tissue dendritic cells and macrophages. B7.1 (CD80) and B7.2 (CD86), for example, which are induced on macrophages and tissue dendritic cells by innate sensors such as TLRs in response to pathogenic recognition, are recognized by specific co-stimulatory receptors expressed by cells of the adaptive immune response, particularly CD4 T cells, and their activation by B7 is an important step in activating adaptive immune responses [Janeway's Immunobiology. 9th ed., GS, Garland Science, Taylor & Francis Group, 2017, at 105].

The term “nutraceutical,” as used herein refers to a non-toxic dietary compound with extra health benefits in addition to the basic nutritional value found in foods. Nutraceuticals can be of either plant or animal origin. According to FDA regulations, most nutraceuticals would be categorized as “dietary supplements”. These are extracts, concentrates, metabolites, constituents or combinations of vitamins, minerals, botanicals, herbs, amino acids or dietary substances “for use by man to supplement the diet by increasing the total dietary intake.” In order to maintain classification as a dietary supplement (and avoid the FDA's strict drug approval process), nutraceutical labeling must maintain that the nutraceutical is not intended to diagnose, treat, cure or prevent any disease.

The term “organ” as used herein refers to a differentiated structure consisting of cells and tissues and performing some specific function in an organism.

The term “overexpressed” as used herein refers to increased quantity of a gene or gene product relative to a quantity of the gene or gene product under normal conditions.

The term “oxidative stress” as used herein refers to a condition where the levels of ROS significantly overwhelm the capacity of antioxidant defenses, leading to potential damage in a biological system. An oxidative stress condition can be caused by either increased ROS formation or decreased activity of antioxidants or both. Under certain circumstances, small transient increases in ROS levels can be employed as a signaling mechanism, leading to physiological cellular responses [Li, R., et al. React. Oxyg. Species (Apex) (2016) 1 (1): 9-21].

As used herein, the term “paracrine signaling” refers to short range cell-cell communication via secreted signal molecules that act on adjacent cells.

The term “particle” as used herein refers to an extremely small constituent, e.g., microparticles (particles in the micrometer size range), nanoparticles (particles in the nanometer size range), etc.

The term “PAMPs” is an abbreviation for pathogen-associated molecular patterns. PAMPS are structural patterns present in components or products common to a wide variety of microbes but not host cells. PAMPS are ligands for pattern recognition molecules (PRMs).

The term “pattern recognition molecules” or “PRMs” as used herein refer to proteins recognizing PAMPs. Soluble PRMs include the collectins, acute phase proteins and NOD proteins. Membrane-bound PRMs are pattern recognition receptors.

The term “pattern recognition receptors” or “PRRs” refers to widely distributed membrane bound PRMs fixed in either the plasma membrane of a cell or in the membranes of its endocytic vesicles. The term PRRs includes toll-like receptors (TLRs) and scavenger receptors. Engagement of PRRs induces pro-inflammatory cytokines.

“Percutaneous absorption” is the absorption of substances from outside the skin to positions beneath the skin, including into the blood stream. The epidermis of human skin is highly relevant to absorption rates. Passage through the stratum corneum marks the rate-limiting step for percutaneous absorption. The major steps involved in percutaneous absorption of, for example, a drug, include the establishment of a concentration gradient, which provides a driving force for drug movement across the skin, the release of drug from the vehicle into the skin-partition coefficient and drug diffusion across the layers of the skin-diffusion coefficient. The relationship of these factors to one another is summarized by the following equation:

J=Cᵥₑₕ×KₘD/x [Formula 1]
where J=rate of absorption
Cᵥₑₕ=concentration of drug in vehicle
Kₘ partition coefficient, which describes how a solute is distributed between two immiscle solvents);
D=diffusion coefficient, which indicates speed of diffusion and is dependent on molecule size, and other properties of the solute as well as temperature and pressure;
X=path length/thickness.

There are many factors which affect the rate of percutaneous absorption of a substance. Primarily they are as follows: (i) Concentration. The more concentrated the substance, the greater the absorption rate; (ii) Size of skin surface area to which the drug is applied. The wider the contact area of the skin to which the substance is applied, the greater the absorption rate; (iii) Anatomical site of application. Skin varies in thickness in different areas of the body. A thicker and more intact stratum corneum decreases the rate of absorbency of a substance. The stratum corneum of the facial area is much thinner than, for example, the skin of the palms of the hands. The facial skin's construction and the thinness of the stratum corneum provide an area of the body that is optimized for percutaneous absorption to allow delivery of active agents both locally and systemically through the body; (iv) Hydration. Hydration (meaning increasing the water content of the skin) causes the stratum corneum to swell which increases permeability; (v) Temperature—increased skin temperature increases permeability; and (vi) The composition of the compound and of the vehicle also determines the absorbency of a substance. Most substances applied topically are incorporated into bases or vehicles. The vehicle chosen for a topical application will greatly influence absorption and may itself have a beneficial effect on the skin. Ideally, a vehicle that has use in the present invention is easy to apply and remove, nonirritating, and cosmetically pleasing. In addition, the active must be stable in the chosen vehicle and must be released readily. Factors that determine the choice of vehicle and the transfer rate across the skin are the substance's partition coefficient, molecular weight and water solubility. The protein portion of the stratum corneum is most permeable to water soluble substances and the liquid portion of the stratum corneum is most permeable to lipid soluble substances. It follows that substances having both liquid and aqueous solubility can traverse the stratum corneum more readily. See Dermal Exposure Assessment: Principles and Applications, EPA/600/8-91/011b, January 1992, Interim Report—Exposure Assessment Group, Office of Health and Environmental Assessment, U.S. Environmental Protection Agency, Washington, D.C. 20460.

The term “peptide” is used herein to refer to two or more amino acids joined by a peptide bond. Solid phase peptide synthesis may be accomplished by techniques familiar to those in the art and provided, for example, in Stewart and Young, 1984, Solid Phase Synthesis, Second Edition, Pierce Chemical Co., Rockford, III; Fields and Noble, 1990, Int. J. Pept. Protein Res. 35:161-214 or using automated synthesizers.

The term “peptidomimetic” as used herein refers to a small protein-like chain designed to mimic a peptide. A peptidomimetic typically arises from modification of an existing peptide in order to alter the molecule's properties.

The term “pharmaceutical composition” is used herein to refer to a composition that is employed to prevent, reduce in intensity, cure or otherwise treat a target condition or disease.

The term “pharmaceutically acceptable,” is used to refer to the carrier, diluent or excipient being compatible with the other ingredients of the formulation or composition and not deleterious to the recipient thereof. The carrier must be of sufficiently high purity and of sufficiently low toxicity to render it suitable for administration to the subject being treated. The carrier should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use in animals, and more particularly in humans.

The term “pharmaceutically acceptable carrier” refers to any substantially non-toxic carrier conventionally useable for administration of pharmaceuticals in which the isolated extracellular vesicles will remain stable and bioavailable. The pharmaceutically acceptable carrier must be of sufficiently high purity and of sufficiently low toxicity to render it suitable for administration to the mammal being treated. The pharmaceutically acceptable carrier may be liquid or solid and is selected, with the planned manner of administration in mind, to provide for the desired bulk, consistency, etc., when combined with an active agent and other components of a given composition.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio.

The term “phenotype” refers to the observable physical properties of an organism; these include the organism's appearance, development, and behavior.

The term “photoaging” as used herein refers to the cumulative detrimental effects (such as wrinkles or dark spots) on skin that result from long-term exposure to sunlight, e.g., ultraviolet light.

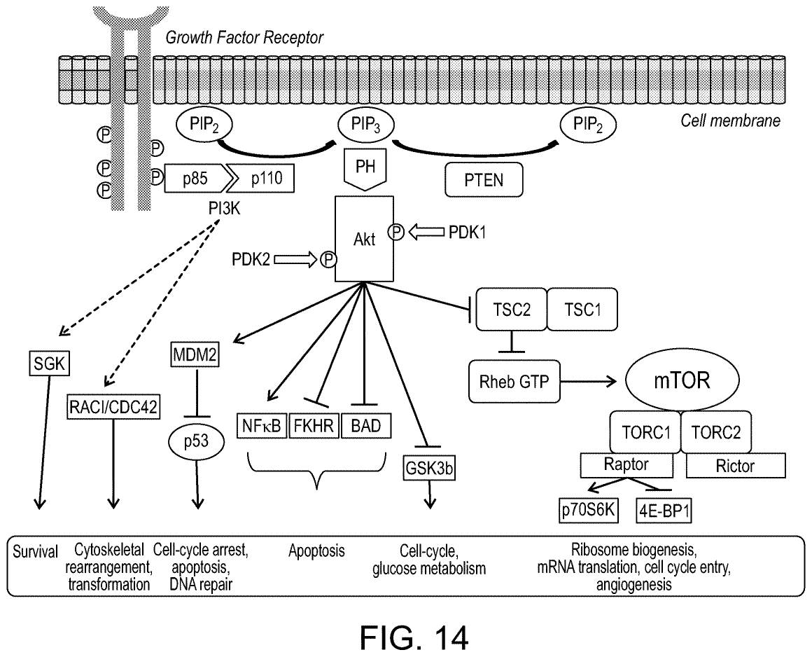

As used herein, the “PI3K/Akt/mTOR signaling pathways” refers to a signaling pathway that is crucial to many aspects of cell growth and survival [Porta, C., et al. Frontiers in Oncology (2014) doi.10.3389/fpmc.2014.00064]. A schematic of the PI3K/Akt/mTOR pathway is shown in FIG. 14 (Taken from Porta, et al. Front. Oncol. (2014) 4: art. 64). PI3Ks constitute a lipid kinase family characterized by the capability to phosphorylate inositol ring 3′-OH group in inositol phospholipids [Id., citing Fruman, D A., et al. Annu. Rev. Biochem. (1998) 67: 481-507]. Class I PI3Ks are heterodimers composed of a catalytic (CAT) subunit (i.e., p110) and an adaptor/regulatory subunit (i.e., p85). This class is further divided into two subclasses: subclass IA (PI3Kα, β, and δ), which is activated by receptors with protein tyrosine kinase activity, and subclass IB (PI3Kγ), which is activated by receptors coupled with G proteins (Id., citing Fruman, D A., et al. Phosphoinositide kinases. Annu. Rev. Biochem. (1998) 67: 481-507). Activation of growth factor receptor protein tyrosine kinases results in autophosphorylation on tyrosine residues. PI3K is then recruited to the membrane by directly binding to phosphotyrosine consensus residues of growth factor receptors or adaptors through one of the two SH2 domains in the adaptor subunit. This leads to allosteric activation of the CAT subunit. PI3K activation leads to the production of the second messenger phosphatidylinositol-4,4-bisphosphate (PI3,4,5-P3) from the substrate phosphatidylinositol-4,4-bisphosphate (PI-4,5-P2). PI3,4,5-P3 then recruits a subset of signaling proteins with pleckstrin homology (PH) domains to the membrane, including protein serine/threonine kinase-3′-phosphoinositide-dependent kinase I (PDK1) and Akt/protein kinase B (PKB) [Id., citing Fruman, D A., et al., Phosphoinositide kinases. Annu. Rev. Biochem. (1998) 67: 481-507, Fresno-Vara, J A., et al., PI3K/Akt signaling pathway and cancer. Cancer Treat. Rev. (2004) 30: 193-204]. Akt/PKB, on its own, regulates several cell processes involved in cell survival and cell cycle progression. Akt (also known as protein kinase B) is a 60 kDa serine/threonine kinase. It is activated in response to stimulation of tyrosine kinase receptors such as platelet-derived growth factor (PDGF), insulin-like growth factor, and nerve growth factor [Shimamura, H, et al. J. Am. Soc. Nephrol. (2003) 14: 1427-1434; Datta K., et al., Mol Cell Biol (1995) 15: 2304-2310; Kulik G., et al. Mol Cell Biol (1997) 17: 1595-1606; Yao R and Cooper G M, Science (1995) 267: 2003-2006). Stimulation of Akt has been shown to be dependent on phosphatidylinositol 3-kinase (PI3-kinase) activity (Fruman D A, et al. Annu Rev Biochem (1998) 67: 481-507; Choudhury G, et al. Am J Physiol (1997) 273: F931-938, Franke T F., et al. Cell (1995) 81: 727-736; Franke T F., et al. Cell (1997) 88: 435-437]. Akt has been shown to act as a mediator of survival signals that protect cells from apoptosis in multiple cell lines [Brunet A., et al. Cell (1999) 96: 857-868; Downward J, Curr Opin Cell Biol 10: 262-267, 1998). For example, phosphorylation of the pro-apoptotic Bad protein by Akt was found to decrease apoptosis by preventing Bad from binding to the anti-apoptotic protein Bcl-XL [Dudek H, et al. Science (1997) 275: 661-665; Datta S R, et al. Cell (1997) 91: 231-241]. Akt was also shown to promote cell survival by activating NF-kB [Cardone M H, et al. Science (1998) 282: 1318-1321; Khwaja A, Nature (1999) 401: 33-34] and inhibiting the activity of the cell death protease caspase-9 [Kennedy S G., et al. Mol Cell Biol (1999) 19: 5800-5810]. PI3K also acts as a branch point in response to TGF-β, leading to activation of PAK2/c-Abl, which stimulates collagen gene expression in normal fibroblasts, and induces fibroblast proliferation, thereby increasing the number of myofibroblast precursors [He, W and Dai, C. Curr. Pathobiol. Rep. (2015) 3: 183-92, citing Wilkes, M C and Leof, EB. J. Biol. Chem. (2006) 281: 27846-27854]. PAK2/c-Abl promotes fibrosis through its downstream mediators, including PKC6/Fli-1 and early growth response (Egr)-1, -2, and -3 [Id., citing Tsou, P S., et al. A. J. Physiol. Cell Physiol. (2014) 307: C2-C13; Bhattacharyya, S., et al. J. Pathol. (2013) 229: 286-297; Fang, F, et al. Am. J. Pathol. (2013) 183: 1197-1208].

As used herein, the term “piwi-interacting RNA” or “piRNA” refers to the largest class of small non-coding RNA molecules (typically 24-31 nucleotides long). They associate with PIWI proteins to form silencing complexes that protect germline genome integrity by suppressing transposable elements at both transcriptional and post-transcriptional levels.

The term “polymerase chain reaction” or “PCR” is a laboratory technique used to amplify small segments of DNA sequences. Conventional PCR is a thermal cycling process in which nucleic acid molecules are amplified through repeated cycles of denaturation, primer annealing, and extension using a DNA polymerase

The term “polypeptide” is used herein in its broadest sense to refer to a sequence of subunit amino acids, amino acid analogs or peptidomimetics, wherein the subunits are linked by peptide bonds. Polypeptides can be chemically synthesized or recombinantly expressed. Synthetic polypeptides prepared using the techniques of solid phase, liquid phase, or peptide condensation techniques, or any combination thereof, can include natural and unnatural amino acids. Amino acids used for peptide synthesis may be standard Boc (N-α-ammo protected N-α-t-butyloxycarbonyl) amino acid resin with the standard deprotecting, neutralization, coupling and wash protocols of the original solid phase procedure of Merifield (1963, J. Am. Chem. Soc. 85:2149-2154), or the base-labile N-α-amino protected 9-fluorenylmethoxycarbonyl (Fmoc) amino acids first described by Carpino and Han (1972, J. Org. Chem. 37:3403-3409). The polypeptides of the invention may comprise D-amino acids (which are resistant to L-amino acid-specific proteases in vivo), a combination of D- and L-amino acids, and various “designer” amino acids (e.g., β-methyl amino acids, C-α-methyl amino acids, and N-α-methyl amino acids, etc.) to convey special properties. Synthetic amino acids include ornithine for lysine, and norleucine for leucine or isoleucine. In addition, the polypeptides can have peptidomimetic bonds, such as ester bonds, to prepare peptides with novel properties. For example, a peptide may be generated that incorporates a reduced peptide bond, i.e., R1-CH2-NH—R2, where Ri and R2 are amino acid residues or sequences. A reduced peptide bond may be introduced as a dipeptide subunit. Such a polypeptide would be resistant to protease activity and would possess an extended half-live in vivo. Accordingly, these terms also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids.

The terms “polypeptide”, “peptide” and “protein” also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. In some embodiments, the peptide is of any length or size. Use herein of the terms “peptide”, “peptides”, “polypeptide”, “peptidomimetic” or “protein” should be taken to include reference to “derivatives” of such compounds, unless the context requires otherwise, and to include “prodrugs.”

The term “potency” and its various grammatical forms as used herein, refers to power or strength of a formulation to produce a defined effect.

As used herein, the term “primer” refers to a short, single stranded nucleic acid that serves as a necessary starting point for DNA synthesis. Primers provide a free 3′-OH group that DNA polymerases need to begin adding nucleotides, acting as a foundation for building new DNA strands during replication of PCR.

As used herein, “primer dimer” refers to small, undesired byproducts of PCR formed when primers hybridize to each other, rather than the target DNA. These artifacts compete for reagents, reducing amplification efficiency. They often appear as fuzzy, low-molecular weight bands at the bottom of a gel, or as distinct, low-temperature peaks in melting curve analyses.

The term “priming” as used herein refers to the first encounter with a given antigen, which generates a primary adaptive immune response. The term “unprimed cells” (also referred to as virgin, naïve, or inexperienced cells) as used herein refers to T cells and B cells that have generated an antigen receptor (TCR for T cells, BCR for B cells) of a particular specificity, but have never encountered the antigen. For example, before helper T cells and B cells can interact to produce specific antibody, the antigen-specific T cell precursors must be primed.

Priming involves several steps: antigen uptake, processing, and cell surface expression bound to class II MHC molecules by an antigen presenting cell, recirculation and antigen-specific trapping of helper T cell precursors in lymphoid tissue, and T cell proliferation and differentiation [Janeway, C A, Jr., “The priming of helper T cells, Semin. Immunol. (1989) 1(1): 13-20]. Helper T cells express CD4, but not all CD4 T cells are helper cells. Id. The signals required for clonal expansion of helper T cells differ from those required by other CD4 T cells. The critical antigen-presenting cell for helper T cell priming appears to be a macrophage; and the critical second signal for helper T cell growth is the macrophage product interleukin 1 (IL-1). Id. If the primed T cells and/or B cells receive a second, co-stimulatory signal, they become activated T cells or B cells.

The term “protein” as used herein refers to a large complex molecule or polypeptide composed of amino acids.

The term “protective” as used herein is used in the broadest pharmacological sense to mean any agent that isolates the exposed surface of the skin or other membrane from harmful or annoying stimuli.

The term “purification” and its various grammatical forms as used herein refers to the process of isolating or freeing from foreign, extraneous, or objectionable elements.

The term “quantitative PCR” (or “qPCR”), also called “real time-PCR” or “quantitative real-time PCR” refers to a polymerase chain reaction-based technique that couples amplification of a target DNA sequence with quantification of the concentration of that DNA species in the reaction.

The term “Reactive Oxygen Species or “ROS” as used herein, such as free radicals and peroxides, refers to a class of molecules that are derived from the metabolism of oxygen and exist inherently in all aerobic organisms. It is a collective term to include superoxide (O2·-), hydrogen peroxide (H₂O₂), hydroxyl radical (OH·), singlet oxygen (1O2), peroxyl radical (LOO·), alkoxyl radical (LO·), lipid hydroperoxide (LOOH), peroxynitrite (ONOO—), hypochlorous acid (HOCI), and ozone (O3), among others [Li, R., et al. React. Oxyg. Species (Apex) (2016) 1 (1): 9-21]. The term “oxygen radicals” as used herein refers to any oxygen species that carries an unpaired electron (except free oxygen). The transfer of electrons to oxygen also may lead to the production of toxic free radical species. The best documented of these is the superoxide radical. Oxygen radicals, such as the hydroxyl radical (OH−) and the superoxide ion (O2-) are very powerful oxidizing agents that cause structural damage to proteins, lipids and nucleic acids. The free radical superoxide anion, a product of normal cellular metabolism, is produced mainly in mitochondria because of incomplete reduction of oxygen. The superoxide radical, although unreactive compared with many other radicals, may be converted by biological systems into other more reactive species, such as peroxyl (ROO—), alkoxyl (RO—) and hydroxyl (OH—) radicals.

Many abnormal physiological and metabolic processes in organisms produce biological oxidants such as superoxide radicals (O₂·—), hydroxyl radicals (·OH), and hydrogen peroxide (H₂O₂), which could endow several different biological targets with reactivity and instability, such as DNA, proteins, and membrane lipids [Zheng, M., et al. Antioxidants (2023) 12: 1675, citing Taverne, Y. J. H. J., et al. Oxidative Med. Cell Longev. (2013) 2013: 862423]. In the oxidative stress state, biological oxidants lead to an imbalance between oxidation and anti-oxidation in vivo, cellular oxidants activate various apoptosis inducers through certain transcription factors or directly lead to lipid peroxidation, protein and DNA damage, and enzyme expression disorders. Physiological production of superoxide radical anion and hydrogen peroxide is essential for redox signaling in cells, but their excessive generation could lead to oxidative stress and oxidative modifications of biomolecules and lipid peroxidation, which could be related to pathophysiological processes.

Aerobic organisms possess antioxidant defense systems that deal with reactive oxygen species (ROS) produced as a consequence of aerobic respiration and substrate oxidation. In the process of normal cellular metabolism, oxygen undergoes a series of univalent reductions, leading sequentially to the production of O₂·-, hydrogen peroxide (H₂O₂), and H₂O. Potential enzymatic source of ROS includes components of the mitochondrial electron transport chain, xanthine oxidase, the cytochrome p450 monooxygenases, lipoxygenase, nitric oxide synthase (NOS), and the NADPH oxidase (Fukai, T. and Ushio-Fukai, Antioxidants & Redox Signaling (2011) 15 (6): 1583-1606, citing Guzik, T J and Harrison, DG. Drug Discov. Today (2006) 11: 524-533). Superoxide anion is dismutated by superoxide dismutases (SODs) to H₂O₂ that is catalyzed to H₂O by catalase, peroxiredoxins (Prxs), or glutathione peroxidases (GPx) Low levels of either intracellular or extracellular ROS (e.g., superoxide and H₂O₂) are indispensable in many biochemical processes, including intracellular signaling, defense against microorganisms, and cell function (Id., citing Go, Y M and Jones, D P. Free Radic. Biol. Med. (2011) 50 (4): 495-509; Lassegrue, B. and Griendling, KK. (2010) Arterioscler. Thromb. Vasc. Biol. (2010) 30: 653-61; Ushio-Fukai, M. Antiox. Redoc. Signal. (2009) 11: 1289-1299). In contrast, high dose and/or inadequate removal of ROS, especially superoxide anion, results in oxidative stress, which has been implicated in the pathogenesis of many cardiovascular diseases, including hypercholesterolemia, atherosclerosis, hypertension, diabetes, and heart failure. ROS also represent a component of the innate immune system; they are not only involved in the respiratory burst of neutrophils but also signal inflammatory cell chemotaxis into sites of inflammation [Id., citing Bogdan, C., et al. Curr. Opin. Immunol. (2000) 12: 64-76].

Nitric oxide (NO), which has anti-inflammatory and anticoagulant properties as well as a vasodilator effect, can be rapidly inactivated by reaction with O2·- leading to the production of the strong oxidant peroxynitrite (ONOO—). This reaction is important in common conditions leading to endothelial and mitochondrial dysfunction, including hypercholesterolemia, hypertension, diabetes, and aging, in which vascular production of O2·- is increased (Id., citing Guzik T J. Harrison D G. Drug Discov. Today. (2006) 11:524-533; Madamanchi N R. and Runge M S. Circ Res. (2007) 100: 460-473).

The term “redox signaling” as used herein refers to a physiological process, where ROS act as second messengers to mediate responses that are required for proper function and survival of the cell. On the other hand, redox modulation (or redox regulation) refers to a process wherein ROS alter the activity or function of the redox-sensitive molecular targets, including signaling proteins and metabolic enzymes, leading to either physiological or pathophysiological responses. When pathophysiological responses occur, it is also known as oxidative stress [Li, R., et al. React. Oxyg. Species (Apex) (2016) 1 (1): 9-21].

As used herein, the term “reference allele” refers to the sequence of one or more nucleotides at a chromosomal locus that is either the predominant allele represented at that chromosomal locus within the population of the species (e.g., the “wild-type” sequence), or an allele that is predefined within a reference genome for the species.

As used herein, the term “reference genome” refers to a standardized representative assembled nucleic acid sequence (e.g., FASTA format) used as a template to map, align, and analyze NGS data.

The term “rejuvenate” and its various grammatical forms as used herein refer to making young or youthful again.

The term “renewal” or “self-renewal” as used herein, refers to the process by which a stem cell divides to generate one (asymmetric division) or two (symmetric division) daughter cells having development potential indistinguishable from the mother cell. Self-renewal involves both proliferation and the maintenance of an undifferentiated state.

The term “repair” as used herein as a noun refers to any correction, reinforcement, reconditioning, remedy, making up for, making sound, renewal, mending, patching, or the like that restores function. When used as a verb, it means to correct, to reinforce, to recondition, to remedy, to make up for, to make sound, to renew, to mend, to patch or to otherwise restore function.

As used herein, the term “resistive pulse sensing” refers to a precise, Coulter-principle-based technology for quantifying nanoparticles, particularly small EVs. It measures individual EVs as they pass through a nanopore, providing accurate, particle-by-particle size and concentration measurements, and in some cases provides an added capacity to analyze surface markers (Song Y, Micromachines (Basel). 2017 Jun. 25; 8(7):204).

The term “restriction enzyme” as used herein refers to an enzyme produced by bacteria that recognizes a short sequence in a nucleic acid molecule and cleaves the nucleic acid molecule at or near that sequence, resulting in a double-stranded or single-stranded break. The term “restriction enzyme recognition site” or “restriction site” as used herein is a short specific sequence of base pairs of DNA recognized by each restriction enzyme.

The term “resuscitate” as used herein refers to being restored to life or being revived.

The term “reverse” as used herein refers to turning backward or in an opposite direction.

As used herein, the term “reverse transcription” or grammatical variations thereof, refers to the process of copying the nucleotide sequence of an RNA molecule into a DNA molecule. Reverse transcription can be performed experimentally by reacting an RNA template with an RNA-dependent DNA polymerase (also known as a reverse transcriptase) under well-known conditions. A reverse transcriptase is a DNA polymerase that transcribes single-stranded RNA into single stranded DNA. Depending on the polymerase used, the reverse transcriptase can also have RNase H activity for subsequent degradation of the RNA template.

As used herein, the term “ribosomal RNA” or “rRNA” refers to a type of noncoding RNA that serves as the primary structural and functional component of ribosomes.

The term “RISC” or “RNA-induced silencing complex”, as used herein, refers to a multiprotein complex that incorporates one strand of a small interfering RNA (siRNA) or micro RNA (miRNA). RISC uses the siRNA or miRNA as a template for recognizing complementary mRNA. When it finds a complementary strand, it activates RNase and cleaves the RNA. This process is important both in gene regulation by microRNAs and in defense against viral infections, which often use double-stranded RNA as an infectious vector.

The term “RNA” or “ribonucleic acid” as used herein refers to a nucleic acid consisting of ribonucleotides, each of which contains one of the bases adenine, guanine, cytosine or uracil, or, in some RNAs, a modified form of one of these bases. RNA molecules usually are single-stranded, but an RNA strand can form a duplex with a complementary strand of RNA or DNA.

The term “RNA molecule” or “ribonucleic acid molecule,” as used herein, refers to a linear, single-stranded polymer composed of ribose nucleotides that is synthesized by transcription of DNA or by copying of RNA. It encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties

The term “rubefacient” as used herein refers to an agent that induces hyperemia, wherein hyperemia means an increased amount of blood in a body part or organ. Rubefaction, which is induced by rubefacients, results from increased circulation to an injured area and is accompanied by a feeling of comfort, warmth, itching and hyperesthesia.

The term “scarring alopecia”, also known as “cicatricial alopecia”, refers to a collection of hair loss disorders that may be diagnosed in up to 3% of hair loss patients. It occurs worldwide in otherwise healthy men and women of all ages. While there are many forms of scarring alopecia, the common theme is a potentially permanent and irreversible destruction of hair follicles and their replacement with scar tissue. Examples include bullous diseases, chemical alopecia, discoid lupus erythematosus, folliculitis (severe), lichen planopilaris, dissecting cellulitis, and tumors.

The term “semaphorins” or “Semas” as used herein refer to a large and diverse family of proteins that are divided into 8 classes based on structural features and distribution among different phyla. Class 1 and 2 Semas are found only in invertebrates, while class 3-7 are found only in vertebrates (with the one exception being Sema-5c, which is also found in invertebrates). Class V Semas are found in viruses. Class 1, 4, 5, and 6 members are transmembrane, class 2, 3 and V members are secreted, and class 7 members are glycosylphosphatidylinositol (GPI)-linked. In addition, class 4, 5 and 7 members, and possibly others, are cleaved and released extracellularly. Plexin receptors, the predominant receptors for Semas, are grouped into 4 classes (A-D) and each plexin receptor class interacts with a particular Sema class or classes to mediate signaling. A number of other membrane-associated receptors and co-receptors are also important for Sema signal transduction. These proteins directly bind Semas and initiate signaling (e.g., integrins), act as ligand binding co-receptors (e.g., Npn1,2), and/or work as part of multimeric receptor complexes (e.g., RTKs). The hallmark of the Sema protein family is the sema domain, an approximately 500 amino acid extracellular domain (2). the sema domain, is present as a single copy located at the N-terminus of Sema proteins and is essential for Sema signaling [Alto, L T and Terman, JR, Methods Mol Biol. (2017) 1493: 1-25]. Plexin B1, the Sema 4D receptor, is a tumor suppressor protein for melanoma, in part through inhibition of the oncogenic c-Met tyrosine kinase receptor. Sema4D is a protective paracrine factor for normal human melanocyte survival in response to ultraviolet irradiation; it stimulates proliferation and regulates the activity of the c-Met receptor. C-Met receptor signaling stimulates melanocyte migration, in part through down-regulation of the cell adhesion molecule E-cadherin [Soong, J. et al. J. Invest. Dermatol. (2012) 132 (4): 1230-1238].

As used herein, the term “SFRP1 refers to Secreted Frizzled Related Protein 1 (SFRP1) wherein its expression is significantly increased in hair follicles and DPCs of androgenetic alopecia (AGA) subjects compared to normal hair subjects. Exogenous SFRP1 treatment of DPCs causes G1 cell cycle arrest. Conversely, SFRP1 inhibition leads to enhanced hair shaft production and inhibited spontaneous catagen, promoting hair growth.

The term “signature” as used herein refers to a specific and complex combination of biomarkers that reflect a biological state.

As used here, the term “size exclusion chromatography” refers to a chromatographic technique wherein a biological sample is loaded onto a column packed with porous beads, which separates components of the sample based on size.

The term “skin clarity” as used herein refers to a smooth, even skin texture that's free of lumps and bumps.

The term “skin luminosity” as used herein refers to a measure of the intensity of the light reflected from the skin's surface as it bounces back into the observer's eye. The amount of light reflected from the skin is dependent on how smooth the skin is.

The term “skin rejuvenation” as used herein refers to treatments that aim to restore skin from any damage. Skin damage can be a result of sun exposure, an underlying health condition, or a normal sign of aging.

The term “skin texture” or “skin roughness” as used herein refers to condition of the surface of the skin. Young healthy skin is well hydrated, and firm with a smooth, soft texture and luminosity. In aged skin, cell turnover slows, resulting in a buildup of dead skin cells which contributes to the appearance of dull skin and creates clogged pores.

As used herein, the term “small interfering RNA” or “siRNA” refers to a class of double-stranded, non-coding RNA molecules, typically 20-25 base pairs long, that regulate gene expression through the RNA interference (RNAi) pathway.

As used herein, the term “small nuclear RNA” or “snRNA” refers to a class of short (~150 nucleotides), non-coding RNA molecules found within the eukaryotic nucleus. They associate with proteins to form small nuclear ribonucleoproteins (snRNPs or “snurps”), which are essential components of the spliceosome machinery that removes introns and splices exons in pre-messenger RNA (mRNA).

The terms “soluble” and “solubility” refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent. Among the acceptable vehicles and solvents that may be employed are water, Ringer's solution, and isotonic sodium chloride solution.

A “solution” generally is considered as a homogeneous mixture of two or more substances. It is frequently, though not necessarily, a liquid. In a solution, the molecules of the solute (or dissolved substance) are uniformly distributed among those of the solvent.

The term “solvate” as used herein refers to a complex formed by the attachment of solvent molecules to that of a solute.

The term “solvent” as used herein refers to a substance capable of dissolving another substance (termed a “solute”) to form a uniformly dispersed mixture (solution).

The term “SOX2”, as used herein refers to a transcription factor important in progenitor cells and stem cells. SOX2 has been identified to have high expression in dermal papilla cells while not being expressed in hair follicle stem cells. Hair growth could be affected due to the transcriptional control of SOX2 on dermal papilla cells. SOX2 does act as a direct transcriptional regulator in dermal papilla cells in the early growth and development of the hair follicles. SOX2 also has an effect on BMP signal regulation in the HF. Specifically, when SOX2 is downregulated, both BMP6 and WIF1 are upregulated; these proteins inhibit hair growth.

As used herein, the term “STAT5A” refers to Signal transducer and activator of transcription 5A and is upregulated in DPCs. STAT5 activation is crucial for the transition from telogen to anagen in the hair growth cycle. Deletion of STAT5 in DPCs delays the hair follicle's entry into the anagen phase.

As used herein, the term “STAT6” refers to Signal Transducer and Activator of Transcription 6 (STAT6) and plays an important role in IL4-mediated signaling. In a study investigating the effects of 3-Deoxysappanchalcone (3-DSC) on hair growth, it was found that 3-DSC promoted DPC proliferation via inhibition of STAT6. Normally, STAT6 is activated in quiescent hair follicles (hair follicles undergoing telogen). Other studies have found that plant extracts increase hair growth and promote DPC proliferation.

The term “stem cells” refers to undifferentiated cells having high proliferative potential with the ability to self-renew that can generate daughter cells that can undergo terminal differentiation into more than one distinct cell phenotype.

The term “stirred tank bioreactor”, as used herein refers to a vessel configured for culturing cells or biological materials in a liquid medium, wherein the contents of the vessel are mixed by one or more mechanical stirring elements (e.g., impellars) to maintain uniform distribution of cells, nutrients, gases, and other components. A stirred tank bioreactor is operated under controlled conditions including temperature and pH.

As used herein, the phrase “subject in need” of treatment for a particular condition is a subject having that condition, diagnosed as having that condition, or at risk of developing that condition. According to some embodiments, the phrase “subject in need” of such treatment also is used to refer to a patient who (i) will be administered a composition of the described invention; (ii) is receiving a composition of the described invention; or (iii) has received at least one a composition of the described invention, unless the context and usage of the phrase indicates otherwise.

The term “superoxide dismutases” or “SODs” as used herein refers to major antioxidant defense systems against superoxide anions, which consist of three isoforms of SOD in mammals: the cytoplasmic Cu/ZnSOD (SOD1), the mitochondrial MnSOD (SOD2), and the extracellular Cu/ZnSOD (SOD3), all of which require catalytic metal (Cu or Mn) for their activation. Evidence suggests that in each subcellular location, SODs catalyze the conversion of O2·- H2O2, which may participate in cell signaling. In addition, SODs play a critical role in inhibiting oxidative inactivation of nitric oxide, thereby preventing peroxynitrite formation and endothelial and mitochondrial dysfunction.

Superoxide dismutase (SOD) is widely distributed in plants, animals, and microorganisms [Zheng, M. Antioxidants (Basel) (2023) 12 (9): 1675, citing Zelko, IN., et al. Free Radic. Biol. Med. (2002) 33: 337-49]. Intracellular and extracellular O2·- is produced by plasma membrane-bound NADPH oxidase (NOX). Extracellular H₂O₂ is transported to cells through the aquaporin (AQP) channel and converted to water by the activity of catalase (CAT), peroxidase (PRX) and glutathione peroxidase (GPX) [Id., citing Wu, C. et al. Nano Today (2022) 46: 101574]. When human skin is in direct contact with oxygen, it will cause skin aging and damage [Id., citing Altobelli, G G., et al. Front. Med. (2020) 7: 183]. SOD is a powerful oxygen-free radical scavenger, which has sunscreen and radiation protection effects.

As used herein, the term “super-resolution nanoimaging” refers to an advanced microscopy technique that surpasses the traditional light diffraction limit (~200-300 nm), thereby achieving imaging resolutions beyond the traditional limits of optical microscopy and allowing imaging of biological structures at the nanometer scale. Fluorescent super-resolution nanoimaging manipulates fluorescent nanoparticle fluorophores (e.g., photoswitching or photoblinking for spatial resolution to localize single molecules) or uses specialized light excitation patterns, to provide high-resolution, molecule specific details. [See Li, W. et al. Chem. Rev. (2022) 122 (15): 12495-12543]

The term “suspension” as used herein refers to a dispersion (mixture) in which a finely divided species is combined with another species, with the former being so finely divided and mixed that it doesn't rapidly settle out. In everyday life, the most common suspensions are those of solids in liquid.

The term “symptom” as used herein refers to a sign or an indication of disorder or disease, especially when experienced by an individual as a change from normal function, sensation, or appearance.

The term “synthetic” as used herein refers to production of a substance artificially, e.g., by chemical synthesis, rather than by natural origin.

As used herein, the term “tangential flow filtration” refers to a filtration technique used to separate, purify and concentrate biomolecules by allowing the feed solution to flow parallel to the membrane surface, rather than directly through it. For example, EVs are isolated by passing fluid parallel to a membrane, allowing small molecules to pass through (permeate) while retaining EVs (retentate) based on size. Biological samples are centrifuged at low speeds to remove contaminants, a membrane with a suitable molecular weight cut off or pore size is selected to retain the EVs, while allowing other molecules to pass through, the sample is pumped parallel to the membrane surface and as the fluid flows continuously, molecules smaller than the pores pass into the permeate, while EVs are retained and concentrated in the recirculating retentate.

As used herein, the term “TCF4” refers to a protein that, in conjunction with β-catenin, form a complex within the nucleus resulting in transcriptional activation of downstream genes of the Wnt/β-catenin signaling pathway, a pathway significant in hair physiology. TCF4 is known to be a positive regulator in the maintenance of biological features of DPCs as well as upregulated in DPCs. In addition to its purpose in the biological functions of DPCs, evidence supports a close relation to DPC proliferation as well. When TCF4 is downregulated, hair follicles are phenotypically normal but exist in a prolonged telogen state.

The term “telogen effluvium” refers to a condition resulting from an abrupt shift of large numbers of anagen hairs to telogen hairs on the scalp, with a corresponding change in the ratio of anagen hair to telogen hair from the normal ratio of 90:10 to 70:30. This form of alopecia generally begins approximately 3 months after a major illness or other stress (e.g., surgery, parturition, rapid weight loss, nutritional deficiency, high fever, or hemorrhage) or hormonal derangement; it also has been reported after the initiation of treatment with certain medications.

As used herein, the term “TGFB2” refers to a proteins wherein its synthesis is prompted by dihydrotestosterone (DHT) in DPC at initiation. TGFB2 is suspected to suppress hair elongation; it induces premature catagen of hair follicles in adult hair cycling. It is produced mainly in the dermal papilla region and stimulates the proliferation of hair follicle stem cells (HFSCs). TGFB2 mRNA and protein expression is downregulated in senescent (aging) hDPCs. TGFB2 is involved in the aggregative ability and anti-aging properties of hDPCs through the regulation of collagen genes COL13A1 and COL15A1 expression. Upregulation of these collagen genes increases the spheroidal formation of DPCs and overall hair inductivity. TGFB2 is downregulated in senescent DPCs (aging cells).

The term “TH1 cells” as used herein refers to a lineage of CD4+ effector T cells that promotes cell-mediated immune responses and is required for host defense against intracellular viral and bacterial pathogens. They are mainly involved in activating macrophages but can also help stimulate B cells to produce antibody. TH1 cells secrete IFN-gamma, IL-2, IL-10, and TNF-alpha/beta. IL-12 and IFN-γ make naive CD4+ T cells highly express T-bet and STAT4 and differentiate to TH1 cells [Zhang, Y., et al. Adv. Exp. Med. Bio. (2014) 841: 15-44].

The term “TH2 cells” as used herein refers to a lineage of CD4+ effector T cells that secrete IL-4, IL-5, IL-9, IL-13, and IL-17E/IL-25. These cells are required for humoral or antibody-mediated immunity and play an important role in coordinating the immune response to large extracellular pathogens. IL-4 makes naive CD4+ T cells highly express STAT6 and GATA3 and differentiate to TH2 cells [Zhang, Y. et al. Adv. Exp. Med. Bio. (2014) 841: 15-44].

The term “T_{H}17 cells” as used herein refers to a CD4+ T-cell subset characterized by production of interleukin-17 (IL-17). IL-17 is a highly inflammatory cytokine with robust effects on stromal cells in many tissues, resulting in production of inflammatory cytokines and recruitment of leukocytes, especially neutrophils, thus creating a link between innate and adaptive immunity [Tesmer, LA., et al., Immunol. Rev. (2008) 223: 87-113]. The key transcription factor in T_{H}17 cell development is RORγt.

The term “Treg” or “regulatory T cells” as used herein refers to effector CD4 T cells that inhibit T cell responses and are involved in controlling immune reactions and preventing autoimmunity. The natural regulatory T cell lineage that is produced in the thymus is one subset. The induced regulatory T cells that differentiate from naïve CD4 T cells in the periphery in certain cytokine environments is another subset. Tregs are most commonly identified as CD3+CD4+CD25+FoxP3+ cells in both mice and humans. Additional cell surface markers include CD39, 5′ Nucleotidase/CD73, CTLA-4, GITR, LAG-3, LRRC32, and Neuropilin-1. Tregs can also be identified based on the secretion of immunosuppressive cytokines including TGF-beta, IL-10, and IL-35. Cell surface molecules CTLA-4, LAG-3, and neuropilin-1 (Nrp1) impair dendritic cell (DC)-mediated Tconv activation: CTLA-4 and LAG-3 outcompete CD28 and T cell receptor expressed on conventional T cells for binding to CD80/86 and MHC class II on DCs, and Nrp1 stabilizes DC-Treg contact, thereby preventing antigen presentation to conventional T cells [Ikebuchi, R. et al. Front. Immunol. (2019) doi.org/10.3389/fimmu.2019.01098].

The term “tissue-resident memory T cell” or “T_{RM}” as used herein refers to memory lymphocytes that do not migrate after taking up residence in barrier tissues, where they are retained long term. They appear to be specialized for rapid effector function after restimulation with antigen or cytokines at sites of pathogen entry.

As used herein, the term “therapeutic agent” or “active agent” refers to refers to the ingredient, component or constituent of the compositions of the described invention responsible for the intended therapeutic effect.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest, reduction, or elimination of the progression of a disease manifestation.

As used herein, the term “tissue” refers to a collection of similar cells and the intercellular substances surrounding them. For example, adipose tissue is a connective tissue consisting chiefly of fat cells surrounded by reticular fibers and arranged in lobular groups or along the course of smaller blood vessels. Connective tissue is the supporting or framework tissue of the body formed of fibrous and ground substance with numerous cells of various kinds. It is derived from the mesenchyme, and this in turn from the mesoderm. The varieties of connective tissue include, without limitation, areolar or loose; adipose; sense, regular or irregular, white fibrous; elastic; mucous; lymphoid tissue; cartilage and bone.

The term “tolerance” as used herein refers to the failure to respond to a particular antigen. Tolerance mechanisms that operate in the thymus before the maturation and circulation of T cells are referred to as “central tolerance.” Not all antigens of which T cells need to be tolerant are expressed in the thymus, and therefore central tolerance mechanisms alone are insufficient. Additional tolerance mechanisms exist to restrain the numbers and or function of T cells that are reactive to developmental or food antigens, which are not thymically expressed. Tolerance acquired by mature circulating T cells in the peripheral tissues is called “peripheral tolerance.”

The term “toll-like receptor (TLR)” as used herein refers to innate receptors on macrophages, dendritic cells, and some other cells, that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

The term “topical” as used herein refers to administration of an inventive composition at, or immediately beneath, the point of application. The term “topical administration” and “topically applying” as used herein are used interchangeably to refer to delivering a composition comprising tuned plant exosomes or tuned adipose-derived stem cell (ASC)-derived exosomes comprising a cargo comprising a peptide, a nucleic acid, or both, onto one or more surfaces of a tissue or cell, including epithelial surfaces. The composition may be applied by pouring, dropping, or spraying, if a liquid; rubbing on, if an ointment, lotion, cream, gel, or the like; dusting, if a powder; spraying, if a liquid or aerosol composition; or by any other appropriate means. Topical administration generally provides a local rather than a systemic effect.

Substances generally are applied to the skin to elicit one or more of four general effects: an effect on the skin surface, an effect within the stratum corneum; an effect requiring penetration into the epidermis and dermis; or a systemic effect resulting from delivery of sufficient amounts of a given substance through the epidermis and the dermis to the vasculature to produce therapeutic systemic concentrations. One example of an effect on the skin surface is the formation of a film. Film formation may be protective (e.g., sunscreen) and/or occlusive (e.g., to provide a moisturizing effect by diminishing loss of moisture from the skin surface). One example of an effect within the stratum corneum is skin moisturization; which may involve the hydration of dry outer cells by surface films or the intercalation of water in the lipid-rich intercellular laminae; the stratum corneum also may serve as a reservoir phase or depot wherein topically applied substances accumulate due to partitioning into or binding with skin components.

It generally is recognized that short-term penetration occurs through the hair follicles and the sebaceous apparatus of the skin, while long term penetration occurs across cells. Penetration of a substance into the viable epidermis and dermis may be difficult to achieve, but once it has occurred, the continued diffusion of the substance into the dermis is likely to result in its transfer into the microcirculation of the dermis and then into the general circulation. It is possible, however, to formulate delivery systems that provide substantial localized delivery.

The term “transcellular route” refers to drug permeation through the cells while the term “paracellular route” refers to drug permeation between adjacent cells. Both routes belong to a passive transport process driven by a local concentration gradient. Whether a drug molecule can cross the epithelium and which route it takes are dependent on its intrinsic physicochemical properties. Hydrophilicity/lipophilicity, molecular weight and degree of ionization are the three major determinants. Lipophilic molecules can diffuse freely through the phospholipid bilayers of the cell membrane and therefore prefer the transcellular route. On the other hand, hydrophilic molecules cannot diffuse across the cell membrane, hence the paracellular route becomes significant to these molecules. However, the tight junction between adjacent cells limits the efficacy of this route of transportation. The smaller the molecules, the more effectively they can permeate through the tight junction. The degree of ionization is dependent on the pKa of the drug and the pH of the environment. Only the non-ionized species of a drug can permeate (by drug partitioning) through the cell membrane effectively, and this affects mainly the transcellular route. In general, small lipophilic molecules that are non-ionized at their surrounding pH are favorable for transmucosal absorption. Apart from the three aforementioned properties, aqueous solubility and dose of drug can also influence its absorption. However, absorption also relies on the volume of fluid available at the site of administration for drug dissolution to take place, hence the exact properties of a drug required in order to achieve effective transmucosal delivery is specific to each route of administration.

The term “transcription” as used herein refers to the synthesis of an RNA strand in a process in which ribonucleoside 5′-triphosphates (rNTPs) base-pair sequentially with nucleotides in a template strand and are polymerized in the 5′ to 3′ direction (with elimination of PPi) by an RNA polymerase.

The terms “transdermal delivery system”, transdermal patch” or “patch” refer to an adhesive system placed on the skin to deliver a time released dose of a drug(s) by passage from the dosage form through the skin to be available for distribution via the systemic circulation.

As used herein, the term “transfer RNA” or “tRNA” refers to small, non-coding RNA molecules that act as adaptors during protein synthesis, translating genetic information from mRNA into amino acid sequences

The term “transformation” as used herein refers to the transfer of a nucleic acid molecule into a host organism or host cell by any method. A nucleic acid molecule that has been transformed into an organism/cell may be one that replicates autonomously in the organism/cell, or that integrates into the genome of the organism/cell, or that exists transiently in the cell without replicating or integrating. Non-limiting examples of nucleic acid molecules suitable for transformation are disclosed herein, such as plasmids and linear DNA molecules.

As used herein, the term “transmission electron microscopy” or “TEM” refers to a high resolution imaging technique where a high-energy beam of electrons passes through an ultra-thin specimen to produce an image.

The terms “treat,” “treated,” or “treating” as used herein refers to both therapeutic treatment and/or prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “tune” and its various grammatical forms as used herein, refers to an ability to vary the amount, contents or both of a cargo associated with a population of exosomes for delivery.

As used herein, the term “ultracentrifugation” refers to a high speed separation technique that centrifuges samples at extremely high velocities (i.e., up to 1,000,000×g) to isolate particles based on size, shape, and/or density.

As used herein, the term “ultrafiltration” refers to a membrane filtration process in which concentration gradients lead to a separation through a semipermeable membrane.

The term “underexpressed” as used herein refers to decreased quantity of a gene or gene product relative to the quantity of a gene or gene product under normal conditions.

As used herein, a “unit dose” refers to the amount of inventive composition required to produce a response of 50% of maximal effect (i.e. ED50). The unit dose can be assessed by extrapolating from dose-response curves derived from in vitro or animal model test systems.

The term “variant” as used herein refers to an alteration in the most common DNA nucleoside sequence. The alteration may be benign, pathogenic or of unknown significance.

As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference genome for the species. As used herein, the term “variant allele fraction,” “VAF,” “allelic fraction,” or “AF” refers to the number of times a variant or mutant allele was observed (e.g., a number of reads supporting a candidate variant allele) divided by the total number of times the position was sequenced (e.g., a total number of reads covering a candidate locus).

The term “vascular endothelial growth factor” or “VEGF” as used herein refers to the principal angiogenic growth factor that modulates angiogenesis through receptor tyrosine kinase VEGF receptors (VEGFRs). Multiple VEGFs (e.g., VEGF-A, VEGF-B, VEGF-C, and VEGF-D) interact with VEGFRs, such as VEGFR1, VEGFR2, and VEGFR3. VEGF signaling is induced by the binding of VEGF ligands to their cognate membrane-bound receptors, which results in the activation of multiple downstream pathways. The VEGF signaling cascade includes: the Ras/MAPK pathway, regulating cell proliferation and gene expression; the FAK/paxillin pathway, involved in the rearrangement of the cytoskeleton; the PI3K/AKT pathway, regulating cell survival; and the phospholipase C gamma (PLCγ) pathway, controlling vascular permeability

As used herein, the term “vascular plant” refers to plants characterized by the presence of conducting tissue, which are eukaryotes. They include all the seed-containing plants, angiosperms (flowering plants), gymnosperms, and the pteridophytes (lycophytes, horsetails, and ferns). The vascular tissue of vascular plants includes xylem and phloem. Xylem is vascular tissue that transports water and dissolved minerals from roots to stems and leaves. This type of tissue consists of dead cells that lack end walls between adjacent cells. The side walls are thick and reinforced with lignin, which makes them stiff and waterproof Phloem is vascular tissue that transports food (sugar dissolved in water) from photosynthetic cells to other parts of the plant for growth or storage. This type of tissue consists of living cells that are separated by end walls with tiny perforations, or holes. The vascular tissue allows plants to grow tall in the air without drying out and to take advantage of sunlight. Vascular plants reproduce through a process known as alternation of generations, which involves a life cycle from the haploid phase to the diploid phase and vice versa. While studying the vascular plant life cycle, the diploid phase is considered as the sporophyte and the haploid phase as the gametophyte. Sporophytes are plants that produce spores through meiosis to produce gametophytes further. The period of each phase during the life cycle depends on the type of vascular plant.

The three organs of vascular plants are roots, stems and leaves. Leaves acquire sunlight and carry out photosynthesis to fees the organism; roots explore the soil and acquire the waters and nutrients required for photosynthesis and growth; stem connect the photosynthetic part with the water and nutrient acquiring part and also serve to distribute the leaves effectively in their aerial environment. Each of these three organs possess three fundamental tissues: a ‘skin’ (dermal tissue), transport tissue (vascular tissue), and ground tissue (everything else, i.e., the tissue that fills the spaces between dermal tissue and vascular tissue). Cells of vascular plants show substantially more specialization than is found in non-vascular plants and multiple cell types have been defined, primarily on the basis of:
(1) whether the plant cell is alive or dead at maturity;
(2) cell wall characteristics: all plant cells have what is called a primary cell wall composed of cellulose microfibrils embedded in a matrix of hemicellulose and pectins, molecules that bind cellulose microfibrils to each other and also absorb water, forming a gel. The primary cell wall is present as the cell is growing; when the cell expands the wall yields to the pressures that are present inside the cell. The cell stops growing when the cell wall stiffens and no longer yields to the pressures generated inside it. At this point some cells deposit a distinct type of cell wall material, called a secondary cell wall, inside the primary cell wall. Since the cell is not growing, the more secondary cell wall that is deposited, the smaller the space inside the cell wall becomes. When the cell dies, this space, where the cytosol (usually with a large vacuole) used to be, is termed the lumen. Like the primary cell wall, the secondary cell wall contains cellulose microfibrils, but they are embedded in a matrix of lignin. Lignin is a complex polymer composed of phenolic subunits. Unlike the primary cell wall, the secondary cell wall has substantial compressive strength and does not need a cell membrane and the pressurization of water inside the cell in order for the cell to resist compression. Plants or plant parts (e.g., spinach and many other leaves) with cells possessing only a primary cell wall are called herbaceous and are much less resistant to forces produced by gravity or the wind. Such plants/plant parts lose all structural integrity if the cell membrane is destroyed or if lost water is not replaced; and
(3) cell shape: plant cells come in a variety of shapes. Many cells are round or nearly so, or rectangular with their long dimension being two to ten times that of the short dimensions. Other cells are very elongate with their long dimension being up to 1000 times that of their diameter. Generally, the long axis of cells runs the same direction as the long axis of the plant, i.e., up and down the stem/root.

The term “vitamin” as used herein, refers to any of various organic substances essential in minute quantities to the nutrition of most animals. Vitamins act especially as coenzymes and precursors of coenzymes in the regulation of metabolic processes. Non-limiting examples of vitamins usable in context of the described invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B3 (niacinamide and its derivatives), alpha hydroxy acids (such as glycolic acid, lactic acid, tartaric acid, malic acid, citric acid, etc.) and beta hydroxy acids (such as salicylic acid and the like).

The term “vesicle flow cytometry”, as used herein refers to a high-throughput, single-particle technique used to characterize and quantify extracellular vesicles (EVs), such as exosomes, typically ranging from 30 to 1000 nm. It enables rapid, simultaneous analysis of surface markers and internal cargo, crucial for identifying cancer or disease biomarkers. Specialized high-sensitivity cytometers are needed to overcome the challenges posed by the small size and low refractive index of vesicles. For example, the Beckman Coulter CytoFLEX instrument enables detection of EVs to ~75 nm and detection of EV surface antibodies to ~25 molecules per vesicle (beckman.it/en/resources/reading-material/application-notes/vesicle-flow-cytometry).

The term “western blot” refers to a laboratory technique used to detect specific proteins. In a western blot, proteins in a mixture are separated by size using gel electrophoresis, the proteins are then transferred onto a membrane, wherein they are incubated with antibodies, and visualized using e.g., chemiluminescence or fluorescence to confirm protein presence and quantity.

The term “wound healing” refers to the process by which the body repairs trauma to any of its tissues, especially those caused by physical means and with interruption of continuity. A wound-healing response often is described as having three distinct phases-injury, inflammation and repair. Generally speaking, the body responds to injury with an inflammatory response, which is crucial to maintaining the health and integrity of an organism. If, however, it goes awry, it can result in tissue destruction. Although these three phases are often presented sequentially, during chronic or repeated injury, these processes function in parallel, placing significant demands on regulatory mechanisms. (Wilson and Wynn, Mucosal Immunol., 2009, 3(2): 103-121).

### Phase I: Injury

Injury caused by factors including, but not limited to, autoimmune or allergic reactions, environmental particulates, or infection or mechanical damage, often results in the disruption of normal tissue architecture, initiating a healing response. Damaged epithelial and endothelial cells must be replaced to maintain barrier function and integrity and prevent blood loss, respectively. Acute damage to endothelial cells leads to the release of inflammatory mediators and initiation of an anti-fibrinolytic coagulation cascade, temporarily plugging the damaged vessel with a platelet and fibrin-rich clot. For example, lung homogenates, epithelial cells or bronchoalveolar lavage fluid from idiopathic pulmonary fibrosis (IPF) patients contain greater levels of the platelet-differentiating factor, X-box-binding protein-1, compared with chronic obstructive pulmonary disease (COPD) and control patients, suggesting that clot-forming responses are continuously activated. In addition, thrombin (a serine protease required to convert fibrinogen into fibrin) is also readily detected within the lung and intra-alveolar spaces of several pulmonary fibrotic conditions, further confirming the activation of the clotting pathway. Thrombin also can directly activate fibroblasts, increasing proliferation and promoting fibroblast differentiation into collagen-producing myofibroblasts. Damage to the airway epithelium, specifically alveolar pneumocytes, can evoke a similar anti-fibrinolytic cascade and lead to interstitial edema, areas of acute inflammation, and separation of the epithelium from the basement membrane.

Platelet recruitment, degranulation and clot formation rapidly progress into a phase of vasoconstriction with increased permeability, allowing the extravasation (movement of white blood cells from the capillaries to the tissues surrounding them) and direct recruitment of leukocytes to the injured site. The basement membrane, which forms the extracellular matrix underlying the epithelium and endothelium of parenchymal tissue, precludes direct access to the damaged tissue. To disrupt this physical barrier, zinc-dependent endopeptidases, also called matrix metalloproteinases (MMPs), cleave one or more extracellular matrix constituents allowing extravasation of cells into, and out of, damaged sites.

### Phase II: Inflammation

Once access to the site of tissue damage has been achieved, chemokine gradients recruit inflammatory cells. Neutrophils, eosinophils, lymphocytes, and macrophages are observed at sites of acute injury with cell debris and areas of necrosis cleared by phagocytes.

The early recruitment of eosinophils, neutrophils, lymphocytes, and macrophages providing inflammatory cytokines and chemokines can contribute to local TGF-β and IL-13 accumulation. Following the initial insult and wave of inflammatory cells, a late-stage recruitment of inflammatory cells may assist in phagocytosis, in clearing cell debris, and in controlling excessive cellular proliferation, which together may contribute to normal healing. Late-stage inflammation may serve an anti-fibrotic role and may be required for successful resolution of wound-healing responses. For example, a late-phase inflammatory profile rich in phagocytic macrophages, assisting in fibroblast clearance, in addition to IL-10-secreting regulatory T cells, suppressing local chemokine production and TGF-β, may prevent excessive fibroblast activation.

The nature of the insult or causative agent often dictates the character of the ensuing inflammatory response. For example, exogenous stimuli like pathogen-associated molecular patterns (PAMPs) are recognized by pathogen recognition receptors, such as toll-like receptors and NOD-like receptors (cytoplasmic proteins that have a variety of functions in regulation of inflammatory and apoptotic responses), and influence the response of innate cells to invading pathogens. Endogenous danger signals also can influence local innate cells and orchestrate the inflammatory cascade.

The nature of the inflammatory response dramatically influences resident tissue cells and the ensuing inflammatory cells. Inflammatory cells themselves also propagate further inflammation through the secretion of chemokines, cytokines, and growth factors. Many cytokines are involved throughout a wound-healing and fibrotic response, with specific groups of genes activated in various conditions. Fibrotic lung disease (such as idiopathic pulmonary fibrosis) patients more frequently present pro-inflammatory cytokine profiles (including, but not limited to, interleukin-1 alpha (IL-1α), interleukin-1 beta (IL-1β), interleukin-6 (IL-6), tumor necrosis factor alpha (TNF-α), transforming growth factor beta (TGF-β), and platelet-derived growth factors (PDGFs)). Each of these cytokines has been shown to exhibit significant pro-fibrotic activity, acting through the recruitment, activation and proliferation of fibroblasts, macrophages, and myofibroblasts.

### Phase I: Tissue Repair and Contraction

The closing phase of wound healing consists of an orchestrated cellular reorganization guided by a fibrin (a fibrous protein that is polymerized to form a “mesh” that forms a clot over a wound site)-rich scaffold formation, wound contraction, closure and re-epithelialization. The vast majority of studies elucidating the processes involved in this phase of wound repair have come from dermal wound studies and in vitro systems.

Myofibroblast-derived collagens and smooth muscle actin (α-SMA) form the provisional extracellular matrix, with macrophage, platelet, and fibroblast-derived fibronectin forming a fibrin scaffold. Collectively, these structures are commonly referred to as granulation tissues. Primary fibroblasts or alveolar macrophages isolated from IPF patients produce significantly more fibronectin and α-SMA than control fibroblasts, indicative of a state of heightened fibroblast activation. It has been reported that IPF patients undergoing steroid treatment had similar elevated levels of macrophage-derived fibronectin as IPF patients without treatment. Thus, similar to steroid resistant IL-13-mediated myofibroblast differentiation, macrophage-derived fibronectin release also appears to be resistant to steroid treatment, providing another reason steroid treatment may be ineffective. From animal models, fibronectin appears to be required for the development of pulmonary fibrosis, as mice with a specific deletion of an extra type III domain of fibronectin (EDA) developed significantly less fibrosis following bleomycin administration compared with their wild-type counterparts.

In addition to fibronectin, the provisional extracellular matrix consists of glycoproteins (such as PDGF), glycosaminoglycans (such as hyaluronic acid), proteoglycans and elastin. Growth factor and TGF-β-activated fibroblasts migrate along the extracellular matrix network and repair the wound. Within skin wounds, TGF-β also induces a contractile response, regulating the orientation of collagen fibers. Fibroblast to myofibroblast differentiation, as discussed above, also creates stress fibers and the neo-expression of α-SMA, both of which confer the high contractile activity within myofibroblasts. The attachment of myofibroblasts to the extracellular matrix at specialized sites called the “fibronexus” or “super mature focal adhesions” pull the wound together, reducing the size of the lesion during the contraction phase. The extent of extracellular matrix laid down and the quantity of activated myofibroblasts determines the amount of collagen deposition. To this end, the balance of matrix metalloproteinases (MMPs) to tissue inhibitor of metalloproteinases (TIMPs) and collagens to collagenases vary throughout the response, shifting from pro-synthesis and increased collagen deposition towards a controlled balance, with no net increase in collagen. For successful wound healing, this balance often occurs when fibroblasts undergo apoptosis, inflammation begins to subside, and granulation tissue recedes, leaving a collagen-rich lesion. The removal of inflammatory cells, and especially α-SMA-positive myofibroblasts, is essential to terminate collagen deposition.

The term “zwitterionic”, as used herein, refers or pertains to a molecule that carries both positive and negative charged functional groups, resulting in an overall neutral charge.

### EMBODIMENTS

### Compositions of the Present Disclosure

The present disclosure provides a composition comprising a purified population of extracellular vesicles (EVs) isolated from one or more biological samples, wherein the size of the EVs is from about 50 nm to about 200 nm, inclusive, wherein the purified population of EVs comprise a tuned cargo comprising:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p; and
(ii) a signature of one or more proteins selected from the group consisting of BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, WNT5A, wherein the tuned cargo of the EVs is produced by exposure of the one or more biological samples to one or more stress conditions that cause the biological sample to modulate the EV cargo composition.

### Sources of the Biological Sample

The present disclosure provides compositions comprising a population of extracellular vesicles (EVs). In some embodiments, the population of EVs is derived from a biological sample.

In some embodiments, the biological sample is a population of cells maintained in culture. In some embodiments, the population of cells maintained in culture is a heterogenous population of cells. In some embodiments, the population of cells maintained in culture is a homogenous population of cells. In some embodiments, the population of cells maintained in culture is derived from an immortalized cell line (i.e., cells that have been modified to proliferate indefinitely). In some embodiments, the population of cells maintained in culture is derived from a primary cell line (i.e., cells that are isolated directly from living tissue).

In some embodiments, the population of cells maintained in culture comprises stem and progenitor cells (i.e., unspecialized cells capable of self-renewing (divide indefinitely) and differentiating into various cell types). Nonlimiting examples of stem and progenitor cells include mesenchymal stem cells (i.e., multipotent, self-renewing adult stem cells found in bone marrow, adipose tissue, and umbilical cord blood), adipose-derived stem cells (i.e., adult, multipotent stem cells (mesenchymal stem cells) found in high concentrations within human fat tissue), hematopoietic stem cells (i.e., immature blood-forming cells found in bone marrow, peripheral blood, and umbilical cord blood), neural stem cells (i.e., specialized, multipotent cells in the nervous system that possess the unique ability to self-renew and differentiate into the three primary cell types of the brain), epithelial stem cells (i.e., tissue-specific stem cells responsible for the long-term self-renewal, maintenance, and repair of epithelial tissues, such as the skin, intestines, and cornea), endothelial stem cells (i.e., multipotent adult stem cells—primarily located in the bone marrow—that self-renew and differentiate into endothelial cells (ECs), which form the inner lining of blood and lymphatic vessels), endothelial progenitor cells (i.e., rare, immature stem cells circulating in the blood that repair damaged blood vessels and form new ones), dermal stem cells (i.e., multipotent adult stem cells located in the dermis (inner layer) of the skin, particularly within hair follicles (dermal papilla/sheath)), dermal progenitor cells (i.e., specialized, multipotent adult stem cells located within the skin's dermal layer), hair follicle stem cells (i.e., multipotent adult stem cells residing in the follicle's bulge region, essential for continuous hair regeneration and skin repair), and myosatellite cells (i.e., small, multipotent stem cells found in mature skeletal muscle, specifically located between the basement membrane and sarcolemma of muscle fibers).

In some embodiments, the population of cells maintained in culture comprises immune cells (i.e., specialized cells derived from stem cells in the bone marrow that defend the body against infections, foreign invaders, and diseases). Nonlimiting examples of immune cells include peripheral blood mononuclear cells (i.e., specialized white blood cells with a single, round nucleus, critical to the human immune system), T cells (i.e., specialized white blood cells and a critical component of the adaptive immune system, produced in the bone marrow and matured in the thymus), B cells (i.e., a crucial type of white blood cell that act as a primary component of the adaptive immune system. Their main function is to protect the body from infections by producing antibodies), NK cells (i.e., a type of white blood cell and a crucial part of the innate immune system, acting as a rapid, first-line defense against viruses and cancerous cells), monocytes (i.e., a type of large white blood cell (leukocyte) that act as a crucial part of the innate immune system, fighting infections and removing dead or damaged tissues), macrophages (i.e., a large phagocytic cell found in stationary form in the tissues or as a mobile white blood cell, especially at sites of infection), dendritic cells (i.e., specialized, bone marrow-derived immune cells (leukocytes) that act as the primary “sentinels” of the immune system by capturing pathogens and presenting antigens to T cells to initiate adaptive immunity), neutrophils (i.e., the most abundant type of white blood cell, acting as the immune system's first line of defense against infections), eosinophils (i.e., a type of disease-fighting white blood cell (granulocyte) that form part of the immune system), basophils (i.e., a type of white blood cell (leukocyte) that function as part of the innate immune system to defend against parasites, bacteria, and allergens), and mast cells (i.e., essential immune system cells, derived from bone marrow and residing in connective tissues throughout the body).

In some embodiments, the population of cells maintained in culture comprises blood and circulating cells (i.e., a specialized, circulating fluid tissue and cells that transports oxygen, nutrients, and wastes throughout the body). Nonlimiting examples of blood and circulating cells include erythrocytes (i.e., specialized, disk-shaped cells produced in the bone marrow that transport oxygen from the lungs to body tissues and bring carbon dioxide back for exhalation), leukocytes (i.e., essential immune system cells produced in bone marrow that circulate throughout the bloodstream and tissues to defend the body against infections, bacteria, viruses, and foreign invaders), thrombocytes (i.e., tiny, disc-shaped cell fragments in the blood produced in bone marrow that are essential for clotting and wound healing), and circulating tumor cells (i.e., cancer cells that have detached from a primary solid tumor, entered the bloodstream, and circulate throughout the body).

In some embodiments, the population of cells maintained in culture comprises stromal and connective tissue cells (i.e., cells that form the structural framework of organs, providing essential support, nourishment, and regulatory signals). Nonlimiting examples of stromal and connective tissue cells include fibroblasts (i.e., essential for maintaining structural integrity by producing extracellular matrix (ECM) components like collagen and elastin), adipocytes (i.e., specialized cells that primarily compose adipose tissue, functioning as the body's main energy reservoir by storing fat as triglycerides), pericytes (i.e., multifunctional, perivascular cells embedded within the basement membrane of capillaries and venules), chondrocytes (i.e., the primary cells found in healthy cartilage, responsible for producing and maintaining the extracellular matrix (ECM)), osteoblasts (i.e., a cell that secretes the matrix for bone formation), and osteoclasts (i.e., a large multinucleate bone cell which absorbs bone tissue during growth and healing).

In some embodiments, the population of cells maintained in culture comprises epithelial cells (i.e., a diverse group of tightly packed cells forming continuous, protective layers that cover internal and external body surfaces, including skin, organs, and glands). Nonlimiting examples of epithelial cells include squamous cells (i.e., thin, flat cells that look like fish scales under a microscope, forming the outer layer of the skin (epidermis) and lining hollow organs, respiratory passages, and digestive tracts), cuboidal cells (i.e., box-shaped, cube-like epithelial cells with roughly equal width, height, and depth, featuring a large, central, spherical nucleus), columnar cells (i.e., tall, narrow epithelial cells that resemble columns, with elongated nuclei usually located near the base), and transitional cells (i.e., specialized, flexible epithelial cells that line the urinary tract, including the renal pelvis, ureters, bladder, and part of the urethra).

In some embodiments, the population of cells maintained in culture comprises endothelial and vascular cells (i.e., form a thin, specialized, flat monolayer (the endothelium) that acts as the inner lining of all blood and lymphatic vessels, placing them in direct contact with blood and lymph). Nonlimiting examples of endothelial and vascular cells include vascular endothelial cells (i.e., a thin, specialized layer of cells (the endothelium) lining the entire circulatory system, including the heart, blood vessels, and lymphatic vessels), lymphatic endothelial cells (i.e., specialized cells forming the inner lining of lymphatic vessels and lymph node sinuses, crucial for regulating fluid balance, immune cell trafficking, and tissue immune surveillance), and endocardial cell (i.e., specialized, flat, squamous endothelial cells forming the innermost layer (endocardium) of the heart wall, separating blood from the heart muscle).

In some embodiments, the population of cells maintained in culture comprises neural and glial cells (i.e., the two primary cell types in the nervous system. Neurons transmit electrical and chemical signals to process information, while glial cells provide essential support, protection, and nutrition for neurons). Nonlimiting examples of neural and glial cells include neurons (i.e., the fundamental, electrically excitable working units of the brain and nervous system that receive, process, and transmit information throughout the body using electrical and chemical signals), astrocytes (i.e., the most abundant star-shaped glial cells in the central nervous system, providing essential structural, nutritional, and metabolic support to neurons), oligodendrocytes (i.e., specialized glial cells in the central nervous system (CNS) that form the myelin sheath, a fatty, insulating layer around neuronal axons), and microglia (i.e., specialized resident immune cells (macrophages) in the central nervous system (CNS) that act as the brain's primary defense).

In some embodiments, the population of cells maintained in culture comprises muscle cells (i.e., a specialized contractile cell in animal muscle tissue designed for generating force and movement). Nonlimiting examples of muscle cells include skeletal muscle cells (i.e., a long, cylindrical, multinucleated, and striated cell designed for voluntary contraction and force generation), smooth muscle cells (i.e., a type of non-striated, involuntary muscle cell characterized by a fusiform (spindle-shaped) structure, which is thick in the middle and tapered at both ends), and cardiomyocytes (i.e., specialized, involuntary muscle cells forming the heart's muscular layer (myocardium) that enable it to contract and pump blood).

In some embodiments, the population of cells maintained in culture comprises cancer or diseased cells. Nonlimiting examples of cancer or diseased cells include tumor cells (i.e., abnormally dividing, mutated cells that accumulate when old cells fail to die and new, unnecessary cells continue to form), cancer stem cells (i.e., a small subpopulation of cells within tumors that possess unique abilities to self-renew, differentiate into multiple cell types, and drive tumor growth), infectious cells (i.e., a host cell that has been invaded by a pathogen and has been hijacked to produce or transport new infectious agents), and adapted cells (i.e., cells that have undergone structural or functional changes to survive, function, or maintain stability under new or stressful environmental conditions).

In some embodiments, the biological sample is a population of cells maintained as organoids (i.e., 3D, miniaturized, and simplified versions of organs grown in vitro). In some embodiments, the organoids comprise cells derived from a subject. In some embodiments, the organoids comprise cells derived from cultured cells.

In some embodiments, the biological sample is a tissue. In some embodiments, the tissue is human tissue. In some embodiments, the tissue comprises tissue explants maintained ex vivo. In some embodiments, the tissue is derived from a subject. In some embodiments, the subject is a normal healthy subject. In some embodiments, the subject is a subject having or suspected of having a disease. According to some embodiments, the subject is a mammal. In some embodiments, the subject is a non-human mammal or a human subject.

In some embodiments, the tissue comprises epithelial tissue (i.e., tissue that covers exterior surfaces, lines internal cavities, and forms glands). Nonlimiting examples of epithelial tissue include simple squamous epithelium (i.e., a type of tissue composed of thin, flat, scale-like cells that form protective barriers or facilitate diffusion in the body), stratified squamous epithelium (i.e., a multi-layered tissue composed of flattened, scale-like cells at the surface and cuboidal/columnar cells at the base), simple cuboidal epithelium (i.e., a single layer of cube-shaped cells, roughly as wide as they are tall), stratified cuboidal epithelium (i.e., a type composed of two or more layers of cube-shaped cells), simple columnar epithelium (i.e., a single layer of tall, narrow epithelial cells specialized from absorption, secretion of mucus/enzymes, and protection), pseudostratified columnar epithelium (i.e., a specialized, single-layered tissue that appears stratified), and transitional epithelium (i.e., a specialized, multi-layered tissue that lines the urinary tract). In some embodiments, the tissue comprises connective tissue (i.e., tissues that bind, support, protect, and integrate, or separate other tissues). Nonlimiting examples of connective tissue include loose connective tissue (i.e., a flexible, gel-like, vascularized tissue that cushions organs, binds skin, and holds tissue together), dense connective tissue (i.e., a strong, fibrous tissue high in collagen fibers that provides structural support, protection, and flexibility to the body), adipose tissue (i.e., a specialized connective tissue composed mainly of fat-storing cells), bone (i.e., a specialized, mineralized connective tissue that forms the structural framework of the skeleton), cartilage (i.e., a tough, flexible, and resilient connective tissue that cushions joints, reduces friction between bones, and provides structural support), and blood (i.e., a specialized, liquid connective tissue that circulates through the body's vessels). In some embodiments, the tissue comprises muscle tissue (i.e., tissue that is composed of elongated, contractile cells that generate force through contraction, enabling movement). Nonlimiting examples of muscle tissue includes skeletal muscle (i.e., a voluntary, striated muscle type attached to bones by tendons, forming bundles of muscle fibers that control body movement, posture, and heat generation), smooth muscle (i.e., an involuntary, non-striated muscle found in the walls of hollow organs, blood vessels, and airways, designed to manage passage width and transport substances), and cardiac muscle (i.e., a specialized, involuntary, striated muscle tissue found exclusively in the heart walls).

In some embodiments, the biological sample is a plant. In some embodiments, the biological sample is a tissue from a vascular plant. In some embodiments, the tissue from a vascular plant includes, but is not limited to roots, stems, leaves, flowers, seeds, fruits, liquid extracts of any of the foregoing, or a combination thereof.

In some embodiments, the plant belongs to the following plant families: Asphodelaceae, Solanaceae, Curcurbitaceae, Fabaceae, Meliaceae, Oleaceae, Asteraceae, Plumbaginaceae, Juglandaceae, Droseraceae, Polygonaceae, Rosaceae, Liliaceae, Orobanchaceae, Apiaceae, Betulaceae, Passifloraceae, Papaveraeeae, Lamiaceae, Rubiaceae, Alariaceae, Laminariaceae, Lessoniceae, Ulvaceae, Gigartinaceae, Passiflora ligularis (granadilla), Passiflora edulis, Morinda citrifolia (Noni), Agastache Mexicana, Laminaria Digitata, Chondrus crispus, Macrocystis pyrifera, Asphodelacae, Xanthorrhoeaceae, Hemerocallidoideae, Asphodeloideae, Aloe Vera, Mascarene Aloes, Aloe Barbadensis Miller, Aloe Ferox, Aloe Arborescens, Aloe Perryi Baker, Aloe Succotrina Weston, Aloe Maculata All., Cape Aloe, Aloe tormentorri, Aloe purpurea, Aloe macra, Aloe lomatophylloides, Aloe macra Haw, Haworthia, Gasteria, Xanthorrhoea, Vicia Faba Viciafaba L. minor, Lansium Domesticum, Momordica Charantia, Syringa Vulgaris, Artemesia, Artemisia Absinthium, Artemisia Vulgaris, Sagebrush Artemisia species, Plumbago, Juglans nigra, Genus: Drosera, Drosera rotundifolia, Drosera intermedia, Drosera anglica, Drosera ramentacea, Drosera madagascariensis, Lustwort, Reynoutria multiflora, Fallopia multiflora, Polygonum multiflorum, Prunus persica, Pumpkin, Passiflora ligularis, Morinda citrifolia (Noni), Agastache Mexicana, Laminaria Digitata, Chondrus crispus, Macrocystis pyrifera, Alariaceae, Laminariaceae, Lessoniceae, Ulvaceae, Gigartinaceae, Curcuma longa, Solanaceae, Curcurbitaceae, Chelidonium majus, Lilium lancifolium, Lilium tigrinum, Rehmanniae Radix, Centella asiatica, and Betula pendula.

In some embodiments, the compositions, as disclosed herein, comprise a population of EVs derived from a plurality of biological samples. In some embodiments, the plurality of biological samples comprises two or more biological samples. In some embodiments, the plurality of biological samples comprises three or more biological samples. In some embodiments, the plurality of biological samples comprises four or more biological samples. In some embodiments, the plurality of biological samples comprises five or more biological samples. In some embodiments, the plurality of biological samples comprises six or more biological samples. In some embodiments, the plurality of biological samples comprises seven or more biological samples. In some embodiments, the plurality of biological samples comprises eight or more biological samples. In some embodiments, the plurality of biological samples comprises nine or more biological samples. In some embodiments, the plurality of biological samples comprises ten or more biological samples.

In some embodiments, the compositions comprise a population of EVs derived from a plurality of biological samples comprising a tissue. In some embodiments, the compositions comprise a population of EVs derived from a plurality of biological samples comprising a population of cells derived from cells maintained in culture. In some embodiments, the compositions comprise a population of EVs derived from a plurality of biological samples comprising a plant. In some embodiments, the compositions comprise a population of EVs derived from a plurality of biological samples comprising a tissue, a population of cells derived from cells maintained in culture, a plant, or a combination thereof.

In some embodiments, the compositions comprise a population of EVs derived from adipose-derived stem cells (ASCs). In some embodiments, the ASCs are derived from adipose tissue. In some embodiments, the adipose tissue is derived from a subject. In some embodiments, the subject is a human or a non-human mammal. In some embodiments, the adipose tissue is a tissue explant from a subject that is maintained ex vivo. In some embodiments, the compositions comprise a population of EVs derived from human ASCs and Aloe vera plants.

In some embodiments, the ASCs retain mesenchymal stromal cell (MSC) characteristics comprising: (i) adherence to plastic in standard culture conditions; (ii) a surface marker phenotype comprising CD105+, CD73+, CD90+, CD45−, CD34−, CD14− or CD11b−, CD79α− or CD19−, and HLA-DR−; and (iii) a capacity to differentiate into bone, adipose, and cartilage cells.

### Stress Conditioning

In some embodiments, a composition comprises a population of EVs derived from a biological sample, wherein the biological sample is exposed to one or more stress conditions.

In some embodiments, the one or more stress conditions is sufficient to modulate the cargo of EVs derived from the biological sample. In some embodiments, the one or more stress conditions is sufficient to modulate the biological sample's gene expression, signaling pathways, metabolism, cellular responses, or a combination thereof. In some embodiments, exposure to the one or more stress conditions produces a population of EVs comprising a “tuned” cargo.

In some embodiments, the one or more stress conditions comprises hypoxic stress (i.e., a state of low oxygen). In some embodiments, the one or more stress conditions comprises metabolic stress (i.e., a state of nutrient scarcity or high energy demand). In some embodiments, the one or more stress conditions comprises mechanical stress (i.e., when physical forces are applied). In some embodiments, the one or more stress conditions comprises osmotic stress (i.e., sudden changes in solute concentration, disrupting water balance). In some embodiments, the one or more stress conditions comprises chemical stress (i.e., exposure to toxic agents). In some embodiments, the one or more stress conditions comprises heat stress (i.e., exposure to excessive temperatures).

In some embodiments, the composition comprises a population of EVs derived from a biological sample, wherein the biological sample is exposed to hypoxic stress comprising exposure to a reduced oxygen environment relative to ambient atmospheric oxygen levels, wherein the reduced oxygen environment comprises an oxygen concentration of about 0.1% to about 5%, inclusive, i.e., about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0%, inclusive.

In some embodiments, the biological sample is exposed to reduced oxygen conditions for about 1 hour to about 72 hours, inclusive, i.e., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, or 72 hours.

In some embodiments, the composition comprises a population of EVs derived from a biological sample, wherein the biological sample is exposed to metabolic stress comprising exposure to glucose deprivation, amino acid deprivation, lipid deprivation, protein deprivation, or a combination thereof.

In some embodiments, the biological sample is exposed to glucose deprivation, amino acid deprivation, lipid deprivation, protein deprivation, or a combination thereof for about 1 hour to about 72 hours, inclusive, i.e., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, or 72 hours.

In some embodiments, the composition comprises a population of EVs derived from a biological sample, wherein the biological sample is exposed to osmotic stress comprising exposure to hypotonic (i.e., low solute) or hypertonic (i.e., high solute) solutions, forcing rapid water movement across the membrane.

In some embodiments, the biological sample is exposed to hypertonic or hypotonic solutions for a period of time between 10 minutes to about 6 hours, inclusive, i.e., about 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, or 6 hours.

In some embodiments, the composition comprises a population of EVs derived from a biological sample, wherein the biological sample is exposed to mechanical stress comprising exposure to shear stress, stretching, compression, agitation, vibration, pressure changes, or a combination thereof.

In some embodiments, the biological sample is exposed to shear stress, stretching, compression, agitation, vibration, pressure changes, or a combination thereof for about 10 minutes to about 1 month, inclusive, i.e., 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, 6 hours, 7 hours, 8 hours, 9 hours, 10 hours, 11 hours, 12 hours, 14 hours, 16 hours, 18 hours, 20 hours, 22 hours, 1 day, 2 days, 3 days, 4 days, 5 days, 6 days, 7 days, 1 week, 2 weeks, 3 weeks, or 1 month.

In some embodiments, the composition comprises a population of EVs derived from a biological sample, wherein the biological sample is exposed to chemical stress comprising exposure to cytokines, growth factors, reactive oxygen species, pharmacological agents, toxins, pH altering agents, or a combination thereof.

In some embodiments, the biological sample is exposed to cytokines, growth factors, reactive oxygen species, pharmacological agents, toxins, pH altering agents, or a combination thereof for a time period between 1 hour to about 72 hours, inclusive, i.e., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, or 72 hours.

In some embodiments, the composition comprises a population of EVs derived from a biological sample, wherein the biological sample is exposed to heat stress comprising exposure to high temperatures of about 30 °C to about 50 °C, inclusive, i.e., about 30 °C, 31 °C, 32 °C, 33 °C, 34 °C, 35 °C, 36 °C, 37 °C, 38 °C, 39 °C, 40 °C, 41 °C, 42 °C, 43 °C, 44 °C, 45 °C, 46 °C, 47 °C, 48 °C, 49 °C, or 50 °C.

In some embodiments, the biological sample is exposed to high temperatures for a period of time between 10 minutes to about 6 hours, inclusive, i.e., about 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, or 6 hours.

In some embodiments, the biological sample is subsequently exposed to a temperature of about 36 °C-38 °C, inclusive, i.e., about 36 °C, 37 °C, or 38 °C, for about 24 hours-72 hours, inclusive, i.e., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, or 36 hours.

In some embodiments, the biological sample is subsequently exposed to a temperature of about 18 °C-25 °C, inclusive, i.e., about 18 °C, 19 °C, 20 °C, 21 °C, 22 °C, 23 °C, 24 °C, or 25 °C, for about 1 hours-36 hours, inclusive, i.e., about 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, or 72 hours.

In some embodiments, following exposure to the one or more stress conditions, the biological sample is maintained under non-stress conditions for a recovery period. In some embodiments, the recovery period is about 1 hour to about 72 hours, inclusive, i.e., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, or 72 hours.

### Extracellular Vesicle Characteristics

In some embodiments, the composition comprises a population of EVs, wherein the EVs are characterized by purity, size, quantity, surface marker composition, or a combination thereof in order to distinguish the population of EVs from contaminants.

In some embodiments, the population of EVs comprise exosomes. In some embodiments, the population EVs comprise microvesicles. In some embodiments, the population EVs comprise apoptotic bodies.

According to some embodiments, the purity of the population of EVs ranges from at least 50% to 100% pure, i.e., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100%, inclusive. In some embodiments, the population of EVs is substantially free of contaminants, meaning the population includes less than 50%, less than 49%, less than 48%, less than 47%, less than 46%, less than 45%, less than 44%, less than 43%, less than 42%, less than 41%, less than 40%, less than 39%, less than 38%, 3 less than 7%, less than 36%, less than 35%, less than 34%, less than 33%, less than 32%, less than 31%, less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1% by weight of any contaminating forms.

According to some embodiments, compositions comprise a population of EVs, wherein the size of the EVs range from about 30 nm to about 550 nm, inclusive, in diameter, i.e., about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 305 nm, about 310 nm, about 315 nm, about 320 nm, about 325 nm, about 330 nm, about 335 nm, about 340 nm, about 345 nm, about 350 nm, about 355 nm, about 360 nm, about 365 nm, about 370 nm, about 375 nm, about 380 nm, about 385 nm, about 390 nm, about 395 nm, about 400 nm, about 405 nm, about 410 nm, about 415 nm, about 420 nm, about 425 nm, about 430 nm, about 435 nm, about 440 nm, about 445 nm, about 450 nm, about 455 nm, about 460 nm, about 465 nm, about 470 nm, about 475 nm, about 480 nm, about 485 nm, about 490 nm, about 495 nm, about 500 nm, about 505 nm, about 510 nm, about 515 nm, about 520 nm, about 525 nm, about 530 nm, about 535 nm, about 540 nm, about 545 nm, or about 550 nm in diameter, inclusive.

In some embodiments, the population of EVs comprises a quantity from about 1×10E5 to about 1×10E6 EVs, inclusive. In some embodiments, the isolated population of EVs comprises from about 1×10E6 to about 1×10E7 EVs, inclusive. In some embodiments, the isolated population of EVs comprises from about 1×10E7 to about 1×10E8 EVs, inclusive. In some embodiments, isolated population of EVs comprises from about 1×10E8 to about 1×10E9 EVs, inclusive. In some embodiments, the isolated population of EVs comprises from about 1×10E9 to about 1×10E10 EVs, inclusive. In some embodiments, the isolated population of EVs comprises from about 1×10E10 to 1×10E11 EVs, inclusive. In some embodiments, the isolated population of EVs comprises from about 1×10E11 to about 1×10E12 EVs, inclusive. In some embodiments, the isolated population of EVs comprises from about 1×10E12 to about 1×10E13 EVs, inclusive. In some embodiments, the isolated population of EVs comprises from about 1×10E13 to about 1×10E14 EVs, inclusive.

In some embodiments, a surface marker of the population of EVs comprises CD9. In some embodiments, a surface marker of the population of EVs comprises CD63. In some embodiments, a surface marker of the population of EVs comprises CD81. In some embodiments, a surface marker of the population of EVs comprises CD82. In some embodiments, a surface marker of the population of EVs comprises CD9, CD63, CD81, CD82, or a combination thereof.

### Extracellular Vesicle Cargo

In some embodiments, exposure of the biological sample to the one or more stress conditions produces EVs comprising a “tuned” cargo. In some embodiments, the tuned cargo of the EVs comprises an miRNA signature. In some embodiments, the miRNA signature comprises miRNAs that are involved in collagen production, skin barrier function, immune regulation, anti-inflammatory activity, cell proliferation, wound healing, angiogenesis, melanogenesis, pigmentation, photoaging, UV damage, acne formation, hair growth, inhibition of hair loss, or a combination thereof.

In some embodiments, the miRNA signature of the EVs comprises miRNAs that are involved in collagen production. In some embodiments, the human miRNAs that are involved in collagen production comprise hsa-miR-143-3p, hsa-miR-29a-3p, hsa-miR-21-5p, hsa-miR-140-3p, hsa-miR-25-3p, hsa-miR-21-3p, or a combination thereof. In some embodiments, the miRNA signature of the EVs includes one or more functionally equivalent plant miRNAs, where the term “functionally equivalent” as used herein refers to having the same safety profile and cosmetic properties for cleansing, beautifying, promoting attractiveness, or altering the appearance as compositions containing only human miRNAs.

In some embodiments, the miRNA signature of the EVs comprises miRNAs that are involved in skin barrier function. In some embodiments, the human miRNAs that are involved in skin barrier function comprise hsa-miR-143, hsa-miR-155-5p, hsa-miR-146a, or a combination thereof. In some embodiments, the miRNA signature of the EVs includes one or more functionally equivalent plant miRNAs.

In some embodiments, the miRNA signature of the EVs comprises miRNAs that are involved in immune regulation, anti-inflammatory activity, or both. In some embodiments, the human miRNAs that are involved in immune regulation and anti-inflammatory activity comprise hsa-miR-143-3p, hsa-miR-21, let-7i-5p, hsa-miR-199a-3p, hsa-miR-26a, hsa-miR-29a-3p, hsa-miR-27b, hsa-miR-155-5p, hsa-miR-26b, hsa-miR-10a-5p, hsa-miR-99b, hsa-miR-146b, hsa-miR-24, hsa-miR-146a, hsa-miR-145, hsa-miR-25, hsa-miR-140-3p, or a combination thereof. In some embodiments, the miRNA signature of the EVs includes one or more functionally equivalent plant miRNAs.

In some embodiments, the miRNA signature of the EVs comprises miRNAs that are involved in cell proliferation. In some embodiments, the human miRNAs that are involved in cell proliferation comprise hsa-miR-100-5p, hsa-miR-125-5p, hsa-miR-24-3p, hsa-miR-146a-5p, hsa-miR-222-3p, hsa-miR-22-3p, hsa-miR-21-5p, hsa-let-7a-5p, hsa-miR-29a-3p, or a combination thereof. In some embodiments, the miRNA signature of the EVs includes one or more functionally equivalent plant miRNAs.

In some embodiments, the miRNA signature of the EVs comprises miRNAs that are involved in wound healing. In some embodiments, the human miRNAs that are involved in wound healing comprise hsa-miR-10b, hsa-miR-21, hsa-miR-146a, hsa-miR-10a, hsa-miR-146b, hsa-miR-155, or a combination thereof. In some embodiments, the miRNA signature of the EVs includes one or more functionally equivalent plant miRNAs.

In some embodiments, the miRNA signature of the EVs comprises miRNAs that are involved in angiogenesis. In some embodiments, the human miRNAs that are involved in angiogenesis comprise hsa-miR-143, hsa-miR-125-5p, hsa-miR-125a-5p, hsa-miR-26a-5p, hsa-miR-29a-3p, hsa-miR-222, hsa-miR-186-5p, hsa-miR-155-5p, hsa-miR-22-3p, hsa-miR-21-5p, hsa-miR-199a-3p, hsa-miR-92a-3p, hsa-miR-221, hsa-miR-27b, hsa-miR-181a-5p, hsa-let-7b, or a combination thereof. In some embodiments, the miRNA signature of the EVs includes one or more functionally equivalent plant miRNAs.

In some embodiments, the miRNA signature of the EVs comprises miRNAs that are involved in melanogenesis, pigmentation or both. In some embodiments, the human miRNAs that are involved in melanogenesis and pigmentation comprise hsa-miR-125, hsa-miR-21-5p, hsa-miR-148a-3p, hsa-miR-143-5p, hsa-miR-221-3p, hsa-miR-222, hsa-miR-25, hsa-miR-27a-3p, hsa-miR-21a-3p, or a combination thereof. In some embodiments, the miRNA signature of the EVs includes one or more functionally equivalent plant miRNAs.

In some embodiments, the miRNA signature of the EVs comprises miRNAs that are involved in photoaging, UV damage, or both. In some embodiments, the miRNAs that are involved in photoaging and UV damage comprise hsa-miR-125b, hsa-miR-21-5p, hsa-miR-23a, hsa-miR-146a, hsa-miR-145, hsa-miR-155, hsa-miR-21-3p, or a combination thereof. In some embodiments, the miRNA signature of the EVs includes one or more functionally equivalent plant miRNAs.

In some embodiments, the miRNA signature of the EVs comprises miRNAs that are involved in acne formation. In some embodiments, the human miRNAs that are involved in acne formation comprise hsa-miR-143, hsa-miR-146a-5p, or a combination thereof. In some embodiments, the miRNA signature of the EVs includes one or more functionally equivalent plant miRNAs.

In some embodiments, the miRNA signature of the EVs comprises miRNAs that are involved in hair growth, inhibition of hair loss, or both. In some embodiments, the human miRNAs that are involved in hair growth and inhibition of hair loss comprise hsa-miR-21-5p, hsa-miR-24, hsa-miR-26a-5p, hsa-miR-146a, hsa-let-7b, hsa-miR-101-3p, hsa-miR-181a-5p, hsa-miR-21-3p, hsa-miR-148b-3p, hsa-miR-481-5p, hsa-miR-103a-3p, or a combination thereof. In some embodiments, the miRNA signature of the EVs includes one or more functionally equivalent plant miRNAs.

In some embodiments, the miRNA signature comprises one or more of: hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, as described in Table 4.

TABLE 4
“Tuned” EV Cargo: miRNA Signature

| | | SEQ | |
|---|---|---|---|
| | | ID | |
| miRNA | Sequence | NO: | Significance |
| hsa-miR-100-5p | AACCCGUAGAUCCGAA | 126 | implicated in various cellular processes, |
| | CUUGUG | | including the regulation of apoptosis |
| hsa-miR-10b-5p | UACCCUGUAGAACCGA | 127 | characterized as tumorigenic and |
| | AUUUGUG | | progrowth, suggesting that its increased |
| | | | expression could contribute to altered |
| | | | cell growth and invasion properties |
| hsa-miR-143-3p | UGAGAUGAAGCACUGU | 128 | plays a significant role in the regulation |
| | AGCUC | | of various cellular processes, such as |
| | | | inflammation and apoptosis |
| hsa-miR-10a-5p | UACCCUGUAGAUCCGA | 129 | interacts with the 5′ untranslated regions |
| | AUUUGUG | | (UTRs) of mRNAs, particularly those |
| | | | with a terminal oligopyrimidine (TOP) |
| | | | motif, which can lead to both repression |
| | | | and activation of translation, suggesting |
| | | | a complex role in post-transcriptional |
| | | | regulation |
| hsa-miR-22-3p | AAGCUGCCAGUUGAAG | 130 | implicated in various cellular processes, |
| | AACUGU | | including the inflammatory response |
| hsa-miR-125b-5p | UCCCUGAGACCCUAAC | 131 | implicated in the pathophysiology of |
| | UUGUGA | | Alzheimer′s disease (AD) due to its |
| | | | dysregulated expression in the brains of |
| | | | individuals with AD |
| hsa-miR-21-5p | UAGCUUAUCAGACUGA | 132 | an oncogenic microRNA implicated in |
| | UGUUGA | | various cellular processes, including |
| | | | wound healing and cancer pathogenesis |
| hsa-miR-99b-5p | CACCCGUAGAACCGAC | 133 | implicated in the regulation of insulin- |
| | CUUGCG | | like growth factor 1 receptor (IGF1R) |
| | | | and may contribute to a coordinated |
| | | | shutdown of signal transduction |
| | | | pathways that block NF-κB pathways |
| hsa-miR-125a-5p | UCCCUGAGACCCUUUA | 134 | involved in the regulation of |
| | ACCUGUGA | | granulocytic differentiation by targeting |
| | | | Clec5a/MDL1 expression |
| hsa-let-7i-5p | UGAGGUAGUAGUUUGU | 135 | implicated in various biological |
| | GCUGUU | | processes and diseases, including sepsis |
| | | | and cancer. It is also found within |
| | | | extracellular vesicles (EVs), indicating |
| | | | its role in intercellular communication |
| hsa-miR-146b-5p | UGAGAACUGAAUUCCA | 136 | involved in the regulation of the NF-kB |
| | UAGGCUG | | signaling pathway, a key player in the |
| | | | control of immune responses and cell |
| | | | survival |
| hsa-miR-99a-5p | AACCCGUAGAUCCGAU | 137 | identified as a regulator of NOX4 |
| | CUUGUG | | expression by targeting the 3′-UTR |
| hsa-let-7f-5p | UGAGGUAGUAGAUUGU | 138 | implicated in cancer and DNA damage |
| | AUAGUU | | response pathways |
| hsa-miR-24-3p | UGGCUCAGUUCAGCAG | 139 | implicated in various biological |
| | GAACAG | | processes and diseases, with its |
| | | | recognition sequence identified in the |
| | | | 3′UTR of the human SLITRKI gene |
| hsa-let-7a-5p | UGAGGUAGUAGGUUGU | 140 | significant due to its well-defined CpG |
| | AUAGUU | | island, suggesting a role in gene |
| | | | regulation through methylation |
| hsa-miR-26a-5p | UUCAAGUAAUCCAGGA | 141 | regulates gene expression, acting |
| | UAGGCU | | primarily as a tumor suppressor in |
| | | | various cancers by inhibiting cell |
| | | | proliferation, migration, and invasion |
| | | | while promoting apoptosis |
| hsa-miR-148a-3p | UCAGUGCACUACAGAA | 142 | observed in immune cells from patients |
| | CUUUGU | | with lupus, implicating it in |
| | | | inflammatory dysfunction and |
| | | | conditions such as T cell autoreactivity |
| | | | and cytokine production |
| hsa-miR-1246 | AAUGGAUUUUUGGAGC | 143 | identified to have a molecular target in |
| | AGG | | cells; it is capable of binding to early B |
| | | | cytokine 1 (EBF1) at its 3′ untranslated |
| | | | region (3′UTR), suggesting a role in |
| | | | post-transcriptional regulation |
| hsa-miR-199b-3p | ACAGUAGUCUGCACAU | 144 | implicated in various biological |
| | UGGUUA | | processes and disease states. |
| hsa-miR-199a-3p | ACAGUAGUCUGCACAU | 145 | plays a key role in maintaining |
| | UGGUUA | | homeostasis, promoting cardiac |
| | | | regeneration, and acting as either an |
| | | | oncogene or tumor suppressor |
| | | | depending on the cancer type |
| hsa-miR-199a-5p | CCCAGUGUUCAGACUA | 146 | plays a key role in maintaining |
| | CCUGUUC | | homeostasis, promoting cardiac |
| | | | regeneration, and acting as either an |
| | | | oncogene or tumor suppressor |
| | | | depending on the cancer type |
| hsa-miR-23a-3p | AUCACAUUGCCAGGGA | 147 | involved in a variety of biological |
| | UUUCC | | processes, particularly those related to |
| | | | cell growth and survival |
| hsa-miR-92a-3p | UAUUGCACUUGUCCCG | 148 | regulates gene expression and is |
| | GCCUGU | | frequently upregulated in cancers, |
| | | | promoting tumor cell migration, |
| | | | invasion, and proliferation by inhibiting |
| | | | apoptosis |
| hsa-miR-127-3p | UCGGAUCCGUCUGAGC | 149 | implicated in the regulation of |
| | UUGGCU | | inflammatory responses, targeting IgG |
| | | | FcγRI (CD64) and modulating cytokine |
| | | | release by macrophages during lung |
| | | | inflammation |
| hsa-miR-152-3p | UCAGUGCAUGACAGAA | 150 | implicated in liver epithelial-to- |
| | CUUGG | | mesenchymal transition (EMT) as it can |
| | | | be competitively bound by the long |
| | | | non-coding RNA plasmacytoma variant |
| | | | translocation 1 (PVT1), which indicates |
| | | | a potential role for MIR152 in liver |
| | | | EMT processes |
| hsa-miR-146a-5p | UGAGAACUGAAUUCCA | 151 | implicated in the immune response as it |
| | UGGGUU | | is differentially expressed between |
| | | | naive and memory T cells and is |
| | | | induced by TCR stimulation |
| hsa-let-7g-5p | UGAGGUAGUAGUUUGU | 152 | implicated in the regulation of key stem |
| | ACAGUU | | cell factors and differentiation processes |
| | | | such as adipogenesis and osteogenesis |
| hsa-miR-191-5p | CAACGGAAUCCCAAAA | 153 | identified as notably expressed across |
| | GCAGCUG | | various tumor types and is considered a |
| | | | key miRNA ortholog among those that |
| | | | are mutually abundant in these tumors |
| hsa-miR-29a-3p | UAGCACCAUCUGAAAU | 154 | involved in various biological |
| | CGGUUA | | processes, including matrix remodeling |
| | | | and fibrosis, as indicated by its |
| | | | formation being primed by myocardin |
| hsa-miR-151a-3p | CUAGACUGAAGCUCCU | 155 | Involved in the regulation of genes that |
| | UGAGG | | favor angiogenesis |
| hsa-let-7b-5p | UGAGGUAGUAGGUUGU | 156 | increased during oxygen exposure, |
| | GUGGUU | | leading to oxidative stress and cellular |
| | | | senescence in the choroid and retinal |
| | | | pigment epithelium via the p53-let-7b- |
| | | | IGF-1R pathway |
| hsa-miR-199b-5p | CCCAGUGUUUAGACUA | 157 | implicated in various biological |
| | UCUGUUC | | processes and disease states |
| hsa-miR-145-3p | GGAUUCCUGGAAAUAC | 158 | functions primarily as a tumor |
| | UGUUCU | | suppressor, regulating gene expression |
| | | | post-transcriptionally to inhibit cancer |
| | | | progression |
| hsa-miR-221-3p | AGCUACAUUGUCUGCU | 159 | implicated in various cellular processes, |
| | GGGUUUC | | including apoptosis and autophagy, as |
| | | | well as in the pathogenesis of multiple |
| | | | hyperlipidemia-related diseases (HRDs) |
| | | | and cancer |
| hsa-miR-222-3p | AGCUACAUCUGGCUAC | 160 | implicated in various cellular processes, |
| | UGGGU | | including adipogenesis and vascular |
| | | | smooth muscle cell (vSMC) |
| | | | proliferation |
| hsa-miR-27b-3p | UUCACAGUGGCUAAGU | 161 | identified as a regulator of VEGFC |
| | UCUGC | | expression and has been associated with |
| | | | the suppression of tumor growth and |
| | | | metastasis in various cancers including |
| | | | gastric cancer, bladder cancer, and |
| | | | hepatocellular carcinoma |
| hsa-miR-30d-5p | UGUAAACAUCCCCGAC | 162 | plays a significant role in the regulation |
| | UGGAAG | | of human adipocyte development |
| hsa-miR-25-3p | CAUUGCACUUGUCUCG | 163 | acts as an oncogene in various cancers |
| | GUCUGA | | (e.g., prostate, pancreatic, colorectal) by |
| | | | promoting cell proliferation, migration, |
| | | | and invasion |
| hsa-miR-101-3p | UACAGUACUGUGAUAA | 164 | correlated with Th2 cell-related genes |
| | CUGAA | | and is implicated in the regulation of |
| | | | immune cell function |
| hsa-miR-221-5p | ACCUGGCAUACAAUGU | 165 | implicated in various cellular processes, |
| | AGAUUU | | including apoptosis and autophagy, as |
| | | | well as in the pathogenesis of multiple |
| | | | hyperlipidemia-related diseases (HRDs) |
| | | | and cancer |
| hsa-miR-423-3p | AGCUCGGUCUGAGGCC | 166 | regulates gene expression and acts |
| | CCUCAGU | | primarily as an oncogene in various |
| | | | cancers, promoting tumor cell |
| | | | proliferation, migration, invasion, and |
| | | | drug resistance |
| hsa-miR-186-5p | CAAAGAAUUCUCCUUU | 167 | acts as a tumor suppressor in various |
| | UGGGCU | | cancers (including prostate and lung |
| | | | cancer) by inhibiting tumor growth, |
| | | | invasion, and migration |
| hsa-miR-30a-5p | UGUAAACAUCCUCGAC | 168 | binds to the 3′ untranslated regions |
| | UGGAAG | | (UTRs) of specific messenger RNAs |
| | | | (mRNAs) to inhibit their translation, |
| | | | generally functioning as a tumor |
| | | | suppressor and regulator of cell |
| | | | autophagy and motility. |
| hsa-miR-151a-5p | UCGAGGAGCUCACAGU | 169 | identified as a mitochondria-related |
| | CUAGU | | miRNA that can reduce cellular ATP |
| | | | production, impacting cellular energy |
| | | | metabolism |
| hsa-miR-151b | UCGAGGAGCUCACAGU | 170 | regulates gene expression at the post- |
| | CU | | transcriptional level, influencing |
| | | | processes like cell differentiation, |
| | | | proliferation, and apoptosis |
| hsa-miR-27a-3p | UUCACAGUGGCUAAGU | 171 | involved in regulating cell |
| | UCCGC | | differentiation, such as in adipogenesis |
| | | | (fat cell formation) and as a negative |
| | | | regulator of laryngeal cancer cell |
| | | | differentiation |
| hsa-miR-224-5p | UCAAGUCACUAGUGGU | 172 | plays a significant role in various |
| | UCCGUUUAG | | biological processes and has been |
| | | | linked to diseases such as cancer and |
| | | | autoimmune disorders |
| hsa-miR-181a-5p | AACAUUCAACGCUGUC | 173 | implicated in various cellular processes, |
| | GGUGAGU | | including the modulation of cancer |
| | | | progression and glucose metabolism |
| hsa-miR-28-3p | CACUAGAUUGUGAGCU | 174 | acts as a crucial post-transcriptional |
| | CCUGGA | | regulator in the immune system, acting |
| | | | as a tumor suppressor in lymphomas |
| | | | and modulating immune responses. It |
| | | | influences cell proliferation, Apoptosis |
| | | | (cell death), and differentiation |
| hsa-miR-320a | GCCUUCUCUUCCCGGU | 175 | acts as a significant regulator of |
| | UCUUCC | | immune, inflammatory, and, in many |
| | | | cases, tumor-suppressive processes. It |
| | | | influences immune responses by |
| | | | modulating how cells react to stress, |
| | | | inflammation, and infections, often by |
| | | | targeting signaling pathways like |
| | | | MAPK, PI3K-AKT, and mTOR |
| hsa-miR-378a-3p | ACUGGACUUGGAGUCA | 176 | acts as a regulator in the immune |
| | GAAGGC | | system and tumor microenvironment, |
| | | | typically influencing inflammatory |
| | | | responses, cell proliferation, and |
| | | | angiogenesis |
| hsa-miR-155-5p | UUAAUGCUAAUCGUGA | 177 | implicated in the regulation of various |
| | UAGGGGUU | | cellular processes, including immune |
| | | | response and oncogenesis |
| hsa-miR-193a-5p | UGGGUCUUUGCGGGCG | 178 | modulates the tumor microenvironment |
| | AGAUGA | | (TME) by enhancing T-cell-mediated |
| | | | immune responses, triggering |
| | | | immunogenic cell death, and inhibiting |
| | | | tumor progression |
| hsa-let-7c-5p | UGAGGUAGUAGGUUGU | 179 | key regulator of immune responses, |
| | AUGGUU | | typically suppressing inflammation by |
| | | | targeting pro-inflammatory cytokines |
| | | | like IL-6, IFN-?, and TNF-a. It plays a |
| | | | critical role in controlling immune |
| | | | effector cells, regulating apoptosis (cell |
| | | | death), and acting as a tumor |
| | | | suppressor, with decreased levels often |
| | | | associated with chronic disease severity |
| hsa-miR-21-3p | CAACACCAGUCGAUGG | 180 | implicated in various cellular processes, |
| | GCUGU | | including wound healing and cancer |
| | | | pathogenesis |
| hsa-miR-196a-5p | UAGGUAGUUUCAUGUU | 181 | located within the homeobox (HOX) |
| | GUUGGG | | gene clusters on human chromosome |
| | | | 17q21.32 and plays a role in gene |
| | | | expression regulation |
| hsa-miR-148b-3p | UCAGUGCAUCACAGAA | 182 | a post-transcriptional regulator in the |
| | CUUUGU | | immune system, often modulating |
| | | | inflammatory responses and tumor |
| | | | immunity. It controls macrophage |
| | | | polarization toward an M2 phenotype |
| | | | (pro-tumor), impacts autoimmune |
| | | | conditions and chronic inflammation, |
| | | | and plays a key role in immune |
| | | | surveillance, with its downregulation |
| | | | often associated with disease states |
| hsa-miR-409-5p | AGGUUACCCGAGCAAC | 183 | implicated in various biological |
| | UUUGCAU | | processes and diseases, including its |
| | | | deregulation in gastrointestinal stromal |
| | | | tumors (GISTs), where it is expressed |
| | | | higher in intestinal compared to gastric |
| | | | tumors |
| hsa-miR-92b-3p | UAUUGCACUCGUCCCG | 184 | implicated in various biological |
| | GCCUCC | | processes including the attenuation of |
| | | | inflammatory responses and autophagy |
| | | | by targeting TRAF3 and the MKK3- |
| | | | p38 pathway in acute pancreatitis |
| hsa-miR-140-3p | UACCACAGGGUAGAAC | 185 | a critical immune modulator, primarily |
| | CACGG | | by suppressing inflammatory responses, |
| | | | reducing pro-inflammatory cytokine |
| | | | production (e.g., IL-1β, IL-6, TNF-a), |
| | | | and maintaining immune cell |
| | | | homeostasis |
| hsa-miR-486-5p | UCCUGUACUGAGCUGC | 186 | plays a complex role in regulating |
| | CCCGAG | | immune responses, particularly in |
| | | | inflammation, cell differentiation, and |
| | | | tumor-immune interactions |
| hsa-miR-30e-5p | UGUAAACAUCCUUGAC | 187 | a regulator of the innate immune |
| | UGGAAG | | response |
| hsa-miR-615-3p | UCCGAGCCUGGGUCUC | 188 | plays a complex role in immune |
| | CCUCUU | | regulation, primarily acting to suppress |
| | | | inflammatory responses and modulate |
| | | | the activity of immune cells like Natural |
| | | | Killer (NK) cells and macrophages |
| hsa-miR-409-3p | GAAUGUUGCUCGGUGA | 189 | implicated in various biological |
| | ACCCCU | | processes and diseases, including its |
| | | | deregulation in gastrointestinal stromal |
| | | | tumors (GISTs), where it is expressed |
| | | | higher in intestinal compared to gastric |
| | | | tumors |
| hsa-miR-501-3p | AAUGCACCCGGGCAAG | 190 | regulates gene expression, frequently |
| | GAUUCU | | acting as an oncogene in cancers |
| | | | (promoting proliferation, migration, and |
| | | | invasion) or as a biomarker for |
| | | | neurodegenerative diseases |
| hsa-miR-192-5p | CUGACCUAUGAAUUGA | 191 | implicated in a variety of pathological |
| | CAGCC | | conditions, including diabetes and heart |
| | | | failure |
| hsa-miR-103a-3p | AGCAGCAUUGUACAGG | 192 | acts as an oncogene, promoting tumor |
| | GCUAUGA | | growth, proliferation, and invasion in |
| | | | various cancers, including lung, breast, |
| | | | colorectal, and liver cance |
| hsa-miR-26b-5p | UUCAAGUAAUUCAGGA | 193 | implicated in the regulation of gene |
| | UAGGU | | expression, specifically involved in the |
| | | | repression of EZH2, an enzyme that |
| | | | contributes to gene silencing through |
| | | | histone methylation |
| hsa-miR-1271-5p | CUUGGCACCUAGCAAG | 194 | identified as a tumor suppressor |
| | CACUCA | | microRNA with a significant role in |
| | | | hepatocellular carcinoma (HCC) |
| hsa-miR-500a-3p | AUGCACCUGGGCAAGG | 195 | functions as an oncogene in various |
| | AUUCUG | | cancers-such as hepatocellular |
| | | | carcinoma, colorectal, and breast |
| | | | cancer-by promoting cell |
| | | | proliferation, migration, and invasion |
| hsa-miR-145-5p | GUCCAGUUUUCCCAGG | 196 | plays a role in regulating stem cell |
| | AAUCCCU | | pluripotency |
| hsa-miR-484 | UCAGGCUCAGUCCCCU | 197 | regulates immune responses, |
| | CCCGAU | | inflammation, and apoptosis, frequently |
| | | | acting as a, anti-inflammatory |
| | | | modulator that reduces tissue injury |
| hsa-miR-125b-2- | UCACAAGUCAGGCUCU | 198 | promotes macrophage activation, |
| 3p | UGGGAC | | enhances type I interferon production, |
| | | | and regulates T-cell proliferation |
| hsa-miR-361-5p | UUAUCAGAAUCUCCAG | 199 | plays a regulatory role in immune |
| | GGGUAC | | responses, inflammation, and cell |
| | | | survival, often acting as a modulator of |
| | | | pro-inflammatory signaling pathways |
| | | | and apoptosis |
| hsa-miR-503-5p | UAGCAGCGGGAACAGU | 200 | regulatory microRNA that modulates |
| | UCUGCAG | | immune responses, particularly by |
| | | | inhibiting inflammation, promoting |
| | | | monocytic differentiation, and |
| | | | influencing tumor immune escape |
| hsa-miR-379-5p | UGGUAGACUAUGGAAC | 201 | acts as an immune regulator, primarily |
| | GUAGG | | by enhancing T cell function and |
| | | | reducing exhaustion, particularly in |
| | | | cancer settings |

In some embodiments, the tuned cargo of the EVs comprises a protein signature. In some embodiments, the protein signature comprises one or more proteins involved in cell growth and differentiation, transcriptional regulation, cellular signaling, cell cycle and proliferation, inflammatory responses, extracellular matrix function, heat stress responses, EV function, or a combination thereof.

In some embodiments, the protein signature comprises one or more proteins involved in cell growth and differentiation. In some embodiments, the one or more proteins involved in cell growth and differentiation comprise BMP2, BMP4, TGFB2, IGF1, KITLG, NOG, LEP, WNT5A, or a combination thereof.

In some embodiments, the protein signature comprises one or more proteins involved in transcriptional regulation. In some embodiments, the one or more proteins involved in transcriptional regulation comprise SOX2, TCF4, AR, HEY1, or a combination thereof.

In some embodiments, the protein signature comprises one or more proteins involved in cellular signaling. In some embodiments, the one or more proteins involved in cellular signaling comprise BMP2, BMP4, TGFB2, IGF1, KITLG, IL1B, NOG, STAT5A, STAT6, AXIN2, SFRP1, WNT5A, LEP, or a combination thereof.

In some embodiments, the protein signature comprises one or more proteins involved in cell cycle and proliferation. In some embodiments, the one or more proteins involved in cell cycle and proliferation comprise CDKN2A, MKI67, or a combination thereof.

In some embodiments, the protein signature comprises one or more proteins involved in inflammatory responses. In some embodiments, the one or more proteins involved in inflammatory responses comprise IL1B.

In some embodiments, the protein signature comprises one or more proteins involved in extracellular matrix function. In some embodiments, the one or more proteins involved in extracellular matrix function comprise DCN.

In some embodiments, the protein signature comprises one or more proteins involved in heat stress responses. In some embodiments, the one or more proteins involved in heat stress responses comprise HSP27, HSP60, HSP70, HSP90, or a combination thereof.

In some embodiments, the protein signature comprises one or more proteins involved in cellular signaling. [See Termini and Gilette, Frontiers Cel & Devel. Bio. (2017) 5:34]. In some embodiments, the one or more proteins involved in cellular signaling comprise CD9, CD63, CD81, CD82, or a combination thereof.

In some embodiments, the protein signature comprises one or more of BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82 and WNT5A.

### Functional Properties of the Compositions of the Present Disclosure

In some embodiments, administration of the composition comprising a population of EVs comprising a “tuned” cargo modulates the biological activity of recipient cells. In some embodiments, the recipient cells comprise cells that contact the population of EVs comprising a “tuned” cargo. In some embodiments, the recipient cells are human cells. Nonlimiting examples of human cells include epidermal cells (e.g., keratinocytes, corneocytes, melanocytes, Langerhans cells, Merkel cells), dermal cells (e.g., fibroblasts, dendritic cells, mast cells, macrophages, lymphocytes), hair follicle-associated cells (e.g., dermal papilla cells, dermal sheath cells hair follicle stem cells), vascular and endothelial cells, immune cells (e.g., macrophages, monocytes, lymphocytes, neutrophils, dendritic cells, mast cells, NK cells), adipose cells, mucosal and genital tissue cells, or a combination thereof.

In some embodiments, administration of the composition comprising a population of EVs comprising a “tuned” cargo modulates gene expression in the recipient cell. In some embodiments, modulating gene expression comprises differential gene expression if at least one gene compared to a nonrecipient cell (i.e., a cell that does not contact the population of EVs comprising a “tuned” cargo). According to some embodiments, the differential gene expression results in increased expression (i.e., overexpression) of at least one gene. In some embodiments, the differential gene expression results in reduced expression of at least one gene. Examples of assessing gene expression include but are not limited to RNA sequencing (RNA-seq) and quantitative PCR (qPCR).

According to some embodiments, the increased expression of at least one gene is by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to nonrecipient cell control.

According to some embodiments, the reduced expression of at least one gene is by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to a nonrecipient cell control.

In some embodiments, administration of the composition comprising a population of EVs comprising a “tuned” cargo modulates collagen production in the recipient cell. Nonlimiting examples of techniques to measure the production of collagen include qPCR, western blot, immunohistochemistry, immunofluorescence, ELISA, and flow cytometry.

In some embodiments, administration of the composition comprising a population of EVs comprising a “tuned” cargo modulates cytokine production in the recipient cell. Non-limiting examples of cytokines include e.g., IL-1, IL-2, IL-3, IL-4, IL-5, IL-6, IL-7, IL-8, IL-9, IL-10, IL-11, IL-12/IL-23 P40, IL13, IL-15, IL-15/IL15-RA, IL-17, IL-18, IL-21, IL-23, TGF-β, IFN-γ, GM-CSF, Groα, MCP-1 and TNF-α. Nonlimiting examples of techniques to monitor cytokine production include flow cytometry, ELISA, MagPix™ multiplex assay, and ELISpot™.

In some embodiments, administration of the composition comprising a population of EVs comprising a “tuned” cargo modulates cell proliferation in the recipient cell. In some embodiments, cell proliferation can be assessed using a cell proliferation assay, including but not limited to, a 5-bromo-2′-deoxyuridine (BrdU) enzyme immunoassay, a carboxyfluorescein (CFSE) assay, or a Ki-67 assay.

In some embodiments, administration of the composition comprising a population of EVs comprising a “tuned” cargo modulates extracellular matrix remodeling in the recipient cell. In some embodiments, extracellular matrix remodeling is measured by assessing the production of collagen, elastin, hyaluronic acid, fibronectin, laminin, or a combination thereof. Nonlimiting examples of techniques to measure the production of collagen, elastin, hyaluronic acid, fibronectin, laminin, or a combination thereof include qPCR, western blot, immunohistochemistry, immunofluorescence, ELISA, and flow cytometry.

In some embodiments, administration of the composition comprising a population of EVs comprising a “tuned” cargo modulates oxidative stress in the recipient cell. In some embodiments, administration of the composition comprising a population of EVs comprising a “tuned” cargo decreases the amount of reactive oxygen species (ROS) in the recipient cell. Nonlimiting examples of ROS include superoxide anion, hydrogen peroxide, hydroxyl radical, singlet oxygen, hydroperoxyl radical, peroxyl radical, alkoxyl radical, and hypochlorous acid. Nonlimiting examples of techniques to measure oxidative stress includes measuring DNA/RNA damage by detecting 8-hydroxyguanosine (i.e., an oxidized nucleoside derivative of guanosine, used to measure RNA damage), 8-hydroxydeoxyguanosine (i.e., an oxidized nucleoside derivative of guanosine, used to measure DNA damage), and double strand breaks, measuring lipid damage by measuring lipid peroxidation, and measuring protein damage by measuring protein modifications (e.g., oxidation, protein carbonyl content).

In some embodiments, administration of the composition comprising a population of EVs comprising a “tuned” cargo modulates cell signaling in the recipient cell. In some embodiments the signaling pathway is associated with inflammation, immune dysfunction, immune regulation, skin structure and function, including aging, UV-induced photoaging, epigenetics, cancer, cell cycle regulation, DNA repair, cell proliferation, or a combination thereof.

In some embodiments, the signaling pathway is a PI3K/AKT/mTOR pathway, a MAPK pathway, an IGF-1R pathway, a LPR1 pathway, a WNT pathway, a JAK/STAT pathway, a leptin pathway, a VEGFA/VEGFR1 pathway, a BMP pathway, cytokine signaling, interleukin signaling or a combination thereof.

### Formulations of the Present Disclosure

Compositions of the present disclosure may be formulated using technology described in Remington's Pharmaceutical Sciences, 18th or 19th editions, published by the Mack Publishing Company of Easton, Pennsylvania.

The compositions of the described invention may be usefully formulated in cosmetic, cosmeceutical, and general skincare compositions, as well as in pharmaceutical compositions. According to some embodiments, the composition is a cosmetic composition comprising a cosmetic amount of a population of EVs comprising a “tuned” cargo, and a cosmetically acceptable carrier. According to some embodiments, the composition is a cosmeceutical composition comprising a cosmeceutical amount of a population of EVs comprising a “tuned” cargo, and a cosmeceutical carrier.

According to some embodiments, a nutraceutical composition providing health benefits in addition to its basic nutritional value, and not intended to diagnose, treat, cure, or prevent any disease comprises a nutraceutical amount of a population of EVs comprising a “tuned” cargo and an acceptable carrier.

According to some embodiments, where the intent is to diagnose, treat, cure, or prevent a disease, the composition is a pharmaceutical composition comprising a pharmaceutical amount of a population of EVs comprising a “tuned” cargo and a pharmaceutically acceptable carrier.

### Carriers and Excipients

Suitable acceptable carriers are described in the CTFA International Cosmetic Ingredient Dictionary and Handbook, 8th edition, edited by Wenninger and Canterbery, (The Cosmetic, Toiletry, and Fragrance Association, Inc., Washington, D. C, 2000), which is herein incorporated by reference. It will be understood that cosmetically acceptable carriers, cosmeceutically acceptable carriers, and pharmaceutically acceptable carriers are similar, if not often identical, in nature,

Exemplary carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. According to some embodiments, the pharmaceutically acceptable carrier is sterile and pyrogen-free water. According to some embodiments, the pharmaceutically acceptable carrier is Ringer's Lactate, sometimes known as lactated Ringer's solution.

In some embodiments, the composition includes pharmaceutically acceptable salts. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid and phosphoric acid and such organic acids as oxalic acid, maleic acid, succinic acid and citric acid.

Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made

Wetting agents, emulsifiers and lubricants, such as sodium lauryl sulfate and magnesium stearate, as well as coloring agents, release agents, coating agents, sweetening, flavoring and perfuming agents, preservatives and antioxidants may also be present in the compositions.

Nonlimiting examples of pharmaceutically acceptable antioxidants include water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Nonlimiting examples of suitable carriers, excipients, and diluents include lactose, dextrose, sucrose, sorbitol, mannitol, starches, gum acacia, calcium phosphate alginates, calcium silicate, microcrystalline cellulose, polyvinylpyrrolidone, cellulose, tragacanth, gelatin, syrup, methyl cellulose, methyl- and propylhydroxybenzoates, talc, magnesium stearate, water, and mineral oil.

In some embodiments, the pharmaceutically acceptable carrier comprises a surfactant. The formulations may additionally include lubricating agents, wetting agents, emulsifying and suspending agents, preserving agents, sweetening agents or flavoring agents. The compositions may be formulated so as to provide quick, sustained, or delayed release of the active ingredient after administration to the subject.

As is widely recognized in the art, since the pH of human skin is 5.5, to avoid irritation, compositions for topical skin application should have a pH value of between pH 4.0 and pH 7.0, inclusive. In some embodiments, the pH of the composition is between pH 5.0 and pH 7.0, inclusive. In some embodiments, the pH is about 5.5. Hence, a pH adjusting composition is typically added to bring the pH of the composition to the desired value. The compositions of the described invention therefore can be formulated to have a pH value of about 7.0. Suitable pH adjusting agents include, for example, but are not limited to, one or more adipic acids, glycines, citric acids, calcium hydroxides, magnesium aluminometasilicates, buffers or any combinations thereof.

### Routes of Administration

According to some embodiments, the present disclosure provides topical compositions comprising a population of EVs comprising a “tuned” cargo and a carrier. Topical administration may also involve the use of transdermal administration such as transdermal patches or iontophoresis devices which are prepared according to techniques and procedures well known in the art. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation.

Patches suitable for use in the present invention include, but are not limited to, (1) the matrix patch; (2) the reservoir patch; (3) the multi-laminate drug-in-adhesive patch; and (4) the monolithic drug-in-adhesive patch; TRANSDERMAL AND TOPICAL DRUG DELIVERY SYSTEMS, pp. 249-297 (Tapash K. Ghosh et al. eds., 1997), hereby incorporated herein by reference. These patches are well known in the art and generally available commercially.

Dissolvable microneedles comprising patches composed of microscopic cones and often composed of sugar and salt solutions can deliver compositions directly to the dermis and the immune cells that reside there. The composition is added to the sugar and salt solution and dried; once the microneedles are applied to the skin, the moisture in a patient's skin can dissolve the microneedles and absorb the composition components.

The described invention relates to all routes of administration including intramuscular, subcutaneous, sublingual, intravenous, intraperitoneal, intranasal, intratracheal, intravaginal, transvaginal, topical, intradermal, intramucosal, intracavernous, intrarectal, into a sinus, gastrointestinal, intraductal, intrathecal, intraventricular, intrapulmonary, into an abscess, intraarticular, subpericardial, into an axilla, into the pleural space, intrabuccal, transmucosal, transdermal, via inhalation, via nebulizer, and via subcutaneous injection. Alternatively, the plant-derived exosome composition may be introduced by various means into cells that are removed from the individual. Such means include, for example, microprojectile bombardment, via liposomes or via other nanoparticle device.

Transmucosal drug administration refers to the absorption of drug through the mucosal epithelium into the systemic circulation. Intranasal, oral transmucosal and rectal routes are the major transmucosal routes. Mucus is a universal delivery barrier at the mucosal surface, and drugs are usually absorbed through the mucosa into the systemic circulation through either the transcellular or paracellular route. They typically have low molecular weight (<500 Da) with a high log P value (>2.0), where P is the octanol-water partition coefficient. The higher the log P, the more lipophilic the drug is [Lam, J K W., et al. Adv. Drug Deliv. Rev. (2020) 160: 234-43]. Only a small number of drugs exhibit the desirable physicochemical properties to cross the mucosal epithelium effectively. In order to enhance drug absorption, penetration enhancers (or permeation enhancers) are commonly investigated in the development of transmucosal formulations to overcome the epithelial barrier, especially in intranasal, buccal, and to a lesser extent, rectal formulations.

Specific modes of administration will depend on the indication. The selection of the specific route of administration and the dose regimen is to be adjusted or titrated by the clinician according to methods known to the clinician in order to obtain the optimal clinical response. The amount of active agent to be administered is that amount sufficient to provide the intended benefit of treatment. The dosage to be administered will depend on the characteristics of the subject being treated, e.g., the particular mammal or human treated, age, weight, health, types of concurrent treatment, if any, and frequency of treatments, and can be easily determined by one of skill in the art (e.g., by the clinician).

Ocular formulations are intended to be applied on the anterior surface (topical route) of the eye, delivered intraocularly (inside the eye), periocularly (subtenon or juxtascleral), or in combination with ocular devices. Ocular dosage forms may be liquid, semi-solid, solid, or mixed. Liquid dosages include drops, suspension, and emulsion. Eye drops represent more than 95% of the marketed ocular products and are used for delivering the medication into the anterior part of the eye but with short residence time. Ocular suspensions and emulsions have the ability to deliver hydrophobic drugs but may lead to blurred vision. Ocular gels and ointments (semi-solid) may significantly enhance residence time. Solid dosage forms may be used to deliver water-sensitive drugs (powder), provide zero order release, or to sustain residence time (therapeutic contact lens). Effective ocular absorption necessitates appropriate corneal penetration along with effective precorneal residence time, so as to reach and preserve an acceptable drug concentration with the minimum quantity of the active therapeutic constituent. There are many ocular barriers such as tear film, corneal, conjunctival, and blood-ocular barriers that hinder their therapeutic efficacy. Because conventional eye drops are wasted by blinking and tear flow, their bioavailability is minimized to less than 5%. The cornea is composed of epithelium, stroma, and endothelium. The corneal epithelium allows only the passage of small and lipophilic drug. However, the corneal stroma allows the passage of hydrophilic drugs. The corneal endothelium conserves the transparency of the cornea and affords selective entry for hydrophilic drugs and macromolecules into the aqueous humor. The conjunctiva provides a minor impact to drug absorption compared to the cornea, though certain macromolecular nanomedicines, peptides, and oligonucleotides penetrate to the deep layers of the eye absolutely through these tissues [Ahmed, S., et al. AAPS PharmSciTech (2023) 24: article 66].

Intracameral injections involve injection of antibiotic directly into the anterior segment of the eyeball or in the vitreous cavity. It is done usually subsequent to cataract surgery to avoid endophthalmitis initiated by a contagion of the eye that can occur after cataract surgery. Intravitreal injection is a delivery of medicine into the vitreous that is close to the retina at the back of the eye. Juxtascleral injections are used for treatment of some posterior part complaints that cannot be handled through conventional topical route. It is used for the treatment of cystoid macula edema, trauma, and diabetic-related conditions. Retrobulbar route involves the injection of a needle through the eyelid and orbital fascia to deliver the medication behind the globe into the retrobulbar space. Subconjunctival injection is frequently used in cases of very low drug penetration into the anterior part of the eye after topical administration. Subconjunctival injection of steroids fabricated as PEGylated liposome for handling of uveitis showed sustained anti-inflammatory activity and targeting the required ocular tissue [Ahmed, S., et al. AAPS PharmSciTech (2023) 24: article 66].

### Dosage Forms

Formulations containing the population of EVs comprising a “tuned” cargo and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels, jellies, and foams; and parenteral dosage forms which include, but are not limited to, solutions, suspensions, emulsions, and dry powder; comprising an effective amount of a polymer or copolymer of the described invention.

The active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. See, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980).

The compositions of the described invention may be in a form suitable for oral use, for example, as tablets, troches, lozenges, aqueous or oily suspensions, sprays, dispersible powders or granules, emulsions, hard or soft capsules or syrups or elixirs. Compositions intended for oral use may contain one or more agents selected from the group consisting of sweetening agents, flavoring agents, coloring agents, and preserving agents in order to provide pharmaceutically elegant and palatable preparations.

Tablets may contain the active ingredient(s) in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They also may be coated for controlled release.

Compositions of the present disclosure also may be formulated for oral use as hard gelatin capsules, where the active ingredient(s) is(are) mixed with an inert solid diluent, for example, calcium carbonate, calcium phosphate or kaolin, or soft gelatin capsules wherein the active ingredient(s) is (are) mixed with water or an oil medium, for example, peanut oil, liquid paraffin, or olive oil.

The compositions of the present disclosure may be formulated as aqueous suspensions wherein the active ingredient(s) is (are) in admixture with excipients suitable for the manufacture of aqueous suspensions. Such excipients are suspending agents, for example, sodium carboxymethylcellulose, methylcellulose, hydroxy-propylmethylcellulose, sodium alginate, polyvinylpyrrolidone, gum tragacanth, and gum acacia; dispersing or wetting agents may be a naturally-occurring phosphatide such as lecithin, or condensation products of an alkylene oxide with fatty acids, for example, polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions also may contain one or more coloring agents, one or more flavoring agents, and one or more sweetening agents, such as sucrose or saccharin.

Compositions of the present disclosure may be formulated as oily suspensions by suspending the active ingredient in a vegetable oil, for example arachis oil, olive oil, sesame oil or coconut oil, or in a mineral oil, such as liquid paraffin. The oily suspensions may contain a thickening agent, for example, beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Compositions of the present invention may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients, for example, sweetening, flavoring and coloring agents may also be present.

Compositions of the invention also may be formulated as a beverage or as an additive to a beverage, where the term “beverage” refers to any non-alcoholic flavored carbonated drink, soda water, non-alcoholic still drinks, diluted fruit or vegetable juices whether sweetened or unsweetened, seasoned or unseasoned with salt or spice, or still or carbonated mineral waters used as a drink. The term “additive” as used herein refers to any substance the intended use of which results, or may reasonably be expected to result, directly or indirectly, in its becoming a component or otherwise affecting the characteristics of any beverage. In some embodiments, the beverage is a flavored carbonated beverage. In some embodiments, the beverage is a flavored non-carbonated beverage. In some embodiments, the beverage is a natural fruit beverage. The beverage also may contain one or more coloring agents, one or more flavoring agents, one or more sweetening agents, one or more antioxidant agents, and one or more preservatives.

Compositions of the invention also may be in the form of oil-in-water emulsions. The oily phase may be a vegetable oil, for example, olive oil or arachis oil, or a mineral oil, for example a liquid paraffin, or a mixture thereof. Suitable emulsifying agents may be naturally occurring gums, for example, gum acacia, naturally occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate. The emulsions also may contain sweetening and flavoring agents.

Compositions of the invention also may be formulated as syrups and elixirs. Syrups and elixirs may be formulated with sweetening agents, for example, glycerol, propylene glycol, sorbitol or sucrose. Such formulations also may contain a demulcent, a preservative, and flavoring and coloring agents. Demulcents are protective agents employed primarily to alleviate irritation, particularly mucous membranes or abraded tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Preparations that can be used orally include, but are not limited to, push-fit capsules made of gelatin, as well as soft, scaled capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

The compositions of the present disclosure may be in the form of a sterile injectable aqueous or oleaginous suspension. The term “parenteral” as used herein includes subcutaneous injections, intravenous, intramuscular, intra-articular, intracisternal injection, or infusion techniques. Injectable preparations, such as sterile injectable aqueous or oleaginous suspensions, solutions or emulsions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. The parenteral composition can be formulated, for example, as a solution, suspension, emulsion or lyophilized powder in association with a pharmaceutically acceptable parenteral vehicle. Among the acceptable vehicles and solvents that may be employed are water, Ringer's solution, and isotonic sodium chloride solution. In addition, sterile, fixed oils are conventionally employed as a solvent or suspending medium. For parenteral application, exemplary vehicles consist of solutions, preferably oily or aqueous solutions, as well as suspensions, emulsions, or implants.

Aqueous suspensions may contain substances which increase the viscosity of the suspension and include, for example, sodium carboxymethyl cellulose, sorbitol and/or dextran. Optionally, the suspension may also contain stabilizers. Formulations for injection can be presented in unit dosage form, e.g., in ampoules or in multi-dose containers, with an added preservative. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques.

The compositions of the present disclosure may be in the form of a dispersible dry powder for pulmonary delivery. Dry powder compositions may be prepared by processes such as lyophilization and jet milling as disclosed in International Patent Publication No. WO 91/16038 and as disclosed in U.S. Pat. No. 6,921,527, the disclosures of which are incorporated by reference. The composition of the present invention is placed within a suitable dosage receptacle in an amount sufficient to provide a subject with a unit dosage treatment. The dosage receptacle is one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. Nos. 4,227,522; 4,192,309; and 4,105,027. Suitable containers also include those used in conjunction with Glaxo's Ventolin® Rotohaler™ brand powder inhaler or Fison's Spinhaler® brand powder inhaler. Another suitable unit-dose container which provides a superior moisture barrier is formed from an aluminum foil plastic laminate. The pharmaceutical-based powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler® (U.S. Pat. Nos. 4,627,432; 4,811,731; and 5,035,237). All of these references are incorporated herein by reference.

Administration by inhalation or insufflation (either through the mouth or through the nose, respectively) may involve an inhalation delivery device or a solid particulate therapeutic aerosol generator. According to some embodiments, the solid particulate aerosol generator is an insufflator. According to some embodiments, the inhalation delivery device is a nebulizer, a metered-dose inhaler, or a dry powder inhaler (DPI). According to some embodiments, respirable particles range in size from about 1 to 10 microns, inclusive; or particles for nasal administration (insufflation), range in size from 10-500 μM, inclusive.

According to some embodiments, the dry powder may be produced by a spray drying process.

The compositions of the present invention may be in the form of suppositories for rectal administration of the composition. These compositions may be prepared by mixing the drug with a suitable nonirritating excipient such as cocoa butter and polyethylene glycols which are solid at ordinary temperatures but liquid at the rectal temperature and will therefore melt in the rectum and release the drug. When formulated as a suppository the compositions of the invention may be formulated with traditional binders and carriers, such as triglycerides.

In addition to the formulations described previously, the compositions of the described invention also can be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising any one or plurality of the active agents disclosed herein also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

### Additional Components

Additional active ingredients included in the compositions of the present disclosure to treat an epithelial-related condition include, without limitation, either alone or in combination, a serine protease inhibitor agent; a protective agent, an emollient, an astringent, an irritant, a keratolytic, a sun screening agent, a sun tanning agent, an antibiotic agent, a non-imidazole analog antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent.

Serine protease inhibitors: There are three distinct types of protein serine protease inhibitors. The largest group of protein inhibitors is canonical inhibitors (for example, Cucurbita maxima trypsin inhibitor (CMTI trypsin inhibitor). These are small proteins that block the enzyme at the distorted Michaelis complex reaction stage; the protease-binding loop of canonical inhibitors is kept in a well-ordered conformation [Krowarsch, D., et al. Cell Mol. Life Sci. (2003) 60: 2427-2444].

Serpins (serine protease inhibits) (e.g., alpha-1-antitrypsin, are much larger, typically 350-500 amino acids in size and are distributed from viruses to mammals [Id, citing Gettins, PG. Chem. Rev. (2002) 102: 4751-4804; Silverman, GA., et al. J. Biol. Chem. (2001) 276: 33293-33296]. Like canonical inhibitors, serpins interact with their target proteases in a substrate manner. Unlike canonical inhibitors, the binding loop of serpins is much longer, about 17 amino acids, and able to adopt different conformations. In contrast to canonical inhibitors, serpins utilize the kinetic features of a hydrolytic reaction to form a very stable acyl-enzyme intermediate. The enzyme-serpin complex is a covalent acyl-enzyme adduct and upon acylation, the protease is translocated by over 70 A from its initial recognition site [Id. citing Huntington, J A., et al. Nature (2000) 407: 923-926]. Serpins are the only family of serine protease inhibitors for which complex formation with non-serine enzymes (cysteine proteases (Id, citing Komiyama, T., et al. J. Biol. Chem. (1994) 269: 19331-19337) and aspartyl proteases (Id, citing Mathialagan, N. and Hansen, TR. Proc. Natl Acad. Sci. USA (1996) 93: 13653-13658) has been demonstrated. SerpinB1, also known as leukocyte elastase inhibitor (LEI) or monocyte/NE inhibitor (MNEI)) is another example [Majewski, P., et al. Front. Immunol. (2016) 7: 261].

Non-canonical inhibitors (e.g., ornithodorin: thrombin) interact through their N-terminal segment, which binds to the protease active site forming a short parallel β-sheet. These inhibitors, which only occur in blood sucking organisms and inhibit proteases involved in clot formation (thrombin or factor Xa) also form extensive secondary interactions with the target protease outside the active site. The classic example is recognition of thrombin by hirudin [Id, citing Stubbs, MT., et al. FEBS Letters (1995) 375: 103-107].

Mechanisms. Protein protease inhibitors can interact with proteases in different ways, although there are two mechanisms of interaction widely distributed in nature [Clemente, M. et al. Int. J. Mol. Sci. (2019) 20(6): 13453, citing Rawlings, N D et al. Biochem. J. (2004) 378: 705-716]. One of them is the irreversible trapping reaction; the best-characterized families of protease inhibitors that show this mechanism correspond to the families of serpins (I4), α2 macroglobulins (I39) and baculovirus protein p35 inhibitors (I50) [Id, citing Rawlings, N D et al., Biochem. J. (2004) 378: 705-16; Rawlings, N D. Biochimie (2010) 92: 1463-1483]. In this type of inhibition mechanism, the protease-inhibitor interaction induces the cleavage of an internal peptide bond in the inhibitor structure, triggering a conformational change. This reaction is not reversible, and the inhibitor never recovers its initial structure. For this reason, the inhibitors that participate in trapping reactions are also known as suicide inhibitors.

The other mechanism generally observed of protease-inhibitor interaction is known as a tight-binding reaction. This mechanism is also called a standard mechanism [Id, citing Lashowski, M. and Qasim, M A. Biochim. Biphys. Acta (2000) 1477: 324-337; Farady, C J and Craik, C S (ChemBioChem. (2010) 11: 2341-2346). All inhibitors that operate by this mechanism are canonical and it was demonstrated for serine protease inhibitors [Id, citing Rawlings, N D., et al. Biochem. J. (2004) 378: 705-716]. The majority of plant serine protease inhibitors (SPIs) adopt the standard mechanism of inhibition [Id, citing Bateman, KS and James, M N. Curr. Protein Pept. Sci. (2011) 12: 340-347]. In tight-binding reactions, the inhibitors interact with the protease active site (P1) in a similar way to the enzyme-substrate interaction. The protease-inhibitor complex co-exists in a stable equilibrium among the intact form of the inhibitor and the modified forms of the inhibitor where the peptide bond of the reactive site is cleaved. Therefore, the inhibitor in the complex is dissociated to its intact or its modified form. The canonical inhibitors can also inhibit serine proteinases with differing P1 specificities.

The Bowman Birk, Potato II and Kunitz families are able to target more than one proteinase at a time, often with different specificities [Id, citing Joshi, R S., et al. Biochim. Biophys. Acta (2013) 1830: 5087-5094]. Plant SPIs that apply this strategy allow plants to prepare themselves against unwanted proteolytic activity, whether to control development or to defend against pest attack.

Chemical serine protease inhibitors. Phenyl-methyl-sulfonyl fluoride (PMSF) is an exemplary chemical protease inhibitor that reacts with serine residues to inhibit trypsin, chymotrypsin, thrombin and papain.

Protectives: Protectives as described herein may take the form of dusting powders, adsorbents, mechanical protective agents, and plasters. Dusting powders are relatively inert and insoluble materials that are used to cover and protect epithelial surfaces, ulcers and wounds. Usually, these substances are finely subdivided powders that absorb moisture and can act as a desiccant. The absorption of skin moisture decreases friction and also discourages certain bacterial growth. Some of the materials used as protective adsorbents include bentonite, insoluble salts of bismuth, boric acid, calcium carbonate, (precipitated), cellulose, corn starch, magnesium stearate, talc, titanium dioxide, zinc oxide, and zinc stearate. Protectives can also be administered to the skin to form an adherent, continuous film that may be flexible or semi-rigid depending on the materials and the formulations as well as the manner in which they are applied. This material may serve several purposes including providing occlusion from the external environment, providing chemical support, and serving as vehicles for other medicaments. Mechanical protectives are generally either collodions or plasters. Examples include aluminum hydroxide gel, collodium, dimethicone, petrolatum gauze, absorbable gelatin film, absorbable gelatin sponge, zinc gelatin, kaolin, lanolin, anhydrous lanolin, mineral oil, mineral oil emulsion, mineral oil light, olive oil, peanut oil, petrolatum, silicones, hydrocolloids and the like.

According to some embodiments, protectives included in the composition of the invention are demulcents. According to some embodiments, the irritant is a rubefacient.

Sun screening agents: Representative examples of sun screening agents usable in context of the described invention include, without limitation, p-aminobenzoic acid and its salts and derivatives thereof (ethyl, isobutyl, glyceryl esters; p-dimethylaminobenzoic acid); anthranilates (i.e., o- amino-benzoates; methyl, menthyl, phenyl, benzyl, phenylethyl, linalyl, terpniyl, and cyclohexenyl esters); salicylates (amyl, phenyl, octyl, benzyl, menthyl, glyceryl, and di-propylene glycol esters); cinnamic acid derivatives (menthyl and benzyl esters, a-phenyl cinnamonitrile; butyl cinnaraoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzylacetone and benzylacetophenone; naphtholsulfonates (sodium salts of 2-naphthol-3,6-disulfonic and of 2-naphthol-6,8-disuIfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyqumoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one) and 4-isopropyl-di-benzoylmethane, and any combination thereof.

Sunless tanning agents. Representative examples of sunless tanning agents usable in the described invention include, without limitation, dihydroxyacetone, glyceraldehyde, indoles and their derivatives. The sunless tanning agents can be used in combination with the sunscreen agents.

Vitaminized peptides: According to some embodiments, the composition further comprises vitaminized peptides. In some embodiments, the composition further comprises vitaminized proteins.

Cleansing agents: Exemplary cleansing agents which may be use in the described invention include surfactant based cleansing agents, examples of which have been listed hereinabove. Other non-surfactant-based cleansing agents known to those of skill in the art may also be employed.

Anesthetic agents: In some embodiments, the composition further comprises anesthetic agents that result in a reduction or loss of sensation. Non-limiting examples of anesthetic agents that are suitable for use in the context of the described invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

Anti-acne agents: In some embodiments, the composition further comprises anti-acne agents that alleviate the symptoms of acne. Nonlimiting examples of anti-acne agents include, without limitation, keratolyses, such as salicylic acid, sulfur, glycolic, pyruvic acid, resorcinol, and N-acetylcysteine; and retinoids such as retinoic acid and its derivatives (e.g., cis and trans, esters).

Antibiotic agents: In some embodiments, the composition further comprises antibiotic agents that have the capacity to inhibit the growth of or destroy bacteria. Nonlimiting examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetatnet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

Anti-dandruff agents: In some embodiments, the composition further comprises anti-dandruff agents that reduce, eliminate, or prevent a scurf from forming on skin. Nonlimiting examples of anti-dandruff agents include zinc pyrithione, shale oil and derivatives thereof such as sulfonated shale oil, selenium sulfide, sulfur; salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazole nitrate and any possible stereo isomers and derivatives thereof such as anthralin, piroctone olamine (Octopirox), selenium sulfide, and ciclopiroxolamine, and mixtures thereof.

Anti-histamine agents: In some embodiments, the composition further comprises anti-histamine agents that counteract histamine in the body and are used for treating allergic reactions. Non-limiting examples of antihistamines usable in context of the described invention include chlorpheniramine, brompheniramine, dexchlorpheniramine, tripolidine, clemastine, diphenhydramine, promethazine, piperazines, piperidines, astemizole, loratadine and terfenadine.

Antioxidant agents: In some embodiments, the composition further comprises antioxidant agents that inhibit oxidation or reactions promoted by oxygen and peroxide. Non-limiting examples of antioxidants that are usable in the context of the described invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, ó-hydroxy-ZjSjTjδ-tetramethylchroman-1-carboxylic acid (commercially available under the tradename Trolox®), gallic acid and its alkyl esters, especially propyl gallate, uric acid and its salts and alkyl esters, sorbic acid and its salts, lipoic acid, amines (e.g., N5N-diethyl;hydroxylamine, amino-guanidine), sulfhydryl compounds (e.g., glutathione), dihydroxy fumaric acid and its salts, glycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Anti-protozoal agents: In some embodiments, the composition further comprises anti-protozoal agents that have the capacity to inhibit the growth of or destroy protozoans. Nonlimiting examples of anti-protozoal agents include pyrimethamine (Daraprim®), sulfadiazine, and Leucovorin.

Antipruritic agents: In some embodiments, the composition further comprises antipruritic agents that reduce, eliminate, or prevent itching. Nonlimiting examples of antipruritic agents include pharmaceutically acceptable salts of methdilazine and trimeprazine.

Anti-wrinkle agents: In some embodiments, the composition further comprises anti-wrinkle agents that are intended to reduce the appearance of wrinkles in the skin. Nonlimiting examples of anti-wrinkle agents include retinols, Bakuchiol, vitamin C (ascorbic acid), niacinamide; tranexamic acid; azelaic acid, salicylic acid and derivatives thereof, sulfur-containing D and L amino acids and their derivatives and salts, particularly the N-acetyl derivatives, an example of which is N-acetyl L-cysteine; thiols, e.g. ethane thiol; alpha-hydroxy acids, e.g. glycolic acid, and lactic acid; phytic acid, lipoic acid; lysophosphatidic acid, and skin peel agents (e.g., phenol and the like).

Emollients: In some embodiments, the composition further comprises emollients that soften the skin by forming an occlusive oil film on the stratum corneum, preventing drying from evaporation in the deeper layers of skin. Nonlimiting examples of emollients include butters, such as Aloe Butter, Almond Butter, Avocado Butter, Cocoa Butter, Coffee Butter, Hemp Seed Butter, Kokum Butter, Mango Butter, Mowrah Butter, Olive Butter, Sal Butter, Shea Butter, glycerin, and oils, such as Almond Oil, Aloe Vera Oil, Apricot Kernel Oil, Avocado Oil, Babassu Oil, Black Cumin Seed Oil, Borage Seed Oil, Brazil Nut Oil, Camellia Oil, Castor Oil, Coconut Oil, Emu Oil, Evening Primrose Seed Oil, Flaxseed Oil, Grape Seed Oil, Hazelnut Oil, Hemp Seed Oil, Jojoba Oil, Kukui Nut Oil, Macadamia Nut Oil, Meadowfoam Seed Oil, Mineral Oil, Neem Seed Oil, Olive Oil, Palm Oil, Palm Kernel Oil, Peach Kernel Oil, Peanut Oil, Plum Kernel Oil, Pomegranate Seed Oil, Poppy Seed Oil, Pumpkin Seed Oil, Rice Bran Oil, Rosehip Seed Oil, Safflower Oil, Sea Buckthorn Oil, Sesame Seed Oil, Shea Nut Oil, Soybean Oil, Sunflower Oil, Tamanu Oil, Turkey Red Oil, Walnut Oil, Wheatgerm Oil.

Hypopigmenting agents: In some embodiments, the compositions further comprise hypopigmenting agents that are capable of depigmenting the skin. Nonlimiting examples of hypopigmenting agents include hydroquinones, mequinol, and various protease inhibitors including serine protease inhibitors, active soy and retinoic acid.

Nonsteroidal anti-inflammatory agents: In some embodiments, the compositions further comprise nonsteroidal anti-inflammatory agents that are aspirin-like in their action. Nonlimiting examples of nonsteroidal anti-inflammatory agents include ibuprofen (Advil®), naproxen sodium (Aleve®), and acetaminophen (Tylenol®). Additional examples of non-steroidal anti-inflammatory agents that are usable in the context of the described invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopmac, zidometacin, acematacin, fentiazac, zomepirac, clindanac, oxepinac, felbinac, and ketorolac; fenamates, such as mefenamic, meclofenamic, flufenamic, nifiumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alrninoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is particularly useful for topical application.

The topical compositions of the described invention can be applied locally to the skin and may be in any form including solutions, oils, creams, ointments, gels, lotions, shampoos, milks, cleansers, moisturizers, sprays, skin patches and the like.

Additional compositions of the described invention can be readily prepared using technology which is known in the art such as described in Remington's Pharmaceutical Sciences, 18th or 19th editions, published by the Mack Publishing Company of Easton, Pennsylvania.

According to the foregoing embodiments, the composition may be administered once, for a limited period of time, or as a maintenance therapy over an extended period of time, for example until the condition is ameliorated, cured, or for the life of the subject. A limited period of time may be for 1 week, 2 weeks, 3 weeks, 4 weeks and up to one year, including any period of time between such values, including endpoints. According to some embodiments, the composition may be administered for about 1 day, for about 3 days, for about 1 week, for about 10 days, for about 2 weeks, for about 18 days, for about 3 weeks, or for any range between any of these values, including endpoints. According to some embodiments, the composition may be administered for more than one year, for about 2 years, for about 3 years, for about 4 years, or longer.

According to the foregoing embodiments, the composition may be administered once daily, twice daily, three times daily, four times daily, or more.

### Methods of the Present Disclosure

The present disclosure provides methods comprising: (i) obtaining a biological sample comprising a population of extracellular vesicles (EVs) comprising a cargo composition; (ii) exposing the biological sample comprising the population of EVs comprising the cargo composition to one or more stress conditions to form a population of EVs comprising a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p; and
(ii) a signature of one or more proteins selected from the group consisting of BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82 and WNT5A.

### Methods of Obtaining a Biological Sample

The present disclosure provides methods for obtaining a biological sample. In some embodiments, obtaining a biological sample comprises obtaining a population of cells maintained in culture. In some embodiments, the population of cells maintained in culture is a heterogenous population of cells. In some embodiments, the population of cells maintained in culture is a homogenous population of cells. In some embodiments, the population of cells maintained in culture is derived from an immortalized cell line (i.e., cells that have been modified to proliferate indefinitely). In some embodiments, the population of cells maintained in culture is derived from a primary cell line (i.e., cells that are isolated directly from living tissue).

In some embodiments, obtaining a population of cells maintained in culture comprises obtaining a population of cells, wherein the cells are cultured in a two-dimensional culture system. In some embodiments, the two-dimensional culture involves growing cells in a single monolayer on flat surfaces surface-treated to promote adherence of the cells to the flat surface (Zieger, A S et al. Acta Biomaterialia (2013) 9 (7): 7354-7361). In some embodiments, the flat surfaces include, without limitation, petri dishes, cell culture dishes, multi-well plates, and cell culture flasks.

In some embodiments, obtaining a population of cells maintained in culture comprises obtaining a population of cells, wherein the cells are cultured in a three-dimensional culture system. In some embodiments, the three-dimensional culture system involves allowing cells to grow in three dimensions, mimicking in vivo tissue architecture. In some embodiments, the three-dimensional culture includes, but is not limited to spheroids (i.e., spherical clusters of cells, often grown in low-adhesion plates or via hanging drop methods), organoids (i.e., 3D structures derived from stem cells that self-organize to emulate the functional characteristics of an organ), and matrix-supported cultures (i.e., cells suspended within a 3D gel matrix. The term “matrix” as used herein refers to a three dimensional network of fibers that contains voids (or “pores”) where the woven fibers intersect. The structural parameters of the pores, including the pore size, porosity, pore interconnectivity/tortuosity and surface area, can affect how substances (e.g., fluid, solutes) move in and out of the matrix.

In some embodiments, the matrix-supported culture comprises a Matrigel® matrix (i.e., a complex matrix derived from the secretion of mouse Engelbreth-Holm-Swarm tumors). Matrigel® is poorly defined, and, because it originates from mouse cells, potentially immunogenic when used to culture human cells [Kozlowski, M T et al. Commun. Biol. (2021) 4 (1): 1387 doi: 10.1038/s42003-021-02910-8]. In some embodiments, the matrix-supported culture comprises a hydrogel matrix (i.e., a natural or synthetic crosslinked porous polymer network that possesses high water content and mimics the extracellular matrix, [Tibbitt, M W and Anseth, K S. Biotechnol. Bioeng. (2009) 103(4): 655-663) citing Lee, K Y and Mooney, D J. Chem. Rev. (2001) 101(7): 1869-1880]. In some embodiments, the matrix-supported culture comprises a collagen matrix (a structured, biocompatible scaffold derived from collagen (often bovine or porcine) used in medicine to support soft tissue regeneration, wound healing, and bone grafting that acts as a template for new tissue growth, providing structural stability and facilitating cell adhesion, while gradually degrading as the body heals, see Dong, C. and Lv, Y. Polymers (Basel) (2016) 8 (2): 42). In some embodiments, the matrix-supported culture comprises a laminin matrix (see, e.g., Garg, K. Advances in Tissue Engineering & Regenerative Med. (2017) 2 (3): 00033). In some embodiments, the matrix-supported culture comprises a fibronectin matrix (see, e.g., Jordahl, S. et al. Adv. Mater. (2019) 31 (46): e1904580).

In some embodiments, obtaining a population of cells maintained in culture comprises obtaining a population of cells, wherein the cells are maintained in a large-scale culture system, wherein the large-scale culture system comprises a scalable bioreactor culture system allowing efficient scale up to greater cell quantities in well-controlled environments. In some embodiments, the bioreactor culture system includes, but is not limited to stirred tank bioreactors (i.e., a bioreactor that uses mechanical impellers for mixing cultured cells), air-lift bioreactors (i.e., a bioreactor that uses air bubbles for mixing and oxygen transfer without mechanical agitators), fixed bed bioreactors (i.e., a bioreactor that contains a stationary bed of particles or solid matrix where cells grow), fluidized bed bioreactors (i.e., a bioreactor similar to a fixed bed bioreactor, wherein the particles are suspended by upward fluid flow), or hollow fiber bioreactors (i.e., a bioreactor that mimics capillary networks to grow cells using semi-permeable, hollow fiber, membranes within a cartridge).

In some embodiments, obtaining a biological sample comprises obtaining a tissue sample. The term “tissue” as used herein means a group of cells having a common function or structure. In some embodiments, the tissue sample is obtained from a subject. In some embodiments, the subject is a normal healthy subject. In some embodiments, the subject is a subject having or suspected of having a disease. According to some embodiments, the subject is a mammal. According to some embodiments, the subject is a non-human mammal or a human subject.

In some embodiments, the tissue sample is obtained from the subject by a surgical resection or excision. In some embodiments, a surgical resection removes part or all of a diseased or damaged organ, tissue, or tumor from the body. In some embodiments, the removed organ, tissue, or tumor is utilized for further analysis.

In some embodiments, the tissue sample is obtained from the subject by a liquid biopsy (i.e., a laboratory test done on a sample of a bodily fluid (e.g., blood, plasma, urine, cerebrospinal fluid, saliva, etc.)). In some embodiments, the tissue sample is obtained from the subject using a tissue biopsy. Types of tissue biopsies include, without limitation, a bone marrow biopsy, a core biopsy, an excisional or incisional biopsy, a needle biopsy, a punch biopsy, a shave biopsy, and an endoscopic biopsy.

In some embodiments, the tissue sample is obtained from the subject by a bone marrow biopsy (i.e., a medical procedure that removes a small sample of bone marrow tissue using a specialized needle and syringe to examine it for diseases).

In some embodiments, the tissue sample is obtained from the subject by a needle biopsy (i.e., a medical procedure that uses a hollow needle to remove small samples of fluids, cells, or tissues).

In some embodiments, the tissue sample is obtained from the subject by a core biopsy, a medical procedure in which a hollow needle is used to draw a column of tissue out of a suspicious area to check for diseased cells (core biopsy or core needle biopsy or CNB, for example, obtaining a tissue sample from the cervix).

In some embodiments, the tissue sample is obtained from the subject by a surgical biopsy procedure if the cells in question cannot be accessed with other biopsy procedures. Examples of surgical biopsy procedures include excisional biopsy where an entire lump or area of tissue is removed (e.g., surgery to remove breast tissue; surgery to remove a lymph node) or incisional biopsy (e.g., a scalpel is used to remove a small area of skin).

Skin biopsy procedures include, without limitation, punch biopsy, shave biopsy, incisional biopsy and excisional biopsy. In some embodiments, the tissue sample is obtained from the subject using a punch biopsy (i.e., a small circular hollow tool used to remove a small, fill-thickness core of tissue). In some embodiments, the tissue sample is obtained from the subject using a shave biopsy (i.e., obtaining a tissue or cell sample through the use of a razor blade or scalpel to scrape up at thin sample of the top layers of the skin).

In some embodiments, the tissue sample is obtained from the subject by an endoscopic biopsy (i.e., obtaining a biological sample using an endoscope (meaning a lighted flexible tube with a camera at the end that is inserted into a bodily opening during endoscopic procedures while the patient is sedated). Examples of endoscopic biopsy procedures include cystoscopy to collect tissue inside the bladder; bronchoscopy to collect tissue from inside a lung; and colonoscopy to collect tissue from inside the colon.

In some embodiments, obtaining the biological sample comprises obtaining a tissue sample from a subject and maintaining the tissue ex vivo. In some embodiments, obtaining a tissue sample from a subject and maintaining the tissue sample ex vivo further comprises processing the tissue for ex vivo tissue preservation. In some embodiments, processing the tissue sample for ex vivo tissue preservation comprises: (i) immediately after obtaining the tissue sample, cooling the tissue at 4 °C (or on ice) in sterile culture medium; (ii) cleaning the tissue sample in antibiotic containing medium to remove contaminants; and (iii) thinning the tissue sample into small fragments (1-3 mm³) to allow nutrients to diffuse into the center of the tissue fragment.

In some embodiments, the tissue sample is obtained from an epithelial tissue (i.e., tissue that covers exterior surfaces, lines internal cavities, and forms glands). Nonlimiting examples of epithelial tissue include simple squamous epithelium (i.e., a type of tissue composed of thin, flat, scale-like cells that form protective barriers or facilitate diffusion in the body), stratified squamous epithelium (i.e., a multi-layered tissue composed of flattened, scale-like cells at the surface and cuboidal/columnar cells at the base), simple cuboidal epithelium (i.e., a single layer of cube-shaped cells, roughly as wide as they are tall), stratified cuboidal epithelium (i.e., a type composed of two or more layers of cube-shaped cells), simple columnar epithelium (i.e., a single layer of tall, narrow epithelial cells specialized from absorption, secretion of mucus/enzymes, and protection), pseudostratified columnar epithelium (i.e., a specialized, single-layered tissue that appears stratified), and transitional epithelium (i.e., a specialized, multi-layered tissue that lines the urinary tract). In some embodiments, the tissue sample is obtained from connective tissue (i.e., tissues that bind, support, protect, and integrate, or separate other tissues). Nonlimiting examples of connective tissue include loose connective tissue (i.e., a flexible, gel-like, vascularized tissue that cushions organs, binds skin, and holds tissue together), dense connective tissue (i.e., a strong, fibrous tissue high in collagen fibers that provides structural support, protection, and flexibility to the body), adipose tissue (i.e., a specialized connective tissue composed mainly of fat-storing cells), bone (i.e., a specialized, mineralized connective tissue that forms the structural framework of the skeleton), cartilage (i.e., a tough, flexible, and resilient connective tissue that cushions joints, reduces friction between bones, and provides structural support), and blood (i.e., a specialized, liquid connective tissue that circulates through the body's vessels). In some embodiments, the tissue sample is obtained from muscle tissue (i.e., tissue that is composed of elongated, contractile cells that generate force through contraction, enabling movement). Nonlimiting examples of muscle tissue includes skeletal muscle (i.e., a voluntary, striated muscle type attached to bones by tendons, forming bundles of muscle fibers that control body movement, posture, and heat generation), smooth muscle (i.e., an involuntary, non-striated muscle found in the walls of hollow organs, blood vessels, and airways, designed to manage passage width and transport substances), and cardiac muscle (i.e., a specialized, involuntary, striated muscle tissue found exclusively in the heart walls).

In some embodiments, obtaining a biological sample comprises obtaining a sample of a plant tissue Plant tissues fall into two general categories: meristematic tissue, and permanent (or non-meristematic) tissue. Meristematic tissue is functionally equivalent to stem cells in animals: undifferentiated cells that continue to divide and generate new cells and new tissues. However, animal stem cells contribute to replacing aging or injured tissues, while plant meristems contribute to new growth over the life of the plant. In plants, permanent tissue consists of plant cells that are no longer actively dividing. Meristems produce cells that quickly differentiate, or specialize, and become cells of permanent tissue; they differentiate into three main tissue types: dermal (meaning protective plant tissue covering the outermost part of the plant that controls gas exchange), vascular (meaning tissue made up of xylem and phloem that transports food and water throughout the plant), and ground tissue (meaning plant tissue involved in photosynthesis that provides support, and stores water and sugars). Each plant organ (roots, stems, leaves) contains all three tissue types [openstax.org/books/biology/pages/30-1-the-plant-body].

In some embodiments, the plant tissue sample comprises a whole plant or a portion of a plant. In some embodiments, a plant sample includes, but is not limited to roots, stems, leaves, flowers (the primary reproductive structures), seeds (the embryonic stage of the plant life cycle), fruits (meaning the mature and ripened ovaries of flowers), liquid extracts of any of the foregoing, or a combination thereof. In some embodiments, the plant sample is obtained by harvesting, cutting, excising, or otherwise removing plant material from a plant.

### Methods of Stress Exposure

The present disclosure provides methods for exposing a biological sample to one or more stress conditions that modulate the cargo of the population of EVs contained in the biological sample. In some embodiments, the one or more stress conditions is sufficient to modulate gene expression, signaling pathways, metabolism, cellular responses, or a combination thereof of the biological sample comprising the EVs. In some embodiments, exposure to the one or more stress conditions produces a population of EVs comprising a “tuned” cargo. In some embodiments, the one or more stress conditions comprises hypoxic stress, metabolic stress, mechanical stress, osmotic stress, chemical stress, heat stress, or a combination thereof.

In some embodiments, exposing the biological sample to hypoxic stress comprises reducing oxygen availability to the biological sample. In some embodiments, reducing the oxygen availability to the biological sample comprises placing the biological sample in a reduced oxygen environment, for example, a controlled atmosphere chamber or an incubator configured to maintain reduced oxygen levels. In some embodiments, the reduced oxygen environment is generated by replacing at least a portion of the ambient temperature with one or more gases. In some embodiments, the one or more gases comprise nitrogen, carbon dioxide, or a combination thereof.

In some embodiments, standard oxygen levels in the controlled oxygen environment or incubator comprise 18-21%. In some embodiments, the reduced oxygen levels of the controlled oxygen environment or incubator comprise from about 1% to about 5% oxygen concentration, inclusive, i.e., about 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% oxygen concentration.

In some embodiments, the biological sample is exposed to the reduced oxygen environment of from about 1% to 5% oxygen, inclusive for about 0.5 hour to about 72 hours, inclusive, i.e., about 0.5 hour, 0.6 hour, 0.7 hour, 0.8 hour, 0.9 hour, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, or 72 hours.

In some embodiments, hypoxic stress is measured by assessing the expression of one or more hypoxia-related genes. In some embodiments, hypoxia related genes include, but are not limited to, hypoxia-inducible factor 1 alpha (HIF-1α), hypoxia-inducible factor 2 alpha (HIF-2α), vascular endothelial growth factor (VEGF), carbonic anhydrous IX (CA IX), or glucose transporter 1 (GLUT1) (Wu D, Yotnda P. J Vis Exp. 2011 Aug. 12; (54):2899, Bao B, et al. PLoS One. 2012; 7(11):e50860, Mamun A A, et al. J Physiol Sci. 2020 Sep. 22; 70(1):44).

In some embodiments, exposing the biological sample to metabolic stress comprises altering the availability or utilization of nutrients in the biological sample. In some embodiments, altering the availability or utilization of nutrients in the biological sample comprises one or more of glucose deprivation, amino acid deprivation, lipid deprivation, protein deprivation, or a combination thereof.

In some embodiments, the biological sample is exposed to glucose deprivation, amino acid deprivation, lipid deprivation, protein deprivation, or a combination thereof for about 1 hour to about 72 hours, inclusive, i.e., about 0.5 hour, 0.6 hour, 0.7 hour, 0.8 hour, 0.9 hour, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, or 72 hours.

In some embodiments, metabolic stress is measured by using metabolic assays that measure ATP levels and determine cellular energy status, such as those disclosed in Zhang J, et al. Nat Protoc. 2012 May 10; 7(6):1068-85.

In some embodiments, exposing the biological sample to osmotic stress comprises a change in osmolarity relative to physiological conditions. In some embodiments, a change in osmolarity relative to physical conditions comprises hyperosmotic (i.e., increased osmolarity) conditions or hypoosmotic (i.e., decreased osmolarity) conditions.

In some embodiments, osmotic stress is induced by increasing the concentration of one or more osmotically active agents (i.e., solutes that create a higher concentration on one side of the membrane, drawing water to balance that concentration) in a medium contacting the biological sample. In some embodiments, osmotic stress is induced by decreasing the concentration of one or more osmotically active agents in a medium contacting the biological sample. In some embodiments, one or more osmotically active agents comprises salts and sugars, for example, sodium chloride, potassium chloride, fructose, glucose, lactose, or a combination thereof.

In some embodiments, the biological sample is exposed to a hyperosmotic or hypoosmotic solution for a period of time between 10 minutes to about 6 hours, inclusive, i.e., about 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, or 6 hours.

In some embodiments, osmotic stress is measured by analyzing changes in cell volume (i.e., under hyperosmotic conditions, cells shrink, under hypoosmotic conditions, cells swell), or using atomic force microscopy (AFM) (i.e., uses a tiny probe with a very sharp tip to sense small variations in force. When sufficiently close to the sample, the tip is affected by forces at the sample surface. This causes the tip to move, which in turn causes a cantilever to bend. The cantilever bending, or deflection, is detected optically using a laser and photodetector (a device that converts light into an electrical signal). It is used to detect changes in cellular stiffness resulting from osmotic pressure. Cellular stiffness has a positive correlation with osmotic pressure, meaning as the external osmotic pressure increases (hyperosmolarity), cell stiffness increases, and as it decreases (hypoosmolarity), cell stiffness decreases. AFM provides this information by directly measuring the mechanical response of cells in real-time under varying osmotic environments, utilizing both single-point measurements and force mapping to map local stiffness (Liao H S, et al. J Biomech Eng. 2018 May 1; 140(5):054502).

In some embodiments, exposing the biological sample to mechanical stress comprises exposing the biological sample to one or more physical forces. In some embodiments, the one or more physical forces comprises shear stress (meaning force per unit area acting parallel to a surface), stretching (meaning applying tension, an external pulling force), compression (a pushing force that compacts a material), agitation (meaning the process of inducing motion in a fluid (liquid or gas) through mechanical means), vibration (meaning forced oscillation of a system by an external periodic force), pressure changes, or a combination thereof.

In some embodiments, the biological sample is exposed to shear stress, stretching, compression, agitation, vibration, pressure changes, or a combination thereof for about 10 minutes to about 1 month, inclusive, i.e., 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, 6 hours, 7 hours, 8 hours, 9 hours, 10 hours, 11 hours, 12 hours, 14 hours, 16 hours, 18 hours, 20 hours, 22 hours, 1 day, 2 days, 3 days, 4 days, 5 days, 6 days, 7 days, 1 week, 2 weeks, 3 weeks, or 1 month.

The relative stiffness or deformability of a tissue type has considerable importance on how the cells interpret their surrounding matrix. Cell stiffness is generally expressed as the Young's modulus in the unit Pascals; adipose tissue is relatively soft, with an approximate Young's modulus of 1 kPa (Elkin, B S et al. J. Neurotrauma (2007) 24 (5): 812-822] compared to muscles, tendon and bone tissue, which are much stiffer, measuring from 6 MPa-40 GPa (Discher, D E et al. Science (2005) 310: 1139; Reilly, D T and Burstein, AH, J. Bone and Joint Surgery (1974) 56: 1001-1022). The composition and structure of the cell membrane and cytoskeleton are major determinants of deformability, and studies have identified signaling pathways that affect cytoskeletal dynamics both directly and by altered gene expression [Galie, P A, et al. Biochem. J. (2022) 479 (17): 1825-1842]. In some embodiments, mechanical stress is measured by analyzing changes in cell morphology, using atomic force microscopy (AFM) (i.e., used to detect changes in cellular stiffness resulting from mechanical stress, see, Liao H S, et al. 2018 May 1; 140(5):054502), using traction force microscopy (i.e., measures the force a cell exerts on its substrate by tracking the movement of embedded fluorescent beads, calculating mechanical stress, see Zancla A, et al. J Biol Chem. 2022 May; 298(5):101867).

In some embodiments, exposing the biological sample to chemical stress comprises exposing the biological sample to one or more exogenous chemical agents. In some embodiments, the one or more chemical agents comprises cytokines (for example, tumor necrosis factor-alpha (TNF-α), interleukin-1beta (IL-1), interleukin 6 (IL-6), interferon-gamma (IFN-γ)), reactive oxygen species (for example, superoxide anion radical, hydrogen peroxide, hydroxyl radical, nitric oxide), toxins (for example, arsenic, cadmium, formaldehyde), pH altering agents (hydrochloric acid, sorbic acid, acetic acid, ammonium chloride, sodium hydroxide), or a combination thereof.

In some embodiments, the biological sample is exposed to cytokines, reactive oxygen species, toxins, pH altering agents, or a combination thereof for a time period between 1 hour to about 72 hours, inclusive, i.e., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, or 72 hours.

In some embodiments, chemical stress is measured by assessing oxidative stress and cellular damage (i.e., by measuring the amount of ROS, double strand breaks in DNA, see, Katerji M, et al. 2019 Mar. 12; 2019:1279250, Halliwell B, et al. 2004 May; 142(2):231-55) lipid damage by measuring lipid peroxidation (see, Ayala A, et al. Oxid Med Cell Longev. 2014; 2014:360438, Kodali S T, et al. 2020 Aug. 9. In: Berliner L J, Parinandi N L, editors. Measuring Oxidants and Oxidative Stress in Biological Systems [Internet]. Cham (CH): Springer; 2020. Chapter 5. Available from: .ncbi.nlm.nih.gov/books/NBK566435) and protein damage by measuring protein modifications (e.g., oxidation, protein carbonyl content, see Kehm R, et al. Redox Biol. 2021 June; 42:101901, Raftery M J. 2014 July; 19(4):140-7).

In some embodiments, exposing the biological sample to heat stress comprises exposing the biological sample to excessive temperatures (i.e., heat shocking the biological sample). In some embodiments, exposing the biological sample to excessive temperatures includes exposing the biological sample to temperatures of about 30 °C to about 50 °C, inclusive, i.e., about 30 °C, 31 °C, 32 °C, 33 °C, 34 °C, 35 °C, 36 °C, 37 °C, 38 °C, 39 °C, 40 °C, 41 °C, 42 °C, 43 °C, 44 °C, 45 °C, 46 °C, 47 °C, 48 °C, 49 °C, or 50 °C.

In some embodiments, the biological sample is exposed to excessive temperatures for a period of time between 10 minutes to about 6 hours, inclusive, i.e., about 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, or 6 hours.

In some embodiments, the biological sample is exposed to excessive temperatures in a controlled environment for example, a chamber, a water bath, an incubator, or a bioreactor. In some embodiments, heat stress is measured by assessing the expression of heat shock proteins, including, but not limited to one or more of HSP40, HSP60, HSP70, and HSP90.

### Methods of Extracellular Vesicle Isolation and Purification

The present disclosure provides methods for isolating and purifying a population of EVs from the biological sample. In some embodiments, the population of EVs is isolated and purified from a biological sample that has been exposed to one or more stress conditions.

In some embodiments, the population of EVs is isolated from the biological sample by a laboratory technique. Examples of laboratory techniques include differential centrifugation, density gradient centrifugation, size exclusion chromatography, tangential flow filtration, precipitation, affinity capture, acoustic separation, etc. In some embodiments, the population of EVs is isolated from the biological sample by differential centrifugation (see, Momen-Heravi F, et al. Biol Chem. 2013 October; 394(10):1253-62). In some embodiments, the population of EVs is isolated from the biological sample by density gradient centrifugation (see, Momen-Heravi F, et al. Biol Chem. 2013 October; 394(10):1253-62). In some embodiments, the population of EVs is isolated from the biological sample by size exclusion chromatography (see, Momen-Heravi F, et al. Biol Chem. 2013 October; 394(10):1253-62). In some embodiments, the population of EVs is isolated from the biological sample by tangential flow filtration (TFF) (see, Kim K, et al. APL Bioeng. 2021 Feb. 10; 5(1):016103, Visan K S, et al. J Extracell Vesicles. 2022 September; 11(9):e12266). In some embodiments, the EVs are isolated from the biological sample by precipitation. In some such embodiments, EV precipitation involves adding polymers (e.g., polyethylene glycol (PEG)) to the biological sample, which alters the solubility of the sample to force EVs out of solution, followed by centrifugation (see, Zhang X, et al. J Extracell Vesicles. 2020 Jul. 14; 9(1):1791450). In some embodiments, the population of EVs is isolated from the biological sample using affinity capture (see, Lo T W, et al. Lab Chip. 2020 May 19; 20(10):1762-1770). In some embodiments, the population of EVs is isolated from the biological sample using acoustic separation (see, Wu M, et al. Lab Chip. 2019 Mar. 27; 19(7):1174-1182. Erratum in: Lab Chip. 2020 Sep. 21; 20(18):3472).

In some embodiments, the population of EVs is isolated from the biological sample using microfluidic isolation (see, Meggiolaro, A. et al. Biosensors (Basel). 2022 Dec. 29; 13(1):50). Nonlimiting examples of microfluidic isolation strategies include filtration systems (for example, using nanofiltration to separate EVs from biological samples by passing fluids through channels, typically removing cell debris first, followed by trapping EVs on porous membranes, see, Meggiolaro, A. et al. Biosensors (Basel). 2022 Dec. 29; 13(1):50), immunoaffinity capture (for example, using microchannels that are coated with antibodies targeting specific surface proteins to trap specific EV subsets, see, Meggiolaro, A. et al. Biosensors (Basel). 2022 Dec. 29; 13(1):50), size-exclusion chromatography on chip (for example, using microfluidic channels filled with a porous matrix to separate particles whereby smaller particles enter the pores and larger particles that cannot enter the pores pass through the matrix faster, see, Meggiolaro, A. et al. Biosensors (Basel). 2022 Dec. 29; 13(1):50), inertial/spiral microfluidics (for example, manipulating the inertial force to separate particles of different sizes, directing smaller EVs laterally within a channel, see, Meggiolaro, A. et al. Biosensors (Basel). 2022 Dec. 29; 13(1):50), and dielectrophoresis (for example, using an alternating current to create electric fields on a chip, and concentrating EVs based on their dielectric properties while washing away other components, see, Meggiolaro, A. et al. Biosensors (Basel). 2022 Dec. 29; 13(1):50).

In some embodiments, the methods comprise characterizing the isolated and purified population of EVs by purity, size, quantity, surface marker composition, or a combination thereof using nanoparticle tracking analysis (NTA) (see, Kowkabany G, et al. Molecules. 2024 Oct. 1; 29(19):4672). Nonlimiting examples of NTA include standard light-scattering NTA (which uses a laser to illuminate nanoparticles in a liquid suspension, whereby high-contrast images are captured and the Brownian motion of each particle is tracked to determine its hydrodynamic diameter, see, Kowkabany G, et al. Molecules. 2024 Oct. 1; 29(19):4672), fluorescent NTA (which employs laser sources with optical filters to specifically identify fluorescently labeled particles, which is crucial for distinguishing specific sub-populations, such as biomarker-positive EVs, from background particles, see, Kowkabany G, et al. Molecules. 2024 Oct. 1; 29(19):4672), interferometric NTA (a next-generation technology based on interferometric scattering microscopy (iSCAT), which enhances the signal of weak scatterers, allowing for improved sizing and detection of smaller particles compared to traditional NTA, see Kashkanova, A. D., et al. Journal of Extracellular Vesicles, 12, e12346), and multi-spectral NTA, which uses multi-spectral illumination with multiple laser wavelengths (e.g., 450 nm, 520 nm, 635 nm) to characterize a broader range of particle sizes simultaneously (see, particle-metrix.com/blogs/application-notes/colocalization-nta-of-multi-stained-platelet-derived-msc-ev-preparations).

In some embodiments, the methods comprise characterizing the isolated and purified population of EVs by purity, size, quantity, surface marker composition, or a combination thereof using resistive pulse sensing (RPS) (see, Song, Y. et al. Micromachines (Basel) (2017) 8 (7): 204). In some embodiments, methods comprise characterizing the isolated and purified population of EVs by purity, size, quantity, surface marker composition, or a combination thereof using vesicle flow cytometry (see, Welsh, J A et al. Front. Cell Dev. Biol. (2017) 5: 78). In some embodiments, methods comprise characterizing the isolated and purified population of EVs by purity, size, quantity, surface marker composition, or a combination thereof using a microfluidic device 9 (see, Meggiolaro, A. et al. Biosensors (Basel). 2022 Dec. 29; 13(1):50). In some embodiments, methods comprise characterizing the isolated and purified population of EVs by purity, size, quantity, surface marker composition, or a combination thereof using super-resolution nanoimaging (see, Bagci C, et al. Neurophotonics. 2022 April; 9(2):021903). Nonlimiting examples of super-resolution nanoimaging include single-molecule localization microscopy (a technique that maps individual, blinking molecules over thousands of frames to construct a high resolution image, see Lennon K M, et al. J Extracell Vesicles. 2019 Nov. 4; 8(1):1685634), stimulated emission depletion (a technique that scans samples with a donut shaped laser to deplete surrounding fluorescence, narrowing the excitation spot to nanometer scales, see, Bagci C, et al. Neurophotonics. 2022 April; 9(2):021903), structured illumination microscopy (a technique that uses patterned light to generate patterns and computationally restructures images with double resolution, see Alexandre, L. et al. ACS Nano 2025 19 (27), 24148-24173).

In some embodiments, methods comprise characterizing the isolated and purified population of EVs by purity, size, quantity, surface marker composition, or a combination thereof using transmission electron microscopy (TEM) (see, Corona M L, et al. Methods Mol Biol. 2023; 2668:33-43). Nonlimiting examples of transmission electron microscopy include conventional TEM (whereby a wide, static beam illuminates the entire sample to form an image for high-resolution of internal structures, see, Corona M L, et al. Methods Mol Biol. 2023; 2668:33-43), scanning TEM (whereby the electron beam is focused into a narrow spot and the beam is scanned across the sample in a raster pattern, similar to a scanning electron microscope, which allows for better image contrast and simultaneous acquisition of analytical data. see, Corona M L, et al. Methods Mol Biol. 2023; 2668:33-43), cryo-TEM (whereby specimens are kept in a frozen, vitrified state (typically using liquid nitrogen) to observe biological molecules and tissues in their native state without dehydration, see, Cizmar P, et al. Methods Mol Biol. 2017; 1660:221-232), and environmental TEM (which allows for the observation of samples under specific gas and temperature environments, see, Corona M L, et al. Methods Mol Biol. 2023; 2668:33-43).

### Methods for Improving Appearance of Human Skin

In some embodiments, the methods of the present disclosure further comprise a method for improving appearance of human skin, comprising: (i) obtaining a biological sample comprising a population of extracellular vesicles (EVs) comprising a cargo composition; (ii) exposing the biological sample comprising the population of EVs comprising the cargo composition to one or more stress conditions to form a population of EVs comprising a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo, and (v) applying the composition topically to human skin, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p; and
(ii) a signature of one or more proteins selected from the group consisting of BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82 and WNT5A.

In some embodiments, the methods described herein comprise administering topically to skin of a subject the composition comprising a purified population of EVs comprising a “tuned” cargo. In some embodiments, the composition is administered to facial skin. In some embodiments, the composition is administered to one or more regions of facial skin, including, the periorbital region, the cheek, the forehead, the chin, or a combination thereof.

In some embodiments, the method improves one or more parameters of skin appearance, compared to a control. In some embodiments, the control is a subject that was not administered topically to skin the composition comprising a purified population of EVs comprising a “tuned” cargo. In some embodiments, the one or more parameters of skin appearance comprise wrinkle reduction, increased skin firmness, increased skin elasticity, increased skin luminosity, decreased skin roughness, or a combination thereof.

In some embodiments, the method reduces the appearance of wrinkles. In some embodiments, the wrinkles comprise crow's feet (i.e., small lines radiating from the outer corners of the eyes), frown lines (i.e., vertical lines between the eyebrows), forehead lines (i.e., horizonal lines across the forehead), laugh lines (i.e., deep creases running from the nose side to the mouth corners), marionette lines (i.e., vertical liens running from the corners of the mouth down to the chin), lip lines (i.e., small vertical lines around the mouth), bunny lines (i.e., horizonal wrinkles across the bridge of the nose), or a combination thereof.

In some embodiments the method reduces the appearance of wrinkles by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to a control.

In some embodiments, the method reduces skin roughness. In some embodiments, skin roughness comprises an uneven, textured skin surface. In some embodiments, skin roughness manifests as flaking, scaling, calluses, bumps, or a combination thereof.

In some embodiments the method reduces skin roughness by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to a control.

In some embodiments, the method reduces wrinkle depth volume. In some embodiments, wrinkle depth volume represents the total volume of wrinkle folds across a defined surface area, incorporating both depth and width of the wrinkles.

In some embodiments, the method reduces wrinkle depth volume by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to a control.

In some embodiments, the method reduces wrinkle depth value. In some embodiments, wrinkle depth value represents the perpendicular depth of a wrinkle relative to the skin surface.

In some embodiments the method reduces wrinkle depth value by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to a control.

In some embodiments, skin roughness, wrinkle depth volume, and wrinkle depth value are measured using a profilometry system. A profilometry system is a three-dimensional skin analysis system configured to map surface topography. For example, the PRIMOS™ 3D skin analysis system projects a pattern of parallel lines onto the skin and a camera captures how these lines are distorted by skin surface variations.

In some embodiments, the method increases skin firmness. In some embodiments, skin firmness is assessed based on the skin's tightness and resistance to deformation. In some embodiments, the method increases skin elasticity. In some embodiments, skin elasticity is assessed based on the skin's ability to stretch and snap back to its original shape. In some embodiments, skin firmness and skin elasticity are measured by applying negative pressure to the skin and measuring deformation and recovery, such as by using a cutometer device (e.g., ElastiMeter®, Delfin Technologies®, Ltd.), which creates a vacuum to pull the skin into a probe aperture, and uses an optical sensor to measure how much the skin moves (firmness) and how fast it returns to normal (elasticity).

In some embodiments the method increases skin firmness by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to a control.

In some embodiments, the method increases skin luminosity. In some embodiments, skin luminosity is assessed by the skin's ability to reflect light. In some embodiments, skin luminosity is measured using imaging systems configured to capture standardized images under multiple lighting conditions, such as VISIA CR® imaging (Canfield Scientific®, Inc.), which is a high-resolution facial imaging system that captures standardized, multi-spectral images to analyze skin health. It uses several lighting modes to measure wrinkles, spots, pores, color variations, and bacteria.

In some embodiments the method increases skin luminosity by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to a control.

In some embodiments, the method reduces inflammation in the skin. In some embodiments, the population of EVs comprises a “tuned” cargo effective to reduce inflammation and regulate immune responses, wherein the “tuned” cargo comprises hsa-miR-143-3p, hsa-miR-21, hsa-let-7i-5p, hsa-miR-199a-3p, hsa-miR-26a, hsa-miR-29a-3p, hsa-miR-27b, hsa-miR-155-5p, hsa-miR-26b, hsa-miR-10a-5p, hsa-miR-99b, hsa-miR-146b, hsa-miR-24, hsa-miR-146a, hsa-miR-145, hsa-miR-25, hsa-miR-140-3p, or a combination thereof.

In some embodiments, the method stimulates tissue repair mechanisms in skin. In some embodiments, the population of EVs comprises a “tuned” cargo effective to stimulate tissue repair mechanisms and wound healing, wherein the “tuned” cargo comprises hsa-miR-10b, hsa-miR-21, hsa-miR-146a, hsa-miR-10a, hsa-miR-146b, hsa-miR-155, or a combination thereof.

In some embodiments, the method promotes cell proliferation, meaning an increase in the number of cells as a result of cell growth and cell division. In some embodiments, the method promotes keratinocyte proliferation in the skin. Imbalance between proliferation and differentiation of keratinocytes is a characteristic feature of major human skin diseases such as psoriasis and squamous cell carcinoma (see, Zhang, P., et al. Cell Death Dis 15, 392 (2024)). In some embodiments, the method promotes fibroblast proliferation in the skin. Fibroblasts may contribute to secretion and assembly of matrix components required for cell migration, provide signals for re-epithelialization, produce bioactive mediators promoting cellular differentiation, and regulate the immune response (see, Boraldi F, et al. Biomedicines. 2024 Jul. 17; 12(7):1586). In some embodiments, the population of EVs comprises a “tuned” cargo effective to promote cell proliferation, wherein the “tuned” cargo comprises hsa-miR-100-5p, hsa-miR-125-5p, hsa-miR-24-3p, hsa-miR-146a-5p, hsa-miR-222-3p, hsa-miR-22-3p, hsa-miR-21-5p, hsa-let-7a-5p, hsa-miR-29a-3p, or a combination thereof.

In some embodiments, the method promotes collagen production. In some embodiments, the population of EVs comprises a “tuned” cargo effective to promote collagen production, wherein the “tuned” cargo comprises hsa-miR-143-3p, hsa-miR-29a-3p, hsa-miR-21-5p, hsa-miR-140-3p, hsa-miR-25-3p, hsa-miR-21-3p, or a combination thereof.

In some embodiments, the method restores the skin barrier. The term “skin barrier”, which is also commonly referred to as the epidermal barrier, refers to the ability of the epidermis, especially the stratum corneum (SC), to provide selective permeability of exogenous and endogenous substances and to continually regulate and maintain homeostatic water content and balance in order to sustain healthy skin elasticity and physiologic desquamation (see, Del Rosso J Q, et al. J Clin Aesthet Dermatol. 2025 February; 18(2):7-15). In some embodiments, the population of EVs comprises a “tuned” cargo effective to restore the skin barrier, wherein the “tuned” cargo comprises hsa-miR-143, hsa-miR-155-5p, hsa-miR-146a, or a combination thereof.

### Methods for Treating Eczema (Dermatitis)

Eczema, which is also called dermatitis, is a term for several different types of skin swelling. Most types cause dry, itchy skin and rashes on the face, inside the elbows and behind the knees, and on the hands and feet. Scratching the skin can cause it to turn red and to swell and itch even more. The cause of eczema is unknown. It is likely caused by both genetic and environmental factors. Eczema may get better or worse over time, but it is often a long-lasting disease. People who have it may also develop environmental allergies, such as hay fever and asthma. The most common type of eczema is atopic dermatitis (see, medlineplus.gov/eczema).

In some embodiments, the present disclosure provides a method for treating a skin condition, wherein the skin condition is eczema comprising: (i) obtaining a biological sample comprising a population of extracellular vesicles (EVs) comprising a cargo composition; (ii) exposing the biological sample comprising the population of EVs comprising the cargo composition to one or more stress conditions to form a population of EVs comprising a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo, and (v) applying the composition topically to a region of the skin affected by eczema wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p; and
(ii) a signature of one or more proteins selected from the group consisting of BMP2, SOX2, IGF1, CDKN2A, TCF4, AR, LEP, TGFB2, KITLG, NOG, BMP4, DCN, STAT5A, STAT6, SFRP1, AXIN2, HEY1, 1L1B, MK167, HSP27, HSP60, HSP70, HSP90, CD9, CD63, CD81, CD82 and WNT5A.

In some embodiments, the methods described herein comprise administering, to a subject, the composition comprising a purified population of EVs comprising a “tuned” cargo topically to regions of the skin affected by eczema. In some embodiments, the administration of the composition comprising a purified population of EVs comprising a “tuned” cargo reduces one or more symptoms associated with eczema. In some embodiments, the one or more symptoms associated with eczema comprise inflammation, redness, scaling, dryness, irritation, discomfort, itchiness, or a combination thereof.

In some embodiments the method reduces the one or more symptoms associated with eczema by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to a control.

In some embodiments, the method reduces inflammation in skin affected by eczema. In some embodiments, the population of EVs comprises a “tuned” cargo effective to reduce inflammation and regulate immune responses, wherein the “tuned” cargo comprises hsa-miR-143-3p, hsa-miR-21, hsa-let-7i-5p, hsa-miR-199a-3p, hsa-miR-26a, hsa-miR-29a-3p, hsa-miR-27b, hsa-miR-155-5p, hsa-miR-26b, hsa-miR-10a-5p, hsa-miR-99b, hsa-miR-146b, hsa-miR-24, hsa-miR-146a, hsa-miR-145, hsa-miR-25, hsa-miR-140-3p, or a combination thereof.

In some embodiments, the method stimulates tissue repair mechanisms in skin affected by eczema. In some embodiments, the population of EVs comprises a “tuned” cargo effective to stimulate tissue repair mechanisms and wound healing, wherein the “tuned” cargo comprises hsa-miR-10b, hsa-miR-21, hsa-miR-146a, hsa-miR-10a, hsa-miR-146b, hsa-miR-155, or a combination thereof.

In some embodiments, the method promotes cell proliferation. In some embodiments, the method promotes keratinocyte proliferation to repair damaged epidermis. In some embodiments, the population of EVs comprises a “tuned” cargo effective to promote cell proliferation, wherein the “tuned” cargo comprises hsa-miR-100-5p, hsa-miR-125-5p, hsa-miR-24-3p, hsa-miR-146a-5p, hsa-miR-222-3p, hsa-miR-22-3p, hsa-miR-21-5p, hsa-let-7a-5p, hsa-miR-29a-3p, or a combination thereof.

In some embodiments, the method promotes collagen production. In some embodiments, the population of EVs comprises a “tuned” cargo effective to promote collagen production, wherein the “tuned” cargo comprises hsa-miR-143-3p, hsa-miR-29a-3p, hsa-miR-21-5p, hsa-miR-140-3p, hsa-miR-25-3p, hsa-miR-21-3p, or a combination thereof.

In some embodiments, the method restores the skin barrier. In some embodiments, the population of EVs comprises a “tuned” cargo effective to restore the skin barrier, wherein the “tuned” cargo comprises hsa-miR-143, hsa-miR-155-5p, hsa-miR-146a, or a combination thereof.

In some embodiments, the method increases levels of one or more miRNAs that are reduced in eczema. In some embodiments the one or more miRNAs comprise miR-29b, miR-143, or a combination thereof.

### Methods for Promoting Wound Healing

In some embodiments, the present disclosure provides a method for promoting wound healing comprising: (i) obtaining a biological sample comprising a population of extracellular vesicles (EVs) comprising a cargo composition; (ii) exposing the biological sample comprising the population of EVs comprising the cargo composition to one or more stress conditions to form a population of EVs comprising a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo, and (v) applying the composition topically to a wound, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof, and
(ii) a signature of proteins selected from the group consisting of BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82 and WNT5A.

In some embodiments, the methods described herein comprise administering the composition comprising a purified population of EVs comprising a “tuned” cargo topically to a wound of a subject. In some embodiments, the wound comprises one or more of an abrasion, a laceration, a puncture, an incision, a burn, an ulcer, a diabetic ulcer, a pressure ulcer, a venous ulcer, a lip ulcer, a mucosal wound, a gum recession, or a combination thereof.

In some embodiments, the administration of the composition comprising a purified population of EVs comprising a “tuned” cargo modulates one or more parameters associated with wound healing. In some embodiments, the one or more parameters associated with wound healing comprise increasing collagen production, increasing cell proliferation, promoting angiogenesis, promoting immune regulation and reducing inflammation, promoting tissue regeneration and wound closure, or a combination thereof.

In some embodiments the method modulates the one or more parameters associated with wound healing by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to a control.

In some embodiments, the methods described herein promote wound healing by increasing collagen production. In some embodiments, the population of EVs comprises a “tuned” cargo effective to promote collagen production, wherein the “tuned” cargo comprises hsa-miR-143-3p, hsa-miR-29a-3p, hsa-miR-21-5p, hsa-miR-140-3p, hsa-miR-25-3p, hsa-miR-21-3p, or a combination thereof.

In some embodiments, the methods described herein promote wound healing by increasing cell proliferation. In some embodiments, the population of EVs comprises a “tuned” cargo effective to promote cell proliferation, wherein the “tuned” cargo comprises hsa-miR-100-5p, hsa-miR-125-5p, hsa-miR-24-3p, hsa-miR-146a-5p, hsa-miR-222-3p, hsa-miR-22-3p, hsa-miR-21-5p, hsa-let-7a-5p, hsa-miR-29a-3p, or a combination thereof.

In some embodiments, the methods described herein promote wound healing by promoting angiogenesis. In some embodiments, the population of EVs comprises a “tuned” cargo effective to promote angiogenesis, wherein the “tuned” cargo comprises hsa-miR-143, hsa-miR-125-5p, hsa-miR-125a-5p, hsa-miR-26a-5p, hsa-miR-29a-3p, hsa-miR-222, hsa-miR-186-5p, hsa-miR-155-5p, hsa-miR-22-3p, hsa-miR-21-5p, hsa-miR-199a-3p, hsa-miR-92a-3p, hsa-miR-221, hsa-miR-27b, hsa-miR-181a-5p, hsa-let-7b, or a combination thereof.

In some embodiments, the methods described herein promote wound healing by promoting immune regulation and reducing inflammation. In some embodiments, the population of EVs comprises a “tuned” cargo effective to promote immune regulation and reducing inflammation, wherein the “tuned” cargo comprises hsa-miR-143-3p, hsa-miR-21, hsa-let-7i-5p, hsa-miR-199a-3p, hsa-miR-26a, hsa-miR-29a-3p, hsa-miR-27b, hsa-miR-155-5p, hsa-miR-26b, hsa-miR-10a-5p, hsa-miR-99b, hsa-miR-146b, hsa-miR-24, hsa-miR-146a, hsa-miR-145, hsa-miR-25, hsa-miR-140-3p, or a combination thereof.

In some embodiments, the methods described herein promote wound healing by promoting tissue regeneration and wound closure. In some embodiments, the population of EVs comprises a “tuned” cargo effective to promote tissue regeneration and wound closure, wherein the “tuned” cargo comprises hsa-miR-10b, hsa-miR-21, hsa-miR-146a, hsa-miR-10a, hsa-miR-146b, hsa-miR-155, or a combination thereof.

### Methods for Promoting Hair Health

In some embodiments, the present disclosure provides a method for promoting hair health comprising: (i) obtaining a biological sample comprising a population of extracellular vesicles (EVs) comprising a cargo composition; (ii) exposing the biological sample comprising the population of EVs comprising the cargo composition to one or more stress conditions to form a population of EVs comprising a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo; and (v) applying the composition topically to hair, scalp, eyebrows, eyelashes, or a combination thereof, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p; and
(ii) a signature of proteins selected from the group consisting of BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82 and WNT5A.

In some embodiments, the methods described herein comprise administering topically to hair, scalp, eyebrows, eyelashes, or a combination thereof of a subject, the composition comprising a purified population of EVs comprising a “tuned” cargo.

In some embodiments, the administration of the composition comprising a purified population of EVs comprising a “tuned” cargo modulates one or more parameters associated with hair health. In some embodiments, the one or more parameters associated with hair health comprise decreasing hair loss, increasing hair density, increasing the appearance of hair thickness, improving scalp health, improving hair shine, improving hair volume, or a combination thereof.

In some embodiments the method modulates the one or more parameters associated with hair health by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to a control.

In some embodiments, the administration of the composition comprising a purified population of EVs comprising a “tuned” cargo, modulates gene expression in dermal papillae cells, hair follicle stem cells, or a combination thereof. In some embodiments, the administration of the composition comprising a purified population of EVs comprising a “tuned” cargo is effective in treating or improving a scalp condition. In some embodiments, the scalp conditions comprises male pattern hair loss, alopecia, hair loss associated with menopause, hair loss associated with chemical damage, or a combination thereof.

In some embodiments, the methods described herein promote hair health by promoting hair growth and inhibiting hair loss. In some embodiments, the population of EVs comprises a “tuned” cargo effective to promote hair growth and inhibit hair loss, wherein the “tuned” cargo comprises miR-21-5p, miR-24, miR-26a-5p, miR-146a, let-7b, miR-101-3p, miR-181a-5p, miR-21-3p, miR-148b-3p, miR-486-5p, miR-103a-3p, or a combination thereof.

In some embodiments, the methods described herein promote hair health by increasing cell proliferation. In some embodiments, the methods described herein promote hair health by increasing cell proliferation in dermal papillae fibroblasts. In some embodiments, the population of EVs comprises a “tuned” cargo effective to promote cell proliferation, wherein the “tuned” cargo comprises miR-100-5p, miR-125-5p, miR-24-3p, miR-146a-5p, miR-222-3p, miR-22-3p, miR-21-5p, let-7a-5p, miR-29a-3p, or a combination thereof.

### Methods for Improving Vaginal Tissue Health

In some embodiments, the present disclosure provides a method for promoting vaginal health comprising: (i) obtaining a biological sample comprising a population of extracellular vesicles (EVs) comprising a cargo composition; (ii) exposing the biological sample comprising the population of EVs comprising the cargo composition to one or more stress conditions to form a population of EVs comprising a tuned cargo composition; (iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive; (iv) preparing a cosmetic composition containing a cosmetically acceptable carrier and about 1×10E8 to about 1×10E12, inclusive, of the EVs comprising the tuned cargo, and (v) applying the composition topically to vaginal skin, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof, and
(ii) a signature of proteins selected from the group consisting of BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82 and WNT5A.

In some embodiments, the methods described herein comprise administering topically to vaginal skin of a subject the composition comprising a purified population of EVs comprising a “tuned” cargo. In some embodiments, the administration of the composition comprising a purified population of EVs comprising a “tuned” cargo improves a vaginal or vulvovaginal condition associated with childbirth, menopause, aging, hormonal changes, tissue atrophy, dryness, irritation, loss of elasticity, decreased sensation, or a combination thereof.

In some embodiments, the administration of the composition comprising a purified population of EVs comprising a “tuned” cargo modulates one or more parameters associated with vaginal health. In some embodiments, the one or more parameters associated with vaginal health comprise tissue regeneration, increasing collage production, extracellular matrix remodeling, promoting angiogenesis, reducing inflammation, reducing vaginal dryness, increasing vaginal sensitization, or a combination thereof.

In some embodiments the method modulates the one or more parameters associated with vaginal health by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to a control.

In some embodiments, the methods described herein promote vaginal health by increasing collagen production and extracellular matrix remodeling. In some embodiments, the population of EVs comprises a “tuned” cargo effective to promote collagen production and extracellular matrix remodeling, wherein the “tuned” cargo comprises hsa-miR-143-3p, hsa-miR-29a-3p, hsa-miR-21-5p, hsa-miR-140-3p, hsa-miR-25-3p, hsa-miR-21-3p, or a combination thereof.

In some embodiments, the methods described herein promote vaginal health by promoting angiogenesis and increasing vaginal sensitization. In some embodiments, the population of EVs comprises a “tuned” cargo effective to promote promoting angiogenesis, increasing vaginal sensitization, wherein the “tuned” cargo comprises hsa-miR-143, hsa-miR-125-5p, hsa-miR-125a-5p, hsa-miR-26a-5p, hsa-miR-29a-3p, hsa-miR-222, hsa-miR-186-5p, hsa-miR-155-5p, hsa-miR-22-3p, hsa-miR-21-5p, hsa-miR-199a-3p, hsa-miR-92a-3p, hsa-miR-221, hsa-miR-27b, hsa-miR-181a-5p, hsa-let-7b, or a combination thereof.

In some embodiments, the methods described herein promote vaginal health by promoting immune regulation and reducing inflammation. In some embodiments, the population of EVs comprises a “tuned” cargo effective to promote immune regulation and reducing inflammation, wherein the “tuned” cargo comprises hsa-miR-143-3p, hsa-miR-21, hsa-let-7i-5p, hsa-miR-199a-3p, hsa-miR-26a, hsa-miR-29a-3p, hsa-miR-27b, hsa-miR-155-5p, hsa-miR-26b, hsa-miR-10a-5p, hsa-miR-99b, hsa-miR-146b, hsa-miR-24, hsa-miR-146a, hsa-miR-145, hsa-miR-25, hsa-miR-140-3p, or a combination thereof.

In some embodiments, the methods described herein promote vaginal health by promoting tissue regeneration. In some embodiments, the population of EVs comprises a “tuned” cargo effective to promote tissue regeneration, wherein the “tuned” cargo comprises hsa-miR-10b, hsa-miR-21, hsa-miR-146a, hsa-miR-10a, hsa-miR-146b, hsa-miR-155, or a combination thereof.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Tuning and Isolating Extracellular Vesicles from Aloe Vera Plants and Adipose-Derived Stem Cells

Tuning and Isolating Extracellular Vesicles from Adipose-Derived Stem Cells

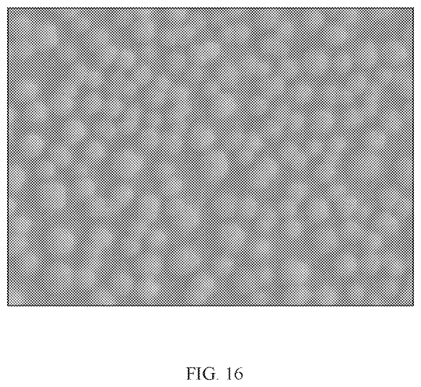

EVs were isolated from adipose-derived stem cells (ASCs) (see, FIG. 16) using a hollow-fiber cartridge containing bioreactor (Ludlow and Buehrer, 2022). The ASCs used were isolated from adipose tissue and expanded in culture (Buehrer and Cheatham, 2013).

FIG. 16 is a scanning electron microscopy (SEM) image of adipose-derived stem cell (ASC)-derived exosomes.

ASCs were cultured in a hollow fiber cartridge bioreactor (FIBERCELL BIOSYSTEMS®) to produce exosomes having increased levels of heat shock stress-response molecules as follows. Prior to seeding, the bioreactor was conditioned with complete culture medium (Gibco® DMEM/F12 media with 10% FBS) for 24 hours at 37 °C in a humidified, 5% CO₂ containing atmosphere. The bioreactor was seeded with 300×10E6 ASCs and maintained at 37 °C in a humidified, 5% CO₂ containing atmosphere. Cells were grown for 2 weeks before beginning exosome harvest.

Prior to harvesting exosome-containing medium, the bioreactor was washed 5 times with serum-free DMEM/F12 media to remove exosomes. After washing, the cells were subjected to a heat shock step as follows. The media in the bioreactor was replaced with serum-free DMEM/F12 media warmed to 41 °C, and the bioreactor was placed in a 41 °C, humidified, 5% CO₂ containing atmosphere for 1 hour. Next, the 41° C. media was replaced with the same media warmed to 37 °C, and the bioreactor was placed in a 37 °C, humidified, 5% CO₂ containing atmosphere for 48 hours. After the 48 hour incubation, the conditioned serum-free DMEM/F12 media was recovered, and in some instances, stored at −80 °C for future processing.

In separate preparations of isolated exosomes having increased levels of heat shock stress-response molecules, the same procedure as described above was followed except that the temperature of the medium used in the heat shock step was about 42 °C or about 43 °C and the time period of heat shock ranged from about 1 hour to about 3 hours.

After thawing or fresh collection of the conditioned medium described above, the exosomes were isolated from the conditioned media by centrifugation of the medium at 3000×g for 20 min at room temperature to pellet cell debris (in 50, 250, or 500 mL screw cap vessels). The clarified supernatant was collected and centrifuged at 100,000×g (Avg. RCF) for 2 hours at 4 °C. The supernatant was aspirated and the pellet(s) resuspended in minimum volume of Dulbecco's phosphate buffered saline (DPBS) (300-1000 l).

Protein and RNA concentration was estimated using a Nanodrop® (ThermoFisher®) spectrophotometer. The number of exosomes per milliliter and the exosome size were determined by Tunable Resistive Pulse Sensing (TRPS) using a qNano® (IZON®). The isolated exosomes were aliquoted into appropriate volumes into 1.5 mL screw cap tubes.

Bioreactor cultures can be maintained in this manner for up to 3 months while the ASCs retain their International Society for Cell and Gene Therapy (ISCT)-mandated mesenchymal stromal cell (MSC) characteristics.

The ISCT defines three minimum criteria to identify MSCs: (1) adherence to plastic in standard culture conditions; (2) a surface marker phenotype comprising CD105+, CD73+, CD90+, CD45−, CD34−, CD14− or CD11b−, CD79α− or CD19−, and HLA-DR−; and (3) a capability to differentiate into osteoblasts, adipocytes, and chrondroblasts in vitro (see Keating A. Mesenchymal stromal cells. Curr Opin Hematol. 2006 November; 13(6):419-25).

Tuning and Isolating Extracellular Vesicles from Aloe Vera Plants

As previously described in U.S. Non-Provisional application Ser. No. 19/270,704, Aloe vera plants were grown and sampled under the following conditions. Initially, the plants were maintained at room temperature at approximately 25 °C with ample water and light. The plants were then heat shocked by warming them to about 35 °C-45 °C in an incubator for 1-3 hours.

For leaf preparation, the inner leaf pulp containing parenchymal mesophyll cells was carefully removed to avoid collecting any outer leaf skin material and then homogenized using a Precellys® tissue homogenizer, ceramic beads and sterile PBS to disrupt the plant pulp tissue. The homogenate was transferred to a large tube and the beads were washed with sterile PBS to collect the homogenate. The homogenate was transferred for storage at −80 °C until used for extracellular vesicle (EV) isolation.

EVs (i.e., exosomes) produced by the plants were isolated from the inner leaf pulp homogenate according to the following procedure. The homogenate was diluted in PBS at a ratio of about 16 g of homogenate per 50 mL PBS. The diluted homogenate mixture was then fractionated by differential centrifugation to isolate the exosomes produced by the plants. In particular, the diluted homogenate mixture was subjected to centrifugation at 4,000×g for 20 min at room temperature to pellet cell debris. The clarified supernatant was collected and subjected to centrifugation at 120,000×g (avg. RCF or g-force) for 30 minutes at room temperature to remove any intact chloroplasts.

The subsequent clarified supernatant was brought up to 360 mL with PBS and subjected to centrifugation at 100,000×g (avg. RCF) for 2 hours at 4 °C to pellet exosomes. The supernatant was aspirated and the pellet(s) suspended in minimum volume of DPBS (3000-10000 μL). Particle diameter and concentration were assessed by tunable resistive pulse sensing (TRPS; qNano®, Izon®) using a NP150 nanopore membrane at a 47 mm stretch. The concentration of particles was standardized using multi-pressure calibration with 110 nm carboxylated polystyrene beads at a concentration of 1.2×10E13 particles/mL. The isolated exosomes were aliquoted in appropriate volumes into screw cap tubes.

### Example 2: Functional Characterization of Tuned ASC-Derived and Aloe-Derived Exosomes

### Stress Response Modulation

Heat shock protein 70 (HSP70) is a stress response molecule that has been shown to be a cargo constituent of exosomes. Modulation of stress response proteins within exosome cargo may contribute to enhanced pro-healing and regenerative properties. Heat shock proteins (HSPs) are emerging as a key area of interest relative to the biological mechanisms of aging and longevity. HSPs act as cellular protectors and have been linked to enhanced stress resistance and improved cellular function (Hu C, et al. MedComm (2020). 2022 Aug. 2; 3(3):e161).

HSPs are a group of molecular chaperones that play a crucial role in maintaining cellular homeostasis. HSPs are called “heat shock” proteins because their expression is induced in response to stressors like heat, toxins, infections, and other environmental challenges (Hu C, et al. MedComm (2020) 2022 Aug. 2; 3(3):e161).

HSPs have several key roles including protein folding and stability. HSPs assist in the proper folding of proteins, preventing misfolding and aggregation. This ensures that cellular proteins maintain their structural integrity and function optimally. HSPs are also implicated in the cellular stress response. HSPs are activated in response to various stressors, helping cells cope with adverse conditions. This stress response is vital for cellular survival and resilience. HSPs also play a role in aging and have anti-aging effects. The upregulation of heat shock proteins may contribute to delaying the aging process by preserving cellular function and reducing the accumulation of damaged proteins (Hu C, et al. MedComm (2020) 2022 Aug. 2; 3(3):e161).

Chronic oxidative stress and inflammatory stress situations (with higher levels of oxidant and inflammatory compounds and lower antioxidant and anti-inflammatory defenses) are the basis of age-related alterations of body cells. Since oxidation and inflammation are interlinked processes, and HSP70 has been shown to confer protection against the harmful effects of oxidative stress, as well as modulating the inflammatory status, it can play a role as a regulator of the rate of aging. Mechanisms affected by HSP70 that can interfere with the deleterious effects of excessive oxidative stress and chronic low-grade inflammation and that are closely related to the aging process have been detailed (Broome, C. S., et al. The FASEB Journal 20.9 (2006): 1549-1551, De Toda, I. M., (2015). Biogerontology, 16(6), 709-721, Tandara, A. A., Journal of Surgical Research, 132(1), 32-39, Kurashova, N. A. Advances in Gerontology, 10(1), 20-25).

The levels of HSP70 are higher in younger cells and tissues compared to older ones and the capacity to produce HSP70 decreases with aging, and this reduction can contribute to the accumulation of protein aggregates and impaired cellular function, which are hallmarks of aging (Heydari, A. R., et al. Experientia, 50(11), 1092-1098).

UV radiation can cause oxidative stress and inflammation in skin cells, leading to damage and premature aging. HSP70 helps to mitigate these effects by acting as a chaperone to refold damaged proteins, preventing them from aggregating and causing harm, modulating the inflammatory response, protecting cells from apoptosis, and promoting cell survival and repair (Roh, B. H., et al. Annals of dermatology, 20(4), 184, Bivik, C., et al. Carcinogenesis, 28(3), 537-544).

HSP70 has been shown to suppress UV-induced wrinkle formation, epidermal hyperplasia (increased skin thickness), and degeneration of the extracellular matrix (EMC) (Matsuda, M., et al. Journal of Investigative Dermatology, 133(4), 919-928). HSP70 has been shown to reduced fibroblast cell death, inflammation, and the activation of enzymes that degrade the ECM, like matric metalloproteinases (MMPs) and elastase (Marozzi, M., et al. International journal of molecular sciences, 22(15), 8102). Moreover, HSP70 has a role in regulating melanin synthesis, influencing skin pigmentation (Hoshino, T., et al. Journal of Biological Chemistry, 285(17), 13254-13263).

Moreover, circadian rhythms are controlled by a network of genes (clock genes) that regulate physiological and behavioral processes on a daily basis. HSP70 shows a connection to circadian genes, with evidence suggesting that HSF1, a circadian transcription factor, regulates HSP70 gene transcription. HSP70 levels can fluctuate in response to light, and this may also impact the expression of circadian genes. HSPs are also regulators of BMAL1 stability, and, as a consequence, of BMAL1-dependent transcriptional activation in the circadian clock (Tsukamoto, D., et al. Scientific Reports, 9(1), 832, Tamaru, T., et al. (2016). The Journal of Physiological Sciences, 66(4), 303-306).

Skin aging is often accompanied by disruption of circadian rhythm and abnormal expression of circadian rhythm-related genes and proteins. Misfolded tau proteins in skin potentially serve as a biomarker for tauopathies (age-related neurodegenerative decline). HSP70 may play a role in regulating tau protein solubility and preventing tau aggregation (Chinnathambi, S. (2025). Advances in protein chemistry and structural biology, 146, 161-178).

The following studies evaluated whether manufacturing methodologies could modulate HSP70 expression in both ASC-derived exosomes and Aloe-derived exosomes.

### Induction of HSP70 Protein in ASC-Derived Exosomes

To evaluate whether manufacturing methodologies modulate stress response protein expression in ASC-derived exosomes, western blot analysis was performed to assess HSP70 levels in tuned ASC-derived exosomes compared to native (not heat shocked) ASC exosomes.

The heat stock step includes culturing ASCs, wherein the culturing includes a step of heat shocking the ASCs in a serum-free culture media by increasing the culture temperature to about 41 °C-43 °C for about 1-3 hours. The process may further include culturing the ASCs in the serum-free culture medium at a temperature of about 36 °C-38 °C for about 24-72 hours subsequent to the step of heat shocking.

The results indicated a significant upregulation in exosomal HSP70 relative to (3-actin in the tuned (heat-shocked) ASC-derived exosomes compared to native (not heat shocked) ASC-derived exosomes (see, FIG. 17).

FIG. 17 is a bar graph of quantified western blot data that shows the amount of HSP70 protein relative to actin protein in two separate preparations of exosomes: exosomes secreted by ASCs cultured at 37 °C without a heat shock step (native); and exosomes secreted by ASCs subjected to a 2 hour heat shock step at 43 °C (EXO3).

The data in FIG. 17 show that there is a significant up-regulation in exosomal HSP70 relative to actin in the heat shock exosomes compared to the exosomes from the ASCs cultured without the heat shock step.

In a second experiment, two different ASC sources (Source 1 and Source 2) were used and their respective exosome preparations were analyzed to compare the effect of manufacturing methods on the two different ASC sources used to isolate exosomes. Two different manufacturing methods (heat shocked, as described in Example 1 vs. not heat shocked) were assessed. The results were compared to determine whether the production of heat shock stress response molecules was different between tuned (heat shocked) and native (not heat shocked) exosomes across different sources. Four test materials were evaluated:
(1) ASC source 1, native exosomes (no heat shocking);
(2) ASC source 1, tuned (heat shocked) exosomes;
(3) ASC source 2, native exosomes (no heat shocking); and
(4) ASC source 2, tuned (heat shocked) exosomes.

HSP70 protein expression was normalized to 3-actin as a loading control.

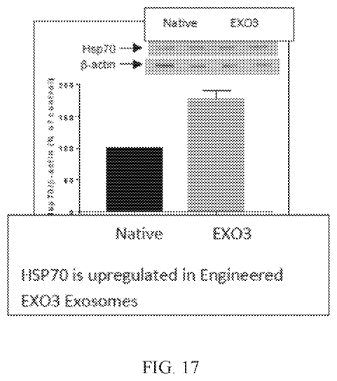

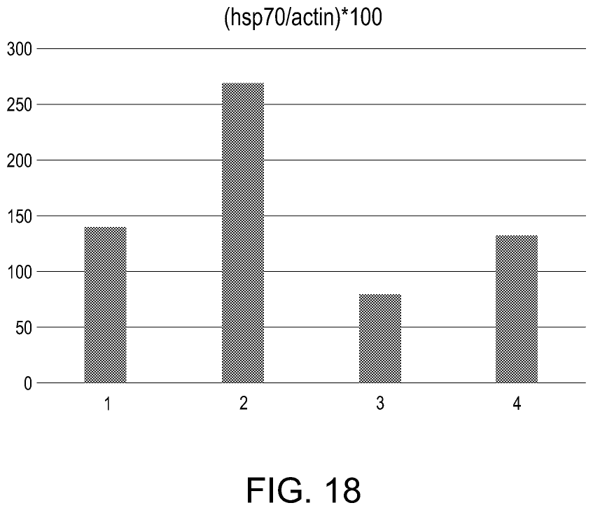

FIG. 18 shows a western blot quantification assessing HSP70 levels in these four preparations. The results demonstrate differential HSP70 protein expression across the four conditions, with tuned exosome preparations showing upregulation of HSP70 compared to their respective no heat shock standard controls (see, FIG. 18). Preparation (2) had the greatest effect on HSP70 expression.

### Induction of HSP70 Protein in Aloe-Derived Exosomes

To evaluate the effect of different manufacturing methodologies on the relative change in HSP70 mRNA transcript levels in Aloe-derived exosomes, exosomes were isolated from Aloe vera plants and qPCR was performed to quantify HSP70 gene expression.

Isolated exosomes were characterized using the following procedures. To determine and compare changes in heat shock stress response molecules in the isolated Aloe-derived exosomes, total RNA was isolated from exosomes derived from native Aloe vera plants NATIVE EXOSOMES (not heat shocked), METHOD A (Aloe-derived exosomes isolated after heat shock at 35 °C for 2 hours), METHOD B (Aloe-derived exosomes isolated after heat shock at 45 °C for 3 hours), and METHOD C (which included a 24-hour recovery period after METHOD B).

2×10E10 exosomes from each treatment were used to isolate total RNA using a microRNeasy® kit from Qiagen®. Total RNA was quantified spectrophometrically using a Nanodrop® (ThermoFisher®) spectrophotometer. cDNA was prepared using 300 ng total RNA per reaction in 20 microliter final volume and qPCR performed using 10 ng of cDNA per triplicate, primers for HSP70 and 18s genes (housekeeping gene), and SYBR Green master mix in a final volume of 15 microliters. Relative gene expression was determined by the Ct method using 18s gene expression as the internal housekeeping gene and comparisons made to the NATIVE control exosomes.

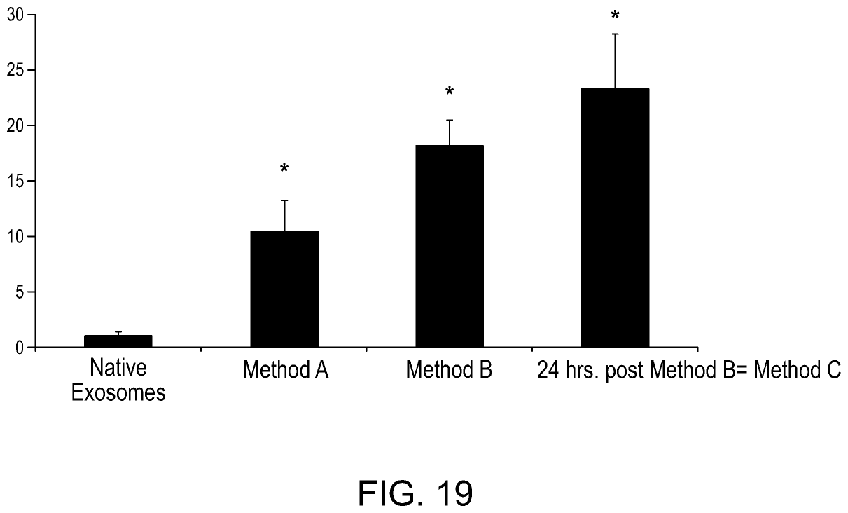

FIG. 19 is a bar graph showing the relative change in HSP70 mRNA transcript levels in exosomes isolated from Aloe vera plants for each of these samples: non-heat shocked, native Aloe-derived exosomes (Native), Aloe-derived exosomes isolated after heat shock at 35 °C for 2 hours (METHOD A), Aloe-derived exosomes isolated after heat shock at 45 °C for 3 hours (METHOD B), Aloe-derived exosomes isolated 24 hours after a 3-hour heat shock at 45 °C (METHOD C). Values shown are Relative Quantification values determined by the Ct method (Fold Change) relative to the NATIVE exosome values. Statistical significance compared to the NATIVE sample was determined using student's t-test and a p-value <0.05, significance is denoted by a “*”.

As can be seen in FIG. 19, induction of a heat shock stress in both METHOD A and METHOD B increased the transcript level of the stress response molecule, HSP70, relative to that for NATIVE plants. The transcript level of the METHOD A sample was less than the transcript level of the METHOD B sample. The data show a 10× increase in transcript level of the stress response molecule HSP70 after a heat shock of 35 °C in comparison to the NATIVE exosome sample. Transcript level of HSP70 after METHOD B was about 17× that for NATIVE exosomes, and transcript level of HSP70 after METHOD C was about 22× that for NATIVE exosomes.

As shown in FIG. 19, the increase in stress response molecules was maintained for at least 24 hours for the METHOD C sample. The data indicate that stress response molecules are significantly increased in isolated exosomes by subjecting the aloe vera plant to different exosome manufacturing methods, and that these effects persist for at least 24 hours.

### Collagen Production

### Rationale

Collagen production is associated with wound healing and skin regeneration. Evaluating the effects of tuned exosomes on collagen synthesis in fibroblasts provides insight into the functional activity of exosome preparations.

### Homogeneous Time Resolved Fluorescence (HTRF) ELISA in Dermal Fibroblasts and Periodontal Ligament Fibroblasts

Cisbio® Bioassays (a Revvity® company) HTRF ELISA is a rapid, no-wash, immunoassay technology combining fluorescence resonance energy transfer and time-resolved measurements. It measures interactions by monitoring energy transfer between a donor and acceptor label.

An HTRF ELISA was performed to measure type I collagen production in human dermal fibroblasts (DF) and human periodontal ligament fibroblasts (HPLF). A dilution factor of 30 was applied to the cell samples. Cells were allowed to attach overnight. Plated cells were treated with one of the following for 1 hour:
(1) no treatment,
(2) TGFβ at 20 ng/ml,
(3) tuned ASC-derived exosomes at 5 μg/ml,
(4) native ASC-derived exosomes at 5 μg/ml,
(5) insulin-like growth factor (IGF) at 10 ng/ml, or
(6) platelet derived growth factor (PDGF) at 100 ng/ml.

Cells were then lysed using the lysing reagent (30 minutes).

After transfer of the supernatant of each well into the ELISA plate supplied with the kit, anti-human Pro-Collagen antibody labeled with Europium cryptate (donor) and anti-human pro-collagen antibody labeled with d2 (acceptor) were added. When the dyes are in close proximity, the excitation of the donor with a light source (laser or flash lamp) triggers a fluorescence resonance energy transfer (FRET) towards the acceptor, which then fluoresces at a specific wavelength (665 nm). Signal intensity is proportional to the number of antigen-antibody complexes formed and therefore to the human pro-collagen concentration, and anti-IP-1 Mab are added and incubated for 3 hours.

Collagen concentrations were determined by measuring optical density at 665 nm and compared to a standard curve of the human pro-collagen standard.

Table 5 below presents collagen production data in dermal fibroblasts and human periodontal ligament fibroblasts.

TABLE 5
Collagen Production in Dermal Fibroblasts
and Human Periodontal Ligament Fibroblasts

| | | | | Mean Collagen | |
|---|---|---|---|---|---|
| | Test | | | Concentration | % |
| Compound | Concentration | | Cell Type | (ng/ml) | Change |
| No Treatment | — | | DF | 2677.47 | 0 |
| TGF beta | 20 | ng/ml | DF | 2409.01 | −10.03 |
| Tuned exosome | 5 | μg/ml | DF | 3706.76 | 38.44 |
| Native Exosome | 5 | μg/ml | DF | 2748.27 | 2.64 |
| IGF | 10 | ng/ml | DF | 2822.99 | 5.44 |
| PDGF | 100 | ng/ml | DF | 2846.89 | 6.33 |
| No Treatment | — | | HPLF | 856.53 | 0 |
| TGF beta | 20 | ng/ml | HPLF | 1461.73 | 70.66 |
| Tuned exosome | 5 | μg/ml | HPLF | 2949.95 | 244.41 |
| Native Exosome | 5 | μg/ml | HPLF | 2812.77 | 228.39 |
| IGF | 10 | ng/ml | HPLF | 985.97 | 15.11 |
| PDGF | 100 | ng/ml | HPLF | 717.72 | −16.21 |

In dermal fibroblasts, tuned exosomes at 5 μg/ml produced the highest collagen concentration at 3706.76 ng/ml, representing a 38.44% increase over the no treatment control. Native exosomes at 5 μg/ml produced collagen levels similar to the no treatment control.

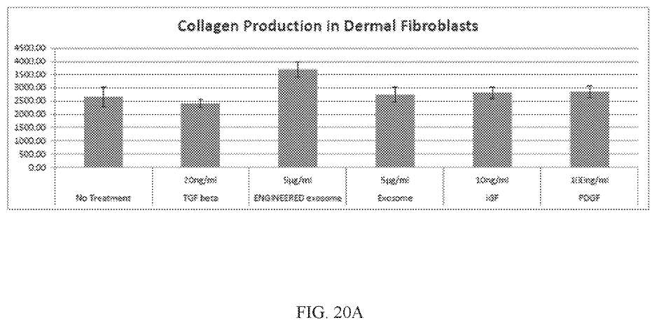

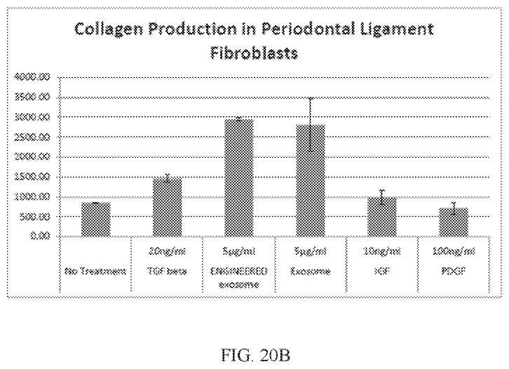

In human periodontal ligament fibroblasts, tuned exosomes at 5 μg/ml produced the highest collagen concentration at 2949.95 ng/ml, representing a 244.41% increase over the no treatment control. Native ASC-derived exosomes at 5 μg/ml also substantially increased collagen production in HPLF by 228.39% (see, FIGS. 20A-20B).

FIG. 20A and FIG. 20B are bar graphs depicting collagen production by dermal fibroblasts (DF) (FIG. 20A)) and by periodontal ligament fibroblasts (PDLFs) under these six test conditions.

FIG. 20A is a graph showing the amount of collagen I production in dermal fibroblasts after a 48 hour incubation with a media control (“no treatment”), various growth factors (IGF, PDGF, TGF beta), or exosomes secreted from cells cultured with (“engineered exosome”) or without a heat shock (“exosome”) step. Values shown on the Y axis are ng/ml of collagen.

FIG. 20B is a graph showing the amount of collagen I production in periodontal ligament fibroblasts after a 48 hour incubation with a media control (“no treatment”), various growth factors (IGF, PDGF, TGF beta), or exosomes secreted from cells cultured with (“engineered exosome”) or without a heat shock (“exosome”) step. Values shown on the Y axis are ng/ml of collagen.

The results show that treatment of both PDLFs and DFs with the isolated ASC-derived exosomes from the heat shocked cells increased collagen I production, compared to a control (isolated exosomes prepared from cells that were not subjected to a heat shock step). In addition, the increase in collagen I production of the PDLFs and DFs induced by the isolated exosomes from the heat shocked cells surpassed that of the individual growth factors.

### Effects of Aloe-Derived Exosome Manufacturing Methodologies on Collagen Production

An experiment was performed to determine if isolated exosomes prepared from Aloe vera plants using various manufacturing methodologies versus native aloe exosomes could induce collagen I synthesis in human dermal fibroblasts. For the assessment of collagen synthesis, human dermal fibroblast cells (10,000 cells/well) were treated for 48 hours with one of media only, 20 ng/ml TGFβ-1, or isolated exosomes (1×10E8 particles/mL) isolated according to the following manufacturing methods: isolated after heat shock at 35 °C for 2 hours (Method EXO A); isolated after heat shock at 45 °C for 3 hours (Method EXO B); and isolated 24 hours after a 3-hour heat shock at 45 °C (Method EXO C). After 48 hours, the conditioned medium was removed, clarified by centrifugation, and diluted into a procollagen I C-peptide HTRF ELISA (Cisbio® Bioassays) assay.

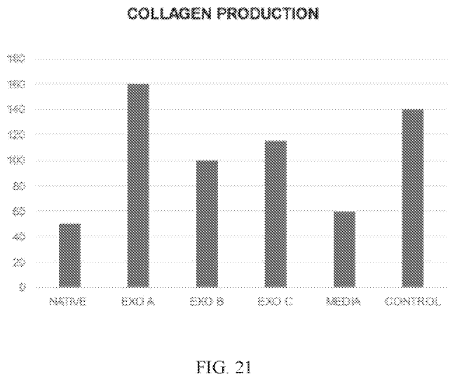

Results are shown in FIG. 21.

The results show that treatment with the isolated tuned Aloe-derived exosomes increased collagen I production of human fibroblasts. Collagen I production was increased by 42-78% depending upon which manufacturing method was used for all three tuned Aloe vera plant exosome types relative to media alone. Native exosomes from Aloe vera plants did not increase collagen I production. The highest collagen I production was measured for exosomes obtained using manufacturing methodology EXO A.

### Tuned Exosome Effects on Collagen Production in Primary Human Fibroblasts

To determine the effects of ASC-derived exosomes and the manufacturing microenvironment on collagen I synthesis in primary human fibroblasts (PHFs), serum-free conditioned medium from vehicle, TGF beta-1, native ASC-derived exosomes, or tuned ASC-derived exosomes-treated PHFs was tested in a procollagen I C-peptide ELISA (TaKaRa®). PHFs were treated for 48 hours with increasing concentrations of native ASC-derived exosomes (1 μg, 3 μg, and 10 μg), and with tuned ASC-derived exosomes at 5 μg. The conditioned medium was removed, clarified by centrifugation, and diluted for ELISA analysis.

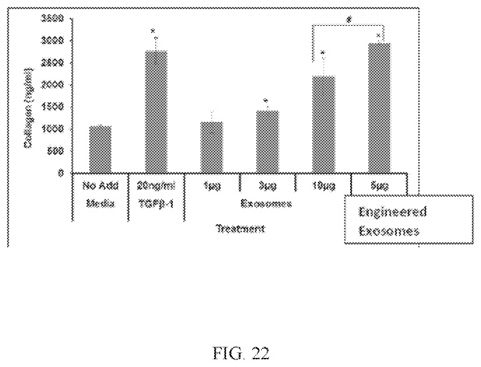

Results are shown in FIG. 22. PHFs treated with tuned ASC-derived exosomes significantly increased collagen I production to the same level as that induced by TGFβ-1. Tuned ASC-derived exosomes were significantly more active than native ASC-derived exosomes. Tuned ASC-derived exosomes at 5 μg produced the highest collagen production, exceeding the TGFβ-1 positive control (see, FIG. 22).

### Cytokine Modulation

### Rationale

Inflammatory cytokines play a central role in the pathogenesis of chronic wounds and inflammatory skin conditions. Lipopolysaccharide (LPS), a major component of the outer membrane of Gram-negative bacteria, is known to induce a strong immune response in mammalian cells, promoting the secretion of inflammatory cytokines. Evaluating the ability of tuned exosome preparations to modulate inflammatory cytokine production in both peripheral blood mononuclear cells (PBMCs) and primary human fibroblasts provides insight into the immunomodulatory properties of these preparations and their potential therapeutic application in wound healing and inflammatory skin conditions.

### Materials and Methods

An experiment was performed to determine if isolated Aloe-derived exosomes compared to native Aloe-derived exosomes could inhibit production of inflammatory cytokine IL-6 protein secretion in human peripheral blood mononuclear cells (PBMCs).

To evaluate the anti-inflammatory impact of treatment with isolated native Aloe-derived exosomes and tuned Aloe-derived exosomes, human PBMCs were concomitantly exposed to LPS (1 μg/ml) and exosomes isolated according to the following manufacturing methods: native Aloe-derived exosomes (no heat shocking), or tuned Aloe-derived exosomes manufactured by Method 1, Method 2, Method 3, or Method 4 as described below:
Method 1: Exosomes isolated after exposure to 35 °C for 2 hours;
Method 2: Exosomes isolated after exposure to 35 °C for 2 hours, followed by 45 °C for 3 hours;
Method 3: Exosomes isolated after exposure to 35 °C for 2 hours, followed by 45 °C for 3 hours, followed by 18-25 °C for 6 hours; and
Method 4: Exosomes isolated after exposure to 35 °C for 2 hours, followed by 45 °C for 3 hours, followed by 18-25 °C for 24 hours.

Dexamethasone, a synthetic glucocorticoid known to reduce inflammation in response to LPS, was included as a control. To measure inflammatory response, multiplexed quantification of secreted cytokine protein levels for the inflammatory cytokine IL-6 was performed using MagPix® and Milliplex® assay kits.

Milliplex® assay kits are commercially available, bead-based multiplex immunoassay systems that include sets of microspheres coated with capture antibodies specific to target proteins, along with detection antibodies and reagents for simultaneous quantification of multiple analytes within a single biological sample. The MagPix® system is a compatible detection instrument that analyzes these assays by identifying bead populations and measuring associated fluorescent signals.

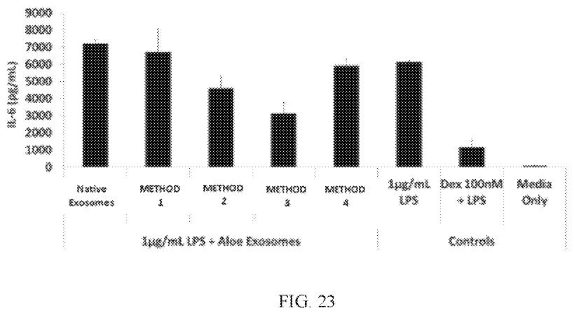

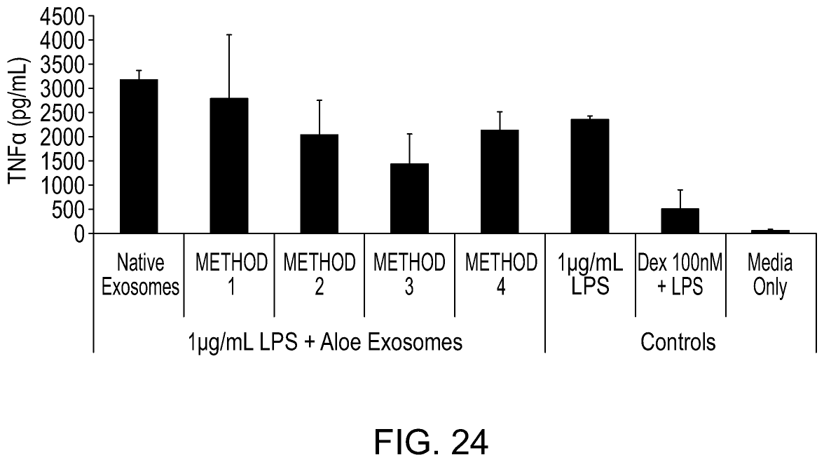

PBMCs were seeded in 96-well plates and incubated overnight prior to treatments. Cells were treated as follows: (1) without LPS and without exosomes (Media Only); (2) with 1 μg/ml LPS and without exosomes; (3) with 1 μg/ml LPS and 100 nM dexamethasone; and (4) with 1 μg/ml LPS in combination with Aloe-derived exosomes (1×10E8 particles/mL). Results are shown in FIG. 23 for IL-6 and FIG. 24 for TNFα.

### IL-6 Inhibition

FIG. 23 is a bar graph showing IL-6 secretion in human peripheral blood mononuclear cells (PBMCs) treated with (i) Lipopolysaccharide (LPS) (1 μg/ml); (ii) native aloe exosomes (no heat shocking), (iii) tuned Aloe-derived exosomes manufactured by (1) Method 1, (2) Method 2, (3) Method 3, or (4) Method 4. Dexamethosone 100 nM was included as a control.

The results showed that Method 1 promoted the greatest level of IL-6 secretion.

Treatment with tuned Aloe-derived exosomes isolated by Method 2 and Method 3 provided decreased production of IL-6. Tuned Aloe-derived exosomes isolated by Method 3 provided the largest decrease in production of IL-6, which most closely approached the IL-6 production decrease achieved by treatment with 100 nM dexamethasone.

The results indicated a significant elevation in IL-6 (87-fold) protein secretion in PBMCs induced by LPS at 1 μg/ml. The elevation was reduced by the isolated tuned exosomes by about 50%. The data indicated that tuned Aloe-derived exosomes can inhibit the production of inflammatory cytokines such as IL-6 that act locally to recruit monocytes to the site of inflammation.

### TNF-α Inhibition

FIG. 24 shows TNF-α secretion in human peripheral blood mononuclear cells (PBMCs) treated with Lipopolysaccharide (LPS) (1 μg/ml) and exosomes isolated according to the following manufacturing methods: native Aloe-derived exosomes (no heat shocking), or tuned Aloe-derived exosomes manufactured by Method 1 (exosomes isolated after exposure to 35 °C for 2 hours), Method 2 (exosomes isolated after exposure to 35 °C for 2 hours, followed by 45 °C for 3 hours), Method 3 (exosomes isolated after exposure to 35 °C for 2 hours, followed by 45 °C for 3 hours, followed by 18-25 °C for 6 hours), or Method 4 (exosomes isolated after exposure to 35 °C for 2 hours, followed by 45 °C for 3 hours, followed by 18-25 °C for 24 hours). Dexamethosone was included as a control. Method 1 promoted the greatest level of TNF-α secretion.

Treatment with Aloe-derived exosomes isolated by Method 2 and Method 3 provided decreased production of TNF-α when compared to native exosomes with no treatment. The largest decrease in production of TNF-α was provided by treatment with exosomes isolated according to Method 3.

The results showed that LPS at 1 μg/ml induced a significant elevation in TNF-α (62-fold) protein secretion in PBMCs. The elevation of TNF-α was reduced by the isolated tuned Aloe-derived exosomes by about 40%. The data indicated that the isolated tuned Aloe-derived exosomes can inhibit the production of inflammatory cytokines such as TNF-α that act locally to recruit monocytes to the site of inflammation

### Example 3: Characterization of Cargo Signature in ASC-Derived Exosomes

miRNA Cargo Characterization

### Rationale

Exosomes derived from ASCs contain miRNA cargo that mediates therapeutic effects in skin and hair applications. Characterization of the miRNA profile in these exosomes provides insight into the molecular mechanisms underlying their biological activity and enables identification of miRNA species with functional relevance to collagen production, skin barrier function, immune regulation, wound healing, angiogenesis, melanogenesis, photoaging protection, and hair growth.

### Materials and Methods

Human follicle dermal papilla cells (HFDPCs) sourced from the temple skin of a 50-year-old Caucasian female donor (PromoCell™, Cat #C-12071) were seeded into culture plates. The adherent cells were treated with test materials comprising native (non-heat shocked) and tuned (heat shocked) ASC-derived exosomes.

Test materials were: (1) ASC-derived exosomes isolated from cells cultured at 37 °C and subjected to a 2 hour heat shock step at 43 °C at varying concentrations, (2) untreated, positive control (4% fetal bovine serum (FBS) media), and (3) vehicle (phosphate-buffered saline (PBS)) control.

Following exposure to the test materials for 1 hour, fresh maintenance media was added and the cells were incubated for 24 hours or 72 hours. Total RNA was then isolated and cDNA was synthesized. Cytotoxicity was assessed via LDH assay to confirm that the test materials did not compromise cell viability.

miRNA sequencing was performed to identify and quantify miRNA species present in the exosomes. Read counts were obtained for each identified miRNA; multimap-adjusted read counts were calculated to account for miRNAs mapping to multiple genomic locations

### Results

miRNA sequencing analysis identified a total of 409 unique miRNA species in heat-shocked (tuned) ASC-derived exosomes compared to 268 unique miRNA species in non-heat-shocked (native) ASC-derived exosomes. The heat-shocked ASC-derived exosomes contained miRNA species that were not detected in the non-heat-shocked ASC-derived exosomes.

The following table (Table 6) presents the complete miRNA cargo profile comparison between heat-shocked ASC-derived exosomes and non-heat-shocked ASC-derived exosomes, indicating the presence (1) or absence (0) of each miRNA species in each population.

TABLE 6
miRNA Cargo Comparison Between Heat-Shocked
and Non-Heat-Shocked ASC-derived Exosomes

| | Heat | Non-Heat | | Heat | Non-Heat |
|---|---|---|---|---|---|
| | Shocked | Shocked | | Shocked | Shocked |
| | ASC-derived | ASC-derived | | ASC-derived | ASC-derived |
| miRNA | Exosomes | Exosomes | miRNA | Exosomes | Exosomes |
| hsa-let-7a-3p | 1 | 1 | hsa-miR-320c | 1 | 1 |
| hsa-let-7a-5p | 1 | 1 | hsa-miR-320d | 1 | 1 |
| hsa-let-7b-3p | 1 | 0 | hsa-miR-323a-3p | 1 | 1 |
| hsa-let-7b-5p | 1 | 1 | hsa-miR-323b-3p | 1 | 1 |
| hsa-let-7c | 1 | 1 | hsa-miR-324-3p | 1 | 0 |
| hsa-let-7c-5p | 1 | 1 | hsa-miR-328-3p | 1 | 0 |
| hsa-let-7d-3p | 1 | 1 | hsa-miR-329-3p | 1 | 1 |
| hsa-let-7d-5p | 1 | 1 | hsa-miR-330-3p | 1 | 1 |
| hsa-let-7e-5p | 1 | 1 | hsa-miR-331-3p | 1 | 0 |
| hsa-let-7f-2-3p | 1 | 0 | hsa-miR-331-5p | 1 | 1 |
| hsa-let-7f-5p | 1 | 1 | hsa-miR-335-3p | 1 | 1 |
| hsa-let-7g-5p | 1 | 1 | hsa-miR-335-5p | 1 | 1 |
| hsa-let-7i-5p | 1 | 1 | hsa-miR-337-3p | 1 | 0 |
| hsa-miR-100-5p | 1 | 1 | hsa-miR-337-5p | 1 | 0 |
| hsa-miR-101-3p | 1 | 1 | hsa-miR-339-3p | 1 | 1 |
| hsa-miR-10395-3p | 1 | 0 | hsa-miR-339-5p | 1 | 1 |
| hsa-miR-10399-5p | 1 | 0 | hsa-miR-33a-3p | 1 | 0 |
| hsa-mR-103a-3p | 1 | 1 | hsa-miR-33b-3p | 1 | 0 |
| hsa-miR-10527-5p | 1 | 0 | hsa-miR-340-3p | 1 | 0 |
| hsa-miR-106a-5p | 1 | 0 | hsa-miR-340-5p | 1 | 1 |
| hsa-miR-106b-3p | 1 | 1 | hsa-miR-342-3p | 1 | 1 |
| hsa-miR-106b-5p | 1 | 1 | hsa-miR-342-5p | 1 | 0 |
| hsa-miR-107 | 1 | 0 | hsa-miR-345-5p | 1 | 0 |
| hsa-miR-10a-3p | 1 | 0 | hsa-miR-34a-3p | 1 | 0 |
| hsa-miR-10a-5p | 1 | 1 | hsa-miR-34a-5p | 1 | 0 |
| hsa-miR-10b-3p | 1 | 1 | hsa-miR-34b-3p | 1 | 0 |
| hsa-miR-10b-5p | 1 | 1 | hsa-miR-34c-3p | 1 | 0 |
| hsa-miR-1180-3p | 1 | 1 | hsa-miR-34c-5p | 1 | 1 |
| hsa-miR-1197 | 1 | 0 | hsa-miR-3605-3p | 1 | 0 |
| hsa-miR-122-5p | 1 | 1 | hsa-miR-3605-5p | 1 | 0 |
| hsa-miR-1226-3p | 1 | 0 | hsa-miR-3613-5p | 1 | 1 |
| hsa-miR-1238-3p | 1 | 0 | hsa-miR-361-3p | 1 | 0 |
| hsa-miR-124-3p | 1 | 0 | hsa-miR-3614-5p | 1 | 0 |
| hsa-miR-1246 | 1 | 1 | hsa-miR-3615 | 1 | 0 |
| hsa-miR-1247-5p | 1 | 0 | hsa-miR-361-5p | 1 | 1 |
| hsa-miR-125a-3p | 1 | 1 | hsa-miR-3622a-5p | 1 | 0 |
| hsa-miR-125a-5p | 1 | 1 | hsa-miR-362-5p | 1 | 0 |
| hsa-miR-125b-1-3p | 1 | 1 | hsa-miR-365a-3p\|hs | 1 | 1 |
| hsa-miR-125b-2-3p | 1 | 1 | hsa-miR-365a-5p | 1 | 0 |
| hsa-miR-125b-5p | 1 | 1 | hsa-miR-3691-5p | 1 | 0 |
| hsa-miR-1262 | 1 | 0 | hsa-miR-369-3p | 1 | 0 |
| hsa-miR-126-3p | 1 | 1 | hsa-miR-369-5p | 1 | 0 |
| hsa-miR-126-5p | 1 | 0 | hsa-miR-370-3p | 1 | 1 |
| hsa-miR-1269b | 1 | 0 | hsa-miR-374a-3p | 1 | 1 |
| hsa-miR-1271-5p | 1 | 1 | hsa-miR-374a-5p | 1 | 1 |
| hsa-miR-127-3p | 1 | 1 | hsa-miR-374b-5p | 1 | 1 |
| hsa-miR-127-5p | 1 | 1 | hsa-miR-376c-3p | 1 | 0 |
| hsa-miR-128-1-5p | 1 | 0 | hsa-miR-377-3p | 1 | 0 |
| hsa-miR-128-3p | 1 | 1 | hsa-miR-377-5p | 1 | 0 |
| hsa-miR-1287-5p | 1 | 0 | hsa-miR-378a-3p | 1 | 1 |
| hsa-miR-1290 | 1 | 1 | hsa-miR-378c | 1 | 0 |
| hsa-miR-129-5p | 1 | 1 | hsa-miR-378d | 1 | 0 |
| hsamiR-1299 | 1 | 1 | hsa-miR-378g | 1 | 0 |
| hsa-miR-1301-3p | 1 | 1 | hsa-miR-378i | 1 | 1 |
| hsa-miR-1307-3p | 1 | 1 | hsa-miR-379-3p | 1 | 0 |
| hsa-miR-1307-5p | 1 | 1 | hsa-miR-379-5p | 1 | 1 |
| hsa-miR-130a-3p | 1 | 0 | hsa-miR-380-3p | 1 | 0 |
| hsa-miR-130b-3p | 1 | 0 | hsa-miR-381-3p | 1 | 1 |
| hsa-miR-130b-5p | 1 | 1 | hsa-miR-382-3p | 1 | 1 |
| hsa-miR-132-3p | 1 | 1 | hsa-miR-382-5p | 1 | 1 |
| hsa-miR-132-5p | 1 | 0 | hsa-miR-3909 | 1 | 1 |
| hsa-miR-1343-3p | 1 | 0 | hsa-miR-3912-3p | 1 | 0 |
| hsa-miR-134-3p | 1 | 0 | hsa-miR-3928-3p | 1 | 1 |
| hsa-miR-134-5p | 1 | 1 | hsa-miR-409-3p | 1 | 1 |
| hsa-miR-136-3p | 1 | 1 | hsa-miR-409-5p | 1 | 1 |
| hsa-miR-136-5p | 1 | 0 | hsa-miR-411-3p | 1 | 0 |
| hsa-miR-138-5p | 1 | 0 | hsa-miR-411-5p | 1 | 1 |
| hsa-miR-1-3p | 1 | 1 | hsa-miR-412-5p | 1 | 1 |
| hsa-miR-140-3p | 1 | 1 | hsa-miR-421 | 1 | 1 |
| hsa-miR-140-5p | 1 | 1 | hsa-miR-423-3p | 1 | 1 |
| hsa-miR-142-5p | 1 | 1 | hsa-miR-423-5p | 1 | 1 |
| hsa-miR-143-3p | 1 | 1 | hsa-miR-424-3p | 1 | 1 |
| hsa-miR-143-5p | 1 | 1 | hsa-miR-424-5p | 1 | 1 |
| hsa-miR-144-3p | 1 | 1 | hsa-miR-425-3p | 1 | 0 |
| hsa-miR-144-5p | 1 | 1 | hsa-miR-425-5p | 1 | 0 |
| hsa-miR-145-3p | 1 | 1 | hsa-miR-4286 | 1 | 0 |
| hsa-miR-145-5p | 1 | 1 | hsa-miR-431-5p | 1 | 0 |
| hsa-miR-1468-5p | 1 | 1 | hsa-miR-4324 | 1 | 0 |
| hsa-miR-146a-5p | 1 | 1 | hsa-miR-432-5p | 1 | 1 |
| hsa-miR-146b-3p | 1 | 1 | hsa-miR-4326 | 1 | 1 |
| hsa-miR-146b-5p | 1 | 1 | hsa-miR-433-3p | 1 | 0 |
| hsa-miR-148a-3p | 1 | 1 | hsa-miR-433-5p | 1 | 0 |
| hsa-miR-148a-5p | 1 | 1 | hsa-miR-450a-2-3p | 1 | 0 |
| hsa-miR-148b-3p | 1 | 1 | hsa-miR-450a-5p | 1 | 1 |
| hsa-miR-148b-5p | 1 | 0 | hsa-miR-450b-5p | 1 | 1 |
| hsa-miR-149-5p | 1 | 1 | hsa-miR-4510 | 1 | 1 |
| hsa-miR-151a-3p | 1 | 1 | hsa-miR-451a | 1 | 1 |
| hsa-miR-151a-5p | 1 | 1 | hsa-miR-4524a-5p | 1 | 0 |
| hsa-miR-151a-5p\|hs | 1 | 1 | hsa-miR-452-5p | 1 | 1 |
| hsa-miR-151b | 1 | 1 | hsa-miR-455-3p | 1 | 1 |
| hs-mir-151b | 1 | 0 | hsa-miR-455-5p | 1 | 1 |
| hsa-miR-152-3p | 1 | 1 | hsa-miR-4636 | 1 | 0 |
| hsa-miR-155-5p | 1 | 1 | hsa-miR-4660 | 1 | 0 |
| hsa-miR-15a-5p | 1 | 1 | hsa-miR-4661-5p | 1 | 0 |
| hsa-miR-15b-3p | 1 | 0 | hsa-miR-4662a-5p | 1 | 0 |
| hsa-miR-15b-5p | 1 | 1 | hsa-miR-4676-3p | 1 | 1 |
| hsa-miR-16-2-3p | 1 | 1 | hsa-miR-4677-3p | 1 | 0 |
| hsa-miR-16-5p | 1 | 1 | hsa-miR-4717-3p | 1 | 0 |
| hsa-miR-17-5p | 1 | 0 | hsa-miR-4722-5p | 1 | 0 |
| hsa-miR-181a-2-3p | 1 | 0 | hsa-miR-4772-5p | 1 | 0 |
| hsa-miR-181a-3p | 1 | 0 | hsa-miR-4773 | 1 | 0 |
| hsa-miR-181a-5p | 1 | 1 | hsa-miR-4775 | 1 | 1 |
| hsa-miR-181b-3p | 1 | 1 | hsa-miR-4781-3p | 1 | 0 |
| hsa-miR-181b-5p | 1 | 1 | hsa-miR-4798-5p | 1 | 0 |
| hsa-miR-181c-3p | 1 | 1 | hsa-miR-484 | 1 | 1 |
| hsa-miR-181c-5p | 1 | 0 | hsa-miR-485-3p | 1 | 0 |
| hsa-miR-181d-5p | 1 | 1 | hsa-miR-485-5p | 1 | 1 |
| hsa-miR-182-5p | 1 | 1 | hsa-miR-486-5p | 1 | 1 |
| hsa-miR-183-5p | 1 | 0 | hsa-miR-487a-3p | 1 | 0 |
| hsa-miR-184 | 1 | 1 | hsa-miR-487b-3p | 1 | 0 |
| hsa-miR-1843 | 1 | 0 | hsa-miR-493-3p | 1 | 1 |
| hsa-miR-185-3p | 1 | 0 | hsa-miR-493-5p | 1 | 1 |
| hsa-miR-185-5p | 1 | 1 | hsa-miR-494-3p | 1 | 1 |
| hsa-miR-186-5p | 1 | 1 | hsa-miR-495-3p | 1 | 0 |
| hsa-miR-1908-5p | 1 | 0 | hsa-miR-5000-3p | 1 | 0 |
| hsa-miR-190b-5p | 1 | 0 | hsa-miR-500a-3p | 1 | 1 |
| hsa-miR-191-5p | 1 | 1 | hsa-miR-500b-3p | 1 | 0 |
| hsa-miR-192-5p | 1 | 1 | hsa-miR-5010-3p | 1 | 0 |
| hsa-miR-193a-5p | 1 | 1 | hsa-miR-501-3p | 1 | 1 |
| hsa-miR-193b-5p | 1 | 1 | hsa-miR-502-3p | 1 | 0 |
| hsa-miR-194-5p | 1 | 0 | hsa-miR-503-5p | 1 | 1 |
| hsa-miR-195-3p | 1 | 0 | hsa-miR-505-3p | 1 | 1 |
| hsa-miR-195-5p | 1 | 1 | hsa-miR-532-3p | 1 | 0 |
| hsa-miR-196a-3p | 1 | 0 | hsa-miR-532-5p | 1 | 1 |
| hsa-miR-196a-5p | 1 | 1 | hsa-miR-542-3p | 1 | 1 |
| hsa-miR-196b-5p | 1 | 1 | hsa-miR-543 | 1 | 1 |
| hsa-miR-197-3p | 1 | 1 | hsa-miR-548av-3p\|h | 1 | 0 |
| hsa-miR-199a3p\|hs | 1 | 1 | hsa-miR-548av-5p\|h | 1 | 1 |
| hsa-miR-199a-5p | 1 | 1 | hsa-miR-548e-3p | 1 | 0 |
| hsa-miR-199b-5p | 1 | 1 | hsa-miR-5481 | 1 | 0 |
| hsa-miR-19b-3p | 1 | 1 | hsa-miR-548n | 1 | 0 |
| hsa-miR-200b-3p | 1 | 1 | hsa-miR-549a-3p | 1 | 0 |
| hsa-miR-200c-3p | 1 | 0 | hsa-miR-549a-5p | 1 | 0 |
| hsa-miR-203a-3p | 1 | 0 | hsa-miR-570-3p | 1 | 0 |
| hsa-miR-204-5p | 1 | 1 | hsa-miR-574-3p | 1 | 1 |
| hsa-miR-20a-5p | 1 | 1 | hsa-miR-574-5p | 1 | 1 |
| hsa-miR-20b-5p | 1 | 0 | hsa-miR-576-3p | 1 | 1 |
| hsa-miR-210-3p | 1 | 0 | hsa-miR-576-5p | 1 | 0 |
| hsa-miR-2110 | 1 | 1 | hsa-miR-577 | 1 | 0 |
| hsa-miR-2116-3p | 1 | 1 | hsa-miR-584-5p | 1 | 0 |
| hsa-miR-212-5p | 1 | 1 | hsa-miR-585-3p | 1 | 0 |
| hsa-miR-21-3p | 1 | 1 | hsa-miR-589-5p | 1 | 1 |
| hsa-miR-214-3p | 1 | 1 | hsa-miR-605-3p | 1 | 0 |
| hsa-miR-214-5p | 1 | 0 | hsa-miR-6132 | 1 | 0 |
| hsa-miR-215-5p | 1 | 0 | hsa-miR-615-3p | 1 | 1 |
| hsa-miR-21-5p | 1 | 1 | hsa-miR-616-5p | 1 | 0 |
| hsa-miR-216a-5p | 1 | 0 | hsa-miR-618 | 1 | 0 |
| hsa-miR-218-5p | 1 | 1 | hsa-miR-625-3p | 1 | 1 |
| hsa-miR-219b-3p | 1 | 0 | hsa-miR-625-5p | 1 | 0 |
| hsa-miR-219b-5p | 1 | 0 | hsa-miR-628-3p | 1 | 0 |
| hsa-miR-221-3p | 1 | 1 | hsa-miR-628-5p | 1 | 0 |
| hsa-miR-221-5p | 1 | 1 | hsa-miR-629-5p | 1 | 1 |
| hsa-miR-222-3p | 1 | 1 | hsa-miR-641 | 1 | 0 |
| hsa-miR-222-5p | 1 | 1 | hsa-miR-642a-5p | 1 | 0 |
| hsa-miR-22-3p | 1 | 1 | hsa-miR-6511b-3p | 1 | 0 |
| hsa-miR-224-5p | 1 | 1 | hsa-miR-651-5p | 1 | 0 |
| hsa-miR-22-5p | 1 | 1 | hsa-miR-652-3p | 1 | 0 |
| hsa-miR-2355-5p | 1 | 0 | hsa-miR-654-3p | 1 | 1 |
| hsa-miR-23a-3p | 1 | 1 | hsa-miR-654-5p | 1 | 1 |
| hsa-miR-23a-5p | 1 | 0 | hsa-miR-656-3p | 1 | 1 |
| hsa-mir-23b | 1 | 1 | hsa-miR-659-5p | 1 | 0 |
| hsa-miR-23b-3p | 1 | 1 | hsa-miR-660-5p | 1 | 1 |
| hsa-miR-23b-5p | 1 | 0 | hsa-miR-664a-3p | 1 | 0 |
| hsa-miR-23c | 1 | 1 | hsa-miR-664a-5p | 1 | 1 |
| hsa-miR-24-2-5p | 1 | 0 | hsa-miR-665 | 1 | 1 |
| hsa-miR-24-3p | 1 | 1 | hsa-miR-671-3p | 1 | 1 |
| hsa-mR-25-3p | 1 | 1 | hsa-miR-6716-3p | 1 | 0 |
| hsa-miR-25-5p | 1 | 0 | hsa-miR-6744-5p | 1 | 0 |
| hsa-miR-2682-5p | 1 | 0 | hsa-miR-6747-3p | 1 | 0 |
| hsa-miR-26a-5p | 1 | 1 | hsa-miR-6761-3p | 1 | 0 |
| hsa-miR-26b-3p | 1 | 0 | hsa-miR-6803-3p | 1 | 0 |
| hsa-miR-26b-5p | 1 | 1 | hsa-miR-6815-3p | 1 | 0 |
| hsa-miR-27a-3p | 1 | 1 | hsa-miR-6816-3p | 1 | 1 |
| hsa-miR-27a-5p | 1 | 1 | hsa-miR-6839-5p | 1 | 0 |
| hsa-miR-27b-3p | 1 | 1 | hsa-miR-6852-5p | 1 | 0 |
| hsa-miR-27b-5p | 1 | 0 | hsa-miR-6853-3p | 1 | 0 |
| hsa-miR-28-3p | 1 | 1 | hsa-miR-708-3p | 1 | 0 |
| hsa-miR-28-5p | 1 | 1 | hsa-miR-708-5p | 1 | 1 |
| hsa-miR-296-3p | 1 | 0 | hsa-miR-744-5p | 1 | 1 |
| hsa-miR-29a-3p | 1 | 1 | hsa-miR-758-3p | 1 | 1 |
| hsa-miR-29b-1-5p | 1 | 0 | hsa-miR-7-5p | 1 | 1 |
| hsa-miR-29b-3p | 1 | 0 | hsa-miR-769-5p | 1 | 1 |
| hsa-miR-29c-3p | 1 | 1 | hsa-miR-7706 | 1 | 1 |
| hsa-miR-3074-5p | 1 | 1 | hsa-miR-7976 | 1 | 0 |
| hsa-miR-30a-3p | 1 | 1 | hsa-miR-7977 | 1 | 0 |
| hsa-miR-30a-5p | 1 | 1 | hsa-miR-874-3p | 1 | 0 |
| hsa-miR-30b-5p | 1 | 1 | hsa-miR-887-5p | 1 | 0 |
| hsa-miR-30c-1-3p | 1 | 0 | hsa-miR-889-3p | 1 | 1 |
| hsa-miR-30c-2-3p | 1 | 0 | hsa-miR-891a-5p | 1 | 0 |
| hsa-miR-30c-5p | 1 | 1 | hsa-miR-92a-1-5p | 1 | 1 |
| hsa-miR-30d-3p | 1 | 0 | hsa-miR-92a-3p | 1 | 1 |
| hsa-miR-30d-5p | 1 | 1 | hsa-miR-92b-3p | 1 | 1 |
| hsa-miR-30e-3p | 1 | 1 | hsa-miR-92b-5p | 1 | 0 |
| hsa-miR-30e-5p | 1 | 1 | hsa-miR-935 | 1 | 0 |
| hsa-miR-3127-5p | 1 | 0 | hsa-miR-93-5p | 1 | 1 |
| hsa-miR-3129-5p | 1 | 1 | hsa-miR-941 | 1 | 1 |
| hsa-miR-3149 | 1 | 0 | hsa-miR-942-5p | 1 | 0 |
| hsa-miR-3158-3p | 1 | 0 | hsa-miR-95-3p | 1 | 0 |
| hsa-miR-31-5p | 1 | 1 | hsa-miR-98-3p | 1 | 0 |
| hsa-miR-3173-5p | 1 | 0 | hsa-miR-98-5p | 1 | 1 |
| hsa-miR-3180-5p | 1 | 0 | hsa-miR-9903 | 1 | 0 |
| hsa-miR-3190-3p | 1 | 0 | hsa-miR-9985 | 1 | 0 |
| hsa-miR-320a-3p | 1 | 1 | hsa-miR-99a-5p | 1 | 1 |
| hsa-miR-320b | 1 | 1 | hsa-miR-99b-3p | 1 | 1 |
| | | | hsa-miR-99b-5p | 1 | 1 |

The most abundant miRNAs identified in the tuned ASC-derived exosomes, ranked by read count, are presented in Table 7 below.

TABLE 7
Top miRNAs Identified in Tuned ASC-derived Exosomes

| miRNA | Read Count | Sequence | SEQ ID NO: |
|---|---|---|---|
| hsa-miR-100-5p | 178519 | AACCCGUAGAUCCGAACUUGUG | 126 |
| hsa-miR-10b-5p | 35809 | UACCCUGUAGAACCGAAUUUGUG | 127 |
| hsa-miR-143-3p | 26745 | UGAGAUGAAGCACUGUAGCUC | 128 |
| hsa-miR-10a-5p | 24235 | UACCCUGUAGAUCCGAAUUUGUG | 129 |
| hsa-miR-22-3p | 23499 | AAGCUGCCAGUUGAAGAACUGU | 130 |
| hsa-miR-125b-5p | 17449 | UCCCUGAGACCCUAACUUGUGA | 131 |
| hsa-miR-21-5p | 13780 | UAGCUUAUCAGACUGAUGUUGA | 132 |
| hsa-miR-99b-5p | 9939 | CACCCGUAGAACCGACCUUGCG | 133 |
| hsa-miR-125a-5p | 9802 | UCCCUGAGACCCUUUAACCUGUGA | 134 |
| hsa-let-7i-5p | 7900 | UGAGGUAGUAGUUUGUGCUGUU | 135 |
| hsa-miR-146b-5p | 6143 | UGAGAACUGAAUUCCAUAGGCUG | 136 |
| hsa-miR-99a-5p | 6038 | AACCCGUAGAUCCGAUCUUGUG | 137 |
| hsa-let-7f-5p | 3685 | UGAGGUAGUAGAUUGUAUAGUU | 138 |
| hsa-miR-24-3p | 3632 | UGGCUCAGUUCAGCAGGAACAG | 139 |
| hsa-let-7a-5p | 3346.6 | UGAGGUAGUAGGUUGUAUAGUU | 140 |
| hsa-miR-26a-5p | 3122 | UUCAAGUAAUCCAGGAUAGGCU | 141 |
| hsa-miR-148a-3p | 2810 | UCAGUGCACUACAGAACUUUGU | 142 |
| hsa-miR-1246 | 2660 | AAUGGAUUUUUGGAGCAGG | 143 |
| hsa-miR-199b-3p | 2516.5 | ACAGUAGUCUGCACAUUGGUUA | 144 |
| hsa-miR-199a-3p | 2516.5 | ACAGUAGUCUGCACAUUGGUUA | 145 |
| hsa-miR-199a-5p | 2218.5 | CCCAGUGUUCAGACUACCUGUUC | 146 |
| hsa-miR-23a-3p | 1737.5 | AUCACAUUGCCAGGGAUUUCC | 147 |
| hsa-miR-92a-3p | 1670 | UAUUGCACUUGUCCCGGCCUGU | 148 |
| hsa-miR-127-3p | 1352 | UCGGAUCCGUCUGAGCUUGGCU | 149 |
| hsa-miR-152-3p | 1277 | UCAGUGCAUGACAGAACUUGG | 150 |
| hsa-miR-146a-5p | 1268 | UGAGAACUGAAUUCCAUGGGUU | 151 |
| hsa-let-7g-5p | 1199 | UGAGGUAGUAGUUUGUACAGUU | 152 |
| hsa-miR-191-5p | 1192 | CAACGGAAUCCCAAAAGCAGCUG | 153 |
| hsa-miR-29a-3p | 1166 | UAGCACCAUCUGAAAUCGGUUA | 154 |
| hsa-miR-151a-3p | 977 | CUAGACUGAAGCUCCUUGAGG | 155 |
| hsa-let-7b-5p | 878 | UGAGGUAGUAGGUUGUGUGGUU | 156 |
| hsa-miR-199b-5p | 869.5 | CCCAGUGUUUAGACUAUCUGUUC | 157 |
| hsa-miR-145-3p | 862 | GGAUUCCUGGAAAUACUGUUCU | 158 |
| hsa-miR-221-3p | 840 | AGCUACAUUGUCUGCUGGGUUUC | 159 |
| hsa-miR-222-3p | 827 | AGCUACAUCUGGCUACUGGGU | 160 |
| hsa-miR-27b-3p | 716 | UUCACAGUGGCUAAGUUCUGC | 161 |
| hsa-miR-30d-5p | 714 | UGUAAACAUCCCCGACUGGAAG | 162 |
| hsa-miR-25-3p | 713 | CAUUGCACUUGUCUCGGUCUGA | 163 |
| hsa-miR-101-3p | 681 | UACAGUACUGUGAUAACUGAA | 164 |
| hsa-miR-221-5p | 675 | ACCUGGCAUACAAUGUAGAUUU | 165 |
| hsa-miR-423-3p | 602 | AGCUCGGUCUGAGGCCCCUCAGU | 166 |
| hsa-miR-186-5p | 567 | CAAAGAAUUCUCCUUUUGGGCU | 167 |
| hsa-miR-30a-5p | 505 | UGUAAACAUCCUCGACUGGAAG | 168 |
| hsa-miR-151a-5p | 503.5 | UCGAGGAGCUCACAGUCUAGU | 169 |
| hsa-miR-151b | 462.5 | UCGAGGAGCUCACAGUCU | 170 |
| hsa-miR-27a-3p | 451 | UUCACAGUGGCUAAGUUCCGC | 171 |
| hsa-miR-224-5p | 409 | UCAAGUCACUAGUGGUUCCGUUUAG | 172 |
| hsa-miR-181a-5p | 409 | AACAUUCAACGCUGUCGGUGAGU | 173 |
| hsa-miR-28-3p | 402 | CACUAGAUUGUGAGCUCCUGGA | 174 |
| hsa-miR-320a | 338.5 | GCCUUCUCUUCCCGGUUCUUCC | 175 |
| hsa-miR-378a-3p | 289 | ACUGGACUUGGAGUCAGAAGGC | 176 |
| hsa-miR-155-5p | 264 | UUAAUGCUAAUCGUGAUAGGGGUU | 177 |
| hsa-miR-193a-5p | 222 | UGGGUCUUUGCGGGCGAGAUGA | 178 |
| hsa-let-7c-5p | 220.16 | UGAGGUAGUAGGUUGUAUGGUU | 179 |
| hsa-miR-21-3p | 217 | CAACACCAGUCGAUGGGCUGU | 180 |
| hsa-miR-196a-5p | 202 | UAGGUAGUUUCAUGUUGUUGGG | 181 |
| hsa-miR-148b-3p | 187 | UCAGUGCAUCACAGAACUUUGU | 182 |
| hsa-miR-409-5p | 179 | AGGUUACCCGAGCAACUUUGCAU | 183 |
| hsa-miR-92b-3p | 174 | UAUUGCACUCGUCCCGGCCUCC | 184 |
| hsa-miR-140-3p | 169 | UACCACAGGGUAGAACCACGG | 185 |
| hsa-miR-486-5p | 158 | UCCUGUACUGAGCUGCCCCGAG | 186 |
| hsa-miR-30e-5p | 148 | UGUAAACAUCCUUGACUGGAAG | 187 |
| hsa-miR-615-3p | 146 | UCCGAGCCUGGGUCUCCCUCUU | 188 |
| hsa-miR-409-3p | 140 | GAAUGUUGCUCGGUGAACCCCU | 189 |
| hsa-miR-501-3p | 134.5 | AAUGCACCCGGGCAAGGAUUCU | 190 |
| hsa-miR-192-5p | 121.5 | CUGACCUAUGAAUUGACAGCC | 191 |
| hsa-miR-103a-3p | 115.5 | AGCAGCAUUGUACAGGGCUAUGA | 192 |
| hsa-miR-26b-5p | 115 | UUCAAGUAAUUCAGGAUAGGU | 193 |
| hsa-miR-1271-5p | 114 | CUUGGCACCUAGCAAGCACUCA | 194 |
| hsa-miR-500a-3p | 113.5 | AUGCACCUGGGCAAGGAUUCUG | 195 |
| hsa-miR-145-5p | 107 | GUCCAGUUUUCCCAGGAAUCCCU | 196 |
| hsa-miR-484 | 106 | UCAGGCUCAGUCCCCUCCCGAU | 197 |
| hsa-miR-125b-2-3p | 105 | UCACAAGUCAGGCUCUUGGGAC | 198 |
| hsa-miR-361-5p | 103 | UUAUCAGAAUCUCCAGGGGUAC | 199 |
| hsa-miR-503-5p | 102 | UAGCAGCGGGAACAGUUCUGCAG | 200 |
| hsa-miR-379-5p | 101 | UGGUAGACUAUGGAACGUAGG | 201 |

The identified miRNAs were categorized according to functional relevance in skin health applications. Table 8 below presents miRNAs associated with collagen production.

TABLE 8
miRNAs Associated with Collagen Production

| miRNA | Significance |
|---|---|
| miR-143-3p | Inhibits myofibroblast formation and collagen synthesis in |
| | cases of dermal fibrosis |
| miR-29a-3p | Anti-fibrotic miRNA that reduces collagen expression by |
| | binding to the 3′ UTR of collagen mRNA |
| miR-21-5p | Promotes fibrosis and wound healing, increasing collagen |
| | deposition |
| miR-140-3p | Protects cartilage by enhancing COL2A1 synthesis |
| miR-25-3p | Inhibits collagen production |
| miR-21-3p | Promotes fibrosis and wound healing, increasing collagen |
| | deposition |

Table 9 below presents miRNAs associated with skin barrier function.

TABLE 9
miRNAs Associated with Skin Barrier Function

| miRNA | Significance |
|---|---|
| miR-143 | Protective regulator against keratinocyte damage |
| miR-155-5p | Regulator of allergic inflammation |
| miR-146a | Negative regulator of inflammation |

Table 10 below presents miRNAs associated with immune regulation and anti-inflammatory activity.

TABLE 10
miRNAs Associated with Immune Regulation
and Anti-Inflammatory Activity

| miRNA | Significance |
|---|---|
| miR-143-3p | Suppressor of inflammation |
| miR-21 | Regulator of immune responses |
| let-7i-5p | Anti-inflammatory regulator |
| miR-199a-3p | Regulates macrophage polarization, autophagy, and |
| | apoptosis |
| miR-26a | Suppressor of excessive inflammation |
| miR-29a-3p | Regulates inflammation and T-cell function |
| miR-27b | Modulates inflammation and immune cell function |
| miR-155-5p | Regulator of the immune system |
| miR-26b | Suppressor of excessive inflammation |
| miR-10a-5p | Regulates macrophage polarization and modulates T cell |
| | function |
| miR-99b | Suppressor of inflammation |
| miR-146b | Suppressor of inflammation by inhibiting NF-κB signaling |
| miR-24 | Modulates T cell differentiation |
| miR-146a | Negative regulator of the immune system |
| miR-145 | Modulates inflammatory responses |
| miR-25 | Regulates inflammation |
| miR-140-3p | Immune regulator |

Table 11 below presents miRNAs associated with skin cell proliferation.

TABLE 11
miRNAs Associated with Cell Proliferation

| miRNA | Significance |
|---|---|
| miR-100-5p | targets MTMR3 to activate the PIP3/AKT and ERK |
| | signaling pathways |
| miR-125-5p | Inhibits excessive proliferation of skin cells |
| miR-24-3p | Inhibits proliferation |
| miR-146a-5p | Promotes proliferation |
| miR-222-3p | Enhances proliferation, migration, and angiogenesis |
| miR-22-3p | Promotes proliferation by activating Wnt signaling |
| miR-21-5p | Promotes proliferation |
| let-7a-5p | Inhibits proliferation |
| miR-29a-3p | Inhibits cell cycle progression |

Table 12 below presents miRNAs associated with wound healing.

TABLE 12
miRNAs Associated with Wound Healing

| miRNA | Significance |
|---|---|
| miR-10b | Promotes cell proliferation, migration, and epithelial- |
| | mesenchymal transition |
| miR-21 | Promotes keratinocyte migration, fibroblast proliferation, and |
| | collagen contraction |
| miR-146a | Anti-inflammatory and promotes tissue repair |
| miR-10a | Regulates proliferation and migration |
| miR-146b | Reduces excessive inflammation and suppresses scar tissue |
| | formation |
| miR-155 | Inflammation regulator |

Table 13 below presents miRNAs associated with angiogenesis.

TABLE 13
miRNAs Associated with Angiogenesis

| miRNA | Significance |
|---|---|
| miR-143 | Angiogenic inhibitor |
| miR-125-5p | Promotes angiogenesis during wound healing |
| miR-125a-5p | Promotes angiogenesis during wound healing |
| miR-26a-5p | Regulates angiogenesis by targeting endothelial cell |
| | signaling pathways |
| miR-29a-3p | Regulates angiogenesis by targeting endothelial cell |
| | signaling pathways |
| miR-222 | Regulates angiogenesis by targeting endothelial cell |
| | signaling pathways and vascular formation |
| miR-186-5p | Angiogenic inhibitor |
| miR-155-5p | Regulates angiogenesis by targeting endothelial cell |
| | signaling pathways |
| miR-22-3p | Regulates angiogenesis by targeting endothelial cell |
| | signaling pathways |
| miR-21-5p | Regulates VEGF signaling |
| miR-199a-3p | Regulates VEGF signaling |
| miR-92a-3p | Regulates endothelial cell proliferation, migration, and tube |
| | formation |
| miR-221 | Modulates endothelial cell function |
| miR-27b | Promotes vascular sprouting |
| miR-181a-5p | Regulates angiogenesis by targeting key signaling pathways |
| let-7b | Modulates endothelial cell function |

Table 14 below presents miRNAs associated with melanogenesis and pigmentation, along with the functional significance of each miRNA.

TABLE 14
miRNAs Associated with Melanogenesis and Pigmentation

| miRNA | Significance |
|---|---|
| miR-125 | Reduces melanin content by inhibiting pigment expressing |
| | genes TYR, TYRP1, DCT, SH3BP4 |
| miR-21-5p | Positively regulates melanogenesis SOX5 |
| miR-148a-3p | Targets MITF |
| miR-143-5p | Decreases melanin Myo5a, TAK1 |
| miR-221-3p | Reduces melanin formation TYRP1 |
| miR-222 | Regulates melanogenesis |
| miR-25 | Decreases melanogenesis via MITF |
| miR-27a-3p | Reduces melanin content in epidermal cells, Wnt3a |
| miR-21a-3p | Regulates |

Table 15 below presents miRNAs associated with photoaging and UV damage protection, along with the functional significance of each miRNA.

TABLE 15
miRNAs Associated with Photoaging and UV Damage Protection

| miRNA | Significance |
|---|---|
| miR-125b | Prohibits prolonged hyperactivation of p38α by UV radiation |
| | which is required for protecting cells from UV induced |
| | apoptosis |
| miR-21-5p | Regulates healthy aging phenotype in skin |
| miR-23a | Regulates DNA repair after UV damage |
| miR-146a | Reduces damage due to photoaging |
| miR-145 | Alleviates IL-6 induced increase of sensitivity to UVB |
| | irradiation |
| miR-155 | Protects dermal fibroblasts via control of c-Jun expression; |
| | recognized as a potential treatment for photoaging |
| miR-21-3p | Reduces UV induced inflammation |

Table 16 below presents miRNAs associated with acne, along with the functional significance of each miRNA.

TABLE 16
miRNAs Associated with Acne

| miRNA | Significance |
|---|---|
| miR-143 | Reduces acne induced inflammation; reduces TLR2 protein |
| miR-146a-5p | Decreases IL-8 in acne lesions; suppresses Th1 type responses in skin |

Table 17 below presents miRNAs identified in the tuned ASC-derived exosomes that are associated with hair growth and inhibition of hair loss, along with the functional significance of each miRNA.

TABLE 17
miRNAs Associated with Hair Growth and Inhibition of Hair Loss

| miRNA | Significance |
|---|---|
| miR-21-5p | Regulates Hair Follicle development CNKSR2 KLF3 TNPO1 |
| miR-24 | Increases Hair Keratinocyte differentiation |
| miR-26a-5p | Regulates Hair Follicle development and growth TGF/B-catenin |
| miR-146a | Targets and inhibits DKK1 |
| let-7b | Regulates hair growth via targeting EDA |
| miR-101-3p | Promotes proliferation and inhibits apoptosis of hair follicle stem cells by |
| | targeting DUSP1; Also targets and inhibits DKK1 |
| miR-181a-5p | Promotes Hair Growth |
| miR-21-3p | Regulates Hair Follicle development CNKSR2 KLF3 TNPO1 |
| miR-148b-3p | Promotes proliferation of Hair Follicle stem cells NFAT5 |
| miR-481-5p | Maintains anagen phase; prevents immune privilege collapse of follicles in |
| | Alopecia Areata |
| miR-103a-3p | Targets and decreases DKK1 |

### Gene Expression Analysis

### Project Summary

This study, as previously described in U.S. Non-Provisional application Ser. No. 19/270,704, was conducted to evaluate how tuned ASC-derived exosomes and tuned Aloe-derived exosomes influence gene expression in human follicle dermal papilla cells (HFDPCs). HFDPCs are mesenchymal-derived cells at the base of the hair follicle that regulate hair follicle growth through the expression and secretion of specific biomarkers.

Gene expression was assessed using a qPCR-based gene expression panel containing 52 target genes (see, Table 18) at 24-hour and 72-hour time points following application of the test materials to dermal papillae cells with varying dose concentrations to evaluate the effects of exosome source, tuning status, dose, and exposure duration on gene regulation.

TABLE 18
Dermal Papilla Cell Panel Gene Information

| Gene ID | Gene Name | Associated Function(s) |
|---|---|---|
| AR | Androgen Receptor | Hormone Regulation |
| ASIP | Agouti Signaling Protein | Pigmentation |
| AXIN2 | Axin 2 | Wnt/BCT Pathway |
| BAX | BCL2 Associated X, Apoptosis Regulator | Proliferation, Cell Cycle, and Apoptosis |
| BCL2 | BCL2 Apoptosis Regulator | Proliferation, Cell Cycle, and Apoptosis |
| BMP2 | Bone Morphogenic Protein 2 | BMP Pathway |
| BMP4 | Bone Morphogenic Protein 4 | BMP Pathway |
| BMP6 | Bone Morphogenic Protein 6 | BMP Pathway |
| CCND1 | Cyclin D1 | Proliferation, Cell Cycle, and Apoptosis |
| CDKN2A | Cyclin Dependent Kinase Inhibitor 2A | Proliferation, Cell Cycle, and Apoptosis |
| CORIN | Corin, Serine Peptidase | Anagen Marker/Inducer |
| CTNNB1 | Catenin Beta 1 | Wnt/BCT Pathway |
| DCN | Decorin | Anagen Marker/Inducer |
| DKK1 | Dickkopf WNT Signaling Pathway Inhibitor 1 | Wnt/BCT Pathway |
| ESR1 | Estrogen Receptor 1 | Hormone Regulation |
| ESR2 | Estrogen Receptor 2 | Hormone Regulation |
| FGF10 | Fibroblast Growth Factor 10 | Anagen Marker/Inducer |
| FGF5 | Fibroblast Growth Factor 5 | Catagen Marker/Inducer |
| FGF7 | Fibroblast Growth Factor 7 | Anagen Marker/Inducer |
| GLI1 | GLI Family Zinc Finger 1 | Hair Follicle Regrowth |
| GSK3B | Glycogen Synthase Kinase 3 Beta | Wnt/BCT Pathway |
| HES | Hes Family BHLH Transcription Factor 1 | Notch Signaling Pathway |
| HEY1 | Hes Related Family BHLH Transcription | Anagen Marker/Inducer |
| | Factor with YRPW Motif 1 | |
| HGF | Hepatocyte Growth Factor | Proliferation, Cell Cycle, and Apoptosis |
| IGF1 | Insulin Like Growth Factor 1 | Proliferation, Cell Cycle, and Apoptosis |
| IL1B | Interleukin 1 Beta | Catagen Marker/Inducer |
| IL6 | Interleukin 6 | Catagen Marker/Inducer |
| KITLG | KIT Ligand | Pigmentation |
| LEF1 | Lymphoid Enhancer Binding Factor 1 | Wnt/BCT Pathway |
| LEP | Leptin | Anagen Marker/Inducer |
| MC1R | Melanocortin 1 Receptor | Pigmentation |
| MKI67 | Marker of Proliferation Ki-67 | Proliferation, Cell Cycle, and Apoptosis |
| NOG | Noggin | BMP Pathway |
| NOTCH1 | Notch Receptor 1 | Notch Signaling Pathway |
| PDGFA | Platelet Derived Growth Factor Subunit A | Proliferation, Cell Cycle, and Apoptosis |
| PROM1 | Prominin 1 | Anagen Marker/Inducer |
| PTCH1 | Patched 1 | Hair Follicle Regrowth |
| SFRP1 | Secreted Frizzled Related Protein 1 | Wnt/BCT Pathway |
| SOX2 | SRY-Box Transcription Factor 2 | BMP Pathway |
| SRD5A2 | Steroid 5 Alpha-Reductase 2 | Hormone Regulation |
| STAT3 | Signal Transducer and Activator of | JAK-STAT Pathway |
| | Transcription 3 | |
| STAT5A | Signal Transducer and Activator of | JAK-STAT Pathway |
| | Transcription 5A | |
| STAT6 | Signal Transducer and Activator of | JAK-STAT Pathway |
| | Transcription 6 | |
| TCF4 | Transcription Factor 4 | Wnt/BCT Pathway |
| TGFB1 | Transforming Growth Factor Beta 1 | Wnt/BCT Pathway |
| TGFB2 | Transforming Growth Factor Beta 2 | Catagen Marker/Inducer |
| VCAN | Versican | Anagen Marker/Inducer |
| VEGFA | Vascular Endothelial Growth Factor A | Proliferation, Cell Cycle, and Apoptosis |
| WIF1 | WNT Inhibitory Factor 1 | Wnt/BCT Pathway |
| WNT10B | Wnt Family Member 10B | Wnt/BCT Pathway |
| WNT3A | Wnt Family Member 3A | Wnt/BCT Pathway |
| WNTA | Wnt Family Member 5A | Wnt/BCT Pathway |

### Experimental Procedure

As described previously in U.S. Non-Provisional application Ser. No. 19/270,704, human follicle dermal papilla cells (HFDPCs) sourced from the temple skin of a 50-year-old Caucasian female donor were seeded into culture plates and treated with test materials comprising native and tuned (heat shocked) Aloe-derived exosomes and native and tuned (heat shocked) ASC-derived exosomes at varying concentrations, alongside untreated, positive (4% FBS media), and vehicle (PBS) controls.

Following exposure to the test materials for 1 hour, fresh maintenance media was added and the cells were incubated for 24 hours or 72 hours. Total RNA was then isolated and cDNA was synthesized. Gene expression was assessed using the Genemarkers Dermal Papilla Cell Panel, a qPCR-based gene expression panel containing 52 target genes and 4 endogenous control genes. Cytotoxicity was assessed via LDH assay to confirm that the test materials did not compromise cell viability.

For the LDH cytotoxicity assay, each sample of used culture media was diluted 1:10 with sterile phosphate-buffered saline (PBS). A background control (diluted culture media that was not used for cell culture), a “low control” (diluted treatment media collected from the untreated culture wells), and a “high control” (diluted culture media collected from the 1% Triton-X 100™ treated culture wells) were included in the assay. Each diluted sample was added to an optically clear, flat-bottom 96-well plate in duplicate.

The LDH reaction mixture (Roche®) was prepared and added to each aliquot of diluted media (1:1). The reaction plate was incubated for about 20 minutes at room temperature, protected from light. Stopping solution (1.0N HCl) was then added to each well and absorbance was measured at 492 nm with a reference filter at 620 nm. Each sample absorbance value was calculated as the mean OD492-OD620 value for the duplicate reaction wells, with the blank absorbance value subtracted.

The % cytotoxicity was then calculated relative to the Untreated Control (negative control, set to 0% cytotoxicity) and the Triton X-100™ treated control (positive control, set to 100% cytotoxicity) absorbance values, according to kit instructions:

% Cytotoxicity=[(Test Media Value−Low Control)/(High Control−Low Control)]*100.

qPCR data quality and statistical analysis were assessed and performed on the raw data files using ThermoFisher® Connect Software (Life Technologies®). Statistical analysis was performed using the relative quantitation (RQ) method. In the first step of an RQ analysis, the Cq value of the target gene is normalized to the Cq value of an endogenous control gene to generate the delta Cq (dCq). dCq values are calculated in order to normalize for variability between the samples that may occur during the experimental procedures.

### Results

Gene expression analysis demonstrated that heat shocked ASC-derived exosomes regulated the following genes in dermal papillae cells: BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, and WNT5A. Manufacturing methods affected ASC-derived exosome regulation of gene expression, with heat shocked ASC-derived exosomes demonstrating different gene expression regulation affecting different genes and different levels of regulation on the same gene (data not shown). Comparing gene expression analysis results at 24 hours and 72 hours showed that different genes were affected at different time points by the tuned ASC-derived exosomes (data not shown).

### Example 4: Clinical Evaluation of Topical Exosome Compositions for Facial Skin Improvement

### Rationale

A clinical study was conducted to compare the efficacy of two topical moisturizer formulations containing exosomes for improving facial skin appearance. The study evaluated wrinkle reduction, skin firmness, skin elasticity, skin luminosity, and skin topography in human subjects over a four-week treatment period.

### Materials and Methods

Twenty subjects were enrolled in the study and divided into two treatment groups.

Ten subjects were treated with a composition containing tuned (heat shocked) ASC-derived exosomes. ASC-derived exosomes were isolated from cells cultured at 37 °C and subjected to a 2 hour heat shock step at 43 °C.

Ten subjects were treated with a composition containing tuned (heat shocked) Aloe-derived exosomes. Aloe-derived exosomes were isolated after exposure to 35 °C for 2 hours followed by 45 °C for 3 hours, followed by 18-25 °C for 24 hours.

Subjects were instructed to apply the assigned composition topically to the face twice daily for four weeks. Dime to quarter size amounts were applied, sufficient to cover the entire face.

Skin parameters were assessed at baseline and at weeks 1, 2, and 4 using multiple evaluation methods.

Wrinkle reduction was assessed by expert grader evaluation of crow's feet on both the left and right sides of the face using a standardized grading scale where 0=None, 0.5=Barely perceptible, 1=Mild, 2=Moderate, 3=Marked, and 4=Severe.

Skin topography measurements including skin roughness (Ra values), wrinkle depth volume, and mean wrinkle depth were obtained using PRIMOS® 3D skin analysis. Ra (average roughness) measures the deviation of a surface from a mean height. PRIMOS® 3D skin analysis is a high-resolution, non-invasive imaging system that uses digital fringe projection to map skin surface topography. It can provide precise, 3D measurements of wrinkle depth, skin roughness, scars, and volume changes. The system projects a pattern of parallel lines onto the skin. A camera captures how these lines are distorted by skin surface variations, creating a high-resolution, three-dimensional heightmap.

Skin firmness and elasticity were measured using a Cutometer® device (Courage+Khazaka). A Cutometer® device is a non-invasive device used to measure skin elasticity and firmness by applying suction. It creates a vacuum to pull the skin into a probe aperture and uses an optical sensor to measure how much the skin moves (firmness) and how fast it returns to normal (elasticity).

Skin luminosity and overall appearance were assessed using VISIA CR® imaging (Canfield Scientific). VISIA CR® imaging is a high-resolution facial imaging system that captures standardized, multi-spectral images to analyze skin health. It uses several lighting modes to measure wrinkles, spots, pores, color variations, and bacteria.

Subject questionnaires were administered and subjects were asked to self-evaluate their subjective perceptions of product efficacy. Statistical analysis was performed using repeated measures ANOVA with Bonferroni correction for multiple comparisons. Shapiro-Wilk's W Test for Normality and Brown-Forsythe Equal Variance Test were performed to verify statistical assumptions. Between-groups analysis was conducted using Two-Way repeated measures ANOVA to compare the two treatment formulations.

### Results

### Wrinkle Reduction

Wrinkle reduction was assessed by expert grader evaluation of crow's feet on both the left and right sides of the face using a standardized grading scale where 0=None, 0.5=Barely perceptible, 1=Mild, 2=Moderate, 3=Marked, and 4=Severe.

For subjects treated with the heat-shocked (tuned) ASC-derived exosomes, expert grader evaluation of crow's feet on the left side of the face demonstrated a mean percent change from baseline of −20.6% at week 1, −41.2% at week 2, and −67.6% at week 4.

At week 4, 90% of subjects showed decreased crow's feet scores and 10% showed no change, with 0% showing increased values. Statistical analysis revealed a statistically significant decrease in expert evaluator scores for crow's feet after 2 weeks (p=0.001) and 4 weeks (p=0.001) of treatment compared to baseline (see, Table 19).

TABLE 19
Expert Grader Evaluation of Crow's Feet (Left) - ASC-Derived
Exosomes: Difference from Baseline

| | | | Difference | | Difference | | Difference |
|---|---|---|---|---|---|---|---|
| Subject # | Baseline | Week 1 | Week 1 | Week 2 | Week 2 | Week 4 | Week 4 |
| 2 | 2.0 | 2.0 | 0.0 | 1.0 | −1.0 | 0.5 | −1.5 |
| 5 | 2.0 | 2.0 | 0.0 | 2.0 | 0.0 | 1.0 | −1.0 |
| 6 | 2.0 | 2.0 | 0.0 | 1.0 | −1.0 | 1.0 | −1.0 |
| 9 | 2.0 | 2.0 | 0.0 | 1.0 | −1.0 | 0.5 | −1.5 |
| 11 | 1.0 | 1.0 | 0.0 | 1.0 | 0.0 | 1.0 | 0.0 |
| 14 | 2.0 | 0.5 | −1.5 | 0.5 | −1.5 | 0.0 | −2.0 |
| 16 | 2.0 | 2.0 | 0.0 | 2.0 | 0.0 | 1.0 | −1.0 |
| 18 | 1.0 | 0.5 | −0.5 | 0.5 | −0.5 | 0.0 | −1.0 |
| 21 | 1.0 | 0.5 | −0.5 | 0.0 | −1.0 | 0.0 | −1.0 |
| 22 | 2.0 | 1.0 | −1.0 | 1.0 | −1.0 | 0.5 | −1.5 |
| Mean | 1.7 | 1.4 | −0.4 | 1.0 | −0.7 | 0.6 | −1.2 |
| Median | 2.0 | 1.5 | 0.0 | 1.0 | −1.0 | 0.5 | −1.0 |
| Standard Deviation | 0.5 | 0.7 | 0.5 | 0.6 | 0.5 | 0.4 | 0.5 |
| Maximum | 2.0 | 2.0 | 0.0 | 2.0 | 0.0 | 1.0 | 0.0 |
| Minimum | 1.0 | 0.5 | −1.5 | 0.0 | −1.5 | 0.0 | −2.0 |
| Total # of Subjects | 10 | | | | | | |
| # of Increased | 0 | | | | | | |
| Values | | | | | | | |
| # of Decreased | — | 4 | | 7 | | 9 | |
| Values | | | | | | | |
| # of Unchanged | — | 6 | | 3 | | 1 | |
| Values | | | | | | | |

Evaluation Key: 0 = None; 0.5 = Barely Perceptible; 1 = Mild; 2 = Moderate; 3 = Marked; 4 = Severe.

For crow's feet on the right side of the face in the ASC-derived exosome group, the mean percent change from baseline was −26.5% at week 1, −4.2% at week 2, and −67.6 at week 4.

At week 4, 100% of subjects showed decreased crow's feet scores. Statistical analysis demonstrated a statistically significant decrease in expert evaluator scores at weeks 1 (p=0.013), 2 (p=0.001), and 4 (p=0.001) compared to baseline. Three subjects in the ASC-derived exosome group achieved 100% reduction of wrinkles (see, Table 20).

TABLE 20
Expert Grader Evaluation of Crow's Feet (Right) - ASC-Derived
Exosomes: Difference from Baseline

| | | | Difference | | Difference | | Difference |
|---|---|---|---|---|---|---|---|
| Subject # | Baseline | Week 1 | Week 1 | Week 2 | Week 2 | Week 4 | Week 4 |
| 2 | 2.0 | 2.0 | 0.0 | 1.0 | −1.0 | 0.5 | −1.5 |
| 5 | 2.0 | 2.0 | 0.0 | 2.0 | 0.0 | 1.0 | −1.0 |
| 6 | 2.0 | 2.0 | 0.0 | 1.0 | −1.0 | 1.0 | −1.0 |
| 9 | 1.0 | 1.0 | 0.0 | 1.0 | 0.0 | 0.5 | −0.5 |
| 11 | 2.0 | 1.0 | −1.0 | 1.0 | −1.0 | 1.0 | −1.0 |
| 14 | 2.0 | 0.5 | −1.5 | 0.5 | −1.5 | 0.0 | −2.0 |
| 16 | 2.0 | 2.0 | 0.0 | 2.0 | 0.0 | 1.0 | −1.0 |
| 18 | 1.0 | 0.5 | −0.5 | 0.5 | −0.5 | 0.0 | −1.0 |
| 21 | 1.0 | 0.5 | −0.5 | 0.0 | −1.0 | 0.0 | −1.0 |
| 22 | 2.0 | 1.0 | −1.0 | 1.0 | −1.0 | 0.5 | −1.5 |
| Mean | 1.7 | 1.3 | −0.5 | 1.0 | −0.7 | 0.6 | −1.2 |
| Median | 2.0 | 1.0 | −0.3 | 1.0 | −1.0 | 0.5 | −1.0 |
| Standard | 0.5 | 0.7 | 0.6 | 0.6 | 0.5 | 0.4 | 0.4 |
| Deviation | | | | | | | |
| Maximum | 2.0 | 2.0 | 0.0 | 2.0 | 0.0 | 1.0 | −0.5 |
| Minimum | 1.0 | 0.5 | −1.5 | 0.0 | −1.5 | 0.0 | −2.0 |
| Total # of | 10 | | | | | | |
| Subjects | | | | | | | |
| # of Increased | 0 | | | | | | |
| Values | | | | | | | |
| # of Decreased | — | 5 | | 7 | | 10 | |
| Values | | | | | | | |
| # of Unchanged | — | 5 | | 3 | | 0 | |
| Values | | | | | | | |

Evaluation Key: 0 = None; 0.5 = Barely Perceptible; 1 = Mild; 2 = Moderate; 3 = Marked; 4 = Severe.

For subjects treated with heat-shocked (tuned) Aloe-derived exosomes, expert grader evaluation of crow's feet on the left side of the face demonstrated a mean percent change from baseline of −23.3% at week 1, −33.3% at week 2, and −46.7% at week 4. Statistical analyses revealed a statistically significant decrease in expert evaluator scores at weeks 1 (p=0.038), 2 (p=0.002), and 4 (p=0.001) compared to baseline (see, Table 21).

TABLE 21
Expert Grader Evaluation of Crow's Feet (Left) - Aloe-Derived
Exosomes: Difference from Baseline

| | | | Difference | | Difference | | Difference |
|---|---|---|---|---|---|---|---|
| Subject # | Baseline | Week 1 | Week 1 | Week 2 | Week 2 | Week 4 | Week 4 |
| 1 | 2.0 | 2.0 | 0.0 | 2.0 | 0.0 | 2.0 | 0.0 |
| 3 | 1.0 | 1.0 | 0.0 | 1.0 | 0.0 | 0.5 | −0.5 |
| 7 | 1.0 | 1.0 | 0.0 | 0.5 | −0.5 | 0.5 | −0.5 |
| 8 | 1.0 | 1.0 | 0.0 | 1.0 | 0.0 | 1.0 | 0.0 |
| 10 | 2.0 | 2.0 | 0.0 | 1.0 | −1.0 | 1.0 | −1.0 |
| 12 | 2.0 | 1.0 | −1.0 | 1.0 | −1.0 | 0.5 | −1.5 |
| 13 | 2.0 | 1.0 | −1.0 | 1.0 | −1.0 | 1.0 | −1.0 |
| 15 | 2.0 | 1.0 | −1.0 | 1.0 | −1.0 | 0.5 | −1.5 |
| 17 | 1.0 | 0.5 | −0.5 | 0.5 | −0.5 | 0.5 | −0.5 |
| 20 | 1.0 | 1.0 | 0.0 | 1.0 | 0.0 | 0.5 | −0.5 |
| Mean | 1.5 | 1.2 | −0.4 | 1.0 | −0.5 | 0.8 | −0.7 |
| Median | 1.5 | 1.0 | 0.0 | 1.0 | −0.5 | 0.5 | −0.5 |
| Standard | 0.5 | 0.5 | 0.5 | 0.4 | 0.5 | 0.5 | 0.5 |
| Deviation | | | | | | | |
| Maximum | 2.0 | 2.0 | 0.0 | 2.0 | 0.0 | 2.0 | 0.0 |
| Minimum | 1.0 | 0.5 | −1.0 | 0.5 | −1.0 | 0.5 | −1.5 |
| Total # of | 10 | | | | | | |
| Subjects | | | | | | | |
| # of Increased | 0 | | | | | | |
| Values | | | | | | | |
| # of Decreased | — | 4 | | 6 | | 8 | |
| Values | | | | | | | |
| # of Unchanged | — | 6 | | 4 | | 2 | |
| Values | | | | | | | |

Evaluation Key: 0 = None; 0.5 = Barely Perceptible; 1 = Mild; 2 = Moderate; 3 = Marked; 4 = Severe.

For crow's feet on the right side of the face in the Aloe-derived exosome group, the mean percent change from baseline was −26.7% at week 1, −43.3% at week 2, and −53.3% at week 4. At week 4, 90% of subjects showed decreased values and 10% showed no change. Statistical analysis demonstrated a statistically significant decrease in expert evaluator scores at weeks 1 (p=0.014), 2 (p=0.001), and 4 (p=0.001) compared to baseline (see, Table 22).

TABLE 22
Expert Grader Evaluation of Crow's Feet (Right) - Aloe-Derived
Exosomes: Difference from Baseline

| | | | Difference | | Difference | | Difference |
|---|---|---|---|---|---|---|---|
| Subject # | Baseline | Week 1 | Week 1 | Week 2 | Week 2 | Week 4 | Week 4 |
| 1 | 2.0 | 2.0 | 0.0 | 1.0 | −1.0 | 1.0 | −1.0 |
| 3 | 1.0 | 1.0 | 0.0 | 1.0 | 0.0 | 0.5 | −0.5 |
| 7 | 1.0 | 1.0 | 0.0 | 0.5 | −0.5 | 0.5 | −0.5 |
| 8 | 1.0 | 1.0 | 0.0 | 1.0 | 0.0 | 1.0 | 0.0 |
| 10 | 2.0 | 2.0 | 0.0 | 1.0 | −1.0 | 1.0 | −1.0 |
| 12 | 2.0 | 1.0 | −1.0 | 1.0 | −1.0 | 0.5 | −1.5 |
| 13 | 2.0 | 1.0 | −1.0 | 1.0 | −1.0 | 1.0 | −1.0 |
| 15 | 2.0 | 1.0 | −1.0 | 1.0 | −1.0 | 0.5 | −1.5 |
| 17 | 1.0 | 0.5 | −0.5 | 0.5 | −0.5 | 0.5 | −0.5 |
| 20 | 1.0 | 0.5 | 0.0 | 0.5 | −0.5 | 0.5 | −0.5 |
| Mean | 1.5 | 1.1 | −0.4 | 0.9 | −0.7 | 0.7 | −0.8 |
| Median | 1.5 | 1.0 | −0.3 | 1.0 | −0.8 | 0.5 | −0.8 |
| Standard | 0.5 | 0.5 | 0.5 | 0.2 | 0.4 | 0.3 | 0.5 |
| Deviation | | | | | | | |
| Maximum | 2.0 | 2.0 | 0.0 | 1.0 | 0.0 | 1.0 | 0.0 |
| Minimum | 1.0 | 0.5 | −1.0 | 0.5 | −1.0 | 0.5 | −1.5 |
| Total # of | 10 | | | | | | |
| Subjects | | | | | | | |
| # of Increased | 0 | | | | | | |
| Values | | | | | | | |
| # of Decreased | — | 5 | | 8 | | 9 | |
| Values | | | | | | | |
| # of Unchanged | — | 5 | | 2 | | 1 | |
| Values | | | | | | | |

Evaluation Key: 0 = None; 0.5 = Barely Perceptible; 1 = Mild; 2 = Moderate; 3 = Marked; 4 = Severe.

### PRIMOS® 3D Skin Topography

Skin roughness was assessed using Ra values (μm), where a decrease in Ra value represents a decrease in surface roughness (smoother skin). For the heat-shocked ASC-derived exosome group, the mean Ra value was 17.7 μm at baseline, 17.2 μm at week 1, 17.7 μm at week 2, and 17.7 μm at week 4 (see, Table 23).

The mean percent change from baseline was −2.9% at week 1, −0.2%0 at week 2, and −0.2% at week 4. At week 4, 6000 of subjects showed decreased Ra values and 400% showed increased values (see, Table 23).

TABLE 23
Skin Roughness Ra Values - ASC-Derived Exosomes: Difference from Baseline

| | | | Difference | | Difference | | Difference |
|---|---|---|---|---|---|---|---|
| Subject # | Baseline | Week 1 | Week 1 | Week 2 | Week 2 | Week 4 | Week 4 |
| 2 | 13.3 | 14.5 | 1.2 | 12.5 | −0.8 | 12.9 | −0.4 |
| 5 | 22.4 | 19.6 | −2.8 | 24.6 | 2.2 | 24.2 | 1.8 |
| 6 | 13.8 | 14.0 | .2 | 13.7 | −0.1 | 13.9 | 0.1 |
| 9 | 16.3 | 15.5 | −0.8 | 16.2 | −0.1 | 16.0 | −0.3 |
| 11 | 17.5 | 15.8 | −1.7 | 17.3 | −0.2 | 16.1 | −1.4 |
| 14 | 23.2 | 20.4 | −2.8 | 21.1 | −2.1 | 21.7 | −1.5 |
| 16 | 15.6 | 14.6 | −1.0 | 15.0 | −0.6 | 14.7 | −0.9 |
| 18 | 21.7 | 21.4 | −0.3 | 23.3 | 1.6 | 21.6 | −0.1 |
| 21 | 15.6 | 17.7 | 2.1 | 16.2 | 0.6 | 16.7 | 1.1 |
| 22 | 17.7 | 18.4 | 0.7 | 16.8 | −0.9 | 19.0 | 1.3 |
| Mean | 17.7 | 17.2 | −0.5 | 17.7 | 0.0 | 17.7 | 0.0 |
| Median | 16.9 | 16.8 | −0.6 | 16.5 | −0.2 | 16.4 | −0.2 |
| Standard | 3.6 | 2.7 | 1.6 | 4.0 | 1.2 | 3.8 | 1.1 |
| Deviation | | | | | | | |
| Maximum | 23.2 | 21.4 | 2.1 | 24.6 | 2.2 | 24.2 | 1.8 |
| Minimum | 13.3 | 14.0 | −2.8 | 12.5 | −2.1 | 12.9 | −1.5 |
| Total # of | 10 | | | | | | |
| Subjects | | | | | | | |
| # of Increased | — | 4 | | 3 | | 4 | |
| Values | | | | | | | |
| # of Decreased | — | 6 | | 7 | | 6 | |
| Values | | | | | | | |
| # of Unchanged | — | 0 | | | | | |
| Values | | | | | | | |

Skin roughness was assessed using Ra values (μm), where a decrease in Ra value represents a decrease in surface roughness (smoother skin). For the heat-shocked Aloe-derived exosome group, the mean Ra value was 19.1 μm at baseline, 19.7 μm at week 1, 19.7 μm at week 2, and 18.6 μm at week 4 (see, Table 24).

The mean percent change from baseline was 3.3% at week 1, 3.4% at week 2, and −2.3% at week 4. At week 4, 40% of subjects showed decreased Ra values and 60% showed increased values (see, Table 24).

TABLE 24
Skin Roughness Ra Values - Aloe-Derived Exosomes: Difference from Baseline

| | | | Difference | | Difference | | Difference |
|---|---|---|---|---|---|---|---|
| Subject # | Baseline | Week 1 | Week 1 | Week 2 | Week 2 | Week 4 | Week 4 |
| 1 | 19.5 | 19.2 | −0.3 | 21.8 | 2.3 | 20.8 | 1.3 |
| 3 | 15.1 | 15.2 | 0.1 | 15.1 | 0.0 | 15.9 | 0.8 |
| 7 | 16.3 | 15.3 | −1.0 | 16.1 | −0.2 | 16.4 | 0.1 |
| 8 | 23.6 | 21.7 | −1.9 | 19.8 | −3.8 | 18.4 | −5.2 |
| 10 | 16.6 | 16.6 | 0.0 | 18.7 | 2.1 | 16.7 | 0.1 |
| 12 | 16.3 | 16.8 | 0.5 | 18.8 | 2.5 | 18.4 | 2.1 |
| 13 | 28.9 | 21.3 | −7.6 | 28.1 | −0.8 | 22.8 | −6.1 |
| 15 | 14.5 | 14.0 | −0.5 | 12.9 | −1.6 | 13.2 | −1.3 |
| 17 | 18.5 | 18.3 | −0.2 | 14.7 | −3.8 | 14.9 | −3.6 |
| 20 | 21.3 | 38.4 | 17.1 | 31.0 | 9.7 | 28.7 | 7.4 |
| Mean | 19.1 | 19.7 | 0.6 | 19.7 | .6 | 18.6 | −0.4 |
| Median | 17.6 | 17.6 | −0.3 | 18.8 | −0.1 | 17.6 | 0.1 |
| Standard | 4.5 | 7.1 | 6.3 | 5.9 | 3.9 | 4.5 | 3.9 |
| Deviation | | | | | | | |
| Maximum | 28.9 | 38.4 | 17.1 | 31.0 | 9.7 | 28.7 | 7.4 |
| Minimum | 14.5 | 14.0 | −7.6 | 12.9 | −3.8 | 13.2 | −6.1 |
| Total # of | 10 | | | | | | |
| Subjects | | | | | | | |
| # of Increased | — | 3 | | 4 | | 6 | |
| Values | | | | | | | |
| # of Decreased | — | 6 | | 5 | | 4 | |
| Values | | | | | | | |
| # of Unchanged | — | 1 | | 1 | | 0 | |
| Values | | | | | | | |

Wrinkle depth volume (mm3) was measured for the heat-shocked ASC-derived exosome group and showed a mean value of 2.55 mm³ at baseline, 2.46 mm³ at week 1, 2.60 mm³ at week 2, and 2.55 mm³ at week 4. The mean percent change from baseline was −3.5% at week 1, 1.9% at week 2, and 0.0% at week 4.

At week 1, 6000 of subjects showed decreased wrinkle depth volume values (see, Table 25). Wrinkle depth volume represents the total amount of space of the wrinkle fold across a certain length, factoring in both its depth and its width.

TABLE 25
Wrinkle Depth Volume - ASC-Derived Exosomes: Difference from

| | | | Difference | | Difference | | Difference |
|---|---|---|---|---|---|---|---|
| Subject # | Baseline | Week 1 | Week 1 | Week 2 | Week 2 | Week 4 | Week 4 |
| 2 | 0.70 | 0.93 | 0.23 | 0.86 | 0.16 | 0.71 | 0.01 |
| 5 | 4.55 | 4.33 | −0.22 | 5.12 | 0.57 | 4.67 | 0.12 |
| 6 | 1.65 | 1.86 | 0.21 | 1.88 | 0.23 | 2.13 | 0.48 |
| 9 | 2.71 | 2.49 | −0.22 | 2.37 | −0.34 | 2.37 | −0.34 |
| 11 | 2.06 | 1.78 | −0.29 | 2.25 | 0.19 | 1.89 | −0.17 |
| 14 | 4.87 | 4.20 | −0.68 | 4.37 | −0.50 | 4.42 | −0.46 |
| 16 | 5.18 | 4.88 | −0.30 | 4.49 | −0.69 | 4.68 | −0.49 |
| 18 | 2.19 | 2.71 | 0.52 | 3.07 | 0.89 | 2.97 | 0.78 |
| 21 | 0.20 | 0.40 | 0.19 | 0.55 | 0.35 | 0.34 | 0.13 |
| 22 | 1.42 | 1.08 | −0.34 | 1.04 | −0.37 | 1.36 | −0.06 |
| Mean | 2.55 | 2.46 | −0.09 | 2.60 | 0.05 | 2.55 | 0.00 |
| Median | 2.13 | 2.17 | −0.22 | 2.31 | 0.17 | 2.25 | −0.03 |
| Standard | 1.76 | 1.56 | 0.36 | 1.62 | 0.51 | 1.60 | 0.41 |
| Deviation | | | | | | | |
| Maximum | 5.18 | 4.88 | 0.52 | 5.12 | 0.89 | 4.68 | 0.78 |
| Minimum | 0.20 | 0.40 | −0.68 | 0.55 | −0.69 | 0.34 | −0.49 |
| Total # of | 10 | | | | | | |
| Subjects | | | | | | | |
| # of Increased | — | 4 | | 6 | | 5 | |
| Values | | | | | | | |
| # of Decreased | — | 6 | | 4 | | 5 | |
| Values | | | | | | | |
| # of Unchanged | — | 0 | | 0 | | 0 | |
| Values | | | | | | | |

Wrinkle depth volume (mm³) was measured for the heat-shocked Aloe-derived exosomes group and showed a mean value of 2.77 mm³ at baseline, 2.97 mm³ at week 1, 2.90 mm³ at week 2, and 2.83 mm³ at week 4. The mean percentage change from baseline was 7.300 at week 1, 4.700 at week 2, and 2.00% at week 4. At week 1, 600% of subjects showed decreased wrinkle depth volume values (see, Table 26).

TABLE 26
Wrinkle Depth Volume - Aloe-Derived Exosomes: Difference from

| | | | Difference | | Difference | | Difference |
|---|---|---|---|---|---|---|---|
| Subject # | Baseline | Week 1 | Week 1 | Week 2 | Week 2 | Week 4 | Week 4 |
| 1 | 3.30 | 3.60 | 0.30 | 3.41 | 0.11 | 3.60 | 0.30 |
| 3 | 3.26 | 3.30 | 0.04 | 3.57 | 0.31 | 3.54 | 0.27 |
| 7 | 1.12 | 1.07 | −0.05 | 1.08 | −0.04 | 1.31 | 0.18 |
| 8 | 2.92 | 2.62 | −0.30 | 2.65 | −0.27 | 2.23 | −0.69 |
| 10 | 1.83 | 1.78 | −0.06 | 2.19 | 0.36 | 1.70 | −0.13 |
| 12 | 3.26 | 3.28 | 0.02 | 3.79 | 0.52 | 3.36 | 0.09 |
| 13 | 3.97 | 3.48 | −0.49 | 3.78 | −0.20 | 3.92 | −0.05 |
| 15 | 2.04 | 1.94 | 0.10 | 2.03 | −0.01 | 1.90 | −0.14 |
| 17 | 0.10 | 0.05 | −0.04 | 0.07 | −0.03 | 0.03 | −0.06 |
| 20 | 5.89 | 8.60 | 2.71 | 6.44 | 0.55 | 6.67 | 0.78 |
| Mean | 2.77 | 2.97 | 0.20 | 2.90 | 0.13 | 2.83 | 0.06 |
| Median | 3.09 | 2.95 | −0.05 | 3.03 | 0.05 | 2.79 | 0.02 |
| Standard | 1.61 | 2.29 | 0.91 | 1.75 | 0.29 | 1.82 | 0.38 |
| Deviation | | | | | | | |
| Maximum | 5.89 | 8.60 | 2.71 | 6.44 | 0.55 | 6.67 | 0.78 |
| Minimum | 0.10 | 0.05 | −0.49 | 0.07 | −0.27 | 0.03 | −0.69 |
| Total # of | 10 | | | | | | |
| Subjects | | | | | | | |
| # of Increased | — | 4 | | 5 | | 5 | |
| Values | | | | | | | |
| # of Decreased | — | 6 | | 5 | | 5 | |
| Values | | | | | | | |
| # of Unchanged | — | 0 | | 0 | | 0 | |
| Values | | | | | | | |

Wrinkle depth values (mm) were measured for the heat-shocked ASC-derived exosome group and showed a mean value of 0.066 mm at baseline, 0.065 mm at week 1, 0.065 mm at week 2, and 0.065 mm at week 4. The mean percent change from baseline was −0.5% across all weeks. At week 1, 700% of subjects showed decreased wrinkle depth values (see, Table 27). Wrinkle depth values (mm) represent the depth of a skin crease perpendicularly from the surface.

TABLE 27
Wrinkle Depth Value- ASC-Derived Exosomes: Difference from

| | | | Difference | | Difference | | Difference |
|---|---|---|---|---|---|---|---|
| Subject # | Baseline | Week 1 | Week 1 | Week 2 | Week 2 | Week 4 | Week 4 |
| 2 | 0.053 | 0.056 | 0.002 | 0.055 | 0.002 | 0.054 | 0.001 |
| 5 | 0.079 | 0.077 | −0.002 | 0.087 | 0.009 | 0.075 | −0.004 |
| 6 | 0.062 | 0.059 | −0.002 | 0.061 | −0.001 | 0.065 | 0.003 |
| 9 | 0.069 | 0.066 | −0.003 | 0.064 | −0.005 | 0.067 | −0.002 |
| 11 | 0.061 | 0.065 | 0.004 | 0.065 | 0.003 | 0.065 | 0.003 |
| 14 | 0.071 | 0.061 | −0.010 | 0.066 | −0.005 | 0.064 | −0.006 |
| 16 | 0.066 | 0.067 | 0.002 | 0.066 | 0.000 | 0.064 | −0.001 |
| 18 | 0.089 | 0.082 | −0.006 | 0.080 | −0.009 | 0.078 | −0.011 |
| 21 | 0.051 | 0.051 | 0.000 | 0.053 | 0.002 | 0.052 | 0.001 |
| 22 | 0.064 | 0.063 | −0.001 | 0.057 | −0.007 | 0.062 | −0.002 |
| Mean | 0.066 | 0.065 | −0.002 | 0.065 | −0.001 | 0.065 | −0.002 |
| Median | 0.065 | 0.064 | −0.002 | 0.064 | 0.000 | 0.064 | −0.001 |
| Standard | 0.011 | 0.009 | 0.004 | 0.011 | 0.005 | 0.008 | 0.004 |
| Deviation | | | | | | | |
| Maximum | 0.089 | 0.082 | −0.004 | 0.087 | 0.009 | 0.078 | 0.003 |
| Minimum | 0.051 | 0.051 | −0.010 | 0.053 | −0.009 | 0.052 | −0.011 |
| Total # of | 10 | | | | | | |
| Subjects | | | | | | | |
| # of Increased | — | 3 | | 5 | | 4 | |
| Values | | | | | | | |
| # of Decreased | — | 7 | | 5 | | 6 | |
| Values | | | | | | | |
| # of Unchanged | — | 0 | | 0 | | 0 | |
| Values | | | | | | | |

Wrinkle depth values (mm) were measured for the heat-shocked Aloe-derived exosome group and showed a mean value of 0.064 mm at baseline, 0.064 mm at week 1, 0.064 mm at week 2, and 0.064 mm at week 4.

The mean percent change from baseline was 0% across all weeks. At week 1, 70% of subjects showed decreased wrinkle depth values (see, Table 28). Wrinkle depth values (mm) represent the depth of a skin crease perpendicularly from the surface.

TABLE 28
Wrinkle Depth Values- Aloe-Derived Exosomes: Difference from

| | | | Difference | | Difference | | Difference |
|---|---|---|---|---|---|---|---|
| Subject # | Baseline | Week 1 | Week 1 | Week 2 | Week 2 | Week 4 | Week 4 |
| 1 | 0.071 | 0.071 | 0.000 | 0.076 | 0.005 | 0.074 | 0.003 |
| 3 | 0.063 | 0.061 | −0.002 | 0.063 | 0.001 | 0.064 | 0.001 |
| 7 | 0.055 | 0.060 | 0.005 | 0.056 | 0.001 | 0.055 | −0.001 |
| 8 | 0.058 | 0.058 | −0.001 | 0.060 | 0.001 | 0.058 | 0.000 |
| 10 | 0.056 | 0.057 | 0.001 | 0.057 | 0.001 | 0.060 | 0.004 |
| 12 | 0.065 | 0.067 | 0.002 | 0.071 | 0.006 | 0.068 | 0.003 |
| 13 | 0.072 | 0.068 | −0.004 | 0.073 | 0.002 | 0.072 | 0.001 |
| 15 | 0.060 | 0.058 | −0.002 | 0.058 | −0.002 | 0.059 | −0.002 |
| 17 | 0.052 | 0.051 | −0.001 | 0.047 | −0.005 | 0.049 | −0.003 |
| 20 | 0.090 | 0.085 | −0.004 | 0.081 | −0.009 | 0.082 | −0.008 |
| Mean | 0.064 | 0.064 | −0.001 | 0.064 | 0.000 | 0.064 | 0.000 |
| Median | 0.062 | 0.060 | −0.001 | 0.061 | 0.001 | 0.062 | 0.000 |
| Standard | 0.011 | 0.010 | 0.003 | 0.011 | 0.004 | 0.010 | 0.003 |
| Deviation | | | | | | | |
| Maximum | 0.090 | 0.085 | 0.005 | 0.081 | 0.006 | 0.082 | 0.004 |
| Minimum | 0.052 | 0.051 | −0.004 | 0.047 | −0.009 | 0.049 | −0.008 |
| Total # of | 10 | | | | | | |
| Subjects | | | | | | | |
| # of Increased | — | 3 | | 7 | | 6 | |
| Values | | | | | | | |
| # of Decreased | — | 7 | | 3 | | 4 | |
| Values | | | | | | | |
| # of Unchanged | — | 0 | | 0 | | 0 | |
| Values | | | | | | | |

### Skin Firmness and Elasticity

Skin firmness and elasticity was tested using a Cutometer®, which is a specialized, noninvasive device used to measure skin's elasticity and firmness by applying negative pressure (e.g., suction) to deform the skin. It calculates the skin's ability to return to its original position through an optical system.

A vacuum draws the skin into a 2 mm-8 mm probe aperture for a set time, after which the suction releases and the skin recovers. It measures maximum deformation (firmness) and the ability to return to its original shape (elasticity), displaying the results as graphs and numerical parameters (R parameters). R0 refers to final distention/firmness (i.e., lower values indicate higher firmness (less final distention)), wherein R2 refers to gross elasticity (i.e., higher values signify better overall elasticity).

Cutometer® measurements demonstrated that 100% of subjects in the heat-shocked ASC-derived exosome group had a statistically significant increase in skin firmness (see, Table 29).

TABLE 29
Cutometer ® Skin Firmness (R0) and
Elasticity (R2) - ASC-Derived Exosomes

| | Best | Response | Best | Response |
|---|---|---|---|---|
| | Cutometer | Week | Cutometer | Week |
| Subject # | R0 | R0 | R2 | R2 |
| 2 | −0.151 | 1*, 2, 4 | 0.029 | 4* |
| 5 | −0.113 | 1, 2, 4* | NO | — |
| 6 | −0.127 | 1, 2*, 4 | 0.181 | 1*, 2, 4 |
| 9 | −0.05 | 1, 2*, 4 | 0.107 | 1*, 4 |
| 11 | −0.072 | 1, 2*, 4 | NO | — |
| 14 | −0.123 | 1*, 2, 4 | NO | — |
| 16 | −0.04 | 1*, 2, 4 | 0.177 | 1, 2, 4* |
| 18 | −0.093 | 1, 2*, 4 | 0.149 | 1*, 4 |
| 21 | −0.131 | 1, 2, 4* | 0.155 | 1, 2*, 4 |
| 22 | −0.128 | 1, 2, 4* | NO | — |

*Asterisk indicates the week at which the best (statistically significant) response was observed.
“NO” indicates no statistically significant change was observed for that parameter.

For the heat-shocked Aloe-derived exosome group, a statistically significant increase in skin firmness was also observed (see, Table 30).

TABLE 30
Cutometer ® Skin Firmness (R0) and
Elasticity (R2) - Aloe-Derived Exosomes

| | Best | Response | Best | Response |
|---|---|---|---|---|
| | Cutometer | Week | Cutometer | Week |
| Subject # | R0 | R0 | R2 | R2 |
| 1 | 0.31 | 2* | 0.127 | 1*, 4 |
| 3 | — | — | — | — |
| 7 | 0.066 | 1*, 2, 4 | 0.048 | 2*, 4 |
| 8 | 0.01 | 4* | 0.112 | 2* |
| 10 | 0.039 | 1*, 2, 4 | 0.103 | 1, 2* |
| 12 | 0.033 | 1*, 2, 4 | — | — |
| 13 | 0.002 | 2, 4* | 0.156 | 2, 4* |
| 15 | 0.029 | 2, 4* | 0.34 | 1* |
| 17 | 0.068 | 1, 2* | — | — |
| 20 | 0.03 | 1, 2, 4* | 0.053 | 1*, 2 |

*Asterisk indicates the week at which the best (statistically significant) response was observed.
“—” indicates data not available for that parameter.

### Skin Luminosity

Skin luminosity was assessed by VISIA CR® imaging analysis. VISIA CR® is a skin imaging system that uses multispectral imaging to capture, analyze, and quantify skin surface and subsurface conditions under different lighting modes. VISIA CR® measures the overall brightness and radiance of the skin surface. An increase in luminosity value indicates improved skin radiance. The luminosity increase value represents the magnitude of improvement observed, and the response weeks indicate the treatment weeks at which the luminosity increase was detected.

In the heat-shocked ASC-derived exosome group, 4 of 10 subjects (400%) demonstrated a measurable increase in skin luminosity, with responses observed at weeks 2 and 4 of treatment (see, Table 31).

TABLE 31
Skin Luminosity - ASC-Derived Exosomes

| Subject # | Luminosity Increase | Response Weeks |
|---|---|---|
| 2 | 2 | 2, 4 |
| 5 | — | — |
| 6 | — | — |
| 9 | 2 | 2, 4 |
| 11 | — | — |
| 14 | — | — |
| 16 | 1 | 2, 4 |
| 18 | 2 | 2, 4* |
| 21 | — | — |
| 22 | — | — |

*Asterisk indicates the week at which the best (statistically significant) response was observed.
“—” indicates no available data.

In the heat-shocked Aloe-derived exosome group, 6 subjects (60%) demonstrated a measurable increase in skin luminosity and responses observed at weeks 2 and 4 of treatment (see, Table 32).

TABLE 32
Skin Luminosity - Aloe-Derived Exosomes

| Subject # | Luminosity Increase | Response Weeks |
|---|---|---|
| 1 | — | — |
| 3 | 1 | 4* |
| 7 | — | — |
| 8 | 2 | 2*, 4 |
| 10 | — | — |
| 12 | — | — |
| 13 | 1 | 2, 4 |
| 15 | 1 | 2, 4 |
| 17 | 1 | 2, 4 |
| 20 | 2 | 4* |

*Asterisk indicates the week at which the best (statistically significant) response was observed.
“—” indicates no available data.

### Subject Questionnaire Results

Subject questionnaires were administered for subjects to self-evaluate their subjective perceptions of product efficacy. The questionnaire included the following options: (1) skin appears healthier and younger looking; (2) skin feels more hydrated; (3) test product improves skin's softness and smoothness; (4) test product reduces roughness and dryness; (5) test product improves skin's radiance; (6) test product improves skin's overall appearance; (7) test product improves skin's brightness; (8) test product rejuvenates skin; (9) test product nourishes skin; (10) skin texture is improved; (11) face appears more luminous; (12) skin feels firmer; (13) skin looks tighter; (14) test product is gentle enough for everyday use; (15) test product absorbs easily into skin; (16) subject likes the clean fragrance free scent; and (17) subject would recommend the product to a friend.

At week 4 after using the test products, 100% of subjects agreed or strongly agreed for all 17 questions.

### Clinical Case Reports

In addition to the quantitative measurements described above, clinical case reports were documented through photographic evidence to visually assess the effects of topical application of the exosome-containing formulations on various skin conditions.

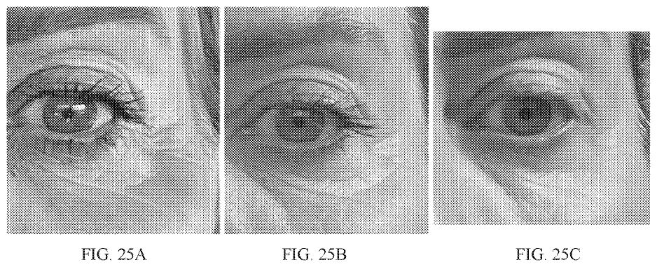

In a first case report, a subject with visible crow's feet wrinkles in the periorbital region was treated with the exosome-containing moisturizer. Before treatment, the periorbital skin exhibited visible fine lines and wrinkles radiating outward from the outer corner of the eye, along with surface roughness and textural irregularities (see, FIG. 25A). Following treatment, the periorbital skin demonstrated a reduction in the appearance of fine lines and wrinkles in the crow's feet region, with improved skin smoothness and texture (see, FIGS. 25A-25C).

FIG. 25A, FIG. 25B, and FIG. 25C show photographs of a subject with visible crow's feet wrinkles in the periorbital region before and after treatment with a moisturizer comprising tuned ASC-derived exosomes. FIG. 25A shows the periorbital skin before treatment. FIG. 25B shows the periorbital skin during the course of treatment. FIG. 25C shows periorbital skin after treatment, indicating improvement over the course of the treatment.

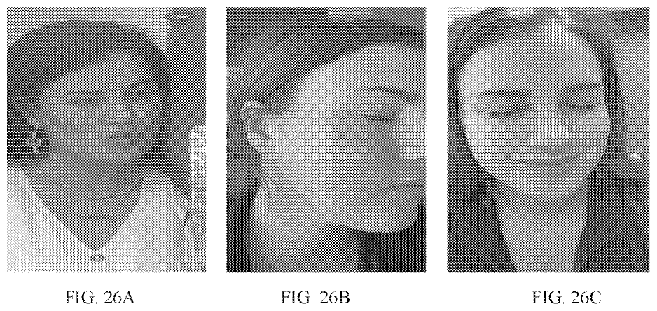

In a second case report, a subject with visible acne on the face characterized by blemishes and uneven skin texture across the cheek and jawline was treated with the tuned ASC-derived exosome-containing moisturizer (see, FIG. 26A). Following treatment, the subject's skin demonstrated a significant reduction in redness and inflammation, with improved skin clarity and a more uniform skin tone (see, FIGS. 26B-26C).

FIG. 26A, FIG. 26B, and FIG. 26C show photographs of a subject with visible acne on the face before and after treatment with a moisturizer comprising tuned ASC-derived exosomes. FIG. 26A shows the face before treatment. FIG. 26B shows the face during the course of treatment. FIG. 26C shows face after treatment, indicating improvement over the course of the treatment.

In a third case report, a before-and-after comparison of the face of a subject demonstrated a significant reduction in acne following treatment with the tuned ASC-derived and Aloe-derived exosome-containing formulation. The post-treatment photographs showed marked improvement in acne following 7 days of treatment (see, FIGS. 27A-27B).

FIG. 27A and FIG. 27B show photographs of a subject with visible acne on the face before and after treatment with a moisturizer comprising ASC-derived and Aloe-derived exosomes. FIG. 27A shows the face before treatment. FIG. 27B shows the face following 7 days of treatment, indicating improvement over the course of the treatment.

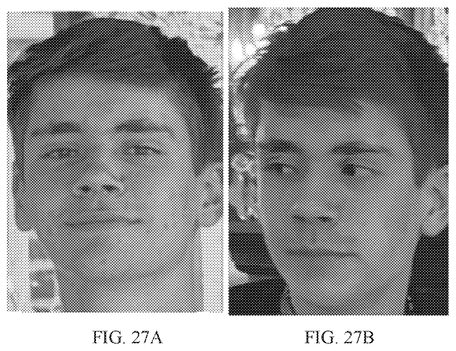

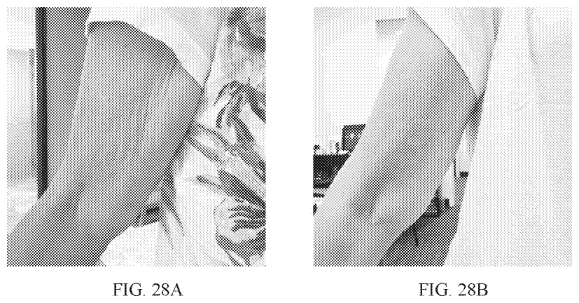

In a fourth case report, a subject demonstrated improved skin hydration and overall skin firmness on the upper arm following treatment with the tuned ASC-derived exosome-containing topical formulation. The skin surface showed visible improvements in hydration and firmness following a 2 week treatment course (see, FIGS. 28A-28B).

FIG. 28A and FIG. 28B show photographs of a subject with improved skin hydration and overall skin firmness on the upper arm following treatment with the tuned ASC-derived exosome-containing topical formulation. FIG. 28A shows the upper arm before treatment. FIG. 28B shows the upper arm following 2 weeks of treatment, indicating improvement over the course of the treatment.

These clinical case reports provide visual evidence supporting the quantitative clinical study findings that topical compositions comprising tuned exosomes can improve skin appearance across multiple parameters including wrinkle reduction, reduction of redness and inflammation, improved skin texture and clarity, enhanced hydration, and overall skin rejuvenation.

### Example 5: Clinical Evaluation of Topical Exosome Compositions for Eczema Treatment

### Rationale

Bioactive exosomes from ASCs can be tuned, purified, and delivered to the skin via topical emulsions to reprogram skin cells and reduce the symptoms associated with eczema. A scalable bioprocess was developed to engineer pro-healing exosomes specifically tuned to target inflammatory skin diseases and enhance healing processes. By controlling the cellular environment in a closed-system bioreactor, production and release of exosomes that are biologically active and configured with a distinct miRNA cargo may be stimulated.

Tuned ASC-derived exosomes contain an activity profile that reduces inflammatory cytokines, stimulates collagen production, and activates keratinocyte and fibroblast proliferation. In the context of eczema, decreased levels of specific miRNAs, such as miR-29b and miR-143, are often observed in lesional skin, which contributes to the disrupted skin barrier function and increased inflammation characteristics of the disease. Low expression of miR-29b and miR-143 may exacerbate symptoms. An ASC-derived miRNA cargo signature was identified which includes miR-29b and miR-143. Replenishing decreased levels of protective miRNAs via exosomal cargo may offer a treatment strategy for eczema.

### ASC-Derived Exosome Characterization

Manufactured extracellular vesicles isolated from cultured ASCs were characterized using the minimal information for studies of extracellular vesicles (MISEV) guidelines for extracellular vesicle classification. The MISEV guidelines are a set of international consensus standards published by the International Society of Extracellular Vesicles (ISEV) (see, Welsh, J. A., et al. Minimal information for studies of extracellular vesicles (MISEV2023): from basic to advanced approaches. Journal of Extracellular Vesicles, 13, e12404).

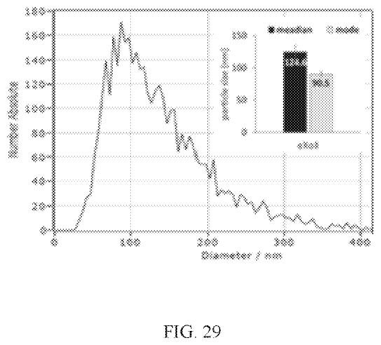

Nanoparticle Tracking Analysis (NTA) was used to determine particle size distribution. It showed that the diameter of ASC-derived exosomes is less than about 100 nanometers (see, FIG. 29).

The insert to FIG. 29 is a graph depicting the size of tuned ASC-derived exosomes (eXo3), as measured by nanoparticle tracking analysis (NTA), indicating a median diameter of about 124.6 nm and a mode diameter of 90.5 nm.

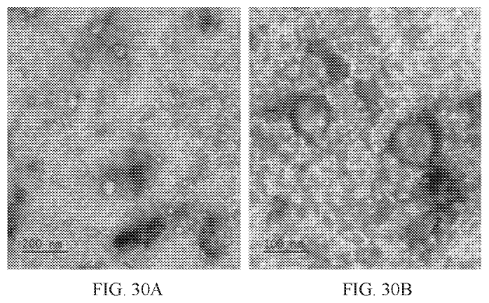

Transmission Electron Microscopy (TEM) provided direct visual confirmation of exosome morphology and size, confirming that ASC-derived exosomes are less than 100 nanometers in diameter (see, FIGS. 30A-30B).

FIG. 30A and FIG. 30B are transmission electron microscopy (TEM) images depicting tuned ASC-derived (eXo3) exosome morphology. FIG. 30A shows a zoomed-out TEM image, scale bar indicating 200 nm. FIG. 30B shows a magnified TEM image, indicating that the tuned ASC-derived exosomes are about 100 nm in diameter, scale bar indicating 100 nm.

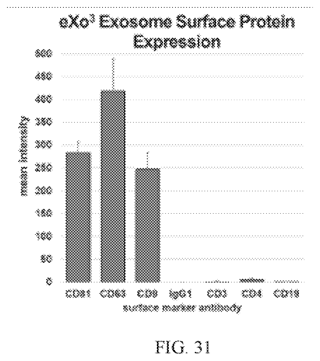

Immunofluorescence demonstrated expression of canonical exosome surface protein markers CD9, CD63, and CD81 on the ASC-derived exosomes (see, FIG. 31).

FIG. 31 shows a bar graph demonstrating an immunofluorescence quantification showing expression of exosome surface protein markers detected by antibodies on the tuned ASC-derived exosomes (eXo3), including CD81, CD63, and CD9.

miRNA Cargo Characterization

Full protein characterization and miRNA sequencing of tuned ASC-derived exosomes was conducted via RNA-Sequencing (RNA-Seq), revealing the full transcriptome of ASC-derived exosomes (as shown in Example 3). The miRNA cargo of tuned ASC-derived exosomes was characterized and categorized according to functional relevance in eczema treatment applications.

Table 33 below presents miRNAs identified in tuned ASC-derived exosomes with significance to eczema treatment.

TABLE 33
miRNAs with Significance to Eczema Treatment

| miRNA | Significance |
|---|---|
| miR-143 | Reduces bacterial induced inflammation; reduces TLR2 protein |
| miR-146a-5p | Decreases IL-8 in skin lesions; suppresses Th1 type responses in |
| | skin |
| miR-21-5p | Regulates healthy aging phenotype in skin |

### Functional Analysis of ASC-Derived Exosomes

### Anti-Inflammatory Activity

To measure EV effects on inflammation, in vitro gene expression for the inflammatory cytokines IL-6, IL-8 and MCP-1 was performed via RT-qPCR. Primary human fibroblasts (PHFs) were seeded at a density of 390,000 cells per well in growth medium (DMEM-LG, 10% FBS and antibiotics) using 6-well plates. The cells were incubated overnight to allow the PHFs to attach.

To induce inflammatory cytokine production, growth medium was removed and replaced with Test Medium (DMEM-HG, ascorbic acid and antibiotics) containing 1×10E7/mL heat killed P. gingivalis (HKPG, InvivoGen®) with and without 6.7 μg/mL ASC-derived EVs. After overnight incubation, the cells were washed and RNA extracted using RNeasy® Mini Kit (Qiagen®). cDNA was prepared using 1 μg of purified RNA in a 20 μL reaction and High-Capacity cDNA Reverse Transcription Kit (Life Technologies®). qPCR was performed with 10 ng cDNA per reaction using iTaq™ Universal Probes Master Mix (BioRad®) and a QuantStudio® 12K Flex machine (Life Technologies®).

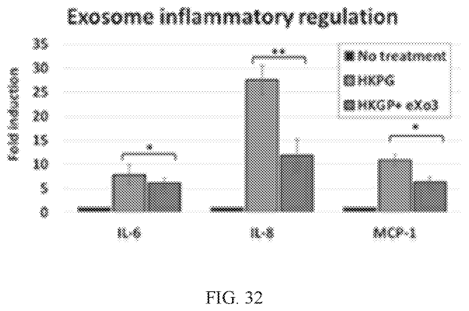

FIG. 32 shows bar graphs of fold induction (y-axis) vs treatment (x-axis) depicting levels of the inflammatory cytokines IL-6, IL-8, and monocyte chemoattractant protein-1 (MCP-1) following treatment of human fibroblasts with tuned ASC-derived exosomes (eXo3), P. gingivalis (HKPG), and a no treatment control for 12 hours.

Human fibroblasts treated with P. gingivalis and tuned ASC-derived exosomes for 12 hours showed a reduction of inflammatory cytokines compared to P. gingivalis alone, as measured by RT-qPCR. The data indicated that tuned ASC-derived exosome treatment reduces the secretion of inflammatory cytokines and chemokines MCP-1, IL-6, and IL-8 compared to P. gingivalis treatment alone (see, FIG. 32). P. gingivalis induces inflammatory response by upregulating chemokine and cytokine production.

### Clinical Case Studies

Under informed consent, tuned ASC-derived exosomes formulated in emulsion compositions were evaluated when topically applied to afflicted skin. The tuned ASC-derived exosomes showed a dramatic reduction of eczema symptoms including discomfort, redness, scaling, and itchiness.

### Clinical Case Study 1

A subject with a 15-year history of intractable eczema lesion on the leg was evaluated. The subject had been resistant to all treatments including both topical and oral steroids as well as immunomodulators and many over-the-counter therapies. The subject applied tuned ASC-derived exosome body lotion topically twice daily to the left leg.

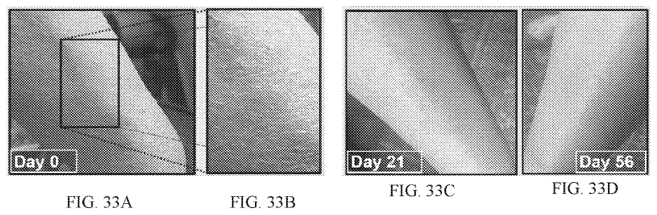

Within the first week of treatment, the subject reported a dramatic reduction in itchiness and pain associated with the eczema rash. The raised plaque was diminished by the third week and completely resolved after fifty-six days without further redness or discoloration (see, FIGS. 33A-33D). At the fifty-two week follow-up, the subject reported no recurrences, discomfort, itchiness, or pain.

FIG. 33A, FIG. 33B, FIG. 33C, and FIG. 33D show photographs of a left leg of a subject with visible eczema before and after twice-daily treatment with a topical lotion emulsion formulation comprising tuned ASC-derived exosomes indicating improvement over the course of the treatment. FIG. 33A shows the leg before treatment. FIG. 33B shows a magnified image of the leg before treatment indicating visible eczema. FIG. 33C shows the leg 21 days after treatment. FIG. 33D shows leg 56 days after treatment.

### Clinical Case Study 2

A subject with eczema lesions located behind the knee was evaluated. The subject applied tuned ASC-derived exosome face moisturizer topically behind the knee twice a day.

In the first week after applying tuned ASC-derived exosome face moisturizer, a dramatic improvement in discomfort was reported by the subject. During the third week, there was a noticeable decrease in lesion size, and the subject continued to report a reduction in pain and discomfort (see, FIGS. 34A-34B). After 6 weeks, the scale and raised nature of the lesions completely resolved with only remaining surface redness noted at the margins.

FIG. 34A and FIG. 34B show photographs of a subject with visible eczema on the back of the knee before and after twice-daily treatment with a topical lotion emulsion formulation comprising tuned ASC-derived exosomes. FIG. 34A shows the back of the knee before treatment. FIG. 34B shows the back of the knee 18 days after treatment, indicating improvement over the course of the treatment.

### SUMMARY

Topically applied tuned ASC-derived exosomes decreased inflammation and stimulated tissue repair mechanisms that provided treatment for intractable eczema. The clinical case studies demonstrated that tuned ASC-derived emulsion compositions, when applied topically to eczema-afflicted skin, resulted in reduction of eczema symptoms including discomfort, redness, scaling, and itchiness. The findings support tuned exosomes having targeted activity and usefulness as miRNA replacement therapies that alleviate symptoms and provide overall skin health improvement.

### Example 6: Modulation of ASC-Derived Extracellular Vesicles Containing Cargo That Enhances Wound Healing

### Rationale

A bioreactor-based production system was developed for manufacturing ASC-derived exosomes using a highly controlled and tunable environment that can modify the cargo of these nanovesicles. The innovation focuses on tuning pro-healing EVs with therapeutic activity and using a topical formulation to treat diabetic ulcers.

To evaluate biological activity of tuned ASC-derived exosomes, functional activity assays were performed using human primary dermal fibroblast and keratinocyte culture models. Molecular and biochemical assays were used to assess cytokine regulation, collagen production and cell migration. (see above) Rodent wound healing models were used to assess therapeutic potential of tuned ASC-derived exosomes. (see below) A human volunteer case study was carried out with a consenting individual suffering from chronic diabetic ulcers.

The data presented herein demonstrates that the tuned ASC-derived exosomes, contain an activity profile that reduces inflammatory cytokines, stimulates collagen production, and activates keratinocyte and fibroblast proliferation and migration. When formulated with an emollient and topically applied to an in vivo excisional wound model, tuned ASC-derived exosomes demonstrated enhanced wound closure, increased keratinization, collagen deposition, and overall improved recovery rate. In a clinical case study addressing non-healing diabetic foot ulcers, conducted under informed consent, topical treatment with tuned ASC-derived exosomes formulated in a proprietary gel serum showed complete wound closure and dermal regeneration.

The research efforts described herein resulted in an EV manufacturing system that can be directed to improve the healing capacity of ASC-derived exosomes. Enhanced wound healing and repair activity was demonstrated in vitro and in vivo. The data supports the regenerative properties of exosomes and reinforces their strong therapeutic potential.

### Introduction

Intercellular and extracellular communication is essential for maintaining homeostasis in multicellular organisms, especially in response to tissue injury. One key mode of communication involves extracellular vesicles (EVs) that serve as vehicles to facilitate the exchange of bioactive cargo, including proteins, RNAs, and lipids, that influence target cell behavior (Zhang et al., 2019). Current research has shown that EVs are secreted by all cells and provide paracrine communication by employing different targeting mechanisms as well as by carrying different cargos to effector cells (Gurunathan et al., 2021; Zou et al., 2023). The underlying processes involved in regulating secretion and packaging of vesicular content are not well understood, but harnessing these bioactive systems provides an attractive modality for enhancing the regenerative capacity of innate biological material. Many studies have demonstrated that EVs are differentially produced and secreted according to the cellular environment (Kowal et al., 2014; Pote and Gacche, 2025; Skotland et al., 2019).

The present disclosure builds upon this premise by developing a bioreactor-based production system for manufacturing ASC-derived exosomes using a highly controlled and tunable environment that can modify EV production and cargo that reduces inflammatory cytokines, stimulates collagen production, as well as activates keratinocyte and fibroblast proliferation and migration (Zhang et al., 2015; Zhang et al., 2020). When formulated with an emollient and topically applied to an in vivo excisional wound model, tuned ASC-derived exosomes demonstrated enhanced wound closure, increased keratinization, collagen deposition, and overall improved recovery rate. In a clinical case study addressing non-healing diabetic foot ulcers, conducted under informed consent, topical treatment with tuned ASC-derived exosomes formulated in a proprietary gel serum showed complete wound closure and dermal regeneration.

### Materials and Methods

### ASC Culturing and Exosome Purification

Research has shown that both exosome cargos and stem cell paracrine activity are dramatically altered by the environment, particularly with regard to the inflammatory and healing state of tissue (Shabbir et al., 2015; Shi et al., 2021; Rani and Ritter, 2016). Critical ex vivo environmental cues can tune ASC-derived exosomes to be pro-healing. A bioreactor-based production system was developed for manufacturing “tuned” EVs from ASCs. By manipulating the cellular bioreactor environment, it was found that ASC EV production and secretion as well as exosomal cargo packaging can be altered. Additionally, this manipulation of the bioreactor environment was shown to improve the pro-healing capacity of the ASC-derived exosomes. Isolation and culturing of the ASCs, and exosome isolation and physical characterization, have been previously reported (Ludlow and Buehrer, 2024). The results depicted and described herein characterize enhanced features of ASC-derived exosomes by comparing to “native” exosomes which were prepared in tandem from the same lot of ASCs cultured in an identical bioreactor system under traditional culturing conditions with no modification steps.

It is important to maintain a controlled environment while manufacturing EVs that retain wound healing properties. An aging microenvironment will promote cellular senescence that can shift EV production to pro-inflammatory characteristics that negatively affect tissue repair and exacerbate cellular pathology (Pirmoradi et al., 2018; Smirnova et al., 2023; Liu et al., 2017). The secretome of ASCs with senescence-associated secretory phenotype (SASP) generates a proinflammatory microenvironment affecting surrounding cells (Siraj et al., 2023). ASCs are more resistant to senescence shifts and comparative studies show long-term cultures have higher proliferation rates and decreased oxidative stress (Wagner et al., 2005; Wagner et al., 2008; Gruber et al., 2012). Additionally, hollow-fiber bioreactor systems retain differentiation capacity, surface phenotype, genetic stability, and show no evidence of increased aging after large-scale culture (Vymetalova et al., 2020). The commercial-scale ASC bioreactor process monitors glucose consumption, cell proliferation, and cell cycle to ensure that the cells are not senescent, thus safeguarding against SASP vesicle formation which may compromise the efficacy of the product.

Briefly, exosomes were isolated from ASCs using a hollow-fiber cartridge containing bioreactor (Ludlow and Buehrer, 2022). The ASCs used were isolated from human adipose tissue and expanded in culture (Buehrer and Cheatham, 2013). To prepare the bioreactor, ASCs (750 million-1 billion ASCs) from multiple donors (1-4 donors) with similar demographics were seeded onto the cartridge. After seeding, a separate cell cartridge was filled with serum-free medium for ASC maintenance and EV production, and growth medium was continually circulated through the bioreactor with medium exchanges every 3-5 days. Bioreactor cultures can be maintained in this manner for up to 3 months while the ASCs retain their ISCT-mandated MSC characteristics.

EVs were collected every 3-5 days after the bioreactor culture was established. The conditioned serum-free medium was removed from the cell cartridge and subjected to two centrifugation steps as follows: 300×g for 5 minutes followed by 3000×g for 20 minutes. The supernatant was aspirated, and the pelleted EVs containing exosomes were suspended in DPBS and stored at −80 °C for further analysis.

### EV Characterization Methods

Particle size and concentration was determined using ZetaView® NTA (Particle Metrix) instrumentation. Concentrated EV preparations were diluted in particle free water to 5×10E7 particles per milliliter and analyzed using defined acquisition settings. Size distribution was determined and mean, median, and mode diameters were used to characterize EVs.

Transmission electron microscopy (TEM) was performed as follows: Size 300 mesh copper EM grids were glow-discharge formvar/carbon film coated for 45 seconds. Once completed, 5 μL of the EV suspension (previously fixed with 30 μL of 2% paraformaldehyde (PFA) was added to the grid and incubated for 1 minute. Following removal of the excess EV solution, one drop of filtered 1% uranyl acetate was added and the excess removed. The grid was rinsed on a drop of water 3 times. The grids were allowed to dry for 10 minutes at room temperature followed by imaging on Tecnai 12 electron microscope at 120 kV.

EV surface markers were analyzed using a Miltenyi MACSQuant® Analyzer 10 for sample acquisition and MACSQuantify® Software for data analysis. The MACSPlex® Exosome Kit (Miltenyi Biotec) was used to evaluate each sample for the expression of 37 surface markers; the MACSPlex® EV Kit IO comprises a cocktail of various fluorescently labeled bead populations, each coated with a specific antibody binding the respective surface epitopes. The 39 bead populations can be distinguished by different fluorescence intensities via flow cytometry. Extracellular vesicles were incubated with the antibody-coated MACSPlex®® EV Capture Beads. Subsequently or in parallel, EVs bound to the MACSPlex® EV Capture Beads were labeled with the MACSPlex® EV Detection Reagents. The MACSPlex® EV Detection Reagents can also be combined to create a cocktail comprising of MACSPlex® EV Detection Reagent for CD9, CD63, and CD81. Consequently, sandwich complexes were formed between the MACSPlex® EV Capture Bead, extracellular vesicle, and the detection reagent. These complexes can be analyzed based on the fluorescence characteristics of both the MACSPlex® EV Capture Bead and the detection reagent.

In brief, protein concentration was determined for each sample using a bicinchoninic acid (BCA) protein quantification kit (ThermoFisher®). 20 μg of prepared exosomes were gently rotated at 4 °C overnight with capture beads. The beads were washed and then incubated for 1 hour with a detection bead mixture consisting of CD9, CD63 and CD81 exosome markers. The flow cytometer was calibrated and background settings were adjusted to unlabeled beads; gating strategies were used to identify bead populations for each analyte. Batch analysis quantified median intensities for each bead population and analyte surface expression was calculated for each sample.

In vitro Migration Assay

Briefly, Radius™ Cell Migration Assay plates (i.e., biocompatible, hydrogel coated tissue culture plates) were prepared and human primary skin epithelial cells were seeded (100,000 cells/well) into a 24-well Radius™ plate in 10% serum containing media (1 mL). Following a 4 hour incubation at 37 °C in a 5% CO₂-containing atmosphere to allow cells to attach, wells were washed and the hydrogel dissolved from the wells. The wells were washed again, and either serum free, 1% serum containing media, 10% serum containing media, or 1% serum containing media supplemented with 50 μL of the indicated number of EVs was added. Photographs of each well were taken at T=48 following addition of EVs.

In vitro Cell Proliferation Assay

EVs were also added to low density in vitro primary human fibroblasts (PHF) and primary human keratinocytes (PHKs) cultures (3,000 cells/well) in 96-well culture plates using serum-free medium and incubated for 3 days. To compare the proliferative effects of EVs, cells were treated with other inducers, including 10% FBS, PDGF-BB, KGF, TGF-β 1, or IGF-1. After 3 days, the cells were treated with CellTiter-Blue® reagent (Promega Corporation) for 2 hours to assess proliferation by fluorescence signal (excitation: 560 nm, emission: 590 nm).

In vitro Collagen Production Assay

Dermal fibroblasts were seeded at 10,000 cells per well and allowed to attach overnight. The next day, cells were treated with EVs and controls in assay medium+/−ascorbic acid. Cells were treated for 48 hours in replicates of 3. After 48 hours, conditioned media was collected and frozen at −80 °C until ready to assay. Procollagen-1 levels were determined by ELISA (Takara Bio®, cat #MIK101).

### Excisional Wound Model

All animal husbandry was done in accordance with The Guide for the Care and Use of Laboratory Animals published by the Institute of Laboratory Animal Resources Commission on Life Science National Research Council, NATIONAL ACADEMY PRESS, Eighth Edition, and applicable USDA regulations without exception. Animals were housed on site (Synchrony Laboratories, Durham, NC, Study 169-03-17) for a minimum of 5 days prior to study assignment. Animals were handled by staff daily to acclimate to human contact and to reduce stress. Appropriate standard operating procedures (SOPs) and protocols were followed for housing conditions, health status review, diet, pre-op instructions, and post-operative care.

Marking and identification of animals was achieved by two non-invasive methods: (1) a black sharpie marker was used to inscribe a number on the animal's fur; and (2) each animal was housed individually with identification number affixed to the cage. Both circular and linear skin injury models were developed and investigated. For circular wounds, 4×2 cm diameter full-thickness wounds (extending through the dermis and into subcutaneous tissue) were cut on the back of each animal. Sterile saline (phosphate buffered, PBS) was added to each wound and the injury covered with a transparent sterile, gas-permeable bandage or surgical tape.

Each wound was measured and photographed daily for up to 30 days post-surgery. For linear wounds, 4×2 cm linear full-thickness cuts were made in the back skin. Sterile saline (phosphate buffered, PBS) was added to each wound and the injury covered with a transparent sterile, gas-permeable bandage or surgical tape. Each wound was measured and photographed daily for up to 30 days post-surgery.

Baseline wound healing rates were established using measurements derived from these preliminary studies. Circular or linear wound healing models were generated on back skin of rodents as described above. Of the 4 wounds per rat, one was treated with PBS as a vehicle control. The other 3 were treated with a heat shocked or non-heat shocked exosome preparation derived from ASC conditioned media.

The treatments were randomized across subjects to avoid positional bias and assessment was blinded to avoid observer bias. Matched conditions on the same animal improved statistical power by allowing paired analysis. Up to 4 experimental groups (with different exosome preparations) were tested, 6 rodents per group. All rodents were evaluated for wound healing response as described above. At the end of the study period, rodents were euthanized and the wound site dissected out for evaluation by molecular and histological techniques.

### Topical Formulation (Test Article)

ASC-derived exosomes are registered under International Nomenclature of Cosmetic Ingredients (INCI) name: Human Adipose Stem Cell Exosome. ASC-derived exosomes personal care formulations are registered in compliance with FDA under the Modernization of Cosmetics Regulation Act of 2022.

The product described is not a drug product and was initially used solely to improve the condition and appearance of the skin. For all cell-based assays and animal studies, heat shocked and non-heat shocked ASC-derived exosomes were diluted in PBS and PBS alone was used as a vehicle control. For the patient case study, ASC-derived exosomes were blended with a base formulation consisting of 1.0% hyaluronic acid-based gel formulation and used as an over the counter (OTC) personal care product.

### Diabetic Wound Healing in Patient Volunteer A Case Study

The presented case involves a 55-year-old male patient with a 30-year diagnosis of diabetes mellitus type 2 with stable A1C and other investigations within normal limits, presenting with multiple Grade 1 non-healing ulcers over the foot that had been previously treated for over 6 months by a foot surgeon. The surgeon had not been successful in healing these wounds using conventional therapy including debridement, off-loading with a soft cast, infection management and weekly dressing changes.

Over the prior 6-month treatment period to promote healing, various dressing types were tried including alginate, silver, hydrocolloid and honey impregnated dressings. Despite active treatment, 3 large ulcers remained on the foot and one on the toe. Due to the patient's history of chronic non-healing foot ulcers, an alternate solution was sought to accelerate healing.

The patient volunteer was treated once weekly with topical application of tuned ASC-derived exosome test article containing about 1×10E9-5×10E9 exosomes. Ulcers were thoroughly cleaned but not debrided. Exosome gel was applied topically directly to the wound bed, sufficiently to cover ulcers followed by Tegaderm® (i.e., a transparent, sterile, waterproof film dressing) to cover the gel as well as a secondary dressing to cushion and protect the treatment areas. The wounds were monitored regularly for signs of infection or complications but treatment and dressing were only changed and reapplied weekly. Foot wounds were photographed by care provider and ImageJ® was used to detect open wound and quantify pixel area.

### Statistical Analysis

Quantitative data were expressed as mean±standard deviation (SD) based on at least three independent assays in duplicates or triplicates. Students unpaired t-tests were performed using GraphPad® Prism (GraphPad®) for all in vitro assays. ANOVA was used to determine significance in the described animal wound healing study as well as in the human case study. Graphs are presented mean±SD or mean±s.e.m. All p-values below 0.05 (*), 0.01 (**), and 0.001 (***) were considered statistically significant.

### Results

### Extracellular Vesicles Characterization

The manufacturing process using ASC-derived EVs was adapted from previously published protocols (Basu and Ludlow, 2016). To ensure lot consistency, MISEV guidelines for EV characterization were followed using multiple methods for identification (Zhang et al., 2024; Welsh et al., 2024). Each lot was screened using immunofluorescence to identify the presence of exosomes surface markers (see, FIG. 31).

Purified particles were stained with antibodies targeting tetraspanin proteins CD9, CD63, and CD81. Positive identification was confirmed while substantially negative expression of lymphocytic markers CD3, CD4, and CD19 illustrated that the preparation was substantially pure (see, FIG. 31). Particle size was determined using ZetaView® NTA analysis showing a distribution range from 65 nm to 200 nm (FIG. 29), consistent with published exosome biology (Gurunathan et al., 2021; Zhang et al., 2020). Tuned ASC-derived exosomes have a median particle size of 129±4 nm ranging from 65 nm to 200 nm. TEM was used to validate vesicle size and morphology, confirming isolation and purification of EVs with bi-membrane vesicles sized at 80-140 nm in diameter (see, FIGS. 30A-30B).

miRNA Cargo Characterization

Full protein characterization and miRNA sequencing of tuned ASC-derived exosomes was conducted via RNA-Seq (as described in Example 3), revealing the full transcriptome of tuned ASC-derived exosomes. The miRNA cargo of tuned ASC-derived exosomes was characterized and categorized according to functional relevance in wound healing applications.

A wound is considered to be chronic if it hasn't started to heal after 4 to 12 weeks despite treatment. The dysregulation of miRNA in chronic wounds has highlighted the opportunity for new targets of therapeutic intervention. Replenishing specific miRNA levels which may be deficient could potentially improve wound healing in chronic conditions.

In the context of wound healing, specifically chronic wounds, decreased levels of specific miRNAs, like miR-29, miR-143, miR-10a, miR-146a, and miR-199 are often observed in lesional skin. Increased inflammation is also characteristic of this condition. Low expression of these miRNAs can exacerbate symptoms.

Table 34 below presents miRNAs identified in ASC-derived exosomes with significance to wound healing.

TABLE 34
miRNAs with Significance to Wound Healing

| miRNA | Significance/Function |
|---|---|
| miR-143, miR-29a, miR-140 | Collagen Production |
| miR-29a, miR-146a | Cell Proliferation |
| miR-10a, miR-21, miR-146a, miR-199, miR-143, | Immune Regulation |
| miR-140 | |
| miR-10a, miR-21, miR-146 | Wound Healing |
| miR-143, miR-21, miR-199, miR-155 | Angiogenesis |

### ASC-derived Exosomes Stimulate Collagen Production and Reduce Inflammation

Chronic wound tissue is associated with degradation of the extracellular matrix and collagen fiber degeneration. The first stages of wound healing require collagen deposition to support extracellular matrix formation which facilitates cell migration and tissue regeneration.

### ASC-derived Exosomes Stimulate Cell Migration and Angiogenesis

When cells are seeded in the well of the Radius® plate, cells adhere everywhere except in the center of the well where there is a biocompatible hydrogel spot of a defined and constant area. Once seeded cells form a monolayer, the assay is initiated by gently dissolving the gel with a removal solution, leaving a gap across which cell migration can occur.

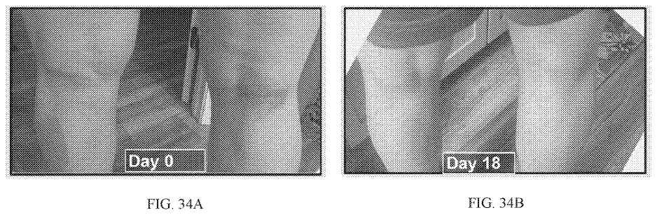

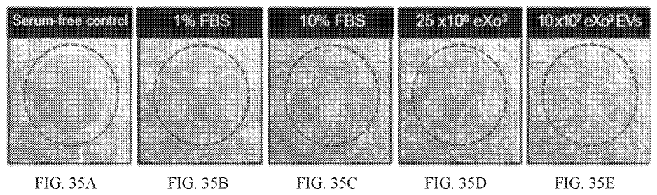

FIG. 35A, FIG. 35B, FIG. 35C, FIG. 35D, and FIG. 35E shows results of a Radius® plate cell migration assay using human primary skin epithelial cells, seeded at 100,000 cells/well, in which a biocompatible Radius™ hydrogel creates a circular area across which seeded cells may migrate following gel removal. FIG. 35A shows that at T=48 hours, serum free media (negative control) showed minimal migration. FIG. 35B shows that 1% FBS media exhibits slightly more migration. FIG. 35C shows that 10% FBS media showed full closure of the gap. FIG. 35D shows the addition of 25×10E6 tuned ASC-derived exosomes (eXo3) to the 1% FBS media, which revealed slightly better gap closure compared to the 1% FBS media alone (FIG. 35B). FIG. 35E shows that 10×10E7 tuned ASC-derived exosomes (eXo3) elicited complete gap closure when added to the 1% FBS media.

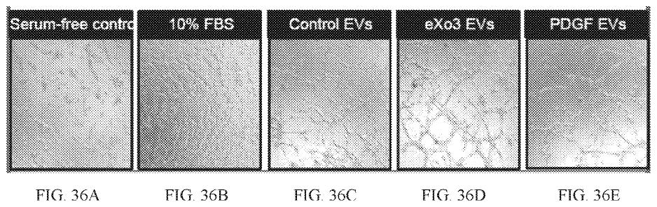

FIG. 36A, FIG. 36B, FIG. 36C, FIG. 36D, and FIG. 36E show enhanced vascular networks with tuned ASC-derived exosome (eXo3) treatment of human umbilical vein endothelial cells (HUVECs) when compared to a serum free treatment control, a 10% FBS treatment control, and a platelet-derived growth factor treatment control. FIG. 36A shows HUVECs seeded in Matrigel and treated with a serum free non-heat shocked exosome treatment control. FIG. 36B shows HUVECs seeded in Matrigel and treated with a 10% FBS treatment control. FIG. 36C shows HUVECs seeded in Matrigel and treated with control (non-heat shocked) EVs. FIG. 36D shows HUVECs seeded in Matrigel and treated with tuned ASC-derived exosomes (eXo3). FIG. 36E shows HUVECs seeded in Matrigel and treated with a platelet-derived growth factor (PDGF) treatment control.

### ASC-Derived Exosomes Improve Recovery in Rodent Wound Healing Study

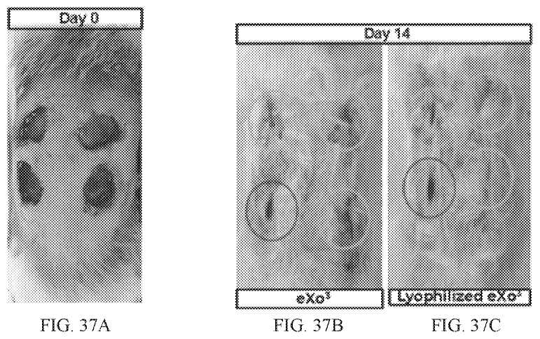

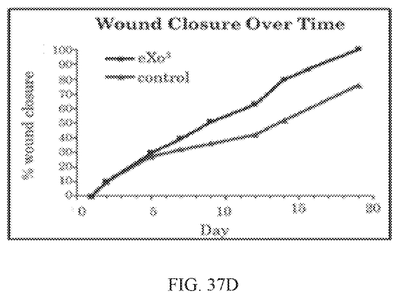

A rat excisional wound model was used in which 2 cm diameter wounds were created in the dorsal skin of Sprague-Dawley rats (see, FIG. 37A); rats were treated topically with a single dose of vehicle (300 μL) or tuned ASC-derived exosomes (25×10E6 particles). The wounds were measured every 2-3 days to monitor the healing process.

FIG. 37A, FIG. 37B, FIG. 37C, and FIG. 37D shows wound healing using a rat model. FIG. 36A shows four excision wounds made on the animal at day 0. FIG. 37B shows day 14 of treatment with either vehicle or tuned ASC-derived exosomes (eXo3). Bottom left circles represent vehicle control. FIG. 37C shows day 14 of treatment with either vehicle or lyophilized tuned ASC-derived exosomes (lyophilized eXo3). Bottom left circles represent vehicle control. FIG. 37D is a plot of % wound closure (y-axis) versus time in days over a 20 day period. ASC-derived exosome treatment shows enhanced wound closure. ANOVA p<0.05.

The wound closure rate was increased by tuned ASC-derived exosome treatment after 14 days compared to the vehicle control (see, FIG. 37B, eXo3). Tuned ASC-derived exosome subjected to lyophilization and stored at room temperature for 7 days prior to reconstitution maintained their capacity to improve wound healing in vivo (see, FIG. 37C, Lyophilized eXo3). FIG. 37D is a graphic representation of the closure rates (y axis) over time (days, x-axis) Lyophilized tuned ASC-derived exosomes, when reconstituted with sterile water, resulted in a wound closure rate comparable to that of freshly-isolated EVs (data not shown).

Functional studies demonstrated that tuned ASC-derived exosomes stimulate collagen production and cell proliferation. In histological sections showing collagen deposition in wounds, tuned ASC-derived exosome treatment demonstrated increased collagen fiber organization and density compared to saline treatment and native exosomes using Verhoeff-Van Gieson (EVG) staining (i.e., a histological technique used to differentiate elastic fibers from collagen fibers).

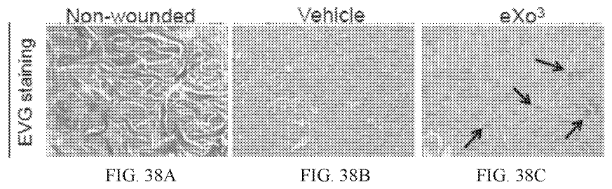

FIG. 38A, FIG. 38B, and FIG. 38C show tissue sections stained with Verhoeff-Van Gieson (EVG stain) to identify changes in collagen fibers in rat excisional wound sites 8 days after injury. The tissue is stained with a regressive hematoxylin, consisting of ferric chloride and iodine. The differentiating is accomplished by using excess mordant (ferric chloride) to break the tissue-mordant dye complex. FIG. 38A shows collagen production in a non-wounded rat. FIG. 38B shows collagen production in a vehicle-treated wound. FIG. 38C shows increased collagen production in a wound treated with tuned ASC-derived exosomes (eXo3) (arrows).

Greater collagen deposition and organization was observed by EVG staining in the tuned ASC-derived exosomes treated wounds, supporting a more rapid and better rate of healing than vehicle treatment.

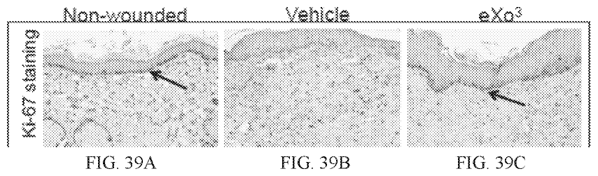

FIG. 39A, FIG. 39B, and FIG. 39C shows results of staining rat excisional wound sites 8 days after injury to detect Ki-67, a nuclear marker for proliferating cells. Ki67 is present during all active phases of the cell cycle (G1, S, G2, and M) but is absent in resting cells (G0). FIG. 39A shows Ki-67 staining in a non-wounded rat. FIG. 39B shows Ki-67 staining in a vehicle-treated wound. FIG. 39C shows increased Ki-67 staining in a wound treated with tuned ASC-derived exosomes (eXo3).

Immunohistochemical staining for Ki-67 in wound tissue in rats demonstrated increased Ki-67 positive cells in tuned ASC-derived exosome-treated samples compared to vehicle-treated wounds. tuned ASC-derived exosome-treated wound tissue demonstrated darker staining for Ki-67 than vehicle, suggesting that there is more proliferation in tuned ASC-derived exosomes-treated wounds.

### Case Study: Topical Application of Tuned ASC-Derived Exosomes Promotes the Appearance of Diabetic Ulcer Healing

Given that tuned ASC-derived exosomes significantly enhanced wound healing in the rodent model, the active ingredient was formulated for human skin application. The topical ASC-derived exosomes formulated in a hyaluronic acid gel was initially used solely to improve the condition and appearance of the skin. However, when applied to subjects suffering from diabetic ulcers which were previously refractive to currently accepted and practiced methods of treatment, dramatic effects were observed.

A human volunteer case study was documented where tuned ASC-derived exosomes gel was applied topically directly to the wound bed (see, FIG. 40A). The four ulcers were sufficiently covered with tuned ASC-derived exosomes gel followed by Tegaderm to cover the gel as well as a secondary dressing to cushion and protect the treatment areas. The wounds were monitored regularly for signs of infection or complications but treatment and dressing were only changed and reapplied weekly. Wound healing was documented after each dressing change.

FIG. 40A, FIG. 40B, FIG. 40C, and FIG. 40D show photographs of human volunteer single case study observations after topical application of tuned ASC-derived exosomes to chronic diabetic ulcers. FIG. 40A shows the diabetic ulcers before treatment. FIG. 40B shows the diabetic ulcers 30 days after treatment. FIG. 40C shows the diabetic ulcers 60 days after treatment. FIG. 40D shows the diabetic ulcers 120 days after treatment, indicating improvement over the course of the treatment.

After 4 weeks of treatment, three lesions were completely healed, while the largest lesion showed >75% recovery (see, FIG. 40B). Treatment continued for 3 more weeks and resulted in nearly 100% recovery (see, FIG. 40C). Photographic imaging documented the healing of the lesion over the course of 120 days with weekly topical treatment (see, FIG. 40D). Quantitative image analysis of identified lesions in images identified open wound area and calculated the lesion size. Each wound site was normalized to baseline area at time 0 and the percent closure was calculated throughout the term of collection.

These findings support the ability to “tune” pro-healing EVs that provide therapeutic benefit beyond traditional pharmacological treatments.

### Case Study: Topical Application of Tuned ASC-Derived Exosomes Promotes Post Surgical Recovery

A subject with a history of keloid formation was treated with topical application of tuned ASC-derived exosomes, post-surgery. Photographic documentation was obtained before treatment and after application of the tuned ASC-derived exosome formulation.

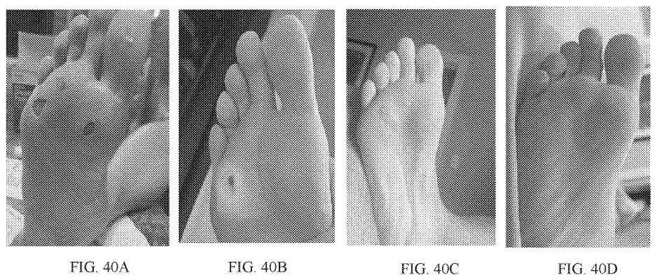

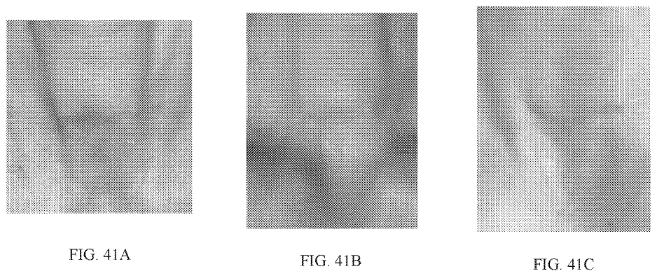

FIG. 41A, FIG. 41B, and FIG. 41C show photographs of post-surgery scars of a subject with a history of keloid formation after topical application of tuned ASC-derived exosomes (quantity) indicating improvement over the course of the treatment. FIG. 41A shows the scars before treatment. FIG. 41B shows the scars during the treatment with ASC-derived exosomes on day 14. FIG. 41C shows the scars after 21 days of treatment. Significant improvement in the surgery scar was observed after treatment

### Case Study: Topical Application of Tuned ASC-Derived Exosomes Promotes Improvement in Lip Ulceration

A subject with a lip ulceration was treated by topical application of tuned ASC-derived exosomes. Photographic documentation was obtained before treatment and 16 hours after application of the tuned ASC-derived exosome formulation.

Significant improvement in the lip ulceration was observed within 16 hours after treatment (see, FIGS. 42A-42B).

FIG. 42A and FIG. 42B show photographs of a subject's lip with a lip ulceration. FIG. 42A shows the lip ulceration before treatment with tuned ASC-derived exosomes. FIG. 42B shows the lip ulceration 16 hours after the treatment with tuned ASC-derived exosomes, indicating improvement over the course of the treatment.

### Case Study: Topical Application of Tuned ASC-Derived Exosomes Promotes Improvement in Gum Recession

A subject presenting with gum recession was treated twice daily by application of a biomaterial wound matrix (Mirragen® Advanced Wound Matrix) combined with tuned ASC-derived exosomes. Photographic documentation was obtained at baseline showing the receding gum tissue and at 7 days post treatment.

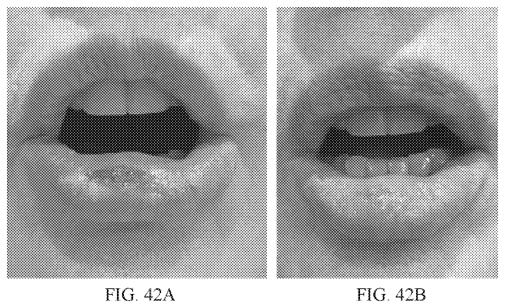

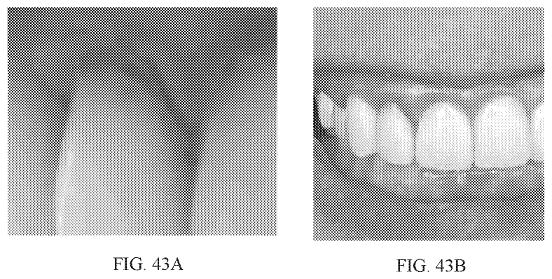

FIG. 43A and FIG. 43B show photographs of a subject's gum recession. FIG. 43A shows the gum recession before treatment with tuned ASC-derived exosomes. FIG. 43B shows the gum recession 7 days after the treatment with tuned ASC-derived exosomes twice daily, indicating improvement over the course of the treatment. Significant improvement in gum recession was observed at 7 days post twice daily application of the biomaterial wound matrix combined with tuned ASC derived exosomes.

### Discussion

Stem cell therapies represent a compelling means of tissue repair and have demonstrated efficacy in wound healing and soft tissue regeneration in animal models. Stem cell therapeutic approaches have the advantage of being capable of potentially regenerating an assortment of tissues by virtue of their pluripotency. Such versatility is a much-needed option for various medical conditions. The downside of stem cell therapy includes their complexity in isolation, characterization, and patient delivery. The greatest concern continues to be the potential for uncontrolled cell growth, proliferation, and tumorigenesis. The use of embryonic stem cells for therapeutic applications also raises ethical concerns and continues to be a passionately debated topic. Additionally, the intuitive concept that therapeutic stem cells engraft and differentiate at sites of tissue damage is not well supported given the low numbers of cells retained over time at application sites in vivo (Lotfy et al., 2023). This suggests that their mechanisms of action occur through paracrine modalities such as secretion of bioactive vesicles, including exosomes. Hence, exploiting ASC-derived exosomes as a biologic-derived therapy, rather than as delivering transient stem cells to treat chronic wounds, is an enticing approach.

Secreted extracellular vesicles (EVs), such as exosomes, are packed with potent pro-repair proteins and RNA cargos that are both cell type-specific, as well as, differentially produced and secreted according to the cellular environment (Zhang et al., 2019; Kim et al., 2017; Qiu et al., 2020). Exosomes offer a novel approach to tissue engineering and regenerative medicine therapies and have several advantages over the use of cells. Since exosomes are cell-free, there is a much lower risk of immune rejection, which argues for their allogenic use in the clinic. Exosome regenerative and wound healing capabilities are also heightened by having an elevated concentration of growth factors compared to cells. Furthermore, manufacturing large-scale, GMP-grade product is accomplished at sustainably lower costs.

The data presented herein supports the concept that manipulation of the cellular bioreactor environment improves the repair activity of ASC-derived exosomes in vitro and in vivo. Current investigation into the cell-signaling elicited by these ASC-derived exosomes will facilitate further understanding of the mechanisms involved in wound healing and tissue repair. Towards this goal, the miRNA cargo contained in ASC-derived exosomes has been partially analyzed and several sequences implicated in wound healing have been identified. Additionally, current proteomic research has identified cell cycle regulatory proteins and stress response proteins such as HSP70 that may contribute to the observed pro-healing function of ASC-derived exosomes. Once the analysis is complete, the miRNA and protein cargos believed to be important for this healing effect will be reported and may potentially lead to additional tissue engineering and regenerative medicine applications.

The single human volunteer case study reported herein shows promising therapeutic benefit and wound healing potential of ASC-derived exosomes application. Diabetic foot ulcers remain a therapeutic area of high medical unmet need. The preclinical data presented, including the animal studies, combined with the morbidity and mortality associated with diabetic foot ulcers served to highlight the critical need to treat this patient. Given that 70% of diabetic foot ulcers remain unhealed at 20 weeks, the rapid closure of these wounds is significant.

The rationale for why this patient was treated as well as the successful healing presented will hopefully serve as justification for larger clinical trials in this area of unmet need. Follow-up studies over time are required to adequately assess the effectiveness of ASC-derived exosomes in its current product formulation. With a growing number of studies focused on wound healing, the data presented and research efforts described herein provide valuable translational evidence supporting the therapeutic benefits of exosomes (Hu et al., 2016; Qiu et al., 2020; Shabbir et al., 2015; Shi et al., 2021).

### Example 7: Clinical Evaluation of Topical Exosome Compositions for Hair Growth and Scalp Health

### Rationale

Tuned exosomes derived from ASCs and Aloe vera plants can be formulated for topical application to address hair loss and improve scalp conditions. The tuned ASC-derived and Aloe-derived exosomes contain miRNA cargo that can influence hair follicle development, dermal papillae cell proliferation, and the hair growth cycle. miRNAs are considered strong gene regulators as they control gene expression and cell signaling pathways. miRNAs play a role in hair growth regulation and are involved in hair follicle development as well as in dermal papillae cell proliferation. miRNA signatures may be utilized to target key pathways in the hair growth cycle, including activating and accelerating dermal papilla (DP) cell proliferation and follicle development, as well as modulating the Wnt/0-catenin signaling pathway.

Dysregulation of the Wnt/0-catenin signaling pathway is associated with the development of androgenetic alopecia (AGA), and dihydrotestosterone (DHT) induces secretion of DKK1, a protein that inhibits the Wnt/0-catenin signaling pathway, resulting in β-catenin degradation and keratinocyte death (Papukashvili, D. et al., Cells (2021), 10, 2957). Reactivation of the Wnt/0-catenin signaling pathway in subjects with alopecia has emerged as a target to address AGA. There are miRNAs that target DKK1 and inhibit DHT.

The tuned ASC-derived exosomes of the present application are specifically tuned to enhance exosomal cargo and bioactivity to provide scalp health benefits and create a healthy environment for hair follicles. The tuned ASC-derived exosomes cargo transcriptome has been characterized with defined miRNA and protein content.

Alone and in combination with tuned Aloe-derived exosomes, tuned ASC-derived exosomes have demonstrated functional activity in dermal papillae (DP) cell proliferation and activation models. In preclinical studies, the donor proteins and miRNA in tuned ASC-derived exosomes have been shown to influence cell signaling, inflammatory response, collagen, elastin and hyaluronic acid production, oxidative stress response, response to UV damage and photoaging, follicle condition and health, and the hair growth cycle.

miRNA Cargo Characterization

The miRNA cargo of tuned ASC-derived exosomes was characterized and categorized according to functional relevance in hair growth applications. Table 35 below presents miRNAs identified in tuned ASC-derived exosomes with significance to hair growth and inhibition of hair loss.

TABLE 35
miRNAs Associated with Hair Growth and Inhibition of Hair Loss

| miRNA | Significance |
|---|---|
| miR-21-5p | Regulates hair follicle development CNKSR2 KLF3 TNPO1 |
| miR-24 | Increases hair keratinocyte differentiation |
| miR-26a-5p | Regulates hair follicle development and growth TGF/β-catenin |
| miR-146a | Targets and inhibits DKK1 |
| let-7b | Regulates hair growth via targeting EDA |
| miR-101-3p | Promotes proliferation and inhibits apoptosis of hair follicle stem cells by |
| | targeting DUSP1; also targets and inhibits DKK1 |
| miR-181a-5p | Promotes hair growth |
| miR-21-3p | Regulates hair follicle development CNKSR2 KLF3 TNPO1 |
| miR-148b-3p | Promotes proliferation of hair follicle stem cells NFAT5 |
| miR-486-5p | Maintains anagen phase; prevents immune privilege collapse of follicles in |
| | alopecia areata |
| miR-103a-3p | Targets and decreases DKK1 |

### Hair Composition Formulations

Two topical hair formulations were prepared.

The EXO HC-001 formulation is a serum comprising both tuned ASC-derived exosomes and tunedAloe-derived exosomes in combination with growth factors (ANARGY™ EPITENSIVE®, and keratinocyte growth factor (KGF), niacinamide, copper tripeptide-1, saw palmetto, biotin, panthenol, silkgel NEG®, and hyaluronic acid.

The EXO HC-002 formulation is a spray comprising tunedAloe-derived exosomes in combination with niacinamide, copper tripeptide-1, saw palmetto, biotin, panthenol, silkgel NEO®, hyaluronic acid, cationic conditioners, AnaGain™, Hairgenyl®, antifungal agent, safflower oil, and fragrance. The main ingredients and their inclusion in each formulation are presented in Table 36 below.

TABLE 36
EXO Hair Products - Main Ingredients

| | EXO | | EXO | |
|---|---|---|---|---|
| Main Ingredients | HC-001 | Concentration | HC-002 | Concentration |
| Tuned ASC-derived | X | | | |
| Exosomes | | | | |
| Tuned Aloe-derived | X | | X | |
| Exosomes | | | | |
| Niacinamide | X | 4.0% | X | 4.0% |
| Growth Factors | X | | | |
| (ANARGY ™ | | | | |
| EPITENSIVE ®, | | | | |
| KGF) | | | | |
| Copper Tripeptide-1 | X | 0.000001-4% | X | 0.000001-4% |
| Saw Palmetto | X | 0.000001-4% | X | 0.000001-4% |
| Biotin | X | 0.000001-4% | X | 0.000001-4% |
| Panthenol | X | 0.000001-4% | X | 0.000001-4% |
| Silkgel NEO ® | X | 0.000001-4% | X | 0.000001-4% |
| Hyaluronic Acid | X | 0.000001-4% | X | 0.000001-4% |
| Cationic | | | X | |
| Conditioners | | | | |
| ANAGAIN ™ | | | X | |
| HAIRGENYl ® | | | X | |
| Antifungal | | | X | |
| (Echinacea) | | | | |
| Safflower Oil | | | X | |
| Fragrance | | | X | |

Growth factors in the EXO HC-001 formulation include: (1) ANARGY® (IGF-1, VEGF, and sh-polypeptide-86) at 2.5% concentration; (2) EPITENSIVE® (EGF) at 2.0% concentration; and (3) KGF at 2% concentration.

An additional hair formulation, designated EXO HC-003, was prepared comprising both tuned ASC-derived exosomes and tuned Aloe-derived exosomes at the highest dual concentration. The EXO HC-003 formulation is a comprehensive hair treatment composition that combines the tuned exosomes with cationic conditioners, biomimetic peptides, hair essential micro- and macro-nutrients, and additional clinically proven active ingredients. The complete formulation is presented in Table 37 below.

TABLE 37
EXO HC-003 Hair Formulation

| Ingredient Name | Ingredient % Range |
|---|---|
| Water | 50-95 |
| Niacinamide | 0.000001-4 |
| Polyglyceryl-3 Betainate Acetate | 0.000001-4 |
| Behentrimonium Chloride | 0.000001-4 |
| Isopropyl Alcohol | |
| Cetearyl Alcohol | 0.000001-4 |
| Amodimethicone | 0.000001-4 |
| Trideceth-12 | |
| Cetrimonium Chloride | |
| Isomalt | 0.000001-4 |
| Water | |
| Pisum Sativum (Pea) Sprout Extract | |
| Water | 0.000001-4 |
| Glycerin | |
| Copper Tripeptide-1 | |
| Caprylyl Glycol | |
| Ethylhexylglycerin | |
| Isopentyldiol | 0.000001-4 |
| Glycereth-7 Triacetate | 0.000001-4 |
| Tuned Human ASC-derived Exosome, | 0.000001-4 |
| Aloe Vera Leaf Extract | |
| Phenoxyethanol | 0.000001-4 |
| Panthenol | 0.000001-4 |
| Phenyl Trimethicone | 0.000001-4 |
| Serenoa Serrulata Fruit Extract | 0.000001-4 |
| Fragrance | 0.000001-4 |
| Yeast Extract | 0.000001-4 |
| Maltodextrin | |
| Water | |
| Potassium Sorbate | |
| Sodium Benzoate | 0.000001-4 |
| Citric Acid | 0.000001- |
| Water | 0.000001-4 |
| Silk | |
| 1,2-Hexanediol | |
| Caprylyl Glycol | |
| Carthamus Tinctorius (Safflower) Seed Oil | 0.000001-4 |
| Echinacea Purpurea Extract | |
| Xanthan Gum | 0.000001-4 |
| Sodium Hyaluronate | 0.000001-4 |
| Biotin | 0.000001-4 |

Niacinamide at 4% concentration improves scalp health, improves circulation in the scalp, provides antioxidant activity, hydrates the scalp and hair, strengthens hair through stimulating protein synthesis, reduces inflammation, regulates scalp and hair oil production, supports hair strength, and reduces hair loss.

Copper tripeptide-1 accelerates the rate of hair growth, improves blood flow to hair follicles, reduces hair loss, prevents hair thinning, regenerates damage to the scalp, and inhibits dihydrotestosterone (DHT).

Saw palmetto at 0.5% promotes scalp health, prevents hair loss by potentially preventing the body from converting testosterone into DHT, promotes hair growth, strengthens the hair follicle, and reduces inflammation.

AnaGain™, based on organic pea sprouts, stimulates specific signal molecules in the dermal papilla required to reactivate hair growth. AnaGain™ upregulates Noggin and FGF-7, which play a role in hair growth and follicle health. The high content of L-arginine supports natural hair growth, as this amino acid is known for its importance in the microcirculation in the dermal papilla cells. In a clinical study involving AnaGain™, the anagen:telogen coefficient (proportion of active hair follicles to degenerating ones) was improved from 4 to 7.2, corresponding to a 78% increase in hair growth in 3 months. AnaGain™ powder was used at 2% concentration.

Hairgenyl® is obtained from the yeast Pichia minuta, isolated from the flowers of azalea Rhododendron indicum, and is rich in biopeptides with low molecular weight produced through a non-denaturing production process. At the heart of hair follicle biology, Hairgenyl® targets three regulators of hair growth. Increasing mitochondrial fusion boosts the capacity for energy production that is used for growth of the hair follicle. Hairgenyl® also corrects the abnormal expression of signaling molecules and epigenetics involved in androgenetic alopecia. Hairgenyl® was used at 0.3% concentration.

In 2D and 3D cell and tissue models mimicking androgenetic alopecia, Hairgenyl® demonstrated efficacy on the dermal papilla.

Hairgenyl® works by supporting the biological activity of the dermal papilla and stimulating hair follicle growth. Hairgenyl® targets dermal papillae cells, influences the hair growth cycle, increases hair density and thickness, and reduces hair loss. Tested over 8 months on a group of male volunteers, Hairgenyl® increased hair density (+24.9%), and the majority of volunteers (85%) considered their hair became thicker (see, FIG. 44A, FIG. 44B).

FIG. 44A and FIG. 44B shows an increase in hair density in a male subject after topical application of Hairgenyl® a peptide-rich ingredient from the yeast Pichia minuta, isolated from the flowers of azalea (Silab) indicating improvement over the course of the treatment. FIG. 44A shows the subject on day 0 before application; FIG. 44B shows the subject after 8 months of application.

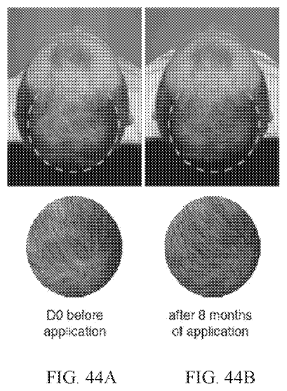

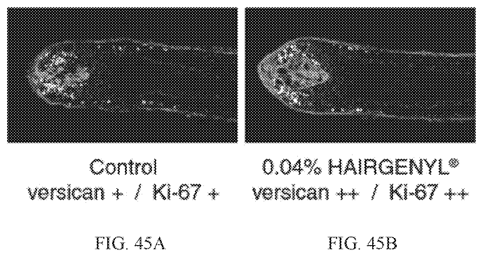

FIG. 45A and FIG. 45B shows Ki-67 and versican staining of a control hair follicle. Versican is a large ECM proteoglycan that plays important roles in cell-matrix interactions during adhesion, migration and inflammatory responses. FIG. 45A shows versican and Ki-67 staining of a hair follicle treated with a control. FIG. 45B shows enhanced versican and Ki-67 staining of a hair follicle treated with 0.04% Hairgenyl®.

Research also indicates that Hairgenyl® works by acting on the exosomal communication system between the dermis and the hair follicle, further supporting hair growth and potentially enhancing the utility of Aloe-derived exosomes.

Echinacea purpurea extract is included in the EXO HC-002 formulation as an anti-fungal agent. Echinacea has anti-inflammatory properties that may help to soothe an itchy, irritated scalp, provides anti-fungal activity, and has the ability to increase blood flow which may help to improve hair growth.

Safflower oil (Carthamus tinctorius seed oil) is included in the EXO HC-002 formulation. Safflower oil is rich in polyunsaturated fats, containing 77% linoleic acid (omega-6 essential fatty acids), 10% saturated fats, and 13% oleic acid (omega-9 fats). Safflower oil also comprises high levels of vitamin E, alpha-tocopherol, vitamin K, and phytosterols. The oil contains high amounts of oleic acid, which is light in texture and penetrates the skin easily, and may protect, nourish, hydrate, and firm the hair, as well as promote hair growth and thickness. Being rich in essential polyunsaturated fatty acids, safflower oil may be beneficial for dry, color treated, or permed hair.

When applied to the scalp, safflower oil may act as a vasodilator which can dilate the blood vessels, resulting in increased nutrient supply to the hair follicles through blood circulation and a healthy environment for hair regrowth. Hair normally contains a moisture content of approximately 10%, and as the hair grows past the neckline or does not have direct contact with the scalp, the essential natural oils present in the scalp are not utilized throughout the hair shaft. Moisturizers that are rich in essential fatty acids (EFA), such as safflower oil, may increase the hair's ability to attract and retain moisture in the cortex of the hair.

### Clinical Case Reports

Clinical case reports were documented through photographic evidence to assess the effects of topical application of the exosome-containing hair formulations on various types of hair loss conditions.

### Male Pattern Hair Loss

In a first case report, a subject with male pattern hair loss was treated with the EXO Hair formulations HC-002 and HC-003. Photographic documentation was obtained before treatment and after 6 weeks of treatment.

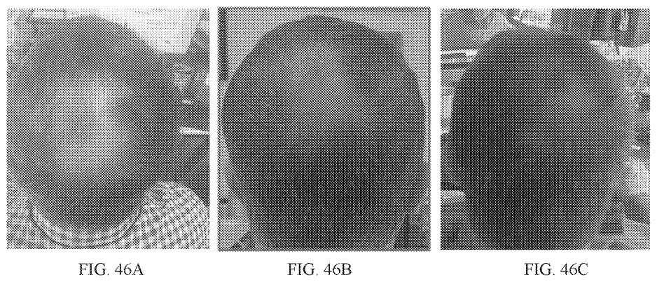

FIG. 46A, FIG. 46B, and FIG. 46C show photographs of a male subject with male pattern hair loss after topical application of tuned ASC-derived exosomes indicating improvement over the course of the treatment with EXO Hair formulations HC-002 and HC-003. FIG. 46A shows the hair before treatment. FIG. 46B shows the hair on day 45 of treatment. FIG. 46C shows the hair 9 weeks after treatment.

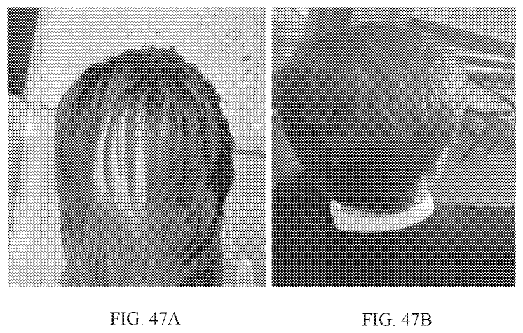

In a second case report, a subject with male pattern hair loss was treated with the EXO Hair formulation HC-001 and HC-002. Photographic documentation was obtained before treatment and after 12 weeks of treatment. Following treatment, the subject demonstrated visible improvement in hair coverage and density (see, FIGS. 47A-47B).

FIG. 47A and FIG. 47B show photographs of a subject with male pattern hair loss after topical application of tuned ASC-derived exosomes indicating improvement over the course of the treatment with EXO Hair formulation HC-001 and HC-002. FIG. 47A shows the hair before treatment. FIG. 47B shows the hair 12 weeks after treatment.

### Summary

The tuned ASC-derived exosomes and tuned Aloe-derived exosomes help signal the body's cells to reprogram behavior, restore function, and regenerate while additionally supporting the repair and rejuvenation of damaged tissue, including the scalp, hair follicles, and follicular cells. Without being limited by theory, the formulations activate the body's reparative processes to stimulate hair growth, promote hair health, and improve scalp condition.

The tuned ASC-derived exosomes and tuned Aloe-derived exosomes supports the entire hair growth cycle, not just surface-level symptoms or singular causes, by promoting and supporting natural healthy hair growth, activating and sustaining anagen, addressing factors that cause hair loss, reducing breakage and shedding, and considering both internal and external factors.

The tuned ASC-derived exosomes and tuned Aloe-derived exosomes formulations focus on hair health and hair longevity in a comprehensive approach that combines maintaining the health of the scalp and follicles, the hair growth cycle, and the hair fibers themselves. These EXO HC-001, HC-002, HC-003 formulations may offer a broad mechanism of action, delivering a complex cocktail of bioactive molecules that influence multiple signaling pathways simultaneously, including patented exosomes tuned to restore and enhance the power of the body's innate mechanisms of repair and regeneration, biomimetic peptides, hair essential micro and macro nutrients, cationic conditioners, a protein rich matrix to create structural support, and additional clinically proven active ingredients including niacinamide, copper tripeptide, AnaGain™, Hairgenyl®, Anargy™, biotin, and saw palmetto. The formulations may foster complete hair health from root to tip by hydrating, nourishing, reprogramming, protecting, strengthening, and revitalizing the hair and scalp.

### Example 8: Topical Exosome Compositions for Intimate Care

### Rationale

Exosome therapy offers a comprehensive solution for women experiencing tissue changes due to childbirth, menopause, or aging by promoting natural tissue regeneration, improved elasticity, enhanced sensation, and restored confidence through a non-surgical, biocompatible approach. Tuned ASC-derived exosomes and tuned Aloe-derived exosomes promote cell proliferation and migration, helping regenerate vaginal tissues including the epithelium and connective tissue The regenerative approach of exosome-based intimate care compositions may provide multiple therapeutic advantages.

### Tissue Regeneration and Renewal

Tuned ASC-derived exosomes and tuned Aloe-derived exosomes promote cell proliferation and migration, regenerating vaginal tissues including the epithelium and connective tissue. This may improve the overall structure and function of vaginal tissues, leading to increased tightness and sensation.

### Enhanced Collagen Synthesis

Exosomes contain growth factors and signaling molecules that can stimulate collagen synthesis in fibroblasts. Improved collagen production in the vaginal wall may enhance tissue elasticity and firmness, contributing to structural rejuvenation.

### Extracellular Matrix Remodeling

Tuned ASC-derived exosomes and tuned Aloe-derived exosomes regulate the synthesis and degradation of extracellular matrix (ECM) proteins such as collagen and elastin. Maintaining the balance of these proteins may improve the structural support and hydration of vaginal tissues.

### Angiogenesis and Improved Blood Flow

Exosomes promote the formation of new blood vessels by transferring pro-angiogenic factors such as VEGF to endothelial cells. Improved blood supply may enhance the delivery of oxygen and nutrients to vaginal tissues, promoting healing and rejuvenation.

### Anti-Inflammatory and Immunomodulatory Effects

Tuned ASC-derived exosomes and tuned Aloe-derived exosomes may suppress inflammation and modulate the immune response, promoting tissue repair and reducing potential side effects and complications associated with vaginal rejuvenation treatments.

### Antioxidant Protection

Exosomes protect cells from oxidative stress by transferring antioxidant molecules such as superoxide dismutase (SOD) and glutathione peroxidase. This may help maintain vaginal tissue health and reduce the signs of aging.

Tuned ASC-derived exosomes and tuned Aloe-derived exosomes were included in formulations designed to address vaginal dryness, atrophy, and discomfort; vaginal tightening; and clitoral stimulation and vaginal sensitization. The formulations are regenerative vaginal gels that combine tuned exosome biotechnology in a hyaluronic acid delivery system designed to harness the body's natural regenerative capabilities to promote tissue revitalization and restoration of the vaginal microenvironment.

The formulations include tuned Aloe-derived exosomes and/or ASC-derived exosomes along with clinically supportive nutrients and bioactive ingredients including Aloe vera extract, arginine, Ginkgo biloba, and Echinacea. These advanced regenerative vaginal treatments may revitalize vaginal tissue by promoting cellular renewal, improving tissue circulation, restoring elasticity, replenishing vaginal moisture, and supporting natural lubrication.

The formulation's advanced hyaluronic acid technology delivers seven different molecular weights of hyaluronic acid, providing comprehensive and lasting moisturization to all layers of the delicate vaginal skin, leaving vaginal tissue hydrated while strengthening the vaginal lining.

The formulations provide long lasting regenerative relief from vaginal dryness and discomfort, soothe irritation, and support overall vaginal health. The non-invasive topical treatments support vaginal health by addressing concerns that impact daily comfort, function, and intimacy without hormones, harsh chemicals, or discomfort, making the formulations suitable for women of all ages experiencing dryness, tissue laxity, or irritation.

### Formulations

Three intimate care gel formulations were prepared. The EXO Intimate Care Gel with Cooling formulation is a gel comprising tuned Aloe-derived exosomes in combination with hyaluronic acid components, allantoin, Aloe vera extract, arginine, dipotassium glycyrrhizate (licorice root), panthenol, Ginkgo biloba extract, and a cooling agent (menthyl ethylamido oxalate), formulated at pH 4.5-5.0. The complete formulation is presented in Table 38 below.

TABLE 38
EXO Intimate Care Gel with Cooling Formulation (pH 4.5-5.0)

| | | | | Individual |
|---|---|---|---|---|
| Trade Name | Ingredient Name | Trade % | Active % | Ingredient % |
| Water | Water | 80-95 | 100.00 | 80-95 |
| Zemea ® | Propanediol | 1.00-4.00 | 100.00% | 1.00-4.00 |
| Propanediol | | | | |
| USP Glycerin | Glycerin | 1.00-4.00 | 100.00% | 1.00-4.00 |
| Carbopol ® Ultrez ™ | Carbomer | 1.00-4.00 | 100.00% | 1.00-4.00 |
| 30 Polymer | | | | |
| Euxyl ™ PE 9010 | Phenoxyethanol | 1.00-4.00 | 88.5-91.5% | 0.885-0.915 |
| | Ethylhexylglycerin | | 8.5-11.5% | 0.085-0.115 |
| Hymagic ™ 4D | Water | 0.25-2.00 | up to 100% | 0.40-0.60 |
| | Sodium | | 0.2-0.5% | 0.001-0.003 |
| | Hyaluronate | | | |
| | Crosspolymer | | | |
| | Sodium | | 0.5-1.0% | 0.003-0.005 |
| | Hyaluronate | | | |
| | Sodium Acetylated | | 0.5-1.2% | 0.003-0.006 |
| | Hyaluronate | | | |
| | Hydrolyzed Sodium | | 0.1-0.2% | 0.0001-0.002 |
| | Hyaluronate | | | |
| | Pentylene Glycol | | 3-5% | 0.015-0.025 |
| | Ethylhexylglycerin | | 0.05-0.1% | 0.000-0.001 |
| BP - Triluronic | Sodium | 0.25-2.00 | 100.00% | 0.25-2.00 |
| Acid Powder | Hyaluronate | | | |
| Tuned Aloe-derived | Aloe Vera Leaf | 0.25-2.00 | 100.00% | 0.25-2.00 |
| Exosomes | Extract or | | | |
| | Aloe Vera | | | |
| | Extracellular | | | |
| | Vesicles | | | |
| Allantoin | Allantoin | 0.1-2.00 | 100.00% | 0.1-2.00 |
| Sodium Hydroxide | Sodium Hydroxide | 0.1-2.00 | 100.00% | 0.1-2.00 |
| Aloe Vera Gel 200:1 | Aloe Barbadensis | 0.01-1.00 | 100.00% | 0.01-1.00 |
| Conc. | Leaf Extract | | | |
| Arginine Base | Arginine | 0.01-1.00 | 100.00% | 0.01-1.00 |
| Ritamectant K2 | Dipotassium | 0.01-1.00 | 100.00% | 0.01-1.00 |
| | Glycyrrhizate | | | |
| D-Panthenol | Panthenol | 0.01-1.00 | 100.00% | 0.01-1.00 |
| DermoFeel ® PA-3 | Sodium Phytate | 0.01-1.00 | 100.00% | 0.01-1.00 |
| Frescolat ® X-Cool | Menthyl | 0.01-1.00 | 100.00% | 0.01-1.00 |
| | Ethylamido Oxalate | | | |
| Ginkgo Biloba | Glycerin | 0.01-1.00 | 40-60% | 0.04-0.06 |
| Extract in Glycerin | Water | | 40-60% | 0.04-0.06 |
| | Ginkgo Biloba Leaf | | 2-10% | 0.002-0.010 |
| | Extract | | | |
| D-3 100 IU/mg | Cholecalciferol | 0.00001-0.001 | 100.00% | 0.00001-0.001 |
| Vitamin E Acetate | Tocopheryl Acetate | 0.00001-0.001 | 100.00% | 0.00001-0.001 |
| Oil | | | | |
| Echinacea Purpurea | Echinacea Purpurea | 0.0000001-0.0001 | 100.00% | 0.0000001-0.0001 |
| | Extract | | | |
| Noni ™ Concentrate | Morinda Citrifolia | 0.0000001-0.0001 | 100.00% | 0.0000001-0.0001 |
| Liquid | Fruit Juice | | | |
| DermalRx ® KBGA | Water | 0.0000001-0.0001 | 44-46% | 0.00000044-0.00000046 |
| | Glycerin | | 44-46% | 0.00000044-0.00000046 |
| | Algae Extract | | 8-12% | 0.000000008-0.00000012 |
| MariMoist ® | Water | 0.0000001-0.0001 | 89-98% | 0.000000089-0.00000098 |
| R10041 | Chondrus Crispus | | 2-10% | 0.000000002-0.00000001 |
| | Extract | | | |
| | Sodium | | 0.01-0.1% | 0.0000000001-0.000000001 |
| | Hyaluronate | | | |
| Agascalm ™ | Propanediol | 0.0000001-0.0001 | 70-90% | 0.00000007-0.00000009 |
| | Glycerin | | 10-30% | 0.00000001-0.00000003 |
| | Agastache | | 0.5-1.5% | 0.000000005-0.000000015 |
| | Mexicana | | | |
| | Flower/Leaf/Stem | | | |
| | Extract | | | |
| Phytessence ™ | Glycerin | 0.0000001-0.0001 | 46-50% | 0.000000046-0.00000005 |
| Wakame | Water | | 46-50% | 0.000000046-0.00000005 |
| | Undaria Pinnatifida | | 5-10% | 0.00000005-0.0000001 |
| | Extract | | | |
| | Phenoxyethanol | | 1.00% | 0.0000000001-0.000001 |
| Mitostime ™ | Water | 0.0000001-0.0001 | up to 100% | 0.0000001-0.0001 |
| | Laminaria Digitata | | 5-10% | 0.00000005-0.000000010 |
| | Extract | | | |
| | Phenoxyethanol | | 0.5-2.00% | 0.00000001 |
| Ulva Lactuca | Water | 0.0000001-0.0001 | 73-74% | 0.000000073-0.000000074 |
| Organic Biogreen | Glycerin | | 23-24% | 0.000000023-0.000000024 |
| Extract | Ulva Lactuca (Sea | | 1.5-2.5% | 0.0000000015-0.000000025 |
| | Lettuce) Extract | | | |
| | Citric Acid | | 0.1-1% | 0.0000000001-0.00000001 |
| | Sodium Benzoate | | 0.27-0.33% | 0.0000000027-0.0000000033 |
| | Potassium Sorbate | | 0.13-0.17% | 0.0000000013-0.0000000017 |

The EXO Intimate Care Original Gel formulation is a gel comprising tuned Aloe-derived exosomes in combination with hyaluronic acid components, allantoin, Aloe vera extract, arginine, dipotassium glycyrrhizate (licorice root), panthenol, Ginkgo biloba extract, and Echinacea purpurea extract. The complete formulation is presented in Table 39 below.

TABLE 39
EXO Intimate Care Original Gel Formulation

| | | | | Individual |
|---|---|---|---|---|
| Trade Name | Ingredient Name | Trade % | Active % | Ingredient % |
| Water | Water | 80-95 | 100.00% | 80-95 |
| Zemea ® Propanediol | Propanediol | 1.00-4.00 | 100.00% | 1.00-4.00 |
| USP Glycerin | Glycerin | 1.00-4.00 | 100.00% | 1.00-4.00 |
| Carbopol ® Ultrez 30 | Carbomer | 1.00-4.00 | 100.00% | 1.00-4.00 |
| Polymer | | | | |
| Euxyl ™ PE 9010 | Phenoxyethanol | 0.25-2.00 | 88.5-91.5% | 0.885-0.915 |
| | Ethylhexylglycerin | | 8.5-11.5% | 0.085-0.115 |
| Hymagic ™ 4D | Water | 0.25-2.00 | up to 100% | 0.4-0.6 |
| | Sodium Hyaluronate | | 0.2-0.5% | 0.001-0.003 |
| | Crosspolymer | | | |
| | Sodium Hyaluronate | | 0.5-1.0% | 0.003-0.005 |
| | Sodium Acetylated | | 0.5-1.2% | 0.003-0.006 |
| | Hyaluronat | | | |
| | Hydrolyzed Sodium | | 0.1-0.2% | 0.0001-0.01 |
| | Hyaluronate | | | |
| | Pentylene Glycol | | 3-5% | 0.015-0.025 |
| | Ethylhexylglycerin | | 0.05-0.1% | 0.000-0.001 |
| BP - Triluronic Acid | Sodium Hyaluronate | 0.25-2.00 | 100.00% | 0.25-2.00 |
| Powder | | | | |
| Sodium Hydroxide | Sodium Hydroxide | 0.25-2.00 | 100.00% | 0.25-2.00 |
| Tuned Aloe-derived | Aloe Vera Leaf Extract | 0.25-2.00 | 100.00% | 0.25-2.00 |
| Exosomes | | | | |
| Allantoin | Allantoin | 0.25-2.00 | 100.00% | 0.25-2.00 |
| Aloe Vera Gel 200:1 | Aloe Barbadensis Leaf | 0.01-2.00 | 100.00% | 0.01-2.00 |
| Conc. | Extract | | | |
| Arginine Base | Arginine | 0.01-2.00 | 100.00% | 0.01-2.00 |
| Ginkgo Biloba Extract | Glycerin | 0.01-2.00 | 40-60% | 0.040-0.060 |
| in Glycerin | Water | | 40-60% | 0.040-0.060 |
| | Ginkgo Biloba Leaf | | 2-6% | 0.002-0.006 |
| | Extract | | | |
| Echinacea Purpurea | Echinacea Purpurea | 0.01-2.00 | 100.00% | 0.01-2.00 |
| | Extract | | | |
| Ritamectant K2 | Dipotassium | 0.01-2.00 | 100.00% | 0.01-2.00 |
| | Glycyrrhizate | | | |
| D-Panthenol | Panthenol | 0.01-2.00 | 100.00% | 0.01-2.00 |
| DermoFeel ® PA-3 | Sodium Phytate | 0.01-2.00 | 100.00% | 0.01-2.00 |
| D-3 100 IU/mg | Cholecalciferol | 0.00001-0.001 | 100.00% | 0.00001-0.001 |
| Vitamin E Acetate Oil | Tocopheryl Acetate | 0.00001-0.001 | 100.00% | 0.00001-0.001 |
| Noni ™ Concentrate | Morinda Citrifolia | 0.0000001-0.0001 | 100.00% | 0.0000001-0.0001 |
| Liquid | Fruit Juice | | | |
| Dermal Rx ® KBGA | Water | 0.0000001-0.0001 | 44-46% | 0.00000044-0.00000046 |
| | Glycerin | | 44-46% | 0.00000044-0.00000046 |
| | Algae Extract | | 8-12% | 0.000000008-0.00000012 |
| MariMoist ® R10041 | Wate | 0.0000001-0.0001 | 89-98% | 0.000000089-0.00000098 |
| | Chondrus Crispus | | 2-10% | 0.000000002-0.00000001 |
| | Extract | | | |
| | Sodium Hyaluronate | | 0.01-0.1% | 0.0000000001-0.000000001 |
| Agascalm ™ | Propanediol | 0.0000001-0.0001 | 70-90% | 0.00000007-0.00000009 |
| | Glycerin | | 10-30% | 0.00000001-0.00000003 |
| | Agastache Mexicana | | 0.5-1.5% | 0.000000005-0.000000015 |
| | Flower/Leaf/Stem | | | |
| | Extract | | | |
| Phytessence ™ | Glycerin | 0.0000001-0.0001 | 46-50% | 0.000000046-0.00000005 |
| Wakame | Water | | 46-50% | 0.000000046-0.00000005 |
| | Undaria Pinnatifida | | 5-10% | 0.00000005-0.0000001 |
| | Extract | | | |
| | Phenoxyethanol | | 0.5-2.00% | 0.0000000001-0.000001 |
| Mitostime ™ | Water | 0.0000001-0.0001 | up to 100% | 0.0000001-0.0001 |
| | Laminaria Digitata | | 5-10% | 0.00000005-0.00000001 |
| | Extract | | | |
| | Phenoxyethanol | | 0.5-2.00% | 0.0000000001-0.000001 |
| Ulva Lactuca Organic | Water | 0.0000001-0.0001 | 73-74% | 0.000000073-0.000000074 |
| Biogreen Extract | Glycerin | | 23-24% | 0.000000023-0.000000024 |
| | Ulva Lactuca (Sea | | 1.5-2.5% | 0.0000000015-0.000000025 |
| | Lettuce) Extract | | | |
| | Citric Acid | | 0.1-1% | 0.0000000001-0.00000001 |
| | Sodium Benzoate | | 0.27-0.33% | 0.0000000027-0.0000000033 |
| | Potassium Sorbate | | 0.13-0.17% | 0.0000000013-0.0000000017 |

The EXO Intimate Care Second Version Gel formulation is a gel comprising tuned Aloe-derived exosomes in combination with hyaluronic acid components, allantoin, aloe vera extract, arginine, dipotassium glycyrrhizate (licorice root), panthenol, and Ginkgo biloba extract, formulated at pH 4.5-5.0. The complete formulation is presented in Table 40 below.

TABLE 40
EXO Intimate Care Second Version Gel Formulation (pH 4.5-5.0)

| | | | | Individual |
|---|---|---|---|---|
| Trade Name | Ingredient Name | Trade % | Active % | Ingredient % |
| Water | Water | 80-95 | 100.00% | 80-95 |
| Zemea Propanediol | Propanediol | 1.00-4.00 | 100.00% | 1.00-4.00 |
| USP Glycerin | Glycerin | 1.00-4.00 | 100.00% | 1.00-4.00 |
| Carbopol Ultrez 30 | Carbomer | 1.00-4.00 | 100.00% | 1.00-4.00 |
| Polymer | | | | |
| Euxyl ™ PE 9010 | Phenoxyethanol | 0.5-2.00 | 88.5-91.5% | 0.885-0.915 |
| | Ethylhexylglycerin | | 8.5-11.5% | 0.085-0.115 |
| Tuned Aloe-derived | Aloe Vera Leaf | 0.1-2.00 | 100.00% | 0.1-2.00 |
| Exosomes | Extract | | | |
| BP - Triluronic | Sodium | 0.1-2.00 | 100.00% | 0.1-2.00 |
| Acid Powder | Hyaluronate | | | |
| Allantoin | Allantoin | 0.1-2.00 | 100.00% | 0.1-2.00 |
| Hymagic ™ ® 4D | Water | 0.1-2.00 | up to 100% | 0.1-2.00 |
| | Sodium | | 0.2-0.5% | 0.0004-0.001 |
| | Hyaluronate | | | |
| | Crosspolymer | | | |
| | Sodium | | 0.5-1.0% | 0.001-0.002 |
| | Hyaluronate | | | |
| | Sodium Acetylated | | 0.5-1.2% | 0.001-0.002 |
| | Hyaluronate | | | |
| | Hydrolyzed Sodium | | 0.1-0.2% | 0.0002-0.0004 |
| | Hyaluronate | | | |
| | Pentylene Glycol | | 3-5% | 0.006-0.01 |
| | Ethylhexylglycerin | | 0.05-0.1% | 0.0001-0.00002 |
| Arginine Base | Arginine | 0.01-1.00 | 100.00% | 0.01-1.00 |
| Aloe Vera Gel | Aloe Barbadensis | 0.01-1.00 | 100.00% | 0.01-1.00 |
| 200:1 Conc. | Leaf Extract | | | |
| Ritamectant K2 | Dipotassium | 0.01-1.00 | 100.00% | 0.01-1.00 |
| | Glycyrrhizate | | | |
| Sodium Hydroxide | Sodium Hydroxide | 0.01-1.00 | 100.00% | 0.01-1.00 |
| D-Panthenol | Panthenol | 0.01-1.00 | 100.00% | 0.01-1.00 |
| DermoFeel ® PA-3 | Sodium Phytate | 0.01-1.00 | 100.00% | 0.01-1.00 |
| Ginkgo Biloba | Glycerin | 0.01-1.00 | 40-60% | 0.040-0.060 |
| Extract in Glycerin | Water | | 40-60% | 0.040-0.060 |
| | Ginkgo Biloba Leaf | | 1-10% | 0.001-0.01 |
| | Extract | | | |
| D-3 100 IU/mg | Cholecalciferol | 0.00001-0.001 | 100.00% | 0.00001-0.001 |
| Vitamin E Acetate | Tocopheryl Acetate | 0.00001-0.001 | 100.00% | 0.00001-0.001 |
| Oil | | | | |
| Noni ™ Concentrate | Morinda Citrifolia | 0.0000001-0.0001 | 100.00% | 0.0000001-0.0001 |
| Liquid | Fruit Juice | | | |
| Echinacea Purpurea | Echinacea Purpurea | 0.0000001-0.0001 | 100.00% | 0.0000001-0.0001 |
| | Extract | | | |
| DermalRx ® KBGA | Water | 0.0000001-0.0001 | 44-46% | 0.00000044-0.00000046 |
| | Glycerin | | 44-46% | 0.00000044-0.00000046 |
| | Algae Extract | | 8-12% | 0.000000008-0.00000012 |
| MariMoist ®R10041 | Water | 0.0000001-0.0001 | 89-98% | 0.000000089-0.00000098 |
| | Chondrus Crispus | | 2-10% | 0.000000002-0.00000001 |
| | Extract | | | |
| | Sodium | | 0.01-0.1% | 0.0000000001-0.000000001 |
| | Hyaluronate | | | |
| Agascalm ™ | Propanediol | 0.0000001-0.0001 | 70-90% | 0.00000007-0.00000009 |
| | Glycerin | | 10-30% | 0.00000001-0.00000003 |
| | Agastache | | 0.5-1.5% | 0.000000005-0.000000015 |
| | Mexicana | | | |
| | Flower/Leaf/Stem | | | |
| | Extract | | | |
| Phytessence ™ | Glycerin | 0.0000001-0.0001 | 46-50% | 0.000000046-0.00000005 |
| Wakame | Water | | 46-50% | 0.000000046-0.00000005 |
| | Undaria Pinnatifida | | 5-10% | 0.00000005-0.0000001 |
| | Extract | | | |
| | Phenoxyethanol | | 0.5-2.00% | 0.0000000001-0.00000001 |
| Mitostime ™ | Water | 0.0000001-0.0001 | up to 100% | 0.0000001-0.00001 |
| | Laminaria Digitata | | 5-10% | 0.00000005-0.00000001 |
| | Extract | | | |
| | Phenoxyethanol | | 0.5-2.00% | 0.000000001-0.000001 |
| Ulva Lactuca | Water | 0.0000001-0.0001 | 73-74% | 0.000000073-0.000000074 |
| Organic Biogreen | Glycerin | | 23-24% | 0.000000023-0.000000024 |
| Extract | Ulva Lactuca (Sea | | 1.5-2.5% | 0.0000000015-0.000000025 |
| | Lettuce) Extract | | | |
| | Citric Acid | | 0.1-1% | 0.0000000001-0.00000001 |
| | Sodium Benzoate | | 0.27-0.33% | 0.0000000027-0.0000000033 |
| | Potassium Sorbate | | 0.13-0.17% | 0.0000000013-0.0000000017 |

### Additional Formulation Variants

Additional formulation variants were prepared to address specific intimate care needs. For vaginal dryness and atrophy, the Original EXO Vaginal Gel formulation may be combined with Pueraria mirifica. For vaginal tightening, the Original EXO Vaginal Gel formulation may be combined with one or more of Manjakani (Quercus Infectoria), Sophora Flavescens Ait./Sophorae Flavescentis, Fructus Cnidii extract, Witch Hazel, Radix Stemonae, and Flos Rosae Rugosae. For clitoral stimulation and vaginal sensitization, the Original EXO Vaginal Gel formulation may be combined with one or more of L-arginine HCl, L-citrulline, and Chamomilla recutita.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

### REFERENCES

Basu, J., and Ludlow, J. W. (2016). Exosomes for repair, regeneration and rejuvenation. Expert Opin. Biol. Ther. 16 (4), 489-506. doi:10.1517/14712598.2016. 1131976.
Buehrer, B. M., and Cheatham, B. (2013). Isolation and characterization of human adipose-derived stem cells for use in tissue engineering. Methods Mol. Biol. Clift. N. J. 1001, 1-11. doi:10.1007/978-1-62703-363-3_1
Gruber, H., Somayaji, S., Riley, F., Hoelscher, G. L., Norton, H. J., Ingram, J., et al. (2012). Human adipose-derived mesenchymal stem cells: serial passaging, doubling time and cell senescence. Biotech. Histochem. 87 (4), 303-311. doi:10.3109/10520295. 2011.649785
Gurunathan, S., Kang, M.-H., and Kim, J.-H. (2021). A comprehensive review on factors influences biogenesis, functions, therapeutic and clinical implications of exosomes. Int. J. Nanomedicine 16, 1281-1312. doi:10.2147/IJN.S291956
Hu, L., Wang, J., Zhou, X., Xiong, Z., Zhao, J., Yu, R., et al. (2016). Exosomes derived from human adipose mesenchymal stem cells accelerates cutaneous wound healing via optimizing the characteristics of fibroblasts. Sci. Rep. 6, 32993. doi:10.1038/srep32993
Keating, Armand. Mesenchymal stromal cells. Current Opinion in Hematology 13(6):p 419-425, November 2006.|DOI: 10.1097/01.moh.0000245697.54887.6f
Kim, K. M., Abdelmohsen, K., Mustapic, M., Kapogiannis, D., and Gorospe, M. (2017). RNA in extracellular vesicles. Wiley Interdiscip. Rev. RNA 8 (4), e1413. doi:10. 1002/wrna.1413
Kowal, J., Tkach, M., and Thery, C. (2014). Biogenesis and secretion of exosomes. Curr. Opin. Cell Biol. 29, 116-125. doi:10.1016/j.ceb.2014.05.004
Liu, M., Lei, H., Dong, P., Fu, X., Yang, Z., Yang, Y., et al. (2017). Adipose-derived mesenchymal stem cells from the elderly exhibit decreased migration and differentiation abilities with senescent properties. Cell Transplant. 26 (9), 1505-1519. doi:10.1177/0963689717721221
Lotfy, A., AboQuella, N. M., and Wang, H. (2023). Mesenchymal stromal/stem cell (MSC)-derived exosomes in clinical trials. Stem Cell Res. Ther. 14 (1), 66. doi:10.1186/s13287-023-03287-7
Ludlow, J. W., and Buehrer, B. M. (2022). “Manipulating the cellular environment generates differential extracellular vesicle packaging and biological activity,” in Advances in exosome research. Available online at: https://www.biocompare.com/3262-eBooks/583797-Advances-in-Exosome-Research/?arev=true (Accessed May 25, 2025).
Ludlow, J. W., and Buehrer, B. M. (2024). Analyses and utilization of selectively tuned human adipose-derived stromal/stem cell exosomes. Methods Mol. Biol. Clift. N. J. 2783, 309-322. doi:10.1007/978-1-0716-3762-3_22
National Library of Medicine (2025). ClinicalTrials.gov: Search results for “exosome therapy”. Available online at: clinicaltrials.gov/search?intr-exosome % 20 therapy.
Pirmoradi, S., Fathi, E., Farahzadi, R., Pilehvar-Soltanahmadi, Y., and Zarghami, N. (2018). Curcumin affects adipose tissue-derived mesenchymal stem cell aging through TERT gene expression. Drug Res. 68 (4), 213-221. doi:10.1055/s-0043-119635
Pote, M. S., and Gacche, R. N. (2025). Exosomal signaling in cancer metastasis: molecular insights and therapeutic opportunities. Archives Biochem. Biophysics 764, 110277. doi:10.1016/j.abb.2024.110277
Qiu, H., Liu, S., Wu, K., Zhao, R., Cao, L., and Wang, H. (2020). Prospective application of exosomes derived from adipose-derived stem cells in skin wound healing: a review. J. Cosmet. Dermatology 19 (3), 574-581. doi:10.1111/jocd.13215
Rani, S., and Ritter, T. (2016). The exosome—a naturally secreted nanoparticle and its application to wound healing. Adv. Mater. Deerf Beach, Fla. 28 (27), 5542-5552. doi:10.1002/adma.201504009
Shabbir, A., Cox, A., Rodriguez-Menocal, L., Salgado, M., and Van Badiavas, E. (2015). Mesenchymal stem cell exosomes induce proliferation and migration of normal and chronic wound fibroblasts, and enhance angiogenesis in vitro. Stem Cells Dev. 24 (14), 1635-1647. doi:10.1089/scd.2014.0316
Shi, H., Wang, M., Sun, Y., Yang, D., Xu, W., and Qian, H. (2021). Exosomes: emerging cell-free based therapeutics in dermatologic diseases. Front. Cell Dev. Biol. 9, 736022. doi:10.3389/fcell.2021.736022
Siraj, Y., Galderisi, U., and Alessio, N. (2023). Senescence induces fundamental changes in the secretome of mesenchymal stromal cells (MSCs): implications for the therapeutic use of MSCs and their derivates. Front. Bioeng. Biotechnol. 11, 1148761. doi:10.3389/fbioe.2023.1148761
Skotland, T., Hessvik, N. P., Sandvig, K., and Llorente, A. (2019). Exosomal lipid composition and the role of ether lipids and phosphoinositides in exosome biology. J. Lipid Res. 60 (1), 9-18. doi:10.1194/jlr.R084343
Smirnova, A., Yatsenko, E., Baranovskii, D., and Klabukov, I. (2023). Mesenchymal stem cell-derived extracellular vesicles in skin wound healing: the risk of senescent drift induction in secretome-based therapeutics. Mil. Med. Res. 10 (1), 60. doi:10.1186/s40779-023-00498-0
Vymetalova, L., Kucirkova, T., Knopfova, L., Pospisilova, V., Kasko, T., Lejdarova, H., et al. (2020). Large-scale automated hollow-fiber bioreactor expansion of umbilical cord-derived human mesenchymal stromal cells for neurological disorders. Neurochem. Res. 45 (1), 204-214. doi:10.1007/si 1064-019-02925-y
Wagner, W., Wein, F., Seckinger, A., Frankhauser, M., Wirkner, U., Krause, U., et al. (2005). Comparative characteristics of mesenchymal stem cells from human bone marrow, adipose tissue, and umbilical cord blood. Exp. Hematol. 33 (11), 1402-1416. doi:10.1016/j.exphem.2005.07.003
Wagner, W., Horn, P., Castoldi, M., Diehlmann, A., Bork, S., Saffrich, R., et al. (2008). Replicative senescence of mesenchymal stem cells: a continuous and organized process. PloS One 3 (5), e2213. doi:10.1371/journal.pone.0002213
Welsh, J. A., Goberdhan, D. C. I., O'Driscoll, L., Buzas, E. I., Blenkiron, C., Bussolati, B., et al. (2024). Minimal information for studies of extracellular vesicles (MISEV2023): from basic to advanced approaches. J. Extracell. Vesicles 13 (2), e12404. doi:10.1002/jev2.12404
Zhang, J., Guan, J., Niu, X., Hu, G., Guo, S., Li, Q., et al. (2015). Exosomes released from human induced pluripotent stem cells-derived MSCs facilitate cutaneous wound healing by promoting collagen synthesis and angiogenesis. J. Transl. Med. 13, 49. doi:10.1186/s12967-015-0417-0
Zhang, Y., Liu, Y., Liu, H., and Tang, W. H. (2019). Exosomes: biogenesis, biologic function and clinical potential. Cell Biosci. 9, 19. doi:10.1186/s13578-019-0282-2
Zhang, Y., Bi, J., Huang, J., Tang, Y., and Du, S. (2020). Exosome: a review of its classification, isolation techniques, storage, diagnostic and targeted therapy applications. Int. J. Nanomedicine 15, 6917-6934. doi:10.2147/IJN.S264498
Zhang, Y., Lan, M., and Chen, Y. (2024). Minimal information for studies of extracellular vesicles (MISEV): ten-year evolution (2014-2023). Pharmaceutics 16 (11), 1394. doi:10.3390/pharmaceutics16111394
Zou, Z., Li, H., Xu, G., Hu, Y., Zhang, W., and Tian, K. (2023). Current knowledge and future perspectives of exosomes as nanocarriers in diagnosis and treatment of diseases. Int. J. Nanomedicine 18, 4751-4778. doi:10.2147/IJN.S417422

What is claimed is:

1. A composition comprising a purified population of extracellular vesicles (EVs) isolated from one or more biological samples,
wherein the size of the EVs is about 50 nm to about 200 nm, inclusive,
wherein the purified population of EVs comprise a tuned cargo comprising:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p; and
(ii) a signature of one or more proteins selected from the group consisting of BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, WNT5A,
wherein the tuned cargo of the EVs is produced by exposure of the one or more biological samples to one or more stress conditions that cause the biological sample to modulate the EV cargo composition.

2. The composition of claim 1, wherein the one or more biological samples comprise a biological sample derived from a tissue, a biological sample derived from cells maintained in culture, a biological sample derived from plants, or a combination thereof.

3. The composition of claim 2, wherein the biological sample derived from the tissue comprises tissue explants maintained ex vivo.

4. The composition of claim 2, wherein the one or more biological samples comprise a biological sample derived from adipose-derived stem cells, a biological sample derived from Aloe vera plants, or a combination thereof.

5. The composition of claim 4, wherein the adipose-derived stem cells are cultured in a bioreactor.

6. The composition of claim 4, wherein the adipose-derived stem cells retain mesenchymal stromal cell (MSC) characteristics comprising:
(i) adherence to plastic in standard culture conditions;
(ii) a surface marker phenotype comprising CD105+, CD73+, CD90+, CD45−, CD34−, CD14− or CD11b−, CD79α− or CD19−, and HLA-DR−; and
(iii) a capacity to differentiate into bone, adipose, and cartilage cells.

7. The composition of claim 1, wherein the one or more stress conditions comprise hypoxic stress, metabolic stress, mechanical stress, osmotic stress, chemical stress, heat stress, or a combination thereof.

8. The composition of claim 7, wherein the tuned cargo of the purified population of extracellular vesicles is produced by exposure of the one or more biological samples to heat stress, wherein the heat stress comprises exposing the one or more biological samples to temperatures between about 30 °C and about 50 °C, inclusive for a period of time between about 10 minutes and about 6 hours, inclusive.

9. The composition of claim 8, wherein the heat stress further comprises subsequently incubating the one or more biological samples at a temperature of about 36 °C-38 °C, inclusive, for about 24 hours-72 hours, inclusive.

10. The composition of claim 8, wherein the heat stress further comprises subsequently incubating the one or more biological samples at a temperature of about 18 °C-25 °C, inclusive, for about 1 hours-36 hours, inclusive.

11. The composition of claim 1, wherein the tuned cargo of the purified population of extracellular vesicles modulates one or more biological activities of a recipient cell, wherein the recipient cell is a human cell.

12. The composition of claim 11, wherein the biological activities comprise one or more of:
(i) modulation of gene expression;
(ii) modulation of collagen production;
(iii) modulation of cytokine production;
(iv) modulation of cell proliferation;
(v) modulation of extracellular matrix remodeling; or
(vi) modulation of oxidative stress.

13. The composition of claim 1, wherein the composition:
(i) is a nutraceutical composition comprising a dietary amount of the purified population of extracellular vesicles comprising the tuned cargo;
(ii) is a cosmetic composition comprising a cosmetic amount of the purified population of extracellular vesicles comprising the tuned cargo and a cosmetically acceptable carrier; or
(iii) is a cosmeceutical composition comprising a cosmeceutical amount of the purified population of extracellular vesicles comprising the tuned cargo and a cosmeceutically acceptable carrier; or
(iv) is a pharmaceutical composition comprising a therapeutic amount of the purified population of extracellular vesicles comprising the tuned cargo and a pharmaceutically acceptable carrier.

14. A method for improving appearance of human skin comprising:
(i) obtaining a biological sample comprising a population of extracellular vesicles (EVs) comprising a cargo composition;
(ii) modulating the cargo composition by exposing the biological sample comprising the population of EVs comprising the cargo composition to one or more stress conditions to form a population of EVs comprising a tuned cargo composition;
(iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive;
(iv) preparing a cosmetic composition containing a cosmetically acceptable carrier and about 1×10E8 to about 1×10E12, inclusive, of the EVs comprising the tuned cargo, and
(v) applying the composition topically to human skin,
wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof; and
(ii) a signature of one or more proteins selected from the group consisting BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, WNT5A.

15. The method of claim 14, wherein the one or more biological samples comprise a biological sample derived from a tissue, a biological sample derived from cells maintained in culture, a biological sample derived from plants, or a combination thereof.

16. The method of claim 15, wherein the biological sample derived from a tissue comprises tissue explants maintained ex vivo.

17. The method of claim 16, wherein the one or more biological samples comprise a biological sample derived from adipose-derived stem cells, a biological sample derived from Aloe vera plants, or a combination thereof.

18. The method of claim 17, wherein the adipose-derived stem cells are cultured in a bioreactor.

19. The method of claim 17, wherein the adipose-derived stem cells retain mesenchymal stromal cell (MSC) characteristics comprising:
(i) adherence to plastic in standard culture conditions;
(ii) a surface marker phenotype comprising CD105+, CD73+, CD90+, CD45−, CD34−, CD14− or CD11b−, CD79α− or CD19−, and HLA-DR−; and
(iii) a capacity to differentiate into bone, adipose, and cartilage cells.

20. The method of claim 14, wherein the one or more stress conditions comprise hypoxic stress, metabolic stress, mechanical stress, osmotic stress, chemical stress, heat stress, or a combination thereof.

21. The method of claim 20, wherein the tuned cargo of the purified population of extracellular vesicles is produced by exposure of the one or more biological samples to heat stress, wherein the heat stress comprises exposing the one or more biological samples to temperatures between about 30 °C to about 50 °C, inclusive for a period of time between 10 minutes to about 6 hours, inclusive.

22. The composition of claim 21, wherein the heat stress further comprises subsequently incubating the one or more biological samples at a temperature of about 36 °C-38 °C, inclusive, for about 24 hours-72 hours, inclusive.

23. The composition of claim 21, wherein the heat stress further comprises subsequently incubating the one or more biological samples at a temperature of about 18 °C-25 °C, inclusive, for about 1 hours-36 hours, inclusive.

24. The method of claim 14, wherein administering the composition comprising purified population of extracellular vesicles comprising the tuned cargo improves one or more parameters of skin appearance.

25. The method of claim 24, wherein the one or more parameters of skin appearance comprise:
(i) reducing the appearance of wrinkles;
(ii) reducing skin roughness;
(iii) reducing wrinkle depth volume;
(iv) reducing wrinkle depth value;
(v) increasing skin firmness;
(vi) increasing skin luminosity; or
(vii) improving one or more symptoms associated with acne.

26. A method for treating a skin condition, wherein the skin condition is eczema comprising:
(i) obtaining a biological sample comprising a population of extracellular vesicles (EVs) comprising a cargo composition;
(ii) modulating the cargo composition by exposing the biological sample comprising the population of EVs comprising the cargo composition to one or more stress conditions to form a population of EVs comprising a tuned cargo composition;
(iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive;
(iv) preparing a cosmetic composition containing a cosmetically acceptable carrier and about 1×10E8 to about 1×10E12, inclusive, of the EVs comprising the tuned cargo, and
(v) applying the composition topically to a region of the skin affected by eczema, wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof; and
(ii) a signature of one or more proteins selected from the group consisting BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, or WNT5A.

27. The method of claim 26, wherein the one or more biological samples comprise a biological sample derived from a tissue, a biological sample derived from cells maintained in culture, a biological sample derived from plants, or a combination thereof.

28. The method of claim 27, wherein the biological sample derived from a tissue comprises tissue explants maintained ex vivo.

29. The method of claim 28, wherein the one or more biological samples comprise a biological sample derived from adipose-derived stem cells, a biological sample derived from Aloe vera plants, or a combination thereof.

30. The method of claim 29, wherein the adipose-derived stem cells are cultured in a bioreactor.

31. The method of claim 29, wherein the adipose-derived stem cells retain mesenchymal stromal cell (MSC) characteristics comprising:
(i) adherence to plastic in standard culture conditions;
(ii) a surface marker phenotype comprising CD105+, CD73+, CD90+, CD45−, CD34−, CD14− or CD11b−, CD79α− or CD19−, and HLA-DR−; and
(iii) a capacity to differentiate into bone, adipose, and cartilage cells.

32. The method of claim 26, wherein the one or more stress conditions comprise hypoxic stress, metabolic stress, mechanical stress, osmotic stress, chemical stress, heat stress, or a combination thereof.

33. The method of claim 32, wherein the tuned cargo of the purified population of extracellular vesicles is produced by exposure of the one or more biological samples to heat stress, wherein the heat stress comprises exposing the one or more biological samples to temperatures between about 30 °C to about 50 °C, inclusive for a period of time between 10 minutes to about 6 hours, inclusive.

34. The composition of claim 29, wherein the heat stress further comprises subsequently incubating the one or more biological samples at a temperature of about 36 °C-38 °C, inclusive, for about 24 hours-72 hours, inclusive.

35. The composition of claim 29, wherein the heat stress further comprises subsequently incubating the one or more biological samples at a temperature of about 18 °C-25 °C, inclusive, for about 1 hours-36 hours, inclusive.

36. The method of claim 26, wherein administering the composition comprising purified population of extracellular vesicles comprising the tuned cargo improves one or more symptoms associated with eczema.

37. The method of claim 36, wherein the one or more symptoms associated with eczema comprise:
(i) inflammation;
(ii) redness;
(iii) scaling;
(iv) dryness;
(v) irritation; or
(vi) itchiness.

38. A method for promoting wound healing or skin repair comprising:
(i) obtaining a biological sample comprising a population of extracellular vesicles (EVs) comprising a cargo composition;
(ii) modulating the cargo composition by exposing the biological sample comprising the population of EVs comprising the cargo composition to one or more stress conditions to form a population of EVs comprising a tuned cargo composition;
(iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive;
(iv) preparing a cosmetic composition containing a cosmetically acceptable carrier and about 1×10E8 to about 1×10E12, inclusive, of the EVs comprising the tuned cargo, and
(v) applying the composition topically to a wound,
wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof, and
(ii) a signature of one or more proteins selected from the group consisting BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, WNT5A.

39. The method of claim 38, wherein the one or more biological samples comprise a biological sample derived from a tissue, a biological sample derived from cells maintained in culture, a biological sample derived from plants, or a combination thereof.

40. The method of claim 39, wherein the biological sample derived from a tissue comprises tissue explants maintained ex vivo.

41. The method of claim 40, wherein the one or more biological samples comprise a biological sample derived from adipose-derived stem cells, a biological sample derived from Aloe vera plants, or a combination thereof.

42. The method of claim 41, wherein the adipose-derived stem cells are cultured in a bioreactor.

43. The method of claim 41, wherein the adipose-derived stem cells retain mesenchymal stromal cell (MSC) characteristics comprising:
(i) adherence to plastic in standard culture conditions;
(ii) a surface marker phenotype comprising CD105+, CD73+, CD90+, CD45−, CD34−, CD14− or CD11b−, CD79α− or CD19−, and HLA-DR−; and
(iii) a capacity to differentiate into bone, adipose, and cartilage cells.

44. The method of claim 38, wherein the one or more stress conditions comprise hypoxic stress, metabolic stress, mechanical stress, osmotic stress, chemical stress, heat stress, or a combination thereof.

45. The method of claim 38, wherein the tuned cargo of the purified population of extracellular vesicles is produced by exposure of the one or more biological samples to heat stress, wherein the heat stress comprises exposing the one or more biological samples to temperatures between about 30 °C to about 50 °C, inclusive for a period of time between 10 minutes to about 6 hours, inclusive.

46. The composition of claim 45, wherein the heat stress further comprises subsequently incubating the one or more biological samples at a temperature of about 36 °C-38 °C, inclusive, for about 24 hours-72 hours, inclusive.

47. The composition of claim 45, wherein the heat stress further comprises subsequently incubating the one or more biological samples at a temperature of about 18 °C-25 °C, inclusive, for about 1 hours-36 hours, inclusive.

48. The method of claim 38, wherein administering the composition comprising purified population of extracellular vesicles comprising the tuned cargo modulates one or more parameters associated with wound healing or skin repair.

49. The method of claim 48, wherein the one or more parameters associated with wound healing comprise:
(i) increasing collagen production;
(ii) increasing cell proliferation;
(iii) promoting angiogenesis;
(iv) promoting immune regulation and reducing inflammation; or
(v) promoting tissue regeneration and wound closure.

50. The method of claim 38, wherein administering the composition comprising purified population of extracellular vesicles comprising the tuned cargo improves one or more symptoms associated with gum recession.

51. A method for promoting hair health comprising:
(i) obtaining a biological sample comprising a population of extracellular vesicles (EVs) comprising a cargo composition;
(ii) modulating the cargo composition by exposing the biological sample comprising the population of EVs comprising the cargo composition to one or more stress conditions to form a population of EVs comprising a tuned cargo composition;
(iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive;
(iv) preparing a cosmetic composition containing a cosmetically acceptable carrier and about 1×10E8 to about 1×10E12, inclusive, of the EVs comprising the tuned cargo; and
(v) applying the composition topically to hair, scalp, eyebrows, eyelashes, or a combination thereof,
wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof, and
(ii) a signature of one or more proteins selected from the group consisting BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, or WNT5A.

52. The method of claim 51, wherein the one or more biological samples comprise a biological sample derived from a tissue, a biological sample derived from cells maintained in culture, a biological sample derived from plants, or a combination thereof.

53. The method of claim 52, wherein the biological sample derived from a tissue comprises tissue explants maintained ex vivo.

54. The method of claim 53, wherein the one or more biological samples comprise a biological sample derived from adipose-derived stem cells, a biological sample derived from Aloe vera plants, or a combination thereof.

55. The method of claim 54, wherein the adipose-derived stem cells are cultured in a bioreactor.

56. The method of claim 55, wherein the adipose-derived stem cells retain mesenchymal stromal cell (MSC) characteristics comprising:
(i) adherence to plastic in standard culture conditions;
(ii) a surface marker phenotype comprising CD105+, CD73+, CD90+, CD45−, CD34−, CD14− or CD11b−, CD79α− or CD19−, and HLA-DR−; and
(iii) capacity to differentiate into bone, adipose, and cartilage cells.

57. The method of claim 51, wherein the one or more stress conditions comprise hypoxic stress, metabolic stress, mechanical stress, osmotic stress, chemical stress, heat stress, or a combination thereof.

58. The method of claim 57, wherein the tuned cargo of the purified population of extracellular vesicles is produced by exposure of the one or more biological samples to heat stress, wherein the heat stress comprises exposing the one or more biological samples to temperatures between about 30 °C to about 50 °C, inclusive for a period of time between 10 minutes to about 6 hours, inclusive.

59. The composition of claim 58, wherein the heat stress further comprises subsequently incubating the one or more biological samples at a temperature of about 36 °C-38 °C, inclusive, for about 24 hours-72 hours, inclusive.

60. The composition of claim 58, wherein the heat stress further comprises subsequently incubating the one or more biological samples at a temperature of about 18 °C-25 °C, inclusive, for about 1 hours-36 hours, inclusive.

61. The method of claim 51, wherein administering the composition comprising purified population of extracellular vesicles comprising the tuned cargo modulates one or more parameters associated with hair health.

62. The method of claim 61, wherein the one or more parameters associated with hair health comprise:
(i) decreasing hair loss;
(ii) increasing hair density;
(iii) increasing appearance of hair thickness;
(iv) improving scalp health;
(v) improving hair shine; or
(vi) improving hair volume.

63. A method for promoting vaginal health comprising:
(i) obtaining a biological sample comprising a population of extracellular vesicles (EVs) comprising a cargo composition;
(ii) modulating the cargo composition by exposing the biological sample comprising the population of EVs comprising the cargo composition to one or more stress conditions to form a population of EVs comprising a tuned cargo composition;
(iii) purifying, from the biological sample exposed to one or more stress conditions, the population of extracellular vesicles (EVs) comprising the tuned cargo composition, wherein the population of EVs comprises EVs of diameter about 50 nm-500 nm, inclusive;
(iv) preparing a cosmetic composition containing a cosmetically acceptable carrier and about 1×10E8 to about 1×10E12, inclusive, of the EVs comprising the tuned cargo, and
(v) applying the composition topically to vaginal skin,
wherein the tuned cargo of the purified population of EVs comprises:
(i) a signature of one or more miRNAs selected from hsa-miR-100-5p, hsa-miR-10b-5p, hsa-miR-143-3p, hsa-miR-10a-5p, hsa-miR-22-3p, hsa-miR-125b-5p, hsa-miR-21-5p, hsa-miR-99b-5p, hsa-miR-125a-5p, hsa-let-7i-5p, hsa-miR-146b-5p, hsa-miR-99a-5p, hsa-let-7f-5p, hsa-miR-24-3p, hsa-let-7a-5p, hsa-miR-26a-5p, hsa-miR-148a-3p, hsa-miR-1246, hsa-miR-199b-3p, hsa-miR-199a-3p, hsa-miR-199a-5p, hsa-miR-23a-3p, hsa-miR-92a-3p, hsa-miR-127-3p, hsa-miR-152-3p, hsa-miR-146a-5p, hsa-let-7g-5p, hsa-miR-191-5p, hsa-miR-29a-3p, hsa-miR-151a-3p, hsa-let-7b-5p, hsa-miR-199b-5p, hsa-miR-145-3p, hsa-miR-221-3p, hsa-miR-222-3p, hsa-miR-27b-3p, hsa-miR-30d-5p, hsa-miR-25-3p, hsa-miR-101-3p, hsa-miR-221-5p, hsa-miR-423-3p, hsa-miR-186-5p, hsa-miR-30a-5p, hsa-miR-151a-5p, hsa-miR-151b, hsa-miR-27a-3p, hsa-miR-224-5p, hsa-miR-181a-5p, hsa-miR-28-3p, hsa-miR-320a, hsa-miR-378a-3p, hsa-miR-155-5p, hsa-miR-193a-5p, hsa-let-7c-5p, hsa-miR-21-3p, hsa-miR-196a-5p, hsa-miR-148b-3p, hsa-miR-409-5p, hsa-miR-92b-3p, hsa-miR-140-3p, hsa-miR-486-5p, hsa-miR-30e-5p, hsa-miR-615-3p, hsa-miR-409-3p, hsa-miR-501-3p, hsa-miR-192-5p, hsa-miR-103a-3p, hsa-miR-26b-5p, hsa-miR-1271-5p, hsa-miR-500a-3p, hsa-miR-145-5p, hsa-miR-484, hsa-miR-125b-2-3p, hsa-miR-361-5p, hsa-miR-503-5p, and hsa-miR-379-5p, or a combination thereof; and
(ii) a signature of one or more proteins selected from the group consisting BMP2, SOX2, IGF1, CDKN2A, TCF4, AR, LEP, TGFB2, KITLG, NOG, BMP4, DCN, STAT5A, STAT6, SFRP1, AXIN2, HEY1, IL1B, MK167, HSP27, HSP60, HSP70, HSP90, CD9, CD63, CD81, CD82, or WNT5A.

64. The method of claim 63, wherein the one or more biological samples comprise a biological sample derived from a tissue, a biological sample derived from cells maintained in culture, a biological sample derived from plants, or a combination thereof.

65. The method of claim 64, wherein the biological sample derived from a tissue comprises tissue explants maintained ex vivo.

66. The method of claim 65, wherein the one or more biological samples comprise a biological sample derived from adipose-derived stem cells, a biological sample derived from Aloe vera plants, or a combination thereof.

67. The method of claim 66, wherein the adipose-derived stem cells are cultured in a bioreactor.

68. The method of claim 66, wherein the adipose-derived stem cells retain mesenchymal stromal cell (MSC) characteristics comprising:
(i) adherence to plastic in standard culture conditions;
(ii) a surface marker phenotype comprising CD105+, CD73+, CD90+, CD45−, CD34−, CD14− or CD11b−, CD79α− or CD19−, and HLA-DR−; and
(iii) a capacity to differentiate into bone, adipose, and cartilage cells.

69. The method of claim 63, wherein the one or more stress conditions comprise hypoxic stress, metabolic stress, mechanical stress, osmotic stress, chemical stress, heat stress, or a combination thereof.

70. The method of claim 69, wherein the tuned cargo of the purified population of extracellular vesicles is produced by exposure of the one or more biological samples to heat stress, wherein the heat stress comprises exposing the one or more biological samples to temperatures between about 30 °C to about 50 °C, inclusive for a period of time between 10 minutes to about 6 hours, inclusive.

71. The composition of claim 70, wherein the heat stress further comprises subsequently incubating the one or more biological samples at a temperature of about 36 °C-38 °C, inclusive, for about 24 hours-72 hours, inclusive.

72. The composition of claim 70, wherein the heat stress further comprises subsequently incubating the one or more biological samples at a temperature of about 18 °C-25 °C, inclusive, for about 1 hours-36 hours, inclusive.

73. The method of claim 63, wherein administering the composition comprising purified population of extracellular vesicles comprising the tuned cargo modulates one or more parameters associated with vaginal health.

74. The method of claim 73, wherein the one or more parameters associated with vaginal health comprise:
(i) tissue regeneration and renewal;
(ii) increasing collagen production;
(iii) extracellular matrix remodeling;
(iv) promoting angiogenesis;
(v) reducing inflammation;
(vi) reducing vaginal dryness; or
(vii) increasing vaginal sensitization.
